(19) United States
(12) Patent Application Publication
(10) Pub. No.: US 2026/0035361 A1
(43) Pub. Date: Feb. 5, 2026

(54) COMPOUNDS FOR MODULATING HUR (ELAVL1)

(71) Applicant: SHANGHAI DEGRON BIOMEDICAL TECHNOLOGY CO. LTD., Shanghai (CN)

(72) Inventors: Lihui ZOU, Shanghai (CN); Zheng YANG, Shanghai (CN); Zhuming SUN, Shanghai (CN); Hexiu SU, Shanghai (CN); Chenlu GENG, Shanghai (CN); Hao DOU, Shanghai (CN)

(21) Appl. No.: 19/133,275

(22) PCT Filed: Nov. 28, 2023
(86) PCT No.: PCT/CN2023/134754

§ 371 Date: May 28, 2025

(30) Foreign Application Priority Data
Nov. 28, 2022 (WO) PCT/CN2022/164645

(51) Int. Cl. C07D 405/14; A61K 31/4525; A61K 31/454; A61K 31/4545; A61K 31/4709; A61K 31/496; A61K 31/497; A61K 31/501; A61K 31/506; A61K 31/517; A61K 31/519; A61K 31/522; A61K 31/5377; A61K 31/675; C07B 59/00; C07D 409/14; C07D 413/04; C07D 413/14; C07D 417/14; C07D 471/04; C07D 473/08; C07D 491/048; C07D 491/107; C07D 513/04; C07F 9/6558

(52) U.S. Cl. CPC C07D 405/14; A61K 31/4525; A61K 31/454; A61K 31/4545; A61K 31/4709; A61K 31/496; A61K 31/497; A61K 31/501; A61K 31/506; A61K 31/517; A61K 31/519; A61K 31/522; A61K 31/5377; A61K 31/675; C07B 59/002; C07D 409/14; C07D 413/04; C07D 413/14; C07D 417/14; C07D 471/04; C07D 473/08; C07D 491/048; C07D 491/107; C07D 513/04; C07F 9/65586

(57) ABSTRACT

The application relates to a compound of Formula I:

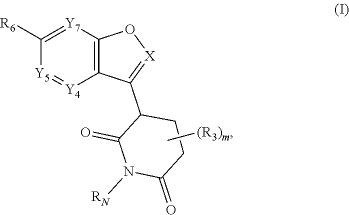

or a pharmaceutically acceptable salt, hydrate, solvate, prodrug, stereoisomer, or tautomer thereof, which degrades HuR, a pharmaceutical composition comprising a compound of Formula I, and a method of treating or preventing a disease in which HuR plays a role.

### RELATED APPLICATION

This application claims priority to, and the benefit of, PCT Application No. PCT/CN_{2022/134645}, filed on Nov. 28, 2022, the entire contents of which are incorporated herein by reference.

### BACKGROUND

Post-transcriptional gene regulation occurs at the levels of pre-mRNA splicing and maturation, as well as mRNA transport, editing, storage, stability, and translation. This level of gene regulation is essential for normal development, but when dysregulated, has many implications in disease conditions, including cancer. These functions are mediated by RNA-binding proteins (RBPs).

The RBP Hu antigen R (“HuR”) is a member of the embryonic lethal abnormal vision (“ELAV”) family that binds to adenine- and uridine-rich elements (collectively, “ARE”) located in the 3′- or 5′-untranslated region (“UTR”) of target mRNAs. HuR is elevated in a broad range of cancer tissues compared with the corresponding normal tissues. In early reports, upregulated HuR in brain and colon cancers was linked to the enhanced expression of COX-2, VEGF, TGF-β, IL-8, and other cancer-associated proteins. Subsequent studies revealed that HuR was broadly overexpressed in virtually all malignancies tested, including cancers of the colon, prostate, breast, brain, ovaries, pancreas, and lung. Elevated cytoplasmic accumulation of HuR correlates with high-grade malignancy and serves as a prognostic factor of poor clinical outcome in those cancers. Moreover, HuR is proposed to play a causal role in tumor development/progression. Cancer cells with elevated HuR produced significantly larger tumors than those arising from control populations in a mouse xenograft model, while reduced HuR level led to decreased tumor size.

Although there are many examples of compounds which specifically interfere with protein-protein interactions, there is limited success of drug discovery for protein-RNA interactions, especially for HuR. The present application addresses the need.

### SUMMARY

The present application provides novel HuR degraders that modulate the activities of HuR through its degradation, which are useful in the treatment of a disease or disorder in which HuR plays a role.

A first aspect of the application relates to a compound of Formula I:

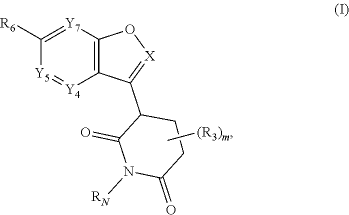

or a pharmaceutically acceptable salt, solvate, prodrug, stereoisomer, or tautomer thereof, wherein the variables in Formula I are described herein.

Another aspect of the application relates to a pharmaceutical composition comprising a compound described herein, or a pharmaceutically acceptable salt, solvate, prodrug, stereoisomer, or tautomer thereof, and a pharmaceutically acceptable diluent, excipient, or carrier.

Another aspect of the application relates to a method of treating or preventing a disease or disorder (e.g., a HuR-mediated disease or disorder or a disease or disorder in which HuR plays a role), as described herein (e.g., bladder cancer, brain cancer, breast cancer, colorectal cancer, cervical cancer, gastrointestinal cancer, genitourinary cancer, glioma, head and neck cancer, lung cancer, ovarian cancer, pancreatic cancer, prostate cancer, renal cancer, skin cancer, or testicular cancer). The method comprises administering to a subject in need thereof a therapeutically effective amount of a compound described herein, or a pharmaceutically acceptable salt, solvate, prodrug, stereoisomer, or tautomer thereof, or a therapeutically effective amount of a pharmaceutical composition described herein.

Another aspect of the application relates to a compound described herein, or a pharmaceutically acceptable salt, solvate, prodrug, stereoisomer, or tautomer thereof, or a pharmaceutical composition described herein, for use in a method of treating or preventing a disease or disorder or of modulating (e.g., deactivating or degrading) HuR.

Another aspect of the application relates to use of a compound described herein, or a pharmaceutically acceptable salt, solvate, prodrug, stereoisomer, or tautomer thereof, or a pharmaceutical composition described herein, in the manufacture of a medicament for treating or preventing a disease or disorder or for modulating (e.g., deactivating or degrading) HuR.

The present application provides degraders of HuR that are therapeutic agents in the treatment of diseases or disorders associated with the modulation of HuR.

The present application further provides compounds and compositions with an improved therapeutic profile (e.g., efficacy, pharmacodynamics, safety) relative to known HuR degraders and alternative routes of administration, toward the treatment of diseases associated with HuR.

### DETAILED DESCRIPTION

### Compounds of the Application

The present application relates to compounds and compositions thereof that are capable of degrading HuR. The application features methods of treating, preventing, or ameliorating a disease or disorder in which HuR plays a role by administering to a subject in need thereof a therapeutically effective amount of a compound or a composition described herein. The compounds or compositions of the present application can be used in the treatment of a variety of HuR-mediated diseases and disorders by degrading HuR.

In one aspect, the present application relates to a compound of Formula I:

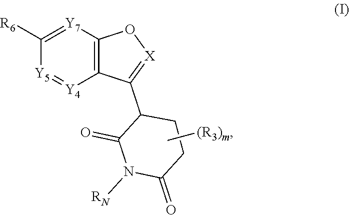

or a pharmaceutically acceptable salt, solvate, prodrug, stereoisomer, or tautomer thereof, wherein:
X is N or CR_{X};
Y₄ is N or CR₄;
Y₅ is N or CR₅;
Y₇ is N or CR₇, provided that no more than one of Y₄, Y₅, and Y₇ is N;
R_{X}, R₄, R₅, and R₇ each independently is H, C₁-C₆ alkyl, C₁-C₆ haloalkyl, or halogen;
m is 0, 1, 2, 3, or 4;
each R₃ is independently C₁-C₆ alkyl, C₁-C₆ haloalkyl, C₁-C₆ alkoxy, C₁-C₆ haloalkoxy, OH, or halogen;
R_{N} is H or C₁-C₆ alkyl;
R₆ is R₆′ or (CH₂)₁₋₃—R₆′;
R₆′ is heterocyclyl comprising a 4- to 6-membered ring comprising a nitrogen atom and optionally additional 1 to 3 heteroatoms selected from N, O, and S, C₆-C₁₀ aryl, heteroaryl comprising a 5- or 6-membered ring comprising a nitrogen atom and optionally additional 1 to 3 heteroatoms selected from N, O, and S, or fused-ring heteroaryl comprising a 5- or 6-membered ring comprising a nitrogen atom and optionally additional 1 to 3 heteroatoms selected from N, O, and S and a 5- or 6-membered ring optionally comprising 1 to 4 heteroatoms selected from N, O, and S, wherein the heterocyclyl, aryl, heteroaryl, or fused-ring heteroaryl is optionally substituted with one or more R₆₁; and
each R₆₁ is independently
   C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
   C₁-C₆ alkoxy, C₁-C₆ haloalkoxy, C₁-C₆ alkyl amino, di-C₁-C₆ alkyl amino, OH, amino, NO₂, halogen, CN, ═O,
   C(O)R₆₂, C(O)OR₆₂, C(O)NR₆₃R₆₄,
   L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
      C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms, wherein:
   L is absent, or a linker selected from O, (CR_{L1}R_{L2})ₙ, O—(CR_{L1}R_{L2})ₙ, (CR_{L1}R_{L2})ₙ—O, and S(O)₂;
   each R_{L1} and each R_{L2} is independently H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
   each R_{A} is independently halogen, OH, C₁-C₆ alkyl, C₁-C₆ alkoxy, C₁-C₆ alkyl amino, or di-C₁-C₆ alkyl amino; and
   n is 1, 2, or 3; or
two R₆₁, together with the carbon atom(s) to which they are attached, may form C₄-C₁₀ carbocyclyl, heterocyclyl comprising one 4- to 6-membered and 1 to 3 heteroatoms selected from N, O, and S, spiro- or fused-heterocyclyl comprising two 4- to 6-membered rings comprising 1 to 4 heteroatoms selected from N, O, and S, or C₆ aryl, wherein the carbocyclyl, heterocyclyl, spiro- or fused-heterocyclyl, or aryl is optionally substituted with one or more groups independently selected from C₁-C₆ alkyl, C₁-C₆ haloalkyl, C₁-C₆ alkoxy, C₁-C₆ haloalkoxy, C₁-C₆ alkyl amino, di-C₁-C₆ alkyl amino, OH, amino, halogen, CN, and —O; and each R₆₂, each R₆₃, and each R₆₄ is independently H, C₁-C₆ alkyl, or C₁-C₆ haloalkyl.

In some embodiments, a compound of Formula I is of any one of Formulae II-IX:

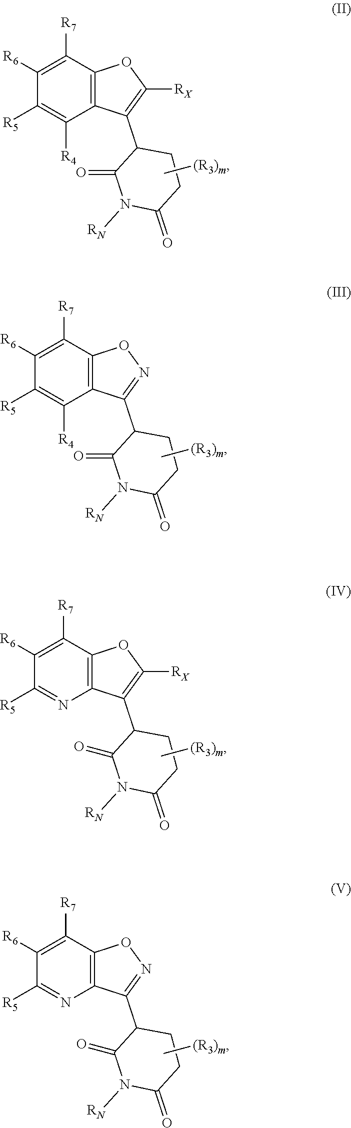

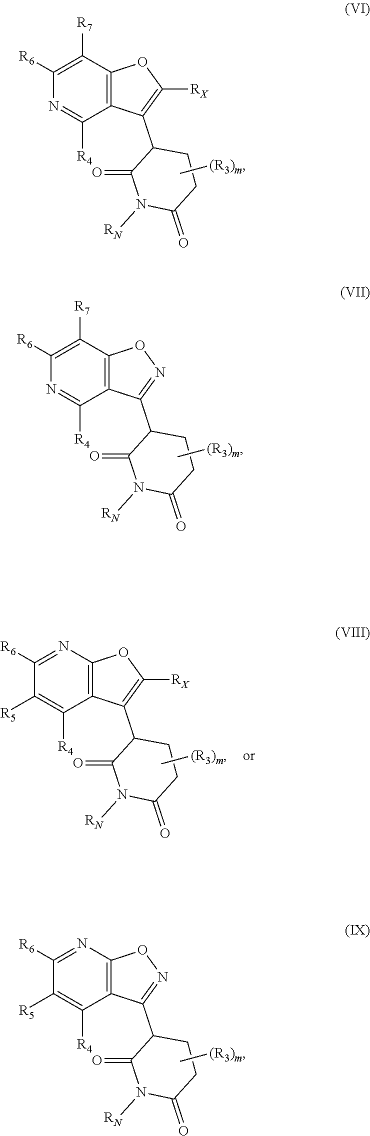

or a pharmaceutically acceptable salt, solvate, prodrug, stereoisomer, or tautomer thereof.

In some embodiments, a compound of Formula I is of Formula II or III:

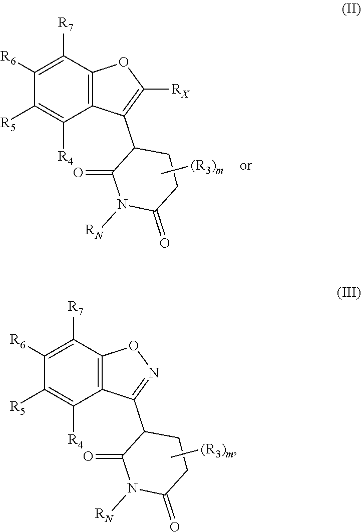

or a pharmaceutically acceptable salt, solvate, prodrug, stereoisomer, or tautomer thereof.

In some embodiments, a compound of Formula I is of Formula Ia:

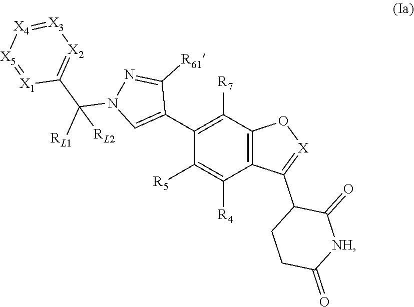

or a pharmaceutically acceptable salt, solvate, prodrug, stereoisomer, or tautomer thereof, wherein:
X is N or CR_{X};
R_{X}, R₄, R₅, and R₇ each independently is H, C₁-C₆ alkyl, C₁-C₆ haloalkyl, or halogen; R₆₁′ is
   H,
   C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
   C₁-C₆ alkoxy, C₁-C₆ haloalkoxy, C₁-C₆ alkyl amino, di-C₁-C₆ alkyl amino, OH, amino, NO₂, halogen, CN,
   C(O)R₆₂, C(O)OR₆₂, or C(O)NR₆₃R₆₄;
R₆₂, R₆₃, and R₆₄ each independently is H, C₁-C₆ alkyl, or C₁-C₆ haloalkyl;
R_{L1} and R_{L2} each independently is H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
X₁ is N or CR_{X1};
X₂ is N or CR_{X2};
X₃ is N or CR_{X3};
X₄ is N or CR_{X4};
X₅ is N or CR_{X5}, provided that no more than four of X₁, X₂, X₃, X₄, and X₅ are N;
R_{X1}, R_{X2}, R_{X3}, R_{X4}, and R_{X5} each independently is H, C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein any two of R_{X1}, R_{X2}, R_{X3}, R_{X4}, and R_{X5} attached to adjacent carbon atoms, together with the carbon atoms to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in any of R_{X1}, R_{X2}, R_{X3}, R_{X4}, and R_{X5} may be replaced with one or more deuterium atoms;
each R_{A} is independently halogen, OH, C₁-C₆ alkyl, C₁-C₆ alkoxy, C₁-C₆ alkyl amino, or di-C₁-C₆ alkyl amino.

In some embodiments, a compound of Formula I is of Formula Ib:

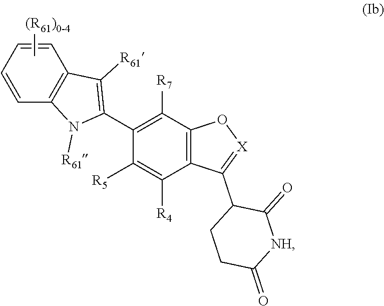

or a pharmaceutically acceptable salt, solvate, prodrug, stereoisomer, or tautomer thereof, wherein:
X is N or CR_{X};
R_{X}, R₄, R₅, and R₇ each independently is H, C₁-C₆ alkyl, C₁-C₆ haloalkyl, or halogen;
each R₆₁″ is independently
   H,
   C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
   C₁-C₆ alkoxy, C₁-C₆ haloalkoxy, C₁-C₆ alkyl amino, di-C₁-C₆ alkyl amino, OH, amino, NO₂, halogen, CN,
   C(O)R₆₂, C(O)OR₆₂, C(O)NR₆₃R₆₄,
   L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
      C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms;
each R₆₁ is independently
   C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
   C₁-C₆ alkoxy, C₁-C₆ haloalkoxy, C₁-C₆ alkyl amino, di-C₁-C₆ alkyl amino, OH, amino, NO₂, halogen, CN, ═O,
   C(O)R₆₂, C(O)OR₆₂, C(O)NR₆₃R₆₄,
   L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
      C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁-2NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms,
wherein:
   L is absent, or a linker selected from O, (CR_{L1}R_{L2})ₙ, O—(CR_{L1}R_{L2})ₙ, (CR_{L1}R_{L2})ₙ—O, and S(O)₂;
   each R_{L1} and each R_{L2} is independently H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
   each R_{A} is independently halogen, OH, C₁-C₆ alkyl, C₁-C₆ alkoxy, C₁-C₆ alkyl amino, or di-C₁-C₆ alkyl amino; and
   n is 1, 2, or 3; or
two R₆₁, together with the carbon atom(s) to which they are attached, may form C₄-C₁₀ carbocyclyl, heterocyclyl comprising one 4- to 6-membered and 1 to 3 heteroatoms selected from N, O, and S, spiro- or fused-heterocyclyl comprising two 4- to 6-membered rings comprising 1 to 4 heteroatoms selected from N, O, and S, or C₆ aryl, wherein the carbocyclyl, heterocyclyl, spiro- or fused-heterocyclyl, or aryl is optionally substituted with one or more groups independently selected from C₁-C₆ alkyl, C₁-C₆ haloalkyl, C₁-C₆ alkoxy, C₁-C₆ haloalkoxy, C₁-C₆ alkyl amino, di-C₁-C₆ alkyl amino, OH, amino, halogen, CN, and ═O; and
each R₆₂, each R₆₃, and each R₆₄ is independently H, C₁-C₆ alkyl, or C₁-C₆ haloalkyl.

For the formulae above, where applicable, embodiments of the variables (e.g., X, Y₄, Y₅, Y₇, R_{X}, R₄, R₅, R₇) in any of the formulae described herein (e.g., Formulae I-IX, Formula Ia, or Formula Ib) are described below.

In some embodiments, X is N.

In some embodiments, X is CR_{X}.

In some embodiments, Y₄ is N.

In some embodiments, Y₄ is CR₄.

In some embodiments, Y₅ is N.

In some embodiments, Y₅ is CR₅.

In some embodiments, Y₇ is N.

In some embodiments, Y₇ is CR₇.

In some embodiments, X is CR_{X}, Y₄ is CR₄, Y₅ is CR₅, and Y₇ is CR₇.

In some embodiments, X is CR_{X}, Y₄ is N, Y₅ is CR₅, and Y₇ is CR₇.

In some embodiments, X is CR_{X}, Y₄ is CR₄, Y₅ is N, and Y₇ is CR₇.

In some embodiments, X is CR_{X}, Y₄ is CR₄, Y₅ is CR₅, and Y₇ is N.

In some embodiments, X is N, Y₄ is CR₄, Y₅ is CR₅, and Y₇ is CR₇.

In some embodiments, X is N, Y₄ is N, Y₅ is CR₅, and Y₇ is CR₇.

In some embodiments, X is N, Y₄ is CR₄, Y₅ is N, and Y₇ is CR₇.

In some embodiments, X is N, Y₄ is CR₄, Y₅ is CR₅, and Y₇ is N.

In some embodiments, R_{X} is H.

In some embodiments, R_{X} is C₁-C₆ alkyl (e.g., methyl, ethyl, propyl, i-propyl, n-butyl, i-butyl, s-butyl, t-butyl, pentyl, or hexyl).

In some embodiments, R_{X} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X} is halogen (e.g., F, Cl, Br, or I).

In some embodiments, R_{X} is F or C₁.

In some embodiments, R₄ is H.

In some embodiments, R₄ is C₁-C₆ alkyl (e.g., methyl, ethyl, propyl, i-propyl, n-butyl, i-butyl, s-butyl, t-butyl, pentyl, or hexyl).

In some embodiments, R₄ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R₄ is halogen (e.g., F, Cl, Br, or I).

In some embodiments, R₄ is F or C₁.

In some embodiments, R₅ is H.

In some embodiments, R₅ is C₁-C₆ alkyl (e.g., methyl, ethyl, propyl, i-propyl, n-butyl, i-butyl, s-butyl, t-butyl, pentyl, or hexyl).

In some embodiments, R₅ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R₅ is halogen (e.g., F, Cl, Br, or I).

In some embodiments, R₅ is F or C₁.

In some embodiments, R₇ is H.

In some embodiments, R₇ is C₁-C₆ alkyl (e.g., methyl, ethyl, propyl, i-propyl, n-butyl, i-butyl, s-butyl, t-butyl, pentyl, or hexyl).

In some embodiments, R₇ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R₇ is halogen (e.g., F, Cl, Br, or I).

In some embodiments, R₇ is F or C₁.

In some embodiments, m is 0.

In some embodiments, m is 1, 2, 3, or 4.

In some embodiments, m is 1.

In some embodiments, at least one R₃ is C₁-C₆ alkyl (e.g., methyl, ethyl, propyl, i-propyl, n-butyl, i-butyl, s-butyl, 1-butyl, pentyl, or hexyl).

In some embodiments, at least one R₃ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₃ is C₁-C₆ alkoxy (e.g., methoxy, ethoxy, propoxy, i-propoxy, n-butoxy, i-butoxy, s-butoxy, t-butoxy, pentoxy, or hexoxy).

In some embodiments, at least one R₃ is C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)).

In some embodiments, at least one R₃ is OH.

In some embodiments, at least one R₃ is halogen (e.g., F, Cl, Br, or I).

In some embodiments, at least one R₃ is F or C₁.

In some embodiments, R_{X} is H.

In some embodiments, R_{X} is C₁-C₆ alkyl (e.g., methyl, ethyl, propyl, i-propyl, n-butyl, i-butyl, s-butyl, t-butyl, pentyl, or hexyl).

In some embodiments, R₆ is R₆′.

In some embodiments, R₆ is (CH₂)₁₋₃—R₆′.

In some embodiments, R₆ is (CH₂)—R₆′.

In some embodiments, R₆ is (CH₂)₂—R₆′.

In some embodiments, R₆ is (CH₂)₃—R₆′.

In some embodiments, R₆′ is heterocyclyl comprising a 4- to 6-membered ring comprising a nitrogen atom and optionally additional 1 to 3 heteroatoms selected from N, O, and S (e.g., heterocyclyl described herein), optionally substituted with one or more R₆₁.

In some embodiments, R₆′ is heterocyclyl comprising a 4-membered ring comprising a nitrogen atom and optionally additional 1 to 3 heteroatoms selected from N, O, and S (e.g., 4-membered heterocyclyl described herein), optionally substituted with one or more R₆₁.

In some embodiments, R₆′ is heterocyclyl comprising a 5-membered ring comprising a nitrogen atom and optionally additional 1 to 3 heteroatoms selected from N, O, and S (e.g., 5-membered heterocyclyl described herein), optionally substituted with one or more R₆₁.

In some embodiments, R₆′ is heterocyclyl comprising a 6-membered ring comprising a nitrogen atom and optionally additional 1 to 3 heteroatoms selected from N, O, and S (e.g., 6-membered heterocyclyl described herein), optionally substituted with one or more R₆₁.

In some embodiments, R₆′ is C₆-C₁₀ aryl (e.g., phenyl or naphthyl), optionally substituted with one or more R₆₁.

In some embodiments, R₆′ is phenyl, optionally substituted with one or more R₆₁.

In some embodiments, R₆′ is heteroaryl comprising a 5- or 6-membered ring comprising a nitrogen atom and optionally additional 1 to 3 heteroatoms selected from N, O, and S (e.g., heteroaryl described herein), optionally substituted with one or more R₆₁.

In some embodiments, R₆′ is heteroaryl comprising a 5-membered ring comprising a nitrogen atom and optionally additional 1 to 3 heteroatoms selected from N, O, and S (e.g., 5-membered heteroaryl described herein), optionally substituted with one or more R₆₁.

In some embodiments, R₆′ is heteroaryl comprising a 5-membered ring comprising a nitrogen atom and optionally additional 1 heteroatom selected from N, O, and S (e.g., 5-membered heteroaryl described herein), optionally substituted with one or more R₆₁.

In some embodiments, R₆′ is heteroaryl comprising a 5-membered ring comprising a nitrogen atom and optionally additional 1 N atom (e.g., 5-membered heteroaryl described herein), optionally substituted with one or more R₆₁.

In some embodiments, R₆′ is heteroaryl comprising a 6-membered ring comprising a nitrogen atom and optionally additional 1 to 3 heteroatoms selected from N, O, and S (e.g., 6-membered heteroaryl described herein), optionally substituted with one or more R₆₁.

In some embodiments, R₆′ is fused-ring heteroaryl comprising a 5- or 6-membered ring comprising a nitrogen atom and optionally additional 1 to 3 heteroatoms selected from N, O, and S and a 5- or 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 to 3 heteroatoms selected from N, O, and S and a 5-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and a 5-membered ring comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and a 5-membered ring comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 heteroatom selected from N, O, and S and a 5-membered ring comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 heteroatom selected from N, O, and S and a 5-membered ring comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 to 3 heteroatoms selected from N, O, and S and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and a 6-membered ring optionally comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and a 6-membered ring (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 heteroatom selected from N, O, and S and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 heteroatom selected from N, O, and S and a 6-membered ring optionally comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 5-membered ring comprising a nitrogen atom and optionally additional 1 heteroatom selected from N, O, and S and a 6-membered ring (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 to 3 heteroatoms selected from N, O, and S and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and a 6-membered ring optionally comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and a 6-membered ring (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 heteroatom selected from N, O, and S and a 6-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 heteroatom selected from N, O, and S and a 6-membered ring optionally comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 heteroatom selected from N, O, and S and a 6-membered ring (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 to 3 heteroatoms selected from N, O, and S and a 5-membered ring optionally comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and a 5-membered ring comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and a 5-membered ring comprising 1 to 2 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 heteroatom selected from N, O, and S and a 5-membered ring comprising 1 to 4 heteroatoms selected from N, O, and S (e.g., fused-ring heteroaryl described herein), optionally substituted with one or more R₆₁.

In some embodiments, R₆′ is fused-ring heteroaryl comprising a 6-membered ring comprising a nitrogen atom and optionally additional 1 heteroatom selected from N, O, and S and a 5-membered ring comprising 1 to 2 heteroatoms selected from N, O, and S (e.g., fused-ring heteroaryl described herein), optionally substituted with one or more R₆₁.

In some embodiments, at least one R₆₁ is C₁-C₆ alkyl (e.g., methyl, ethyl, propyl, i-propyl, n-butyl, i-butyl, s-butyl, t-butyl, pentyl, or hexyl).

In some embodiments, at least one R₆₁ is C₁-C₆ alkyl (e.g., methyl, ethyl, propyl, i-propyl, n-butyl, i-butyl, s-butyl, t-butyl, pentyl, or hexyl), substituted with one or more groups independently selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), halogen (e.g., F, Cl, Br, or I), OH, amino, C₆-C₁₀ aryl, and heteroaryl comprising one or two 5- or 6-membered rings and 1 to 4 heteroatoms selected from N, O, and S (e.g., heteroaryl described herein).

In some embodiments, at least one R₆₁ is C₁-C₆ alkoxy (e.g., methoxy, ethoxy, propoxy, i-propoxy, n-butoxy, i-butoxy, s-butoxy, t-butoxy, pentoxy, or hexoxy).

In some embodiments, at least one R₆₁ is C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)).

In some embodiments, at least one R₆₁ is OH.

In some embodiments, at least one R₆₁ is halogen (e.g., F, Cl, Br, or I).

In some embodiments, at least one R₆₁ is F or C₁.

In some embodiments, at least one R₆₁ is C₁-C₆ alkyl amino (wherein the alkyl is methyl, ethyl, propyl, i-propyl, n-butyl, i-butyl, s-butyl, t-butyl, pentyl, or hexyl), or di-C₁-C₆ alkyl amino (wherein each alkyl is independently methyl, ethyl, propyl, i-propyl, n-butyl, i-butyl, s-butyl, t-butyl, pentyl, or hexyl).

In some embodiments, at least one R₆₁ is OH, amino, NO₂, halogen, or CN.

In some embodiments, at least one R₆₁ is ═O.

In some embodiments, at least one R₆₁ is C(O)R₆₂, C(O)OR₆₂, or C(O)NR₆₃R₆₄.

In some embodiments, at least one Roi is L-C₆-C₁₀ aryl (e.g., phenyl or naphthyl), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{N})C(O)R₆₂, N(R_{N})C(O)OR₆₂, N(R_{X})S(O)₂—C₁-C₆ alkyl, N(R_{X})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁ is L-C₆-C₁₀ aryl (e.g., phenyl or naphthyl) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁ is L-C₆-C₁₀ aryl (e.g., phenyl or naphthyl) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, 1-butoxy, pentoxy, or hexoxy).

In some embodiments, at least one Roi is L-C₆-C₁₀ aryl (e.g., phenyl or naphthyl) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁ is L-C₆-C₁₀ aryl (e.g., phenyl or naphthyl) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-C₆-C₁₀ aryl (e.g., phenyl or naphthyl) substituted with at least one O—(CH₂)₀₋₃—C₆-C₁₀ aryl (e.g., phenyl or naphthyl).

In some embodiments, at least one Roi is L-C₆-C₁₀ aryl (e.g., phenyl or naphthyl) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁ is L-C₆-C₁₀ aryl (e.g., phenyl or naphthyl) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-C₆-C₁₀ aryl (e.g., phenyl or naphthyl) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-C₆-C₁₀ aryl (e.g., phenyl or naphthyl) substituted with at least one OH.

In some embodiments, at least one R₆₁ is L-C₆-C₁₀ aryl (e.g., phenyl or naphthyl) substituted with at least one amino.

In some embodiments, at least one R₆₁ is L-C₆-C₁₀ aryl (e.g., phenyl or naphthyl) substituted with at least one NO₂.

In some embodiments, at least one R₆₁ is L-C₆-C₁₀ aryl (e.g., phenyl or naphthyl) substituted with at least one halogen (e.g., F, Cl, Br, or I).

In some embodiments, at least one R₆₁ is L-C₆-C₁₀ aryl (e.g., phenyl or naphthyl) substituted with at least one CN.

In some embodiments, at least one R₆₁ is L-C₆-C₁₀ aryl (e.g., phenyl or naphthyl) substituted with at least one ═O.

In some embodiments, at least one R₆₁ is L-C₆-C₁₀ aryl (e.g., phenyl or naphthyl) substituted with at least one N(R_{N})C(O)R₆₂.

In some embodiments, at least one R₆₁ is L-C₆-C₁₀ aryl (e.g., phenyl or naphthyl) substituted with at least one N(R_{N})C(O)OR₆₂.

In some embodiments, at least one R₆₁ is L-C₆-C₁₀ aryl (e.g., phenyl or naphthyl) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one Roi is L-C₆-C₁₀ aryl (e.g., phenyl or naphthyl) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁ is L-C₆-C₁₀ aryl (e.g., phenyl or naphthyl) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-C₆-C₁₀ aryl (e.g., phenyl or naphthyl) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁ is L-C₆-C₁₀ aryl (e.g., phenyl or naphthyl) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-C₆-C₁₀ aryl (e.g., phenyl or naphthyl) substituted with at least one C(O)R₆₂.

In some embodiments, at least one R₆₁ is L-C₆-C₁₀ aryl (e.g., phenyl or naphthyl) substituted with at least one C(O)OR₆₂.

In some embodiments, at least one R₆₁ is L-C₆-C₁₀ aryl (e.g., phenyl or naphthyl) substituted with at least one C(O)NR₆₃R₆₄.

In some embodiments, at least one R₆₁ is L-C₆-C₁₀ aryl (e.g., phenyl or naphthyl) substituted with at least one P(═O) (OR₆₂)₂.

In some embodiments, at least one R₆₁ is L-C₆-C₁₀ aryl (e.g., phenyl or naphthyl) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁ is L-C₆-C₁₀ aryl (e.g., phenyl or naphthyl) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-C₆-C₁₀ aryl (e.g., phenyl or naphthyl) substituted with at least one C₆-C₁₀ aryl (e.g., phenyl or naphthyl).

In some embodiments, at least one Roi is L-C₆-C₁₀ aryl (e.g., phenyl or naphthyl) substituted with at least one heteroaryl comprising one or two 5- or 6-membered rings and 1 to 4 heteroatoms selected from N, O, and S (e.g., heteroaryl described herein).

In some embodiments, at least one R₆₁ is L-C₆-C₁₀ aryl (e.g., phenyl or naphthyl), wherein the aryl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one Roi is L-C₆-C₁₀ aryl (e.g., phenyl or naphthyl), wherein the aryl is substituted and one or more hydrogen atoms in the substituent group may be replaced with one or more deuterium atoms.

In some embodiments, at least one R₆₁ is L-heteroaryl comprising one or two 5- or 6-membered rings and 1 to 4 heteroatoms selected from N, O, and S (e.g., heteroaryl described herein), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{X})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁ is L-heteroaryl comprising one or two 5- or 6-membered rings and 1 to 4 heteroatoms selected from N, O, and S (e.g., heteroaryl described herein) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heteroaryl comprising one or two 5- or 6-membered rings and 1 to 4 heteroatoms selected from N, O, and S (e.g., heteroaryl described herein) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁ is L-heteroaryl comprising one or two 5- or 6-membered rings and 1 to 4 heteroatoms selected from N, O, and S (e.g., heteroaryl described herein) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-heteroaryl comprising one or two 5- or 6-membered rings and 1 to 4 heteroatoms selected from N, O, and S (e.g., heteroaryl described herein) substituted with at least one O—(CH₂)₀₋₃—C₆-C₁₀ aryl (e.g., phenyl or naphthyl).

In some embodiments, at least one R₆₁ is L-heteroaryl comprising one or two 5- or 6-membered rings and 1 to 4 heteroatoms selected from N, O, and S (e.g., heteroaryl described herein) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁ is L-heteroaryl comprising one or two 5- or 6-membered rings and 1 to 4 heteroatoms selected from N, O, and S (e.g., heteroaryl described herein) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heteroaryl comprising one or two 5- or 6-membered rings and 1 to 4 heteroatoms selected from N, O, and S (e.g., heteroaryl described herein) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, 1-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heteroaryl comprising one or two 5- or 6-membered rings and 1 to 4 heteroatoms selected from N, O, and S (e.g., heteroaryl described herein) substituted with at least one OH.

In some embodiments, at least one R₆₁ is L-heteroaryl comprising one or two 5- or 6-membered rings and 1 to 4 heteroatoms selected from N, O, and S (e.g., heteroaryl described herein) substituted with at least one amino.

In some embodiments, at least one R₆₁ is L-heteroaryl comprising one or two 5- or 6-membered rings and 1 to 4 heteroatoms selected from N, O, and S (e.g., heteroaryl described herein) substituted with at least one NO₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one halogen (e.g., F, Cl, Br, or I).

In some embodiments, at least one R₆₁ is L-heteroaryl comprising one or two 5- or 6-membered rings and 1 to 4 heteroatoms selected from N, O, and S (e.g., heteroaryl described herein) substituted with at least one CN.

In some embodiments, at least one R₆₁ is L-heteroaryl comprising one or two 5- or 6-membered rings and 1 to 4 heteroatoms selected from N, O, and S (e.g., heteroaryl described herein) substituted with at least one ═O.

In some embodiments, at least one R₆₁ is L-heteroaryl comprising one or two 5- or 6-membered rings and 1 to 4 heteroatoms selected from N, O, and S (e.g., heteroaryl described herein) substituted with at least one N(R_{N})C(O)R₆₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one N(R_{N})C(O)OR₆₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-heteroaryl comprising one or two 5- or 6-membered rings and 1 to 4 heteroatoms selected from N, O, and S (e.g., heteroaryl described herein) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁ is L-heteroaryl comprising one or two 5- or 6-membered rings and 1 to 4 heteroatoms selected from N, O, and S (e.g., heteroaryl described herein) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-heteroaryl comprising one or two 5- or 6-membered rings and 1 to 4 heteroatoms selected from N, O, and S (e.g., heteroaryl described herein) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁ is L-heteroaryl comprising one or two 5- or 6-membered rings and 1 to 4 heteroatoms selected from N, O, and S (e.g., heteroaryl described herein) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C(O)R₆₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C(O)OR₆₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C(O)NR₆₃R₆₄.

In some embodiments, at least one R₆₁ is L-heteroaryl comprising one or two 5- or 6-membered rings and 1 to 4 heteroatoms selected from N, O, and S (e.g., heteroaryl described herein) substituted with at least one P(═O) (OR₆₂)₂.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁ is L-heteroaryl comprising one or two 5- or 6-membered rings and 1 to 4 heteroatoms selected from N, O, and S (e.g., heteroaryl described herein) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-heteroaryl comprising one or two 5- or 6-membered rings and 1 to 4 heteroatoms selected from N, O, and S (e.g., heteroaryl described herein) substituted with at least one C₆-C₁₀ aryl (e.g., phenyl or naphthyl).

In some embodiments, at least one R₆₁ is L-heteroaryl comprising one or two 5- or 6-membered rings and 1 to 4 heteroatoms selected from N, O, and S (e.g., heteroaryl described herein) substituted with at least one heteroaryl comprising one or two 5- or 6-membered rings and 1 to 4 heteroatoms selected from N, O, and S (e.g., heteroaryl described herein).

In some embodiments, at least one R₆₁ is L-heteroaryl comprising one or two 5- or 6-membered rings and 1 to 4 heteroatoms selected from N, O, and S (e.g., heteroaryl described herein), wherein the heteroaryl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁ is L-heteroaryl comprising one or two 5- or 6-membered rings and 1 to 4 heteroatoms selected from N, O, and S (e.g., heteroaryl described herein), wherein the heteroaryl is substituted and one or more hydrogen atoms in the substituent group may be replaced with one or more deuterium atoms.

In some embodiments, at least one R₆₁ is L-C₃-C₆ carbocyclyl (e.g., cyclopropyl, cyclobutyl, cyclopentyl, or cyclohexyl), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{X})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁ is L-C₃-C₆ carbocyclyl (e.g., cyclopropyl, cyclobutyl, cyclopentyl, or cyclohexyl) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁ is L-C₃-C₆ carbocyclyl (e.g., cyclopropyl, cyclobutyl, cyclopentyl, or cyclohexyl) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-C₃-C₆ carbocyclyl (e.g., cyclopropyl, cyclobutyl, cyclopentyl, or cyclohexyl) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁ is L-C₃-C₆ carbocyclyl (e.g., cyclopropyl, cyclobutyl, cyclopentyl, or cyclohexyl) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one Roi is L-C₃-C₆ carbocyclyl (e.g., cyclopropyl, cyclobutyl, cyclopentyl, or cyclohexyl) substituted with at least one O—(CH₂)₀₋₃—C₆-C₁₀ aryl (e.g., phenyl or naphthyl).

In some embodiments, at least one R₆₁ is L-C₃-C₆ carbocyclyl (e.g., cyclopropyl, cyclobutyl, cyclopentyl, or cyclohexyl) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁ is L-C₃-C₆ carbocyclyl (e.g., cyclopropyl, cyclobutyl, cyclopentyl, or cyclohexyl) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-C₃-C₆ carbocyclyl (e.g., cyclopropyl, cyclobutyl, cyclopentyl, or cyclohexyl) substituted with at least one di-C₁-C₆ alkyl amino (wherein each alkyl is independently methyl, ethyl, propyl, i-propyl, n-butyl, i-butyl, s-butyl, 1-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-C₃-C₆ carbocyclyl (e.g., cyclopropyl, cyclobutyl, cyclopentyl, or cyclohexyl) substituted with at least one OH.

In some embodiments, at least one R₆₁ is L-C₃-C₆ carbocyclyl (e.g., cyclopropyl, cyclobutyl, cyclopentyl, or cyclohexyl) substituted with at least one amino.

In some embodiments, at least one R₆₁ is L-C₃-C₆ carbocyclyl (e.g., cyclopropyl, cyclobutyl, cyclopentyl, or cyclohexyl) substituted with at least one NO₂.

In some embodiments, at least one R₆₁ is L-C₃-C₆ carbocyclyl (e.g., cyclopropyl, cyclobutyl, cyclopentyl, or cyclohexyl) substituted with at least one halogen (e.g., F, Cl, Br, or I).

In some embodiments, at least one R₆₁ is L-C₃-C₆ carbocyclyl (e.g., cyclopropyl, cyclobutyl, cyclopentyl, or cyclohexyl) substituted with at least one CN.

In some embodiments, at least one R₆₁ is L-C₃-C₆ carbocyclyl (e.g., cyclopropyl, cyclobutyl, cyclopentyl, or cyclohexyl) substituted with at least one ═O.

In some embodiments, at least one R₆₁ is L-C₃-C₆ carbocyclyl (e.g., cyclopropyl, cyclobutyl, cyclopentyl, or cyclohexyl) substituted with at least one N(R_{X})C(O)R₆₂.

In some embodiments, at least one R₆₁ is L-C₃-C₆ carbocyclyl (e.g., cyclopropyl, cyclobutyl, cyclopentyl, or cyclohexyl) substituted with at least one N(R_{X})C(O)OR₆₂.

In some embodiments, at least one R₆₁ is L-C₃-C₆ carbocyclyl (e.g., cyclopropyl, cyclobutyl, cyclopentyl, or cyclohexyl) substituted with at least one N(R_{X})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-C₃-C₆ carbocyclyl (e.g., cyclopropyl, cyclobutyl, cyclopentyl, or cyclohexyl) substituted with at least one N(R_{X})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁ is L-C₃-C₆ carbocyclyl (e.g., cyclopropyl, cyclobutyl, cyclopentyl, or cyclohexyl) substituted with at least one S(O)₁-2NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-C₃-C₆ carbocyclyl (e.g., cyclopropyl, cyclobutyl, cyclopentyl, or cyclohexyl) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁ is L-C₃-C₆ carbocyclyl (e.g., cyclopropyl, cyclobutyl, cyclopentyl, or cyclohexyl) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-C₃-C₆ carbocyclyl (e.g., cyclopropyl, cyclobutyl, cyclopentyl, or cyclohexyl) substituted with at least one C(O)R₆₂.

In some embodiments, at least one R₆₁ is L-C₃-C₆ carbocyclyl (e.g., cyclopropyl, cyclobutyl, cyclopentyl, or cyclohexyl) substituted with at least one C(O)OR₆₂.

In some embodiments, at least one R₆₁ is L-C₃-C₆ carbocyclyl (e.g., cyclopropyl, cyclobutyl, cyclopentyl, or cyclohexyl) substituted with at least one C(O)NR₆₃R₆₄.

In some embodiments, at least one R₆₁ is L-C₃-C₆ carbocyclyl (e.g., cyclopropyl, cyclobutyl, cyclopentyl, or cyclohexyl) substituted with at least one P(═O) (OR₆₂)₂.

In some embodiments, at least one R₆₁ is L-C₃-C₆ carbocyclyl (e.g., cyclopropyl, cyclobutyl, cyclopentyl, or cyclohexyl) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁ is L-C₃-C₆ carbocyclyl (e.g., cyclopropyl, cyclobutyl, cyclopentyl, or cyclohexyl) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one Roi is L-C₃-C₆ carbocyclyl (e.g., cyclopropyl, cyclobutyl, cyclopentyl, or cyclohexyl) substituted with at least one C₆-C₁₀ aryl (e.g., phenyl or naphthyl).

In some embodiments, at least one R₆₁ is L-C₃-C₆ carbocyclyl (e.g., cyclopropyl, cyclobutyl, cyclopentyl, or cyclohexyl) substituted with at least one heteroaryl comprising one or two 5- or 6-membered rings and 1 to 4 heteroatoms selected from N, O, and S (e.g., heteroaryl described herein).

In some embodiments, at least one Roi is L-C₃-C₆ carbocyclyl (e.g., cyclopropyl, cyclobutyl, cyclopentyl, or cyclohexyl), wherein the carbocyclyl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁ is L-C₃-C₆ carbocyclyl (e.g., cyclopropyl, cyclobutyl, cyclopentyl, or cyclohexyl), wherein the carbocyclyl is substituted and one or more hydrogen atoms in the substituent group may be replaced with one or more deuterium atoms.

In some embodiments, at least one R₆₁ is L-heterocyclyl comprising one or two 4-to 6-membered rings and 1 to 4 heteroatoms selected from N, O, and S (e.g., heterocyclyl described herein), optionally substituted with one or more groups independently selected from:
C₁-C₆ alkyl (e.g., methyl, ethyl, propyl, i-propyl, n-butyl, i-butyl, s-butyl, 1-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{X})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C—C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, C₁, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one O—(CH₂)₀₋₃—C₆-C₁₀ aryl (e.g., phenyl or naphthyl).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one OH.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one amino.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one NO₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one halogen (e.g., F, Cl, Br, or I).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one CN.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one ═O.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one N(R_{N})C(O)R₆₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one N(R_{N})C(O)OR₆₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one Ro is L-heterocyclyl comprising one or two 4-to 6-membered rings and 1 to 4 heteroatoms selected from N, O, and S (e.g., heterocyclyl described herein) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C(O)R₆₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C(O)OR₆₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C(O)NR₆₃R₆₄.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one P(═O) (OR₆₂)₂.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one C₆-C₁₀ aryl (e.g., phenyl or naphthyl).

In some embodiments, at least one R₆₁ is L-heterocyclyl comprising one or two 4-to 6-membered rings and 1 to 4 heteroatoms selected from N, O, and S (e.g., heterocyclyl described herein) substituted with at least one heteroaryl comprising one or two 5- or 6-membered rings and 1 to 4 heteroatoms selected from N, O, and S (e.g., heteroaryl described herein).

In some embodiments, at least one R₆₁ is L-heterocyclyl comprising one or two 4-to 6-membered rings and 1 to 4 heteroatoms selected from N, O, and S (e.g., heterocyclyl described herein), wherein the heterocyclyl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁ is L-heterocyclyl comprising one or two 4-to 6-membered rings and 1 to 4 heteroatoms selected from N, O, and S (e.g., heterocyclyl described herein), wherein the heterocyclyl is substituted and one or more hydrogen atoms in the substituent group may be replaced with one or more deuterium atoms.

In some embodiments, L is absent.

In some embodiments, L is O.

In some embodiments, L is (CR_{L1}R_{L2})ₙ.

In some embodiments, L is O—(CR_{L1}R_{L2})ₙ.

In some embodiments, L is (CR_{L1}R_{L2})ₙ—O.

In some embodiments, L is S(O)₂.

In some embodiments, n is 1.

In some embodiments, n is 2.

In some embodiments, n is 3.

In some embodiments, R_{L1} and R_{L2} each independently is H, CH₃, CH₂CH₃, or CH(CH₃)₂.

In some embodiments, each R_{L1} and each R_{L2} is H.

In some embodiments, each R_{L1} is H and at least one R_{L2} is CH₃, CH₂CH₃, or CH(CH₃)₂.

In II some embodiments, at least one of R_{L1} is CH₃, CH₂CH₃, or CH(CH₃)₂ and at least one R_{L2} is CH₃, CH₂CH₃, or CH(CH₃)₂.

In some embodiments, R_{L1} and R_{L2}, together with the carbon atom to which they are attached, form C(O).

In some embodiments, R_{L1} and R_{L2}, together with the carbon atom to which they are attached, form C₃-C₆ cycloalkyl (e.g., cyclopropyl, cyclobutyl, cyclopentyl, or cyclohexyl).

In some embodiments, R_{L1} and R_{L2}, together with the carbon atom to which they are attached, form heterocyclyl comprising a 4- to 6-membered ring comprising and 1 to 3 heteroatoms selected from N, O, and S (e.g., heterocyclyl described herein).

In some embodiments, at least one R_{A} is halogen (e.g., F, Cl, Br, or I).

In some embodiments, at least one R_{A} is OH.

In some embodiments, at least one R_{A} is C₁-C₆ alkyl (e.g., methyl, ethyl, propyl, i-propyl, n-butyl, i-butyl, s-butyl, t-butyl, pentyl, or hexyl).

In some embodiments, at least one R_{A} is C₁-C₆ alkoxy (e.g., methoxy, ethoxy, propoxy, i-propoxy, n-butoxy, i-butoxy, s-butoxy, 1-butoxy, pentoxy, or hexoxy).

In some embodiments, at least one R_{A} is C₁-C₆ alkyl amino (wherein the alkyl is methyl, ethyl, propyl, i-propyl, n-butyl, i-butyl, s-butyl, t-butyl, pentyl, or hexyl), or di-C₁-C₆ alkyl amino (wherein each alkyl is independently methyl, ethyl, propyl, i-propyl, n-butyl, i-butyl, s-butyl, 1-butyl, pentyl, or hexyl).

In some embodiments, two R₆₁, together with the carbon atom(s) to which they are attached, form C₄-C₁₀ carbocyclyl (e.g., cyclobutyl, cyclopentyl, cyclohexyl, cycloheptyl, cyclooctyl, cyclononyl, or cyclodecyl), optionally substituted with one or more groups independently selected from C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)), C₁-C₆ alkoxy (e.g., methoxy, ethoxy, propoxy, i-propoxy, n-butoxy, i-butoxy, s-butoxy, t-butoxy, pentoxy, or hexoxy), C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), OH, amino, halogen (e.g., F, Cl, Br, or I), CN, and —O.

In some embodiments, two Rol, together with the carbon atom(s) to which they are attached, form heterocyclyl comprising one 4- to 6-membered and 1 to 3 heteroatoms selected from N, O, and S (e.g., heterocyclyl described herein), optionally substituted with one or more groups independently selected from C₁-C₆ alkyl (e.g., methyl, ethyl, propyl, i-propyl, n-butyl, i-butyl, s-butyl, 1-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)), C₁-C₆ alkoxy (e.g., methoxy, ethoxy, propoxy, i-propoxy, n-butoxy, i-butoxy, s-butoxy, t-butoxy, pentoxy, or hexoxy), C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), OH, amino, halogen (e.g., F, Cl, Br, or I), CN, and ═O.

In some embodiments, two R₆₁, together with the carbon atom(s) to which they are attached, form heterocyclyl comprising one 4-membered and 1 to 3 heteroatoms selected from N, O, and S (e.g., 4-membered heterocyclyl described herein), optionally substituted as described herein.

In some embodiments, two R₆₁, together with the carbon atom(s) to which they are attached, form heterocyclyl comprising one 5-membered and 1 to 3 heteroatoms selected from N, O, and S (e.g., 5-membered heterocyclyl described herein), optionally substituted as described herein.

In some embodiments, two R₆₁, together with the carbon atom(s) to which they are attached, form heterocyclyl comprising one 6-membered and 1 to 3 heteroatoms selected from N, O, and S (e.g., 6-membered heterocyclyl described herein), optionally substituted as described herein.

In some embodiments, two R₆₁, together with the carbon atom(s) to which they are attached, form spiro- or fused-heterocyclyl comprising two 4- to 6-membered rings comprising 1 to 4 heteroatoms selected from N, O, and S (e.g., spiro- or fused-heterocyclyl described herein), optionally substituted with one or more groups independently selected from C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)), C₁-C₆ alkoxy (e.g., methoxy, ethoxy, propoxy, i-propoxy, n-butoxy, i-butoxy, s-butoxy, t-butoxy, pentoxy, or hexoxy), C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), OH, amino, halogen (e.g., F, Cl, Br, or I), CN, and ═O.

In some embodiments, two R₆₁, together with the carbon atom(s) to which they are attached, form spiro- or fused-heterocyclyl comprising two 5- to 6-membered rings comprising 1 to 4 heteroatoms selected from N, O, and S (e.g., spiro- or fused-heterocyclyl described herein), optionally substituted as described herein.

In some embodiments, two R₆₁, together with the carbon atom(s) to which they are attached, form C₆ aryl, optionally substituted with one or more groups independently selected from C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)), C₁-C₆ alkoxy (e.g., methoxy, ethoxy, propoxy, i-propoxy, n-butoxy, i-butoxy, s-butoxy, t-butoxy, pentoxy, or hexoxy), C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), OH, amino, halogen (e.g., F, Cl, Br, or I), CN, and ═O.

In some embodiments, each R₆₂ is H.

In some embodiments, at least one Roz is C₁-C₆ alkyl (e.g., methyl, ethyl, propyl, i-propyl, n-butyl, i-butyl, s-butyl, t-butyl, pentyl, or hexyl).

In some embodiments, at least one R₆₂ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, each R₆₃ is H.

In some embodiments, at least one R₆₃ is C₁-C₆ alkyl (e.g., methyl, ethyl, propyl, i-propyl, n-butyl, i-butyl, s-butyl, t-butyl, pentyl, or hexyl).

In some embodiments, at least one R₆₃ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, each R₆₄ is H.

In some embodiments, at least one R₆₄ is C₁-C₆ alkyl (e.g., methyl, ethyl, propyl, i-propyl, n-butyl, i-butyl, s-butyl, t-butyl, pentyl, or hexyl).

In some embodiments, at least one R₆₄ is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R₆₁′ is H.

In some embodiments, R₆₁′ is C₁-C₆ alkyl (e.g., methyl, ethyl, propyl, i-propyl, n-butyl, i-butyl, s-butyl, t-butyl, pentyl, or hexyl).

In some embodiments, Roj′ is C₁-C₆ alkyl (e.g., methyl, ethyl, propyl, i-propyl, n-butyl, i-butyl, s-butyl, t-butyl, pentyl, or hexyl), substituted with one or more groups independently selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), halogen (e.g., F, Cl, Br, or I), OH, amino, C₆-C₁₀ aryl, and heteroaryl comprising one or two 5- or 6-membered rings and 1 to 4 heteroatoms selected from N, O, and S (e.g., heteroaryl described herein).

In some embodiments, R₆₁′ is C₁-C₆ alkoxy (e.g., methoxy, ethoxy, propoxy, i-propoxy, n-butoxy, i-butoxy, s-butoxy, t-butoxy, pentoxy, or hexoxy).

In some embodiments, R₆₁′ is C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)).

In some embodiments, R₆₁′ is OH.

In some embodiments, R₆₁′ is halogen (e.g., F, Cl, Br, or I).

In some embodiments, R₆₁′ is F or C₁.

In some embodiments, R₆₁′ is C₁-C₆ alkyl amino (wherein the alkyl is methyl, ethyl, propyl, i-propyl, n-butyl, i-butyl, s-butyl, t-butyl, pentyl, or hexyl), or di-C₁-C₆ alkyl amino (wherein each alkyl is independently methyl, ethyl, propyl, i-propyl, n-butyl, i-butyl, s-butyl, t-butyl, pentyl, or hexyl).

In some embodiments, R₆₁′ is OH, amino, NO₂, halogen, or CN.

In some embodiments, R₆₁′ is C(O)R₆₂, C(O)OR₆₂, or C(O)NR₆₃R₆₄.

In some embodiments, R₆₁′ is H or halogen.

In some embodiments, X₁ is N.

In some embodiments, X₁ is CR_{X1}.

In some embodiments, X₂ is N.

In some embodiments, X₂ is CR_{X1}.

In some embodiments, X₃ is N.

In some embodiments, X₃ is CR_{X1}.

In some embodiments, X₄ is N.

In some embodiments, X₄ is CR_{X1}.

In some embodiments, X₅ is N.

In some embodiments, X₅ is CR_{X1}.

In some embodiments, one of X₁, X₂, X₃, X₄, and X₅ is N.

In some embodiments, two of X₁, X₂, X₃, X₄, and X₅ are N.

In some embodiments, three of X₁, X₂, X₃, X₄, and X₅ are N.

In some embodiments, four of X₁, X₂, X₃, X₄, and X₅ are N.

In some embodiments, R_{X1} is H.

In some embodiments, R_{X1} is C₁-C₆ alkyl (e.g., methyl, ethyl, propyl, i-propyl, n-butyl, i-butyl, s-butyl, t-butyl, pentyl, or hexyl).

In some embodiments, R_{X1} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X1} is halogen (e.g., F, Cl, Br, or I).

In some embodiments, R_{X1} is F or C₁.

In some embodiments, R_{X1} is (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, C₁, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy.

In some embodiments, R_{X1} is O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, R_{X1} is O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, R_{X1} is O—(CH₂)₀₋₃—C₆-C₁₀ aryl (e.g., phenyl or naphthyl).

In some embodiments, R_{X1} is O—(CH₂)₀₋₃-heteroaryl comprising one or two 5- or 6-membered rings and 1 to 4 heteroatoms selected from N, O, and S (e.g., heteroaryl described herein).

In some embodiments, R_{X1} is C₁-C₆ alkyl amino (wherein the alkyl is methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), or di-C₁-C₆ alkyl amino (wherein each alkyl is independently methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X1} is OH, amino, NO₂, halogen, CN, or ═O.

In some embodiments, R_{X1} is N(R_{N})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, or S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl and is optionally substituted with one or more R_{A}.

In some embodiments, R_{X1} is C(O)R₆₂, C(O)OR₆₂, or C(O)NR₆₃R₆₄.

In some embodiments, R_{X1} is P(═O) (OR₆₂)₂.

In some embodiments, R_{X1} is C₃-C₆ carbocyclyl (e.g., cyclopropyl, cyclobutyl, cyclopentyl, or cyclohexyl) optionally substituted with one or more R_{A}, heterocyclyl comprising one or two 3- to 6-membered rings and 1 to 4 heteroatoms selected from N, O, and S (e.g., heterocyclyl described herein) optionally substituted with one or more R_{A}, C₆-C₁₀ aryl (e.g., phenyl or naphthyl), or heteroaryl comprising one or two 5- or 6-membered rings and 1 to 4 heteroatoms selected from N, O, and S (e.g., heteroaryl described herein).

In some embodiments, R_{X2} is H.

In some embodiments, R_{X2} is C₁-C₆ alkyl (e.g., methyl, ethyl, propyl, i-propyl, n-butyl, i-butyl, s-butyl, t-butyl, pentyl, or hexyl).

In some embodiments, R_{X2} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X2} is halogen (e.g., F, Cl, Br, or I).

In some embodiments, R_{X2} is F or C₁.

In some embodiments, R_{X2} is (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X2} is O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, R_{X2} is O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, R_{X2} is O—(CH₂)₀₋₃—C₆-C₁₀ aryl (e.g., phenyl or naphthyl).

In some embodiments, R_{X2} is O—(CH₂)₀₋₃-heteroaryl comprising one or two 5- or 6-membered rings and 1 to 4 heteroatoms selected from N, O, and S (e.g., heteroaryl described herein).

In some embodiments, R_{X2} is C₁-C₆ alkyl amino (wherein the alkyl is methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), or di-C₁-C₆ alkyl amino (wherein each alkyl is independently methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X2} is OH, amino, NO₂, halogen, CN, or ═O.

In some embodiments, R_{X2} is N(R_{N})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, or S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl and is optionally substituted with one or more R_{A}.

In some embodiments, R_{X2} is C(O)R₆₂, C(O)OR₆₂, or C(O)NR₆₃R₆₄.

In some embodiments, R_{X2} is P(═O) (OR₆₂)₂.

In some embodiments, R_{X2} is C₃-C₆ carbocyclyl (e.g., cyclopropyl, cyclobutyl, cyclopentyl, or cyclohexyl) optionally substituted with one or more R_{A}, heterocyclyl comprising one or two 3- to 6-membered rings and 1 to 4 heteroatoms selected from N, O, and S (e.g., heterocyclyl described herein) optionally substituted with one or more R_{A}, C₆-C₁₀ aryl (e.g., phenyl or naphthyl), or heteroaryl comprising one or two 5- or 6-membered rings and 1 to 4 heteroatoms selected from N, O, and S (e.g., heteroaryl described herein).

In some embodiments, R_{X3} is H.

In some embodiments, R_{X3} is C₁-C₆ alkyl (e.g., methyl, ethyl, propyl, i-propyl, n-butyl, i-butyl, s-butyl, t-butyl, pentyl, or hexyl).

In some embodiments, R_{X3} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X3} is halogen (e.g., F, Cl, Br, or I).

In some embodiments, R_{X3} is F or C₁.

In some embodiments, R_{X3} is (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X3} is O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, R_{X3} is O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, R_{X3} is O—(CH₂)₀₋₃—C₆-C₁₀ aryl (e.g., phenyl or naphthyl).

In some embodiments, R_{X3} is O—(CH₂)₀₋₃-heteroaryl comprising one or two 5- or 6-membered rings and 1 to 4 heteroatoms selected from N, O, and S (e.g., heteroaryl described herein).

In some embodiments, R_{X3} is C₁-C₆ alkyl amino (wherein the alkyl is methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), or di-C₁-C₆ alkyl amino (wherein each alkyl is independently methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X3} is OH, amino, NO₂, halogen, CN, or ═O.

In some embodiments, R_{X3} is N(R_{N})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, or S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl and is optionally substituted with one or more R_{A}.

In some embodiments, R_{X3} is C(O)R₆₂, C(O)OR₆₂, or C(O)NR₆₃R₆₄.

In some embodiments, R_{X3} is P(—O) (OR₆₂)₂.

In some embodiments, R_{X3} is C₃-C₆ carbocyclyl (e.g., cyclopropyl, cyclobutyl, cyclopentyl, or cyclohexyl) optionally substituted with one or more R_{A}, heterocyclyl comprising one or two 3- to 6-membered rings and 1 to 4 heteroatoms selected from N, O, and S (e.g., heterocyclyl described herein) optionally substituted with one or more R_{A}, C₆-C₁₀ aryl (e.g., phenyl or naphthyl), or heteroaryl comprising one or two 5- or 6-membered rings and 1 to 4 heteroatoms selected from N, O, and S (e.g., heteroaryl described herein).

In some embodiments, R_{X4} is H.

In some embodiments, R_{X4} is C₁-C₆ alkyl (e.g., methyl, ethyl, propyl, i-propyl, n-butyl, i-butyl, s-butyl, t-butyl, pentyl, or hexyl).

In some embodiments, R_{X4} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X4} is halogen (e.g., F, Cl, Br, or I).

In some embodiments, R_{X4} is F or C₁.

In some embodiments, R_{X4} is (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, 1-butoxy, pentoxy, or hexoxy).

In some embodiments, R_{X4} is O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, R_{X4} is O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, R_{X4} is O—(CH₂)₀₋₃—C₆-C₁₀ aryl (e.g., phenyl or naphthyl).

In some embodiments, R_{X4} is O—(CH₂)₀₋₃-heteroaryl comprising one or two 5- or 6-membered rings and 1 to 4 heteroatoms selected from N, O, and S (e.g., heteroaryl described herein).

In some embodiments, R_{X4} is C₁-C₆ alkyl amino (wherein the alkyl is methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), or di-C₁-C₆ alkyl amino (wherein each alkyl is independently methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, 1-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X4} is OH, amino, NO₂, halogen, CN, or ═O.

In some embodiments, R_{X4} is N(R_{N})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, or S(O)₀₋₂—C₁-C₆ alkyl, wherein the C₁-C₆ alkyl is methyl, ethyl, propyl, i-propyl, n-butyl, i-butyl, s-butyl, 1-butyl, pentyl, or hexyl and wherein the C₃-C₆ carbocyclyl is cyclopropyl, cyclobutyl, cyclopentyl, or cyclohexyl and is optionally substituted with one or more R_{A}.

In some embodiments, R_{X4} is C(O)R₆₂, C(O)OR₆₂, or C(O)NR₆₃R₆₄.

In some embodiments, R_{X4} is P(═O) (OR₆₂)₂.

In some embodiments, R_{X4} is C₃-C₆ carbocyclyl (e.g., cyclopropyl, cyclobutyl, cyclopentyl, or cyclohexyl) optionally substituted with one or more R_{A}, heterocyclyl comprising one or two 3- to 6-membered rings and 1 to 4 heteroatoms selected from N, O, and S (e.g., heterocyclyl described herein) optionally substituted with one or more R_{A}, C₆-C₁₀ aryl (e.g., phenyl or naphthyl), or heteroaryl comprising one or two 5- or 6-membered rings and 1 to 4 heteroatoms selected from N, O, and S (e.g., heteroaryl described herein).

In some embodiments, R_{X5} is H.

In some embodiments, R_{X5} is C₁-C₆ alkyl (e.g., methyl, ethyl, propyl, i-propyl, n-butyl, i-butyl, s-butyl, t-butyl, pentyl, or hexyl).

In some embodiments, R_{X5} is C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, R_{X5} is halogen (e.g., F, Cl, Br, or I).

In some embodiments, R_{X5} is For C₁.

In some embodiments, R_{X5} is (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, R_{X5} is O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, R_{X5} is O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, R_{X5} is O—(CH₂)₀₋₃—C₆-C₁₀ aryl (e.g., phenyl or naphthyl).

In some embodiments, R_{X5} is O—(CH₂)₀₋₃-heteroaryl comprising one or two 5- or 6-membered rings and 1 to 4 heteroatoms selected from N, O, and S (e.g., heteroaryl described herein).

In some embodiments, R_{X5} is C₁-C₆ alkyl amino (wherein the alkyl is methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), or di-C₁-C₆ alkyl amino (wherein each alkyl is independently methyl, ethyl, propyl, i-propyl, n-butyl, i-butyl, s-butyl, t-butyl, pentyl, or hexyl) optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, 1-butoxy, pentoxy, or hexoxy).

In some embodiments, R_{X5} is OH, amino, NO₂, halogen, CN, or ═O.

In some embodiments, R_{X5} is N(R_{N})C(O)R₆₂, N(R_{N})C(O)OR₆₂, N(R_{X})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, or S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl and is optionally substituted with one or more R_{A}.

In some embodiments, R_{X5} is C(O)R₆₂, C(O)OR₆₂, or C(O)NR₆₃R₆₄.

In some embodiments, R_{X5} is P(═O) (OR₆₂)₂.

In some embodiments, R_{X5} is C₃-C₆ carbocyclyl (e.g., cyclopropyl, cyclobutyl, cyclopentyl, or cyclohexyl) optionally substituted with one or more R_{A}, heterocyclyl comprising one or two 3- to 6-membered rings and 1 to 4 heteroatoms selected from N, O, and S (e.g., heterocyclyl described herein) optionally substituted with one or more R_{A}, C₆-C₁₀ aryl (e.g., phenyl or naphthyl), or heteroaryl comprising one or two 5- or 6-membered rings and 1 to 4 heteroatoms selected from N, O, and S (e.g., heteroaryl described herein).

In some embodiments, two of R_{X1}, R_{X2}, R_{X3}, R_{X4}, and R_{X5} attached to adjacent carbon atoms, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more halogen, and wherein one or more hydrogen atoms in any of R_{X1}, R_{X2}, R_{X3}, R_{X4}, and R_{X5} may be replaced with one or more deuterium atoms.

In some embodiments, each R₆₁″ is H.

In some embodiments, at least one R₆₁″ is H.

In some embodiments, at least one R₆₁″ is C₁-C₆ alkyl (e.g., methyl, ethyl, propyl, i-propyl, n-butyl, i-butyl, s-butyl, t-butyl, pentyl, or hexyl).

In some embodiments, at least one R₆₁″ is C₁-C₆ alkyl (e.g., methyl, ethyl, propyl, i-propyl, n-butyl, i-butyl, s-butyl, t-butyl, pentyl, or hexyl), substituted with one or more groups independently selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), halogen (e.g., F, Cl, Br, or I), OH, amino, C₆-C₁₀ aryl, and heteroaryl comprising one or two 5- or 6-membered rings and 1 to 4 heteroatoms selected from N, O, and S (e.g., heteroaryl described herein).

In some embodiments, at least one R₆₁″ is C₁-C₆ alkoxy (e.g., methoxy, ethoxy, propoxy, i-propoxy, n-butoxy, i-butoxy, s-butoxy, t-butoxy, pentoxy, or hexoxy).

In some embodiments, at least one R₆₁″ is C₁-C₆ haloalkoxy (e.g., methoxy, ethoxy, propoxy, i-propoxy, n-butoxy, i-butoxy, s-butoxy, t-butoxy, pentoxy, or hexoxy, each substituted with one or more halogen (e.g., F, Cl, Br, or I)).

In some embodiments, at least one R₆₁″ is OH.

In some embodiments, at least one R₆₁″ is halogen (e.g., F, Cl, Br, or I).

In some embodiments, at least one R₆₁″ is F or C₁.

In some embodiments, at least one R₆₁″ is C₁-C₆ alkyl amino (wherein the alkyl is methyl, ethyl, propyl, i-propyl, n-butyl, i-butyl, s-butyl, t-butyl, pentyl, or hexyl), or di-C₁-C₆ alkyl amino (wherein each alkyl is independently methyl, ethyl, propyl, i-propyl, n-butyl, i-butyl, s-butyl, t-butyl, pentyl, or hexyl).

In some embodiments, at least one R₆₁″ is OH, amino, NO₂, halogen, or CN.

In some embodiments, at least one R₆₁″ is C(O)R₆₂, C(O)OR₆₂, or C(O)NR₆₃R₆₄.

In some embodiments, at least one R₆₁″ is L-C₆-C₁₀ aryl (e.g., phenyl or naphthyl), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, cyclobutyl, cyclopentyl, or cyclohexyl, heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁″ is L-C₆-C₁₀ aryl (e.g., phenyl or naphthyl) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁″ is L-C₆-C₁₀ aryl (e.g., phenyl or naphthyl) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-C₆-C₁₀ aryl (e.g., phenyl or naphthyl) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁″ is L-C₆-C₁₀ aryl (e.g., phenyl or naphthyl) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-C₆-C₁₀ aryl (e.g., phenyl or naphthyl) substituted with at least one O—(CH₂)₀₋₃—C₆-C₁₀ aryl (e.g., phenyl or naphthyl).

In some embodiments, at least one R₆₁″ is L-C₆-C₁₀ aryl (e.g., phenyl or naphthyl) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁″ is L-C₆-C₁₀ aryl (e.g., phenyl or naphthyl) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-C₆-C₁₀ aryl (e.g., phenyl or naphthyl) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-C₆-C₁₀ aryl (e.g., phenyl or naphthyl) substituted with at least one OH.

In some embodiments, at least one R₆₁″ is L-C₆-C₁₀ aryl (e.g., phenyl or naphthyl) substituted with at least one amino.

In some embodiments, at least one R₆₁″ is L-C₆-C₁₀ aryl (e.g., phenyl or naphthyl) substituted with at least one NO₂.

In some embodiments, at least one R₆₁″ is L-C₆-C₁₀ aryl (e.g., phenyl or naphthyl) substituted with at least one halogen (e.g., F, Cl, Br, or I).

In some embodiments, at least one R₆₁″ is L-C₆-C₁₀ aryl (e.g., phenyl or naphthyl) substituted with at least once CN.

In some embodiments, at least one R₆₁″ is L-C₆-C₁₀ aryl (e.g., phenyl or naphthyl) substituted with at least one ═O.

In some embodiments, at least one R₆₁″ is L-C₆-C₁₀ aryl (e.g., phenyl or naphthyl) substituted with at least one N(R_{X})C(O)R₆₂.

In some embodiments, at least one R₆₁″ is L-C₆-C₁₀ aryl (e.g., phenyl or naphthyl) substituted with at least one N(R)C(O)OR₆₂.

In some embodiments, at least one R₆₁″ is L-C₆-C₁₀ aryl (e.g., phenyl or naphthyl) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₆-C₁₀ aryl (e.g., phenyl or naphthyl) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁″ is L-C₆-C₁₀ aryl (e.g., phenyl or naphthyl) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₆-C₁₀ aryl (e.g., phenyl or naphthyl) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁″ is L-C₆-C₁₀ aryl (e.g., phenyl or naphthyl) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₆-C₁₀ aryl (e.g., phenyl or naphthyl) substituted with at least one C(O)R₆₂.

In some embodiments, at least one R₆₁″ is L-C₆-C₁₀ aryl (e.g., phenyl or naphthyl) substituted with at least one C(O)OR₆₂.

In some embodiments, at least one R₆₁″ is L-C₆-C₁₀ aryl (e.g., phenyl or naphthyl) substituted with at least one C(O)NR₆₃R₆₄.

In some embodiments, at least one R₆₁″ is L-C₆-C₁₀ aryl (e.g., phenyl or naphthyl) substituted with at least one P(═O) (OR₆₂)₂.

In some embodiments, at least one R₆₁″ is L-C₆-C₁₀ aryl (e.g., phenyl or naphthyl) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁″ is L-C₆-C₁₀ aryl (e.g., phenyl or naphthyl) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-C₆-C₁₀ aryl (e.g., phenyl or naphthyl) substituted with at least one C₆-C₁₀ aryl (e.g., phenyl or naphthyl).

In some embodiments, at least one R₆₁″ is L-C₆-C₁₀ aryl (e.g., phenyl or naphthyl) substituted with at least one heteroaryl comprising one or two 5- or 6-membered rings and 1 to 4 heteroatoms selected from N, O, and S (e.g., heteroaryl described herein).

In some embodiments, at least one R₆₁″ is L-C₆-C₁₀ aryl (e.g., phenyl or naphthyl), wherein the aryl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁″ is L-C₆-C₁₀ aryl (e.g., phenyl or naphthyl), wherein the aryl is substituted and one or more hydrogen atoms in the substituent group may be replaced with one or more deuterium atoms.

In some embodiments, at least one R₆₁″ is L-heteroaryl comprising one or two 5- or 6-membered rings and 1 to 4 heteroatoms selected from N, O, and S (e.g., heteroaryl described herein), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C—C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one O—(CH₂)₀₋₃—C₆-C₁₀ aryl (e.g., phenyl or naphthyl).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one di-C₁-C₆ alkyl amino (wherein each alkyl is independently methyl, ethyl, propyl, i-propyl, n-butyl, i-butyl, s-butyl, 1-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one OH.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one amino.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one NO₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one halogen (e.g., F, Cl, Br, or I).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one CN.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one ═O.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one N(R_{N})C(O)R₆₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one N(R_{N})C(O)OR₆₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C(O)R₆₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C(O)OR₆₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C(O)NR₆₃R₆₄.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one P(═O) (OR₆₂)₂.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-heteroaryl comprising one or two 5- or 6-membered rings and 1 to 4 heteroatoms selected from N, O, and S (e.g., heteroaryl described herein) substituted with at least one C₆-C₁₀ aryl (e.g., phenyl or naphthyl).

In some embodiments, at least one R₆₁″ is L-heteroaryl comprising one or two 5- or 6-membered rings and 1 to 4 heteroatoms selected from N, O, and S (e.g., heteroaryl described herein) substituted with at least one heteroaryl comprising one or two 5- or 6-membered rings and 1 to 4 heteroatoms selected from N, O, and S (e.g., heteroaryl described herein).

In some embodiments, at least one R₆₁″ is L-heteroaryl comprising one or two 5- or 6-membered rings and 1 to 4 heteroatoms selected from N, O, and S (e.g., heteroaryl described herein), wherein the heteroaryl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁″ is L-heteroaryl comprising one or two 5- or 6-membered rings and 1 to 4 heteroatoms selected from N, O, and S (e.g., heteroaryl described herein), wherein the heteroaryl is substituted and one or more hydrogen atoms in the substituent group may be replaced with one or more deuterium atoms.

In some embodiments, at least one R₆₁″ is L-C₃-C₆ carbocyclyl (e.g., cyclopropyl, cyclobutyl, cyclopentyl, or cyclohexyl), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂,
C₃-C₆ carbocyclyl (e.g., cyclopropyl, cyclobutyl, cyclopentyl, or cyclohexyl), heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein),
wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁″ is L-C₃-C₆ carbocyclyl (e.g., cyclopropyl, cyclobutyl, cyclopentyl, or cyclohexyl) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁″ is L-C₃-C₆ carbocyclyl (e.g., cyclopropyl, cyclobutyl, cyclopentyl, or cyclohexyl) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hcxoxy).

In some embodiments, at least one R₆₁″ is L-C₃-C₆ carbocyclyl (e.g., cyclopropyl, cyclobutyl, cyclopentyl, or cyclohexyl) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁″ is L-C₃-C₆ carbocyclyl (e.g., cyclopropyl, cyclobutyl, cyclopentyl, or cyclohexyl) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-C₃-C₆ carbocyclyl (e.g., cyclopropyl, cyclobutyl, cyclopentyl, or cyclohexyl) substituted with at least one O—(CH₂)₀₋₃—C₆-C₁₀ aryl (e.g., phenyl or naphthyl).

In some embodiments, at least one R₆₁″ is L-C₃-C₆ carbocyclyl (e.g., cyclopropyl, cyclobutyl, cyclopentyl, or cyclohexyl) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁″ is L-C₃-C₆ carbocyclyl (e.g., cyclopropyl, cyclobutyl, cyclopentyl, or cyclohexyl) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-C₃-C₆ carbocyclyl (e.g., cyclopropyl, cyclobutyl, cyclopentyl, or cyclohexyl) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, 1-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hcxoxy).

In some embodiments, at least one R₆₁″ is L-C₃-C₆ carbocyclyl (e.g., cyclopropyl, cyclobutyl, cyclopentyl, or cyclohexyl) substituted with at least one OH.

In some embodiments, at least one R₆₁″ is L-C₃-C₆ carbocyclyl (e.g., cyclopropyl, cyclobutyl, cyclopentyl, or cyclohexyl) substituted with at least one amino.

In some embodiments, at least one R₆₁″ is L-C₃-C₆ carbocyclyl (e.g., cyclopropyl, cyclobutyl, cyclopentyl, or cyclohexyl) substituted with at least one NO₂.

In some embodiments, at least one R₆₁″ is L-C₃-C₆ carbocyclyl (e.g., cyclopropyl, cyclobutyl, cyclopentyl, or cyclohexyl) substituted with at least one halogen (e.g., F, Cl, Br, or I).

In some embodiments, at least one R₆₁″ is L-C₃-C₆ carbocyclyl (e.g., cyclopropyl, cyclobutyl, cyclopentyl, or cyclohexyl) substituted with at least one CN.

In some embodiments, at least one R₆₁″ is L-C₃-C₆ carbocyclyl (e.g., cyclopropyl, cyclobutyl, cyclopentyl, or cyclohexyl) substituted with at least one —O.

In some embodiments, at least one R₆₁″ is L-C₃-C₆ carbocyclyl (e.g., cyclopropyl, cyclobutyl, cyclopentyl, or cyclohexyl) substituted with at least one N(R)C(O)R₆₂.

In some embodiments, at least one R₆₁″ is L-C₃-C₆ carbocyclyl (e.g., cyclopropyl, cyclobutyl, cyclopentyl, or cyclohexyl) substituted with at least one N(R_{N})C(O)OR₆₂.

In some embodiments, at least one R₆₁″ is L-C₃-C₆ carbocyclyl (e.g., cyclopropyl, cyclobutyl, cyclopentyl, or cyclohexyl) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₃-C₆ carbocyclyl (e.g., cyclopropyl, cyclobutyl, cyclopentyl, or cyclohexyl) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁″ is L-C₃-C₆ carbocyclyl (e.g., cyclopropyl, cyclobutyl, cyclopentyl, or cyclohexyl) substituted with at least one S(O)₁-2NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₃-C₆ carbocyclyl (e.g., cyclopropyl, cyclobutyl, cyclopentyl, or cyclohexyl) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁″ is L-C₃-C₆ carbocyclyl (e.g., cyclopropyl, cyclobutyl, cyclopentyl, or cyclohexyl) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-C₃-C₆ carbocyclyl (e.g., cyclopropyl, cyclobutyl, cyclopentyl, or cyclohexyl) substituted with at least one C(O)R₆₂.

In some embodiments, at least one R₆₁″ is L-C₃-C₆ carbocyclyl (e.g., cyclopropyl, cyclobutyl, cyclopentyl, or cyclohexyl) substituted with at least one C(O)OR₆₂.

In some embodiments, at least one R₆₁″ is L-C₃-C₆ carbocyclyl (e.g., cyclopropyl, cyclobutyl, cyclopentyl, or cyclohexyl) substituted with at least one C(O)NR₆₃R₆₄.

In some embodiments, at least one R₆₁″ is L-C₃-C₆ carbocyclyl (e.g., cyclopropyl, cyclobutyl, cyclopentyl, or cyclohexyl) substituted with at least one P(═O) (OR₆₂)₂.

In some embodiments, at least one R₆₁″ is L-C₃-C₆ carbocyclyl (e.g., cyclopropyl, cyclobutyl, cyclopentyl, or cyclohexyl) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁″ is L-C₃-C₆ carbocyclyl (e.g., cyclopropyl, cyclobutyl, cyclopentyl, or cyclohexyl) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-C₃-C₆ carbocyclyl (e.g., cyclopropyl, cyclobutyl, cyclopentyl, or cyclohexyl) substituted with at least one C₆-C₁₀ aryl (e.g., phenyl or naphthyl).

In some embodiments, at least one R₆₁″ is L-C₃-C₆ carbocyclyl (e.g., cyclopropyl, cyclobutyl, cyclopentyl, or cyclohexyl) substituted with at least one heteroaryl comprising one or two 5- or 6-membered rings and 1 to 4 heteroatoms selected from N, O, and S (e.g., heteroaryl described herein).

In some embodiments, at least one R₆₁″ is L-C₃-C₆ carbocyclyl (e.g., cyclopropyl, cyclobutyl, cyclopentyl, or cyclohexyl), wherein the carbocyclyl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁″ is L-C₃-C₆ carbocyclyl (e.g., cyclopropyl, cyclobutyl, cyclopentyl, or cyclohexyl), wherein the carbocyclyl is substituted and one or more hydrogen atoms in the substituent group may be replaced with one or more deuterium atoms.

In some embodiments, at least one R₆₁″ is L-heterocyclyl comprising one or two 4- to 6-membered rings and 1 to 4 heteroatoms selected from N, O, and S (e.g., heterocyclyl described herein), optionally substituted with one or more groups independently selected from:
C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)),
(CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
O—(CH₂)₀₋₃—C₃-C₆ carbocyclyl (wherein the C₃-C₆ carbocyclyl is cyclopropyl, cyclobutyl, cyclopentyl, or cyclohexyl),
O—(CH₂)₀₋₃-heterocyclyl comprising one or two 3- to 6-membered rings and 1 to 4 heteroatoms selected from N, O, and S (e.g., heterocyclyl described herein),
O—(CH₂)₀₋₃—C₆-C₁₀ aryl (e.g., phenyl or naphthyl),
O—(CH₂)₀₋₃-heteroaryl comprising one or two 5- or 6-membered rings and 1 to 4 heteroatoms selected from N, O, and S (e.g., heteroaryl described herein),
C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy), di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy),
OH, amino, NO₂, halogen (e.g., F, Cl, Br, or I), CN, ═O,
N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl and wherein the C₃-C₆ carbocyclyl is cyclopropyl, cyclobutyl, cyclopentyl, or cyclohexyl,
C(O)R₆₂, C(O)OR₆₂, C(O)NR₆₃R₆₄, P(═O) (OR₆₂)₂, heterocyclyl comprising one or two 3- to 6-membered rings and 1 to 4 heteroatoms selected from N, O, and S (e.g., heterocyclyl described herein), C₆-C₁₀ aryl (e.g., phenyl or naphthyl), and heteroaryl comprising one or two 5- or 6-membered rings and 1 to 4 heteroatoms selected from N, O, and S (e.g., heteroaryl described herein), wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C₁-C₆ alkyl (e.g., methyl, ethyl, propyl, i-propyl, n-butyl, i-butyl, s-butyl, t-butyl, pentyl, or hexyl), C₁-C₆ haloalkyl (e.g., methyl, ethyl, propyl, i-propyl, n-butyl, i-butyl, s-butyl, t-butyl, pentyl, or hexyl, each substituted with one or more halogen (e.g., F, Cl, Br, or I)).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one (CH₂)₀₋₃—C₁-C₆ alkoxy (wherein the alkoxy is methoxy, ethoxy, propoxy, i-propoxy, n-butoxy, i-butoxy, s-butoxy, t-butoxy, pentoxy, or hexoxy) optionally substituted with one or more substituents selected from halogen (e.g., F, Cl, Br, or I), C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one O—(CH₂)₀₋₃—C₃-C₆ carbocyclyl (wherein the C₃-C₆ carbocyclyl is cyclopropyl, cyclobutyl, cyclopentyl, or cyclohexyl and is optionally substituted with one or more R_{A}).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one O—(CH₂)₀₋₃-heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one O—(CH₂)₀₋₃—C₆-C₁₀ aryl (e.g., phenyl or naphthyl).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one O—(CH₂)₀₋₃-heteroaryl comprising one or two 5- or 6-membered rings and 1 to 4 heteroatoms selected from N, O, and S (e.g., heteroaryl described herein).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C₁-C₆ alkyl amino (wherein the alkyl is methyl, ethyl, propyl, i-propyl, n-butyl, i-butyl, s-butyl, t-butyl, pentyl, or hexyl), wherein alkyl is optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one di-C₁-C₆ alkyl amino (wherein each alkyl is independently methyl, ethyl, propyl, i-propyl, n-butyl, i-butyl, s-butyl, t-butyl, pentyl, or hexyl), wherein each alkyl is independently optionally substituted with one or more substituents selected from C₁-C₆ alkyl amino (wherein the alkyl is methyl, ethyl, propyl, i-propyl, n-butyl, i-butyl, s-butyl, t-butyl, pentyl, or hexyl), di-C₁-C₆ alkyl amino (wherein each alkyl is independently methyl, ethyl, propyl, i-propyl, n-butyl, i-butyl, s-butyl, t-butyl, pentyl, or hexyl), and C₁-C₆ alkoxy (wherein the alkoxy is methoxy, ethoxy, propoxy, i-propoxy, n-butoxy, i-butoxy, s-butoxy, t-butoxy, pentoxy, or hexoxy).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one OH.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one amino.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one NO₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one halogen (e.g., F, Cl, Br, or I).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one CN.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one ═O.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one N(R_{N})C(O)R₆₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one N(R_{N})C(O)OR₆₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one N(R_{N})S(O)₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one N(R_{N})S(O)₂—C₃-C₆ cycloalkyl, wherein the C₃-C₆ carbocyclyl is cyclopropyl, cyclobutyl, cyclopentyl, or cyclohexyl and is optionally substituted with one or more R_{A}.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one S(O)₁₋₂NH(C₁-C₆ alkyl), wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one S(O)₁₋₂N(C₁-C₆ alkyl)₂, wherein each C₁-C₆ alkyl is independently methyl, ethyl, propyl, i-propyl, n-butyl, i-butyl, s-butyl, t-butyl, pentyl, or hexyl.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one S(O)₀₋₂—C₁-C₆ alkyl, wherein the C₁-C₆ alkyl is methyl, ethyl, propyl, i-propyl, n-butyl, i-butyl, s-butyl, t-butyl, pentyl, or hexyl.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C(O)R₆₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C(O)OR₆₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C(O)NR₆₃R₆₄.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one P(═O) (OR₆₂)₂.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C₃-C₆ carbocyclyl (e.g., cyclopropyl, cyclobutyl, cyclopentyl, or cyclohexyl), wherein the carbocyclyl is optionally substituted with one or more R_{A}.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one heterocyclyl comprising one or two 3- to 6-membered rings and 1 to 4 heteroatoms selected from N, O, and S (e.g., heterocyclyl described herein), wherein the heterocyclyl is optionally substituted with one or more R_{A}.

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one C₆-C₁₀ aryl (e.g., phenyl or naphthyl).

In some embodiments, at least one R₆₁″ is L-heterocyclyl comprising one or two 4- to 6-membered rings and 1 to 4 heteroatoms selected from N, O, and S (e.g., heterocyclyl described herein) substituted with at least one heteroaryl comprising one or two 5- or 6-membered rings and 1 to 4 heteroatoms selected from N, O, and S (e.g., heteroaryl described herein).

In some embodiments, at least one R₆₁″ is L-heterocyclyl comprising one or two 4- to 6-membered rings and 1 to 4 heteroatoms selected from N, O, and S (e.g., heterocyclyl described herein), wherein the heterocyclyl is substituted and two of the substituent groups, together with the carbon atom(s) to which they are attached, form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}.

In some embodiments, at least one R₆₁″ is L-heterocyclyl comprising one or two 4- to 6-membered rings and 1 to 4 heteroatoms selected from N, O, and S (e.g., heterocyclyl described herein), wherein the heterocyclyl is substituted and one or more hydrogen atoms in the substituent group may be replaced with one or more deuterium atoms.

In some embodiments, a compound of the present application is selected from Table A, or a pharmaceutically acceptable salt, solvate, prodrug, stereoisomer, or tautomer thereof.

In some embodiments, a compound of the present application is selected from Table B, or a pharmaceutically acceptable salt, solvate, prodrug, stereoisomer, or tautomer thereof.

In some embodiments, a compound of the present application is selected from Table C, or a pharmaceutically acceptable salt, solvate, prodrug, stereoisomer, or tautomer thereof.

In another aspect, the present application relates to a pharmaceutical composition comprising a compound described herein or a pharmaceutically acceptable salt, solvate, prodrug, stereoisomer, or tautomer thereof, and a pharmaceutically acceptable diluent, carrier, or excipient.

In another aspect, the present application relates to compounds and compositions thereof (e.g., compounds and compositions described herein, such as the compounds of the formulae or compounds listed in the Tables described here) that are capable of degrading HuR. In some embodiments the, compounds and compositions thereof the present application are capable of selectively degrading HuR, i.e., capable of degrading HuR more effectively, for example, at a lower IC₅₀, as compared to degrading other targets, such as Wee1 and/or GSPT1. In some embodiments, the IC₅₀ of degrading HuR and other targets can be assessed by methods described herein and those commonly used.

In another aspect, the present application relates to a method of treating or preventing a disease or disorder (e.g., a HuR-mediated disease or disorder or a disease or disorder in which HuR plays a role), as described herein. The method comprises administering to a subject in need thereof a therapeutically effective amount of a compound described herein, or a pharmaceutically acceptable salt, solvate, prodrug, stereoisomer, or tautomer thereof, or a therapeutically effective amount of a pharmaceutical composition described herein.

In another aspect, the present application relates to a compound described herein, or a pharmaceutically acceptable salt, solvate, prodrug, stereoisomer, or tautomer thereof, or a pharmaceutical composition described herein, for use in a method of treating or preventing a disease or disorder or of modulating (e.g., deactivating or degrading) HuR.

In another aspect, the present application relates to use of a compound described herein, or a pharmaceutically acceptable salt, solvate, prodrug, stereoisomer, or tautomer thereof, or a pharmaceutical composition described herein, in the manufacture of a medicament for treating or preventing a disease or disorder or for modulating (e.g., deactivating or degrading) HuR.

In some embodiments, the disease or disorder is a cell proliferative disease or disorder. In some embodiments, the cell proliferative disease or disorder cancer. In some embodiments, the cancer is selected from bladder cancer, brain cancer, breast cancer, colorectal cancer, cervical cancer, gastrointestinal cancer, genitourinary cancer, glioma, head and neck cancer, lung cancer, ovarian cancer, pancreatic cancer, prostate cancer, renal cancer, skin cancer, and testicular cancer.

Any of the moieties described herein for any one of the variables in the Formulae herein can be combined with any of the moieties described herein for one or more of the remainder of the variables in the Formulae herein.

Non-limiting illustrative compounds of the application are listed in Tables A-C. As shown in Tables A-C, other tables of compounds, examples, schemes, and compounds throughout the present application, “or 1” (or “Or 1”) and “or 2” (or “Or 2”) indicate a single stereoisomeric configuration although the absolute stereochemistry of the indicated chiral carbon atom is not determined, and “&1” indicates a mixture of the stereoisomers of the indicated chiral carbon atom.

TABLE A
Non-limiting illustrative compounds of the application

| Cmpd | |
|---|---|
| No. | Structure |
| 1. | 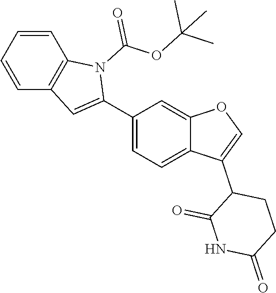 [SMILES: CC(C)(C)OC(=O)n1c(-c2ccc3c(C4CCC(=O)NC4=O)coc3c2)cc2ccccc21] |
| 2. | 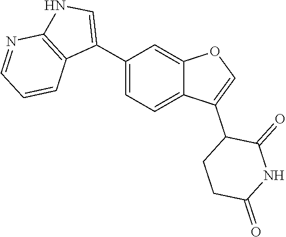 [SMILES: O=C1CCC(c2coc3cc(-c4c[nH]c5ncccc45)ccc23)C(=O)N1] |
| 3. | 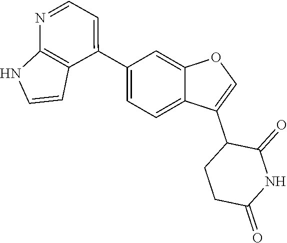 [SMILES: O=C1CCC(c2coc3cc(-c4ccnc5[nH]ccc45)ccc23)C(=O)N1] |
| 4. | 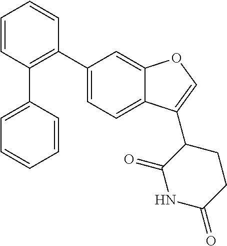 [SMILES: O=C1CCC(c2coc3cc(-c4ccccc4-c4ccccc4)ccc23)C(=O)N1] |
| 5. | 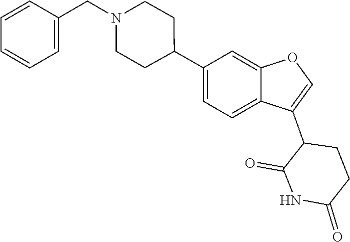 [SMILES: O=C1CCC(c2coc3cc(C4CCN(Cc5ccccc5)CC4)ccc23)C(=O)N1] |
| 6. | 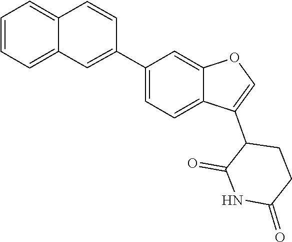 [SMILES: O=C1CCC(c2coc3cc(-c4ccc5ccccc5c4)ccc23)C(=O)N1] |
| 7. | 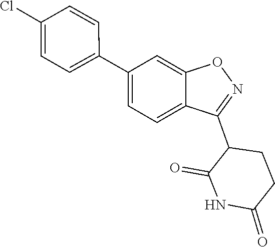 [SMILES: O=C1CCC(c2noc3cc(-c4ccc(Cl)cc4)ccc23)C(=O)N1] |
| 8. | 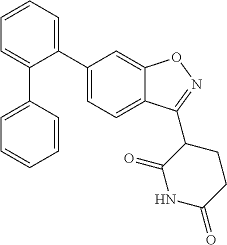 [SMILES: O=C1CCC(c2noc3cc(-c4ccccc4-c4ccccc4)ccc23)C(=O)N1] |
| 9. | 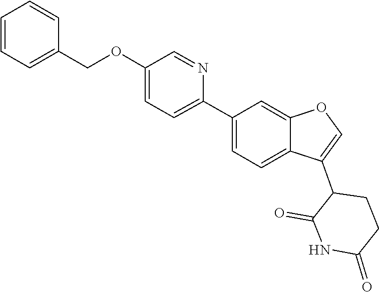 [SMILES: O=C1CCC(c2coc3cc(-c4ccc(OCc5ccccc5)cn4)ccc23)C(=O)N1] |
| 10. | 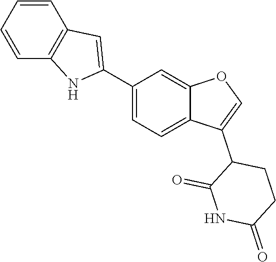 [SMILES: O=C1CCC(c2coc3cc(-c4cc5ccccc5[nH]4)ccc23)C(=O)N1] |
| 11. | 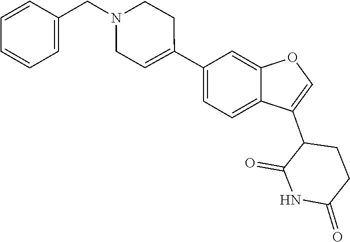 [SMILES: O=C1CCC(c2coc3cc(C4=CCN(Cc5ccccc5)CC4)ccc23)C(=O)N1] |
| 12. | 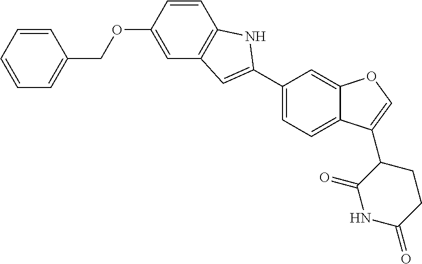 [SMILES: O=C1CCC(c2coc3cc(-c4cc5cc(OCc6ccccc6)ccc5[nH]4)ccc23)C(=O)N1] |
| 13. | 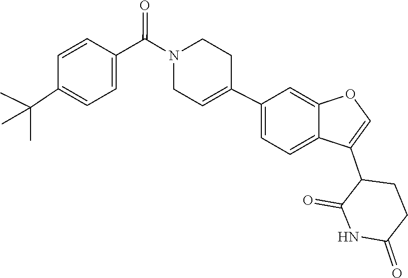 [SMILES: CC(C)(C)c1ccc(C(=O)N2CC=C(c3ccc4c(C5CCC(=O)NC5=O)coc4c3)CC2)cc1] |
| 14. | 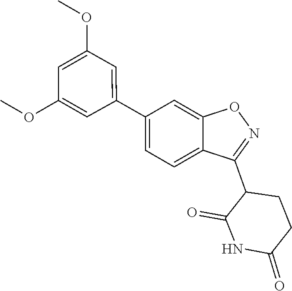 [SMILES: COc1cc(OC)cc(-c2ccc3c(C4CCC(=O)NC4=O)noc3c2)c1] |
| 15. | 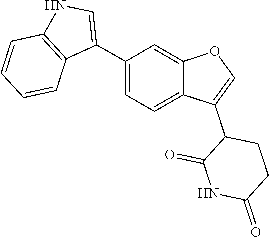 [SMILES: O=C1CCC(c2coc3cc(-c4c[nH]c5ccccc45)ccc23)C(=O)N1] |
| 16. | 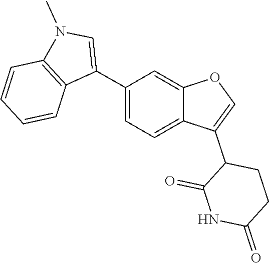 [SMILES: Cn1cc(-c2ccc3c(C4CCC(=O)NC4=O)coc3c2)c2ccccc21] |
| 17. | 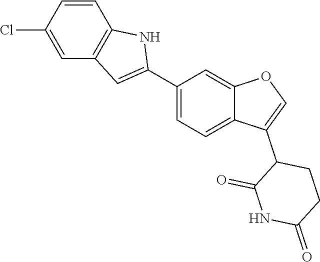 [SMILES: O=C1CCC(c2coc3cc(-c4cc5cc(Cl)ccc5[nH]4)ccc23)C(=O)N1] |
| 18. | 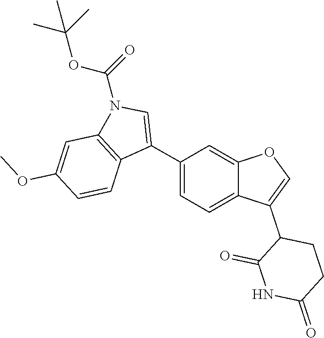 [SMILES: COc1ccc2c(-c3ccc4c(C5CCC(=O)NC5=O)coc4c3)cn(C(=O)OC(C)(C)C)c2c1] |
| 19. | 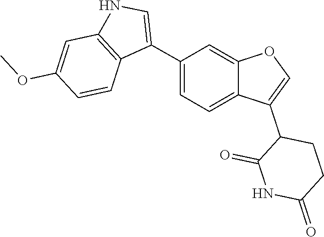 [SMILES: COc1ccc2c(-c3ccc4c(C5CCC(=O)NC5=O)coc4c3)c[nH]c2c1] |
| 20. | 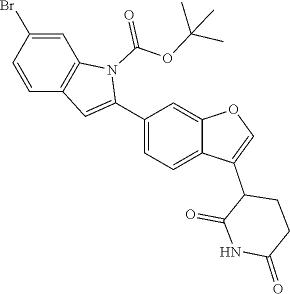 [SMILES: CC(C)(C)OC(=O)n1c(-c2ccc3c(C4CCC(=O)NC4=O)coc3c2)cc2ccc(Br)cc21] |
| 21. | 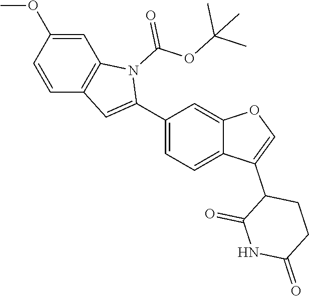 [SMILES: COc1ccc2cc(-c3ccc4c(C5CCC(=O)NC5=O)coc4c3)n(C(=O)OC(C)(C)C)c2c1] |
| 22. | 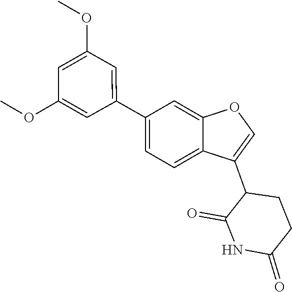 [SMILES: COc1cc(OC)cc(-c2ccc3c(C4CCC(=O)NC4=O)coc3c2)c1] |
| 23. | 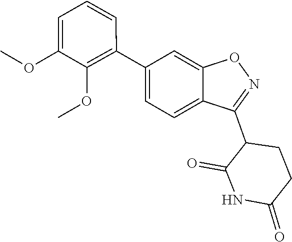 [SMILES: COc1cccc(-c2ccc3c(C4CCC(=O)NC4=O)noc3c2)c1OC] |
| 24. | 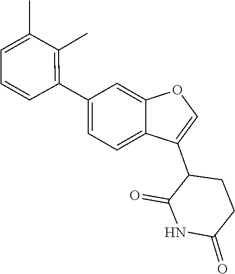 [SMILES: Cc1cccc(-c2ccc3c(C4CCC(=O)NC4=O)coc3c2)c1C] |
| 25. | 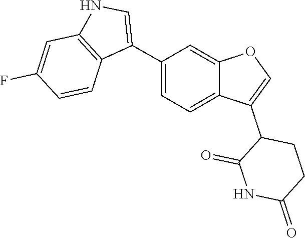 [SMILES: O=C1CCC(c2coc3cc(-c4c[nH]c5cc(F)ccc45)ccc23)C(=O)N1] |
| 26. | 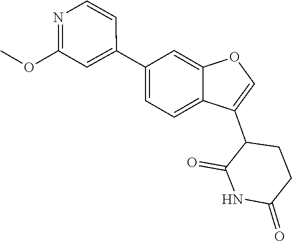 [SMILES: COc1cc(-c2ccc3c(C4CCC(=O)NC4=O)coc3c2)ccn1] |
| 27. | 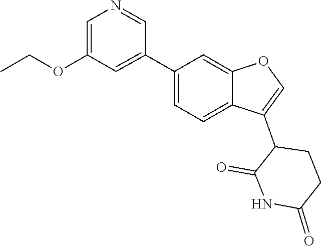 [SMILES: CCOc1cncc(-c2ccc3c(C4CCC(=O)NC4=O)coc3c2)c1] |
| 28. | 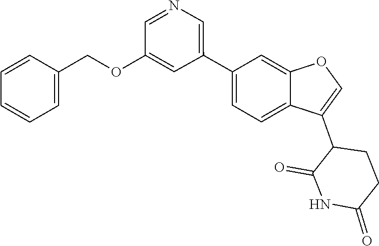 [SMILES: O=C1CCC(c2coc3cc(-c4cncc(OCc5ccccc5)c4)ccc23)C(=O)N1] |
| 29. | 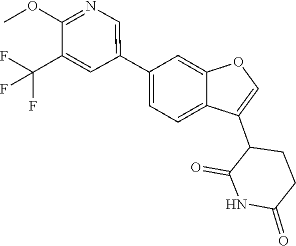 [SMILES: COc1ncc(-c2ccc3c(C4CCC(=O)NC4=O)coc3c2)cc1C(F)(F)F] |
| 30. | 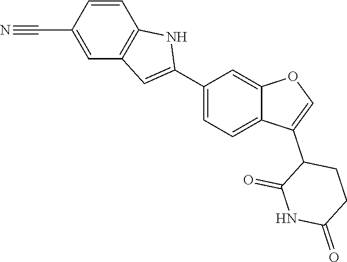 [SMILES: N#Cc1ccc2[nH]c(-c3ccc4c(C5CCC(=O)NC5=O)coc4c3)cc2c1] |
| 31. | 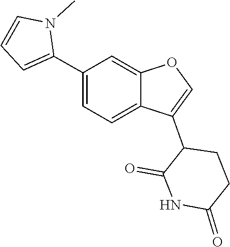 [SMILES: Cn1cccc1-c1ccc2c(C3CCC(=O)NC3=O)coc2c1] |
| 32. | 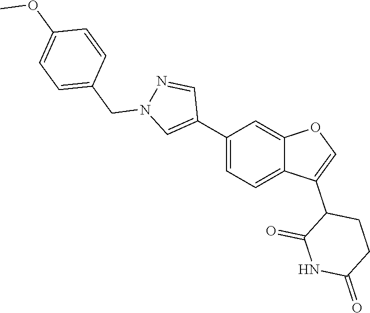 [SMILES: COc1ccc(Cn2cc(-c3ccc4c(C5CCC(=O)NC5=O)coc4c3)cn2)cc1] |
| 33. | 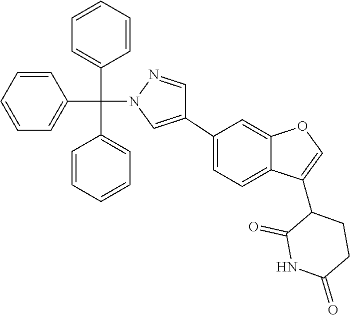 [SMILES: O=C1CCC(c2coc3cc(-c4cnn(C(c5ccccc5)(c5ccccc5)c5ccccc5)c4)ccc23)C(=O)N1] |
| 34. | 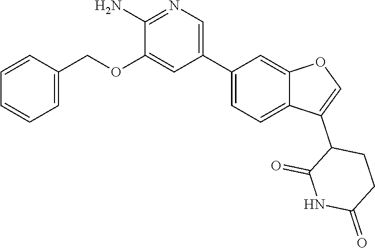 [SMILES: Nc1ncc(-c2ccc3c(C4CCC(=O)NC4=O)coc3c2)cc1OCc1ccccc1] |
| 35. | 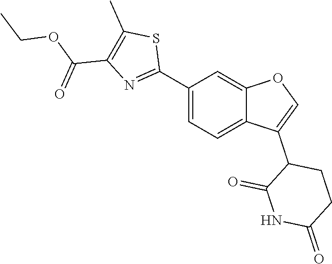 [SMILES: CCOC(=O)c1nc(-c2ccc3c(C4CCC(=O)NC4=O)coc3c2)sc1C] |
| 36. | 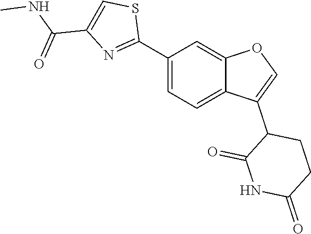 [SMILES: CNC(=O)c1csc(-c2ccc3c(C4CCC(=O)NC4=O)coc3c2)n1] |
| 37. | 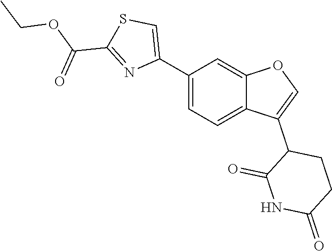 [SMILES: CCOC(=O)c1nc(-c2ccc3c(C4CCC(=O)NC4=O)coc3c2)cs1] |
| 38. | 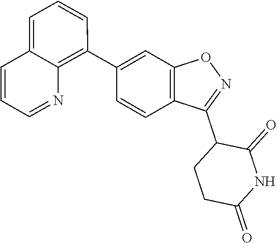 [SMILES: O=C1CCC(c2noc3cc(-c4cccc5cccnc45)ccc23)C(=O)N1] |
| 39. | 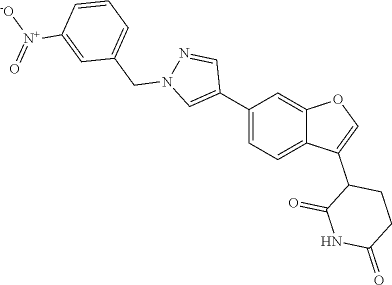 |
| 40. | 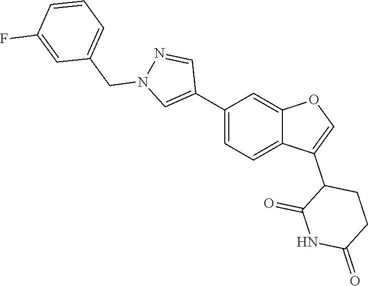 [SMILES: O=C1CCC(c2coc3cc(-c4cnn(Cc5cccc(F)c5)c4)ccc23)C(=O)N1] |
| 41. | 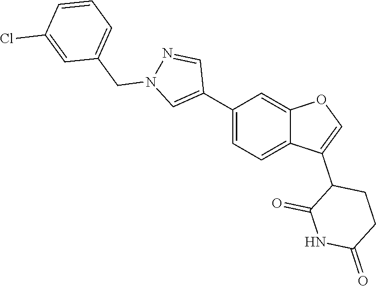 [SMILES: O=C1CCC(c2coc3cc(-c4cnn(Cc5cccc(Cl)c5)c4)ccc23)C(=O)N1] |
| 42. | 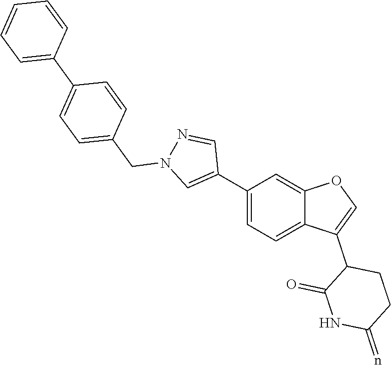 |
| 43. | 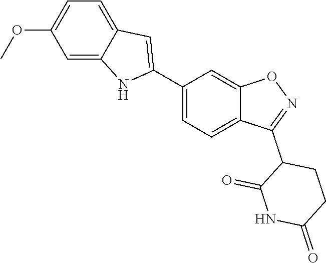 [SMILES: COc1ccc2cc(-c3ccc4c(C5CCC(=O)NC5=O)noc4c3)[nH]c2c1] |
| 44. | 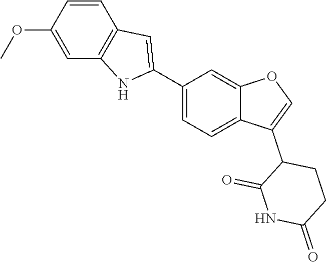 [SMILES: COc1ccc2cc(-c3ccc4c(C5CCC(=O)NC5=O)coc4c3)[nH]c2c1] |
| 45. | 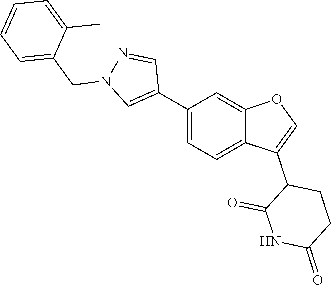 [SMILES: Cc1ccccc1Cn1cc(-c2ccc3c(C4CCC(=O)NC4=O)coc3c2)cn1] |
| 46. | 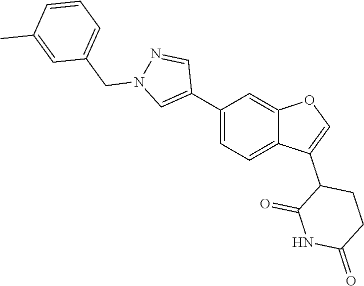 [SMILES: Cc1cccc(Cn2cc(-c3ccc4c(C5CCC(=O)NC5=O)coc4c3)cn2)c1] |
| 47. | 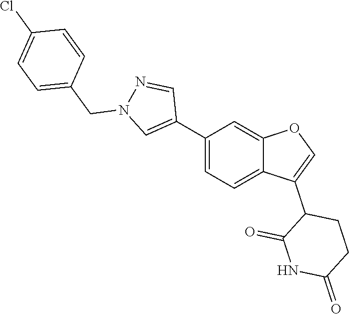 [SMILES: O=C1CCC(c2coc3cc(-c4cnn(Cc5ccc(Cl)cc5)c4)ccc23)C(=O)N1] |
| 48. | 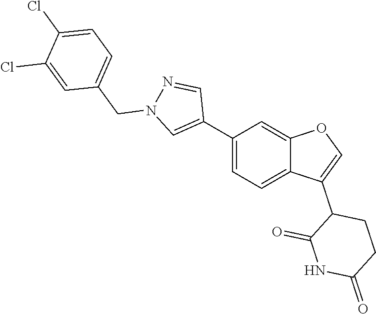 [SMILES: O=C1CCC(c2coc3cc(-c4cnn(Cc5ccc(Cl)c(Cl)c5)c4)ccc23)C(=O)N1] |
| 49. | 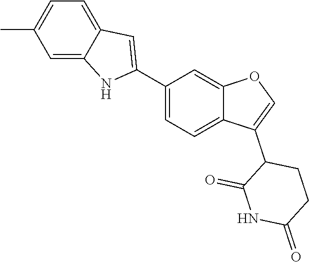 [SMILES: Cc1ccc2cc(-c3ccc4c(C5CCC(=O)NC5=O)coc4c3)[nH]c2c1] |
| 50. | 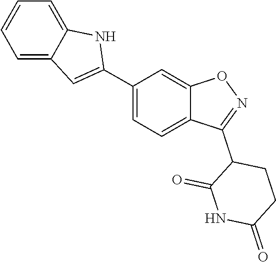 [SMILES: O=C1CCC(c2noc3cc(-c4cc5ccccc5[nH]4)ccc23)C(=O)N1] |
| 51. | 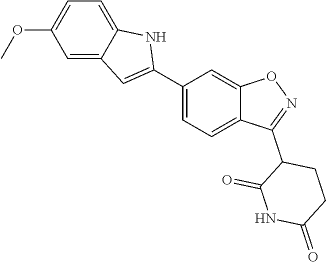 [SMILES: COc1ccc2[nH]c(-c3ccc4c(C5CCC(=O)NC5=O)noc4c3)cc2c1] |
| 52. | 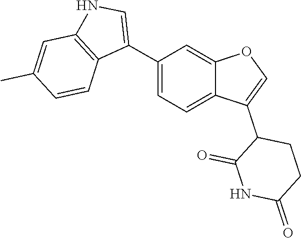 [SMILES: Cc1ccc2c(-c3ccc4c(C5CCC(=O)NC5=O)coc4c3)c[nH]c2c1] |
| 53. | 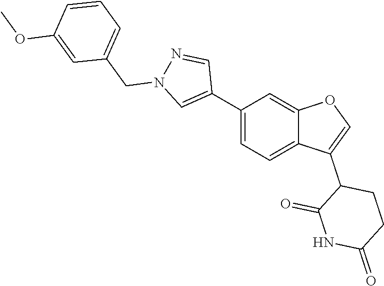 [SMILES: COc1cccc(Cn2cc(-c3ccc4c(C5CCC(=O)NC5=O)coc4c3)cn2)c1] |
| 54. | 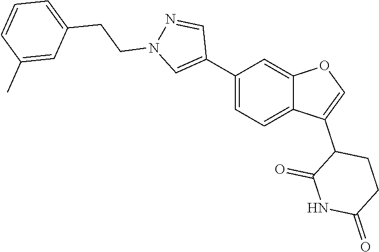 [SMILES: Cc1cccc(CCn2cc(-c3ccc4c(C5CCC(=O)NC5=O)coc4c3)cn2)c1] |
| 55. | 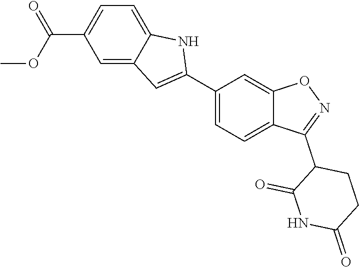 [SMILES: COC(=O)c1ccc2[nH]c(-c3ccc4c(C5CCC(=O)NC5=O)noc4c3)cc2c1] |
| 56. | 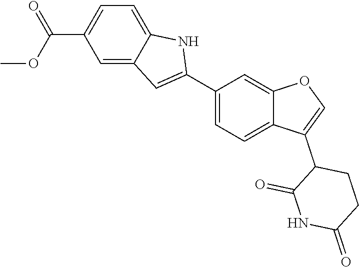 [SMILES: COC(=O)c1ccc2[nH]c(-c3ccc4c(C5CCC(=O)NC5=O)coc4c3)cc2c1] |
| 57. | 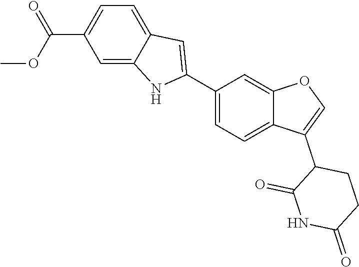 [SMILES: COC(=O)c1ccc2cc(-c3ccc4c(C5CCC(=O)NC5=O)coc4c3)[nH]c2c1] |
| 58. | 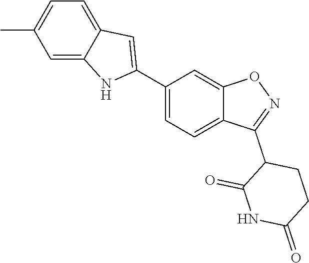 [SMILES: Cc1ccc2cc(-c3ccc4c(C5CCC(=O)NC5=O)noc4c3)[nH]c2c1] |
| 59. | 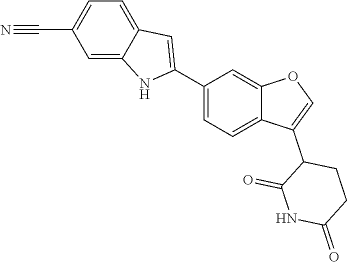 [SMILES: N#Cc1ccc2cc(-c3ccc4c(C5CCC(=O)NC5=O)coc4c3)[nH]c2c1] |
| 60. | 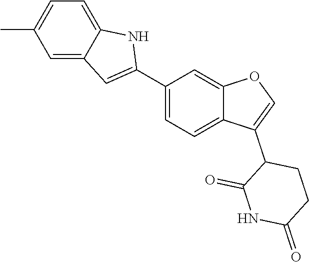 [SMILES: Cc1ccc2[nH]c(-c3ccc4c(C5CCC(=O)NC5=O)coc4c3)cc2c1] |
| 61. | 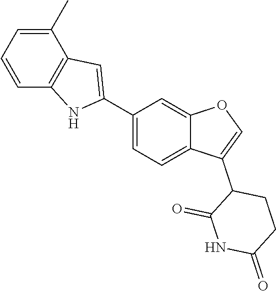 [SMILES: Cc1cccc2[nH]c(-c3ccc4c(C5CCC(=O)NC5=O)coc4c3)cc12] |
| 62. | 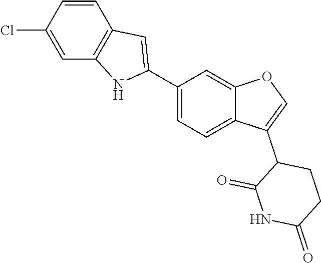 [SMILES: O=C1CCC(c2coc3cc(-c4cc5ccc(Cl)cc5[nH]4)ccc23)C(=O)N1] |
| 63. | 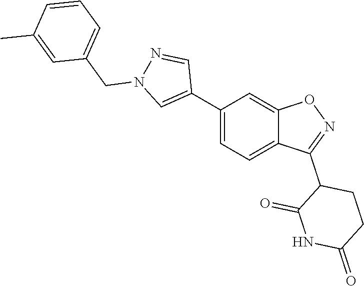 [SMILES: Cc1cccc(Cn2cc(-c3ccc4c(C5CCC(=O)NC5=O)noc4c3)cn2)c1] |
| 64. | 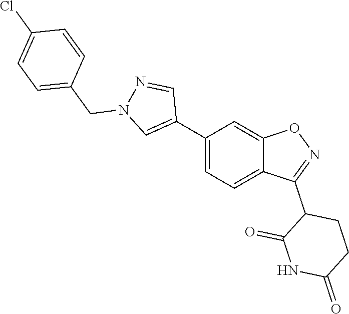 [SMILES: O=C1CCC(c2noc3cc(-c4cnn(Cc5ccc(Cl)cc5)c4)ccc23)C(=O)N1] |
| 65. | 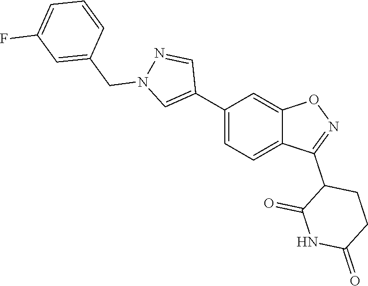 [SMILES: O=C1CCC(c2noc3cc(-c4cnn(Cc5cccc(F)c5)c4)ccc23)C(=O)N1] |
| 66. | 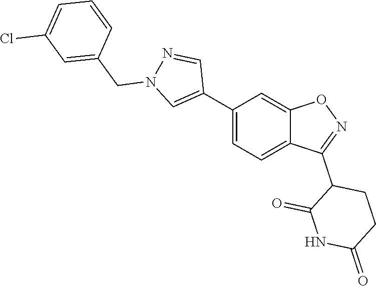 [SMILES: O=C1CCC(c2noc3cc(-c4cnn(Cc5cccc(Cl)c5)c4)ccc23)C(=O)N1] |
| 67. | 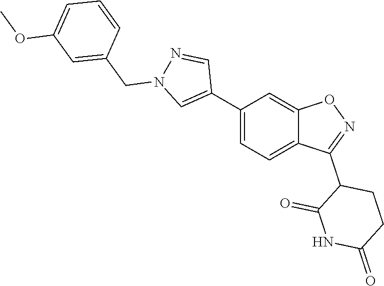 [SMILES: COc1cccc(Cn2cc(-c3ccc4c(C5CCC(=O)NC5=O)noc4c3)cn2)c1] |
| 68. | 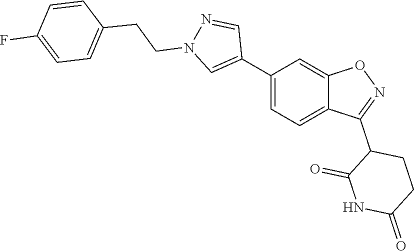 [SMILES: O=C1CCC(c2noc3cc(-c4cnn(CCc5ccc(F)cc5)c4)ccc23)C(=O)N1] |
| 69. | 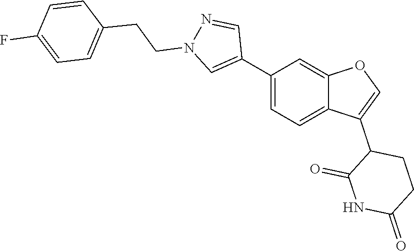 [SMILES: O=C1CCC(c2coc3cc(-c4cnn(CCc5ccc(F)cc5)c4)ccc23)C(=O)N1] |
| 70. | 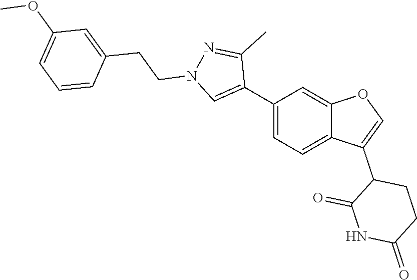 [SMILES: COc1cccc(CCn2cc(-c3ccc4c(C5CCC(=O)NC5=O)coc4c3)c(C)n2)c1] |
| 71. | 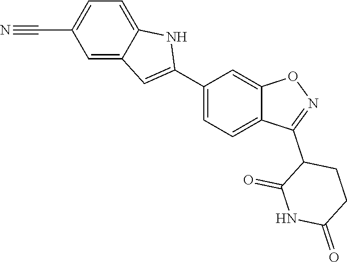 [SMILES: N#Cc1ccc2[nH]c(-c3ccc4c(C5CCC(=O)NC5=O)noc4c3)cc2c1] |
| 72. | 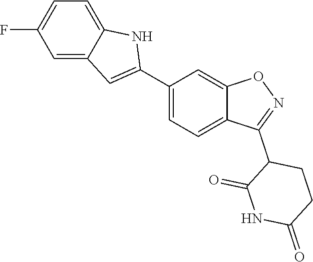 [SMILES: O=C1CCC(c2noc3cc(-c4cc5cc(F)ccc5[nH]4)ccc23)C(=O)N1] |
| 73. | 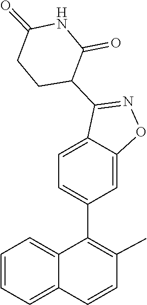 [SMILES: Cc1ccc2ccccc2c1-c1ccc2c(C3CCC(=O)NC3=O)noc2c1] |
| 74. | 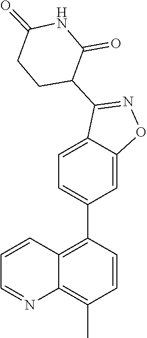 [SMILES: Cc1ccc(-c2ccc3c(C4CCC(=O)NC4=O)noc3c2)c2cccnc12] |
| 75. | 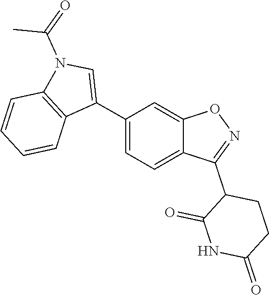 [SMILES: CC(=O)n1cc(-c2ccc3c(C4CCC(=O)NC4=O)noc3c2)c2ccccc21] |
| 76. | 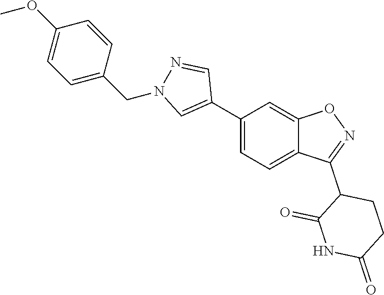 [SMILES: COc1ccc(Cn2cc(-c3ccc4c(C5CCC(=O)NC5=O)noc4c3)cn2)cc1] |
| 77. | 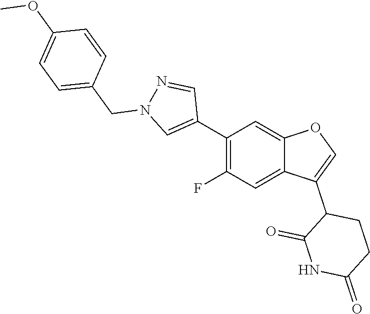 [SMILES: COc1ccc(Cn2cc(-c3cc4occ(C5CCC(=O)NC5=O)c4cc3F)cn2)cc1] |
| 78. | 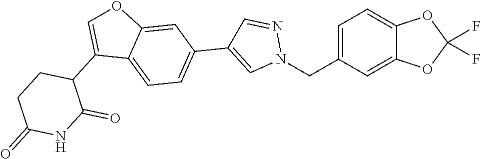 [SMILES: O=C1CCC(c2coc3cc(-c4cnn(Cc5ccc6c(c5)OC(F)(F)O6)c4)ccc23)C(=O)N1] |
| 79. | 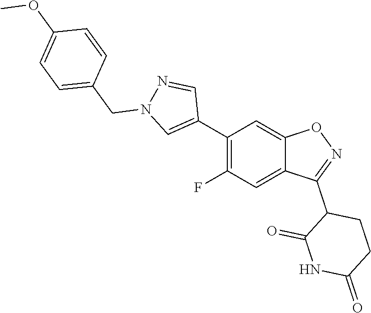 [SMILES: COc1ccc(Cn2cc(-c3cc4onc(C5CCC(=O)NC5=O)c4cc3F)cn2)cc1] |
| 80. | 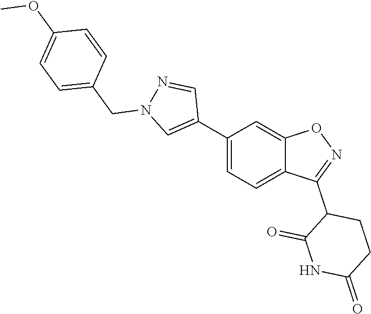 [SMILES: COc1ccc(Cn2cc(-c3ccc4c(C5CCC(=O)NC5=O)noc4c3)cn2)cc1] |
| 81. | 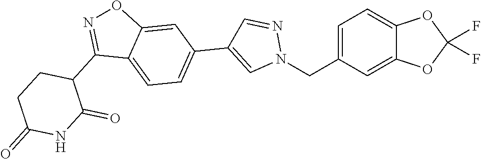 [SMILES: O=C1CCC(c2noc3cc(-c4cnn(Cc5ccc6c(c5)OC(F)(F)O6)c4)ccc23)C(=O)N1] |
| 82. | 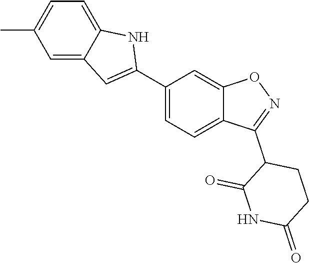 [SMILES: Cc1ccc2[nH]c(-c3ccc4c(C5CCC(=O)NC5=O)noc4c3)cc2c1] |
| 83. | 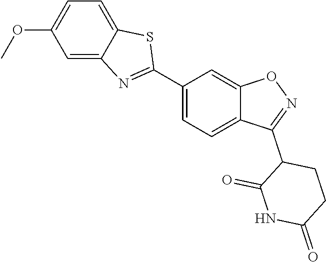 [SMILES: COc1ccc2sc(-c3ccc4c(C5CCC(=O)NC5=O)noc4c3)nc2c1] |
| 84. | 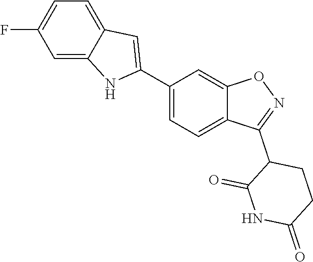 [SMILES: O=C1CCC(c2noc3cc(-c4cc5ccc(F)cc5[nH]4)ccc23)C(=O)N1] |
| 85. | 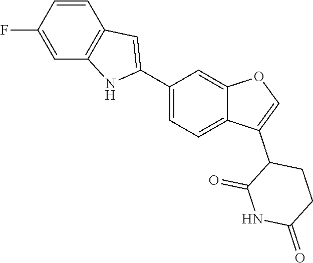 [SMILES: O=C1CCC(c2coc3cc(-c4cc5ccc(F)cc5[nH]4)ccc23)C(=O)N1] |
| 86. | 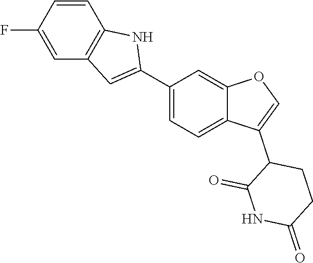 [SMILES: O=C1CCC(c2coc3cc(-c4cc5cc(F)ccc5[nH]4)ccc23)C(=O)N1] |
| 87. | 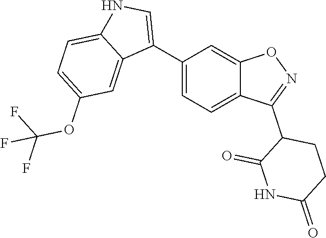 [SMILES: O=C1CCC(c2noc3cc(-c4c[nH]c5ccc(OC(F)(F)F)cc45)ccc23)C(=O)N1] |
| 88. | 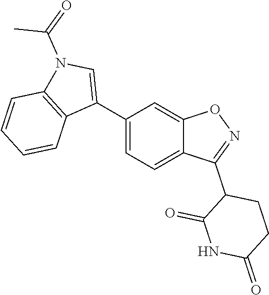 [SMILES: CC(=O)n1cc(-c2ccc3c(C4CCC(=O)NC4=O)noc3c2)c2ccccc21] |
| 89. | 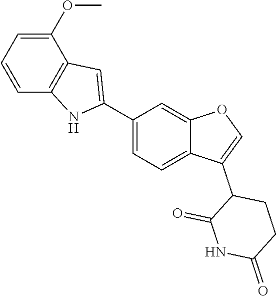 [SMILES: COc1cccc2[nH]c(-c3ccc4c(C5CCC(=O)NC5=O)coc4c3)cc12] |
| 90. | 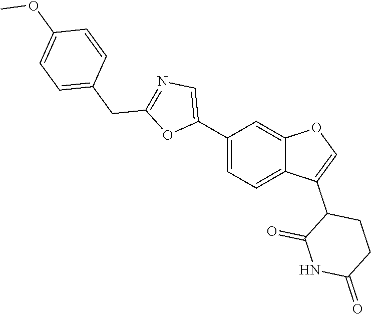 [SMILES: COc1ccc(Cc2ncc(-c3ccc4c(C5CCC(=O)NC5=O)coc4c3)o2)cc1] |
| 91. | 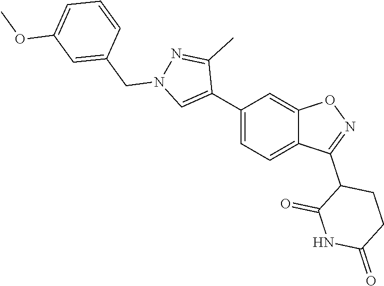 [SMILES: COc1cccc(Cn2cc(-c3ccc4c(C5CCC(=O)NC5=O)noc4c3)c(C)n2)c1] |
| 92. | 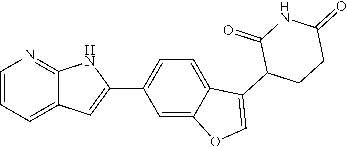 [SMILES: O=C1CCC(c2coc3cc(-c4cc5cccnc5[nH]4)ccc23)C(=O)N1] |
| 93. | 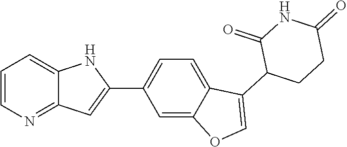 [SMILES: O=C1CCC(c2coc3cc(-c4cc5ncccc5[nH]4)ccc23)C(=O)N1] |
| 94. | 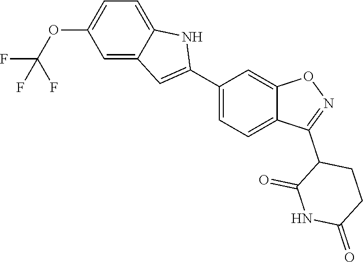 [SMILES: O=C1CCC(c2noc3cc(-c4cc5cc(OC(F)(F)F)ccc5[nH]4)ccc23)C(=O)N1] |
| 95. | 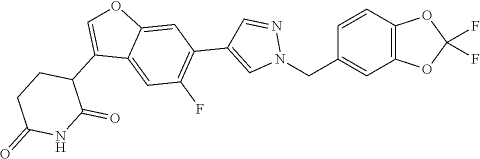 [SMILES: O=C1CCC(c2coc3cc(-c4cnn(Cc5ccc6c(c5)OC(F)(F)O6)c4)c(F)cc23)C(=O)N1] |
| 96. | 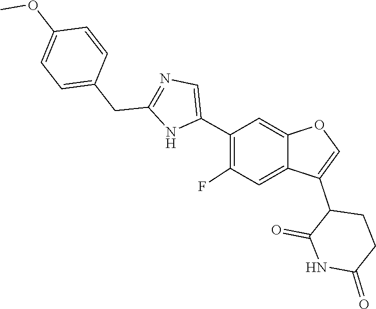 [SMILES: COc1ccc(Cc2ncc(-c3cc4occ(C5CCC(=O)NC5=O)c4cc3F)[nH]2)cc1] |
| 97. | 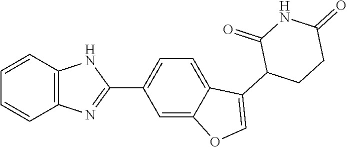 [SMILES: O=C1CCC(c2coc3cc(-c4nc5ccccc5[nH]4)ccc23)C(=O)N1] |
| 98. | 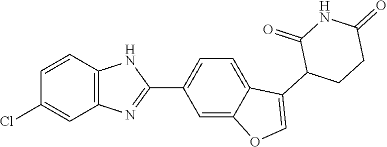 [SMILES: O=C1CCC(c2coc3cc(-c4nc5cc(Cl)ccc5[nH]4)ccc23)C(=O)N1] |
| 99. | 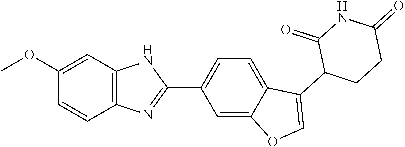 [SMILES: COc1ccc2nc(-c3ccc4c(C5CCC(=O)NC5=O)coc4c3)[nH]c2c1] |
| 100. | 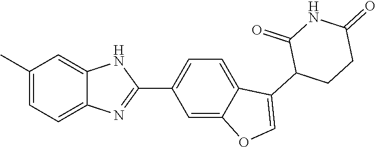 [SMILES: Cc1ccc2nc(-c3ccc4c(C5CCC(=O)NC5=O)coc4c3)[nH]c2c1] |
| 101. | 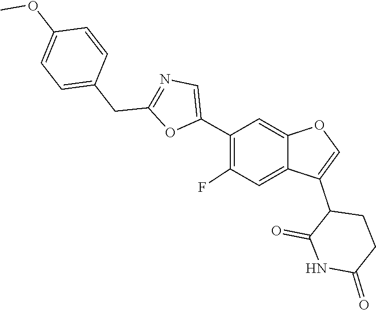 [SMILES: COc1ccc(Cc2ncc(-c3cc4occ(C5CCC(=O)NC5=O)c4cc3F)o2)cc1] |
| 102. | 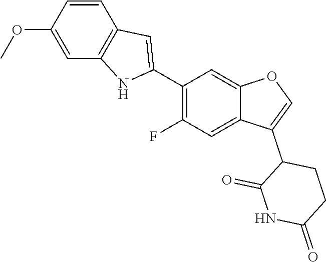 [SMILES: COc1ccc2cc(-c3cc4occ(C5CCC(=O)NC5=O)c4cc3F)[nH]c2c1] |
| 103. | 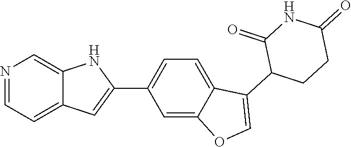 [SMILES: O=C1CCC(c2coc3cc(-c4cc5ccncc5[nH]4)ccc23)C(=O)N1] |
| 104. | 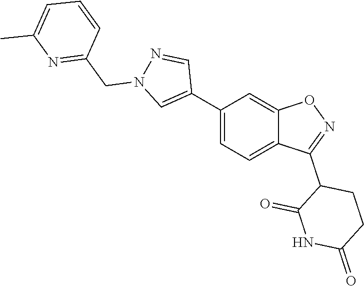 [SMILES: Cc1cccc(Cn2cc(-c3ccc4c(C5CCC(=O)NC5=O)noc4c3)cn2)n1] |
| 105. | 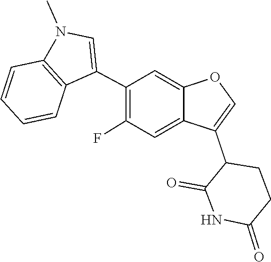 [SMILES: Cn1cc(-c2cc3occ(C4CCC(=O)NC4=O)c3cc2F)c2ccccc21] |
| 106. | 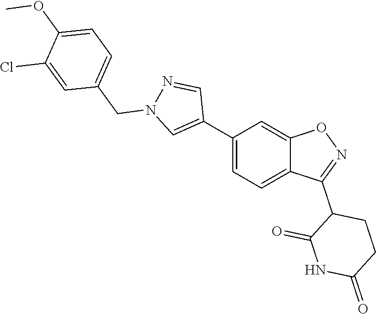 [SMILES: COc1ccc(Cn2cc(-c3ccc4c(C5CCC(=O)NC5=O)noc4c3)cn2)cc1Cl] |
| 107. | 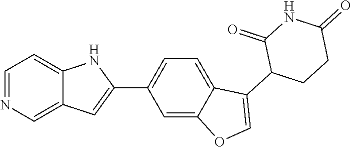 [SMILES: O=C1CCC(c2coc3cc(-c4cc5cnccc5[nH]4)ccc23)C(=O)N1] |
| 108. | 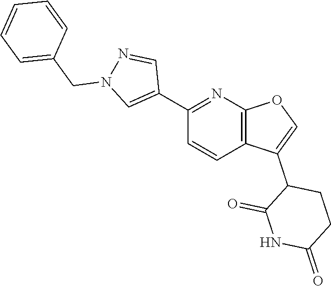 [SMILES: O=C1CCC(c2coc3nc(-c4cnn(Cc5ccccc5)c4)ccc23)C(=O)N1] |
| 109. | 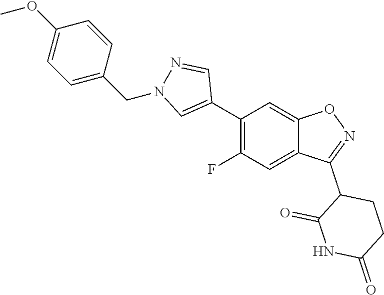 [SMILES: COc1ccc(Cn2cc(-c3cc4onc(C5CCC(=O)NC5=O)c4cc3F)cn2)cc1] |
| 110. | 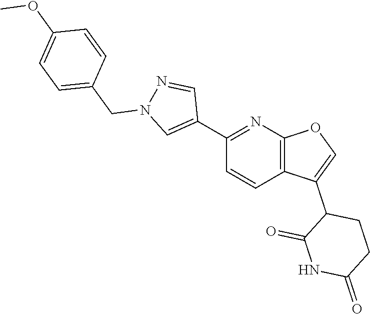 [SMILES: COc1ccc(Cn2cc(-c3ccc4c(C5CCC(=O)NC5=O)coc4n3)cn2)cc1] |
| 111. | 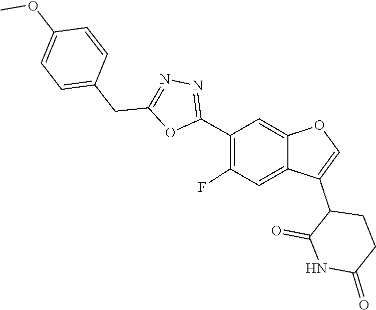 [SMILES: COc1ccc(Cc2nnc(-c3cc4occ(C5CCC(=O)NC5=O)c4cc3F)o2)cc1] |
| 112. | 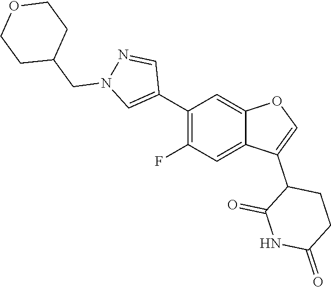 [SMILES: O=C1CCC(c2coc3cc(-c4cnn(CC5CCOCC5)c4)c(F)cc23)C(=O)N1] |
| 113. | 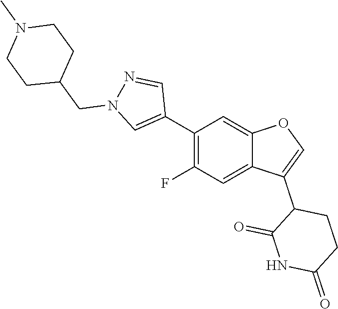 [SMILES: CN1CCC(Cn2cc(-c3cc4occ(C5CCC(=O)NC5=O)c4cc3F)cn2)CC1] |
| 114. | 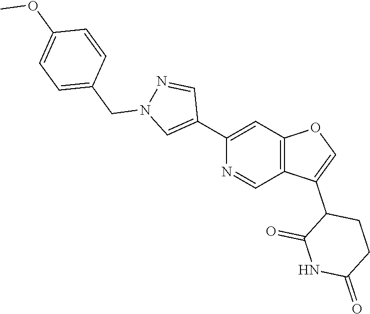 [SMILES: COc1ccc(Cn2cc(-c3cc4occ(C5CCC(=O)NC5=O)c4cn3)cn2)cc1] |
| 115. | 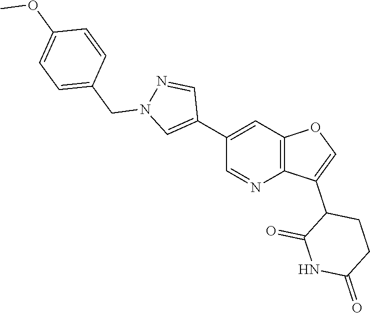 [SMILES: COc1ccc(Cn2cc(-c3cnc4c(C5CCC(=O)NC5=O)coc4c3)cn2)cc1] |
| 116. | 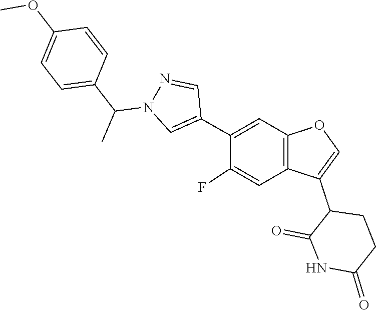 [SMILES: COc1ccc(C(C)n2cc(-c3cc4occ(C5CCC(=O)NC5=O)c4cc3F)cn2)cc1] |
| 117. | 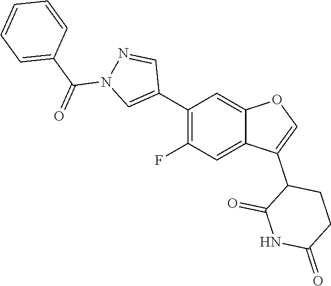 [SMILES: O=C1CCC(c2coc3cc(-c4cnn(C(=O)c5ccccc5)c4)c(F)cc23)C(=O)N1] |
| 118. | 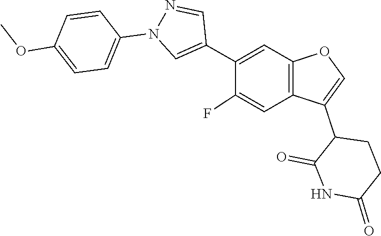 [SMILES: COc1ccc(-n2cc(-c3cc4occ(C5CCC(=O)NC5=O)c4cc3F)cn2)cc1] |
| 119. | 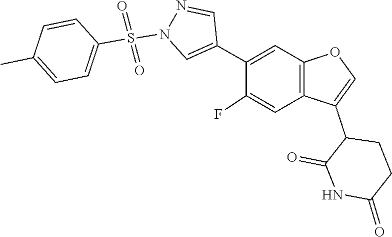 [SMILES: Cc1ccc(S(=O)(=O)n2cc(-c3cc4occ(C5CCC(=O)NC5=O)c4cc3F)cn2)cc1] |
| 120. | 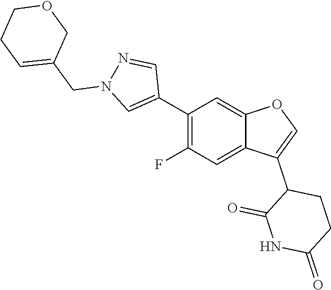 [SMILES: O=C1CCC(c2coc3cc(-c4cnn(CC5=CCCOC5)c4)c(F)cc23)C(=O)N1] |
| 121. | 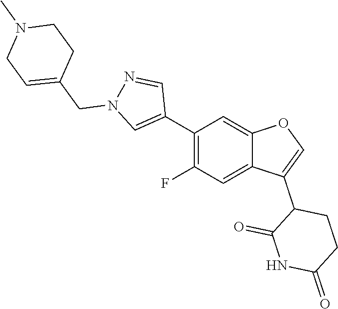 [SMILES: CN1CC=C(Cn2cc(-c3cc4occ(C5CCC(=O)NC5=O)c4cc3F)cn2)CC1] |
| 122. | 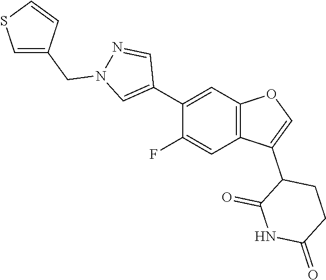 [SMILES: O=C1CCC(c2coc3cc(-c4cnn(Cc5ccsc5)c4)c(F)cc23)C(=O)N1] |
| 123. | 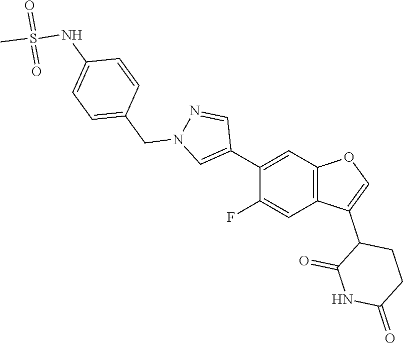 [SMILES: CS(=O)(=O)Nc1ccc(Cn2cc(-c3cc4occ(C5CCC(=O)NC5=O)c4cc3F)cn2)cc1] |
| 124. | 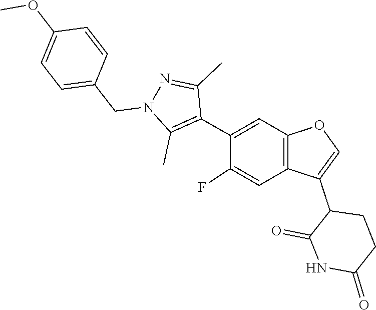 [SMILES: COc1ccc(Cn2nc(C)c(-c3cc4occ(C5CCC(=O)NC5=O)c4cc3F)c2C)cc1] |
| 125. | 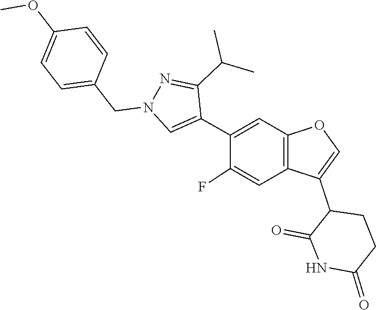 [SMILES: COc1ccc(Cn2cc(-c3cc4occ(C5CCC(=O)NC5=O)c4cc3F)c(C(C)C)n2)cc1] |
| 126. | 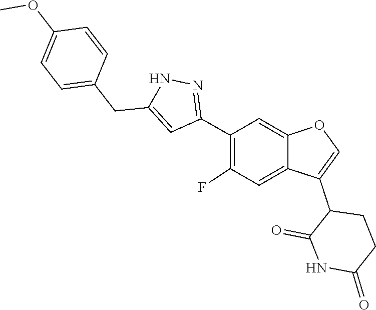 [SMILES: COc1ccc(Cc2cc(-c3cc4occ(C5CCC(=O)NC5=O)c4cc3F)n[nH]2)cc1] |
| 127. | 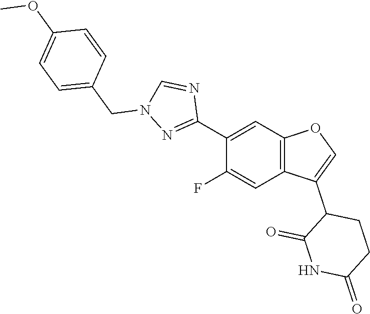 [SMILES: COc1ccc(Cn2cnc(-c3cc4occ(C5CCC(=O)NC5=O)c4cc3F)n2)cc1] |
| 128. | 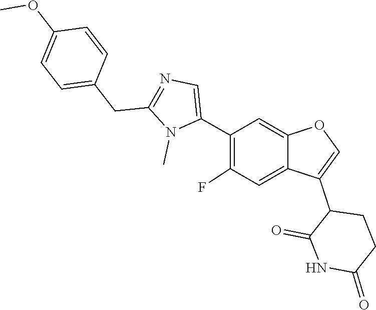 [SMILES: COc1ccc(Cc2ncc(-c3cc4occ(C5CCC(=O)NC5=O)c4cc3F)n2C)cc1] |
| 129. | 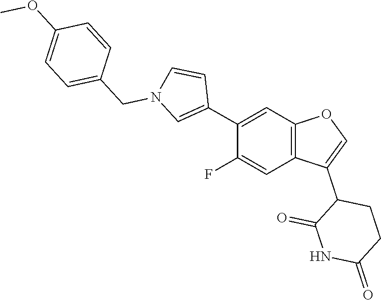 [SMILES: COc1ccc(Cn2ccc(-c3cc4occ(C5CCC(=O)NC5=O)c4cc3F)c2)cc1] |
| 130. | 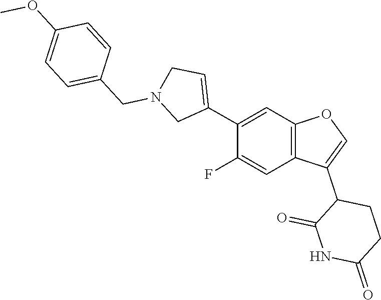 [SMILES: COc1ccc(CN2CC=C(c3cc4occ(C5CCC(=O)NC5=O)c4cc3F)C2)cc1] |
| 131. | 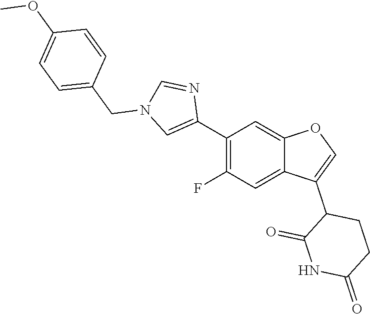 [SMILES: COc1ccc(Cn2cnc(-c3cc4occ(C5CCC(=O)NC5=O)c4cc3F)c2)cc1] |
| 132. | 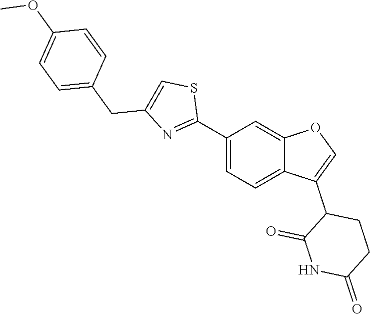 [SMILES: COc1ccc(Cc2csc(-c3ccc4c(C5CCC(=O)NC5=O)coc4c3)n2)cc1] |
| 133. | 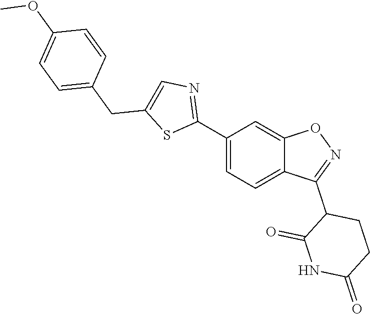 [SMILES: COc1ccc(Cc2cnc(-c3ccc4c(C5CCC(=O)NC5=O)noc4c3)s2)cc1] |
| 134. | 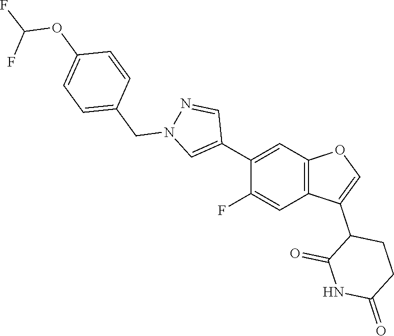 [SMILES: O=C1CCC(c2coc3cc(-c4cnn(Cc5ccc(OC(F)F)cc5)c4)c(F)cc23)C(=O)N1] |
| 135. | 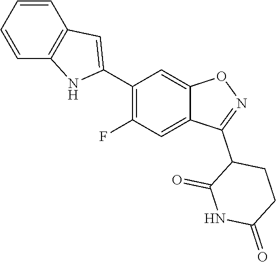 [SMILES: O=C1CCC(c2noc3cc(-c4cc5ccccc5[nH]4)c(F)cc23)C(=O)N1] |
| 136. | 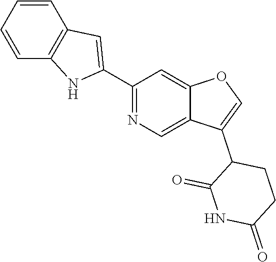 [SMILES: O=C1CCC(c2coc3cc(-c4cc5ccccc5[nH]4)ncc23)C(=O)N1] |
| 137. | 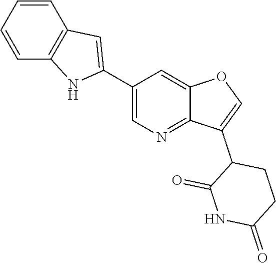 [SMILES: O=C1CCC(c2coc3cc(-c4cc5ccccc5[nH]4)cnc23)C(=O)N1] |
| 138. | 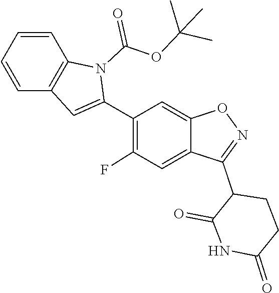 [SMILES: CC(C)(C)OC(=O)n1c(-c2cc3onc(C4CCC(=O)NC4=O)c3cc2F)cc2ccccc21] |
| 139. | 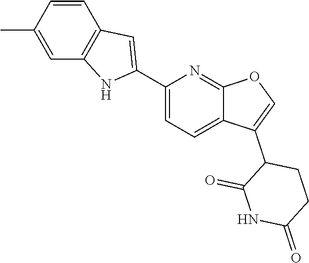 [SMILES: Cc1ccc2cc(-c3ccc4c(C5CCC(=O)NC5=O)coc4n3)[nH]c2c1] |
| 140. | 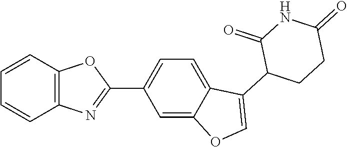 [SMILES: O=C1CCC(c2coc3cc(-c4nc5ccccc5o4)ccc23)C(=O)N1] |
| 141. | 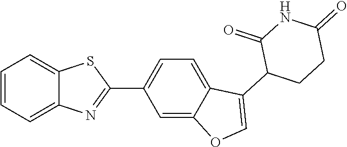 [SMILES: O=C1CCC(c2coc3cc(-c4nc5ccccc5s4)ccc23)C(=O)N1] |
| 142. | 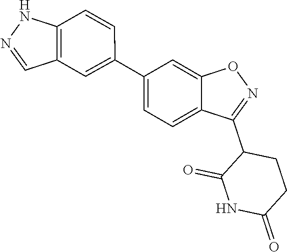 [SMILES: O=C1CCC(c2noc3cc(-c4ccc5[nH]ncc5c4)ccc23)C(=O)N1] |
| 143. | 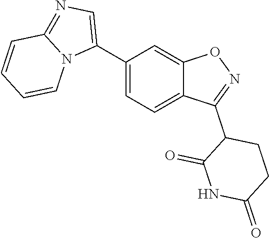 [SMILES: O=C1CCC(c2noc3cc(-c4cnc5ccccn45)ccc23)C(=O)N1] |
| 144. | 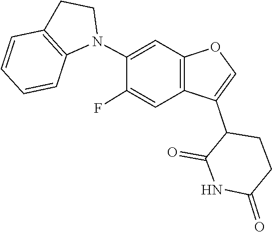 [SMILES: O=C1CCC(c2coc3cc(N4CCc5ccccc54)c(F)cc23)C(=O)N1] |
| 145. | 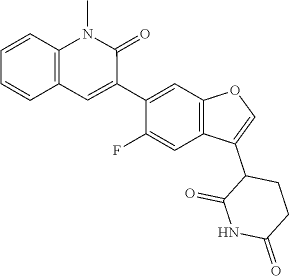 [SMILES: Cn1c(=O)c(-c2cc3occ(C4CCC(=O)NC4=O)c3cc2F)cc2ccccc21] |
| 146. | 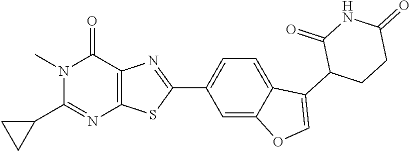 [SMILES: Cn1c(C2CC2)nc2sc(-c3ccc4c(C5CCC(=O)NC5=O)coc4c3)nc2c1=O] |
| 147. | 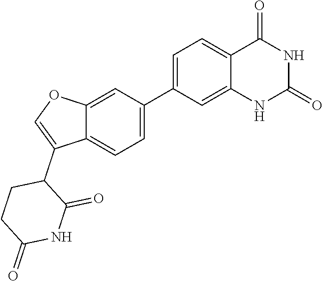 [SMILES: O=C1CCC(c2coc3cc(-c4ccc5c(=O)[nH]c(=O)[nH]c5c4)ccc23)C(=O)N1] |
| 148. | 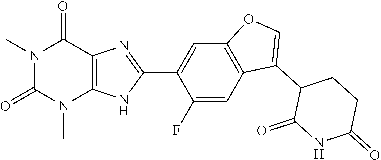 [SMILES: Cn1c(=O)c2nc(-c3cc4occ(C5CCC(=O)NC5=O)c4cc3F)[nH]c2n(C)c1=O] |
| 149. | 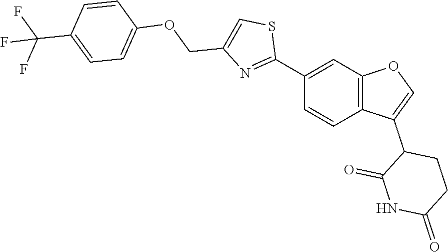 [SMILES: O=C1CCC(c2coc3cc(-c4nc(COc5ccc(C(F)(F)F)cc5)cs4)ccc23)C(=O)N1] |
| 150. | 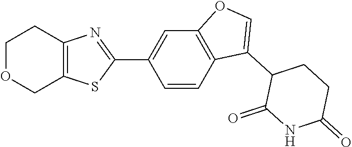 [SMILES: O=C1CCC(c2coc3cc(-c4nc5c(s4)COCC5)ccc23)C(=O)N1] |
| 151. | 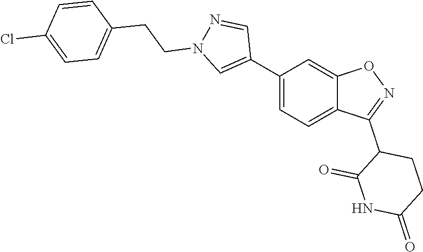 [SMILES: O=C1CCC(c2noc3cc(-c4cnn(CCc5ccc(Cl)cc5)c4)ccc23)C(=O)N1] |
| 152. | 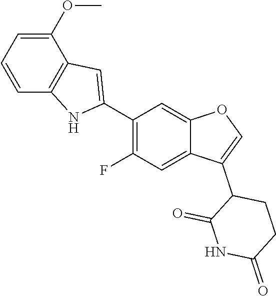 [SMILES: COc1cccc2[nH]c(-c3cc4occ(C5CCC(=O)NC5=O)c4cc3F)cc12] |
| 153. | 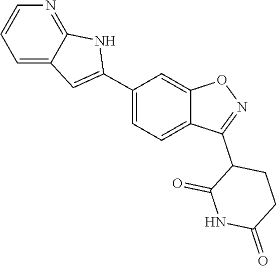 [SMILES: O=C1CCC(c2noc3cc(-c4cc5cccnc5[nH]4)ccc23)C(=O)N1] |
| 154. | 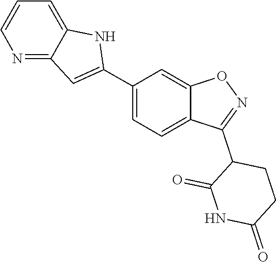 [SMILES: O=C1CCC(c2noc3cc(-c4cc5ncccc5[nH]4)ccc23)C(=O)N1] |
| 155. | 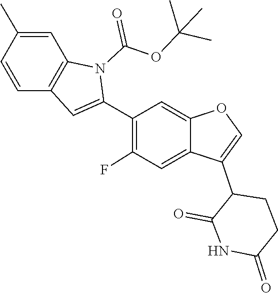 [SMILES: Cc1ccc2cc(-c3cc4occ(C5CCC(=O)NC5=O)c4cc3F)n(C(=O)OC(C)(C)C)c2c1] |
| 156. | 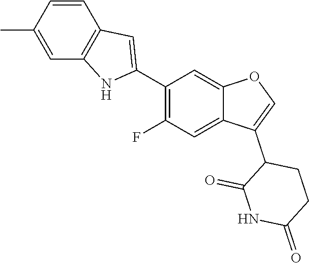 [SMILES: Cc1ccc2cc(-c3cc4occ(C5CCC(=O)NC5=O)c4cc3F)[nH]c2c1] |
| 157. | 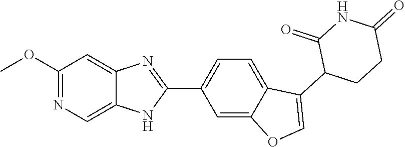 [SMILES: COc1cc2nc(-c3ccc4c(C5CCC(=O)NC5=O)coc4c3)[nH]c2cn1] |
| 158. | 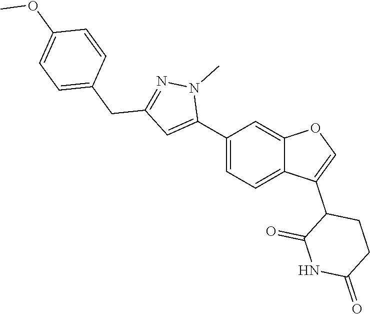 [SMILES: COc1ccc(Cc2cc(-c3ccc4c(C5CCC(=O)NC5=O)coc4c3)n(C)n2)cc1] |
| 159. | 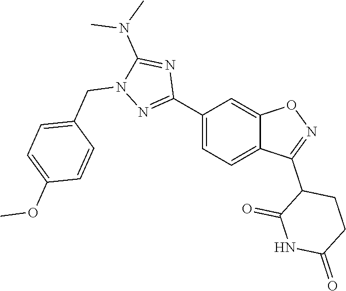 [SMILES: COc1ccc(Cn2nc(-c3ccc4c(C5CCC(=O)NC5=O)noc4c3)nc2N(C)C)cc1] |
| 160. | 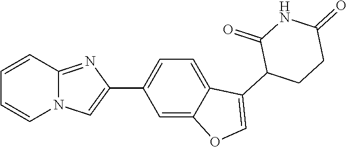 [SMILES: O=C1CCC(c2coc3cc(-c4cn5ccccc5n4)ccc23)C(=O)N1] |
| 161. | 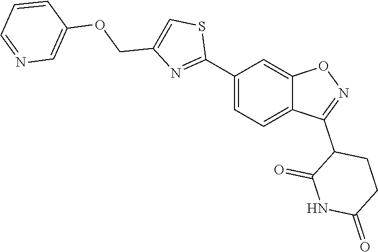 [SMILES: O=C1CCC(c2noc3cc(-c4nc(COc5cccnc5)cs4)ccc23)C(=O)N1] |
| 162. | 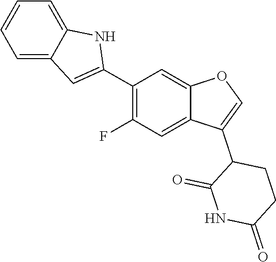 [SMILES: O=C1CCC(c2coc3cc(-c4cc5ccccc5[nH]4)c(F)cc23)C(=O)N1] |
| 163. | 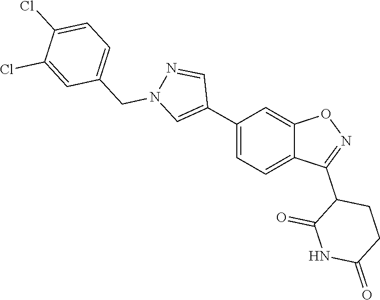 [SMILES: O=C1CCC(c2noc3cc(-c4cnn(Cc5ccc(Cl)c(Cl)c5)c4)ccc23)C(=O)N1] |
| 164. | 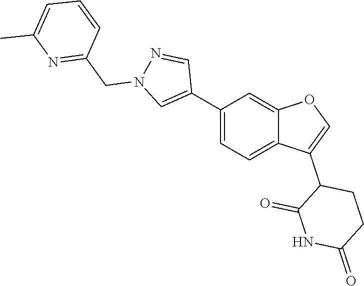 [SMILES: Cc1cccc(Cn2cc(-c3ccc4c(C5CCC(=O)NC5=O)coc4c3)cn2)n1] |
| 165. | 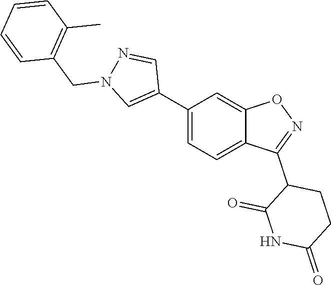 [SMILES: Cc1ccccc1Cn1cc(-c2ccc3c(C4CCC(=O)NC4=O)noc3c2)cn1] |
| 166. | 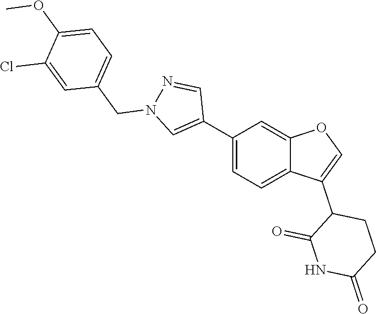 [SMILES: COc1ccc(Cn2cc(-c3ccc4c(C5CCC(=O)NC5=O)coc4c3)cn2)cc1Cl] |
| 167. | 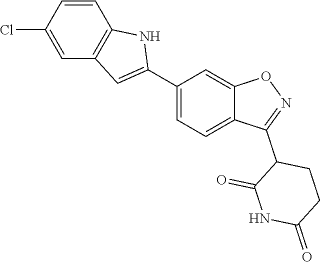 [SMILES: O=C1CCC(c2noc3cc(-c4cc5cc(Cl)ccc5[nH]4)ccc23)C(=O)N1] |
| 168. | 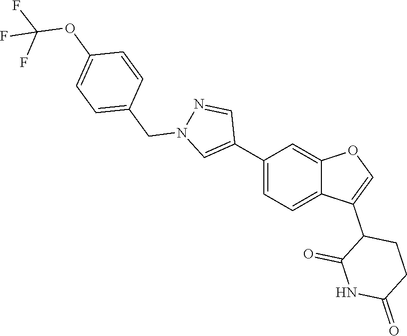 [SMILES: O=C1CCC(c2coc3cc(-c4cnn(Cc5ccc(OC(F)(F)F)cc5)c4)ccc23)C(=O)N1] |
| 169. | 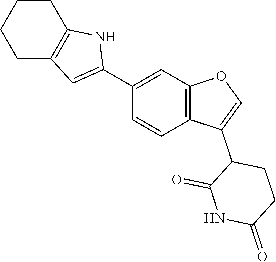 [SMILES: O=C1CCC(c2coc3cc(-c4cc5c([nH]4)CCCC5)ccc23)C(=O)N1] |
| 170. | 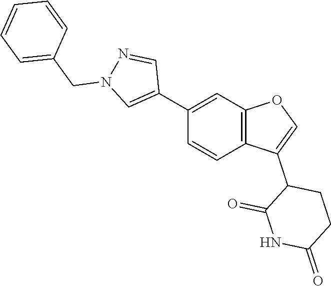 [SMILES: O=C1CCC(c2coc3cc(-c4cnn(Cc5ccccc5)c4)ccc23)C(=O)N1] |
| 171. | 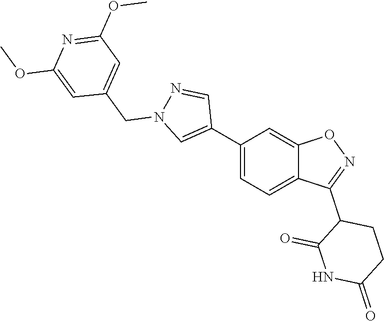 [SMILES: COc1cc(Cn2cc(-c3ccc4c(C5CCC(=O)NC5=O)noc4c3)cn2)cc(OC)n1] |
| 172. | 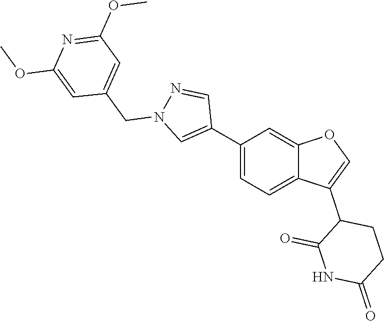 [SMILES: COc1cc(Cn2cc(-c3ccc4c(C5CCC(=O)NC5=O)coc4c3)cn2)cc(OC)n1] |
| 173. | 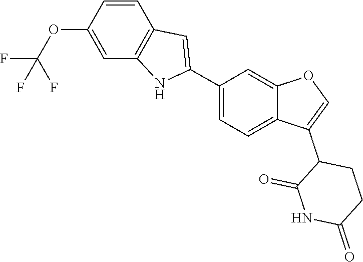 [SMILES: O=C1CCC(c2coc3cc(-c4cc5ccc(OC(F)(F)F)cc5[nH]4)ccc23)C(=O)N1] |
| 174. | 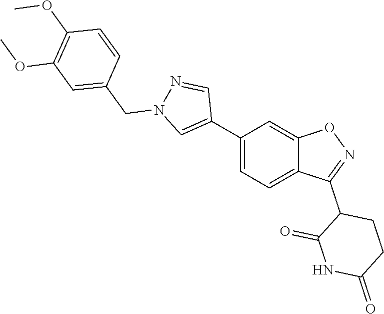 [SMILES: COc1ccc(Cn2cc(-c3ccc4c(C5CCC(=O)NC5=O)noc4c3)cn2)cc1OC] |
| 175. | 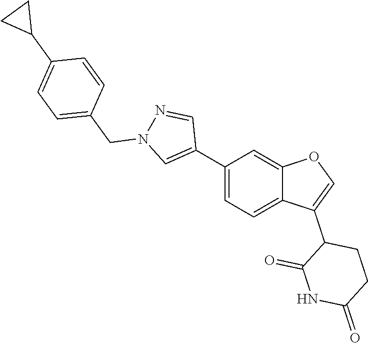 [SMILES: O=C1CCC(c2coc3cc(-c4cnn(Cc5ccc(C6CC6)cc5)c4)ccc23)C(=O)N1] |
| 176. | 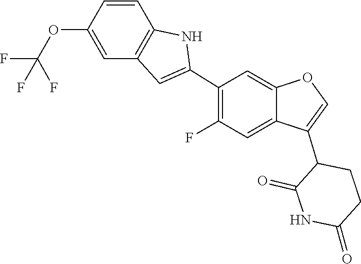 [SMILES: O=C1CCC(c2coc3cc(-c4cc5cc(OC(F)(F)F)ccc5[nH]4)c(F)cc23)C(=O)N1] |
| 177. | 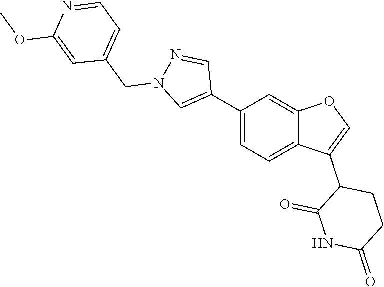 [SMILES: COc1cc(Cn2cc(-c3ccc4c(C5CCC(=O)NC5=O)coc4c3)cn2)ccn1] |
| 178. | 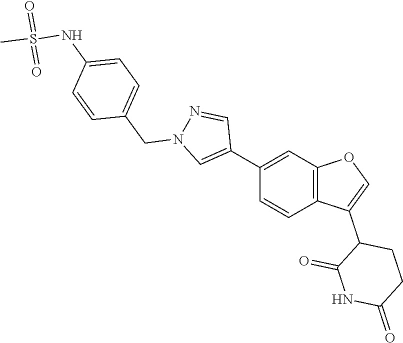 [SMILES: CS(=O)(=O)Nc1ccc(Cn2cc(-c3ccc4c(C5CCC(=O)NC5=O)coc4c3)cn2)cc1] |
| 179. | 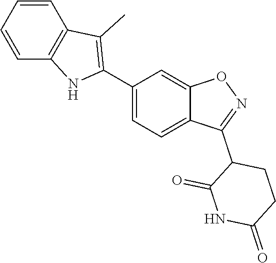 [SMILES: Cc1c(-c2ccc3c(C4CCC(=O)NC4=O)noc3c2)[nH]c2ccccc12] |
| 180. | 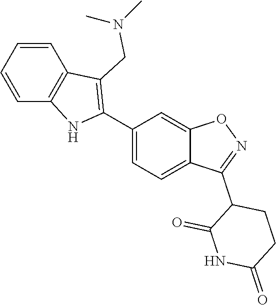 [SMILES: CN(C)Cc1c(-c2ccc3c(C4CCC(=O)NC4=O)noc3c2)[nH]c2ccccc12] |
| 181. | 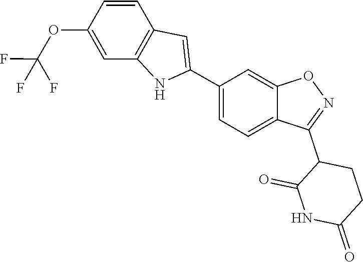 [SMILES: O=C1CCC(c2noc3cc(-c4cc5ccc(OC(F)(F)F)cc5[nH]4)ccc23)C(=O)N1] |
| 182. | 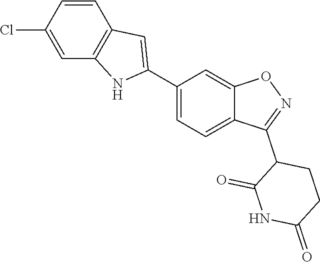 [SMILES: O=C1CCC(c2noc3cc(-c4cc5ccc(Cl)cc5[nH]4)ccc23)C(=O)N1] |
| 183. | 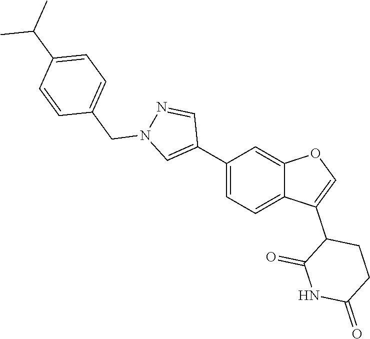 [SMILES: CC(C)c1ccc(Cn2cc(-c3ccc4c(C5CCC(=O)NC5=O)coc4c3)cn2)cc1] |
| 184. | 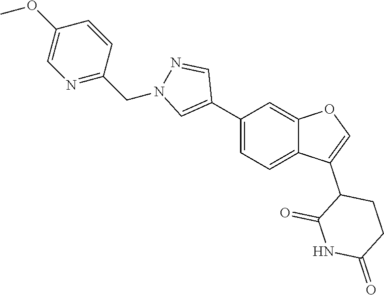 [SMILES: COc1ccc(Cn2cc(-c3ccc4c(C5CCC(=O)NC5=O)coc4c3)cn2)nc1] |
| 185. | 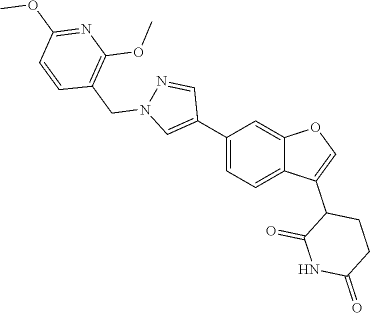 [SMILES: COc1ccc(Cn2cc(-c3ccc4c(C5CCC(=O)NC5=O)coc4c3)cn2)c(OC)n1] |
| 186. | 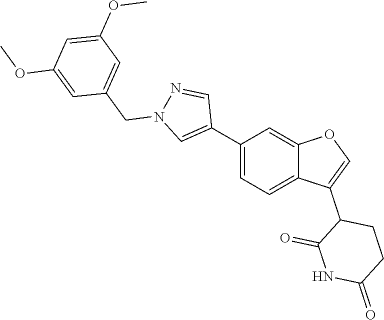 [SMILES: COc1cc(Cn2cc(-c3ccc4c(C5CCC(=O)NC5=O)coc4c3)cn2)cc(OC)c1] |
| 187. | 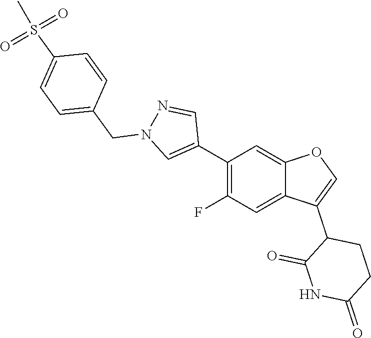 [SMILES: CS(=O)(=O)c1ccc(Cn2cc(-c3cc4occ(C5CCC(=O)NC5=O)c4cc3F)cn2)cc1] |
| 188. | 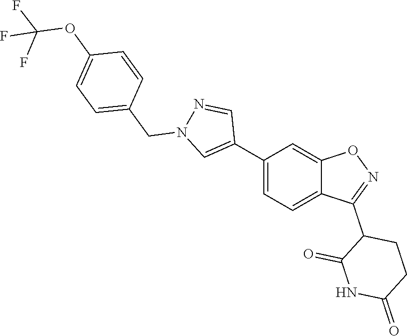 [SMILES: O=C1CCC(c2noc3cc(-c4cnn(Cc5ccc(OC(F)(F)F)cc5)c4)ccc23)C(=O)N1] |
| 189. | 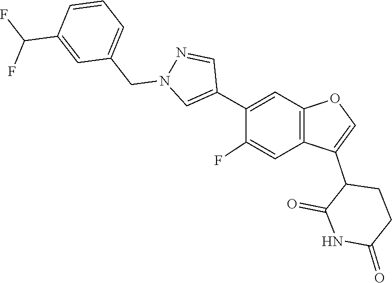 [SMILES: O=C1CCC(c2coc3cc(-c4cnn(Cc5cccc(C(F)F)c5)c4)c(F)cc23)C(=O)N1] |
| 190. | 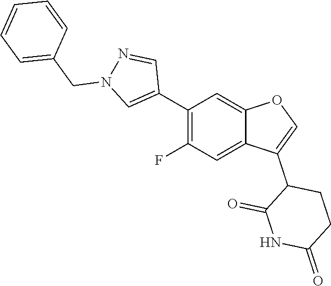 [SMILES: O=C1CCC(c2coc3cc(-c4cnn(Cc5ccccc5)c4)c(F)cc23)C(=O)N1] |
| 191. | 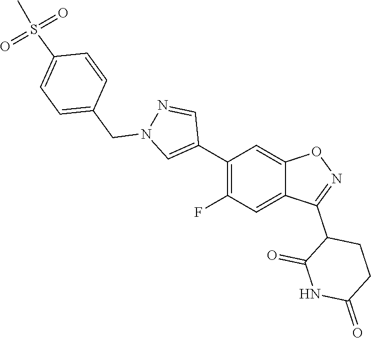 [SMILES: CS(=O)(=O)c1ccc(Cn2cc(-c3cc4onc(C5CCC(=O)NC5=O)c4cc3F)cn2)cc1] |
| 192. | 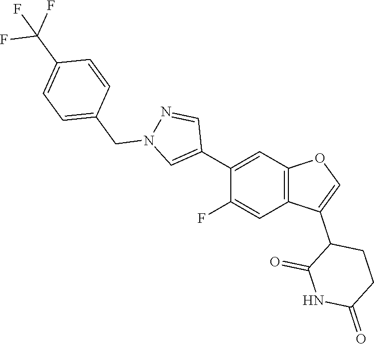 [SMILES: O=C1CCC(c2coc3cc(-c4cnn(Cc5ccc(C(F)(F)F)cc5)c4)c(F)cc23)C(=O)N1] |
| 193. | 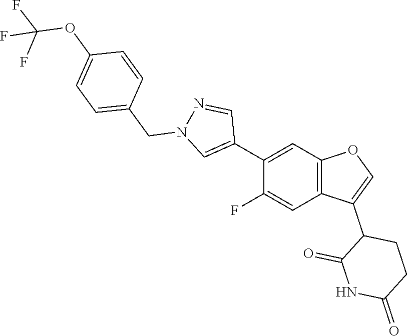 [SMILES: O=C1CCC(c2coc3cc(-c4cnn(Cc5ccc(OC(F)(F)F)cc5)c4)c(F)cc23)C(=O)N1] |
| 194. | 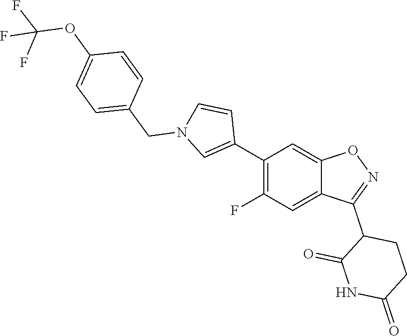 [SMILES: O=C1CCC(c2noc3cc(-c4ccn(Cc5ccc(OC(F)(F)F)cc5)c4)c(F)cc23)C(=O)N1] |
| 195. | 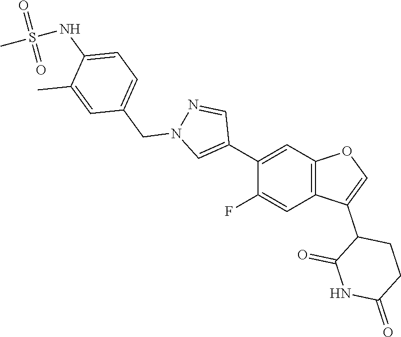 [SMILES: Cc1cc(Cn2cc(-c3cc4occ(C5CCC(=O)NC5=O)c4cc3F)cn2)ccc1NS(C)(=O)=O] |
| 196. | 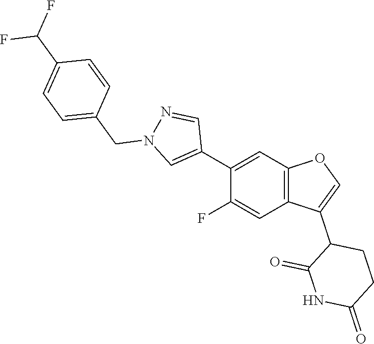 [SMILES: O=C1CCC(c2coc3cc(-c4cnn(Cc5ccc(C(F)F)cc5)c4)c(F)cc23)C(=O)N1] |
| 197. | 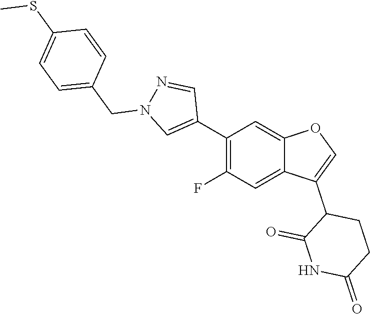 [SMILES: CSc1ccc(Cn2cc(-c3cc4occ(C5CCC(=O)NC5=O)c4cc3F)cn2)cc1] |
| 198. | 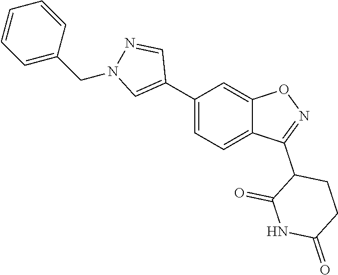 [SMILES: O=C1CCC(c2noc3cc(-c4cnn(Cc5ccccc5)c4)ccc23)C(=O)N1] |
| 199. | 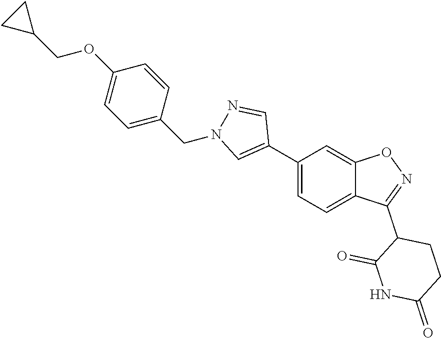 [SMILES: O=C1CCC(c2noc3cc(-c4cnn(Cc5ccc(OCC6CC6)cc5)c4)ccc23)C(=O)N1] |
| 200. | 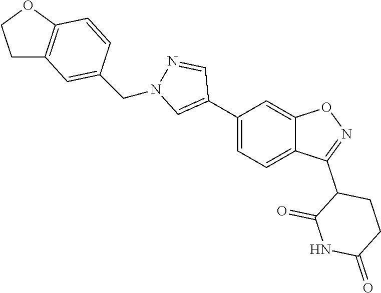 [SMILES: O=C1CCC(c2noc3cc(-c4cnn(Cc5ccc6c(c5)CCO6)c4)ccc23)C(=O)N1] |
| 201. | 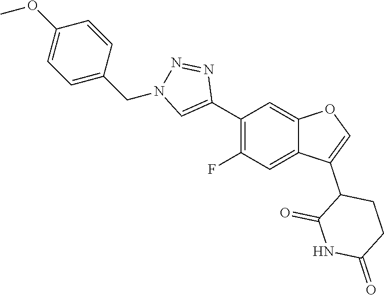 [SMILES: COc1ccc(Cn2cc(-c3cc4occ(C5CCC(=O)NC5=O)c4cc3F)nn2)cc1] |
| 202. | 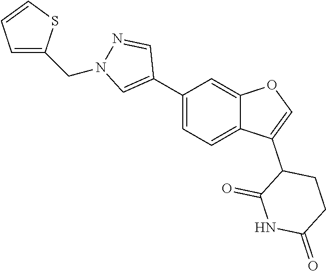 [SMILES: O=C1CCC(c2coc3cc(-c4cnn(Cc5cccs5)c4)ccc23)C(=O)N1] |
| 203. | 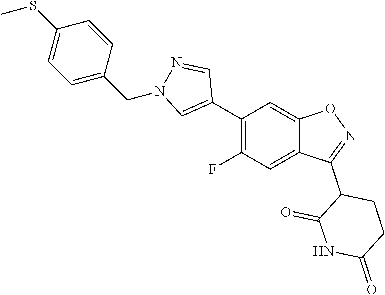 [SMILES: CSc1ccc(Cn2cc(-c3cc4onc(C5CCC(=O)NC5=O)c4cc3F)cn2)cc1] |
| 204. | 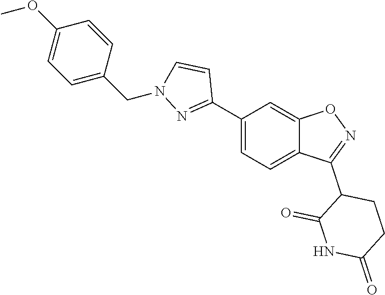 [SMILES: COc1ccc(Cn2ccc(-c3ccc4c(C5CCC(=O)NC5=O)noc4c3)n2)cc1] |
| 205. | 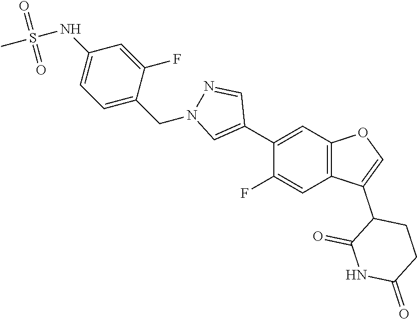 [SMILES: CS(=O)(=O)Nc1ccc(Cn2cc(-c3cc4occ(C5CCC(=O)NC5=O)c4cc3F)cn2)c(F)c1] |
| 206. | 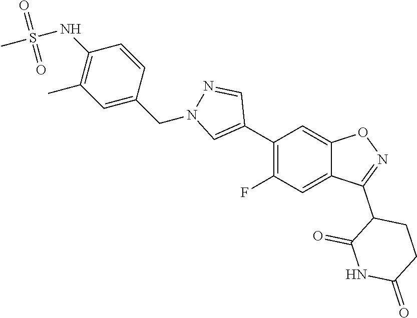 [SMILES: Cc1cc(Cn2cc(-c3cc4onc(C5CCC(=O)NC5=O)c4cc3F)cn2)ccc1NS(C)(=O)=O] |
| 207. | 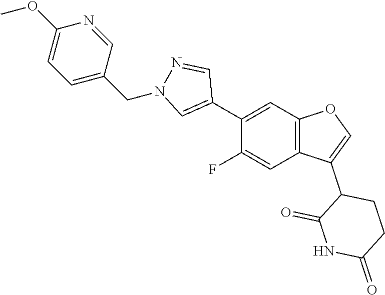 [SMILES: COc1ccc(Cn2cc(-c3cc4occ(C5CCC(=O)NC5=O)c4cc3F)cn2)cn1] |
| 208. | 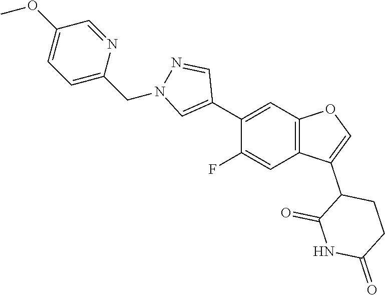 [SMILES: COc1ccc(Cn2cc(-c3cc4occ(C5CCC(=O)NC5=O)c4cc3F)cn2)nc1] |
| 209. | 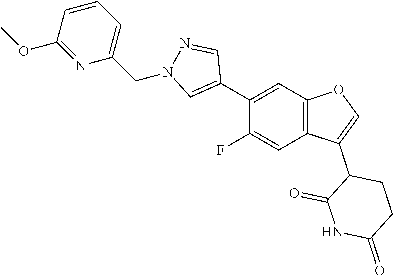 [SMILES: COc1cccc(Cn2cc(-c3cc4occ(C5CCC(=O)NC5=O)c4cc3F)cn2)n1] |
| 210. | 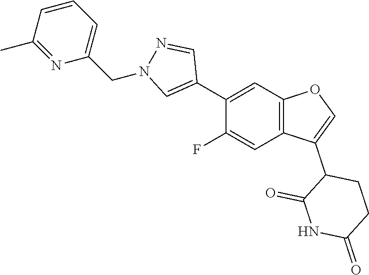 [SMILES: Cc1cccc(Cn2cc(-c3cc4occ(C5CCC(=O)NC5=O)c4cc3F)cn2)n1] |
| 211. | 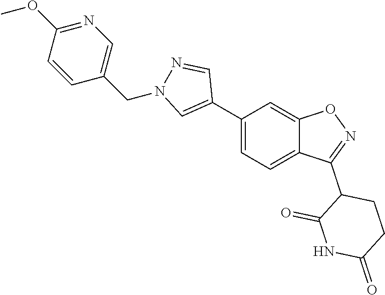 [SMILES: COc1ccc(Cn2cc(-c3ccc4c(C5CCC(=O)NC5=O)noc4c3)cn2)cn1] |
| 212. | 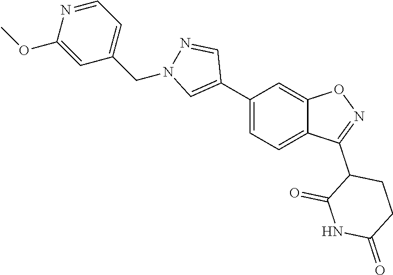 [SMILES: COc1cc(Cn2cc(-c3ccc4c(C5CCC(=O)NC5=O)noc4c3)cn2)ccn1] |
| 213. | 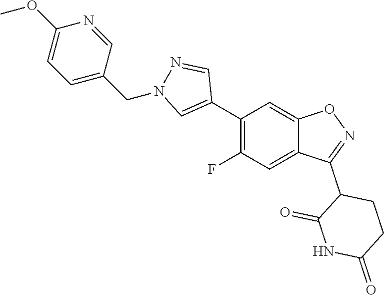 [SMILES: COc1ccc(Cn2cc(-c3cc4onc(C5CCC(=O)NC5=O)c4cc3F)cn2)cn1] |
| 214. | 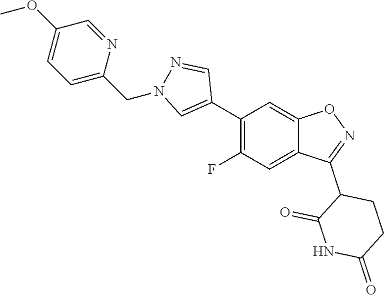 [SMILES: COc1ccc(Cn2cc(-c3cc4onc(C5CCC(=O)NC5=O)c4cc3F)cn2)nc1] |
| 215. | 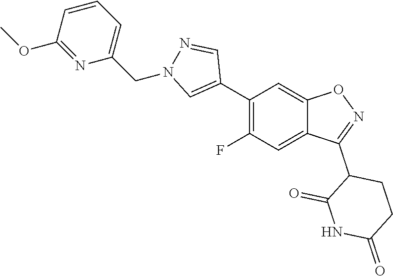 [SMILES: COc1cccc(Cn2cc(-c3cc4onc(C5CCC(=O)NC5=O)c4cc3F)cn2)n1] |
| 216. | 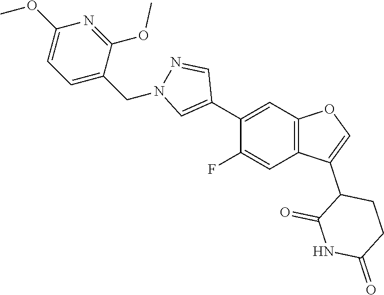 [SMILES: COc1ccc(Cn2cc(-c3cc4occ(C5CCC(=O)NC5=O)c4cc3F)cn2)c(OC)n1] |
| 217. | 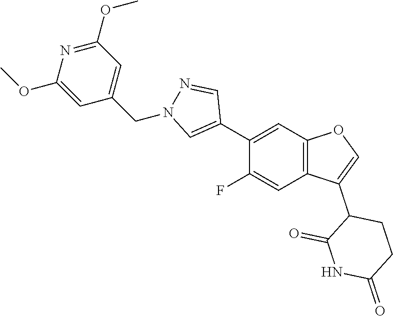 [SMILES: COc1cc(Cn2cc(-c3cc4occ(C5CCC(=O)NC5=O)c4cc3F)cn2)cc(OC)n1] |
| 218. | 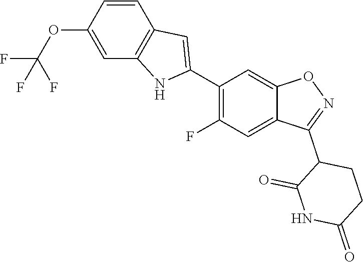 [SMILES: O=C1CCC(c2noc3cc(-c4cc5ccc(OC(F)(F)F)cc5[nH]4)c(F)cc23)C(=O)N1] |
| 219. | 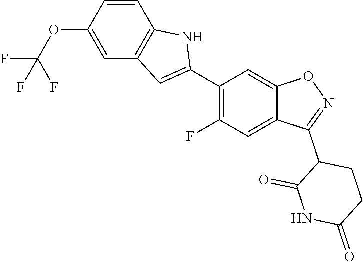 [SMILES: O=C1CCC(c2noc3cc(-c4cc5cc(OC(F)(F)F)ccc5[nH]4)c(F)cc23)C(=O)N1] |
| 220. | 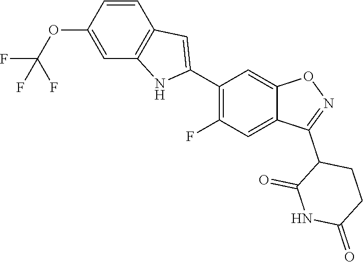 [SMILES: O=C1CCC(c2noc3cc(-c4cc5ccc(OC(F)(F)F)cc5[nH]4)c(F)cc23)C(=O)N1] |
| 221. | 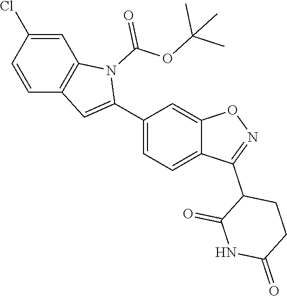 [SMILES: CC(C)(C)OC(=O)n1c(-c2ccc3c(C4CCC(=O)NC4=O)noc3c2)cc2ccc(Cl)cc21] |
| 222. | 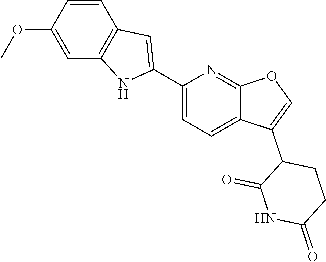 [SMILES: COc1ccc2cc(-c3ccc4c(C5CCC(=O)NC5=O)coc4n3)[nH]c2c1] |
| 223. | 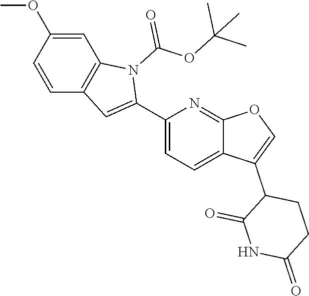 [SMILES: COc1ccc2cc(-c3ccc4c(C5CCC(=O)NC5=O)coc4n3)n(C(=O)OC(C)(C)C)c2c1] |
| 224. | 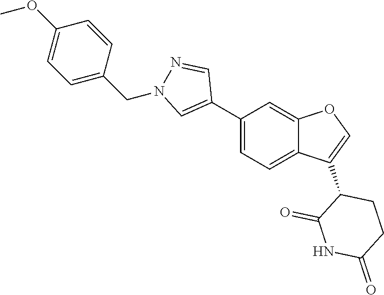 [SMILES: COc1ccc(Cn2cc(-c3ccc4c([C@@H]5CCC(=O)NC5=O)coc4c3)cn2)cc1] |
| 225. | 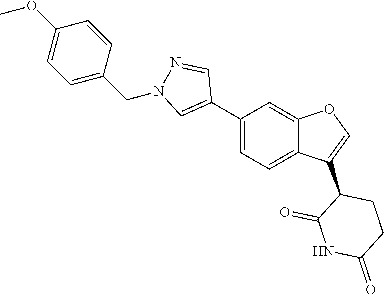 [SMILES: COc1ccc(Cn2cc(-c3ccc4c([C@H]5CCC(=O)NC5=O)coc4c3)cn2)cc1] |
| 226. | 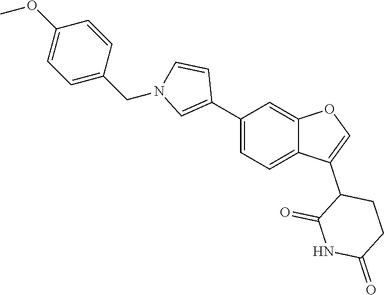 [SMILES: COc1ccc(Cn2ccc(-c3ccc4c(C5CCC(=O)NC5=O)coc4c3)c2)cc1] |
| 227. | 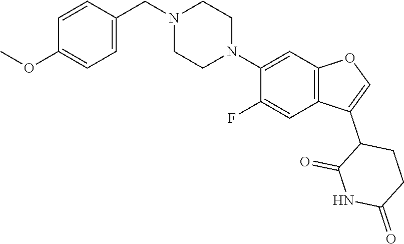 [SMILES: COc1ccc(CN2CCN(c3cc4occ(C5CCC(=O)NC5=O)c4cc3F)CC2)cc1] |
| 228. | 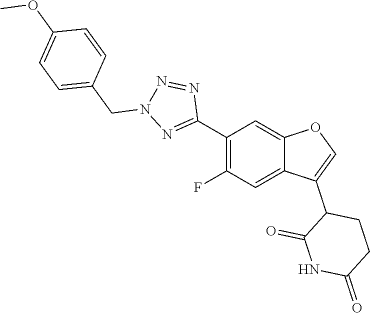 [SMILES: COc1ccc(Cn2nnc(-c3cc4occ(C5CCC(=O)NC5=O)c4cc3F)n2)cc1] |
| 229. | 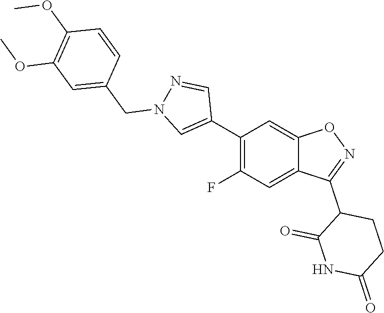 [SMILES: COc1ccc(Cn2cc(-c3cc4onc(C5CCC(=O)NC5=O)c4cc3F)cn2)cc1OC] |
| 230. | 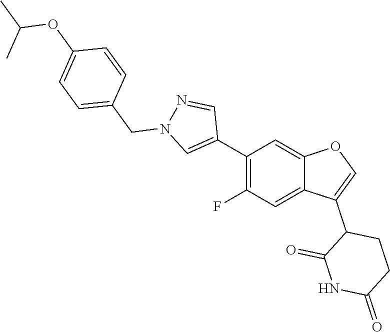 [SMILES: CC(C)Oc1ccc(Cn2cc(-c3cc4occ(C5CCC(=O)NC5=O)c4cc3F)cn2)cc1] |
| 231. | 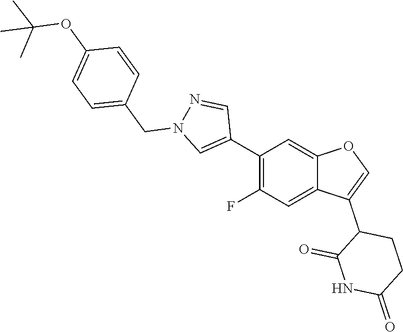 [SMILES: CC(C)(C)Oc1ccc(Cn2cc(-c3cc4occ(C5CCC(=O)NC5=O)c4cc3F)cn2)cc1] |
| 232. | 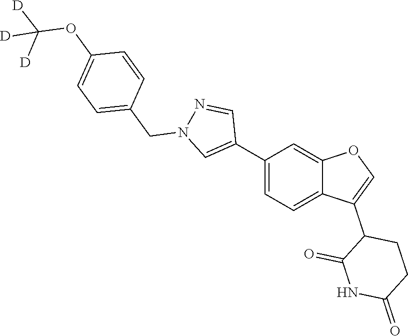 [SMILES: [2H]C([2H])([2H])Oc1ccc(Cn2cc(-c3ccc4c(C5CCC(=O)NC5=O)coc4c3)cn2)cc1] |
| 233. | 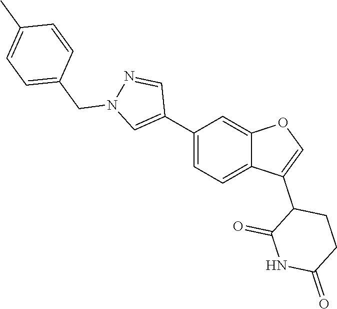 [SMILES: Cc1ccc(Cn2cc(-c3ccc4c(C5CCC(=O)NC5=O)coc4c3)cn2)cc1] |
| 234. | 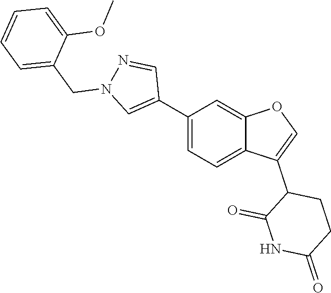 [SMILES: COc1ccccc1Cn1cc(-c2ccc3c(C4CCC(=O)NC4=O)coc3c2)cn1] |
| 235. | 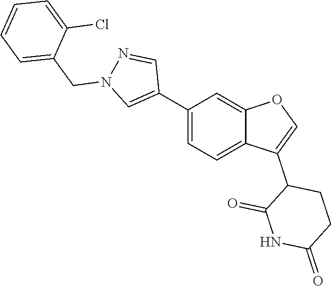 [SMILES: O=C1CCC(c2coc3cc(-c4cnn(Cc5ccccc5Cl)c4)ccc23)C(=O)N1] |
| 236. | 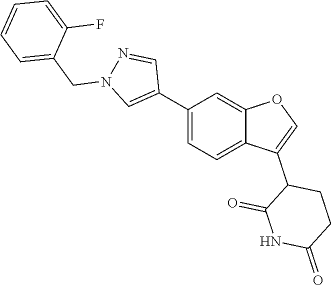 [SMILES: O=C1CCC(c2coc3cc(-c4cnn(Cc5ccccc5F)c4)ccc23)C(=O)N1] |
| 237. | 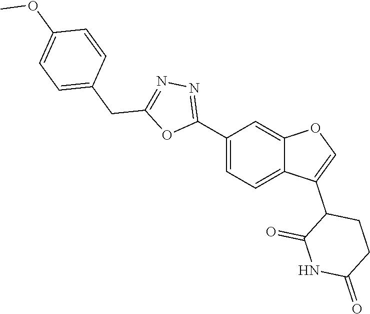 [SMILES: COc1ccc(Cc2nnc(-c3ccc4c(C5CCC(=O)NC5=O)coc4c3)o2)cc1] |
| 238. | 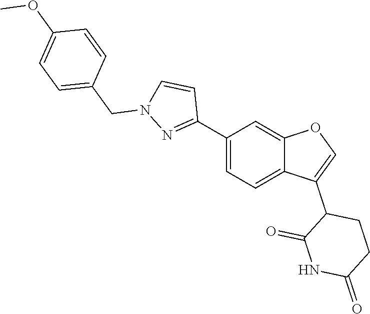 [SMILES: COc1ccc(Cn2ccc(-c3ccc4c(C5CCC(=O)NC5=O)coc4c3)n2)cc1] |
| 239. | 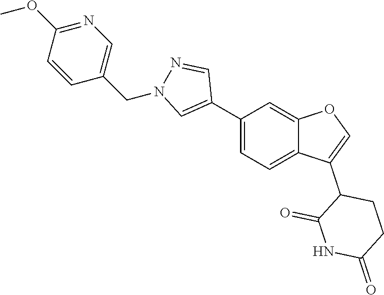 [SMILES: COc1ccc(Cn2cc(-c3ccc4c(C5CCC(=O)NC5=O)coc4c3)cn2)cn1] |
| 240. | 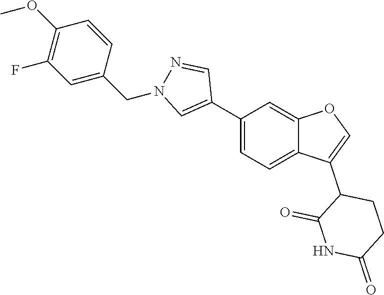 [SMILES: COc1ccc(Cn2cc(-c3ccc4c(C5CCC(=O)NC5=O)coc4c3)cn2)cc1F] |
| 241. | 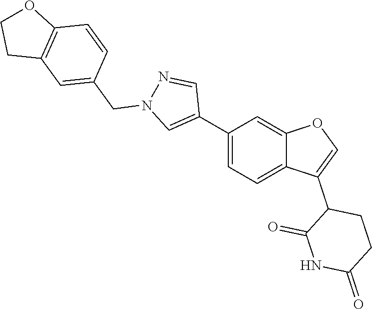 [SMILES: O=C1CCC(c2coc3cc(-c4cnn(Cc5ccc6c(c5)CCO6)c4)ccc23)C(=O)N1] |
| 242. | 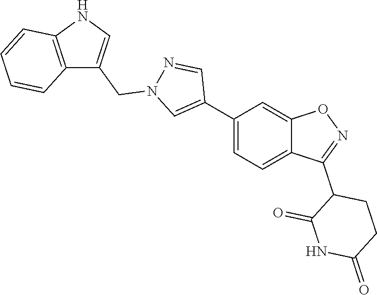 [SMILES: O=C1CCC(c2noc3cc(-c4cnn(Cc5c[nH]c6ccccc56)c4)ccc23)C(=O)N1] |
| 243. | 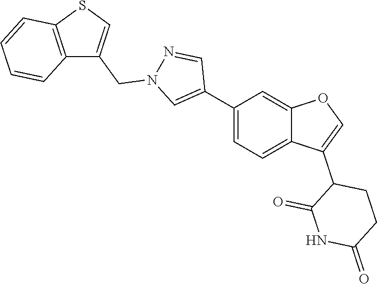 [SMILES: O=C1CCC(c2coc3cc(-c4cnn(Cc5csc6ccccc56)c4)ccc23)C(=O)N1] |
| 244. | 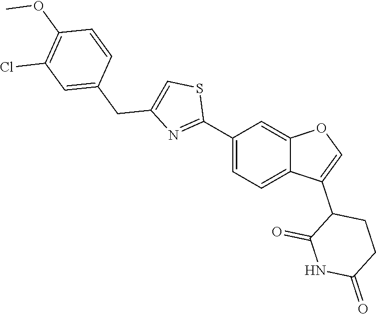 [SMILES: COc1ccc(Cc2csc(-c3ccc4c(C5CCC(=O)NC5=O)coc4c3)n2)cc1Cl] |
| 245. | 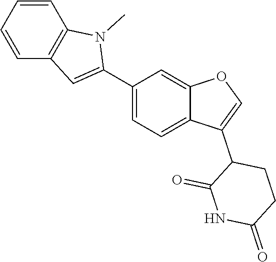 [SMILES: Cn1c(-c2ccc3c(C4CCC(=O)NC4=O)coc3c2)cc2ccccc21] |
| 246. | 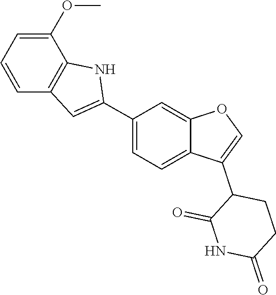 [SMILES: COc1cccc2cc(-c3ccc4c(C5CCC(=O)NC5=O)coc4c3)[nH]c12] |
| 247. | 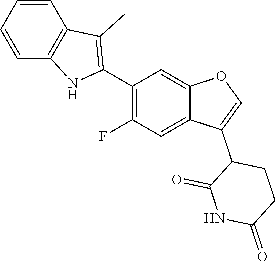 [SMILES: Cc1c(-c2cc3occ(C4CCC(=O)NC4=O)c3cc2F)[nH]c2ccccc12] |
| 248. | 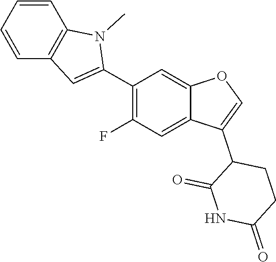 [SMILES: Cn1c(-c2cc3occ(C4CCC(=O)NC4=O)c3cc2F)cc2ccccc21] |
| 249. | 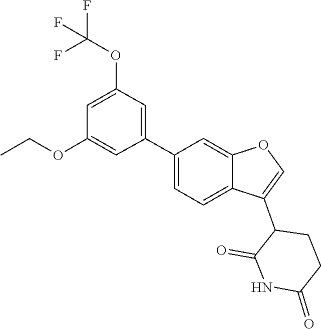 [SMILES: CCOc1cc(OC(F)(F)F)cc(-c2ccc3c(C4CCC(=O)NC4=O)coc3c2)c1] |
| 250. | 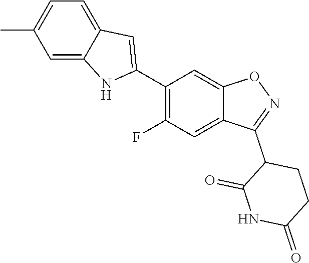 [SMILES: Cc1ccc2cc(-c3cc4onc(C5CCC(=O)NC5=O)c4cc3F)[nH]c2c1] |
| 251. | 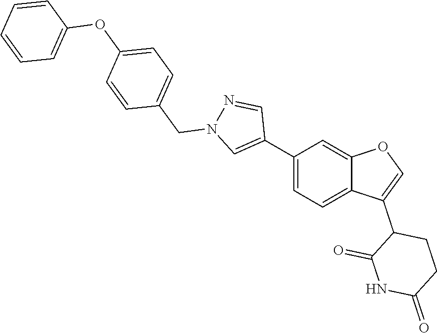 [SMILES: O=C1CCC(c2coc3cc(-c4cnn(Cc5ccc(Oc6ccccc6)cc5)c4)ccc23)C(=O)N1] |
| 252. | 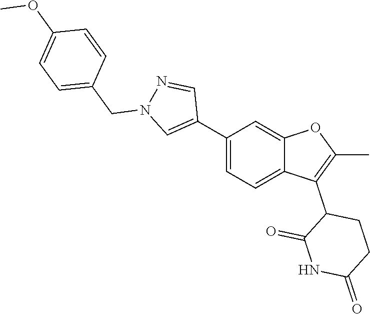 [SMILES: COc1ccc(Cn2cc(-c3ccc4c(C5CCC(=O)NC5=O)c(C)oc4c3)cn2)cc1] |
| 253. | 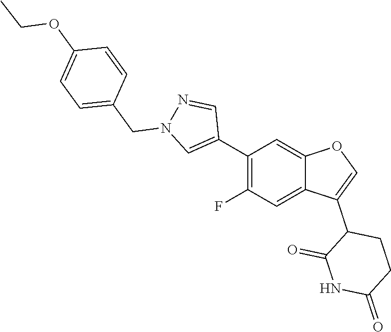 [SMILES: CCOc1ccc(Cn2cc(-c3cc4occ(C5CCC(=O)NC5=O)c4cc3F)cn2)cc1] |
| 254. | 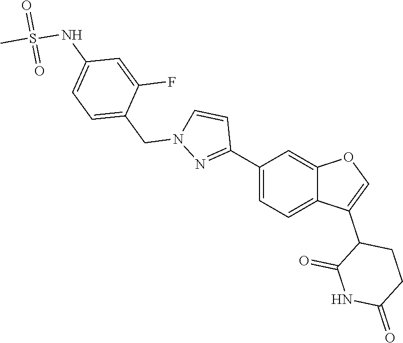 [SMILES: CS(=O)(=O)Nc1ccc(Cn2ccc(-c3ccc4c(C5CCC(=O)NC5=O)coc4c3)n2)c(F)c1] |
| 255. | 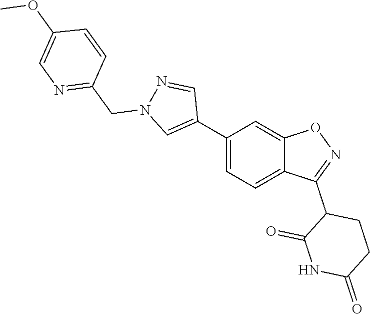 [SMILES: COc1ccc(Cn2cc(-c3ccc4c(C5CCC(=O)NC5=O)noc4c3)cn2)nc1] |
| 256. | 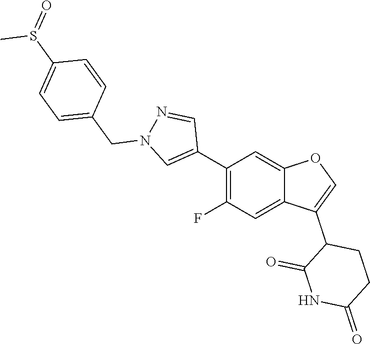 [SMILES: CS(=O)c1ccc(Cn2cc(-c3cc4occ(C5CCC(=O)NC5=O)c4cc3F)cn2)cc1] |
| 257. | 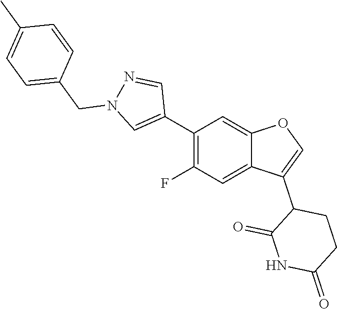 [SMILES: Cc1ccc(Cn2cc(-c3cc4occ(C5CCC(=O)NC5=O)c4cc3F)cn2)cc1] |
| 258. | 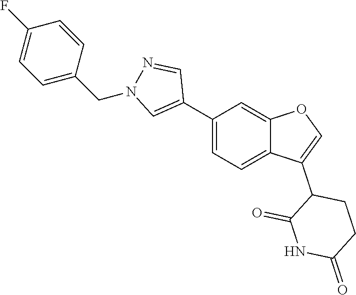 [SMILES: O=C1CCC(c2coc3cc(-c4cnn(Cc5ccc(F)cc5)c4)ccc23)C(=O)N1] |
| 259. | 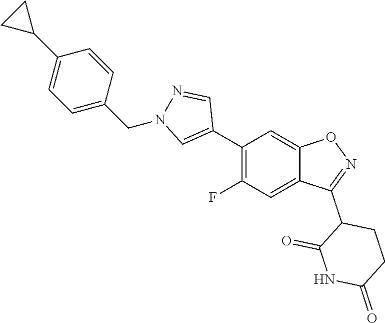 [SMILES: O=C1CCC(c2noc3cc(-c4cnn(Cc5ccc(C6CC6)cc5)c4)c(F)cc23)C(=O)N1] |
| 260. | 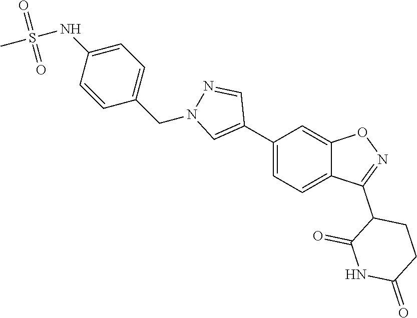 [SMILES: CS(=O)(=O)Nc1ccc(Cn2cc(-c3ccc4c(C5CCC(=O)NC5=O)noc4c3)cn2)cc1] |
| 261. | 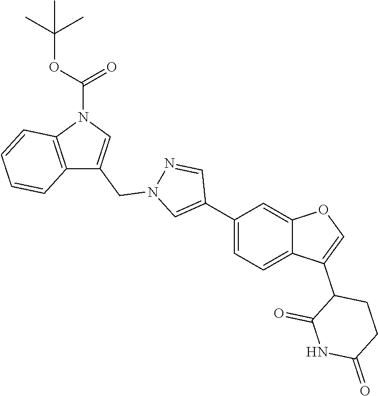 [SMILES: CC(C)(C)OC(=O)n1cc(Cn2cc(-c3ccc4c(C5CCC(=O)NC5=O)coc4c3)cn2)c2ccccc21] |
| 262. | 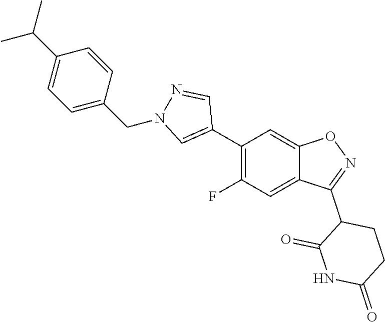 [SMILES: CC(C)c1ccc(Cn2cc(-c3cc4onc(C5CCC(=O)NC5=O)c4cc3F)cn2)cc1] |
| 263. | 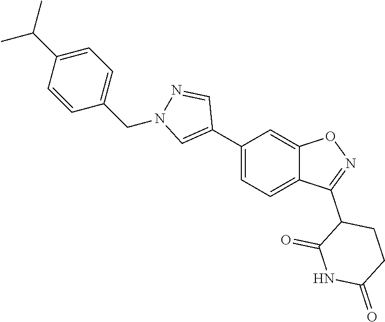 [SMILES: CC(C)c1ccc(Cn2cc(-c3ccc4c(C5CCC(=O)NC5=O)noc4c3)cn2)cc1] |
| 264. | 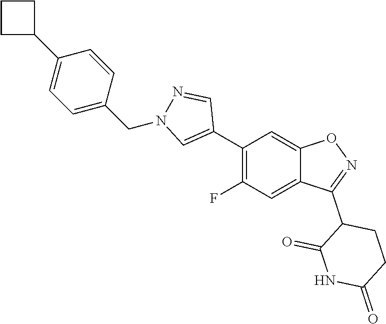 [SMILES: O=C1CCC(c2noc3cc(-c4cnn(Cc5ccc(C6CCC6)cc5)c4)c(F)cc23)C(=O)N1] |
| 265. | 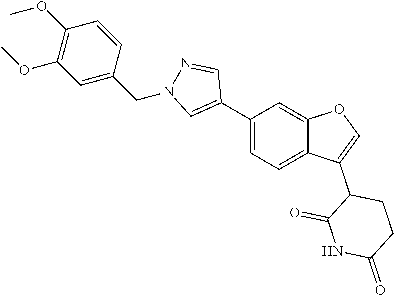 [SMILES: COc1ccc(Cn2cc(-c3ccc4c(C5CCC(=O)NC5=O)coc4c3)cn2)cc1OC] |
| 266. | 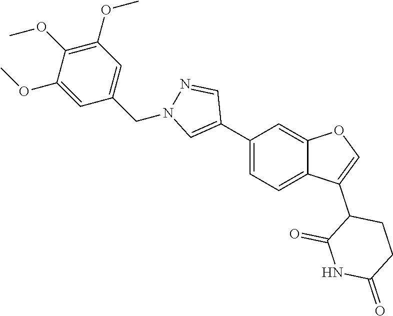 [SMILES: COc1cc(Cn2cc(-c3ccc4c(C5CCC(=O)NC5=O)coc4c3)cn2)cc(OC)c1OC] |
| 267. | 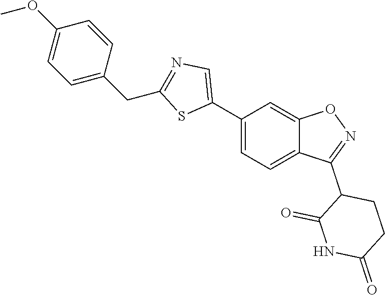 [SMILES: COc1ccc(Cc2ncc(-c3ccc4c(C5CCC(=O)NC5=O)noc4c3)s2)cc1] |
| 268. | 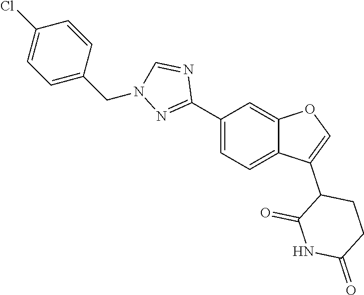 [SMILES: O=C1CCC(c2coc3cc(-c4ncn(Cc5ccc(Cl)cc5)n4)ccc23)C(=O)N1] |
| 269. | 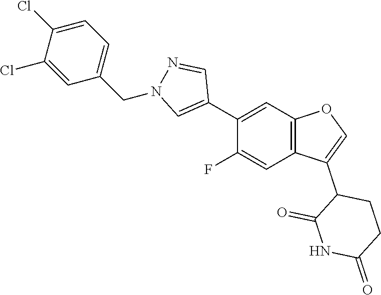 [SMILES: O=C1CCC(c2coc3cc(-c4cnn(Cc5ccc(Cl)c(Cl)c5)c4)c(F)cc23)C(=O)N1] |
| 270. | 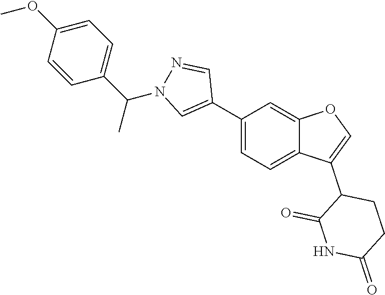 [SMILES: COc1ccc(C(C)n2cc(-c3ccc4c(C5CCC(=O)NC5=O)coc4c3)cn2)cc1] |
| 271. | 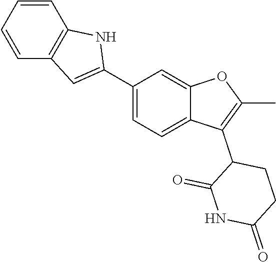 [SMILES: Cc1oc2cc(-c3cc4ccccc4[nH]3)ccc2c1C1CCC(=O)NC1=O] |
| 272. | 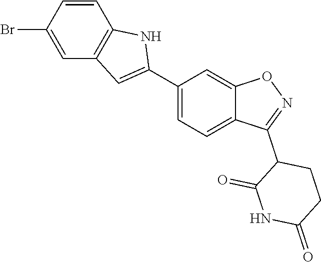 [SMILES: O=C1CCC(c2noc3cc(-c4cc5cc(Br)ccc5[nH]4)ccc23)C(=O)N1] |
| 273. | 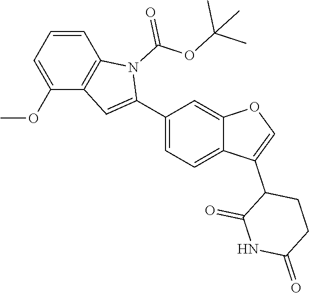 [SMILES: COc1cccc2c1cc(-c1ccc3c(C4CCC(=O)NC4=O)coc3c1)n2C(=O)OC(C)(C)C] |
| 274. | 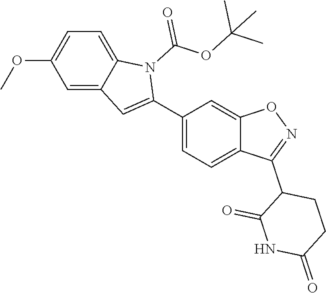 [SMILES: COc1ccc2c(c1)cc(-c1ccc3c(C4CCC(=O)NC4=O)noc3c1)n2C(=O)OC(C)(C)C] |
| 275. | 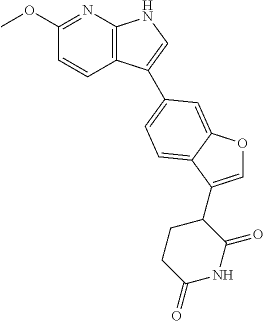 [SMILES: COc1ccc2c(-c3ccc4c(C5CCC(=O)NC5=O)coc4c3)c[nH]c2n1] |
| 276. | 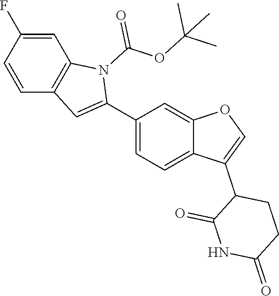 [SMILES: CC(C)(C)OC(=O)n1c(-c2ccc3c(C4CCC(=O)NC4=O)coc3c2)cc2ccc(F)cc21] |
| 277. | 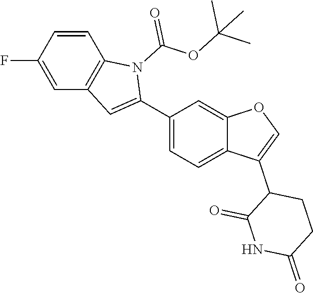 [SMILES: CC(C)(C)OC(=O)n1c(-c2ccc3c(C4CCC(=O)NC4=O)coc3c2)cc2cc(F)ccc21] |
| 278. | 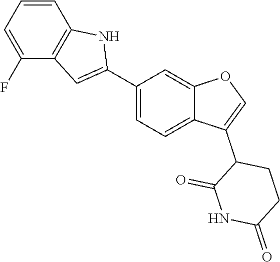 [SMILES: O=C1CCC(c2coc3cc(-c4cc5c(F)cccc5[nH]4)ccc23)C(=O)N1] |
| 279. | 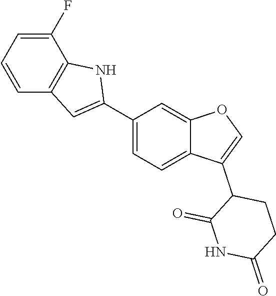 [SMILES: O=C1CCC(c2coc3cc(-c4cc5cccc(F)c5[nH]4)ccc23)C(=O)N1] |
| 280. | 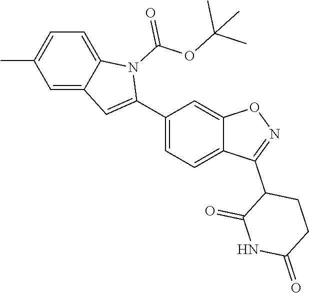 [SMILES: Cc1ccc2c(c1)cc(-c1ccc3c(C4CCC(=O)NC4=O)noc3c1)n2C(=O)OC(C)(C)C] |
| 281. | 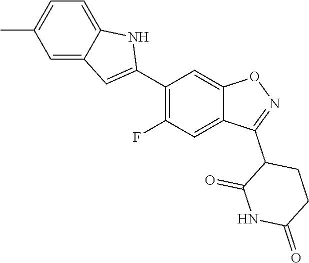 [SMILES: Cc1ccc2[nH]c(-c3cc4onc(C5CCC(=O)NC5=O)c4cc3F)cc2c1] |
| 282. | 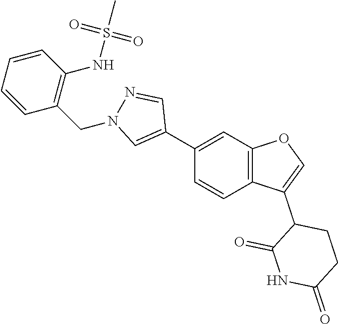 [SMILES: CS(=O)(=O)Nc1ccccc1Cn1cc(-c2ccc3c(C4CCC(=O)NC4=O)coc3c2)cn1] |
| 283. | 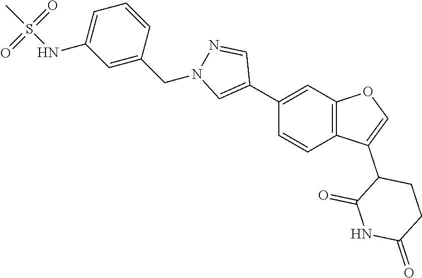 [SMILES: CS(=O)(=O)Nc1cccc(Cn2cc(-c3ccc4c(C5CCC(=O)NC5=O)coc4c3)cn2)c1] |
| 284. | 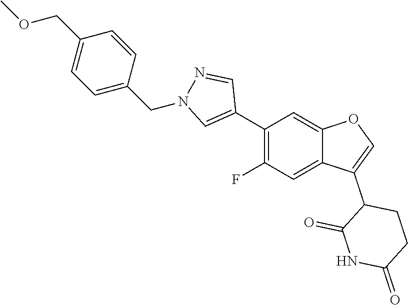 [SMILES: COCc1ccc(Cn2cc(-c3cc4occ(C5CCC(=O)NC5=O)c4cc3F)cn2)cc1] |
| 285. | 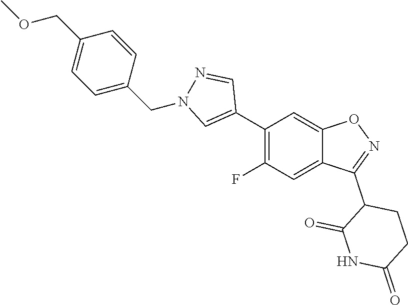 [SMILES: COCc1ccc(Cn2cc(-c3cc4onc(C5CCC(=O)NC5=O)c4cc3F)cn2)cc1] |
| 286. | 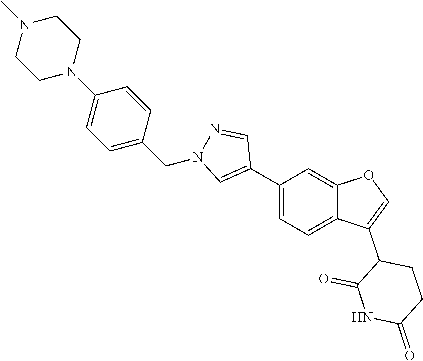 [SMILES: CN1CCN(c2ccc(Cn3cc(-c4ccc5c(C6CCC(=O)NC6=O)coc5c4)cn3)cc2)CC1] |
| 287. | 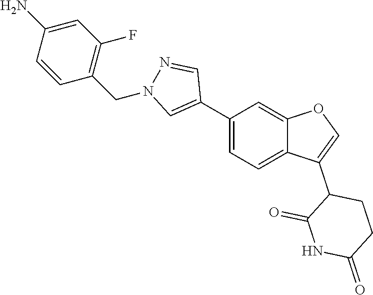 [SMILES: Nc1ccc(Cn2cc(-c3ccc4c(C5CCC(=O)NC5=O)coc4c3)cn2)c(F)c1] |
| 288. | 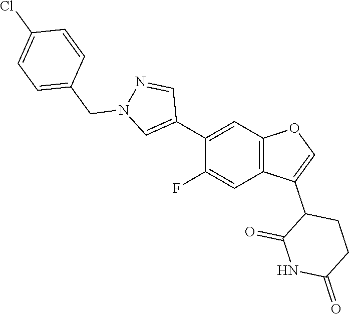 [SMILES: O=C1CCC(c2coc3cc(-c4cnn(Cc5ccc(Cl)cc5)c4)c(F)cc23)C(=O)N1] |
| 289. | 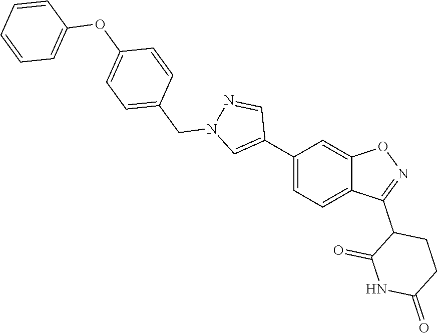 [SMILES: O=C1CCC(c2noc3cc(-c4cnn(Cc5ccc(Oc6ccccc6)cc5)c4)ccc23)C(=O)N1] |
| 290. | 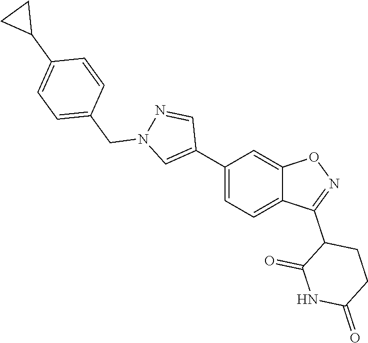 [SMILES: O=C1CCC(c2noc3cc(-c4cnn(Cc5ccc(C6CC6)cc5)c4)ccc23)C(=O)N1] |
| 291. | 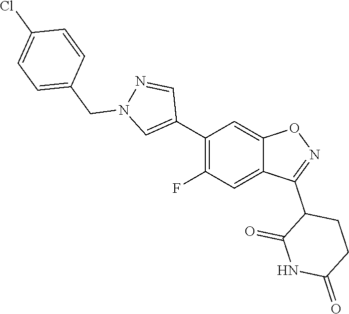 [SMILES: O=C1CCC(c2noc3cc(-c4cnn(Cc5ccc(Cl)cc5)c4)c(F)cc23)C(=O)N1] |
| 292. | 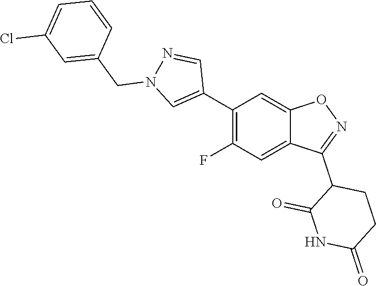 [SMILES: O=C1CCC(c2noc3cc(-c4cnn(Cc5cccc(Cl)c5)c4)c(F)cc23)C(=O)N1] |
| 293. | 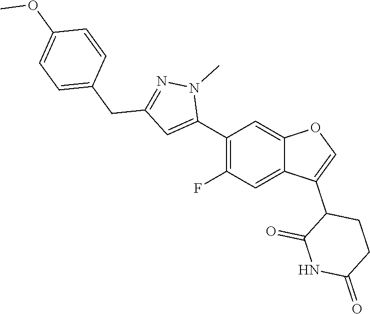 [SMILES: COc1ccc(Cc2cc(-c3cc4occ(C5CCC(=O)NC5=O)c4cc3F)n(C)n2)cc1] |
| 294. | 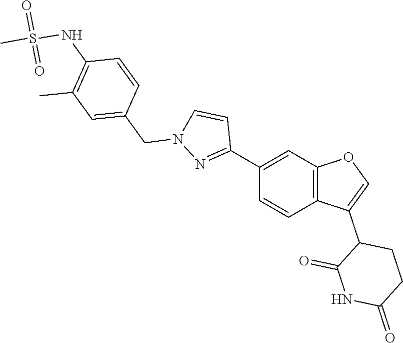 [SMILES: Cc1cc(Cn2ccc(-c3ccc4c(C5CCC(=O)NC5=O)coc4c3)n2)ccc1NS(C)(=O)=O] |
| 295. | 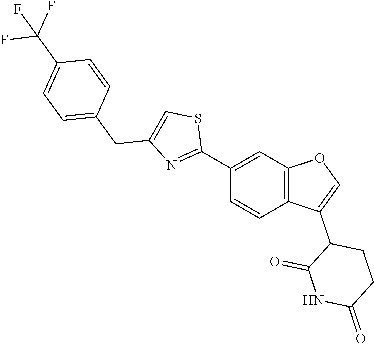 [SMILES: O=C1CCC(c2coc3cc(-c4nc(Cc5ccc(C(F)(F)F)cc5)cs4)ccc23)C(=O)N1] |
| 296. | 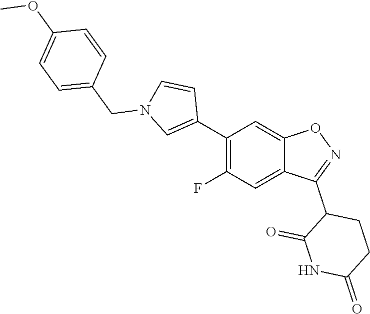 [SMILES: COc1ccc(Cn2ccc(-c3cc4onc(C5CCC(=O)NC5=O)c4cc3F)c2)cc1] |
| 297. | 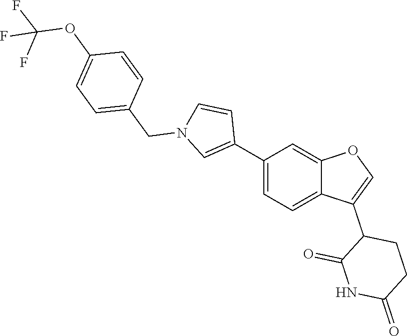 [SMILES: O=C1CCC(c2coc3cc(-c4ccn(Cc5ccc(OC(F)(F)F)cc5)c4)ccc23)C(=O)N1] |
| 298. | 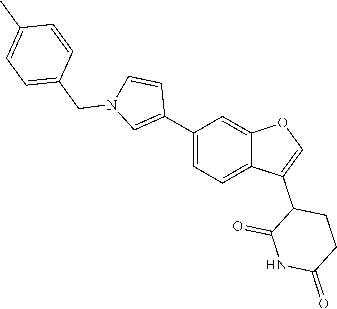 [SMILES: Cc1ccc(Cn2ccc(-c3ccc4c(C5CCC(=O)NC5=O)coc4c3)c2)cc1] |
| 299. | 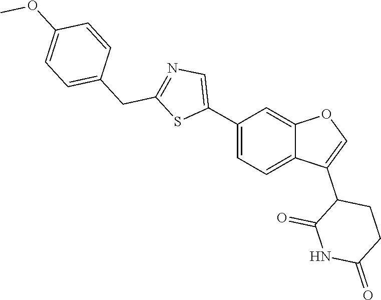 [SMILES: COc1ccc(Cc2ncc(-c3ccc4c(C5CCC(=O)NC5=O)coc4c3)s2)cc1] |
| 300. | 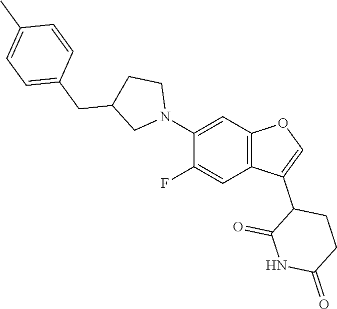 [SMILES: Cc1ccc(CC2CCN(c3cc4occ(C5CCC(=O)NC5=O)c4cc3F)C2)cc1] |
| 301. | 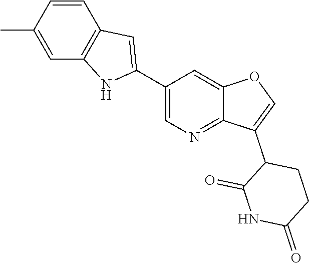 [SMILES: Cc1ccc2cc(-c3cnc4c(C5CCC(=O)NC5=O)coc4c3)[nH]c2c1] |
| 302. | 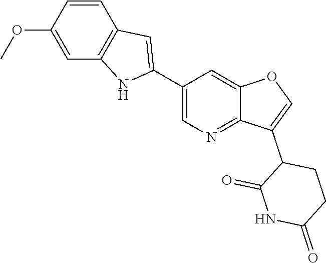 [SMILES: COc1ccc2cc(-c3cnc4c(C5CCC(=O)NC5=O)coc4c3)[nH]c2c1] |
| 303. | 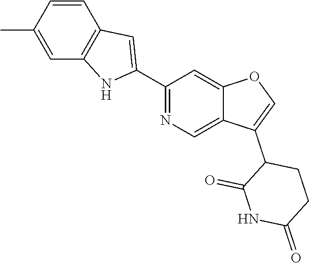 [SMILES: Cc1ccc2cc(-c3cc4occ(C5CCC(=O)NC5=O)c4cn3)[nH]c2c1] |
| 304. | 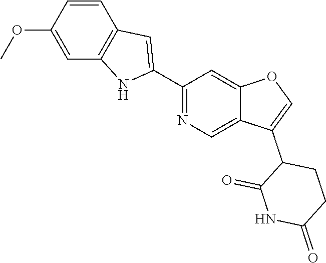 [SMILES: COc1ccc2cc(-c3cc4occ(C5CCC(=O)NC5=O)c4cn3)[nH]c2c1] |
| 305. | 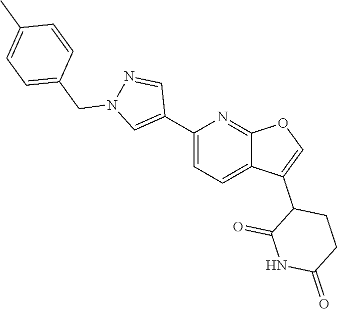 [SMILES: Cc1ccc(Cn2cc(-c3ccc4c(C5CCC(=O)NC5=O)coc4n3)cn2)cc1] |
| 306. | 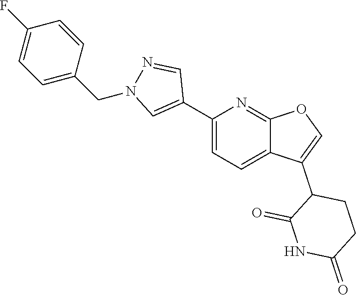 [SMILES: O=C1CCC(c2coc3nc(-c4cnn(Cc5ccc(F)cc5)c4)ccc23)C(=O)N1] |
| 307. | 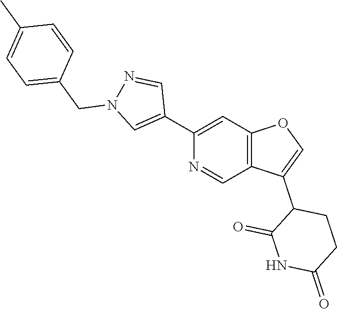 [SMILES: Cc1ccc(Cn2cc(-c3cc4occ(C5CCC(=O)NC5=O)c4cn3)cn2)cc1] |
| 308. | 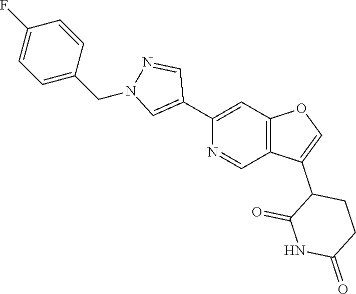 [SMILES: O=C1CCC(c2coc3cc(-c4cnn(Cc5ccc(F)cc5)c4)ncc23)C(=O)N1] |
| 309. | 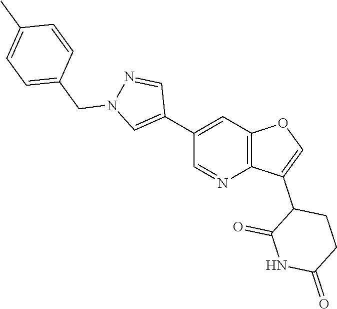 [SMILES: Cc1ccc(Cn2cc(-c3cnc4c(C5CCC(=O)NC5=O)coc4c3)cn2)cc1] |
| 310. | 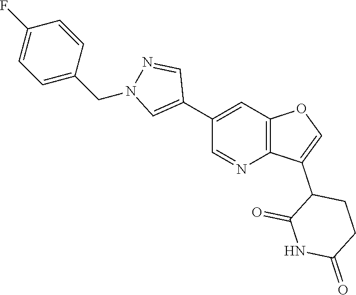 [SMILES: O=C1CCC(c2coc3cc(-c4cnn(Cc5ccc(F)cc5)c4)cnc23)C(=O)N1] |
| 311. | 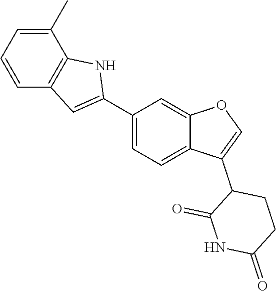 [SMILES: Cc1cccc2cc(-c3ccc4c(C5CCC(=O)NC5=O)coc4c3)[nH]c12] |
| 312. | 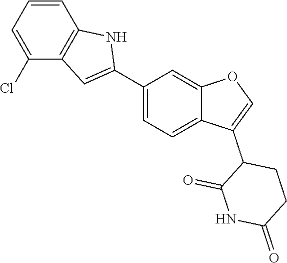 [SMILES: O=C1CCC(c2coc3cc(-c4cc5c(Cl)cccc5[nH]4)ccc23)C(=O)N1] |
| 313. | 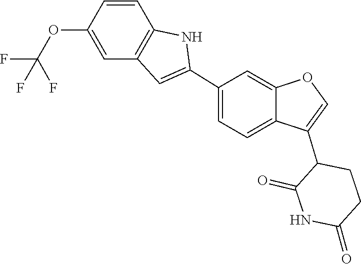 [SMILES: O=C1CCC(c2coc3cc(-c4cc5cc(OC(F)(F)F)ccc5[nH]4)ccc23)C(=O)N1] |
| 314. | 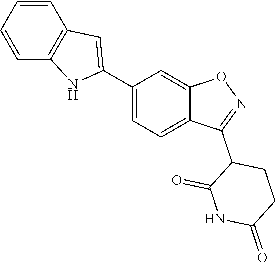 [SMILES: O=C1CCC(c2noc3cc(-c4cc5ccccc5[nH]4)ccc23)C(=O)N1] |
| 315. | 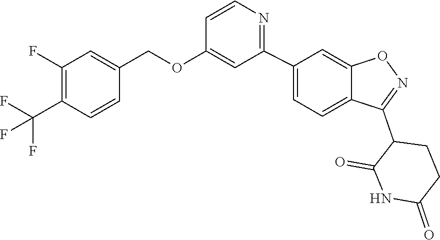 [SMILES: O=C1CCC(c2noc3cc(-c4cc(OCc5ccc(C(F)(F)F)c(F)c5)ccn4)ccc23)C(=O)N1] |
| 316. | 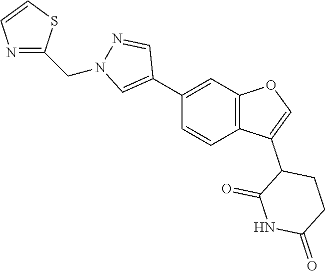 [SMILES: O=C1CCC(c2coc3cc(-c4cnn(Cc5nccs5)c4)ccc23)C(=O)N1] |
| 317. | 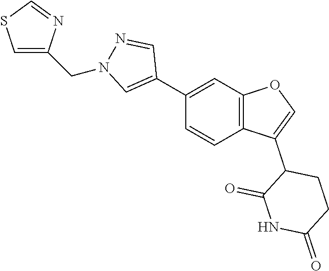 [SMILES: O=C1CCC(c2coc3cc(-c4cnn(Cc5cscn5)c4)ccc23)C(=O)N1] |
| 318. | 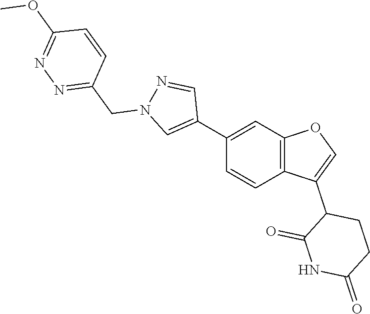 [SMILES: COc1ccc(Cn2cc(-c3ccc4c(C5CCC(=O)NC5=O)coc4c3)cn2)nn1] |
| 319. | 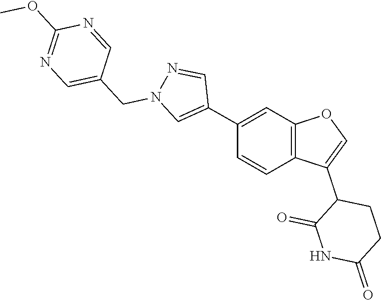 [SMILES: COc1ncc(Cn2cc(-c3ccc4c(C5CCC(=O)NC5=O)coc4c3)cn2)cn1] |
| 320. | 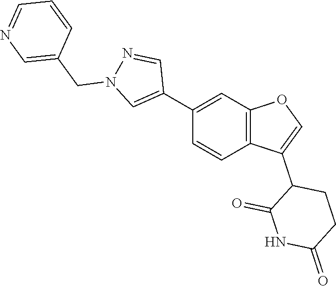 [SMILES: O=C1CCC(c2coc3cc(-c4cnn(Cc5cccnc5)c4)ccc23)C(=O)N1] |
| 321. | 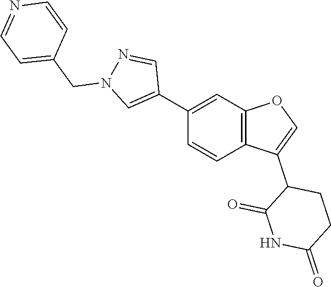 [SMILES: O=C1CCC(c2coc3cc(-c4cnn(Cc5ccncc5)c4)ccc23)C(=O)N1] |
| 322. | 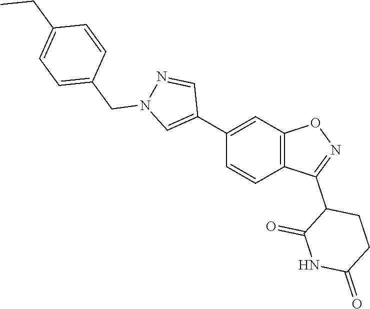 [SMILES: CCc1ccc(Cn2cc(-c3ccc4c(C5CCC(=O)NC5=O)noc4c3)cn2)cc1] |
| 323. | 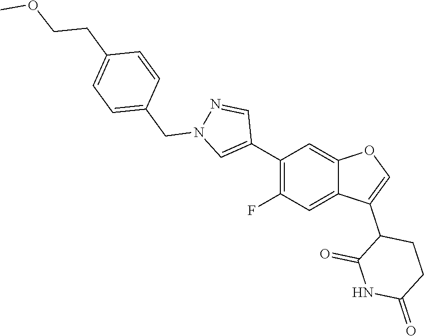 [SMILES: COCCc1ccc(Cn2cc(-c3cc4occ(C5CCC(=O)NC5=O)c4cc3F)cn2)cc1] |
| 324. | 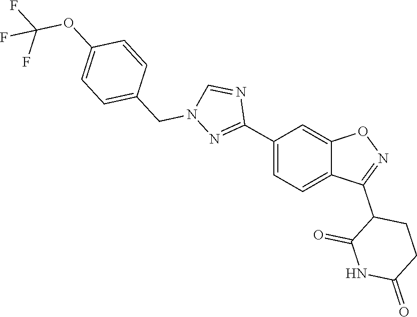 [SMILES: O=C1CCC(c2noc3cc(-c4ncn(Cc5ccc(OC(F)(F)F)cc5)n4)ccc23)C(=O)N1] |
| 325. | 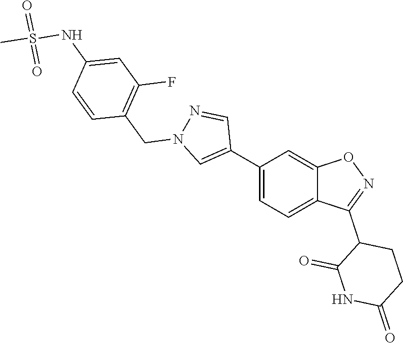 [SMILES: CS(=O)(=O)Nc1ccc(Cn2cc(-c3ccc4c(C5CCC(=O)NC5=O)noc4c3)cn2)c(F)c1] |
| 326. | 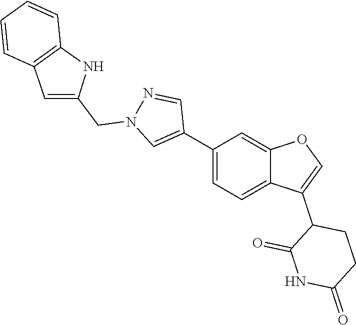 [SMILES: O=C1CCC(c2coc3cc(-c4cnn(Cc5cc6ccccc6[nH]5)c4)ccc23)C(=O)N1] |
| 327. | 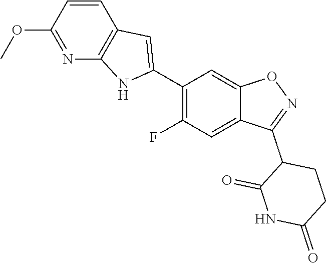 [SMILES: COc1ccc2cc(-c3cc4onc(C5CCC(=O)NC5=O)c4cc3F)[nH]c2n1] |
| 328. | 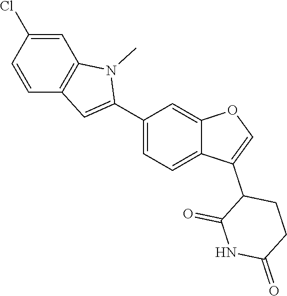 [SMILES: Cn1c(-c2ccc3c(C4CCC(=O)NC4=O)coc3c2)cc2ccc(Cl)cc21] |
| 329. | 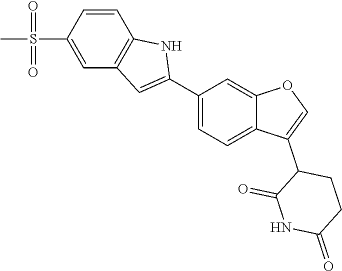 [SMILES: CS(=O)(=O)c1ccc2[nH]c(-c3ccc4c(C5CCC(=O)NC5=O)coc4c3)cc2c1] |
| 330. | 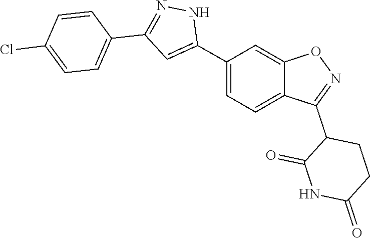 [SMILES: O=C1CCC(c2noc3cc(-c4cc(-c5ccc(Cl)cc5)n[nH]4)ccc23)C(=O)N1] |
| 331. | 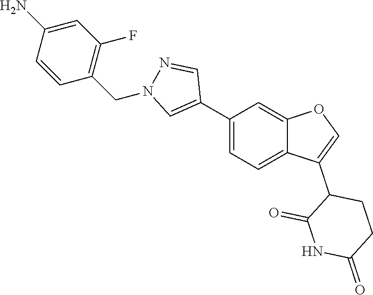 [SMILES: Nc1ccc(Cn2cc(-c3ccc4c(C5CCC(=O)NC5=O)coc4c3)cn2)c(F)c1] |
| 332. | 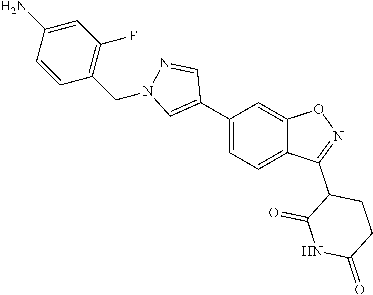 [SMILES: Nc1ccc(Cn2cc(-c3ccc4c(C5CCC(=O)NC5=O)noc4c3)cn2)c(F)c1] |
| 333. | 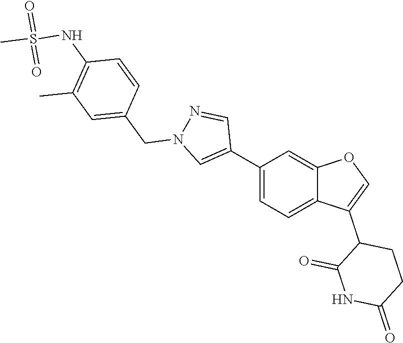 [SMILES: Cc1cc(Cn2cc(-c3ccc4c(C5CCC(=O)NC5=O)coc4c3)cn2)ccc1NS(C)(=O)=O] |
| 334. | 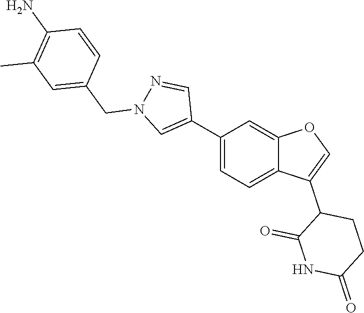 [SMILES: Cc1cc(Cn2cc(-c3ccc4c(C5CCC(=O)NC5=O)coc4c3)cn2)ccc1N] |
| 335. | 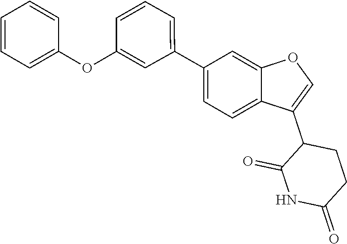 [SMILES: O=C1CCC(c2coc3cc(-c4cccc(Oc5ccccc5)c4)ccc23)C(=O)N1] |
| 336. | 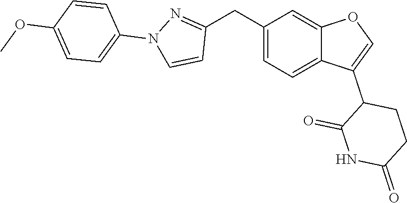 [SMILES: COc1ccc(-n2ccc(Cc3ccc4c(C5CCC(=O)NC5=O)coc4c3)n2)cc1] |
| 337. | 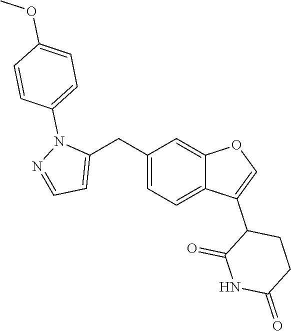 [SMILES: COc1ccc(-n2nccc2Cc2ccc3c(C4CCC(=O)NC4=O)coc3c2)cc1] |
| 338. | 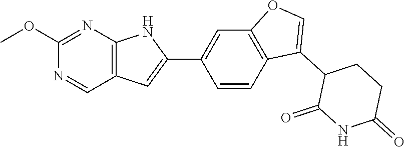 [SMILES: COc1ncc2cc(-c3ccc4c(C5CCC(=O)NC5=O)coc4c3)[nH]c2n1] |
| 339. | 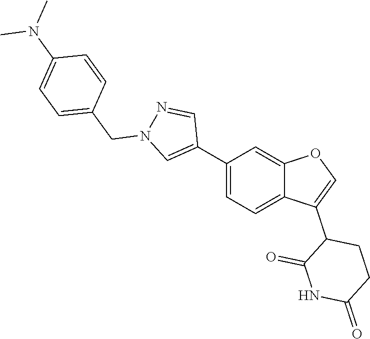 [SMILES: CN(C)c1ccc(Cn2cc(-c3ccc4c(C5CCC(=O)NC5=O)coc4c3)cn2)cc1] |
| 340. | 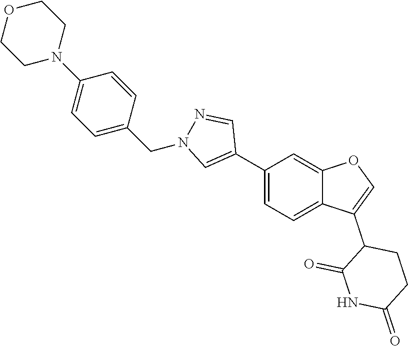 [SMILES: O=C1CCC(c2coc3cc(-c4cnn(Cc5ccc(N6CCOCC6)cc5)c4)ccc23)C(=O)N1] |
| 341. | 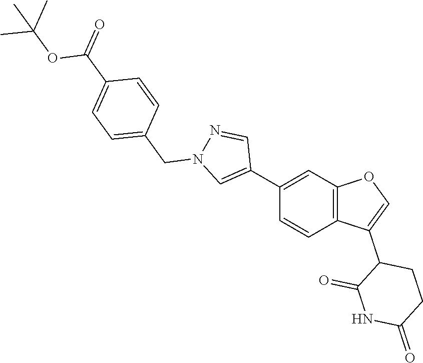 [SMILES: CC(C)(C)OC(=O)c1ccc(Cn2cc(-c3ccc4c(C5CCC(=O)NC5=O)coc4c3)cn2)cc1] |
| 342. | 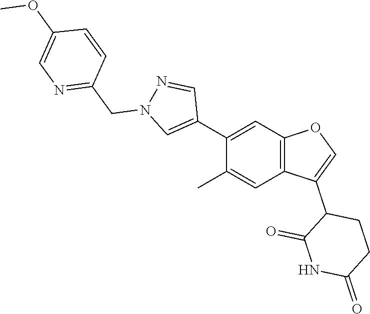 [SMILES: COc1ccc(Cn2cc(-c3cc4occ(C5CCC(=O)NC5=O)c4cc3C)cn2)nc1] |
| 343. | 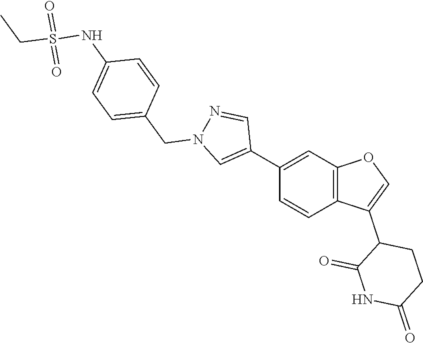 [SMILES: CCS(=O)(=O)Nc1ccc(Cn2cc(-c3ccc4c(C5CCC(=O)NC5=O)coc4c3)cn2)cc1] |
| 344. | 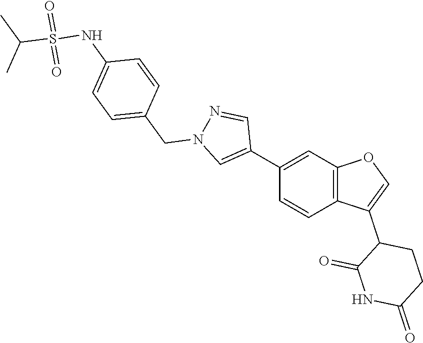 [SMILES: CC(C)S(=O)(=O)Nc1ccc(Cn2cc(-c3ccc4c(C5CCC(=O)NC5=O)coc4c3)cn2)cc1] |
| 345. | 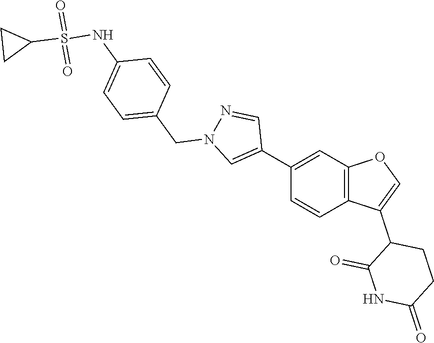 [SMILES: O=C1CCC(c2coc3cc(-c4cnn(Cc5ccc(NS(=O)(=O)C6CC6)cc5)c4)ccc23)C(=O)N1] |
| 346. | 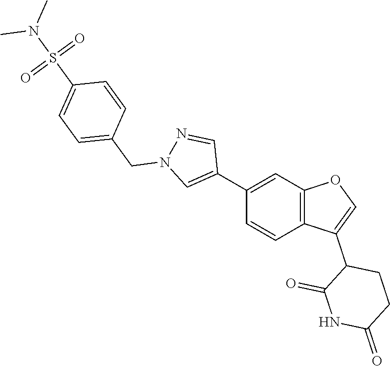 [SMILES: CN(C)S(=O)(=O)c1ccc(Cn2cc(-c3ccc4c(C5CCC(=O)NC5=O)coc4c3)cn2)cc1] |
| 347. | 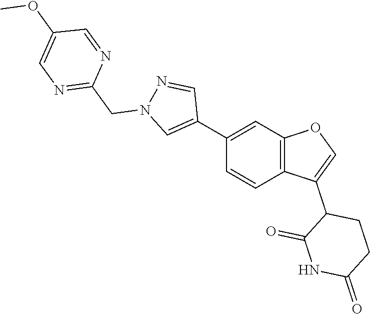 [SMILES: COc1cnc(Cn2cc(-c3ccc4c(C5CCC(=O)NC5=O)coc4c3)cn2)nc1] |
| 348. | 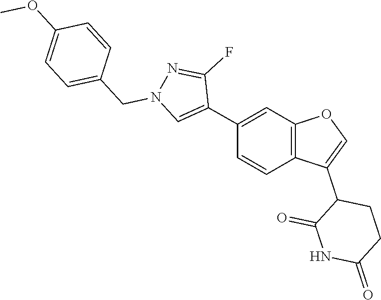 [SMILES: COc1ccc(Cn2cc(-c3ccc4c(C5CCC(=O)NC5=O)coc4c3)c(F)n2)cc1] |
| 349. | 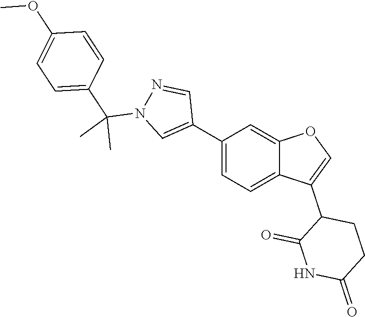 [SMILES: COc1ccc(C(C)(C)n2cc(-c3ccc4c(C5CCC(=O)NC5=O)coc4c3)cn2)cc1] |
| 350. | 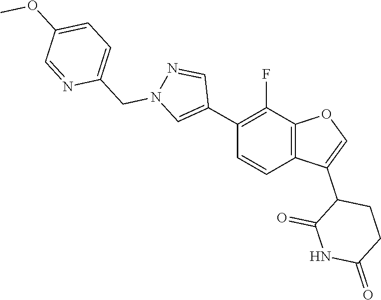 [SMILES: COc1ccc(Cn2cc(-c3ccc4c(C5CCC(=O)NC5=O)coc4c3F)cn2)nc1] |
| 351. | 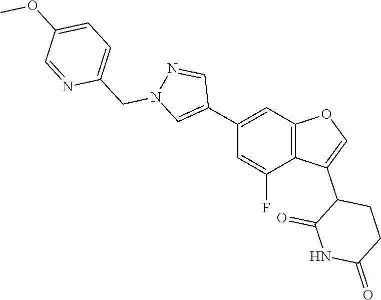 [SMILES: COc1ccc(Cn2cc(-c3cc(F)c4c(C5CCC(=O)NC5=O)coc4c3)cn2)nc1] |
| 352. | 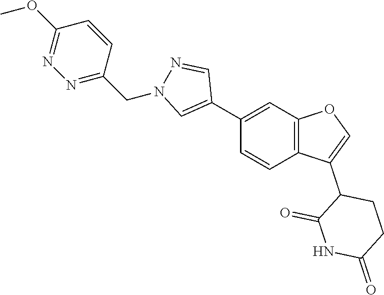 [SMILES: COc1ccc(Cn2cc(-c3ccc4c(C5CCC(=O)NC5=O)coc4c3)cn2)nn1] |
| 353. | 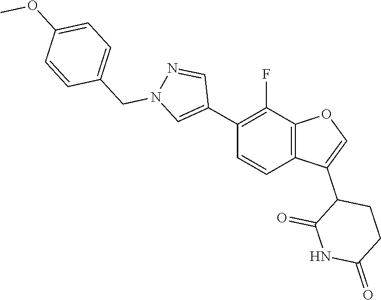 [SMILES: COc1ccc(Cn2cc(-c3ccc4c(C5CCC(=O)NC5=O)coc4c3F)cn2)cc1] |
| 354. | 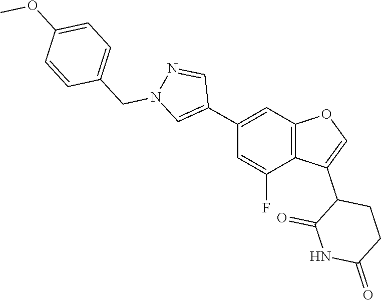 [SMILES: COc1ccc(Cn2cc(-c3cc(F)c4c(C5CCC(=O)NC5=O)coc4c3)cn2)cc1] |
| 355. | 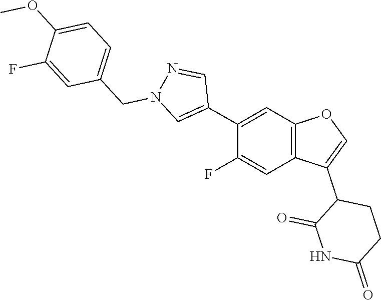 [SMILES: COc1ccc(Cn2cc(-c3cc4occ(C5CCC(=O)NC5=O)c4cc3F)cn2)cc1F] |
| 356. | 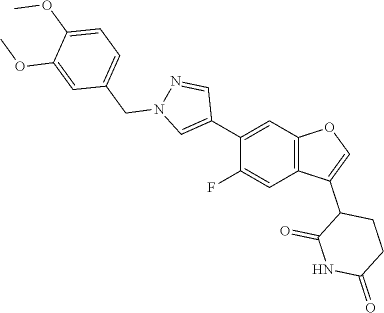 [SMILES: COc1ccc(Cn2cc(-c3cc4occ(C5CCC(=O)NC5=O)c4cc3F)cn2)cc1OC] |
| 357. | 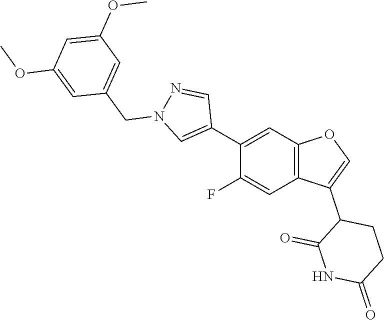 [SMILES: COc1cc(Cn2cc(-c3cc4occ(C5CCC(=O)NC5=O)c4cc3F)cn2)cc(OC)c1] |
| 358. | 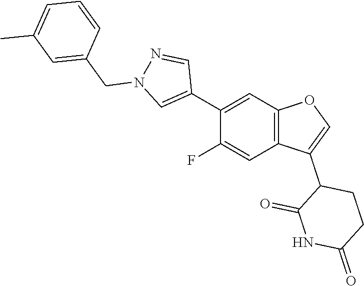 [SMILES: Cc1cccc(Cn2cc(-c3cc4occ(C5CCC(=O)NC5=O)c4cc3F)cn2)c1] |
| 359. | 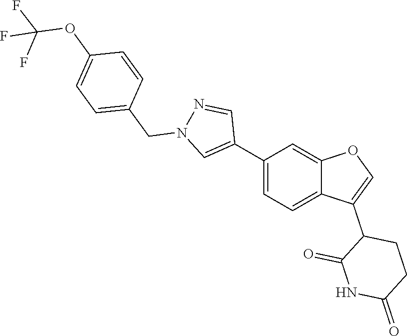 [SMILES: O=C1CCC(c2coc3cc(-c4cnn(Cc5ccc(OC(F)(F)F)cc5)c4)ccc23)C(=O)N1] |
| 360. | 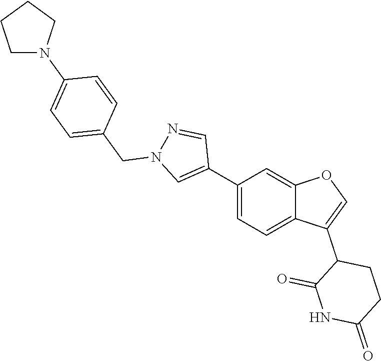 [SMILES: O=C1CCC(c2coc3cc(-c4cnn(Cc5ccc(N6CCCC6)cc5)c4)ccc23)C(=O)N1] |
| 361. | 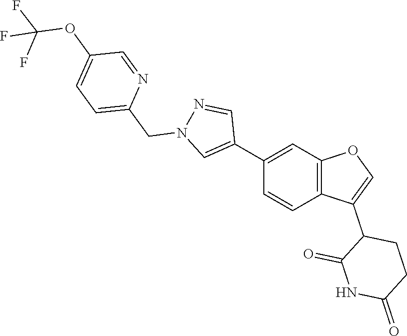 [SMILES: O=C1CCC(c2coc3cc(-c4cnn(Cc5ccc(OC(F)(F)F)cn5)c4)ccc23)C(=O)N1] |
| 362. | 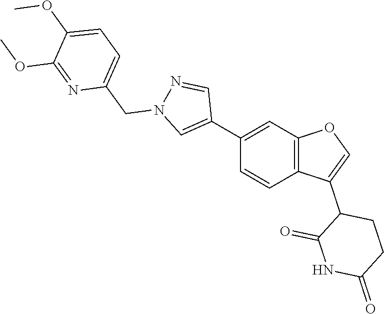 [SMILES: COc1ccc(Cn2cc(-c3ccc4c(C5CCC(=O)NC5=O)coc4c3)cn2)nc1OC] |
| 363. | 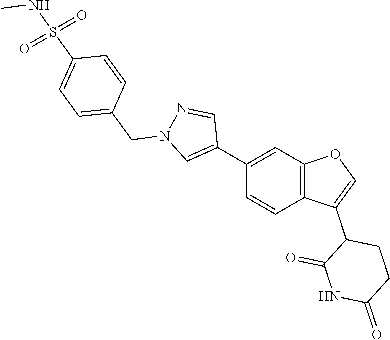 [SMILES: CNS(=O)(=O)c1ccc(Cn2cc(-c3ccc4c(C5CCC(=O)NC5=O)coc4c3)cn2)cc1] |
| 364. | 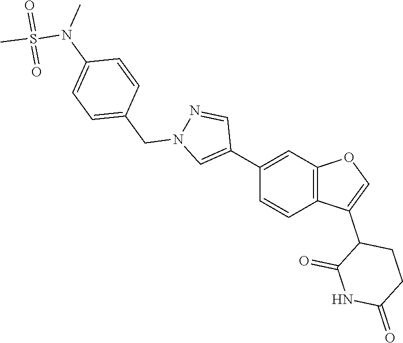 [SMILES: CN(c1ccc(Cn2cc(-c3ccc4c(C5CCC(=O)NC5=O)coc4c3)cn2)cc1)S(C)(=O)=O] |
| 365. | 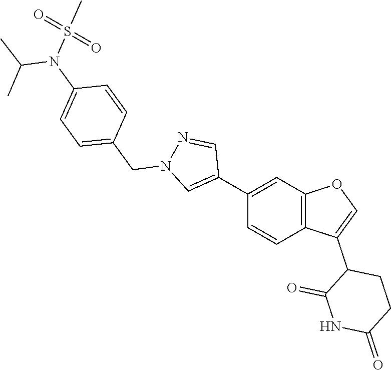 [SMILES: CC(C)N(c1ccc(Cn2cc(-c3ccc4c(C5CCC(=O)NC5=O)coc4c3)cn2)cc1)S(C)(=O)=O] |
| 366. | 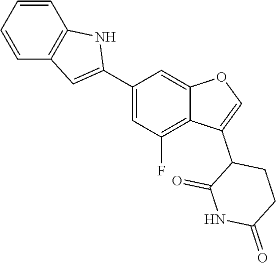 [SMILES: O=C1CCC(c2coc3cc(-c4cc5ccccc5[nH]4)cc(F)c23)C(=O)N1] |
| 367. | 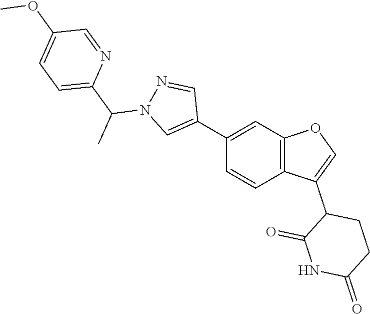 [SMILES: COc1ccc(C(C)n2cc(-c3ccc4c(C5CCC(=O)NC5=O)coc4c3)cn2)nc1] |
| 368. | 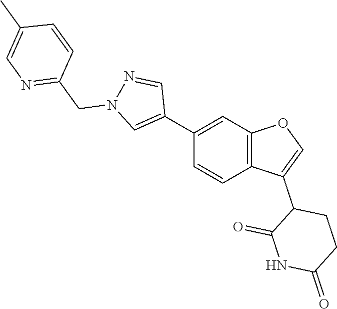 [SMILES: Cc1ccc(Cn2cc(-c3ccc4c(C5CCC(=O)NC5=O)coc4c3)cn2)nc1] |
| 369. | 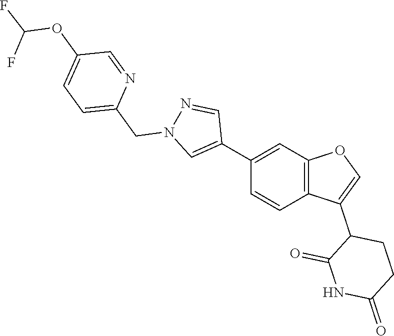 [SMILES: O=C1CCC(c2coc3cc(-c4cnn(Cc5ccc(OC(F)F)cn5)c4)ccc23)C(=O)N1] |
| 370. | 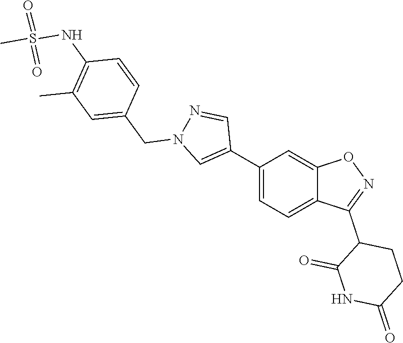 [SMILES: Cc1cc(Cn2cc(-c3ccc4c(C5CCC(=O)NC5=O)noc4c3)cn2)ccc1NS(C)(=O)=O] |
| 371. | 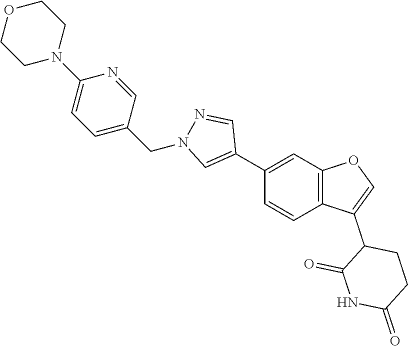 [SMILES: O=C1CCC(c2coc3cc(-c4cnn(Cc5ccc(N6CCOCC6)nc5)c4)ccc23)C(=O)N1] |
| 372. | 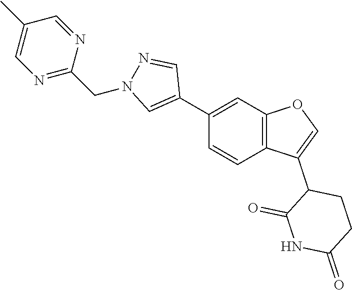 [SMILES: Cc1cnc(Cn2cc(-c3ccc4c(C5CCC(=O)NC5=O)coc4c3)cn2)nc1] |
| 373. | 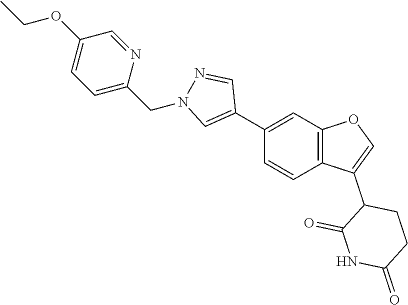 [SMILES: CCOc1ccc(Cn2cc(-c3ccc4c(C5CCC(=O)NC5=O)coc4c3)cn2)nc1] |
| 374. | 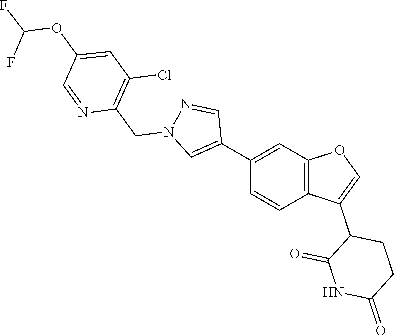 [SMILES: O=C1CCC(c2coc3cc(-c4cnn(Cc5ncc(OC(F)F)cc5Cl)c4)ccc23)C(=O)N1] |
| 375. | 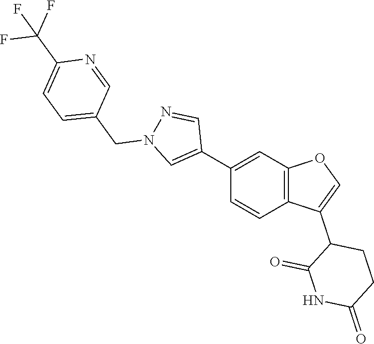 [SMILES: O=C1CCC(c2coc3cc(-c4cnn(Cc5ccc(C(F)(F)F)nc5)c4)ccc23)C(=O)N1] |
| 376. | 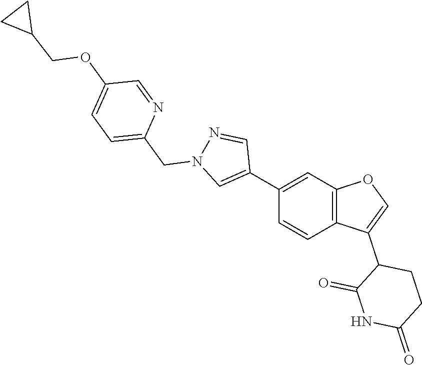 [SMILES: O=C1CCC(c2coc3cc(-c4cnn(Cc5ccc(OCC6CC6)cn5)c4)ccc23)C(=O)N1] |
| 377. | 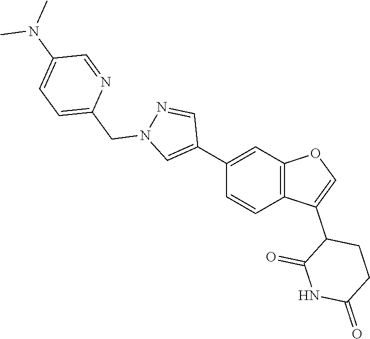 [SMILES: CN(C)c1ccc(Cn2cc(-c3ccc4c(C5CCC(=O)NC5=O)coc4c3)cn2)nc1] |
| 378. | 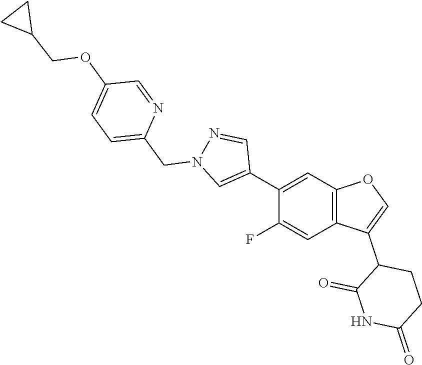 [SMILES: O=C1CCC(c2coc3cc(-c4cnn(Cc5ccc(OCC6CC6)cn5)c4)c(F)cc23)C(=O)N1] |
| 379. | 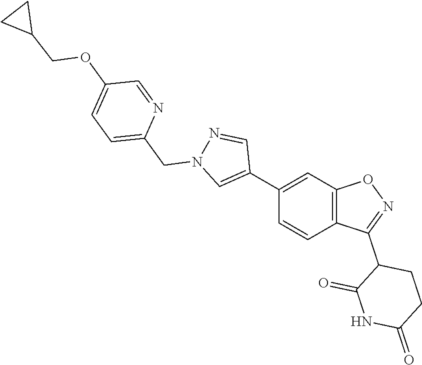 [SMILES: O=C1CCC(c2noc3cc(-c4cnn(Cc5ccc(OCC6CC6)cn5)c4)ccc23)C(=O)N1] |
| 380. | 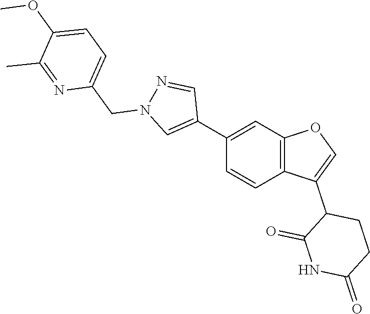 [SMILES: COc1ccc(Cn2cc(-c3ccc4c(C5CCC(=O)NC5=O)coc4c3)cn2)nc1C] |
| 381. | 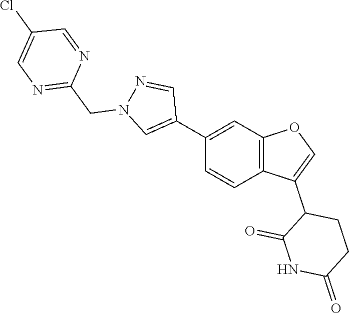 [SMILES: O=C1CCC(c2coc3cc(-c4cnn(Cc5ncc(Cl)cn5)c4)ccc23)C(=O)N1] |
| 382. | 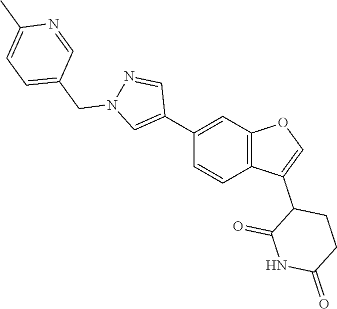 [SMILES: Cc1ccc(Cn2cc(-c3ccc4c(C5CCC(=O)NC5=O)coc4c3)cn2)cn1] |
| 383. | 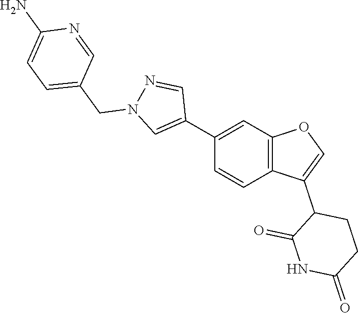 [SMILES: Nc1ccc(Cn2cc(-c3ccc4c(C5CCC(=O)NC5=O)coc4c3)cn2)cn1] |
| 384. | 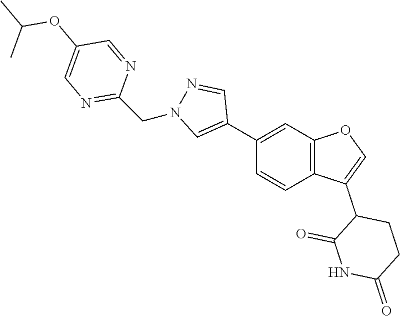 [SMILES: CC(C)Oc1cnc(Cn2cc(-c3ccc4c(C5CCC(=O)NC5=O)coc4c3)cn2)nc1] |
| 385. | 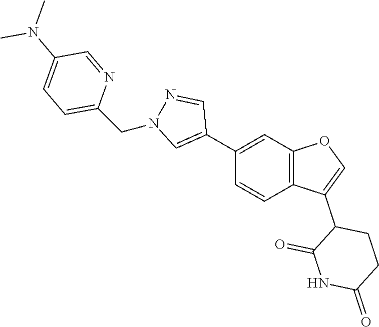 [SMILES: CN(C)c1ccc(Cn2cc(-c3ccc4c(C5CCC(=O)NC5=O)coc4c3)cn2)nc1] |
| 386. | 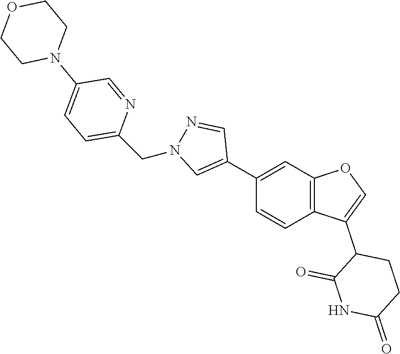 [SMILES: O=C1CCC(c2coc3cc(-c4cnn(Cc5ccc(N6CCOCC6)cn5)c4)ccc23)C(=O)N1] |
| 387. | 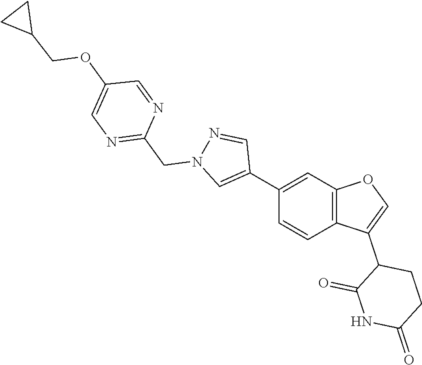 [SMILES: O=C1CCC(c2coc3cc(-c4cnn(Cc5ncc(OCC6CC6)cn5)c4)ccc23)C(=O)N1] |
| 388. | 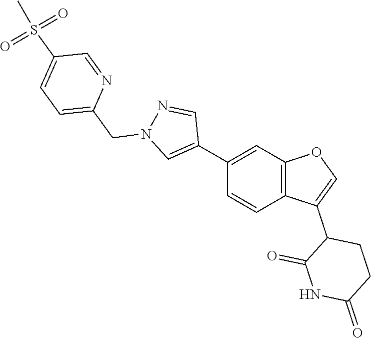 [SMILES: CS(=O)(=O)c1ccc(Cn2cc(-c3ccc4c(C5CCC(=O)NC5=O)coc4c3)cn2)nc1] |
| 389. | 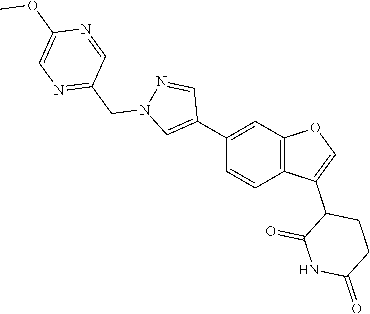 [SMILES: COc1cnc(Cn2cc(-c3ccc4c(C5CCC(=O)NC5=O)coc4c3)cn2)cn1] |
| 390. | 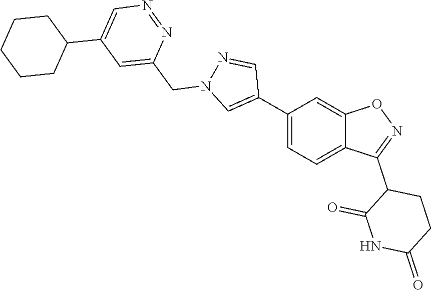 [SMILES: O=C1CCC(c2noc3cc(-c4cnn(Cc5cc(C6CCCCC6)cnn5)c4)ccc23)C(=O)N1] |
| 391. | 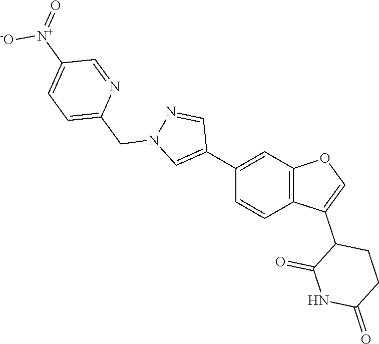 |
| 392. | 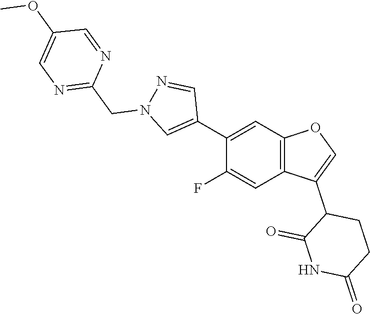 [SMILES: COc1cnc(Cn2cc(-c3cc4occ(C5CCC(=O)NC5=O)c4cc3F)cn2)nc1] |
| 393. | 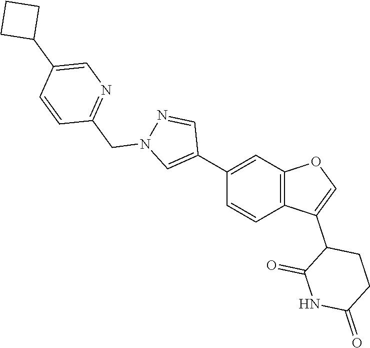 [SMILES: O=C1CCC(c2coc3cc(-c4cnn(Cc5ccc(C6CCC6)cn5)c4)ccc23)C(=O)N1] |
| 394. | 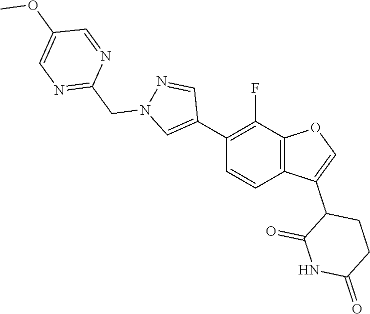 [SMILES: COc1cnc(Cn2cc(-c3ccc4c(C5CCC(=O)NC5=O)coc4c3F)cn2)nc1] |
| 395. | 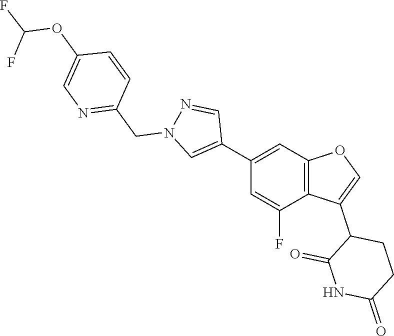 [SMILES: O=C1CCC(c2coc3cc(-c4cnn(Cc5ccc(OC(F)F)cn5)c4)cc(F)c23)C(=O)N1] |
| 396. | 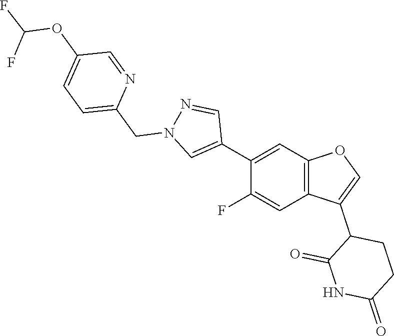 [SMILES: O=C1CCC(c2coc3cc(-c4cnn(Cc5ccc(OC(F)F)cn5)c4)c(F)cc23)C(=O)N1] |
| 397. | 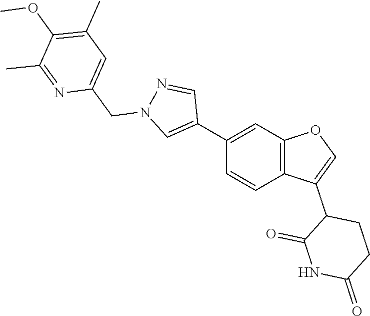 [SMILES: COc1c(C)cc(Cn2cc(-c3ccc4c(C5CCC(=O)NC5=O)coc4c3)cn2)nc1C] |
| 398. | 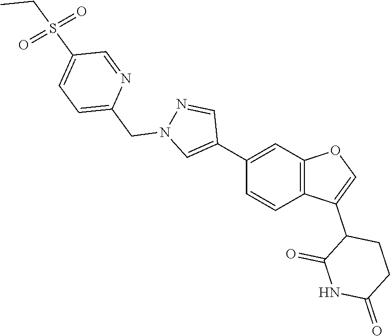 [SMILES: CCS(=O)(=O)c1ccc(Cn2cc(-c3ccc4c(C5CCC(=O)NC5=O)coc4c3)cn2)nc1] |
| 399. | 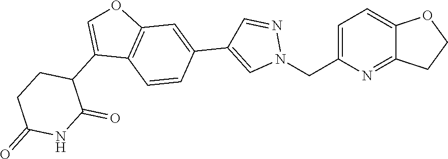 [SMILES: O=C1CCC(c2coc3cc(-c4cnn(Cc5ccc6c(n5)CCO6)c4)ccc23)C(=O)N1] |
| 400. | 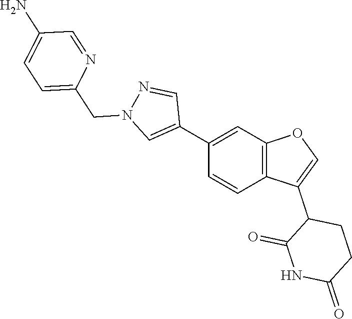 [SMILES: Nc1ccc(Cn2cc(-c3ccc4c(C5CCC(=O)NC5=O)coc4c3)cn2)nc1] |
| 401. | 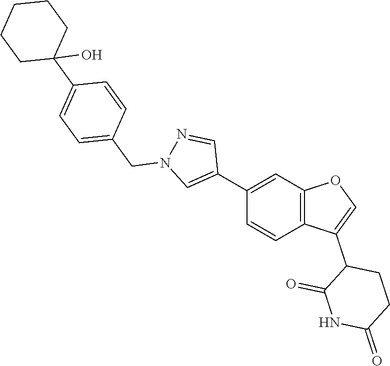 [SMILES: O=C1CCC(c2coc3cc(-c4cnn(Cc5ccc(C6(O)CCCCC6)cc5)c4)ccc23)C(=O)N1] |
| 402. | 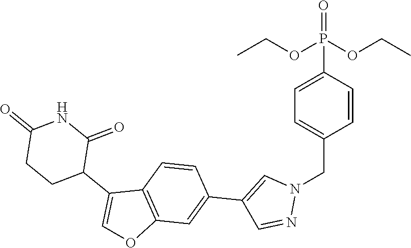 [SMILES: CCOP(=O)(OCC)c1ccc(Cn2cc(-c3ccc4c(C5CCC(=O)NC5=O)coc4c3)cn2)cc1] |
| 403. | 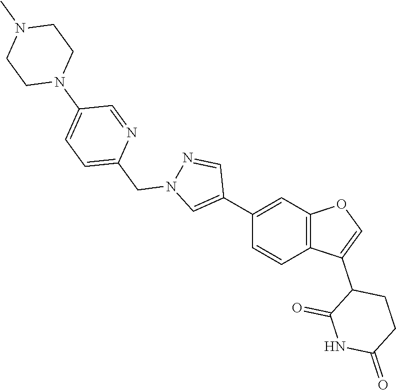 [SMILES: CN1CCN(c2ccc(Cn3cc(-c4ccc5c(C6CCC(=O)NC6=O)coc5c4)cn3)nc2)CC1] |
| 404. | 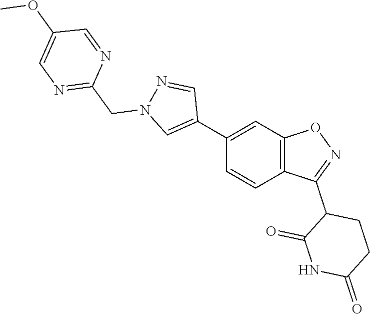 [SMILES: COc1cnc(Cn2cc(-c3ccc4c(C5CCC(=O)NC5=O)noc4c3)cn2)nc1] |
| 405. | 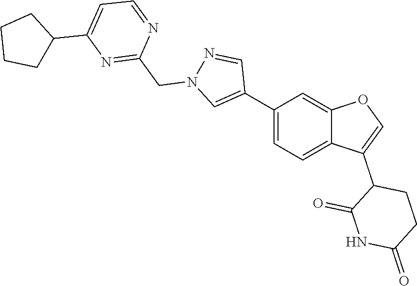 [SMILES: O=C1CCC(c2coc3cc(-c4cnn(Cc5nccc(C6CCCC6)n5)c4)ccc23)C(=O)N1] |
| 406. | 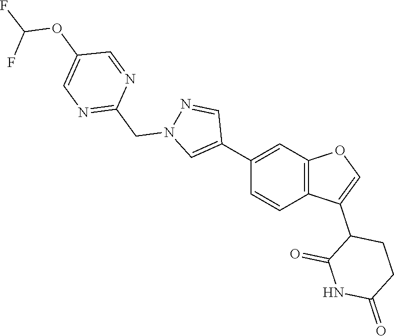 [SMILES: O=C1CCC(c2coc3cc(-c4cnn(Cc5ncc(OC(F)F)cn5)c4)ccc23)C(=O)N1] |
| 407. | 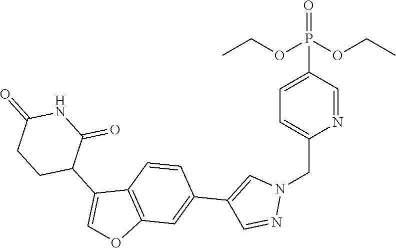 [SMILES: CCOP(=O)(OCC)c1ccc(Cn2cc(-c3ccc4c(C5CCC(=O)NC5=O)coc4c3)cn2)nc1] |
| 408. | 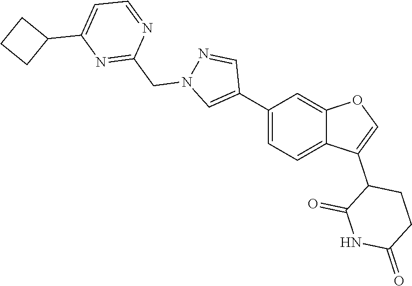 [SMILES: O=C1CCC(c2coc3cc(-c4cnn(Cc5nccc(C6CCC6)n5)c4)ccc23)C(=O)N1] |
| 409. | 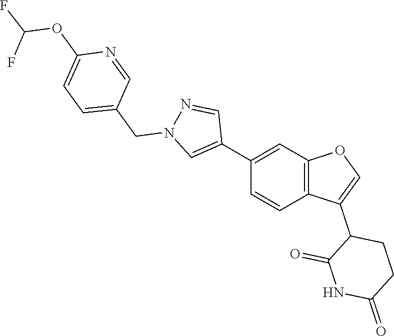 [SMILES: O=C1CCC(c2coc3cc(-c4cnn(Cc5ccc(OC(F)F)nc5)c4)ccc23)C(=O)N1] |
| 410. | 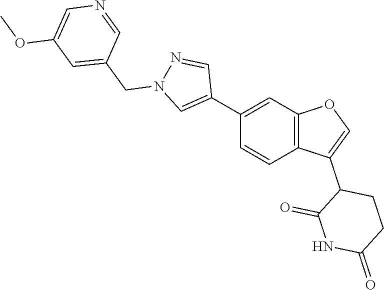 [SMILES: COc1cncc(Cn2cc(-c3ccc4c(C5CCC(=O)NC5=O)coc4c3)cn2)c1] |
| 411. | 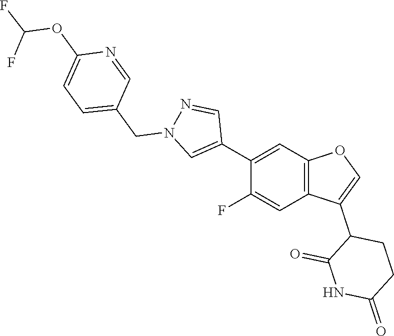 [SMILES: O=C1CCC(c2coc3cc(-c4cnn(Cc5ccc(OC(F)F)nc5)c4)c(F)cc23)C(=O)N1] |
| 412. | 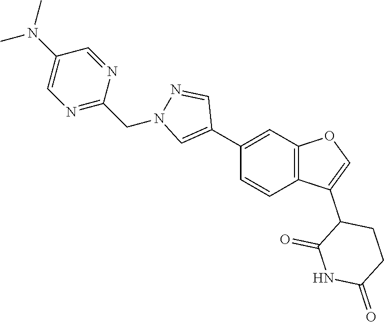 [SMILES: CN(C)c1cnc(Cn2cc(-c3ccc4c(C5CCC(=O)NC5=O)coc4c3)cn2)nc1] |
| 413. | 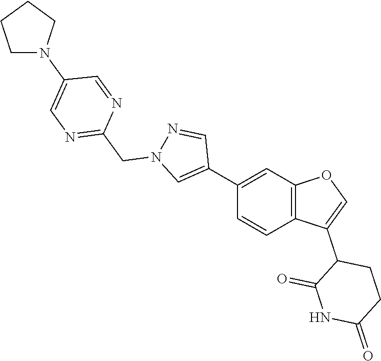 [SMILES: O=C1CCC(c2coc3cc(-c4cnn(Cc5ncc(N6CCCC6)cn5)c4)ccc23)C(=O)N1] |
| 414. | 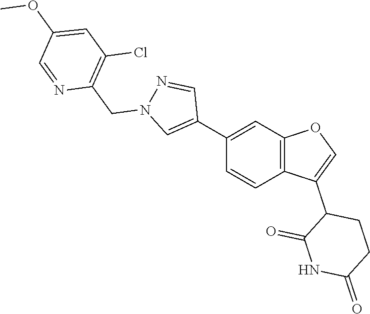 [SMILES: COc1cnc(Cn2cc(-c3ccc4c(C5CCC(=O)NC5=O)coc4c3)cn2)c(Cl)c1] |
| 415. | 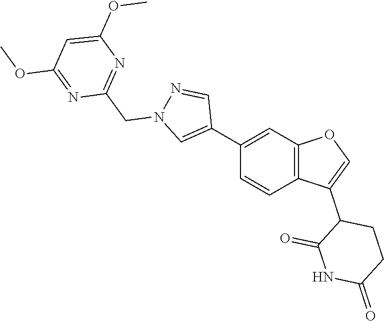 [SMILES: COc1cc(OC)nc(Cn2cc(-c3ccc4c(C5CCC(=O)NC5=O)coc4c3)cn2)n1] |
| 416. | 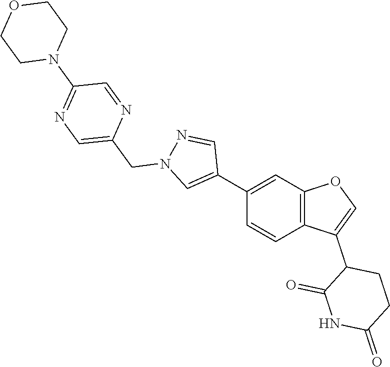 [SMILES: O=C1CCC(c2coc3cc(-c4cnn(Cc5cnc(N6CCOCC6)cn5)c4)ccc23)C(=O)N1] |
| 417. | 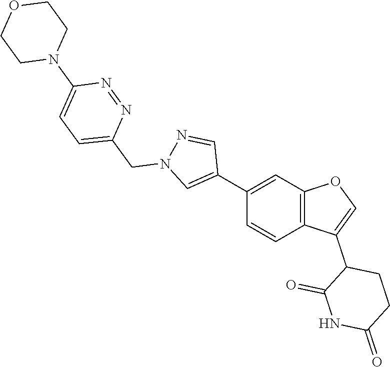 [SMILES: O=C1CCC(c2coc3cc(-c4cnn(Cc5ccc(N6CCOCC6)nn5)c4)ccc23)C(=O)N1] |
| 418. | 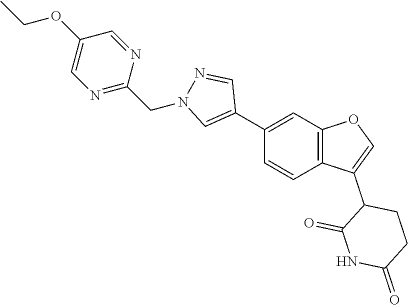 [SMILES: CCOc1cnc(Cn2cc(-c3ccc4c(C5CCC(=O)NC5=O)coc4c3)cn2)nc1] |
| 419. | 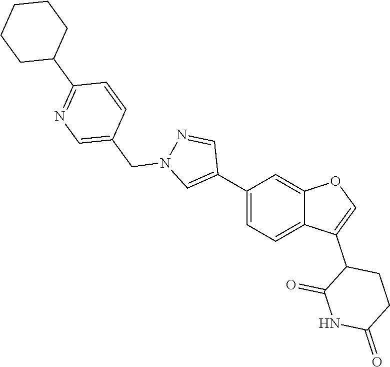 [SMILES: O=C1CCC(c2coc3cc(-c4cnn(Cc5ccc(C6CCCCC6)nc5)c4)ccc23)C(=O)N1] |
| 420. | 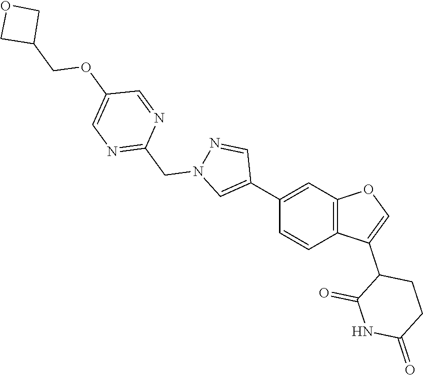 [SMILES: O=C1CCC(c2coc3cc(-c4cnn(Cc5ncc(OCC6COC6)cn5)c4)ccc23)C(=O)N1] |
| 421. | 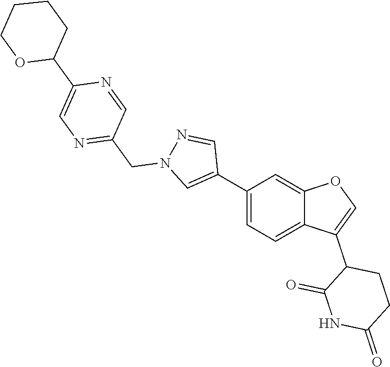 [SMILES: O=C1CCC(c2coc3cc(-c4cnn(Cc5cnc(C6CCCCO6)cn5)c4)ccc23)C(=O)N1] |
| 422. | 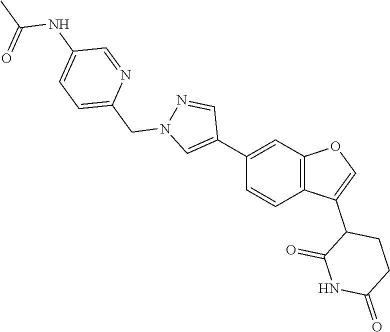 [SMILES: CC(=O)Nc1ccc(Cn2cc(-c3ccc4c(C5CCC(=O)NC5=O)coc4c3)cn2)nc1] |
| 423. | 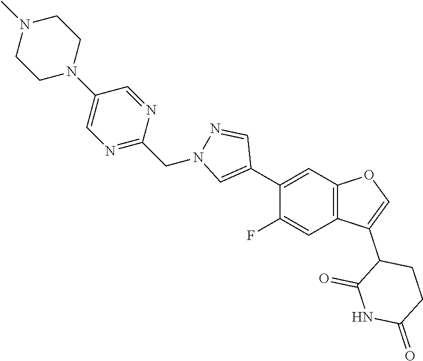 [SMILES: CN1CCN(c2cnc(Cn3cc(-c4cc5occ(C6CCC(=O)NC6=O)c5cc4F)cn3)nc2)CC1] |
| 424. | 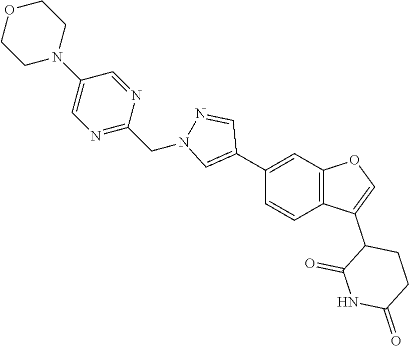 [SMILES: O=C1CCC(c2coc3cc(-c4cnn(Cc5ncc(N6CCOCC6)cn5)c4)ccc23)C(=O)N1] |
| 425. | 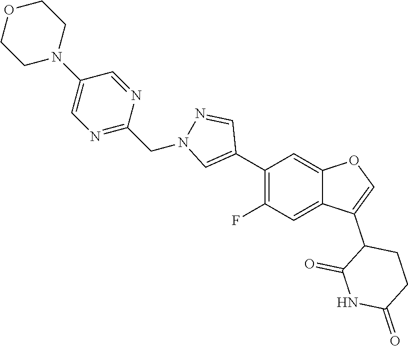 [SMILES: O=C1CCC(c2coc3cc(-c4cnn(Cc5ncc(N6CCOCC6)cn5)c4)c(F)cc23)C(=O)N1] |
| 426. | 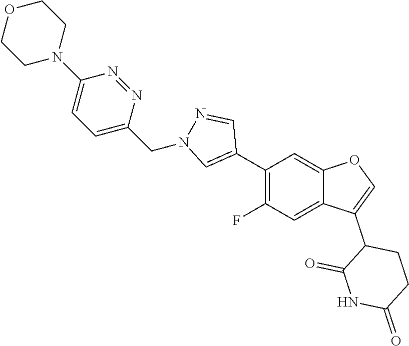 [SMILES: O=C1CCC(c2coc3cc(-c4cnn(Cc5ccc(N6CCOCC6)nn5)c4)c(F)cc23)C(=O)N1] |
| 427. | 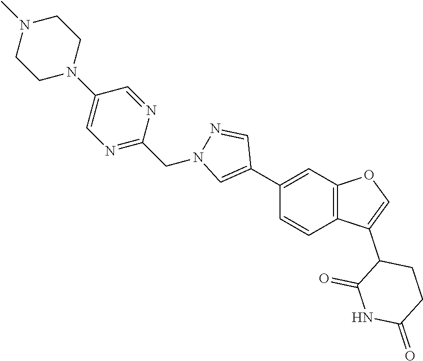 [SMILES: CN1CCN(c2cnc(Cn3cc(-c4ccc5c(C6CCC(=O)NC6=O)coc5c4)cn3)nc2)CC1] |
| 428. | 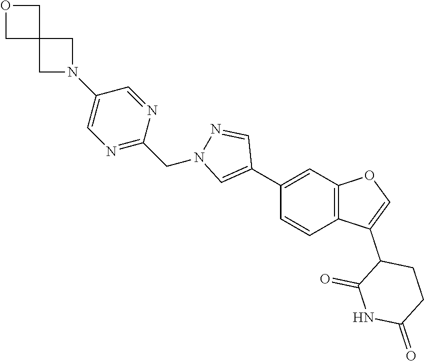 [SMILES: O=C1CCC(c2coc3cc(-c4cnn(Cc5ncc(N6CC7(COC7)C6)cn5)c4)ccc23)C(=O)N1] |
| 429. | 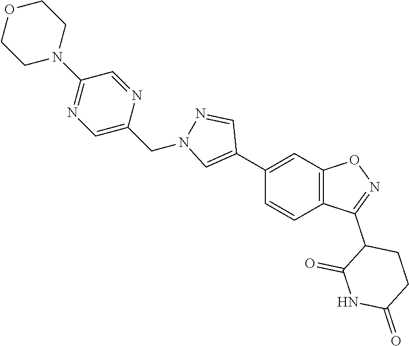 [SMILES: O=C1CCC(c2noc3cc(-c4cnn(Cc5cnc(N6CCOCC6)cn5)c4)ccc23)C(=O)N1] |
| 430. | 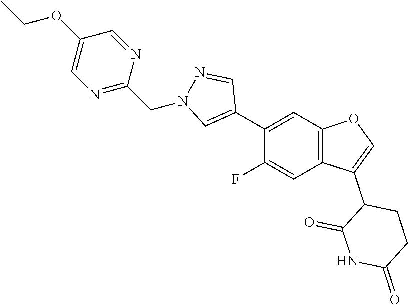 [SMILES: CCOc1cnc(Cn2cc(-c3cc4occ(C5CCC(=O)NC5=O)c4cc3F)cn2)nc1] |
| 431. | 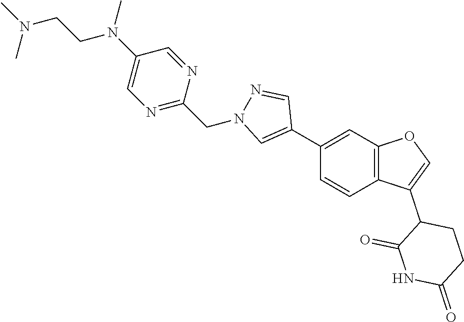 [SMILES: CN(C)CCN(C)c1cnc(Cn2cc(-c3ccc4c(C5CCC(=O)NC5=O)coc4c3)cn2)nc1] |
| 432. | 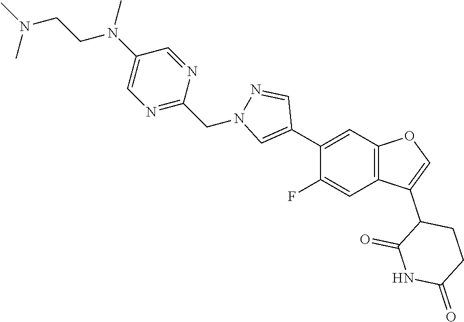 [SMILES: CN(C)CCN(C)c1cnc(Cn2cc(-c3cc4occ(C5CCC(=O)NC5=O)c4cc3F)cn2)nc1] |
| 433. | 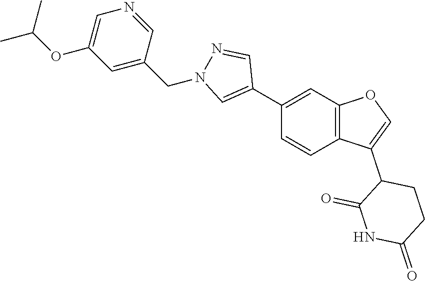 [SMILES: CC(C)Oc1cncc(Cn2cc(-c3ccc4c(C5CCC(=O)NC5=O)coc4c3)cn2)c1] |
| 434. | 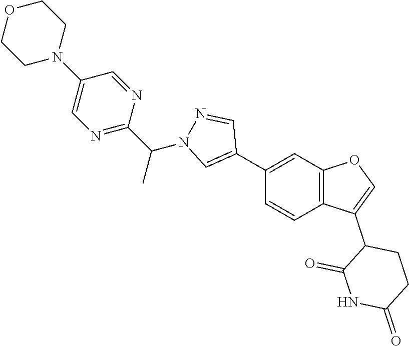 [SMILES: CC(c1ncc(N2CCOCC2)cn1)n1cc(-c2ccc3c(C4CCC(=O)NC4=O)coc3c2)cn1] |
| 435. | 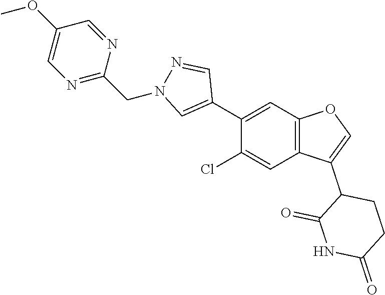 [SMILES: COc1cnc(Cn2cc(-c3cc4occ(C5CCC(=O)NC5=O)c4cc3Cl)cn2)nc1] |
| 436. | 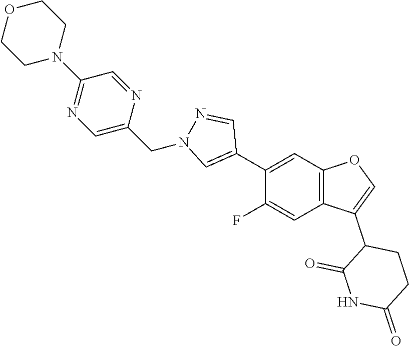 [SMILES: O=C1CCC(c2coc3cc(-c4cnn(Cc5cnc(N6CCOCC6)cn5)c4)c(F)cc23)C(=O)N1] |
| 437. | 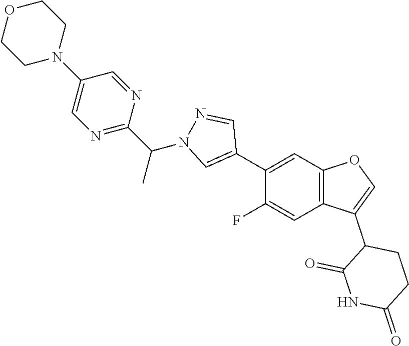 [SMILES: CC(c1ncc(N2CCOCC2)cn1)n1cc(-c2cc3occ(C4CCC(=O)NC4=O)c3cc2F)cn1] |
| 438. | 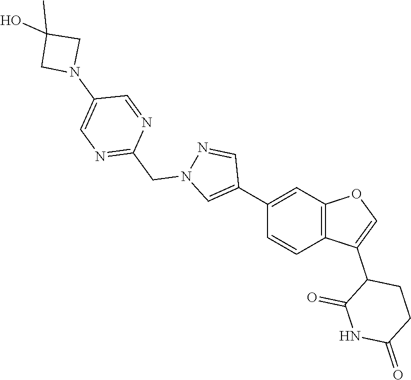 [SMILES: CC1(O)CN(c2cnc(Cn3cc(-c4ccc5c(C6CCC(=O)NC6=O)coc5c4)cn3)nc2)C1] |
| 439. | 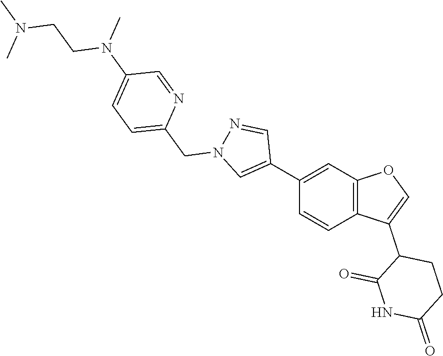 [SMILES: CN(C)CCN(C)c1ccc(Cn2cc(-c3ccc4c(C5CCC(=O)NC5=O)coc4c3)cn2)nc1] |
| 440. | 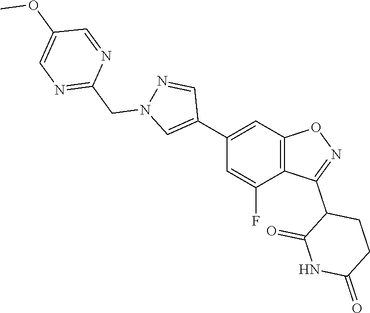 [SMILES: COc1cnc(Cn2cc(-c3cc(F)c4c(C5CCC(=O)NC5=O)noc4c3)cn2)nc1] |
| 441. | 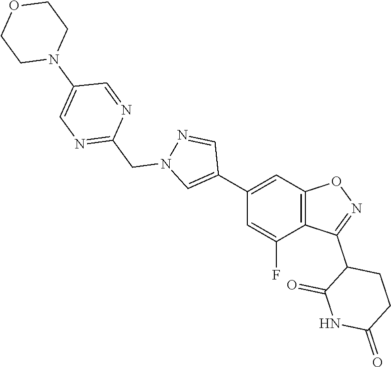 [SMILES: O=C1CCC(c2noc3cc(-c4cnn(Cc5ncc(N6CCOCC6)cn5)c4)cc(F)c23)C(=O)N1] |
| 442. | 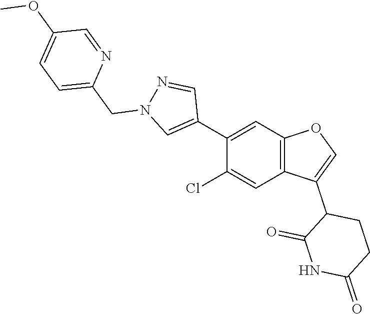 [SMILES: COc1ccc(Cn2cc(-c3cc4occ(C5CCC(=O)NC5=O)c4cc3Cl)cn2)nc1] |
| 443. | 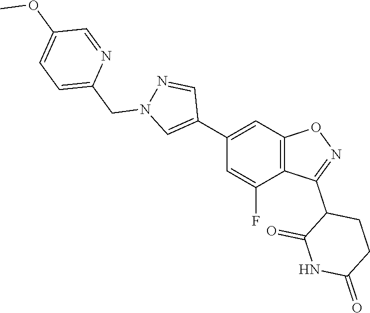 [SMILES: COc1ccc(Cn2cc(-c3cc(F)c4c(C5CCC(=O)NC5=O)noc4c3)cn2)nc1] |
| 444. | 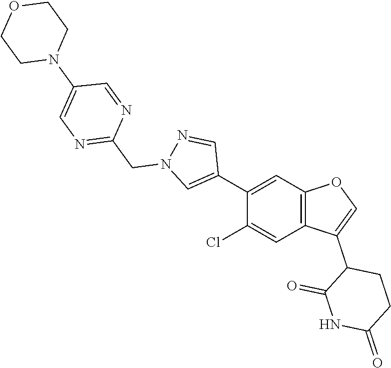 [SMILES: O=C1CCC(c2coc3cc(-c4cnn(Cc5ncc(N6CCOCC6)cn5)c4)c(Cl)cc23)C(=O)N1] |
| 445. | 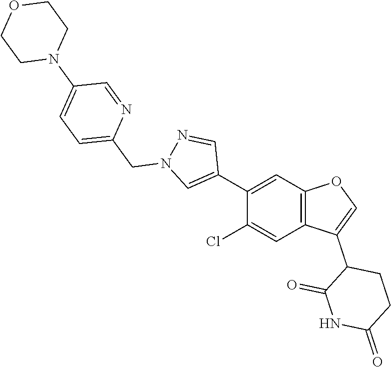 [SMILES: O=C1CCC(c2coc3cc(-c4cnn(Cc5ccc(N6CCOCC6)cn5)c4)c(Cl)cc23)C(=O)N1] |
| 446. | 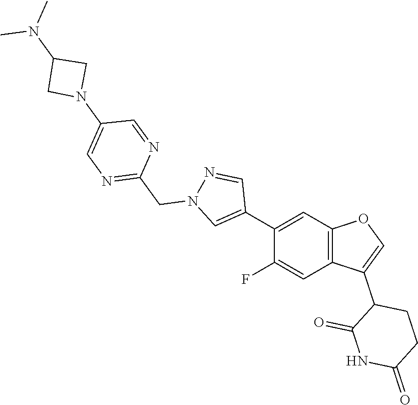 [SMILES: CN(C)C1CN(c2cnc(Cn3cc(-c4cc5occ(C6CCC(=O)NC6=O)c5cc4F)cn3)nc2)C1] |
| 447. | 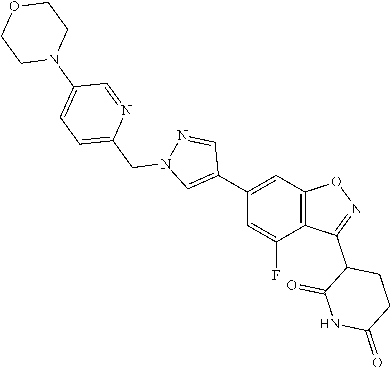 [SMILES: O=C1CCC(c2noc3cc(-c4cnn(Cc5ccc(N6CCOCC6)cn5)c4)cc(F)c23)C(=O)N1] |
| 448. | 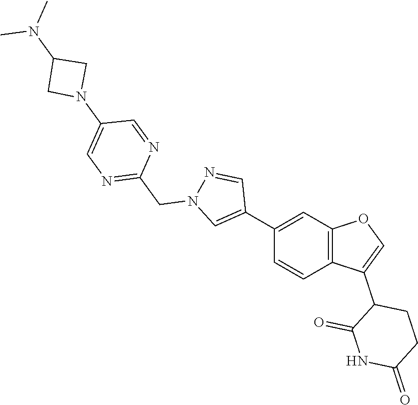 [SMILES: CN(C)C1CN(c2cnc(Cn3cc(-c4ccc5c(C6CCC(=O)NC6=O)coc5c4)cn3)nc2)C1] |
| 449. | 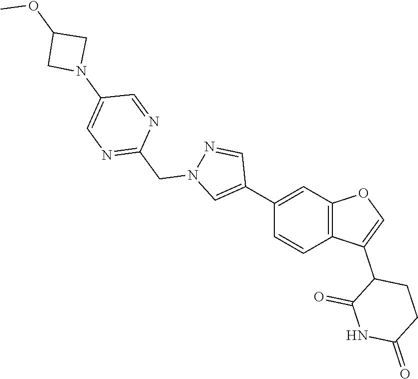 [SMILES: COC1CN(c2cnc(Cn3cc(-c4ccc5c(C6CCC(=O)NC6=O)coc5c4)cn3)nc2)C1] |
| 450. | 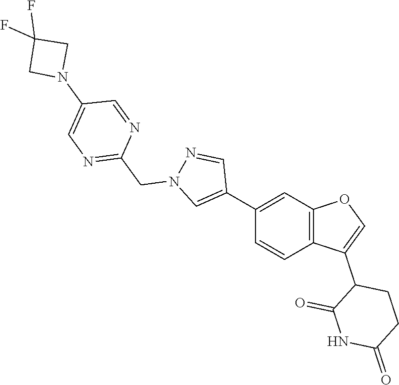 [SMILES: O=C1CCC(c2coc3cc(-c4cnn(Cc5ncc(N6CC(F)(F)C6)cn5)c4)ccc23)C(=O)N1] |
| 451. | 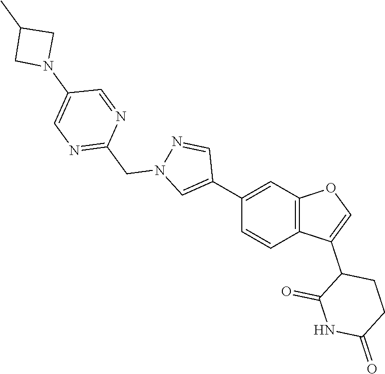 [SMILES: CC1CN(c2cnc(Cn3cc(-c4ccc5c(C6CCC(=O)NC6=O)coc5c4)cn3)nc2)C1] |
| 452. | 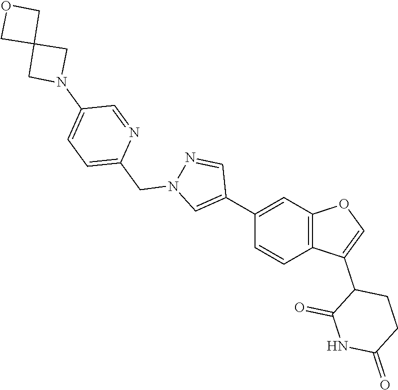 [SMILES: O=C1CCC(c2coc3cc(-c4cnn(Cc5ccc(N6CC7(COC7)C6)cn5)c4)ccc23)C(=O)N1] |
| 453. | 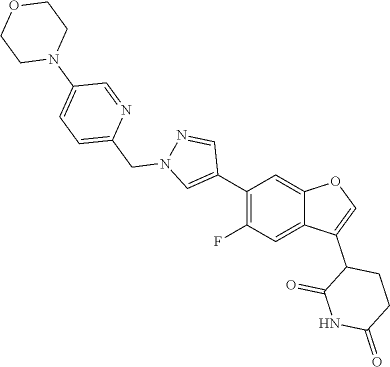 [SMILES: O=C1CCC(c2coc3cc(-c4cnn(Cc5ccc(N6CCOCC6)cn5)c4)c(F)cc23)C(=O)N1] |
| 454. | 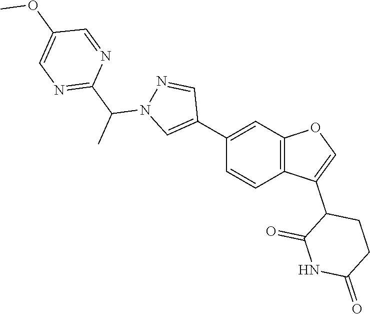 [SMILES: COc1cnc(C(C)n2cc(-c3ccc4c(C5CCC(=O)NC5=O)coc4c3)cn2)nc1] |
| 455. | 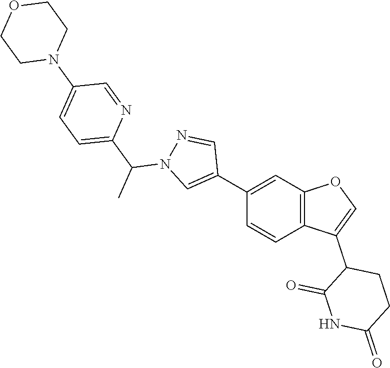 [SMILES: CC(c1ccc(N2CCOCC2)cn1)n1cc(-c2ccc3c(C4CCC(=O)NC4=O)coc3c2)cn1] |
| 456. | 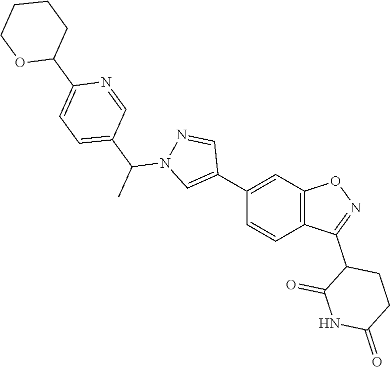 [SMILES: CC(c1ccc(C2CCCCO2)nc1)n1cc(-c2ccc3c(C4CCC(=O)NC4=O)noc3c2)cn1] |
| 457. | 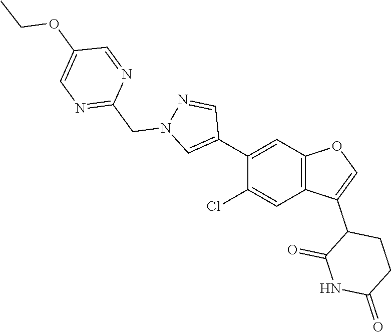 [SMILES: CCOc1cnc(Cn2cc(-c3cc4occ(C5CCC(=O)NC5=O)c4cc3Cl)cn2)nc1] |
| 458. | 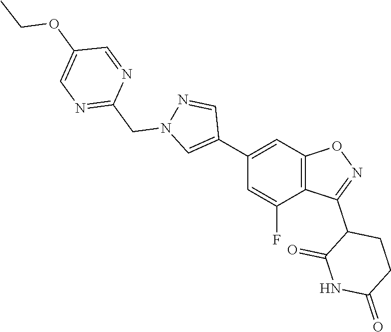 [SMILES: CCOc1cnc(Cn2cc(-c3cc(F)c4c(C5CCC(=O)NC5=O)noc4c3)cn2)nc1] |
| 459. | 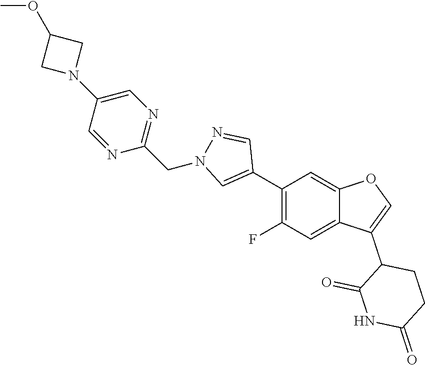 [SMILES: COC1CN(c2cnc(Cn3cc(-c4cc5occ(C6CCC(=O)NC6=O)c5cc4F)cn3)nc2)C1] |
| 460. | 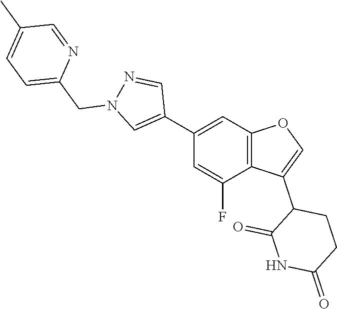 [SMILES: Cc1ccc(Cn2cc(-c3cc(F)c4c(C5CCC(=O)NC5=O)coc4c3)cn2)nc1] |
| 461. | 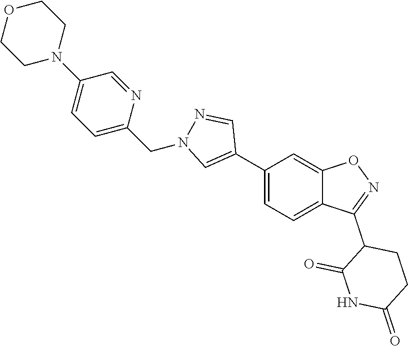 [SMILES: O=C1CCC(c2noc3cc(-c4cnn(Cc5ccc(N6CCOCC6)cn5)c4)ccc23)C(=O)N1] |
| 462. | 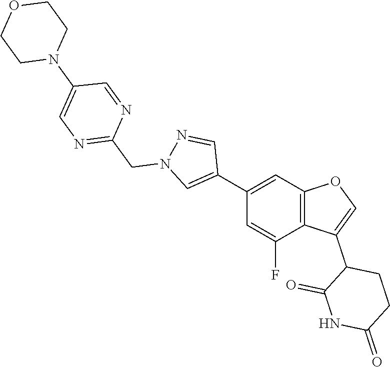 [SMILES: O=C1CCC(c2coc3cc(-c4cnn(Cc5ncc(N6CCOCC6)cn5)c4)cc(F)c23)C(=O)N1] |
| 463. | 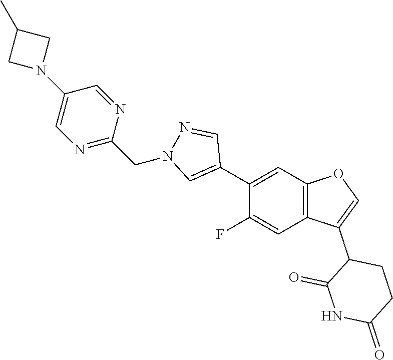 [SMILES: CC1CN(c2cnc(Cn3cc(-c4cc5occ(C6CCC(=O)NC6=O)c5cc4F)cn3)nc2)C1] |
| 464. | 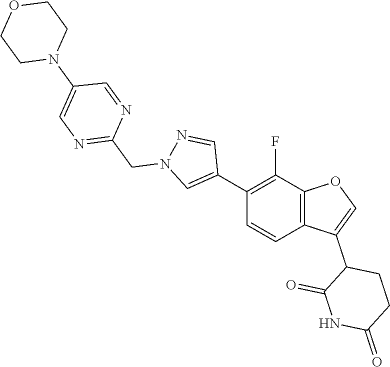 [SMILES: O=C1CCC(c2coc3c(F)c(-c4cnn(Cc5ncc(N6CCOCC6)cn5)c4)ccc23)C(=O)N1] |
| 465. | 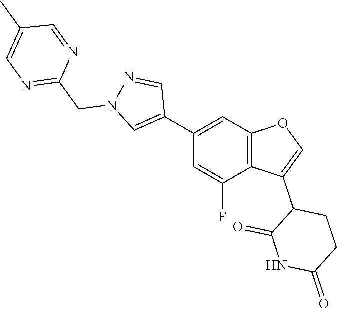 [SMILES: Cc1cnc(Cn2cc(-c3cc(F)c4c(C5CCC(=O)NC5=O)coc4c3)cn2)nc1] |
| 466. | 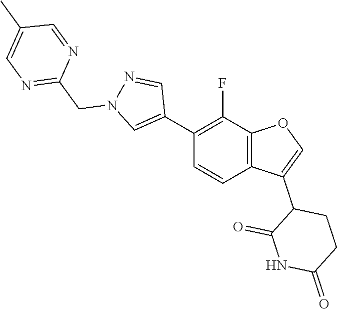 [SMILES: Cc1cnc(Cn2cc(-c3ccc4c(C5CCC(=O)NC5=O)coc4c3F)cn2)nc1] |
| 467. | 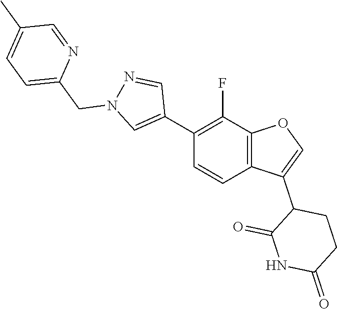 [SMILES: Cc1ccc(Cn2cc(-c3ccc4c(C5CCC(=O)NC5=O)coc4c3F)cn2)nc1] |
| 468. | 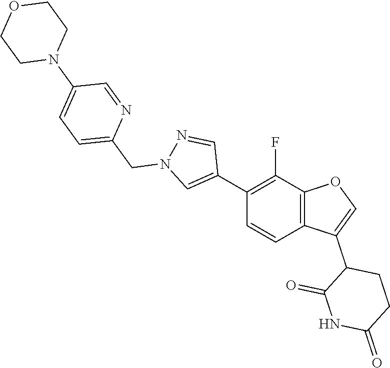 [SMILES: O=C1CCC(c2coc3c(F)c(-c4cnn(Cc5ccc(N6CCOCC6)cn5)c4)ccc23)C(=O)N1] |
| 469. | 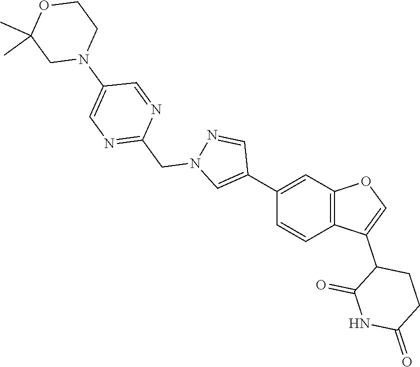 [SMILES: CC1(C)CN(c2cnc(Cn3cc(-c4ccc5c(C6CCC(=O)NC6=O)coc5c4)cn3)nc2)CCO1] |
| 470. | 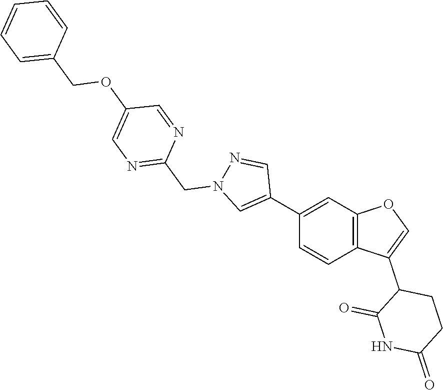 [SMILES: O=C1CCC(c2coc3cc(-c4cnn(Cc5ncc(OCc6ccccc6)cn5)c4)ccc23)C(=O)N1] |
| 471. | 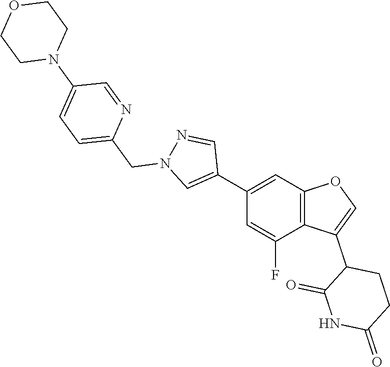 [SMILES: O=C1CCC(c2coc3cc(-c4cnn(Cc5ccc(N6CCOCC6)cn5)c4)cc(F)c23)C(=O)N1] |
| 472. | 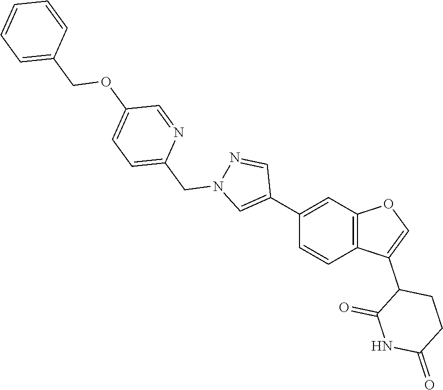 [SMILES: O=C1CCC(c2coc3cc(-c4cnn(Cc5ccc(OCc6ccccc6)cn5)c4)ccc23)C(=O)N1] |
| 473. | 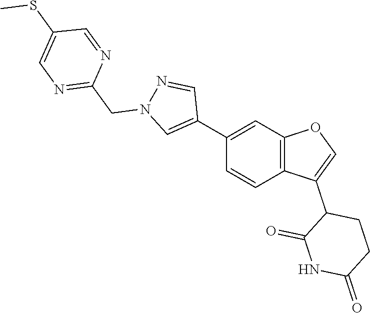 [SMILES: CSc1cnc(Cn2cc(-c3ccc4c(C5CCC(=O)NC5=O)coc4c3)cn2)nc1] |
| 474. | 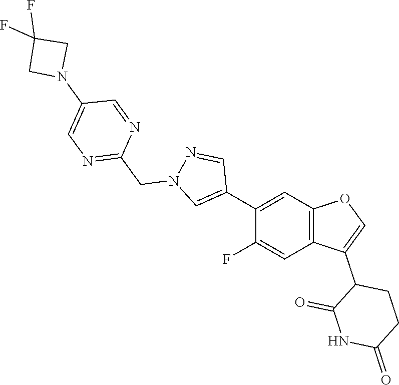 [SMILES: O=C1CCC(c2coc3cc(-c4cnn(Cc5ncc(N6CC(F)(F)C6)cn5)c4)c(F)cc23)C(=O)N1] |
| 475. | 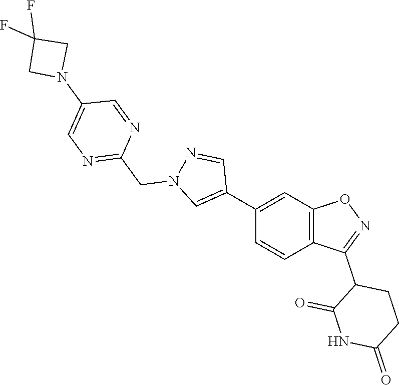 [SMILES: O=C1CCC(c2noc3cc(-c4cnn(Cc5ncc(N6CC(F)(F)C6)cn5)c4)ccc23)C(=O)N1] |
| 476. | 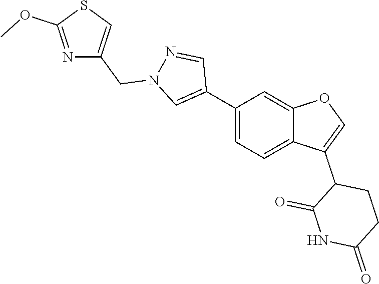 [SMILES: COc1nc(Cn2cc(-c3ccc4c(C5CCC(=O)NC5=O)coc4c3)cn2)cs1] |
| 477. | 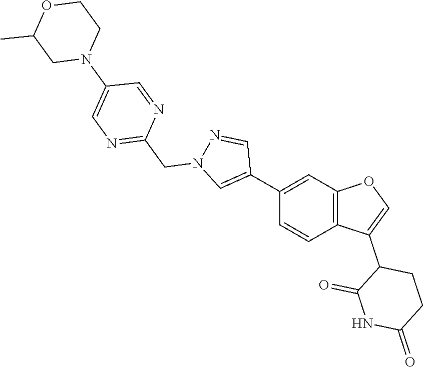 [SMILES: CC1CN(c2cnc(Cn3cc(-c4ccc5c(C6CCC(=O)NC6=O)coc5c4)cn3)nc2)CCO1] |
| 478. | 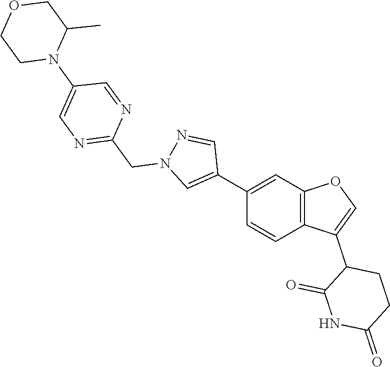 [SMILES: CC1COCCN1c1cnc(Cn2cc(-c3ccc4c(C5CCC(=O)NC5=O)coc4c3)cn2)nc1] |
| 479. | 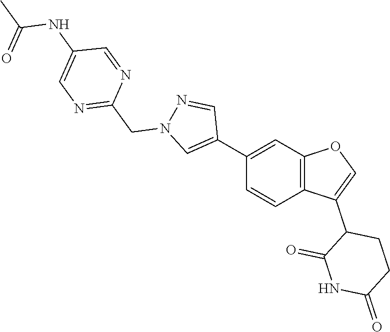 [SMILES: CC(=O)Nc1cnc(Cn2cc(-c3ccc4c(C5CCC(=O)NC5=O)coc4c3)cn2)nc1] |
| 480. | 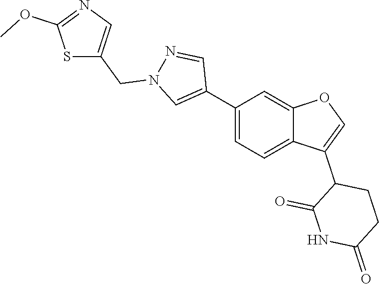 [SMILES: COc1ncc(Cn2cc(-c3ccc4c(C5CCC(=O)NC5=O)coc4c3)cn2)s1] |
| 481. | 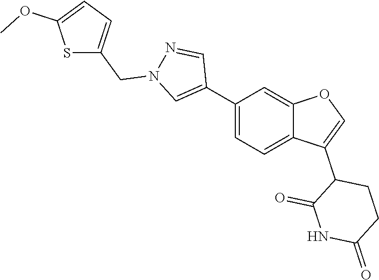 [SMILES: COc1ccc(Cn2cc(-c3ccc4c(C5CCC(=O)NC5=O)coc4c3)cn2)s1] |
| 482. | 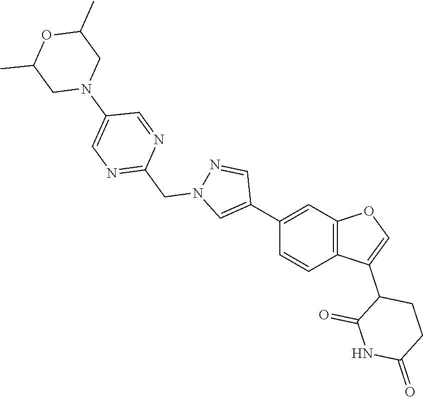 [SMILES: CC1CN(c2cnc(Cn3cc(-c4ccc5c(C6CCC(=O)NC6=O)coc5c4)cn3)nc2)CC(C)O1] |
| 483. | 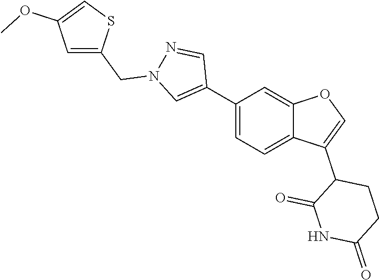 [SMILES: COc1csc(Cn2cc(-c3ccc4c(C5CCC(=O)NC5=O)coc4c3)cn2)c1] |
| 484. | 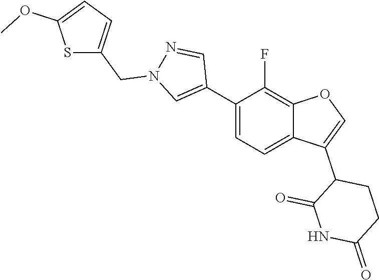 [SMILES: COc1ccc(Cn2cc(-c3ccc4c(C5CCC(=O)NC5=O)coc4c3F)cn2)s1] |
| 485. | 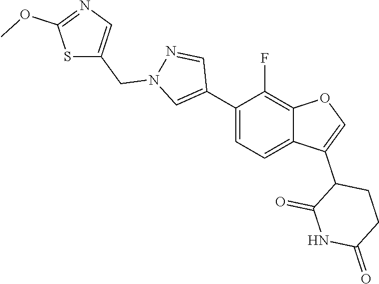 [SMILES: COc1ncc(Cn2cc(-c3ccc4c(C5CCC(=O)NC5=O)coc4c3F)cn2)s1] |
| 486. | 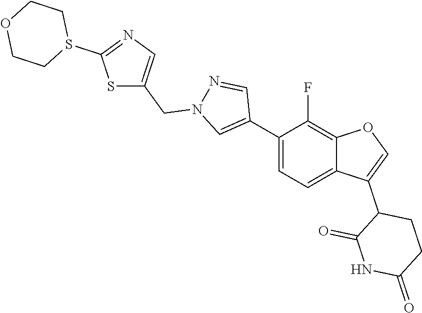 |
| 487. | 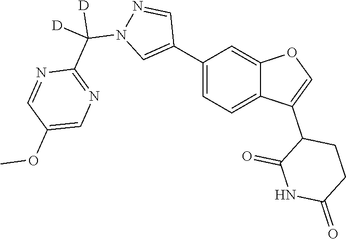 [SMILES: [2H]C([2H])(c1ncc(OC)cn1)n1cc(-c2ccc3c(C4CCC(=O)NC4=O)coc3c2)cn1] |
| 488. | 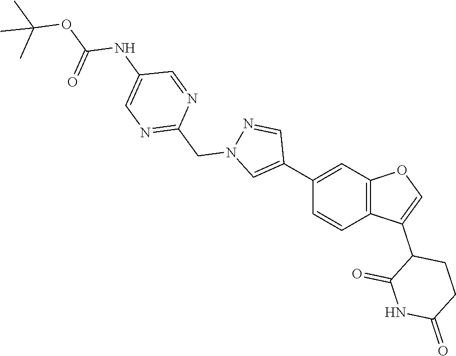 [SMILES: CC(C)(C)OC(=O)Nc1cnc(Cn2cc(-c3ccc4c(C5CCC(=O)NC5=O)coc4c3)cn2)nc1] |
| 489. | 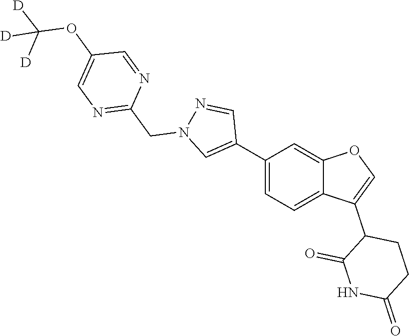 [SMILES: [2H]C([2H])([2H])Oc1cnc(Cn2cc(-c3ccc4c(C5CCC(=O)NC5=O)coc4c3)cn2)nc1] |
| 490. | 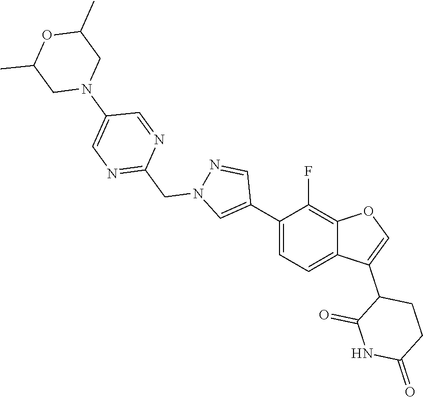 [SMILES: CC1CN(c2cnc(Cn3cc(-c4ccc5c(C6CCC(=O)NC6=O)coc5c4F)cn3)nc2)CC(C)O1] |
| 491. | 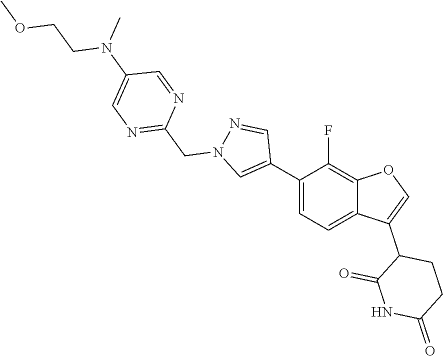 [SMILES: COCCN(C)c1cnc(Cn2cc(-c3ccc4c(C5CCC(=O)NC5=O)coc4c3F)cn2)nc1] |
| 492. | 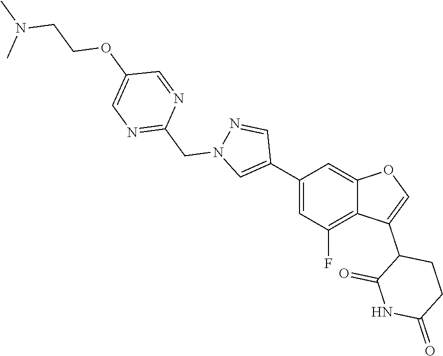 [SMILES: CN(C)CCOc1cnc(Cn2cc(-c3cc(F)c4c(C5CCC(=O)NC5=O)coc4c3)cn2)nc1] |
| 493. | 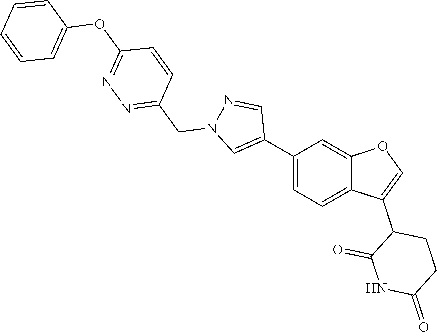 [SMILES: O=C1CCC(c2coc3cc(-c4cnn(Cc5ccc(Oc6ccccc6)nn5)c4)ccc23)C(=O)N1] |
| 494. | 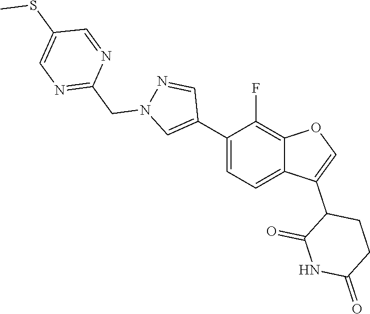 [SMILES: CSc1cnc(Cn2cc(-c3ccc4c(C5CCC(=O)NC5=O)coc4c3F)cn2)nc1] |
| 495. | 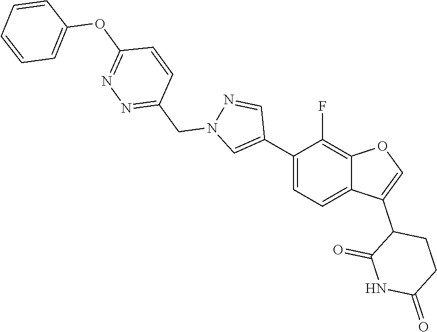 [SMILES: O=C1CCC(c2coc3c(F)c(-c4cnn(Cc5ccc(Oc6ccccc6)nn5)c4)ccc23)C(=O)N1] |
| 496. | 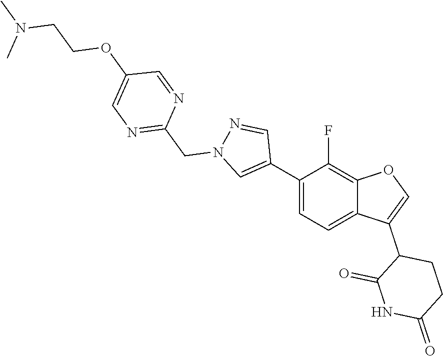 [SMILES: CN(C)CCOc1cnc(Cn2cc(-c3ccc4c(C5CCC(=O)NC5=O)coc4c3F)cn2)nc1] |
| 497. | 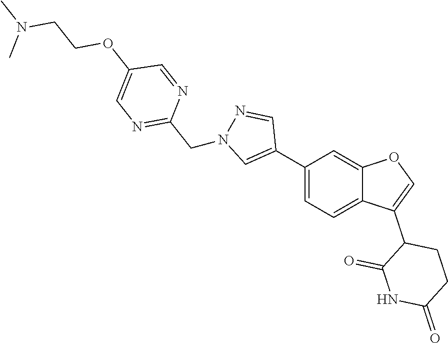 [SMILES: CN(C)CCOc1cnc(Cn2cc(-c3ccc4c(C5CCC(=O)NC5=O)coc4c3)cn2)nc1] |

TABLE B
Non-limiting selected compounds of the application

| Cmpd No. | Structure |
|---|---|
| 241. | 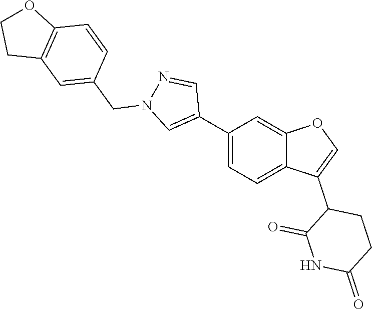 [SMILES: O=C1CCC(c2coc3cc(-c4cnn(Cc5ccc6c(c5)CCO6)c4)ccc23)C(=O)N1] |
| 239. | 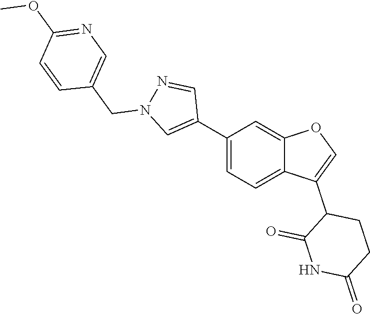 [SMILES: COc1ccc(Cn2cc(-c3ccc4c(C5CCC(=O)NC5=O)coc4c3)cn2)cn1] |
| 349. | 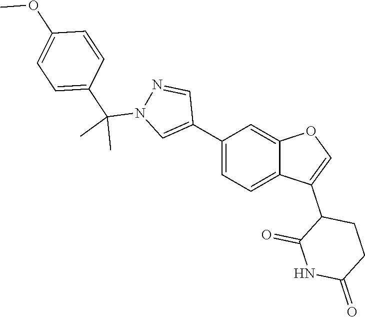 [SMILES: COc1ccc(C(C)(C)n2cc(-c3ccc4c(C5CCC(=O)NC5=O)coc4c3)cn2)cc1] |
| 348. | 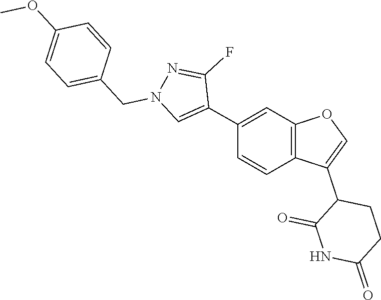 [SMILES: COc1ccc(Cn2cc(-c3ccc4c(C5CCC(=O)NC5=O)coc4c3)c(F)n2)cc1] |
| 42. | 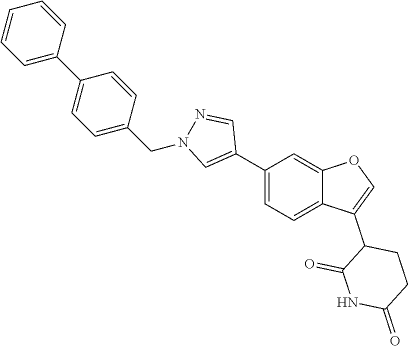 [SMILES: O=C1CCC(c2coc3cc(-c4cnn(Cc5ccc(-c6ccccc6)cc5)c4)ccc23)C(=O)N1] |
| 46. | 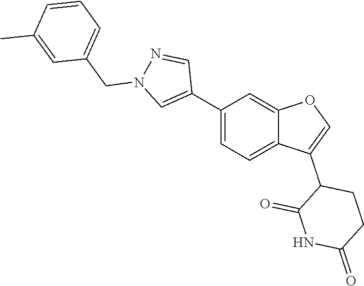 [SMILES: Cc1cccc(Cn2cc(-c3ccc4c(C5CCC(=O)NC5=O)coc4c3)cn2)c1] |
| 45. | 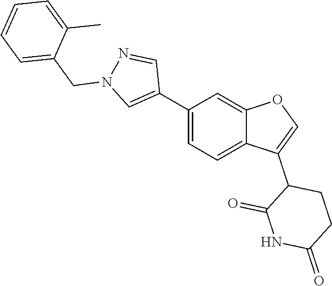 [SMILES: Cc1ccccc1Cn1cc(-c2ccc3c(C4CCC(=O)NC4=O)coc3c2)cn1] |
| 164. | 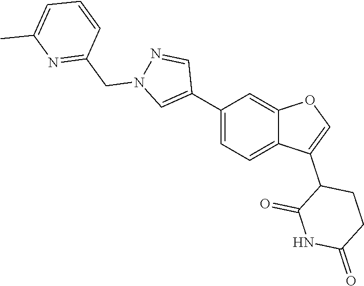 [SMILES: Cc1cccc(Cn2cc(-c3ccc4c(C5CCC(=O)NC5=O)coc4c3)cn2)n1] |
| 168. | 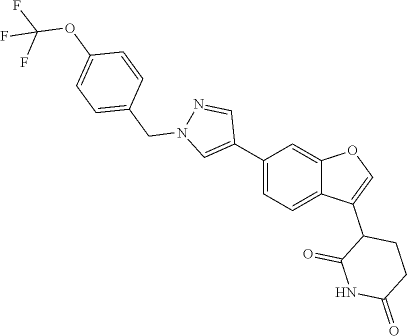 [SMILES: O=C1CCC(c2coc3cc(-c4cnn(Cc5ccc(OC(F)(F)F)cc5)c4)ccc23)C(=O)N1] |
| 108. | 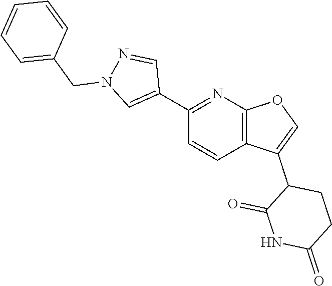 [SMILES: O=C1CCC(c2coc3nc(-c4cnn(Cc5ccccc5)c4)ccc23)C(=O)N1] |
| 172. | 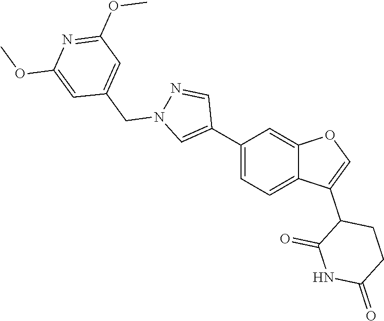 [SMILES: COc1cc(Cn2cc(-c3ccc4c(C5CCC(=O)NC5=O)coc4c3)cn2)cc(OC)n1] |
| 185. | 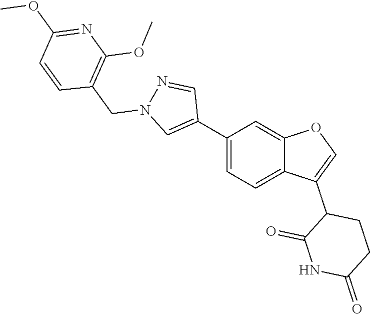 [SMILES: COc1ccc(Cn2cc(-c3ccc4c(C5CCC(=O)NC5=O)coc4c3)cn2)c(OC)n1] |
| 226. | 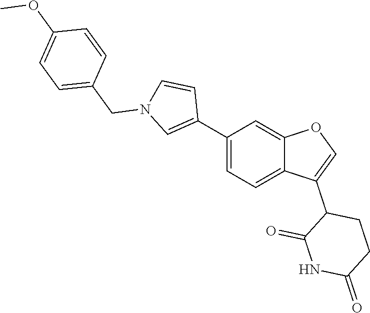 [SMILES: COc1ccc(Cn2ccc(-c3ccc4c(C5CCC(=O)NC5=O)coc4c3)c2)cc1] |
| 234. | 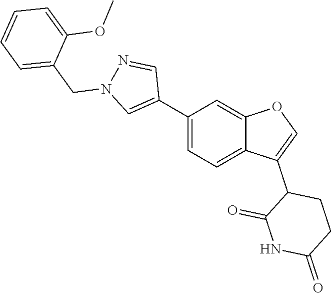 [SMILES: COc1ccccc1Cn1cc(-c2ccc3c(C4CCC(=O)NC4=O)coc3c2)cn1] |
| 236. | 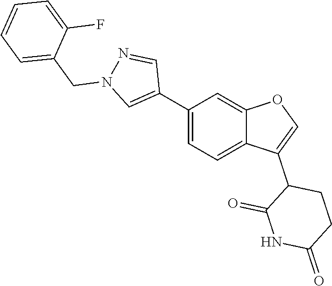 [SMILES: O=C1CCC(c2coc3cc(-c4cnn(Cc5ccccc5F)c4)ccc23)C(=O)N1] |
| 47. | 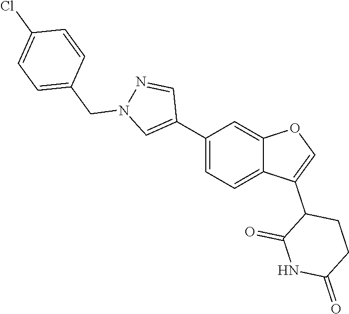 [SMILES: O=C1CCC(c2coc3cc(-c4cnn(Cc5ccc(Cl)cc5)c4)ccc23)C(=O)N1] |
| 258. | 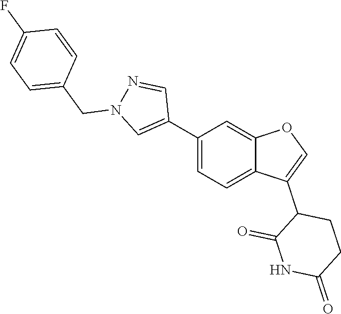 [SMILES: O=C1CCC(c2coc3cc(-c4cnn(Cc5ccc(F)cc5)c4)ccc23)C(=O)N1] |
| 341. | 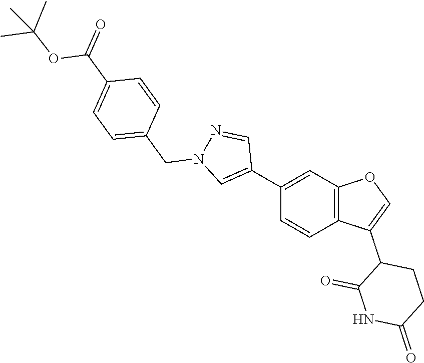 [SMILES: CC(C)(C)OC(=O)c1ccc(Cn2cc(-c3ccc4c(C5CCC(=O)NC5=O)coc4c3)cn2)cc1] |
| 323. | 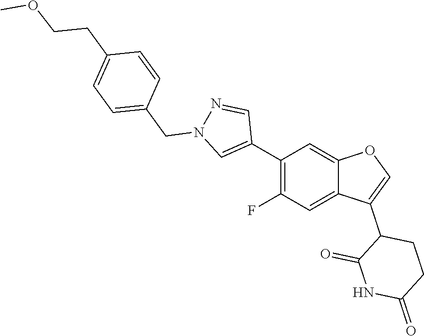 [SMILES: COCCc1ccc(Cn2cc(-c3cc4occ(C5CCC(=O)NC5=O)c4cc3F)cn2)cc1] |
| 197. | 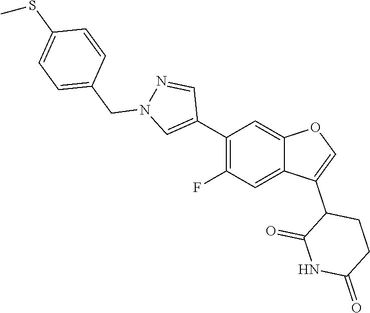 [SMILES: CSc1ccc(Cn2cc(-c3cc4occ(C5CCC(=O)NC5=O)c4cc3F)cn2)cc1] |
| 256. | 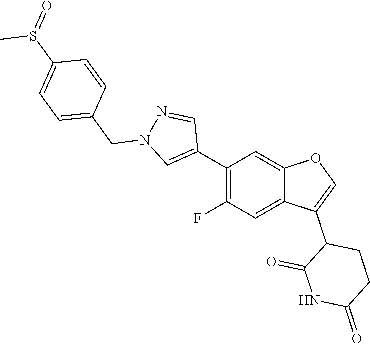 [SMILES: CS(=O)c1ccc(Cn2cc(-c3cc4occ(C5CCC(=O)NC5=O)c4cc3F)cn2)cc1] |
| 269. | 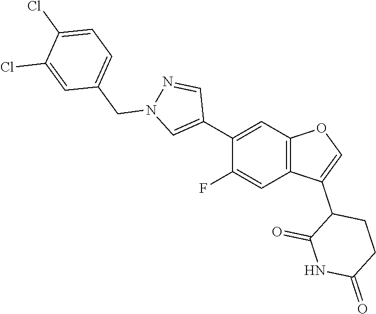 [SMILES: O=C1CCC(c2coc3cc(-c4cnn(Cc5ccc(Cl)c(Cl)c5)c4)c(F)cc23)C(=O)N1] |
| 284. | 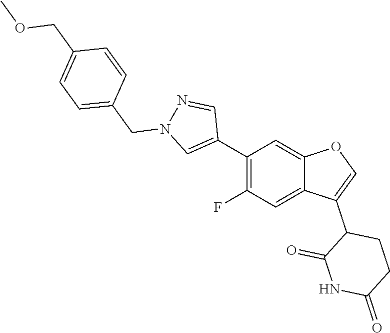 [SMILES: COCc1ccc(Cn2cc(-c3cc4occ(C5CCC(=O)NC5=O)c4cc3F)cn2)cc1] |
| 289. | 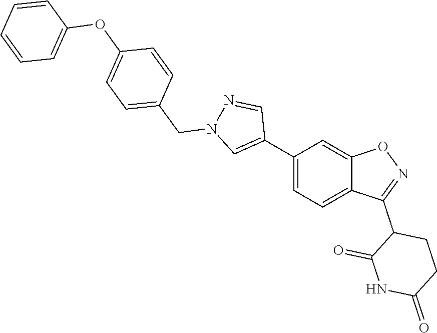 [SMILES: O=C1CCC(c2noc3cc(-c4cnn(Cc5ccc(Oc6ccccc6)cc5)c4)ccc23)C(=O)N1] |
| 122. | 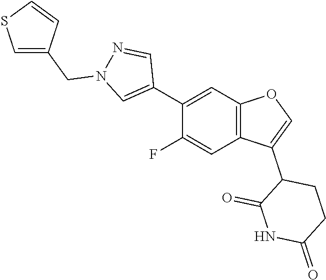 [SMILES: O=C1CCC(c2coc3cc(-c4cnn(Cc5ccsc5)c4)c(F)cc23)C(=O)N1] |
| 10. | 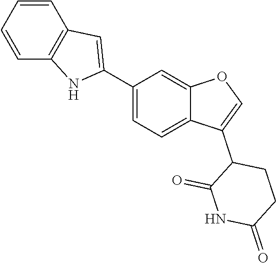 [SMILES: O=C1CCC(c2coc3cc(-c4cc5ccccc5[nH]4)ccc23)C(=O)N1] |
| 44. | 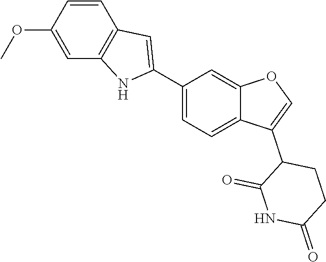 [SMILES: COc1ccc2cc(-c3ccc4c(C5CCC(=O)NC5=O)coc4c3)[nH]c2c1] |
| 49. | 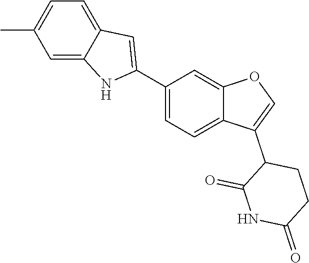 [SMILES: Cc1ccc2cc(-c3ccc4c(C5CCC(=O)NC5=O)coc4c3)[nH]c2c1] |
| 60. | 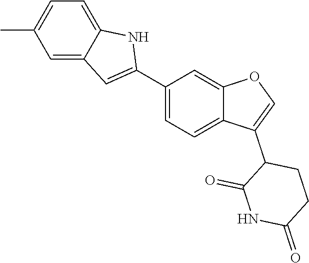 [SMILES: Cc1ccc2[nH]c(-c3ccc4c(C5CCC(=O)NC5=O)coc4c3)cc2c1] |
| 85. | 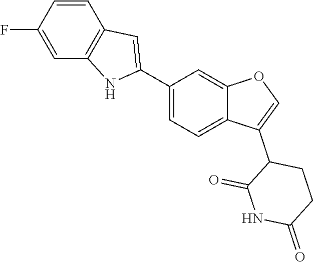 [SMILES: O=C1CCC(c2coc3cc(-c4cc5ccc(F)cc5[nH]4)ccc23)C(=O)N1] |
| 86. | 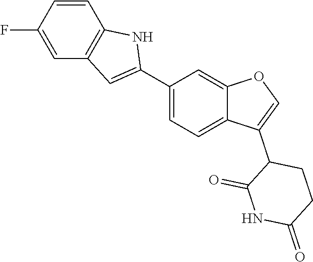 [SMILES: O=C1CCC(c2coc3cc(-c4cc5cc(F)ccc5[nH]4)ccc23)C(=O)N1] |
| 89. | 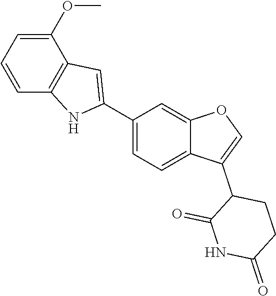 [SMILES: COc1cccc2[nH]c(-c3ccc4c(C5CCC(=O)NC5=O)coc4c3)cc12] |
| 61. | 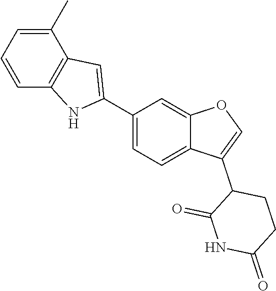 [SMILES: Cc1cccc2[nH]c(-c3ccc4c(C5CCC(=O)NC5=O)coc4c3)cc12] |
| 311. | 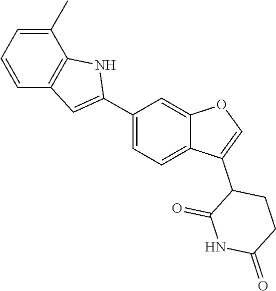 [SMILES: Cc1cccc2cc(-c3ccc4c(C5CCC(=O)NC5=O)coc4c3)[nH]c12] |
| 50. | 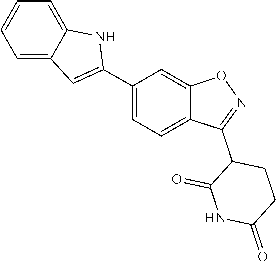 [SMILES: O=C1CCC(c2noc3cc(-c4cc5ccccc5[nH]4)ccc23)C(=O)N1] |
| 58. | 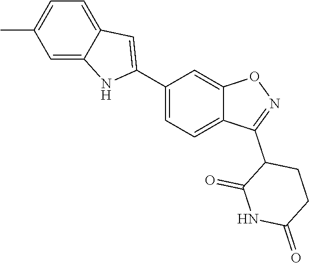 [SMILES: Cc1ccc2cc(-c3ccc4c(C5CCC(=O)NC5=O)noc4c3)[nH]c2c1] |
| 167. | 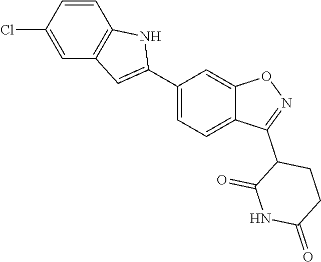 [SMILES: O=C1CCC(c2noc3cc(-c4cc5cc(Cl)ccc5[nH]4)ccc23)C(=O)N1] |
| 355. | 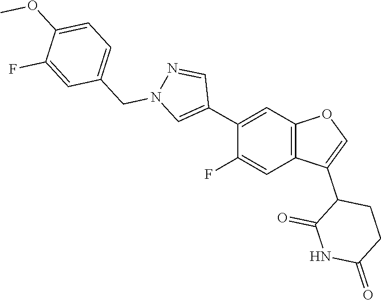 [SMILES: COc1ccc(Cn2cc(-c3cc4occ(C5CCC(=O)NC5=O)c4cc3F)cn2)cc1F] |
| 360. | 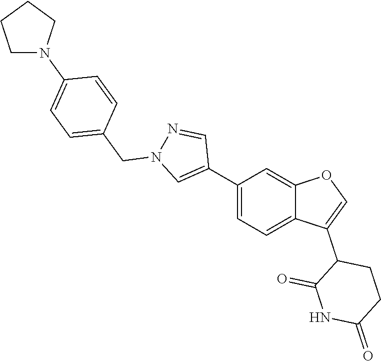 [SMILES: O=C1CCC(c2coc3cc(-c4cnn(Cc5ccc(N6CCCC6)cc5)c4)ccc23)C(=O)N1] |
| 364. | 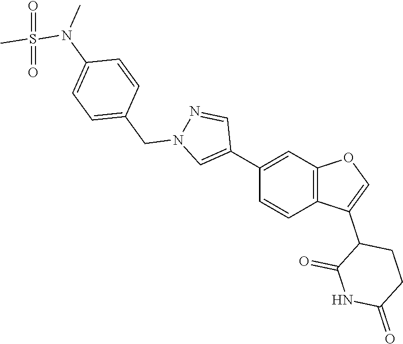 [SMILES: CN(c1ccc(Cn2cc(-c3ccc4c(C5CCC(=O)NC5=O)coc4c3)cn2)cc1)S(C)(=O)=O] |
| 365. | 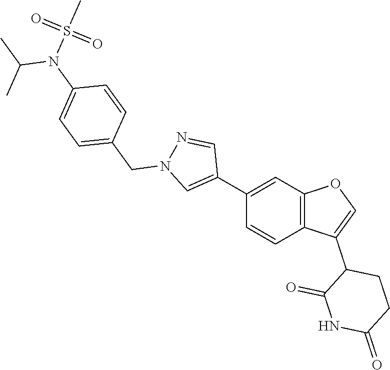 [SMILES: CC(C)N(c1ccc(Cn2cc(-c3ccc4c(C5CCC(=O)NC5=O)coc4c3)cn2)cc1)S(C)(=O)=O] |
| 371. | 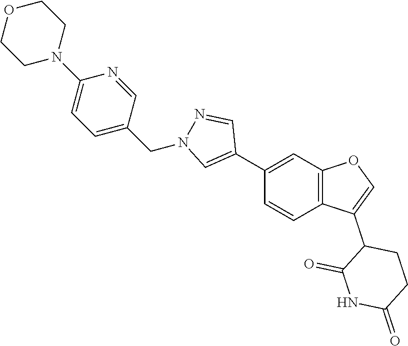 [SMILES: O=C1CCC(c2coc3cc(-c4cnn(Cc5ccc(N6CCOCC6)nc5)c4)ccc23)C(=O)N1] |
| 372. | 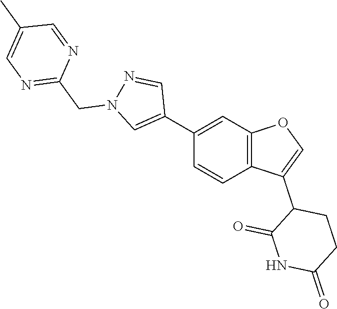 [SMILES: Cc1cnc(Cn2cc(-c3ccc4c(C5CCC(=O)NC5=O)coc4c3)cn2)nc1] |
| 373. | 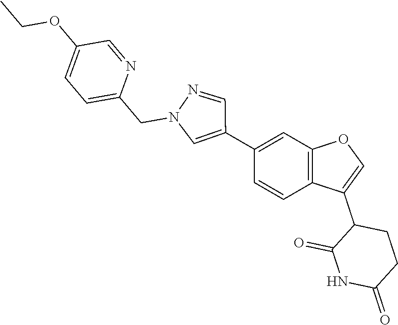 [SMILES: CCOc1ccc(Cn2cc(-c3ccc4c(C5CCC(=O)NC5=O)coc4c3)cn2)nc1] |
| 376. | 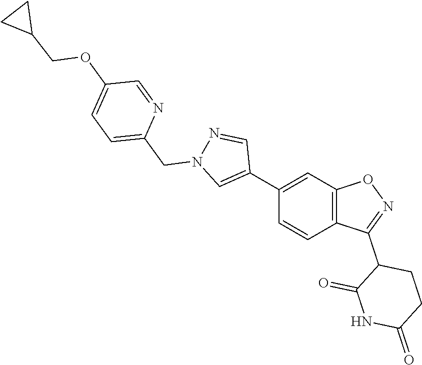 [SMILES: O=C1CCC(c2noc3cc(-c4cnn(Cc5ccc(OCC6CC6)cn5)c4)ccc23)C(=O)N1] |
| 377. | 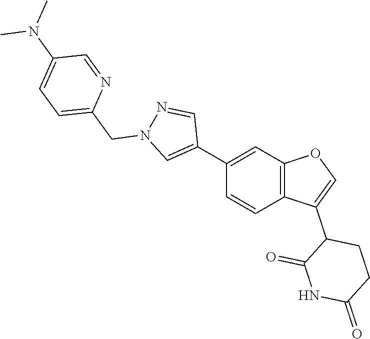 [SMILES: CN(C)c1ccc(Cn2cc(-c3ccc4c(C5CCC(=O)NC5=O)coc4c3)cn2)nc1] |
| 379. | 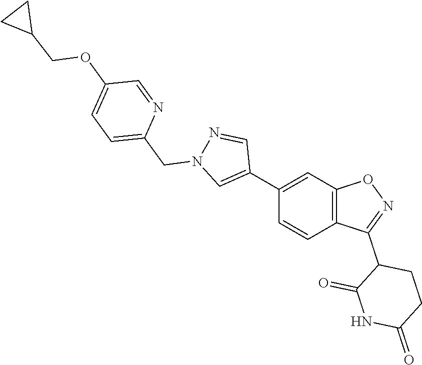 [SMILES: O=C1CCC(c2noc3cc(-c4cnn(Cc5ccc(OCC6CC6)cn5)c4)ccc23)C(=O)N1] |
| 384. | 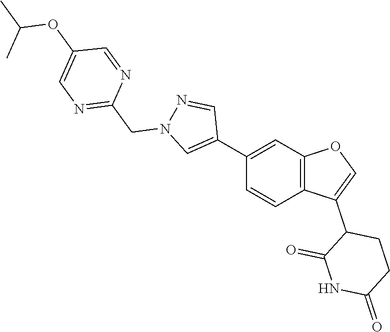 [SMILES: CC(C)Oc1cnc(Cn2cc(-c3ccc4c(C5CCC(=O)NC5=O)coc4c3)cn2)nc1] |
| 386. | 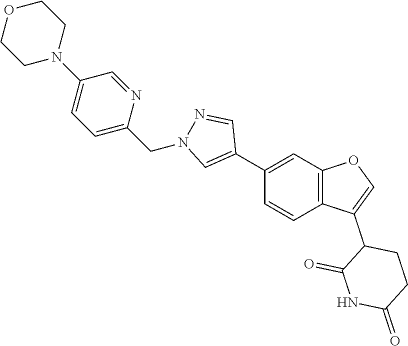 [SMILES: O=C1CCC(c2coc3cc(-c4cnn(Cc5ccc(N6CCOCC6)cn5)c4)ccc23)C(=O)N1] |
| 387. | 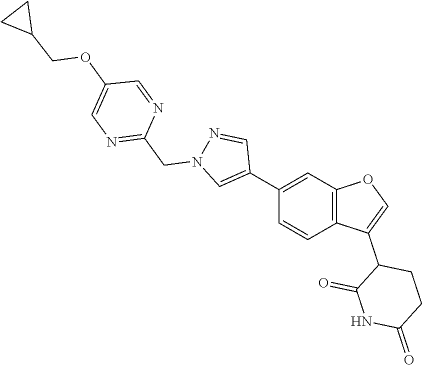 [SMILES: O=C1CCC(c2coc3cc(-c4cnn(Cc5ncc(OCC6CC6)cn5)c4)ccc23)C(=O)N1] |
| 388. | 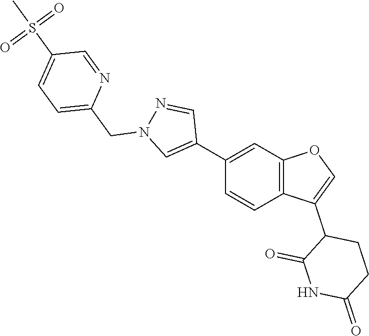 [SMILES: CS(=O)(=O)c1ccc(Cn2cc(-c3ccc4c(C5CCC(=O)NC5=O)coc4c3)cn2)nc1] |
| 392. | 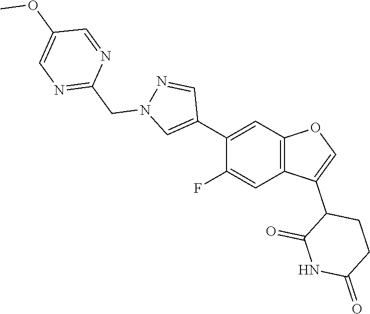 [SMILES: COc1cnc(Cn2cc(-c3cc4occ(C5CCC(=O)NC5=O)c4cc3F)cn2)nc1] |
| 393. | 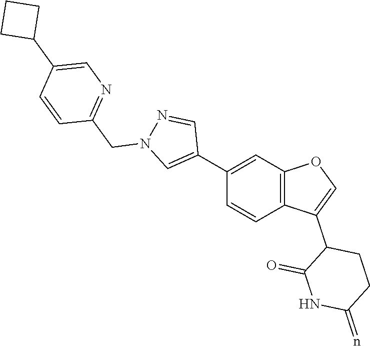 |
| 395. | 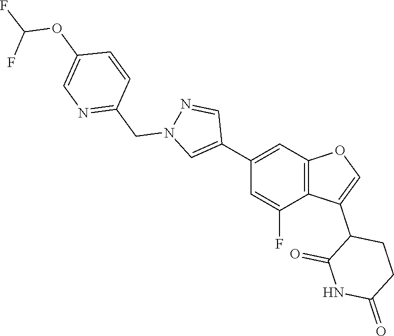 [SMILES: O=C1CCC(c2coc3cc(-c4cnn(Cc5ccc(OC(F)F)cn5)c4)cc(F)c23)C(=O)N1] |
| 396. | 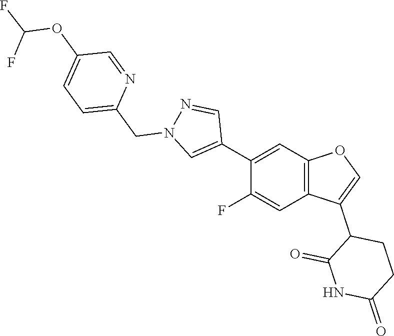 [SMILES: O=C1CCC(c2coc3cc(-c4cnn(Cc5ccc(OC(F)F)cn5)c4)c(F)cc23)C(=O)N1] |
| 397. | 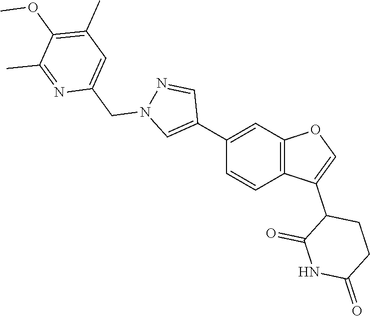 [SMILES: COc1c(C)cc(Cn2cc(-c3ccc4c(C5CCC(=O)NC5=O)coc4c3)cn2)nc1C] |
| 398. | 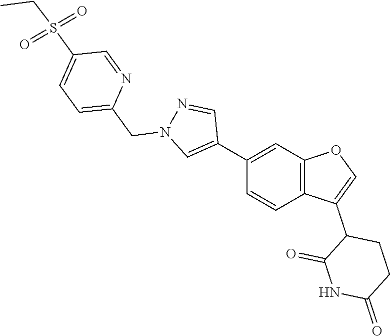 [SMILES: CCS(=O)(=O)c1ccc(Cn2cc(-c3ccc4c(C5CCC(=O)NC5=O)coc4c3)cn2)nc1] |
| 401. | 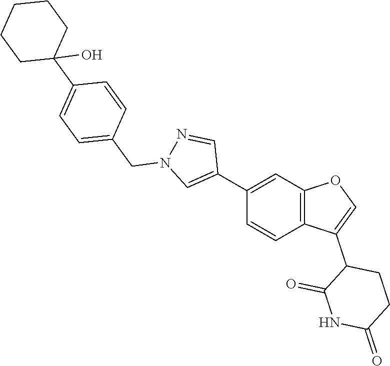 [SMILES: O=C1CCC(c2coc3cc(-c4cnn(Cc5ccc(C6(O)CCCCC6)cc5)c4)ccc23)C(=O)N1] |
| 406. | 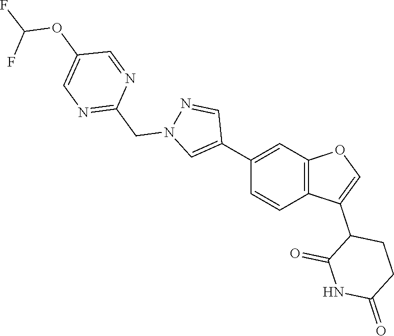 [SMILES: O=C1CCC(c2coc3cc(-c4cnn(Cc5ncc(OC(F)F)cn5)c4)ccc23)C(=O)N1] |
| 407. | 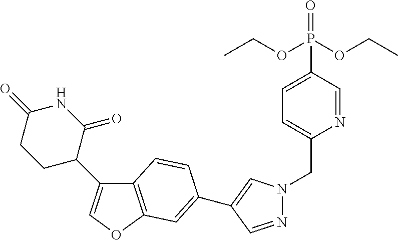 [SMILES: CCOP(=O)(OCC)c1ccc(Cn2cc(-c3ccc4c(C5CCC(=O)NC5=O)coc4c3)cn2)nc1] |
| 412. | 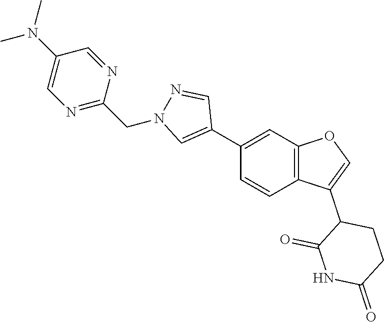 [SMILES: CN(C)c1cnc(Cn2cc(-c3ccc4c(C5CCC(=O)NC5=O)coc4c3)cn2)nc1] |
| 413. | 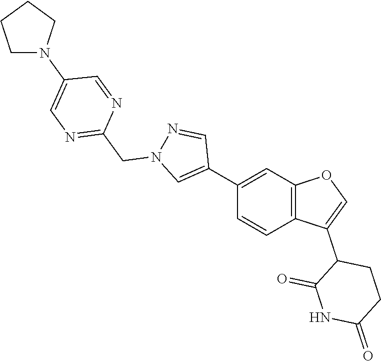 [SMILES: O=C1CCC(c2coc3cc(-c4cnn(Cc5ncc(N6CCCC6)cn5)c4)ccc23)C(=O)N1] |
| 414. | 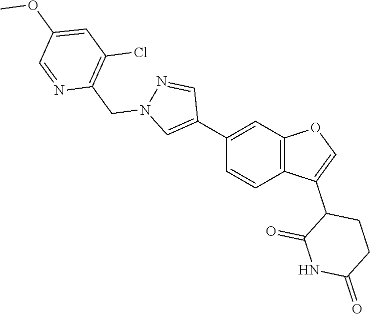 [SMILES: COc1cnc(Cn2cc(-c3ccc4c(C5CCC(=O)NC5=O)coc4c3)cn2)c(Cl)c1] |
| 416. | 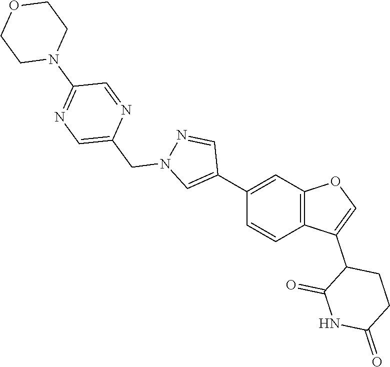 [SMILES: O=C1CCC(c2coc3cc(-c4cnn(Cc5cnc(N6CCOCC6)cn5)c4)ccc23)C(=O)N1] |
| 417. | 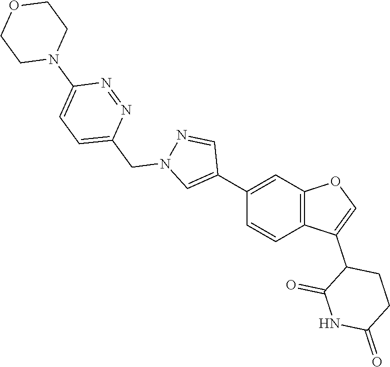 [SMILES: O=C1CCC(c2coc3cc(-c4cnn(Cc5ccc(N6CCOCC6)nn5)c4)ccc23)C(=O)N1] |
| 418. | 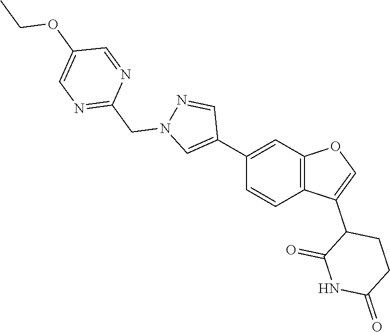 [SMILES: CCOc1cnc(Cn2cc(-c3ccc4c(C5CCC(=O)NC5=O)coc4c3)cn2)nc1] |
| 419. | 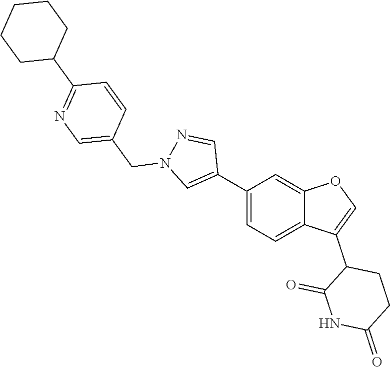 [SMILES: O=C1CCC(c2coc3cc(-c4cnn(Cc5ccc(C6CCCCC6)nc5)c4)ccc23)C(=O)N1] |
| 421. | 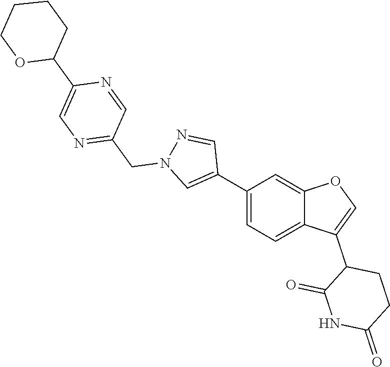 [SMILES: O=C1CCC(c2coc3cc(-c4cnn(Cc5cnc(C6CCCCO6)cn5)c4)ccc23)C(=O)N1] |
| 422. | 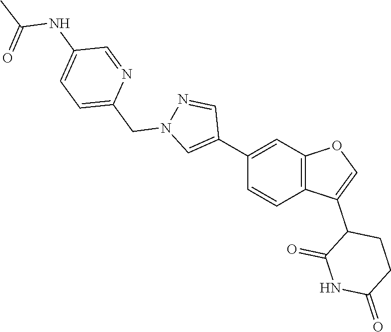 [SMILES: CC(=O)Nc1ccc(Cn2cc(-c3ccc4c(C5CCC(=O)NC5=O)coc4c3)cn2)nc1] |
| 423. | 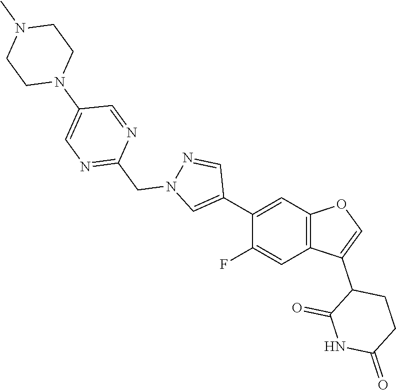 [SMILES: CN1CCN(c2cnc(Cn3cc(-c4cc5occ(C6CCC(=O)NC6=O)c5cc4F)cn3)nc2)CC1] |
| 425. | 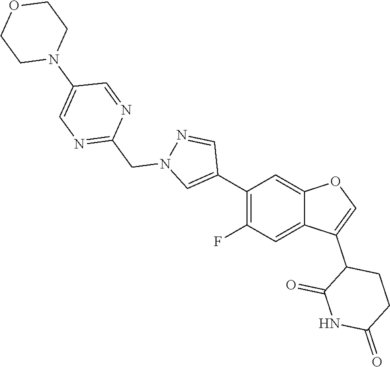 [SMILES: O=C1CCC(c2coc3cc(-c4cnn(Cc5ncc(N6CCOCC6)cn5)c4)c(F)cc23)C(=O)N1] |
| 427. | 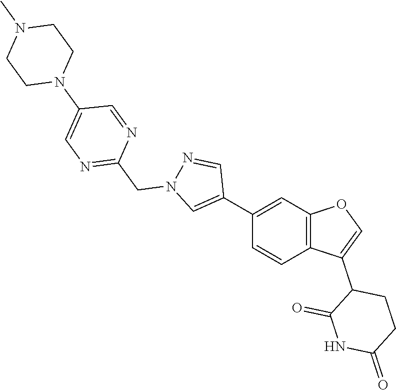 [SMILES: CN1CCN(c2cnc(Cn3cc(-c4ccc5c(C6CCC(=O)NC6=O)coc5c4)cn3)nc2)CC1] |
| 430. | 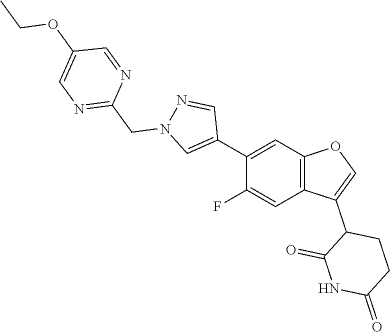 [SMILES: CCOc1cnc(Cn2cc(-c3cc4occ(C5CCC(=O)NC5=O)c4cc3F)cn2)nc1] |
| 432. | 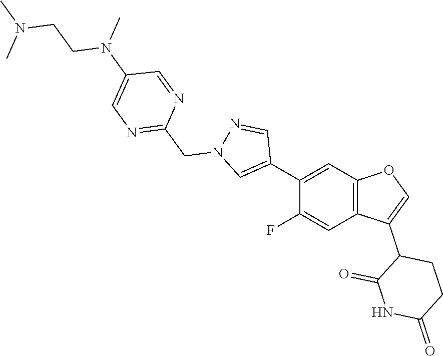 [SMILES: CN(C)CCN(C)c1cnc(Cn2cc(-c3cc4occ(C5CCC(=O)NC5=O)c4cc3F)cn2)nc1] |
| 433. | 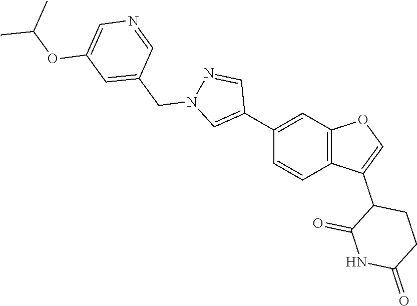 [SMILES: CC(C)Oc1cncc(Cn2cc(-c3ccc4c(C5CCC(=O)NC5=O)coc4c3)cn2)c1] |
| 434. | 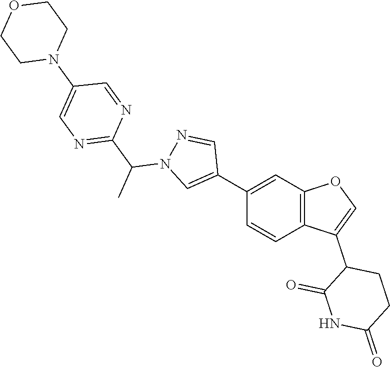 [SMILES: CC(c1ncc(N2CCOCC2)cn1)n1cc(-c2ccc3c(C4CCC(=O)NC4=O)coc3c2)cn1] |
| 435. | 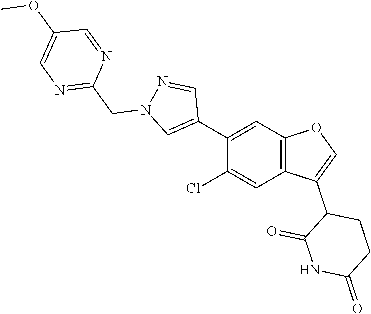 [SMILES: COc1cnc(Cn2cc(-c3cc4occ(C5CCC(=O)NC5=O)c4cc3Cl)cn2)nc1] |
| 436. | 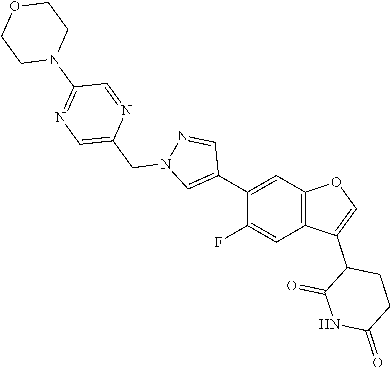 [SMILES: O=C1CCC(c2coc3cc(-c4cnn(Cc5cnc(N6CCOCC6)cn5)c4)c(F)cc23)C(=O)N1] |
| 437. | 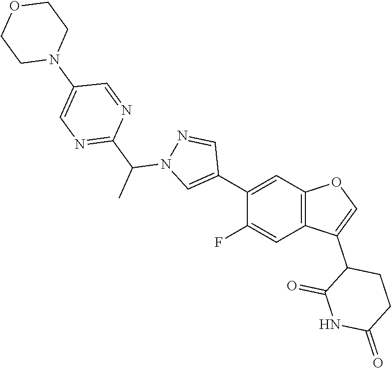 [SMILES: CC(c1ncc(N2CCOCC2)cn1)n1cc(-c2cc3occ(C4CCC(=O)NC4=O)c3cc2F)cn1] |
| 438. | 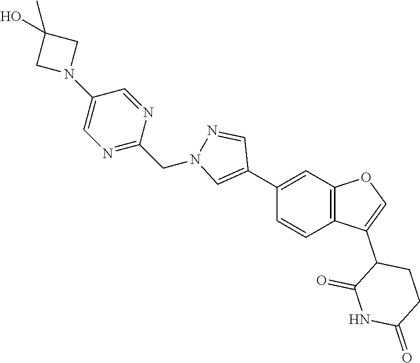 [SMILES: CC1(O)CN(c2cnc(Cn3cc(-c4ccc5c(C6CCC(=O)NC6=O)coc5c4)cn3)nc2)C1] |
| 439. | 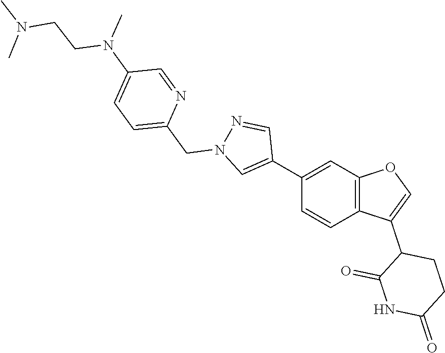 [SMILES: CN(C)CCN(C)c1ccc(Cn2cc(-c3ccc4c(C5CCC(=O)NC5=O)coc4c3)cn2)nc1] |
| 440. | 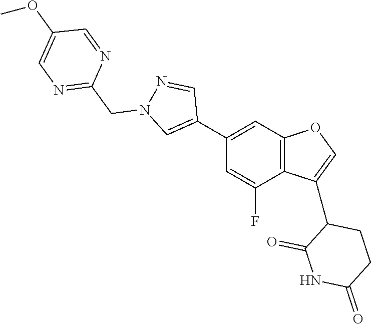 [SMILES: COc1cnc(Cn2cc(-c3cc(F)c4c(C5CCC(=O)NC5=O)coc4c3)cn2)nc1] |
| 442. | 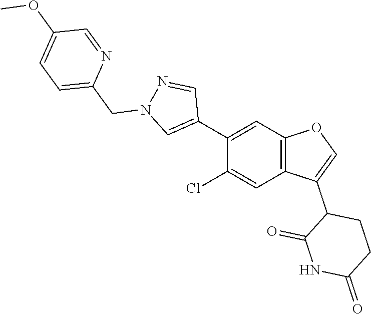 [SMILES: COc1ccc(Cn2cc(-c3cc4occ(C5CCC(=O)NC5=O)c4cc3Cl)cn2)nc1] |
| 443. | 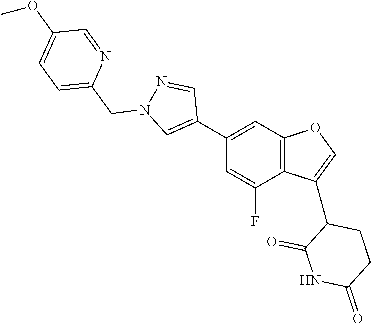 [SMILES: COc1ccc(Cn2cc(-c3cc(F)c4c(C5CCC(=O)NC5=O)coc4c3)cn2)nc1] |
| 444. | 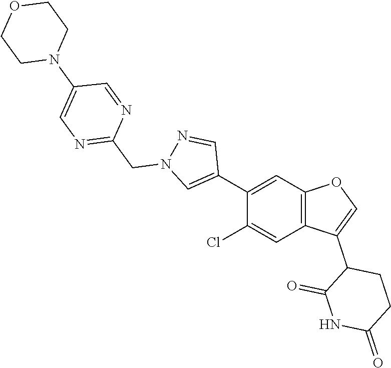 [SMILES: O=C1CCC(c2coc3cc(-c4cnn(Cc5ncc(N6CCOCC6)cn5)c4)c(Cl)cc23)C(=O)N1] |
| 446. | 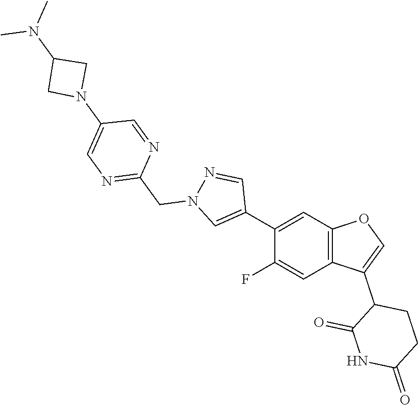 [SMILES: CN(C)C1CN(c2cnc(Cn3cc(-c4cc5occ(C6CCC(=O)NC6=O)c5cc4F)cn3)nc2)C1] |
| 447. | 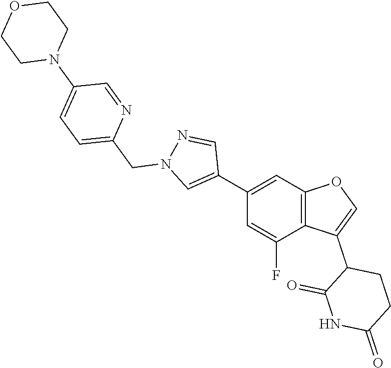 [SMILES: O=C1CCC(c2coc3cc(-c4cnn(Cc5ccc(N6CCOCC6)cn5)c4)cc(F)c23)C(=O)N1] |
| 448. | 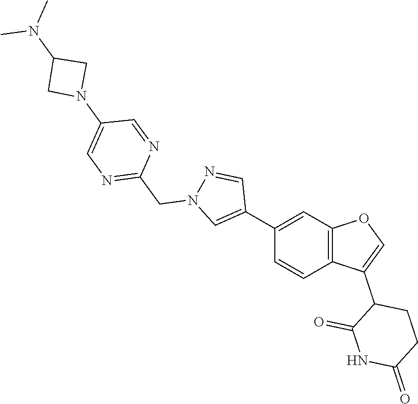 [SMILES: CN(C)C1CN(c2cnc(Cn3cc(-c4ccc5c(C6CCC(=O)NC6=O)coc5c4)cn3)nc2)C1] |
| 449. | 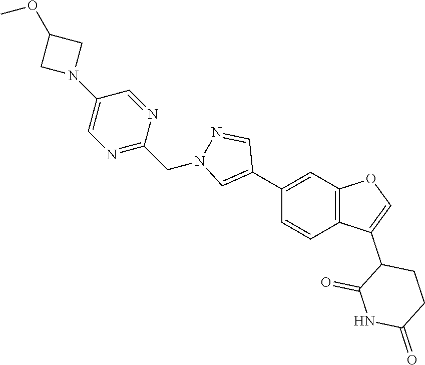 [SMILES: COC1CN(c2cnc(Cn3cc(-c4ccc5c(C6CCC(=O)NC6=O)coc5c4)cn3)nc2)C1] |
| 450. | 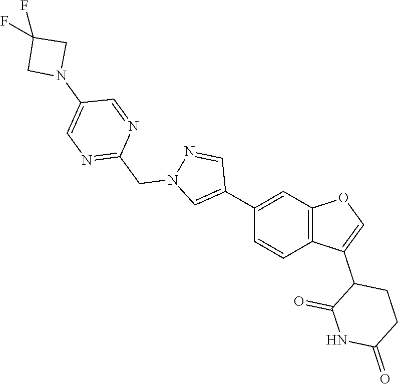 [SMILES: O=C1CCC(c2coc3cc(-c4cnn(Cc5ncc(N6CC(F)(F)C6)cn5)c4)ccc23)C(=O)N1] |
| 451. | 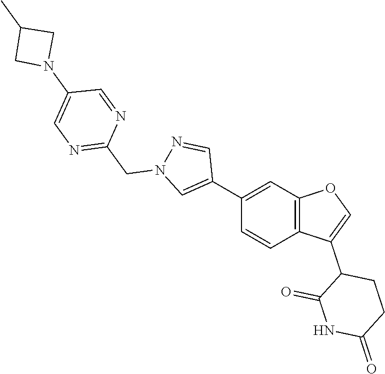 [SMILES: CC1CN(c2cnc(Cn3cc(-c4ccc5c(C6CCC(=O)NC6=O)coc5c4)cn3)nc2)C1] |
| 452. | 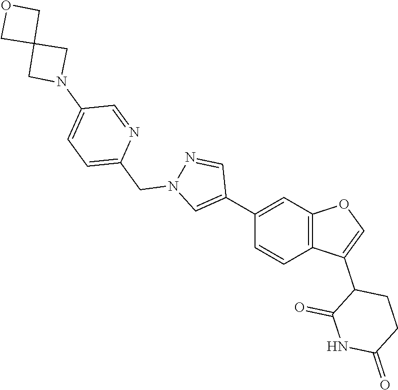 [SMILES: O=C1CCC(c2coc3cc(-c4cnn(Cc5ccc(N6CC7(COC7)C6)cn5)c4)ccc23)C(=O)N1] |
| 453. | 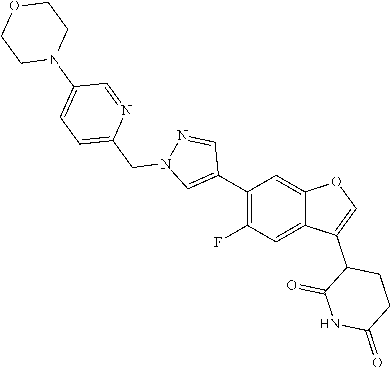 [SMILES: O=C1CCC(c2coc3cc(-c4cnn(Cc5ccc(N6CCOCC6)cn5)c4)c(F)cc23)C(=O)N1] |
| 454. | 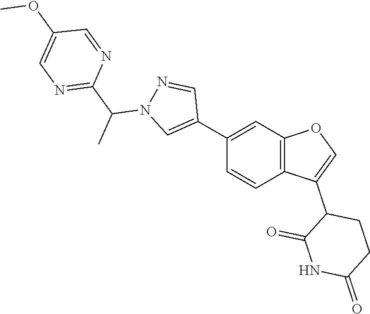 [SMILES: COc1cnc(C(C)n2cc(-c3ccc4c(C5CCC(=O)NC5=O)coc4c3)cn2)nc1] |
| 455. | 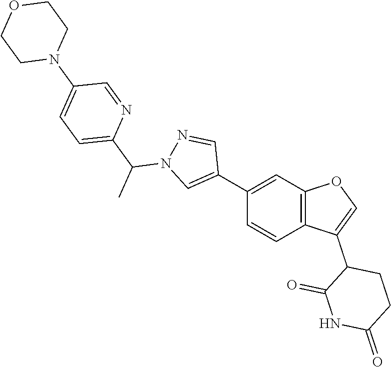 [SMILES: CC(c1ccc(N2CCOCC2)cn1)n1cc(-c2ccc3c(C4CCC(=O)NC4=O)coc3c2)cn1] |
| 457. | 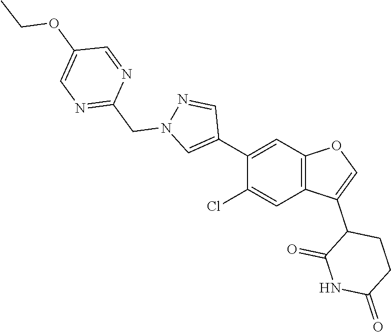 [SMILES: CCOc1cnc(Cn2cc(-c3cc4occ(C5CCC(=O)NC5=O)c4cc3Cl)cn2)nc1] |
| 458. | 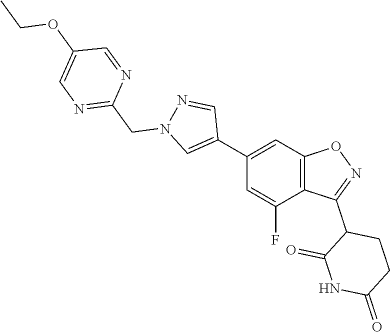 [SMILES: CCOc1cnc(Cn2cc(-c3cc(F)c4c(C5CCC(=O)NC5=O)noc4c3)cn2)nc1] |
| 459. | 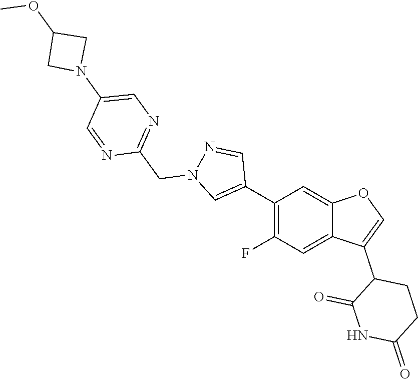 [SMILES: COC1CN(c2cnc(Cn3cc(-c4cc5occ(C6CCC(=O)NC6=O)c5cc4F)cn3)nc2)C1] |
| 461. | 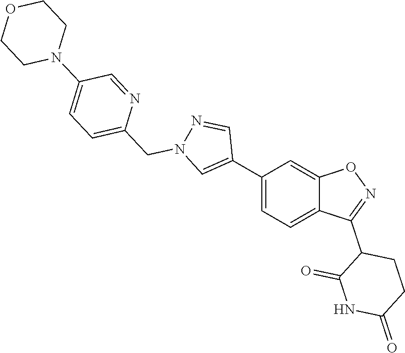 [SMILES: O=C1CCC(c2noc3cc(-c4cnn(Cc5ccc(N6CCOCC6)cn5)c4)ccc23)C(=O)N1] |
| 462. | 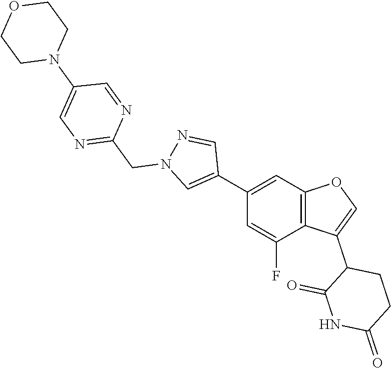 [SMILES: O=C1CCC(c2coc3cc(-c4cnn(Cc5ncc(N6CCOCC6)cn5)c4)cc(F)c23)C(=O)N1] |
| 463. | 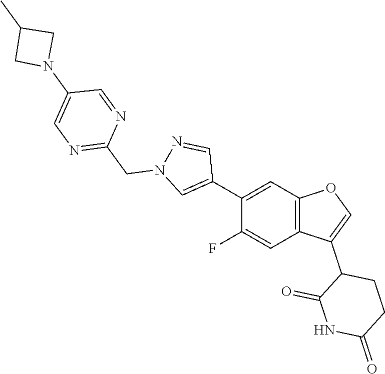 [SMILES: CC1CN(c2cnc(Cn3cc(-c4cc5occ(C6CCC(=O)NC6=O)c5cc4F)cn3)nc2)C1] |
| 465. | 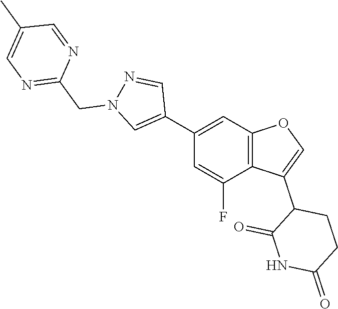 [SMILES: Cc1cnc(Cn2cc(-c3cc(F)c4c(C5CCC(=O)NC5=O)coc4c3)cn2)nc1] |
| 466. | 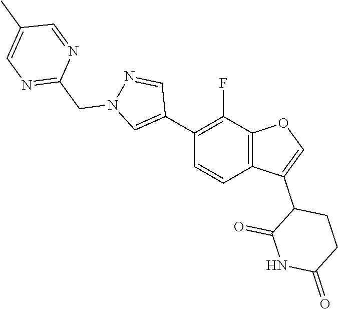 [SMILES: Cc1cnc(Cn2cc(-c3ccc4c(C5CCC(=O)NC5=O)coc4c3F)cn2)nc1] |
| 470. | 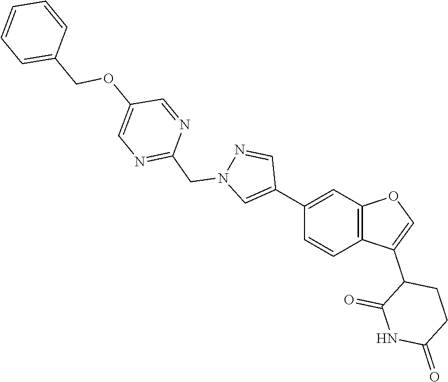 [SMILES: O=C1CCC(c2coc3cc(-c4cnn(Cc5ncc(OCc6ccccc6)cn5)c4)ccc23)C(=O)N1] |
| 471. | 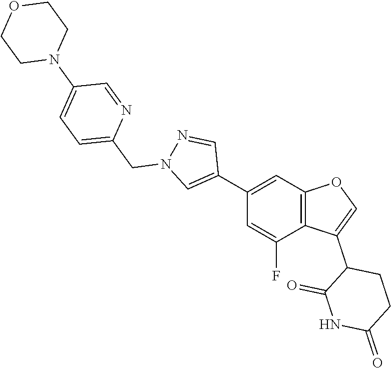 [SMILES: O=C1CCC(c2coc3cc(-c4cnn(Cc5ccc(N6CCOCC6)cn5)c4)cc(F)c23)C(=O)N1] |
| 472. | 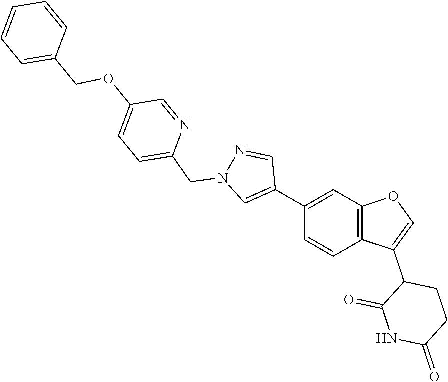 [SMILES: O=C1CCC(c2coc3cc(-c4cnn(Cc5ccc(OCc6ccccc6)cn5)c4)ccc23)C(=O)N1] |
| 473. | 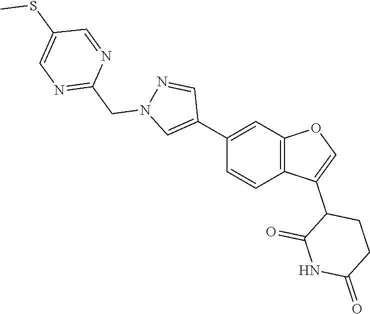 [SMILES: CSc1cnc(Cn2cc(-c3ccc4c(C5CCC(=O)NC5=O)coc4c3)cn2)nc1] |
| 474. | 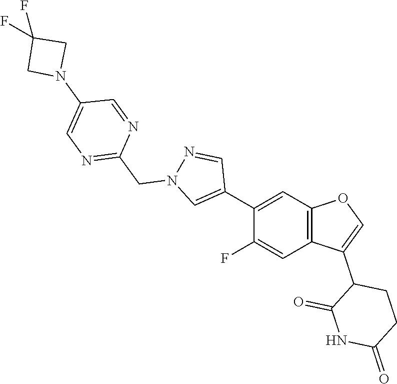 [SMILES: O=C1CCC(c2coc3cc(-c4cnn(Cc5ncc(N6CC(F)(F)C6)cn5)c4)c(F)cc23)C(=O)N1] |
| 477. | 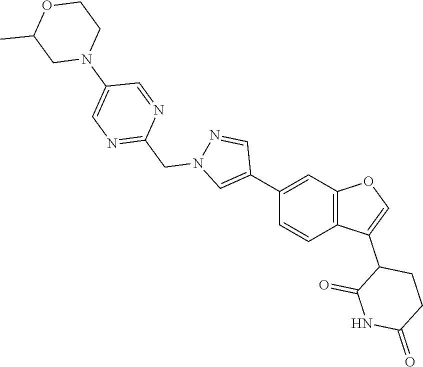 [SMILES: CC1CN(c2cnc(Cn3cc(-c4ccc5c(C6CCC(=O)NC6=O)coc5c4)cn3)nc2)CCO1] |
| 478. | 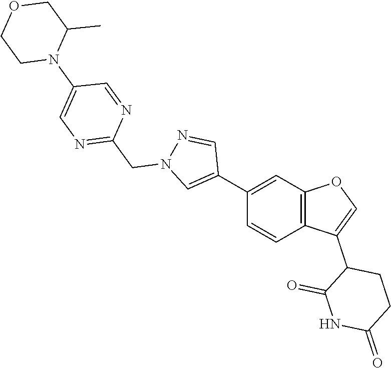 [SMILES: CC1COCCN1c1cnc(Cn2cc(-c3ccc4c(C5CCC(=O)NC5=O)coc4c3)cn2)nc1] |
| 482. | 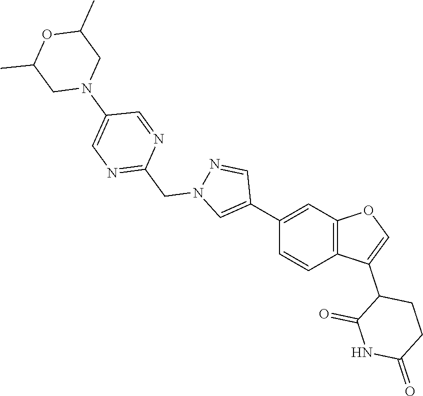 [SMILES: CC1CN(c2cnc(Cn3cc(-c4ccc5c(C6CCC(=O)NC6=O)coc5c4)cn3)nc2)CC(C)O1] |
| 488. | 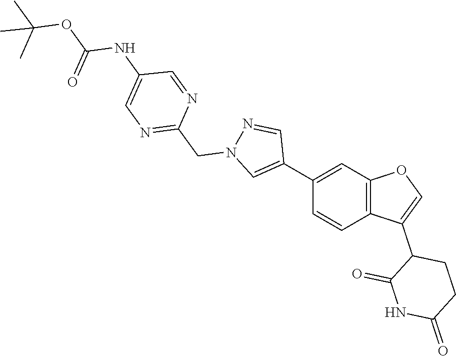 [SMILES: CC(C)(C)OC(=O)Nc1cnc(Cn2cc(-c3ccc4c(C5CCC(=O)NC5=O)coc4c3)cn2)nc1] |
| 490. | 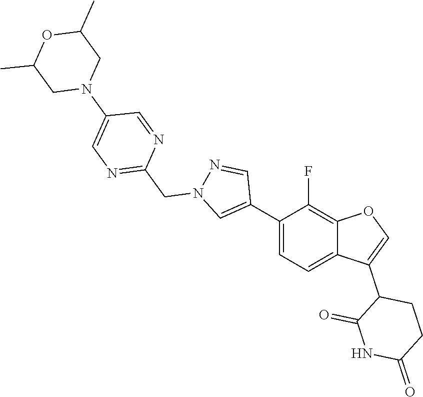 [SMILES: CC1CN(c2cnc(Cn3cc(-c4ccc5c(C6CCC(=O)NC6=O)coc5c4F)cn3)nc2)CC(C)O1] |
| 492. | 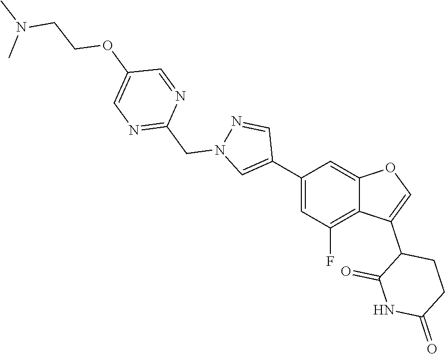 [SMILES: CN(C)CCOc1cnc(Cn2cc(-c3cc(F)c4c(C5CCC(=O)NC5=O)coc4c3)cn2)nc1] |
| 493. | 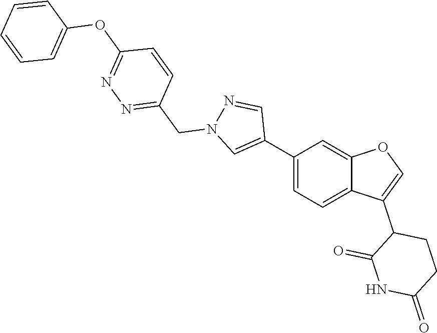 [SMILES: O=C1CCC(c2coc3cc(-c4cnn(Cc5ccc(Oc6ccccc6)nn5)c4)ccc23)C(=O)N1] |
| 494. | 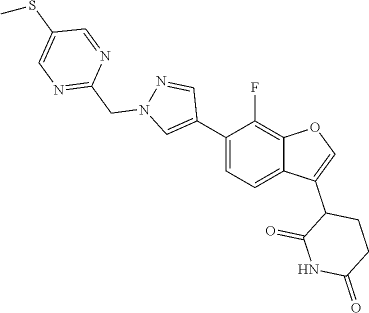 [SMILES: CSc1cnc(Cn2cc(-c3ccc4c(C5CCC(=O)NC5=O)coc4c3F)cn2)nc1] |
| 495. | 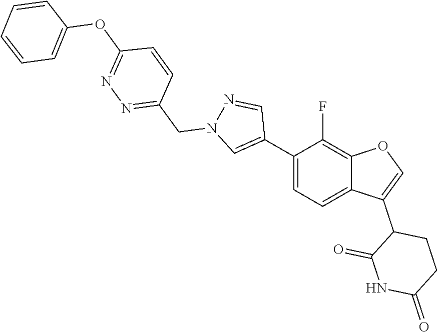 [SMILES: O=C1CCC(c2coc3c(F)c(-c4cnn(Cc5ccc(Oc6ccccc6)nn5)c4)ccc23)C(=O)N1] |
| 497. | 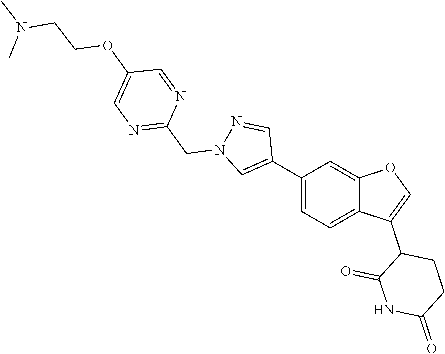 [SMILES: CN(C)CCOc1cnc(Cn2cc(-c3ccc4c(C5CCC(=O)NC5=O)coc4c3)cn2)nc1] |

TABLE C
Non-limiting selected compounds of the application

| Cmpd No. | Structure |
|---|---|
| 32. | 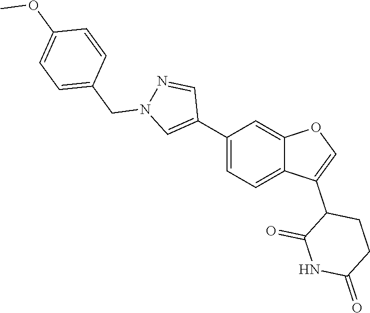 [SMILES: COc1ccc(Cn2cc(-c3ccc4c(C5CCC(=O)NC5=O)coc4c3)cn2)cc1] |
| 184. | 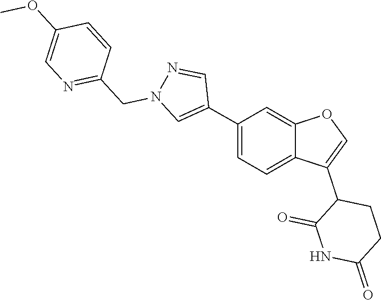 [SMILES: COc1ccc(Cn2cc(-c3ccc4c(C5CCC(=O)NC5=O)coc4c3)cn2)nc1] |
| 347. | 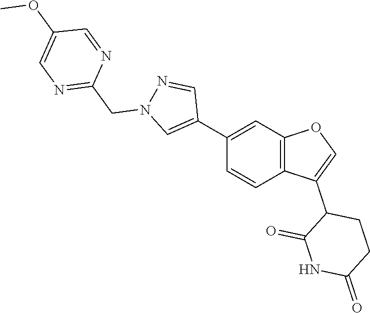 [SMILES: COc1cnc(Cn2cc(-c3ccc4c(C5CCC(=O)NC5=O)coc4c3)cn2)nc1] |
| 333. | 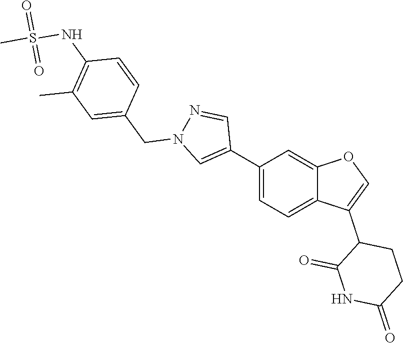 [SMILES: Cc1cc(Cn2cc(-c3ccc4c(C5CCC(=O)NC5=O)coc4c3)cn2)ccc1NS(C)(=O)=O] |
| 334. | 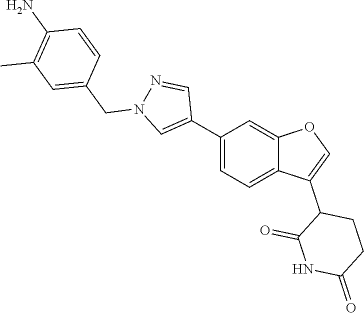 [SMILES: Cc1cc(Cn2cc(-c3ccc4c(C5CCC(=O)NC5=O)coc4c3)cn2)ccc1N] |
| 346. | 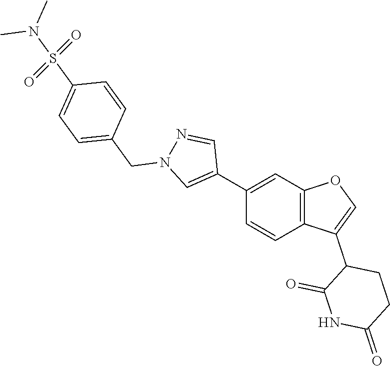 [SMILES: CN(C)S(=O)(=O)c1ccc(Cn2cc(-c3ccc4c(C5CCC(=O)NC5=O)coc4c3)cn2)cc1] |
| 270. | 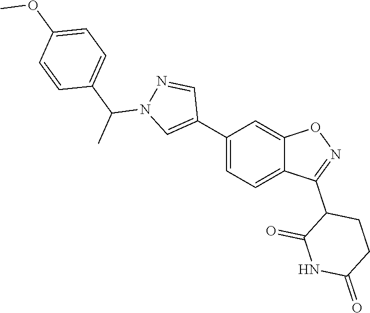 [SMILES: COc1ccc(C(C)n2cc(-c3ccc4c(C5CCC(=O)NC5=O)noc4c3)cn2)cc1] |
| 340. | 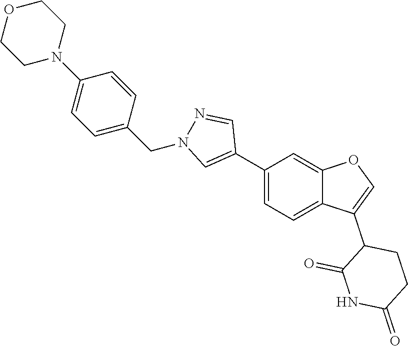 [SMILES: O=C1CCC(c2coc3cc(-c4cnn(Cc5ccc(N6CCOCC6)cc5)c4)ccc23)C(=O)N1] |
| 183. | 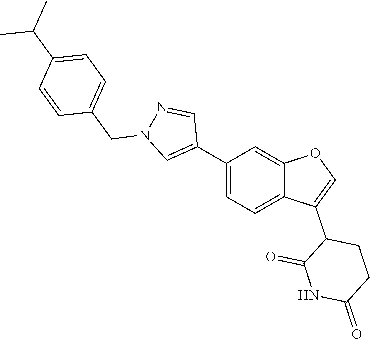 [SMILES: CC(C)c1ccc(Cn2cc(-c3ccc4c(C5CCC(=O)NC5=O)coc4c3)cn2)cc1] |
| 232. | 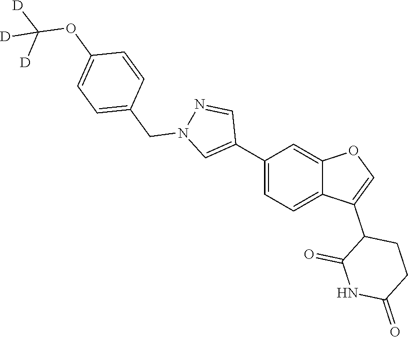 [SMILES: [2H]C([2H])([2H])Oc1ccc(Cn2cc(-c3ccc4c(C5CCC(=O)NC5=O)coc4c3)cn2)cc1] |
| 233. | 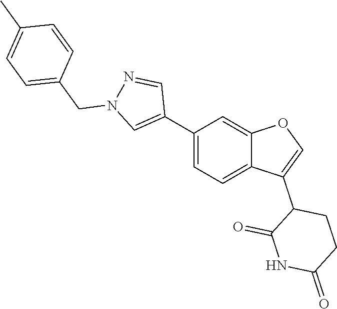 [SMILES: Cc1ccc(Cn2cc(-c3ccc4c(C5CCC(=O)NC5=O)coc4c3)cn2)cc1] |
| 286. | 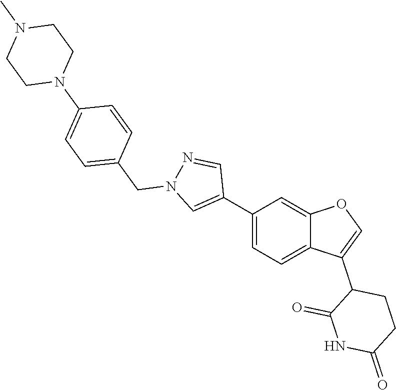 [SMILES: CN1CCN(c2ccc(Cn3cc(-c4ccc5c(C6CCC(=O)NC6=O)coc5c4)cn3)cc2)CC1] |
| 265. | 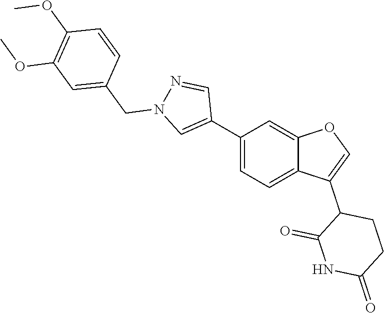 [SMILES: COc1ccc(Cn2cc(-c3ccc4c(C5CCC(=O)NC5=O)coc4c3)cn2)cc1OC] |
| 240. | 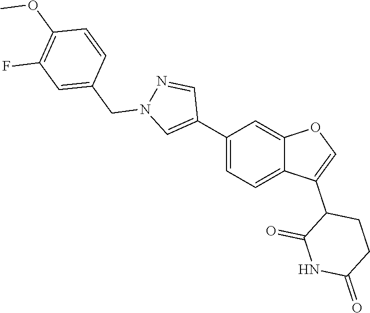 [SMILES: COc1ccc(Cn2cc(-c3ccc4c(C5CCC(=O)NC5=O)coc4c3)cn2)cc1F] |
| 166. | 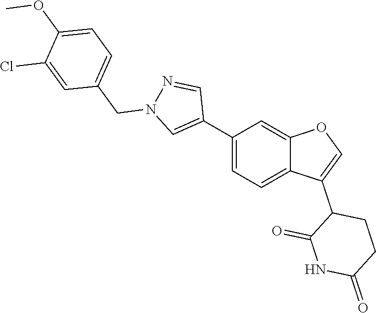 [SMILES: COc1ccc(Cn2cc(-c3ccc4c(C5CCC(=O)NC5=O)coc4c3)cn2)cc1Cl] |
| 343. | 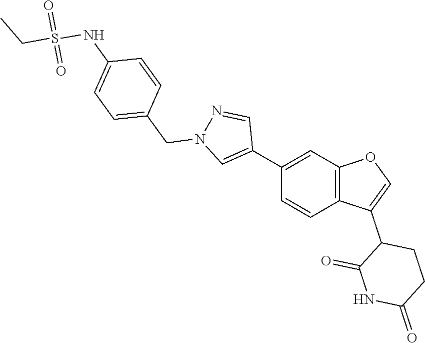 [SMILES: CCS(=O)(=O)Nc1ccc(Cn2cc(-c3ccc4c(C5CCC(=O)NC5=O)coc4c3)cn2)cc1] |
| 134. | 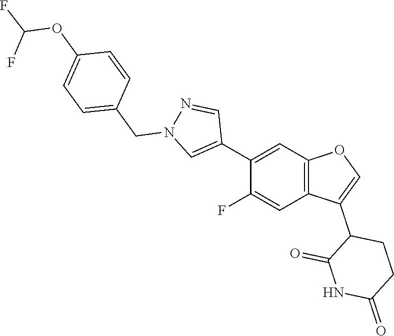 [SMILES: O=C1CCC(c2coc3cc(-c4cnn(Cc5ccc(OC(F)F)cc5)c4)c(F)cc23)C(=O)N1] |
| 187. | 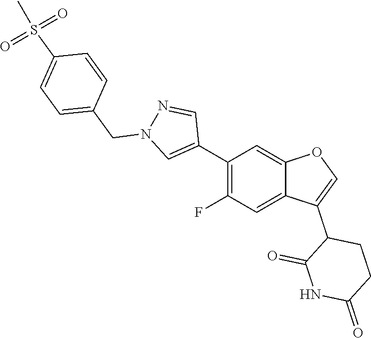 [SMILES: CS(=O)(=O)c1ccc(Cn2cc(-c3cc4occ(C5CCC(=O)NC5=O)c4cc3F)cn2)cc1] |
| 230. | 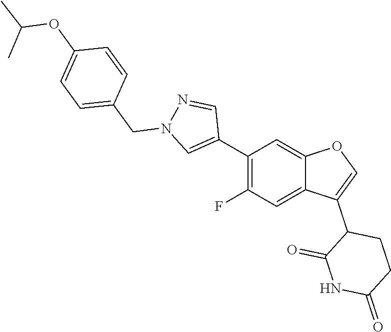 [SMILES: CC(C)Oc1ccc(Cn2cc(-c3cc4occ(C5CCC(=O)NC5=O)c4cc3F)cn2)cc1] |
| 199. | 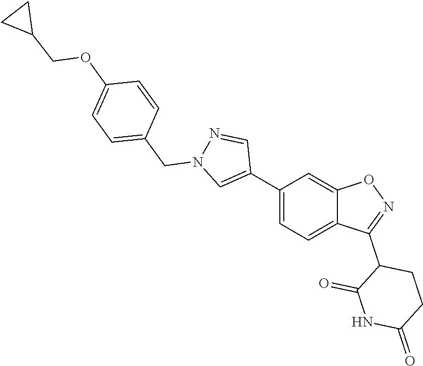 [SMILES: O=C1CCC(c2noc3cc(-c4cnn(Cc5ccc(OCC6CC6)cc5)c4)ccc23)C(=O)N1] |
| 342. | 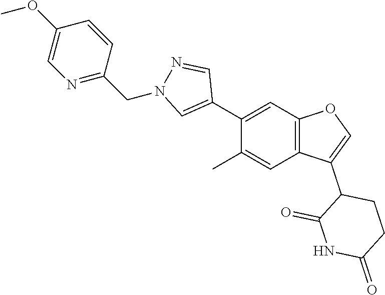 [SMILES: COc1ccc(Cn2cc(-c3cc4occ(C5CCC(=O)NC5=O)c4cc3C)cn2)nc1] |
| 350. | 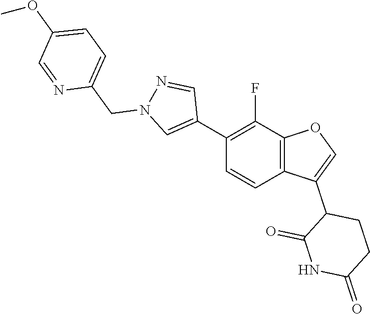 [SMILES: COc1ccc(Cn2cc(-c3ccc4c(C5CCC(=O)NC5=O)coc4c3F)cn2)nc1] |
| 351. | 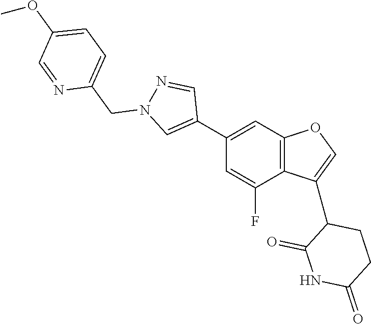 [SMILES: COc1ccc(Cn2cc(-c3cc(F)c4c(C5CCC(=O)NC5=O)coc4c3)cn2)nc1] |
| 339. | 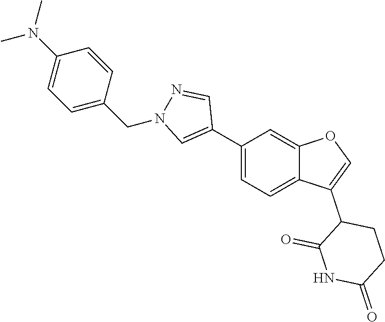 [SMILES: CN(C)c1ccc(Cn2cc(-c3ccc4c(C5CCC(=O)NC5=O)coc4c3)cn2)cc1] |
| 352. | 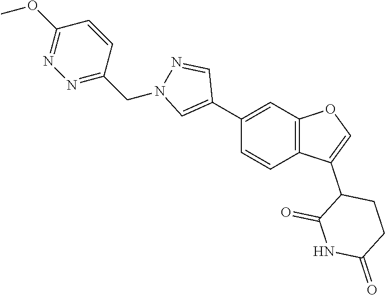 [SMILES: COc1ccc(Cn2cc(-c3ccc4c(C5CCC(=O)NC5=O)coc4c3)cn2)nn1] |
| 347. | 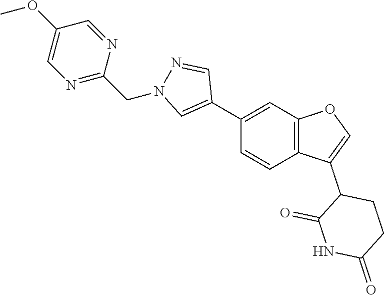 [SMILES: COc1cnc(Cn2cc(-c3ccc4c(C5CCC(=O)NC5=O)coc4c3)cn2)nc1] |
| 378. | 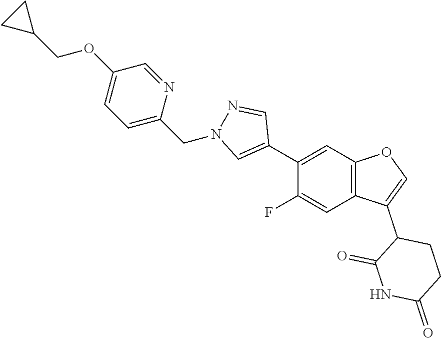 [SMILES: O=C1CCC(c2coc3cc(-c4cnn(Cc5ccc(OCC6CC6)cn5)c4)c(F)cc23)C(=O)N1] |
| 385. | 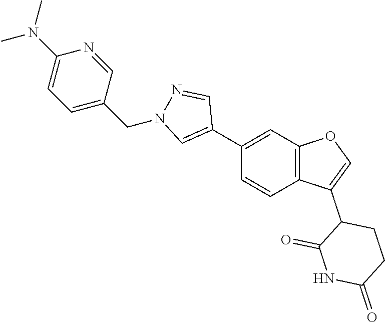 [SMILES: CN(C)c1ccc(Cn2cc(-c3ccc4c(C5CCC(=O)NC5=O)coc4c3)cn2)cn1] |
| 394. | 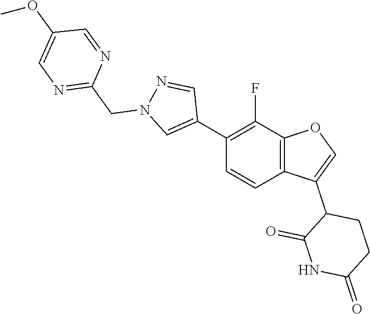 [SMILES: COc1cnc(Cn2cc(-c3ccc4c(C5CCC(=O)NC5=O)coc4c3F)cn2)nc1] |
| 402. | 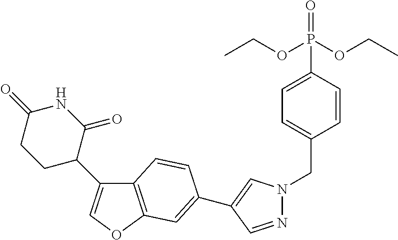 [SMILES: CCOP(=O)(OCC)c1ccc(Cn2cc(-c3ccc4c(C5CCC(=O)NC5=O)coc4c3)cn2)cc1] |
| 403. | 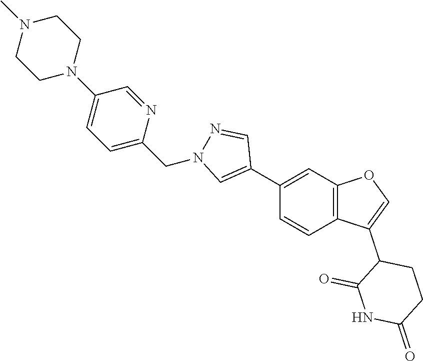 [SMILES: CN1CCN(c2ccc(Cn3cc(-c4ccc5c(C6CCC(=O)NC6=O)coc5c4)cn3)nc2)CC1] |
| 420. | 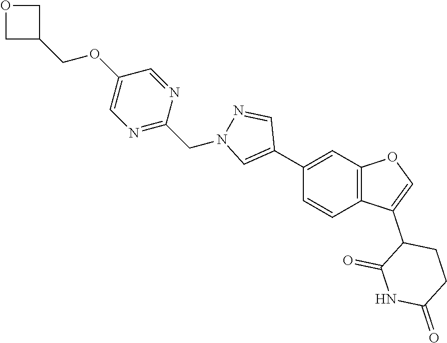 [SMILES: O=C1CCC(c2coc3cc(-c4cnn(Cc5ncc(OCC6COC6)cn5)c4)ccc23)C(=O)N1] |
| 424. | 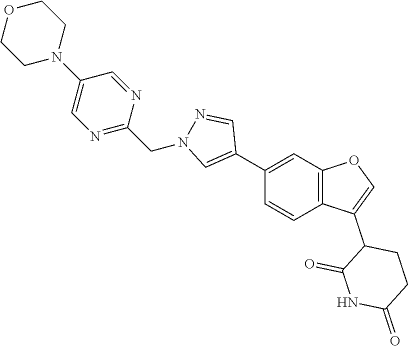 [SMILES: O=C1CCC(c2coc3cc(-c4cnn(Cc5ncc(N6CCOCC6)cn5)c4)ccc23)C(=O)N1] |
| 426. | 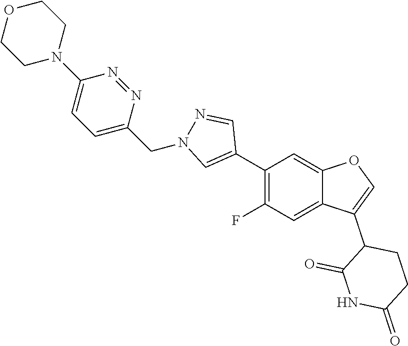 [SMILES: O=C1CCC(c2coc3cc(-c4cnn(Cc5ccc(N6CCOCC6)nn5)c4)c(F)cc23)C(=O)N1] |
| 428. | 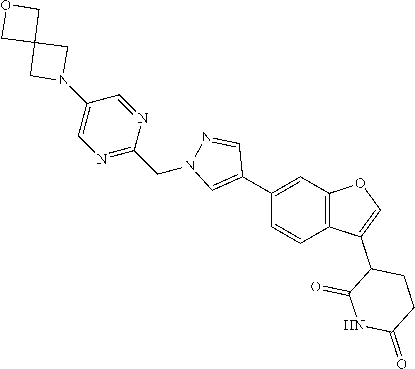 [SMILES: O=C1CCC(c2coc3cc(-c4cnn(Cc5ncc(N6CC7(COC7)C6)cn5)c4)ccc23)C(=O)N1] |
| 429. | 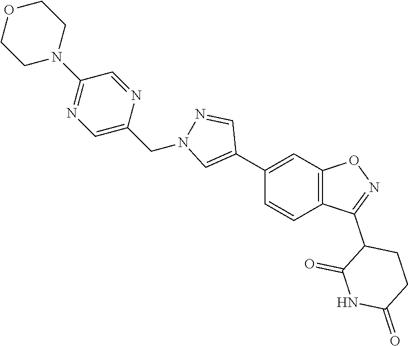 [SMILES: O=C1CCC(c2noc3cc(-c4cnn(Cc5cnc(N6CCOCC6)cn5)c4)ccc23)C(=O)N1] |
| 431. | 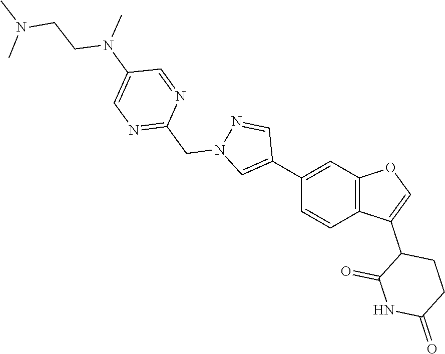 [SMILES: CN(C)CCN(C)c1cnc(Cn2cc(-c3ccc4c(C5CCC(=O)NC5=O)coc4c3)cn2)nc1] |
| 441. | 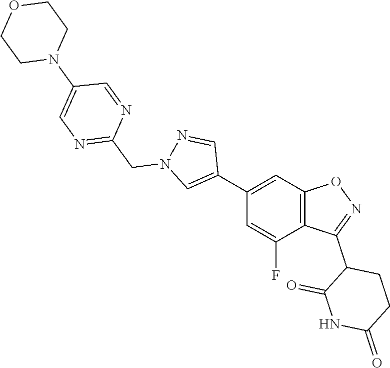 [SMILES: O=C1CCC(c2noc3cc(-c4cnn(Cc5ncc(N6CCOCC6)cn5)c4)cc(F)c23)C(=O)N1] |
| 445. | 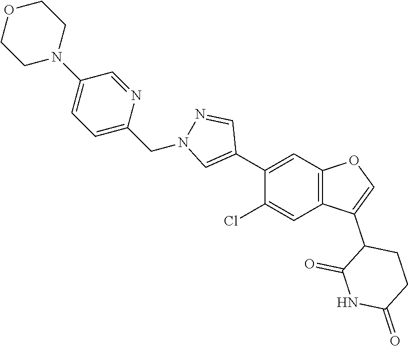 [SMILES: O=C1CCC(c2coc3cc(-c4cnn(Cc5ccc(N6CCOCC6)cn5)c4)c(Cl)cc23)C(=O)N1] |
| 460. | 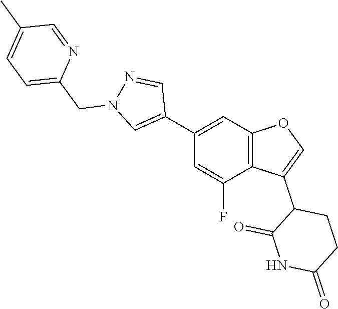 [SMILES: Cc1ccc(Cn2cc(-c3cc(F)c4c(C5CCC(=O)NC5=O)coc4c3)cn2)nc1] |
| 464. | 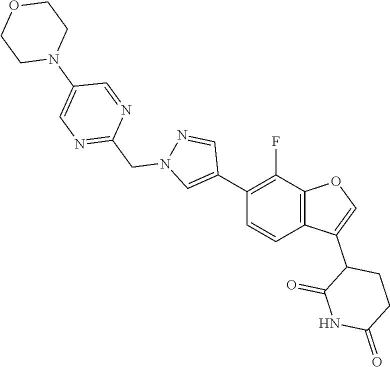 [SMILES: O=C1CCC(c2coc3c(F)c(-c4cnn(Cc5ncc(N6CCOCC6)cn5)c4)ccc23)C(=O)N1] |
| 469. | 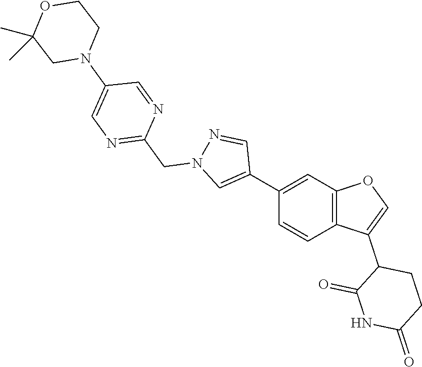 [SMILES: CC1(C)CN(c2cnc(Cn3cc(-c4ccc5c(C6CCC(=O)NC6=O)coc5c4)cn3)nc2)CCO1] |
| 475. | 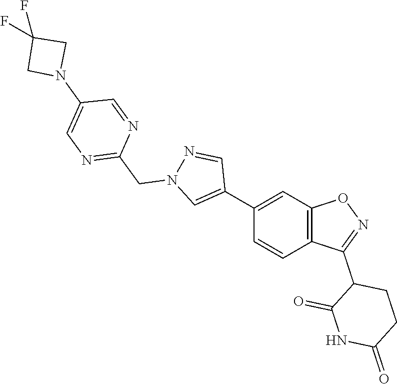 [SMILES: O=C1CCC(c2noc3cc(-c4cnn(Cc5ncc(N6CC(F)(F)C6)cn5)c4)ccc23)C(=O)N1] |
| 486. | 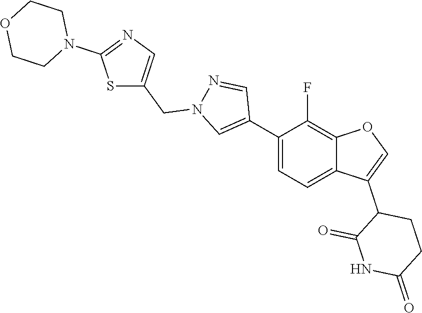 [SMILES: O=C1CCC(c2coc3c(F)c(-c4cnn(Cc5cnc(N6CCOCC6)s5)c4)ccc23)C(=O)N1] |
| 487. | 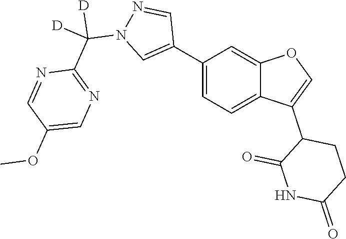 [SMILES: [2H]C([2H])(c1ncc(OC)cn1)n1cc(-c2ccc3c(C4CCC(=O)NC4=O)coc3c2)cn1] |
| 489. | 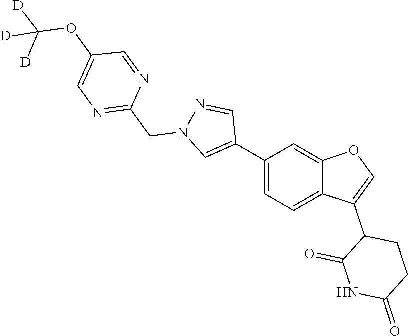 [SMILES: [2H]C([2H])([2H])Oc1cnc(Cn2cc(-c3ccc4c(C5CCC(=O)NC5=O)coc4c3)cn2)nc1] |
| 491. | 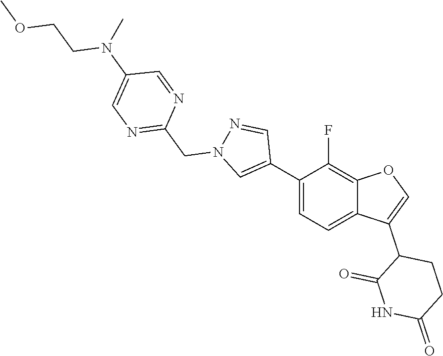 [SMILES: COCCN(C)c1cnc(Cn2cc(-c3ccc4c(C5CCC(=O)NC5=O)coc4c3F)cn2)nc1] |
| 496. | 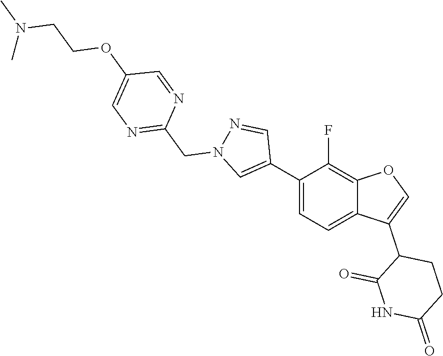 [SMILES: CN(C)CCOc1cnc(Cn2cc(-c3ccc4c(C5CCC(=O)NC5=O)coc4c3F)cn2)nc1] |

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### Definitions

The articles “a” and “an” are used in this application to refer to one or more than one (i.e., at least one) of the grammatical object of the article. By way of example, “an element” means one element or more than one element.

The term “and/or” is used in this application to mean either “and” or “or” unless indicated otherwise.

The application also includes pharmaceutical compositions comprising an effective amount of a compound of the present application (e.g., a compound of any of the formulae or any individual compounds disclosed herein) and a pharmaceutically acceptable carrier.

The term “alkyl,” as used herein, refers to saturated, straight or branched-chain hydrocarbon radicals containing, in some embodiments, between one and six carbon atoms. Examples of C₁-C₈ alkyl radicals include, but are not limited to, methyl, ethyl, propyl, isopropyl, n-butyl, tert-butyl, neopentyl, n-hexyl, n-heptyl, and n-octyl radicals. Examples of C₁-C₆ alkyl radicals include, but are not limited to, methyl, ethyl, propyl, isopropyl, n-butyl, tert-butyl, neopentyl, and n-hexyl radicals.

The term “alkenyl,” as used herein, denotes a monovalent group derived from a hydrocarbon moiety containing, in certain embodiments, from two to six carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl and the like.

The term “alkoxy” refers to an—O-alkyl radical.

The terms “hal,” “halo,” and “halogen,” as used herein, refer to an atom selected from fluorine, chlorine, bromine and iodine.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, indenyl and the like.

The term “aralkyl,” as used herein, refers to an alkyl residue attached to an aryl ring. Examples include, but are not limited to, benzyl, phenethyl and the like.

The term “cycloalkyl” or “carbocyclyl,” as used herein, denotes a monovalent group derived from a monocyclic or polycyclic saturated or partially unsaturated carbocyclic ring compound (fused, bridged, or spiro rings). Examples of C₃-C₈ cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl. Also contemplated is a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O, and N; zero, one, or two ring atoms are additional heteroatoms independently selected from S, O, and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl” as used herein, refers to an alkyl residue attached to a heteroaryl ring. Examples include, but are not limited to, pyridinylmethyl, pyrimidinylethyl and the like.

The term “heterocyclyl” or “heterocycloalkyl,” as used herein, refers to a saturated or unsaturated non-aromatic 3-, 4-, 5-, 6-, 7-, or 8-membered monocyclic, 7-, 8-, 9-, 10-, 11-, or 12-membered bicyclic (fused, bridged, or spiro rings), or 11-, 12, 13, or 14-membered tricyclic ring system (fused, bridged, or spiro rings), where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, and (iv) the nitrogen heteroatom may optionally be quaternized. Representative heterocycloalkyl groups include, but are not limited to, [1,3]dioxolanyl, pyrrolidinyl, pyrazolidinyl, pyrazolinyl, imidazolinyl, imidazolidinyl, piperidinyl, pipcrazinyl, 2-pyridone, oxazolidinyl, isoxazolidinyl, morpholinyl, tetrahydropyranyl, thiazolidinyl, isothiazolidinyl, tetrahydrofuryl, dioxanyl, oxetanyl, azetidinyl, thietanyl, oxiranyl, aziridinyl, thiiranyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 2-azaspiro[3.3]heptan-5-amine, 1-azaspiro[3.3]heptan-5-amine, 1-oxa-6-azaspiro[3.3]heptan-3-amine, 2-azaspiro[3.3]heptan-6-amine, 1-azaspiro[3.3]heptan-6-amine, 6-azaspiro[3.4]octan-2-amine, 5-azaspiro[3.4]octan-2-amine, 6-azaspiro[3.4]octan-1-amine, 5-azaspiro[3.4]octan-1-amine, 5-oxa-2-azaspiro[3.4]octan-7-amine, 7-amino-5-thia-2-azaspiro[3.4]octane 5,5-dioxide, 5-oxa-2-azaspiro[3.4]octan-8-amine, 8-amino-5-thia-2-azaspiro[3.4]octane 5,5-dioxide, and the like.

The term “alkylamino” refers to a group having the structure, e.g., NH(C₁-C₆ alkyl), where C₁-C₆ alkyl is as previously defined.

The term “dialkylamino” refers to a group having the structure, e.g., N(C₁-C₆ alkyl)₂, where C₁-C₆ alkyl is as previously defined.

In accordance with the application, any of the aryls, substituted aryls, heteroaryls and substituted heteroaryls described herein, can be any aromatic group. Aromatic groups can be substituted or unsubstituted.

As described herein, compounds of the application may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocyclyl,” and any other optionally substituted group as used herein, refer to groups that are substituted or unsubstituted by independent replacement of one, two, or three or more of the hydrogen atoms thereon with substituents including, but not limited to:
—F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —C(O)NH₂, —C(O)NH—C₁-C₁₂-alkyl, —C(O)NH—C₂-C₁₂-alkenyl, —C(O)NH—C₂-C₁₂-alkenyl, —C(O)NH—C₃-C₁₂-cycloalkyl, —C(O)NH-aryl, —C(O)NH-heteroaryl, —C(O)NH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OC(O)NH₂, —OC(O)NH—C₁-C₁₂-alkyl, —OC(O)NH—C₂-C₁₂-alkenyl, —OC(O)NH—C₂-C₁₂-alkenyl, —OC(O)NH—C₃-C₁₂-cycloalkyl, —OC(O)NH-aryl, —OC(O)NH-heteroaryl, —OC(O)NH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S) NH₂, —NHC(S) NH—C₁-C₁₂-alkyl, —NHC(S) NH—C₂-C₁₂-alkenyl, —NHC(S) NH—C₂-C₁₂-alkenyl, —NHC(S) NH—C₃-C₁₂-cycloalkyl, —NHC(S) NH-aryl, —NHC(S) NH-heteroaryl, —NHC(S) NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)-C₃—C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO—NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The compounds of the present application may form salts which are also within the scope of this application. Reference to a compound herein is understood to include reference to salts thereof, unless otherwise indicated.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, malate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The compounds of the present application, for example, including the pharmaceutically acceptable salts, solvates, prodrugs, stereoisomers, or tautomers of the compounds, can exist in a solvated form with other solvent molecules or in an unsolvated form.

“Solvate” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastercomeric forms, are contemplated within the scope of this application, as are positional isomers (such as, for example, 4-pyridyl and 3-pyridyl). Individual stereoisomers of the compound of the application may, for example, be substantially free of other isomers, or may be admixed, for example, as racemates or with all other, or other selected, stereoisomers. The chiral centers of the present application can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of the present application (e.g., a compound of any of the formulae or any individual compounds disclosed herein) may have one or more asymmetric carbon atom and may occur as racemates, racemic mixtures or as individual enantiomers or diastereomers.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

The compounds of the application may contain asymmetric or chiral centers, and, therefore, exist in different stereoisomeric forms. It is intended that all stereoisomeric forms of the compounds of the application as well as mixtures thereof, including racemic mixtures, form part of the present application. In addition, the present application embraces all geometric and positional isomers. For example, if a compound of the application incorporates a double bond or a fused ring, both the cis- and trans-forms, as well as mixtures, are embraced within the scope of the application. Each compound herein disclosed includes all the enantiomers that conform to the general structure of the compound. The compound may be in a racemic or enantiomerically pure form, or any other form in terms of stereochemistry. The assay results may reflect the data collected for the racemic form, the enantiomerically pure form, or any other form in terms of stereochemistry.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastercomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

In another embodiment of the application, the compound of the present application (e.g., a compound of any of the formulae or any individual compounds disclosed herein) is an enantiomer. In some embodiments the compound is the(S)-enantiomer. In other embodiments the compound is the (R)-enantiomer. In yet other embodiments, the compounds of the present application (e.g., a compound of any of the formulae or any individual compounds disclosed herein) may be (+) or (−) enantiomers. The compound may contain more than one stereocenter.

In another embodiment of the application, the compounds of the present application (e.g., a compound of any of the formulae or any individual compounds disclosed herein) are diastereomers. In some embodiments, the compounds are the syn diastereomer. In other embodiments, the compounds are the anti diastereomer.

Diastercomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastercomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

It is also possible that the compounds of the application may exist in different tautomeric forms, and all such forms are embraced within the scope of the application. Also, for example, all keto-enol and imine-enamine forms of the compounds are included in the application.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-imine.

The compounds of the present application can be converted to N-oxides by treatment with an oxidizing agent (e.g., 3-chloroperoxybenzoic acid (m-CPBA) and/or hydrogen peroxides) to afford other compounds of the present application. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present application can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compounds as shown and its N-hydroxy (i.e., N—OH) and N-alkoxy (i.e., N—OR, wherein R is substituted or unsubstituted C₁—C 6 alkyl, C₁-C₆ alkenyl, C₁-C₆ alkynyl, 3-14-membered carbocycle or 3-14-membered heterocycle) derivatives.

The term “prodrug,” as used in this application, means a compound which is convertible in vivo by metabolic means (e.g., by hydrolysis) to a disclosed compound.

Since prodrugs are known to enhance numerous desirable qualities of pharmaceuticals (e.g., solubility, bioavailability, manufacturing, etc.) the compounds of the present application (e.g., a compound of any of the formulae or any individual compounds disclosed herein), or pharmaceutically acceptable salts, solvates, prodrugs, stereoisomers, or tautomers thereof can be delivered in prodrug form. Thus, the present application is intended to cover prodrugs of a compound of the present application (e.g., a compound of any of the formulae or any individual compounds disclosed herein), or a pharmaceutically acceptable salt, solvate, prodrug, stereoisomer, or tautomer thereof, methods of delivering the same and compositions containing the same. “Prodrugs” are intended to include any covalently bonded carriers that release an active parent drug of the present application in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the application wherein a hydroxyl or amino, group is bonded to any group that, when the prodrug of the present application is administered to a mammalian subject, it cleaves to form a free hydroxyl or free amino group, respectively. Examples of prodrugs include, but are not limited to, acetate, formate, and benzoate derivatives of alcohol and amine functional groups in the compounds of each of the formulae described herein or a pharmaceutically acceptable salt, solvate, prodrug, stereoisomer, or tautomer thereof.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate.

As used herein, the term “analog” refers to a compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

The application also comprehends isotopically-labeled compounds, which are identical to those recited in the each of the formulae described herein, but for the fact that one or more atoms are replaced by an atom having an atomic mass or mass number different from the atomic mass or mass number most commonly found in nature. Examples of isotopes that can be incorporated into compounds of the application include isotopes of hydrogen, carbon, nitrogen, fluorine, such as ³H, ¹¹C, ¹⁴C, ²H and ¹⁸F.

Compounds of the present application (e.g., a compound of any of the formulae or any individual compounds disclosed herein), or pharmaceutically acceptable salts, solvates, prodrugs, stereoisomers, or tautomers thereof, that contain the aforementioned isotopes and/or other isotopes of other atoms are within the scope of the present application. Isotopically-labeled compounds of the present application, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are useful for their case of preparation and detectability. ¹¹C and ¹⁸F isotopes are useful in PET (positron emission tomography). PET is useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances, isotopically labeled compounds of the present application (e.g., a compound of any of the formulae or any individual compounds disclosed herein), or pharmaceutically acceptable salts, solvates, prodrugs, stereoisomers, or tautomers thereof, can generally be prepared by carrying out the procedures disclosed in the Schemes and/or in the Examples described herein, by substituting a readily available isotopically labeled reagent for a non-isotopically labeled reagent. In one embodiment, the compound of the present application (e.g., a compound of any of the formulae or any individual compounds disclosed herein) or pharmaceutically acceptable salts, solvates, prodrugs, stereoisomers, or tautomers thereof, is not isotopically labelled.

The term “administer”, “administering”, or “administration” as used in this application refers to either directly administering a disclosed compound or pharmaceutically acceptable salt of the disclosed compound or a composition to a subject, or administering a prodrug, derivative or analog of the compound or pharmaceutically acceptable salt of the compound or a composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The compounds of the present application, or a pharmaceutically acceptable salt, solvate, prodrug, stereoisomer, or tautomer thereof, can also be used to prevent a disease, condition or disorder. As used herein, “preventing” or “prevent” describes reducing or eliminating the onset of the symptoms or complications of the disease, condition or disorder.

The term “disorder” is used in this application to mean, and is used interchangeably with, the terms disease, condition, or illness, unless otherwise indicated.

As used herein, the term “HuR-mediated” diseases or disorders or diseases or disorders “in which HuR plays a role” or “associated with HuR” means any disease or other deleterious condition in which HuR, or a mutant thereof, is known to play a role. Accordingly, another embodiment of the present application relates to treating or lessening the severity of one or more diseases in which HuR, or a mutant thereof, is known to play a role. Specifically, the present application relates to treating or lessening the severity of a disease or condition selected from a cell proliferative disease or disorder, such as cancer (e.g.,, bladder cancer, brain cancer, breast cancer, colorectal cancer, cervical cancer, gastrointestinal cancer, genitourinary cancer, glioma, head and neck cancer, lung cancer, ovarian cancer, pancreatic cancer, prostate cancer, renal cancer, skin cancer, or testicular cancer), by using compounds of the present application (e.g., a compound of any of the formulae or any individual compounds disclosed herein), or pharmaceutically acceptable salts, solvates, prodrugs, stereoisomers, or tautomers thereof, or a composition according to the present application.

### Methods for Preparing the Compounds

The compounds of the present application may be made by a variety of methods, including standard chemistry. Suitable synthetic routes are depicted in the Schemes given below.

The compounds of the present application (e.g., a compound of any of the formulae or any individual compounds disclosed herein) may be prepared by methods known in the art of organic synthesis as set forth in part by the following synthetic schemes. In the scheme described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of the compounds of the present application.

Those skilled in the art will recognize if a stereocenter exists in the compounds of the present application (e.g., a compound of any of the formulae or any individual compounds disclosed herein). Accordingly, the present application includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

The compounds of the present application can be prepared in a number of ways well known to those skilled in the art of organic synthesis. By way of example, the compounds of the present application can be synthesized using the methods described below, together with synthetic methods known in the art of synthetic organic chemistry, or variations thereon as appreciated by those skilled in the art. Preferred methods include but are not limited to those methods described below. The compounds of the present application (i.e., a compound of the present application (e.g., a compound of any of the formulae or any individual compounds disclosed herein)) can be synthesized by following the steps outlined in the examples, schemes, procedures, and/or synthesis described herein (e.g., Examples). Starting materials are either commercially available or made by known procedures in the reported literature or as illustrated.

A mixture of enantiomers, diastereomers, cis/trans isomers resulting from the processes described above can be separated into their single components by chiral salt technique, chromatography using normal phase, reverse phase or chiral column, depending on the nature of the separation.

The present application relates to methods of synthesizing a compound of the application or a pharmaceutically acceptable salt, solvate, prodrug, stereoisomer, or tautomer thereof. The schemes and description below depict general routes for the preparation of a compound of the application. For example, compounds of the present application can be synthesized by following the steps outlined in Scheme 1 or 2. Starting materials are either commercially available or made by known procedures in the reported literature or as illustrated.

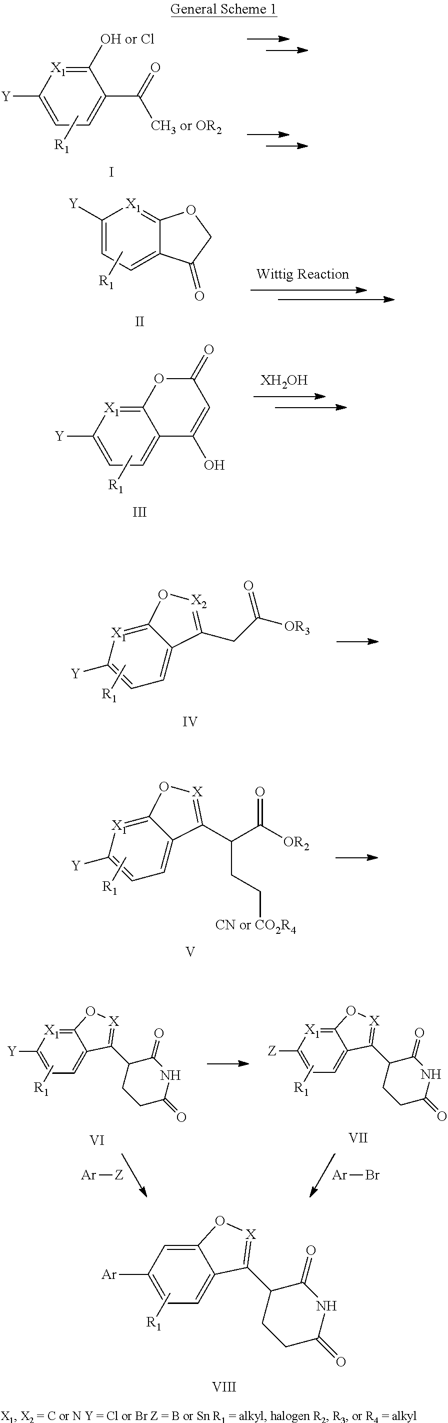

Accordingly General Scheme 1, a compound of 1 is transformed through several steps to intermediates such as 6-halo-benzofuran-3-one or its azo counterpart II or 7-halo substituted-4-hydroxy-chromen-2-one or its azo counterpart III, which is subsequently converted to substituted acetate IV. Key intermediates VI is obtained through alkylation of IV, with or without the isolation of V. Target compounds VIII are obtained when Intermediate VI were coupled with organoboron or tin reagents in the presence of properly selected transition metal catalysts. Alternatively, Intermediated VI can be transformed first to their corresponding organoboron or tin reagents VII before coupling with properly substituted ArBr to deliver the target compounds VIII.

Additional schemes are also available, such as General Scheme 2.

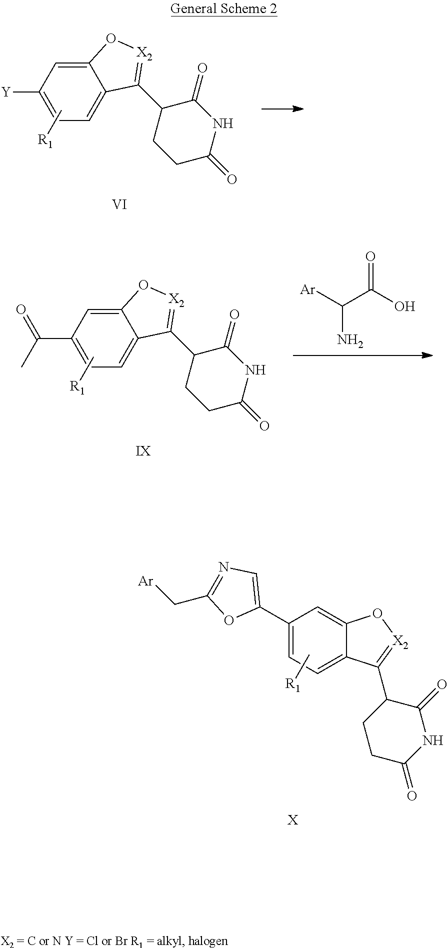

A mixture of enantiomers, diastereomers, cis/trans isomers resulting from the processes described above can be separated into their single components by chiral salt technique, chromatography using normal phase, reverse phase or chiral column, depending on the nature of the separation.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz.
Abbreviations frequently used in the art or herein are:
DIEA N,N-diisopropylethylamine
DMF N,N-dimethylformamide
DMA N,N-dimethylacetamide
DMSO dimethylsulfoxide
DEAD DiethylAzodicarboxylate
EA ethyl acetate
IPA iso-propyl alcohol
IPE di-isopropyl ether
MeCN acetonitrile
THF tetrahydrofuran
m-CPBA 3-chlorobenzenecarboperoxoic acid
DCM dichloromethane
LC/MS liquid chromatography-mass spectrometry
MeOH methanol
MS mass spectrometry
PE petroleum ether
NMP N-methyl pyrrolidinone
NMR nuclear magnetic resonance
Ppm parts per million
TEA triethylamine
NBS 1-bromopyrrolidine-2,5-dione
AIBN 2,2-Azobis(2-methylpropionitrile)
DBAD Di-tert-butyl diazene-1,2-dicarboxylate
XPhos Pd G3 Methanesulfonato (2-dicyclohexylphosphino-2,4,6-tri-i-propyl-1,1-biphenyl) (2-amino-1,1-biphenyl-2-yl) palladium (II)
TBUP Tributylphosphine
TMAD N,N,N,N-Tetramethylazodicarboxamide

### Biological Assays

The biological activities of the compounds of the present application can be assessed with methods and assays known in the art. Exemplary methods are described in the Examples.

### Methods of Using the Compounds

The compounds of the present application are useful for degrading HuR. As such, the compounds of the present application are useful for the treatment or prevention of a disease or disorder associated with HuR.

In one aspect of the application relates to a method of treating, preventing, inhibiting, or eliminating a disease or disorder associated with HuR (e.g., HuR overexpression or dysregulation). The method comprises administering to a subject in need of a treatment for diseases or disorders associated with HuR an effective amount a compound of the present application (e.g., a compound of any of the formulae or any individual compounds disclosed herein) or a pharmaceutically acceptable salt, solvate, prodrug, stereoisomer, or tautomer thereof or a pharmaceutical composition of a compound of the present application (e.g., a compound of any of the formulae or any individual compounds disclosed herein).

Another aspect of the application relates to a compound of the present application (e.g., a compound of any of the formulae or any individual compounds disclosed herein), or a pharmaceutically acceptable salt, solvate, prodrug, stereoisomer, or tautomer thereof, for use in a method of treating or preventing a HuR mediated disease or disorder.

In another aspect, the present application relates to a pharmaceutical composition of a compound of the present application (e.g., a compound of any of the formulae or any individual compounds disclosed herein), or a pharmaceutically acceptable salt, solvate, prodrug, stereoisomer, or tautomer thereof, for use in a method of treating or preventing a HuR mediated disease or disorder.

Another aspect of the application relates to the use of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for treating or preventing a HuR mediated disease or disorder.

In another aspect, the present application relates to the use of a pharmaceutical composition of a compound of the present application (e.g., a compound of any of the formulae or any individual compounds disclosed herein), or a pharmaceutically acceptable salt, solvate, prodrug, stereoisomer, or tautomer thereof, in the manufacture of a medicament for treating or preventing a HuR mediated disease or disorder.

The disclosed compound of the application can be administered in effective amounts to treat or prevent a disorder and/or prevent the development thereof in subjects.

In some embodiments, the disease or disorder is a disease or disorder with HuR overexpression or dysregulation.

In some embodiments, the disease or disorder with HuR overexpression or dysregulation is a cancer.

In some embodiments, the cancer is a adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, basal cell carcinoma (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary Syndrome, endometrial cancer, esophageal cancer, extracranial germ cell tumor, extragonadal germ cell tumor, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, ocular cancer, islet cell tumors (endocrine pancreas), Kaposi Sarcoma, kidney cancer, renal cancer, laryngeal cancer, acute lymphoblastic leukemia, acute myeloid leukemia, hairy cell leukemia, lip and oral cavity cancer, lung cancer, non-small cell lung cancer, small cell lung cancer, non-Hodgkin lymphoma, primary central nervous system lymphoma, Waldenstroem macroglobulinemia, melanoma, Merkel cell carcinoma, mesothelioma malignant, mesothelioma, metastatic squamous neck cancer, mouth cancer, cancer of the tongue, multiple endocrine neoplasia syndrome, myelodysplastic syndromes, myelodysplastic/myeloproliferative diseases, multiple myeloma, nasopharyngeal cancer, neuroblastoma, oral cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm, pleuropulmonary blastoma, prostate cancer, rectal cancer, renal pelvis and ureter, transitional cell cancer, rhabdomyosarcoma, salivary gland cancer, Ewing family of sarcoma tumors, soft tissue sarcoma, synovial sarcoma, uterine cancer, uterine sarcoma, skin cancer (non-melanoma), Merkel cell skin carcinoma, small intestine cancer, squamous cell carcinoma, testicular cancer, throat cancer, thymoma carcinoma, thymic carcinoma, thyroid cancer, transitional cell cancer of the renal pelvis and ureter and other urinary organs, gestational trophoblastic tumor, urethral cancer, endometrial uterine cancer, uterine corpus cancer, vaginal cancer, vulvar cancer, Wilm's Tumor, lymphoma, or diffuse large B-cell lymphoma (DLBCL).

In some embodiments, the cancer is a bladder cancer, brain cancer, breast cancer, colorectal cancer, cervical cancer, gastrointestinal cancer, genitourinary cancer, glioma, head and neck cancer, lung cancer, ovarian cancer, pancreatic cancer, prostate cancer, renal cancer, skin cancer, or testicular cancer.

The compound of the application can be administered in therapeutically effective amounts in a combinational therapy with one or more therapeutic agents (pharmaceutical combinations) or modalities, e.g., non-drug therapies. For example, synergistic effects can occur with other anti-proliferative, anti-cancer, immunomodulatory or anti-inflammatory substances. In some embodiments, a compound of the present application (e.g., a compound of any of the formulae or any individual compounds disclosed herein) is administered in combination with an additional therapeutic agent selected from an anti-inflammatory agent, an immunomodulatory agent, a chemotherapeutic agent, an agent for treating cardiovascular disease, an agent for treating liver disease, an agent for treating lung disease, an agent for treating kidney disease, an agent for treating ocular disease, an agent for treating skin disease, an anti-viral agent, an agent for treating blood disorders, an agent for treating diabetes, and an agent for treating immunodeficiency disorders. Where the compound of the application is administered in conjunction with other therapies, dosages of the co-administered compounds will of course vary depending on the type of co-drug employed, on the specific drug employed, on the condition being treated and so forth.

Combination therapy includes the administration of the subject compound in further combination with other biologically active ingredients (such as, but not limited to, an anti-inflammatory agent, an immunomodulatory agent, chemotherapeutic agent, an agent for treating cardiovascular disease, an agent for treating liver disease, an anti-viral agent, an agent for treating blood disorders, an agent for treating diabetes, an agent for treating immunodeficiency disorders, and an agent for treating pain) and non-drug therapies (such as, but not limited to, surgery or radiation treatment). For instance, the compound of the application can be used in combination with other pharmaceutically active compounds, preferably compounds that are able to enhance the effect of the compound of the application. The compound of the application can be administered simultaneously (as a single preparation or separate preparation) or sequentially to the other drug therapy or treatment modality. In general, a combination therapy envisions administration of two or more drugs during a single cycle or course of therapy.

### Pharmaceutical Compositions

The present application also provides pharmaceutical compositions comprising a compound of the present application (e.g., a compound of any of the formulae or any individual compounds disclosed herein), or a pharmaceutically acceptable salt, solvate, prodrug, stereoisomer, or tautomer thereof, in combination with at least one pharmaceutically acceptable excipient or carrier.

A “pharmaceutical composition” is a formulation containing the compound of the present application in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or a pharmaceutically acceptable salt, solvate, prodrug, stereoisomer, or tautomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical us. A “pharmaceutically acceptable excipient” as used in the specification and claims includes both one and more than one such excipient.

A pharmaceutical compositions of the application are formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. The dose chosen should be sufficient to constitute effective treatment but not as high as to cause unacceptable side effects. The state of the disease condition and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or modulatory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In one embodiment, the disease or disorder is a disease or disorder described herein.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compound (i.e., a compound of the present application (e.g., a compound of any of the formulae or any individual compounds disclosed herein)) of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compound into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compound is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The active compound can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for case of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Dosages can range from about 0.01 mg/kg per day to about 5000 mg/kg per day. An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

The pharmaceutical compositions can be included in a container, pack, or dispenser together with instructions for administration.

As used herein, “pharmaceutically acceptable salts” refer to derivatives of the compound of the present application wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amino acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present application also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compound of the present application can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compound of the present application can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compound of the present application can be delivered in prodrug form. Thus, the present application is intended to cover prodrugs of the presently claimed compound, methods of delivering the same and compositions containing the same. “Prodrugs” are intended to include any covalently bonded carriers that release an active parent drug of the present application in vivo when such prodrug is administered to a subject. Prodrugs in the present application are prepared by modifying functional groups present in the compound in such a way that the modifications are cleaved, either in routine manipulation or in vivo, to the parent compound. Prodrugs include the compound of the present application wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in the compound of the application, and the like, See Bundegaard, H., Design of Prodrugs, p1-92, Elsevier, New York-Oxford (1985).

The compound, or pharmaceutically acceptable salts, tautomers, prodrugs, solvates, metabolites, polymorphs, analogs or derivatives thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound or a pharmaceutically acceptable salt, solvate, prodrug, stereoisomer, or tautomer thereof is administered orally. One skilled in the art will recognize the advantages of certain routes of administration.

The dosage regimen utilizing the compound is selected in accordance with a variety of factors including type, species, age, weight, sex and medical condition of the patient; the severity of the condition to be treated; the route of administration; the renal and hepatic function of the patient; and the particular compound or pharmaceutically acceptable salt, solvate, prodrug, stereoisomer, or tautomer thereof employed. An ordinarily skilled physician or veterinarian can readily determine and prescribe the effective amount of the drug required to prevent, counter or arrest the progress of the condition.

Techniques for formulation and administration of the disclosed compound of the application can be found in Remington: the Science and Practice of Pharmacy, 19th edition, Mack Publishing Co., Easton, PA (1995). In an embodiment, the compound described herein, and the pharmaceutically acceptable salts, solvates, prodrugs, stereoisomers, or tautomers thereof, are used in pharmaceutical preparations in combination with a pharmaceutically acceptable carrier or diluent. Suitable pharmaceutically acceptable carriers include inert solid fillers or diluents and sterile aqueous or organic solutions. The compound or pharmaceutically acceptable salts, solvates, prodrugs, stereoisomers, or tautomers thereof will be present in such pharmaceutical compositions in amounts sufficient to provide the desired dosage amount in the range described herein.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present application the skilled artisan can identify and employ other components and methodology useful for practicing the present application.

### Examples

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### Section I: Synthesis of Intermediates

### Synthesis of 3-(6-bromobenzofuran-3-yl)piperidine-2,6-dione (Int 1-A) and 3-(6-(4,4,5,5-tetramethyl-1,3,2-dioxaborolan-2-yl)benzofuran-3-yl)piperidine-2,6-dione (Int 1-A & Int 1-B)

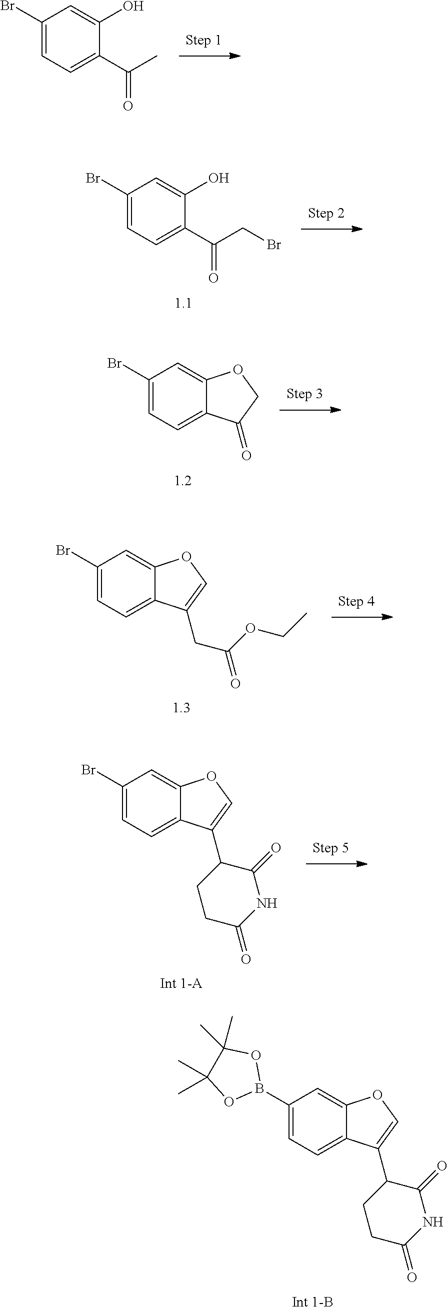

Step 1: To a solution of 1-(4-bromo-2-hydroxy-phenyl) ethanone (10 g, 46.50 mmol, 1 eq) in CHCl₃ (250 mL) and ethyl acetate (250 mL) was added CuBr₂ (25.97 g, 116.26 mmol, 5.44 mL, 2.5 eq) under N₂. The mixture was stirred at 100° C. for 16 hr. The mixture was poured into water and extracted with ethyl acetate. The combined organic layers were washed with brine, dried over Na₂SO₄, filtered and concentrated. The reaction mixture was filtered. The filtrate was concentrated directly to afford crude 2-bromo-1-(4-bromo-2-hydroxy-phenyl) ethenone 1.1 (45 g, 153.09 mmol, 82.30% yield) was obtained as a yellow solid.

Step 2: To a solution of 1.1 (45 g, 153.09 mmol, 1 eq) in DCM (500 mL) was added Et₃N(18.59 g, 183.71 mmol, 25.57 mL, 1.2 eq) at 0° C. under N₂. The mixture was stirred at 20° C. for 2 hr. The mixture was poured into water and extracted with DCM. The combined organic layers were washed with brine, dried over Na₂SO₄, filtered and concentrated. Crude compound 6-bromobenzofuran-3-one 1.2 (44 g, 103.27 mmol, 67.46% yield, 50% purity) was obtained as a red solid.

Step 3: To a solution of 1.2 (44 g, 103.27 mmol, 50% purity, 1 eq) in toluene (500 mL) was added ethyl 2-(triphenyl-25-phosphanylidene)acetate (43.17 g, 123.93 mmol, 1.2 eq) under N₂. The mixture was stirred at 120° C. for 16 hr. The reaction mixture was concentrated under reduced pressure to give a residue. The residue was purified by column chromatography to afford ethyl 2-(6-bromobenzofuran-3-yl)acetate 1.3 (6 g, 21.19 mmol, 20.52% yield) was obtained as a white solid.

Step 4: To a solution of 1.3 (5 g, 12.36 mmol, 70% purity, 1 eq) in DMF (10 mL) was added t-BuOK (1.53 g, 13.60 mmol, 1.1 eq), prop-2-enamide (1.76 g, 24.72 mmol, 1.71 mL, 2 eq) at 0° C. under N₂. The mixture was stirred at 0° C. for 2 hr. The mixture was poured into water and extracted with EtOAc. The combined organic layers were washed with brine, dried over, filtered and concentrated to give a residue. The residue was purified by column chromatography to afford Int 1-A (3.3 g, 10.71 mmol, 86.63% yield) was obtained as a yellow solid. ¹H NMR (400 MHZ, d₆-DMSO) δ 10.91 (s, 1H), 7.94 (s, 1H), 7.89 (d, J=1.2 Hz, 1H), 7.57 (d, J=8.0 Hz, 1H), 7.42 (dd, J=2.0, 8.0 Hz, 1H), 4.15 (dd, J=4.8, 12.0 Hz, 1H), 2.79-2.72 (m, 1H), 2.63-2.54 (m, 1H), 2.37-2.25 (m, 1H), 2.11 (m, 1H). MS: (ESI) m/z calculated for C₁₃H₁₀BrNO₃ [M+H]⁺307.9, found 307.9.

Step 5: A solution of Int 1-A (500 mg, 1.62 mmol, 1 eq), 4,4,5,5-tetramethyl-2-(4,4,5,5-tetramethyl-1,3,2-dioxaborolan-2-yl)-1,3,2-dioxaborolane (494.48 mg, 1.95 mmol, 1.2 eq), KOAc (477.77 mg, 4.87 mmol, 3 eq) in dioxane (10 mL) and H₂O (2 mL) was purged with N₂ for 3 times and then Pd(dppf)Cl₂ (66.26 mg, 81.14 μmol, 0.05 eq) was added in. The mixture was stirred at 100° C. for 16 hr under N₂. The mixture was poured into water and extracted with EtOAc. The combined organic layers were washed with brine, dried over, filtered and concentrated. The residue was purified by column chromatography to afford 3-[6-(4,4,5,5-tetramcthyl-1,3,2-dioxaborolan-2-yl)benzofuran-3-yl]piperidine-2,6-dione Int 1-B (420 mg, 1.18 mmol, 72.87% yield). LCMS: calc. for C₁₉H₂₂BNO₅: 355.2, found: no desired MS signal. ¹H NMR (400 MHZ, DMSO-d₆) § 10.91 (s, 1H), 7.98 (s, 1H), 7.78 (s, 1H), 7.60 (d, J=8.0 Hz, 1H), 7.54 (d, J=7.6 Hz, 1H), 4.12-4.21 (m, 1H), 2.69-2.82 (m, 1H), 2.55-2.61 (m, 1H), 2.27-2.36 (m, 1H), 2.06-2.20 (m, 1H), 1.31 (s, 12H).

### Synthesis of 3-(6-bromo-5-fluorobenzofuran-3-yl)piperidine-2,6-dione (Int 2-A)

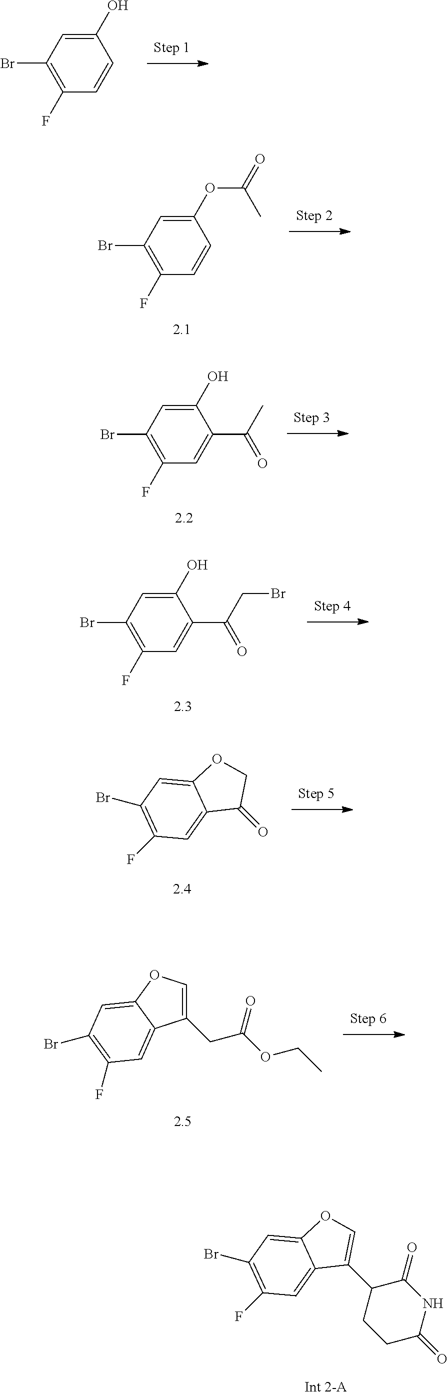

Step 1: To a solution of 3-bromo-4-fluoro-phenol (50 g, 261.78 mmol, 1 eq) in DCM (500 mL) was added in acetyl acetate (32.07 g, 314.14 mmol, 29.42 mL, 1.2 eq) and pyidine (51.77 g, 654.46 mmol, 52.82 mL, 2.5 eq). The mixture was stirred at 20° C. for 16 hr. The reaction mixture was adjusted to pH=3 by aq. AcOH (1M). The resulting mixture were extracted with DCM. The combined organic phase was washed with brine, dried over Na₂SO₄, filtered and concentrated under reduced pressure to give 2.1 (61 g, 261.76 mmol, 99.99% yield) as a white solid. ¹H NMR (400 MHZ, CDCl₃) δ 7.34 (dd, J=2.8, 5.6 Hz, 1H), 7.17-7.11 (m, 1H), 7.04 (m, 1H), 2.30 (s, 3H).

Step 2: To a solution of (3-bromo-4-fluoro-phenyl)acetate (57 g, 244.60 mmol, 1 eq) was added in TfOH (183.54 g, 1.22 mol, 107.97 mL, 5 eq) at 0° C. The mixture was stirred at 60° C. for 3.5 hr. The mixture was poured into cold water and extracted with CH₂C₁₂. The combined organic phase was washed with water, dried over anhydrous Na₂SO₄, filtered and concentrated. 1-(4-Bromo-5-fluoro-2-hydroxy-phenyl) ethanone (2.2, 50 g, 214.56 mmol, 87.72% yield) was obtained as a yellow solid. ¹H NMR (400 MHZ, d₆-DMSO) δ 11.64 (br d, J=1.6 Hz, 1H), 7.79 (d, J=9.2 Hz, 1H), 7.33 (d, J=6.0 Hz, 1H), 2.61 (s, 3H).

Step 3: To a solution of 1-(4-bromo-5-fluoro-2-hydroxy-phenyl) ethanone (43 g, 184.52 mmol, 1 eq) in CHCl₃ (250 mL) and EtOAc (250 mL) was added in CuBr₂ (103.03 g, 461.31 mmol, 21.60 mL, 2.5 eq) under N₂. The mixture was stirred at 100° C. for 16 hr. The mixture was filtered and the filtrate was concentrated. 2-Bromo-1-(4-bromo-5-fluoro-2-hydroxy-phenyl) ethanone (2.3, 65 g, crude) was obtained as a red solid. ¹H NMR (400 MHZ, d₆-DMSO) δ 11.33 (s, 1H), 7.67 (d, J=9.2 Hz, 1H), 7.31 (d, J=5.6 Hz, 1H), 4.85 (s, 2H).

Step 4: To a solution of 2-bromo-1-(4-bromo-5-fluoro-2-hydroxy-phenyl) ethanone (25 g, 80.15 mmol, 1 eq) in DCM (250 mL) was added in TEA (9.73 g, 96.18 mmol, 13.39 mL, 1.2 eq) at 0° C. under N₂ atmosphere. The mixture was stirred at 20° C. for 2 hr, and poured into water and was extracted with DCM. The combined organic layers were washed with brine, dried over Na₂SO₄, filtered and concentrated to give 6-bromo-5-fluoro-benzofuran-3-one (2.4, 18.5 g, 80.08 mmol, 99.92% yield) as red solid. ¹H NMR (400 MHz, d₆-DMSO) δ 7.83 (d, J=4.8 Hz, 1H), 7.67 (d, J=7.2 Hz, 1H), 4.87 (s, 2H).

Step 5: To a solution of 6-bromo-5-fluoro-benzofuran-3-one (6 g, 15.58 mmol, 60% purity, 1 eq) in toluene (40 mL) was added in ethyl 2-(triphenyl-25-phosphanylidene)acetate (6.51 g, 18.70 mmol, 1.2 eq) under N₂. The mixture was stirred at 130° C. for 16 hr. The reaction mixture was concentrated in vacuo directly. The residue was purified by flash silica gel chromatography. Ethyl 2-(6-bromo-5-fluoro-benzofuran-3-yl)acetate (2.5, 2 g, 6.64 mmol, 42.62% yield) was obtained as a yellow solid. ¹H NMR (400 MHZ, d₆-DMSO) δ 8.04-8.00 (m, 2H), 7.62 (d, J=8.8 Hz, 1H), 4.11 (q, J=7.2 Hz, 2H), 3.79 (s, 2H), 1.19 (t, J=7.2 Hz, 3H)

Step 6: To a solution of ethyl 2-(6-bromo-5-fluoro-benzofuran-3-yl)acetate (5 g, 16.61 mmol, 1 eq) in DMF (50 mL) was added in t-BuOK (2.05 g, 18.27 mmol, 1.1 eq) at 0° C. over 0.5 hr under N₂. Then prop-2-enamide (2.36 g, 33.21 mmol, 2.29 mL, 2 eq) was added in dropwise at 0° C. The mixture was stirred at 0° C. for 1.5 hr. The mixture was poured into water and was extracted with EtOAc. The combined organic layers were washed with brine, dried over Na₂SO₄, filtered and concentrated. The crude product was triturated with PE:EA=10:1 at 20° C. for 1 hr to afford Int 2-A (2.8 g, 8.59 mmol, 51.71% yield) as a yellow solid. ¹H NMR (400 MHZ, d₆-DMSO) δ 10.92 (s, 1H), 8.06 (d, J=5.6 Hz, 1H), 8.03 (s, 1H), 7.68 (d, J=8.8 Hz, 1H), 4.13 (dd, J=4.8, 12.4 Hz, 1H), 2.77-2.67 (m, 1H), 2.57-2.53 (m, 1H), 2.42-2.30 (m, 1H), 2.13-2.05 (m, 1H).

### Synthesis of 3-[5-fluoro-6-(4,4,5,5-tetramethyl-1,3,2-dioxaborolan-2-yl)benzofuran-3-yl]piperidine-2,6-dione (Int 2-B)

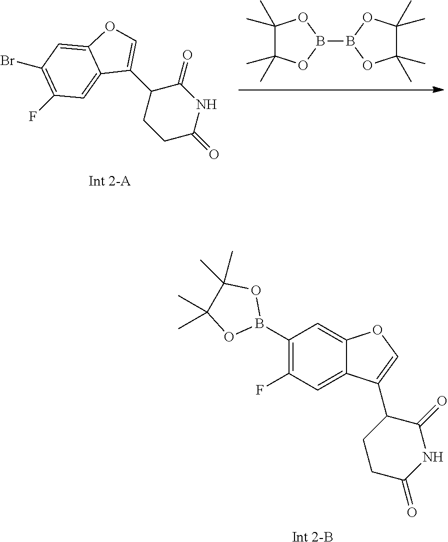

To a mixture of 4,4,5,5-tetramethyl-2-(4,4,5,5-tetramethyl-1,3,2-dioxaborolan-2-yl)-1,3,2-dioxaborolane (1.99 g, 7.82 mmol, 1.5 eq), Int 2-A (1.7 g, 5.21 mmol, 1 eq), KOAc (1.53 g, 15.64 mmol, 3 eq), Pd(dppf)Cl₂ (212.85 mg, 260.64 μmol, 0.05 eq) in dioxane (5 mL) and H₂O (0.5 mL) was degassed and purged with N₂ for 3 times, and then the mixture was stirred at 100° C. for 16 hr under N₂ atmosphere. LCMS showed desired compound was detected. The reaction mixture was concentrated directly. The crude product was purified by reversed-phase HPLC. Int 2-B (0.55 g, 1.47 mmol, 28.27% yield) was obtained as a white solid. ¹H NMR (400 MHZ, d₆-DMSO) $10.89 (s, 1H), 8.04 (s, 1H), 7.72 (d, J=4.4 Hz, 1H), 7.41 (d, J=9.2 Hz, 1H), 4.13 (dd, J=4.8, 12.4 Hz, 1H), 2.78-2.66 (m, 1H), 2.57-2.52 (m, 1H), 2.40-2.28 (m, 1H), 2.15-2.04 (m, 1H), 1.31 (s, 12H). MS: (ESI) m/z calculated for C₁₉H₂₁BFNO₅ [M+H]⁺374.1, found 374.1.

### Synthesis of 3-(6-bromobenzo[d]isoxazol-3-yl)piperidine-2,6-dione (Int 3-A)

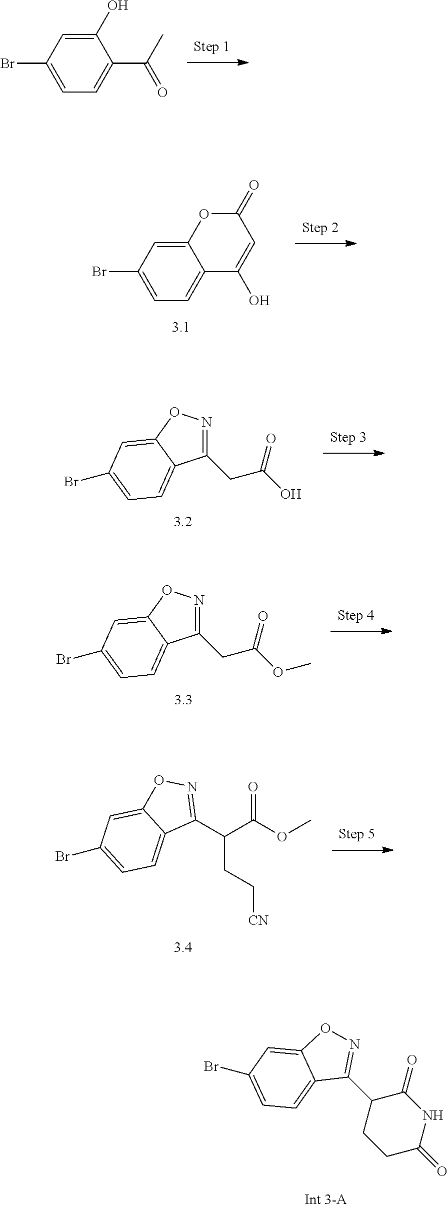 [SMILES: CC(=O)c1ccc(Br)cc1O ; O=c1cc(O)c2ccc(Br)cc2o1 ; O=C(O)Cc1noc2cc(Br)ccc12 ; COC(=O)Cc1noc2cc(Br)ccc12 ; COC(=O)C(CCC#N)c1noc2cc(Br)ccc12 ; O=C1CCC(c2noc3cc(Br)ccc23)C(=O)N1]

Step 1: To a stirred suspension of NaH (14 g, 350.00 mmol, 60% purity in mineral oil) in toluene (400 mL) was added 1-(4-bromo-2-hydroxy-phenyl) ethanone (25 g, 116.26 mmol) in toluene (50 mL) below 20° C. After stirred at 20° C. for 30 min, diethyl carbonate (27.30 g, 231.10 mmol, 28.00 mL) in toluene (50 mL) was added to above mixture slowly at 20° C. The resulting mixture was stirred at 120° C. for 20 h under N₂. A yellow suspension was formed. The reaction mixture was quenched with water below 10° C. After adjusted pH=5 with 1M HCl aqueous solution, the resulting mixture was stirred at 20° C. for 30 min, and was extracted with EtOAc. The combined organic phase was washed with brine, dried over anhydrous Na₂SO₄ and concentrated. The residue was triturated with EtOAc to give 7-bromo-4-hydroxy-chromen-2-one (3.1, 22 g, 91.27 mmol, yield: 78.5%) as a white solid. ¹H NMR (400 MHZ, DMSO-d₆) δ 12.72 (s, 1H), 7.72 (d, J=8.4 Hz, 1H), 7.68 (s, 1H), 7.53 (d, J=8.4 Hz, 1H), 5.60 (s, 1H).

Step 2: To a stirred solution of hydroxylamine hydrochloride (37.9 g, 545.40 mmol) in EtOH (500 mL) was added sodium ethanolate (40 g, 558.41 mmol, 95% purity) below 10° C. 7-Bromo-4-hydroxy-chromen-2-one (22 g, 91.27 mmol) was added to the mixture below 10° C. The reaction mixture was stirred at 80° C. for 60 h under N₂, and a white suspension was formed. The reaction mixture was cooled to 40° C. and then it was adjusted to pH=2 with 1 M HCl aqueous solution. After stirred at 20° C. for 1 h, the mixture was concentrated under reduced pressure. The residue was purified by Combi Flash to give 2-(6-bromo-1,2-benzoxazol-3-yl) acetic acid (3.2, 20.5 g, 80.06 mmol, yield: 87.7%) as a white solid. ¹H NMR (400 MHZ, DMSO-d₆) δ 12.77 (brs, 1H), 8.10 (s, 1H), 7.81 (d, J=8.4 Hz, 1H), 7.58 (d, J=8.4 Hz, 1H), 4.11 (s, 2H).

Step 3: To a stirred solution of con. H₂SO₄ (14.72 g, 150.08 mmol, 8 mL) in MeOH (280 mL) was added in a mixture of 2-(6-bromo-1,2-benzoxazol-3-yl) acetic acid (20.5 g, 80.06 mmol) in MeOH (40 mL). The resulting mixture was stirred at 70° C. for 6 h. The resulting brown reaction mixture was neutralized with NaHCO₃ solid (3 g) and then it was concentrated under reduced pressure. The residue was purified Combi Flash to give methyl 2-(6-bromo-1,2-benzoxazol-3-yl)acetate (3.3, 4.2 g, 52.58 mmol, yield: 65.7%) as a white solid. ¹H NMR (400 MHZ, DMSO-d₆) δ 8.13 (s, 1H), 7.82 (d, J=8.4 Hz, 1H), 7.60 (d, J=8.4 Hz, 1H), 4.24 (s, 2H), 3.67 (s, 3H).

Step 4: To a stirred solution of methyl 2-(6-bromo-1,2-benzoxazol-3-yl)acetate (14.2 g, 52.58 mmol) in THF (150 mL) was added in NaH (2.12 g, 53.01 mmol, 60% purity in mineral oil) at 0° C. After stirred at 0° C. for 30 min, 3-bromopropanenitrile (7.04 g, 52.58 mmol, 4.32 mL) in THF (30 mL) was added to the mixture dropwise at 0° C. and then it was stirred at 0° C. for 1 h. To the resulting brown reaction mixture was added sat. aq. NH₄Cl (150 mL) below 10° C. The mixture was diluted with EtOAc and washed with water. The organic phase was dried over anhydrous Na₂SO₄ and concentrated in vacuo to give a white residue. The residue was purified by Combi Flash to give methyl 2-(6-bromo-1,2-benzoxazol-3-yl)-4-cyano-butanoate (3.4, 7.1 g, 21.97 mmol, yield: 41.8%) as a white solid. ¹H NMR (400 MHZ, DMSO-d₆) δ 8.16 (s, 1H), 7.83 (d, J=8.4 Hz, 1H), 7.61 (d, J=8.4 Hz, 1H), 4.44-4.49 (m, 1H), 3.66 (s, 3H), 2.58-2.62 (s, 2H), 2.45-2.49 (m, 1H), 2.30-2.38 (m, 1H).

Step 5: A solution of methyl 2-(6-bromo-1,2-benzoxazol-3-yl)-4-cyano-butanoate (1.7 g, 5.26 mmol) in H₂SO₄ (5.52 g, 56.28 mmol, 3 mL) and HOAc (21 mL) was stirred at 105° C. for 2.5 h under N₂. The reaction mixture was diluted with water and then was adjusted to pH=3 with sat. aq. NaHCO₃. The resulting mixture was extracted with DCM. The combined organic phase was washed with brine, dried over anhydrous Na₂SO₄ and concentrated. The residue was purified by Combi Flash to give 3-(6-bromo-1,2-benzoxazol-3-yl)piperidine-2,6-dione (Int 3-A, 3.02 g, 9.77 mmol, yield: 46.4%) as an off-white solid. ¹H NMR (400 MHZ, DMSO-d₆) δ 11.11 (s, 1H), 8.14 (s, 1H), 7.83 (d, J=8.4 Hz, 1H), 7.58 (d, J=8.0 Hz, 1H), 4.60-4.65 (m, 1H), 2.72-2.80 (m, 1H), 2.52-2.64 (m, 2H), 2.18-2.22 (m, 1H).

### Synthesis of 3-(6-(4,4,5,5-tetramethyl-1,3,2-dioxaborolan-2-yl)benzo[d]isoxazol-3-yl)piperidine-2,6-dione (Int 3-B)

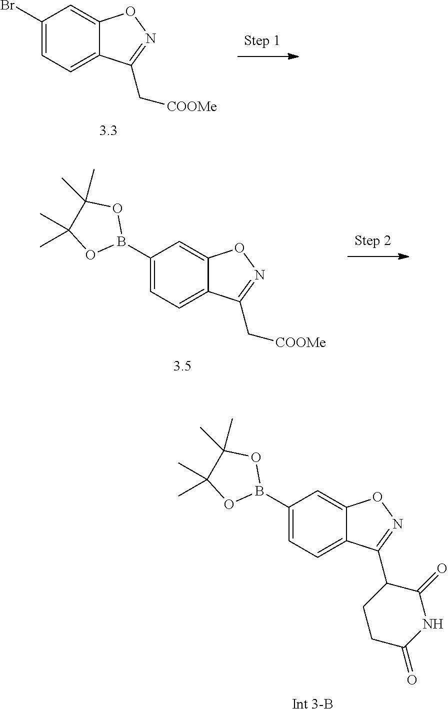

Step 1: To a solution of methyl 2-(6-bromo-1,2-benzoxazol-3-yl)acetate 3.3 (20.0 g, 74.05 mmol, 1 eq), AcOK (29.07 g, 296.21 mmol, 4 eq) and Pin₂B₂ (22.57 g, 88.86 mmol, 1.2 eq) in dioxane (200 mL) was added in Pd(PPh3)₂Cl₂ (5.20 g, 7.41 mmol, 0.1 eq). The mixture was stirred at 100° C. for 6 h under N₂. The reaction mixture was poured into water and extracted with EtOAc. The combined organic layer was washed with brine, dried over anhydrous Na₂SO₄ and concentrated. The residue was purified by flash chromatography to afford methyl 2-[6-(4,4,5,5-tetramethyl-1,3,2-dioxaborolan-2-yl)-1,2-benzoxazol-3-yl]acetate (3.5, 22.5 g, 70.95 mmol, 95.80% yield) as yellow oil. ¹H NMR (400 MHZ, DMSO-d₆) δ 7.93 (s, 1H), 7.87 (d, J=8.0 Hz, 1H), 7.67 (d, J=7.6 Hz, 1H), 4.25 (s, 2H), 3.67 (s, 3H), 1.33 (s, 12H).

Step 2: To a mixture of 3.5 (22.0 g, 69.37 mmol, 1 eq) and t-BuOK (1 M in THF, 70.02 mmol, 70.02 mL, 1.01 eq) in THF (130 mL) was added in prop-2-enamide (4.93 g, 69.37 mmol, 4.79 mL, 1 eq) at 0° C. The mixture was stirred at 0° C. for 1 h. Then was added in HCl/dioxane (4 M, 50 mL). The yellow residue was purified by flash chromatography to afford 3-[6-(4,4,5,5-tetramethyl-1,3,2-dioxaborolan-2-yl)-1,2-benzoxazol-3-yl]piperidine-2,6-dione (Int 3-B, 7.5 g, 21.06 mmol, 30.35% yield) as white solid. ¹H NMR (400 MHZ, DMSO-d₆) δ 11.12 (brs, 1H), 7.94 (s, 1H), 7.89 (d, J=8.0 Hz, 1H), 7.65 (d, J=8.4 Hz, 1H), 4.60-4.68 (m, 1H), 2.70-2.80 (m, 1H), 2.52-2.62 (m, 2H), 2.15-2.25 (m, 1H), 1.33 (s, 12H).

### Synthesis of 3-(6-bromo-5-fluorobenzo[d]isoxazol-3-yl)piperidine-2,6-dione (Int 4-A)

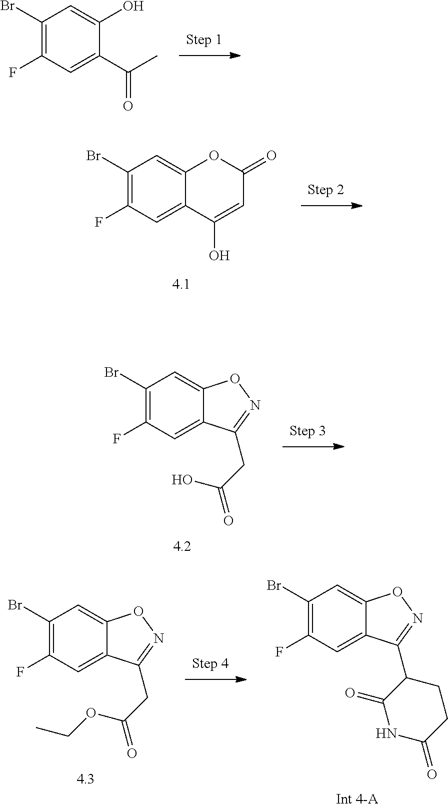

Step 1: To a solution of 1-(4-bromo-5-fluoro-2-hydroxy-phenyl) ethanone (20 g, 85.82 mmol, 1 eq) in toluene (300 mL) was added in portions NaH (10.30 g, 257.47 mmol, 60% purity, 3 eq) at 0° C. The mixture was stirred at this temperature for 30 min, then diethyl carbonate (20.28 g, 171.65 mmol, 20.80 mL, 2 eq) in toluene (300 mL) was added in dropwise at 20° C. The resulting mixture was stirred at 120° C. for 16 hr, and cooled to below 10° C. and quenched with water. After adjusted pH=5 with 1M HCl aqueous solution, the reaction mixture was filtered and the filter cake was dried in vacuo to afford 7-bromo-6-fluoro-4-hydroxy-chromen-2-one (4.1, 20 g, 77.21 mmol, 89.96% yield) as a yellow solid. ¹H NMR (400 MHZ, d₆-DMSO) δ 7.89 (d, J=5.6 Hz., 1H), 7.68 (d, J=8.4 Hz., 1H), 5.66 (s, 1H).

Step 2: To a solution of hydroxylamine hydrochloride (14.73 g, 138.98 mmol, 3 eq, HCl) in EtOH (100 mL) was added EtONa (9.46 g, 138.98 mmol, 3 eq) at 0° C. Then 7-bromo-6-fluoro-4-hydroxy-chromen-2-one (12 g, 46.33 mmol, 1 eq) was added dropwise at 0° C. The resulting mixture was stirred at 80° C. for 16 hr. The reaction was adjusted to make pH=2 using 1M HCl aqueous solution, then extracted with EtOAc. The combined organic phase was washed with brine, dried over anhydrous Na₂SO₄ and concentrated in vacuo to afford 2-(6-bromo-5-fluoro-1,2-benzoxazol-3-yl) acetic acid (4.2, 12.9 g, crude) as a white solid. ¹H NMR (400 MHZ, d₆-DMSO) δ 12.95 (br d, J=12.0 Hz, 1H), 8.31 (d, J=5.2 Hz, 1H), 7.93 (d, J=8.0 Hz, 1H), 4.11 (s, 2H).

Step 3: To a solution of H₂SO₄ (9.23 g, 94.15 mmol, 5.02 mL, 2 eq) in EtOH (50 mL) was added in 2-(6-bromo-5-fluoro-1,2-benzoxazol-3-yl) acetic acid (4.2, 12.9 g, 47.07 mmol, 1 eq) in EtOH (70 mL). The mixture was stirred at 70° C. for 7 hr then was quenched with aq. NaHCO₃dropwise. The mixture was extracted with EtOAc. The combined organic phase was washed with brine, dried over anhydrous Na₂SO₄, filtered and concentrated. The residue was purified by flash silica gel chromatography to afford ethyl 2-(6-bromo-5-fluoro-1,2-benzoxazol-3-yl)acetate (4.3, 8.2 g, 27.14 mmol, 57.66% yield) as a white solid. ¹H NMR (400 MHZ, d₆-DMSO) δ 8.31 (d, J=5.2 Hz, 1H), 7.93 (d, J=8.0 Hz, 1H), 4.21 (s, 2H), 4.14 (q, J=7.2 Hz, 2H), 1.19 (t, J=7.2 Hz, 3H).

Step 4: To a solution of ethyl 2-(6-bromo-5-fluoro-1,2-benzoxazol-3-yl)acetate (4.3, 5.5 g, 18.21 mmol, 1 eq) in DMF (40 mL) was added in dropwisely t-BuOK (2.25 g, 20.03 mmol, 1.1 eq) at 0° C. After addition, the mixture was stirred at this temperature for 30 min, and then prop-2-enamide (2.59 g, 36.41 mmol, 2.51 mL, 2 eq) in DMF (10 mL) was added in dropwise at 0° C. under N₂. The mixture was stirred at 0° C. for 1 hr and poured into water and was extracted by EtOAc. The combined organic layer was washed with brine, dried over Na₂SO₄, filtered and concentrated. The crude product was triturated with DCM at 20° C. for 1 hr to afford 3-(6-bromo-5-fluoro-1,2-benzoxazol-3-yl)piperidine-2,6-dione (Int 4-A, 2 g, 6.11 mmol, 33.58% yield) as a yellow solid. ¹H NMR (400 MHZ, d₆-DMSO) δ 11.12 (br s, 1H), 8.34 (d, J=5.6 Hz, 1H), 8.00 (d, J=8.0 Hz, 1H), 4.60 (dd, J=4.8, 12.0 Hz, 1H), 2.82-2.70 (m, 1H), 2.65-2.55 (m, 2H), 2.19 (m, 1H)

### Synthesis of 3-(6-bromo-5-methyl-benzofuran-3-yl)piperidine-2,6-dione (Int 5)

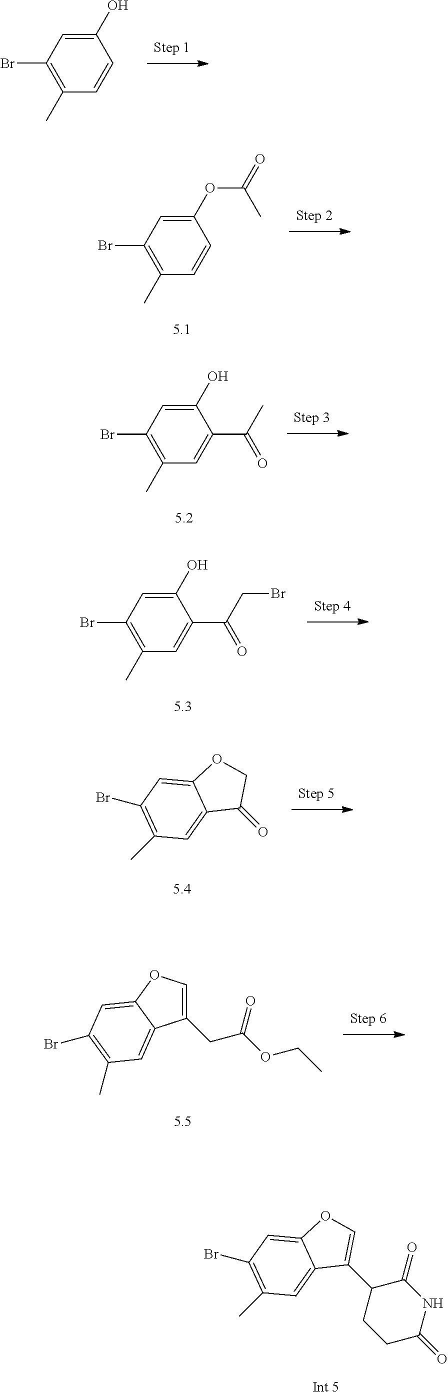

Step 1: To a solution of 3-bromo-4-methyl-phenol (20 g, 106.93 mmol, 1 eq) and Ac₂O (13.10 g, 128.32 mmol, 1.2 eq) in DCM (200 mL) was added dropwise pyridine (21.15 g, 267.33 mmol, 2.5 eq) at 0° C. After stirred at 25° C. for 16 h, the reaction mixture was concentrated. Water (300 mL) was added to the residue. The resulting mixture was extracted with EtOAc (200 mL×2). The combined organic phase was washed with aq. CuSO₄ (500 mL), dried over anhydrous Na₂SO₄, filtered and concentrated under reduced pressure to give (3-bromo-4-methyl-phenyl)acetate 5.1 (23 g, 84.51% yield) as a yellow oil. ¹H NMR (400 MHZ, CDCl₃) δ 7.31 (d, J=2.4 Hz, 1H), 7.24 (d, J=8.4 Hz, 1H), 6.96 (dd, J=8.4, 2.4 Hz, 1H), 2.39 (s, 3H), 2.29 (s, 3H).

Step 2: To a solution of 5.1 (23 g, 100.41 mmol, 1 eq) was added dropwise TfOH (50 mL) at 0° C. The mixture was stirred at 60° C. for 3 h. The reaction mixture was poured into ice water (500 mL) slowly. The mixture was filtered and the cake was concentrated under reduced pressure to give 1-(4-bromo-2-hydroxy-5-methyl-phenyl) ethanone 5.2 (22.5 g, 92.93% yield) as an off-white solid. ¹H NMR (400 MHZ, CDCl₃) δ 12.07 (s, 1H), 7.55 (s, 1H), 7.22 (s, 1H), 2.61 (s, 3H), 2.36 (s, 3H).

Step 3: A mixture of 5.2 (22 g, 96.04 mmol, 1 eq), CuBr₂ (32.18 g, 144.06 mmol, 1.5 eq) in CHCl₃ (200 mL) and EtOAc (200 mL) was degassed and purged with N₂ for 3 times, and then the mixture was stirred at 100° C. for 16 h under N₂ atmosphere. The mixture was filtered, and the filtrate was concentrated under reduced pressure to give 2-bromo-1-(4-bromo-2-hydroxy-5-methyl-phenyl) ethanone 5.3 (31 g, 78.61% yield) as a dark brown solid.

¹H NMR (400 MHZ, CDCl₃) δ 11.60 (s, 1H), 7.61-7.63 (m, 1H), 7.33 (s, 1H), 4.46 (s, 2H), 2.42-2.45 (m, 3H).

Step 4: A mixture of 5.3 (15 g, 36.53 mmol, 75% purity, 1 eq) in DCM (150 mL) was degassed and purged with N₂ for 3 times, and then was added TEA (4.44 g, 43.84 mmol, 1.2 eq) dropwise at 0° C. The mixture was stirred at 25° C. for 2 h under N₂ atmosphere aq.NH₄Cl (500 mL) was added to the mixture. The resulting mixture was extracted with DCM (300 mL×2). The combined organic phase was washed with water (500 mL), dried over anhydrous Na₂SO₄, filtered and concentrated under reduced pressure to give residue. The residue was purified by flash silica gel chromatography to give 6-bromo-5-methyl-benzofuran-3-one 5.4 (12.4 g, 67.28% yield) as a yellow solid. ¹H NMR (400 MHZ, CDCl₃) δ 7.50 (s, 1H), 7.40 (s, 1H), 4.60 (s, 2H), 2.40 (s, 3H).

Step 5: A mixture of 5.4 (6 g, 26.43 mmol, 1 eq), ethyl 2-(triphenyl-25-phosphanylidene)acetate (11.05 g, 31.71 mmol, 1.2 eq) in Tol. (120 mL) was degassed and purged with N₂ for 3 times, and then the mixture was stirred at 130° C. for 16 h under N₂ atmosphere. The reaction mixture was concentrated under reduced pressure to give residue. The residue was purified by flash silica gel chromatography to give ethyl 2-(6-bromo-5-methyl-benzofuran-3-yl)acetate 5.5 (6.1 g, 73.8% yield) as a yellow solid. 1HNMR (400 MHZ, CDCl₃) δ 7.70 (s, 1H), 7.58 (s, 1H), 7.43 (s, 1H), 4.20 (q, J=8.0 Hz, 2H), 3.66 (s, 2H), 2.49 (s, 3H), 1.28 (t, J=8.0 Hz, 3H).

Step 6: A mixture of ethyl 5.5 (6.1 g, 20.53 mmol, 1 eq), prop-2-enamide (4.38 g, 61.59 mmol, 3 eq), t-BuOK (2.99 g, 26.69 mmol, 1.3 eq) in DMF (50 mL) was degassed and purged with N₂ for 3 times, and then the mixture was stirred at 0° C. for 1.5 h under N₂ atmosphere. Water (200 mL) was added to the mixture. The resulting mixture was extracted with EtOAc (150 mL×2). The combined organic phase was washed with brine (300 mL×2), dried over anhydrous Na₂SO₄, filtered and concentrated under reduced pressure to give a residue. The crude product was triturated with EtOAc (10 mL) at 20° C. for 1 h. The mixture was filtered and the cake was concentrated under reduced pressure to give Int 5 (4.1 g, 55.80% yield) as a light yellow solid. 1HNMR (400 MHZ, DMSO-d₆) § 10.90 (s, 1H), 7.89 (d, J=4.0 Hz, 2H), 7.60 (s, 1H), 4.11 (dd, J=12.0, 4.8 Hz, 1H), 2.68-2.80 (m, 1H), 2.53-2.63 (m, 1H), 2.38-2.43 (s, 3H), 2.34 (m, 1H), 2.04-2.14 (m, 1H).

### Synthesis of 3-(6-bromo-7-fluoro-benzofuran-3-yl)piperidine-2,6-dione (Int 6)

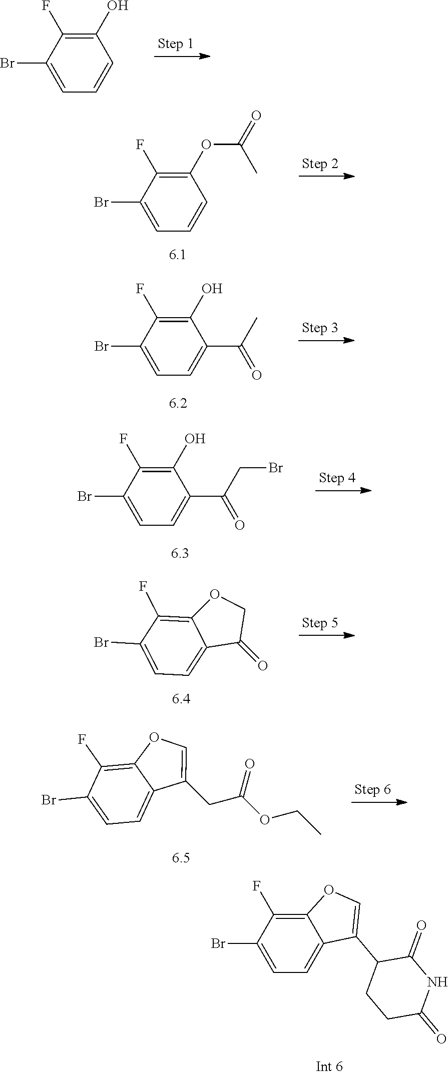

Step 1: To a solution of 3-bromo-2-fluoro-phenol (20.0 g, 104.71 mmol, 1 eq) and Ac₂O (12.83 g, 125.66 mmol, 1.2 eq) in CH₂Cl₂ (150 mL) was added pyridine (20.71 g, 261.78 mmol, 2.5 eq). The mixture was stirred at 25° C. for 16 h. The reaction mixture was concentrated. DCM (200 mL) was added to the residue, then was washed with aq. CuSO₄ (200 mL), dried over anhydrous Na₂SO₄, filtered and concentrated to give (3-bromo-2-fluoro-phenyl)acetate 6.1 (24 g, crude) as a colorless oil. ¹HNMR (400 MHZ, CDCl₃) δ 7.47-7.41 (m, 1H), 7.13-7.01 (m, 2H), 2.35 (s, 3H).

Step 2: A mixture of 6.1 (24 g, 102.99 mmol, 1 eq) was treated trifluoromethanesulfonic acid (48.07 g, 320.30 mmol, 3.11 eq) slowly at 0° C. After addition, the mixture was stirred at 60° C. for 3.5 h. The mixture was poured into cold water (200 mL) and extracted with CH₂Cl₂ (100 mL×3). The combined organic phase was washed with water (100 mL×3), dried over anhydrous Na₂SO₄, filtered and concentrated to give 1-(4-bromo-3-fluoro-2-hydroxy-phenyl) ethanone 6.2 (24.0 g, crude) as a dark green solid. ¹HNMR (400 MHZ, CDCl₃) δ 12.45 (s, 1H), 7.40 (dd, J=4.0, 8.8 Hz, 1H), 7.08 (dd, J=8.0, 8.8 Hz, 1H), 2.64 (s, 3H).

Step 3: To a solution of 6.2 (24 g, 102.99 mmol, 1 eq) in CHCl₃ (100 mL) and EtOAc (100 mL) was added CuBr₂ (27.60 g, 123.59 mmol, 1.2 eq) under N₂. The mixture was stirred at 100° C. for 16 hr. The mixture was filtered, and the filtrate was concentrated to give 2-bromo-1-(4-bromo-3-fluoro-2-hydroxy-phenyl) ethanone 6.3 (25.0 g, crude) as a black oil. HNMR (400 MHZ, CDCl₃) δ 12.45 (s, 1H), 7.46-7.39 (m, 2H), 7.16-7.06 (m, 2H), 4.40 (s, 1H), 2.65 (s, 3H).

Step 4: To a solution of 6.3 (22.0 g, 49.37 mmol, 70% purity, 1 eq) in DCM (100 mL) was added TEA (5.99 g, 59.24 mmol, 1.2 eq) at 0° C. under N₂ atmosphere. The mixture was stirred at 25° C. for 3 h. The mixture was diluted with H₂O (100 mL), then was extracted with DCM (200 mL×2). The organic layer was washed with aq. NH₄Cl (150 mL), dried over Na₂SO₄, filtered, and concentrated under reduced pressure to give a residue. The residue was purified by flash silica gel chromatography to give 6-bromo-7-fluoro-benzofuran-3-one 6.4 (2.0 g, crude) as a yellow solid. ¹HNMR (400 MHZ, CDCl₃) δ 7.35-7.31 (m, 1H), 7.26-7.23 (m, 1H), 4.78-4.68 (m, 2H).

Step 5: A mixture of 6.4 (2.0 g, 8.66 mmol, 1 eq) and ethyl 2-(triphenyl-25-phosphanylidene)acetate (3.62 g, 10.39 mmol, 1.2 eq) in toluene (20 mL) was degassed and purged with N₂ for 3 times, and then the mixture was stirred at 130° C. for 16 h under N₂ atmosphere. The mixture was concentrated under reduced pressure to give a residue. The residue was purified by flash silica gel chromatography to give ethyl 2-(6-bromo-7-fluoro-benzofuran-3-yl)acetate 6.5 (1.7 g, crude) as a yellow oil. ¹HNMR (400 MHz, DMSO-d₆) δ 8.05 (s, 1H), 7.53 (dd, J=8.0, 8.4 Hz, 1H), 7.42 (d, J=8.4 Hz, 1H), 4.14-4.08 (m, 2H), 3.83 (d, J=0.8 Hz, 2H), 1.19 (t, J=7.2 Hz, 3H).

Step 6: To a solution of ethyl 6.5 (1.7 g, 5.65 mmol, 1 eq) in DMF (15 mL) was added t-BuOK (760.23 mg, 6.77 mmol, 1.2 eq) at 0° C. under N₂. Then prop-2-enamide (1.20 g, 16.94 mmol, 1.17 mL, 3 eq) was added dropwise at 0° C. The mixture was stirred at 0° C. for 1.5 h. The mixture was diluted with H₂O (50 mL) and extracted with EtOAc (50 mL×2). The organic layer was washed with brine (50 mL), dried over Na₂SO₄, filtered and concentrated under reduced pressure to give a residue. The crude product was triturated with PE: EtOAc=10:1 at 20° C. for 1 h and then filtered, the cake was concentrated to give Int 6 (0.9 g, crude) as a yellow solid. ¹HNMR (400 MHZ, DMSO-d₆) δ 10.94 (s, 1H), 8.07 (s, 1H), 7.52 (dd, J=8.0, 8.0 Hz, 1H), 7.42 (d, J=8.4 Hz, 1H), 4.24-4.16 (m, 1H), 2.77-2.69 (m, 1H), 2.59 (td, J=4.0, 12.0 Hz, 1H), 2.35-2.32 (m, 1H), 2.17-2.08 (m, 1H).

### Synthesis of 3-(6-bromo-4-fluoro-benzofuran-3-yl)piperidine-2,6-dione (Int 7)

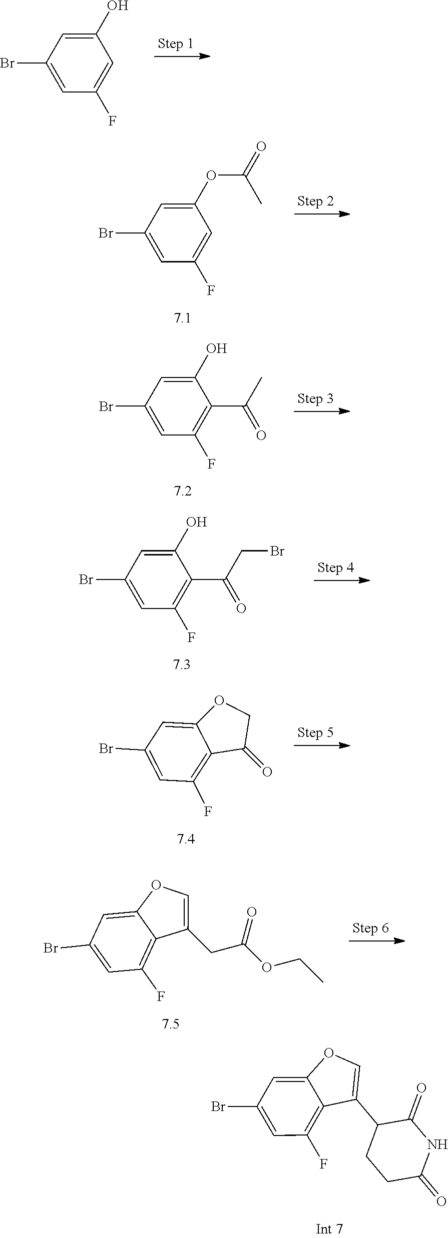

Step 1: To a solution of 3-bromo-5-fluoro-phenol (5 g, 26.18 mmol, 1 eq) in DCM (50 mL) was added pyridine (5.18 g, 65.45 mmol, 5.28 mL, 2.5 eq) and acetyl acetate (4.01 g, 39.27 mmol, 3.68 mL, 1.5 eq). The mixture was stirred at 25° C. for 16 h. The reaction mixture was concentrated. CuSO₄ (50 mL) was added to the residue. The resulting mixture was extracted with EtOAc (100 mL×3). The combined organic phase was washed with brine (100 mL), dried over anhydrous Na₂SO₄, filtered and concentrated under reduced pressure to give (3-bromo-5-fluoro-phenyl)acetate 7.1 (4.63 g, 75.91% yield) as a yellow oil. ¹H NMR (400 MHZ, CDCl₃) δ 7.15 (m, 1H), 7.17-7.13 (m, 1H), 6.85 (m, 1H), 2.30 (s, 3H).

Step 2: A solution of 7.1 (4.3 g, 18.45 mmol, 1 eq) in TfOH (13.85 g, 92.26 mmol, eq) was stirred at 60° C. for 3.5 h. The reaction was quenched with ice water (50 mL) dropwise. The resulting mixture was extracted with EtOAc (50 mL×3). The combined organic phase was washed with brine (50 mL), dried over anhydrous Na₂SO₄, filtered and concentrated under reduced pressure to give 1-(4-bromo-2-fluoro-6-hydroxy-phenyl) ethanone 7.2 (4.2 g, 97.67% yield) as a green solid. ¹H NMR (400 MHZ, DMSO-d₆) δ 11.81 (s, 1H), 7.09 (dd, J=12.0, 2.4 Hz, 1H), 7.00 (t, J=4.0 Hz, 1H), 2.54 (d, J=4.0 Hz, 3H).

Step 3: To a solution of 7.2 (4.20 g, 18.02 mmol, 1 eq) in CHCl₃ (20 mL) and EtOAc (20 mL) was added CuBr₂ (10.06 g, 45.06 mmol, 2.5 eq). The mixture was stirred at 100° C. for 16 h under N₂ atmosphere. The reaction mixture was filtered. The reaction mixture was concentrated directly to give ethyl 2-(6-chlorofuro[3,2-c]pyridin-3-yl)acetate 7.3 (9.6 g, 61.54% yield) as a light-yellow solid. ¹H NMR (400 MHZ, CDCl₃)_{8 12.29} (s, 1H), 7.15-7.02 (m, 1H), 6.91-6.85 (m, 1H), 4.53 (d, J=4.0 Hz, 2H).

Step 4: To a solution of 7.3 (5.90 g, 15.13 mmol, 80% purity, 1 eq) in DCM (50 mL) was added Et₃N(1.84 g, 18.16 mmol, 2.53 mL, 1.2 eq) at 0° C. The mixture was stirred at 25° C. for 2 h. The reaction mixture was concentrated. The residue was purified by flash silica gel chromatography to give 6-bromo-4-fluoro-benzofuran-3-one 7.4 (1.25 g, 35.76% yield) as a red oil. ¹H NMR (400 MHZ, CDCl₃) δ 7.16 (s, 1H), 6.93 (d, J=8.00 Hz, 1H), 4.67 (s, 2H).

Step 5: To a solution of 7.4 (1.25 g, 5.41 mmol, 1 eq) and ethyl 2-(triphenyl-25-phosphanylidene)acetate (2.26 g, 6.49 mmol, 1.2 eq) in Tol. (15 mL). The mixture was stirred at 130° C. for 16 h. The reaction mixture was concentrated directly. The residue was purified by flash silica gel chromatography to give ethyl 2-(6-bromo-4-fluoro-benzofuran-3-yl)acetate 7.5 (800 mg, 49.16% yield) as a yellow oil. ¹H NMR (400 MHZ, CDCl₃) δ 7.57 (s, 1H), 7.48 (d, J=1.20 Hz, 1H), 7.10 (m, 1H), 4.22 (m, 2H), 3.79 (m, 2H), 1.29 (m, 3H).

Step 6: To a solution of 7.5 (3.5 g, 11.62 mmol, 1 eq) in DMF (20 mL) was added t-BuOK (1.70 g, 15.11 mmol, 1.3 eq) at 0° C. and purged with N₂ for 3 times. After addition, the mixture was stirred at this temperature for 0.5 h, and then prop-2-enamide (2.48 g, 34.87 mmol, 2.41 mL, 3 eq) in DMF (10 mL) was added dropwise at 0° C. The resulting mixture was stirred at 0° C. for 1.5 h under N₂ atmosphere. The reaction mixture was concentrated directly. The residue was re-crystallized from (Petroleum ether/Ethyl acetate=10:1)₂₀ mL at 25° C. filtered and concentrated under reduced pressure to give Int 7 (2.6 g, 57.61% yield) as a light yellow solid. ¹H NMR (400 MHZ, DMSO-d₆) δ 10.97-10.88 (m, 1H), 8.03-7.99 (m, 1H), 7.86-7.77 (m, 1H), 7.42-7.34 (m, 1H), 4.21-4.15 (m, 1H), 2.85-2.74 (m, 1H), 2.61-2.56 (m, 1H), 2.14-2.21 (m, 1H), 2.1-2.041 (m, 1H).

### Synthesis of 3-(6-chlorofuro[2,3-b]pyridin-3-yl)piperidine-2,6-dione (Int 8)

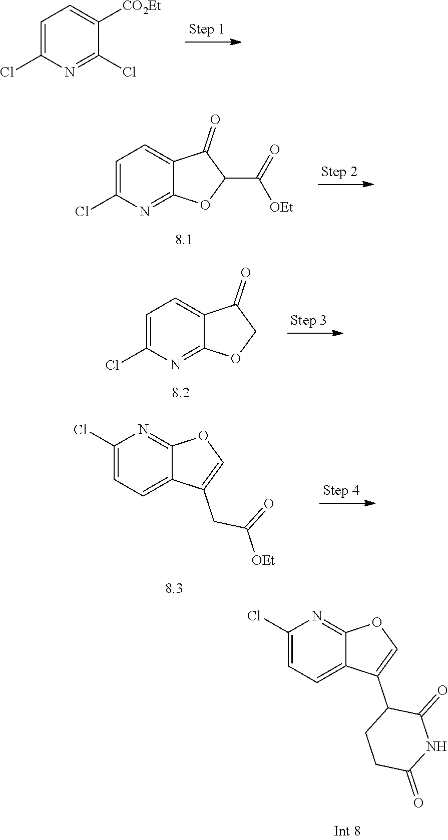 [SMILES: CCOC(=O)c1ccc(Cl)nc1Cl ; CCOC(=O)C1Oc2nc(Cl)ccc2C1=O ; O=C1COc2nc(Cl)ccc21 ; CCOC(=O)Cc1coc2nc(Cl)ccc12 ; O=C1CCC(c2coc3nc(Cl)ccc23)C(=O)N1]

Step 1: To a solution of NaH (4.54 g, 113.61 mmol, 60% purity, 2.5 eq) in DME (90 mL) was added dropwise ethyl 2-hydroxyacetate (11.83 g, 113.61 mmol, 2.5 eq) at 0° C. After addition, the mixture was stirred at 0° C. for 0.5 h, and then ethyl 2,6-dichloropyridine-3-carboxylate (10 g, 45.44 mmol, 1 eq) in DME (10 mL) was added dropwise at 20° C. The resulting mixture was stirred at 75° C. for 2.5 h. The reaction mixture was quenched by H₂O (25 mL) and concentrated. The crude product was triturated with EA: H₂O (25:40 mL) at 20° C. for 20 min. The mixture was filtered and the cake was concentrated to give ethyl 6-chloro-3-oxo-furo[2,3-b]pyridine-2-carboxylate 8.1 (9.4 g, 85.61% yield) as a white solid. ¹H NMR (400 MHZ, DMSO-d₆) δ 8.05 (d, J=8.0 Hz, 1H), 7.20 (d, J=8.0 Hz, 1H), 4.14 (q, J=6.8 Hz, 2H), 3.35-3.18 (m, 1H), 1.24 (t, J=8.0 Hz, 3H).

Step 2: To a solution of 8.1 (7.9 g, 32.69 mmol, 1 eq) was added HCl (54.77 g, 555.81 mmol, 53.70 mL, 37% purity, 17 eq). The mixture was stirred at 100° C. for 1 h. The reaction was quenched with H₂O (10 mL). The resulting mixture was extracted with EtOAc (100 mL×3). The combined organic phase was washed with brine (50 mL), dried over anhydrous Na₂SO₄, filtered and concentrated. The residue was purified by flash silica gel chromatography to give 6-chlorofuro[2,3-b]pyridin-3-one 8.2 (1.5 g, 27.06% yield) as a yellow solid. ¹H NMR (400 MHZ, DMSO-d₆) δ 8.19 (d, J=8.0 Hz, 1H), 7.36 (d, J=8.0 Hz, 1H), 4.95 (s, 2II).

Step 3: A mixture of ethyl 2-(triphenyl-25-phosphanylidene)acetate (3.70 g, 10.62 mmol, 1.2 eq), 8.2 (1.5 g, 8.85 mmol, 1 eq) in toluene (10 mL) was degassed and purged with N₂ for 3 times, and then the mixture was stirred at 130° C. for 16 h under N₂ atmosphere. The reaction mixture was concentrated directly. The residue was purified by flash silica gel chromatography to give ethyl 2-(6-chlorofuro[2,3-b]pyridin-3-yl)acetate 8.3 (1.2 g, 56.60% yield) as a white solid. ¹H NMR (400 MHZ, DMSO-d₆) § 8.18 (d, J=8.0 Hz, 1H), 8.07 (s, 1H), 7.49 (d, J=8.0 Hz, 1H), 4.11 (q, J=8.0 Hz, 2H), 3.84 (s, 2H), 1.19 (t, J=8.0 Hz, 3H).

Step 4: To a solution of 8.3 (1.1 g, 4.59 mmol, 1 eq) in DMF (10 mL) was added dropwise t-BuOK (566.55 mg, 5.05 mmol, 1.1 eq) at 0° C. After addition, the mixture was stirred at this temperature for 0.5 h, and then prop-2-enamide (652.48 mg, 9.18 mmol, 2 eq) in DMF (5 mL) was added dropwise at 0° C. under N₂ atmosphere. The resulting mixture was stirred at 0° C. for 1 h. The mixture was poured into water (20 mL) and extracted with EtOAc (50 mL×3). The combined organic layers were washed with brine (50 mL×3), dried over Na₂SO₄, filtered and concentrated to give a residue. The crude product was triturated with PE: EtOAc (10:1) at 20° C. for 60 min and filtered, the cake was concentrated to give Int 8 (0.5 g, 41.16% yield) as a yellow solid. ¹H NMR (400 MHZ, DMSO-d₆) § 10.95 (s, 1H), 8.18 (d, J=8.0 Hz, 1H), 8.08 (s, 1H), 7.48 (d, J=8.0 Hz, 1H), 4.18 (dd, J=4.8, 12.0 Hz, 1H), 2.72-2.66 (m, 1H), 2.63-2.56 (m, 1H), 2.36-2.31 (m, 1H), 2.18-2.06 (m, 1H).

### Synthesis of 3-(6-bromo-4-fluorobenzo[d]isoxazol-3-yl)piperidine-2,6-dione (Int 9)

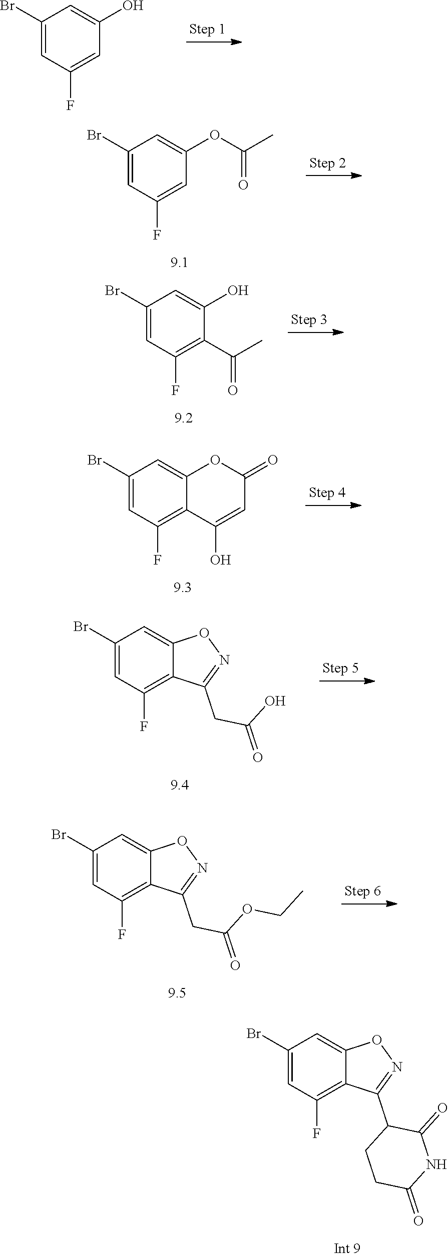

Step 1: To a solution of 3-bromo-5-fluoro-phenol (5 g, 26.18 mmol, 1 eq) in DCM (50 mL) was added pyridine (5.18 g, 65.45 mmol, 5.28 mL, 2.5 eq) and acetyl acetate (4.01 g, 39.27 mmol, 3.69 mL, 1.5 eq). The mixture was stirred at 25° C. for 16 h. The reaction mixture was concentrated. CuSO₄ (200 mL) was added to the residue. The resulting mixture was extracted with EtOAc (100 mL×3). The combined organic phase was washed with brine (100 mL), water (100 mL), dried over anhydrous Na₂SO₄, filtered and concentrated. The reaction mixture was concentrated directly, filtered and concentrated under reduced pressure to give (3-bromo-5-fluoro-phenyl)acetate 9.1 (6 g, 25.75 mmol, 98.35% yield) as a yellow solid. ¹H NMR (400 MHZ, CDCl₃) δ 7.15 (br d, J=8.0 Hz, 1H), 7.11 (d, J=1.6 Hz, 1H), 6.85 (br d, J=8.4 Hz, 1H), 2.31 (s, 3H).

Step 2: A solution of 9.1 (1 g, 4.29 mmol, 1 eq) in TfOH (2 g, 13.33 mmol, 1.18 mL, 3.11 eq) was stirred at 60° C. for 3 h. The reaction mixture was quenched with cold water (10 mL) drop-wise. The mixture was poured into cold water (20 mL) and extracted with CH₂Cl₂ (30 mL×3). The combined organic phase was washed with water (30 mL), dried over anhydrous Na₂SO₄, filtered and concentrated. The residue was purified by flash silica gel chromatography to give 1-(4-bromo-2-fluoro-6-hydroxy-phenyl) ethanone 9.2 (898 mg, 3.85 mmol, 89.80% yield) as a white solid. ¹H NMR (400 MHZ, CDCl₃) § 12.89 (s, 1H), 7.00 (s, 1H), 6.83 (dd, J=1.6, 12.0 Hz, 1H), 2.68 (d, J=7.6 Hz, 3H).

Step 3: To a solution of 9.2 (2 g, 8.58 mmol, 1 eq) in toluene (20 mL) was added NaH (1.03 g, 25.75 mmol, 190.96 μL, 60% purity, 3 eq) at 0° C. After addition, the mixture was stirred at 0° C. for 30 min, and then diethyl carbonate (2.03 g, 17.16 mmol, 2.08 mL, 2 eq) in toluene (20 mL) was added dropwise at 20° C. The mixture was stirred at 120° C. for 16 h. The reaction mixture was quenched with water (20 mL) at 0° C. After adjusting pH ˜ 5 with aq. HCl (1 M), the reaction mixture was filtered. The cake was concentrated to give 7-bromo-5-fluoro-4-hydroxy-2H-chromen-2-one 9.3 (800 mg, 3.09 mmol, 35.99% yield) as a white solid. ¹H NMR (400 MHZ, DMSO-d₆) δ 7.55 (t, J=1.6 Hz, 1H), 7.50 (dd, J=1.6, 12.0 Hz, 1H), 5.60 (s, 1H).

Step 4: To a solution of hydroxylamine (HCl salt) (643.86 mg, 9.27 mmol, 3 eq) in EtOH (30 mL) was added EtONa (630.51 mg, 9.27 mmol, 3 eq) at 0° C. Then 7-bromo-5-fluoro-4-hydroxy-chromen-2-one (800 mg, 3.09 mmol, 1 eq) was added at 0° C. The mixture was stirred at 80° C. for 16 h. The reaction mixture was concentrated directly. The residue was purified by flash silica gel chromatography to give 2-(6-bromo-4-fluorobenzo[d]isoxazol-3-yl) acetic acid 9.4 (357 mg, 1.30 mmol, 42.18% yield) as a yellow solid. ¹HNMR (400 MHZ, CDCl₃) δ 7.62 (s, 1H), 7.18 (d, J=8.4 Hz, 1H), 4.15 (s, 2H).

Step 5: To a solution of 9.4 (357 mg, 1.30 mmol, 1 eq) in EtOH (5 mL) was added H₂SO₄ (255.54 mg, 2.61 mmol, 138.88 μL, 2 eq) in EtOH (5 mL). The mixture was stirred at 70° C. for 8 h. The reaction mixture was concentrated directly. The resulting mixture was extracted with EtOAc (20 mL×3). The combined organic phase was washed with brine (20 mL), dried over anhydrous Na₂SO₄, filtered and concentrated to give ethyl ethyl 2-(6-bromo-4-fluorobenzo[d]isoxazol-3-yl)acetate 9.5 (374 mg, 1.24 mmol, 95.03% yield) as a dark brown liquid. ¹H NMR (400 MHZ, DMSO-d₆) δ 8.05 (s, 1H), 7.58 (d, J=8.4 Hz, 1H), 4.23 (s, 2H), 4.15 (q, J=7.2 Hz, 2H), 1.18 (t, J=7.2 Hz, 3H).

Step 6: To a solution of 9.5 (1 g, 3.31 mmol, 1 eq) in DMF (10 mL) was added t-BuOK (408.58 mg, 3.64 mmol, 1.1 eq) at 0° C. After addition, the mixture was stirred at 0° C. for 30 min, and then prop-2-enamide (705.85 mg, 9.93 mmol, 685.29 μL, 3 eq) was added at 0° C. under N₂. The mixture was stirred at 0° C. for 1 h. The mixture was poured into water (50 mL) and extracted with EtOAc (50 mL×3). The combined organic layers were washed with brine (50 mL), dried over Na₂SO₄, filtered and concentrated to give a residue. The crude product was triturated with DCM (5 mL) at 20° C. for 1 h. And then filtered, the cake was concentrated to give ethyl 3-(6-bromo-4-fluorobenzo[d]isoxazol-3-yl)piperidine-2,6-dione Int 9 (158 mg, 483.02 μmol, 14.59% yield) as a yellow solid. ¹H NMR (400 MHZ, DMSO-d₆) δ 11.19 (br s, 1H), 8.09 (d, J=1.2 Hz, 1H), 7.61 (dd, J=1.2, 8.4 Hz, 1H), 4.67-4.61 (m, 1H), 2.90-2.80 (m, 1H), 2.69-2.64 (m, 1H), 2.36 (br d, J=1.2 Hz, 1H), 2.36-2.31 (m, 1H), 2.25-2.18 (m, 1H).

### Synthesis of 3-(6-bromo-5-chlorobenzofuran-3-yl)piperidine-2,6-dione (Int 10)

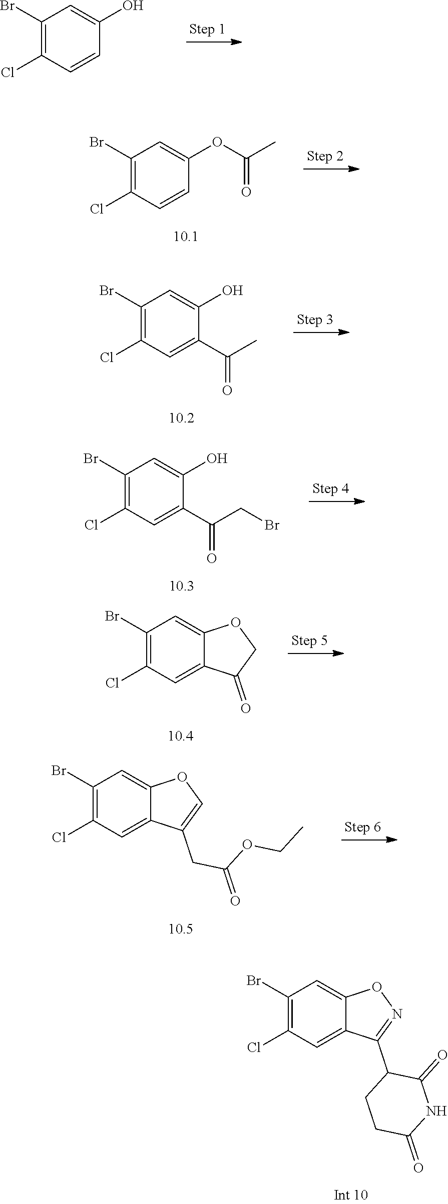

Step 1: To a solution of 3-bromo-4-chlorophenol (25 g, 120.51 mmol, 1 eq) and acetyl acetate (18.45 g, 180.76 mmol, 1.5 eq) in DCM (250 mL) was added pyridine (23.83 g, 301.27 mmol, 2.5 eq). The mixture was stirred at 25° C. for 12 h. The reaction mixture was concentrated. DCM (300 mL) was added to the residue. The combined organic phase was washed with CuSO₄ (500 mL), dried over anhydrous Na₂SO₄, filtered and concentrated under reduced pressure to give 3-bromo-4-chlorophenyl acetate 10.1 (67.4 g, crude) as a brown oil. ¹H NMR (400 MHZ, DMSO-d₆) δ 7.71-7.63 (m, 2H), 7.24-7.21 (m, 1H), 2.27 (s, 3H).

Step 2: To a solution of 10.1 (30 g, 120.25 mmol, 1 eq) was added TfOH (90.23 g, 601.23 mmol, 5 eq) at 0° C. The mixture was stirred at 60° C. for 3 h. The residue was poured into cold water (300 mL). The aqueous phase was extracted with CH₂Cl₂ (200 mL×3). The combined organic phase was washed with brine (800 mL), dried with anhydrous Na₂SO₄, filtered and concentrated under reduced pressure to give 1-(4-bromo-5-chloro-2-hydroxyphenyl) ethan-1-one 10.2 (80.3 g, crude) as a brown solid. ¹H NMR (400 MHZ, CDCl₃) δ 12.08 (s, 1H), 7.75 (s, 1H), 7.30-7.23 (m, 1H), 2.59 (s, 3H).

Step 3: To a solution of 10.2 (30 g, 120.25 mmol, 1 eq) in CHCl₃ (150 mL) and EtOAc (150 mL) was added CuBr₂ (32.23 g, 144.30 mmol, 1.2 eq) under N₂ at 25° C. The mixture was stirred at 100° C. for 16 h. The reaction mixture was filtered and concentrated under reduced pressure to give 2-bromo-1-(4-bromo-5-chloro-2-hydroxyphenyl) ethan-1-one 10.3 (98 g, crude) as a green solid. ¹H NMR (400 MHZ, CDCl₃) δ 12.14-11.91 (m, 1H), 11.50 (s, 1H), 11.22 (s, 1H), 7.74-7.68 (m, 1H), 7.23-7.17 (m, 1H), 4.29 (s, 2H).

Step 4: To a solution of 10.3 (27 g, 82.22 mmol, 1 eq) in DCM (270 mL) was added Et₃N(9.98 g, 98.66 mmol, 1.2 eq) at 0° C. under N₂ atmosphere. The mixture was stirred at 25° C. for 2 h. The reaction mixture was poured into saturated NH₄Cl aqueous solution (150 mL) and extracted with DCM (100 mL×3). The combined organic layer was washed with brine (500 mL), dried over Na₂SO₄, filtered and concentrated. The residue was purified by flash silica gel chromatography and concentrated under reduced pressure to give 6-bromo-5-chlorobenzofuran-3 (2H)-one 10.4 (16.5 g, 64.87% yield) as a yellow solid. H NMR (400 MHZ, CDCl₃) δ 7.74 (s, 1H), 7.51 (s, 1H), 4.67 (s, 2H).

Step 5: To a solution of 10.4 (16.5 g, 66.67 mmol, 1 eq) in toluene (20 mL) was added ethyl 2-(triphenyl-phosphanylidene)acetate (27.87 g, 80.01 mmol, 1.2 eq). The mixture was stirred at 130° C. for 16 h. The reaction mixture was concentrated directly. The residue was purified by flash silica gel chromatography and concentrated under reduced pressure to give ethyl 2-(6-bromo-5-chlorobenzofuran-3-yl) 10.5 acetate (10.2 g, 48.17% yield) as a yellow solid. ¹H NMR (400 MHZ, CDCl₃) δ 7.78 (s, 1H), 7.69 (s, 1H), 7.64 (s, 1 H), 4.27-4.15 (m, 2H), 3.66 (s, 2H), 1.29 (t, J=8.0 Hz, 3H).

Step 6: To a solution of 10.5 (2 g, 6.30 mmol, 1 eq) and t-BuOK (848.05 mg, 7.56 mmol, 1.2 eq) in DMF (20 mL) was added acrylamide (1.34 g, 18.89 mmol, 3 eq) and purged with N₂ for 3 times, and then the mixture was stirred at 0° C. for 0.5 h under N₂ atmosphere. The reaction mixture was quenched by H₂O (30 mL). The resulting mixture was extracted with EtOAc (80 mL×2). The combined organic phase was washed with brine (60 mL), dried over anhydrous Na₂SO₄, filtered and concentrated. The crude product was triturated with petroleum ether:ethyl acetate=5:1 (10 mL) at 20° C. for 40 min. The mixture was filtered and concentrated under reduced pressure to give 3-(6-bromo-5-chlorobenzofuran-3-yl) piperidine-2,6-dione Int 10 (1.63 g, 75.55% yield) as a light yellow solid. ¹H NMR (400 MHz, DMSO-d₆) δ 10.94-10.87 (m, 1H), 8.14 (s, 1H), 8.04-8.00 (m, 1H), 7.97-7.93 (m, 1H), 4.21-4.10 (m, 1H), 2.78-2.65 (m, 1H), 2.62-2.63 (m, 1H), 2.40-2.29 (m, 1H), 2.14-2.02 (m, 1H).

### Synthesis of 3-(6-bromofuro[3,2-b]pyridin-3-yl)piperidine-2,6-dione (Int 11)

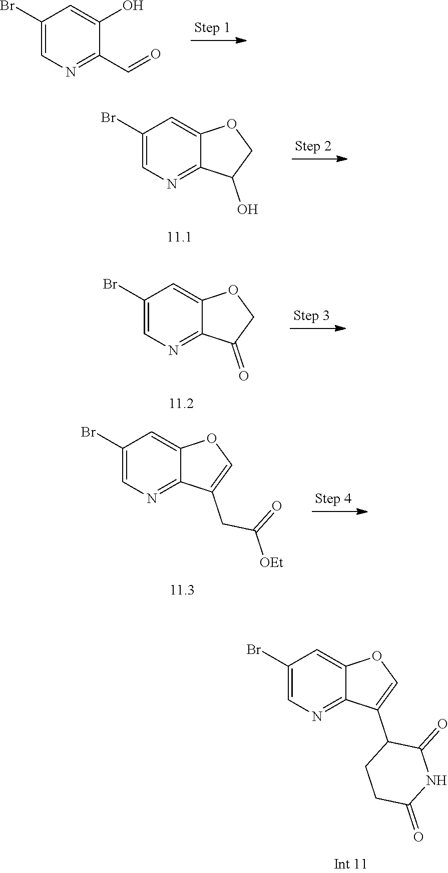 [SMILES: O=Cc1ncc(Br)cc1O ; OC1COc2cc(Br)cnc21 ; O=C1COc2cc(Br)cnc21 ; CCOC(=O)Cc1coc2cc(Br)cnc12 ; O=C1CCC(c2coc3cc(Br)cnc23)C(=O)N1]

Step 1: To a solution of 5-bromo-3-hydroxy-pyridine-2-carbaldehyde (5 g, 24.75 mmol, 1 eq) in DMSO (50 mL) was added trimethylsulfoxonium iodide (6.54 g, 29.70 mmol, 1.2 eq) and t-BuOK (3.33 g, 29.70 mmol, 1.2 eq). The mixture was stirred at 20° C. for 1.5 h. The mixture was poured into water (50 mL) and extracted with EtOAc (50 mL×3). The combined organic layers were washed with brine (50 mL), dried over Na₂SO₄, filtered and concentrated to give a residue. The residue was purified by column chromatography to give 6-bromo-2,3-dihydrofuro[3,2-b]pyridin-3-ol 11.1 (2 g, 9.26 mmol, 37.40% yield) as a yellow solid. ¹H NMR (400 MHZ, DMSO-d₆) δ 8.21 (d, J=2.0 Hz, 1H), 7.62 (d, J=1.6 Hz, 1H), 5.97 (d, J=6.0 Hz., 1H), 5.16-5.12 (m, 1H), 4.70-4.65 (m, 1H), 4.37-4.34 (m, 1H).

Step 2: To a solution of 11.1 (2 g, 9.26 mmol, 1 eq) in DCM (100 mL) was added Dess-Martin (7.85 g, 18.52 mmol, 5.73 mL, 2 eq). The mixture was stirred at 20° C. for 3 h. The mixture was poured into water (50 mL) and extracted with DCM (50 mL×2). The combined organic layers were washed with brine (50 mL×3), dried over Na₂SO₄, filtered and concentrated to give a residue. The residue was purified by column chromatography to give 6-bromofuro[3,2-b]pyridin-3-one 11.2 (260 mg, 1.21 mmol, 13.12% yield) as a yellow solid. ¹H NMR (400 MHZ, DMSO-d₆) δ 8.61 (d, J=2.0 Hz, 1H), 8.29 (d, J=1.6 Hz, 1H), 4.92 (s, 2H).

Step 3: A mixture of 11.2 (1.4 g, 6.54 mmol, 1 eq) ethyl 2-(triphenyl-25-phosphanylidene)acetate (2.73 g, 7.85 mmol, 1.2 eq) in toluene (20 mL) was degassed and purged with N₂ for 3 times, and then the mixture was stirred at 130° C. for 16 h under N₂ atmosphere. The mixture was poured into water (50 mL) and extracted with EtOAc (50 mL×3). The combined organic layers were washed with brine (50 mL), dried over Na₂SO₄, filtered and concentrated to give a residue. The residue was purified by column chromatography to give ethyl 2-(6-bromofuro[3,2-b]pyridin-3-yl)acetate 11.3 (1 g, 3.52 mmol, 53.81% yield) as a yellow solid. ¹H NMR (400 MHZ, DMSO-d₆) δ 8.62 (d, J=1.6 Hz, 1H), 8.44 (d, J=2.0 Hz, 1H), 8.26 (s, 1H), 4.13-4.07 (m, 2H), 3.80 (s, 2H), 1.20-1.16 (m, 3H).

Step 4: A mixture of ethyl 11.3 (500 mg, 1.76 mmol, 1 eq), prop-2-enamide (250.18 mg, 3.52 mmol, 242.89 μL, 2 eq), t-BuOK (217.23 mg, 1.94 mmol, 1.1 eq) in DMF (8 mL) was degassed and purged with N₂ for 3 times, and then the mixture was stirred at 0° C. for 1.5 h under N₂ atmosphere. The mixture was poured into water (20 mL) and extracted with EtOAc (50 mL×2). The combined organic layers were washed with brine (50 mL), dried over Na₂SO₄, filtered and concentrated to give a residue. The crude product was triturated with (PE: EtOAc=20:1) for 30 min. The mixture was filtered and the cake was concentrated to give 3-(6-bromofuro[3,2-b]pyridin-3-yl)piperidine-2,6-dione Int 11 (220 mg, 711.71 μmol, 40.44% yield) as a yellow solid. ¹H NMR (400 MHZ, DMSO-d₆) δ 10.94 (s, 1H), 8.61 (s, 1H), 8.46 (d, J=1.6 Hz, 1H), 8.29 (s, 1H), 4.18-4.14 (m, 1H), 2.80-2.72 (m, 1H), 2.60-2.59 (m, 1H), 2.44-2.40 (m, 1H), 2.14-2.13 (m, 1H).

### Synthesis of 3-(6-chlorofuro[3,2-c]pyridin-3-yl)piperidine-2,6-dione (Int 12)

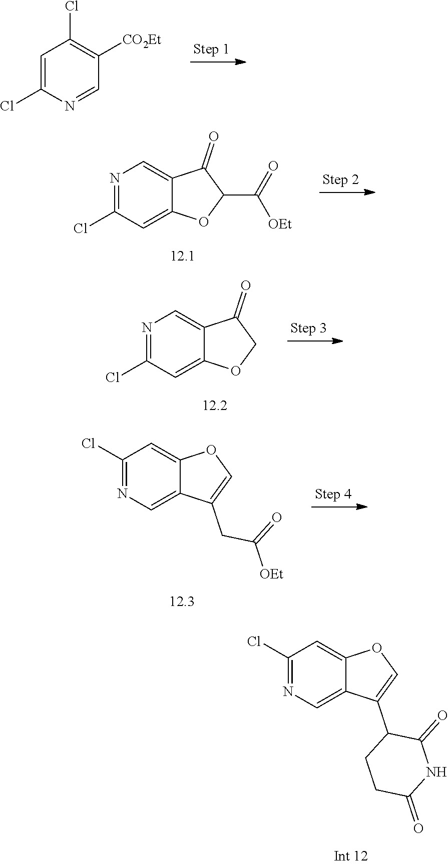 [SMILES: CCOC(=O)c1cnc(Cl)cc1Cl ; CCOC(=O)C1Oc2cc(Cl)ncc2C1=O ; O=C1COc2cc(Cl)ncc21 ; CCOC(=O)Cc1coc2cc(Cl)ncc12 ; O=C1CCC(c2coc3cc(Cl)ncc23)C(=O)N1]

Step 1: To a solution of NaH (9.09 g, 227.22 mmol, 60% purity, 2.5 eq) in DME (200 mL) was added ethyl 2-hydroxyacetate (23.65 g, 227.22 mmol, 21.90 mL, 2.5 eq) dropwise at 0° C. The reaction mixture was stirred at 0° C. for 0.5 h. And then ethyl 4,6-dichloropyridine-3-carboxylate (20 g, 90.89 mmol, 1 eq) in DME (100 mL) was added. The mixture was stirred at 75° C. for 2.5 h. The reaction mixture was quenched by H₂O (50 mL) drop-wise and concentrated. The crude product was triturated with EA: H₂O (90:150 mL) at 20° C. for 60 min. The mixture was filtered and the cake was concentrated to give 6-chloro-3-oxo-furo[3,2-c]pyridine-2-carboxylate 12.1 (21 g, 82.71 mmol, 91.00% yield) as a light yellow solid. MS: (ESI) m/z calculated for C₁₀H₈ClNO₄ [M+H]^{-242.0}, found 241.9.

Step 2: To a solution of ethyl 12.1 (21 g, 86.91 mmol, 1 eq) in HCl (300 mL). The mixture was stirred at 100° C. for 2 h. LCMS indicated desired MS was detected. Water (1000 mL) was added to the reaction mixture. The resulting mixture was extracted with EtOAc (600 mL×3). The combined organic phase dried over anhydrous Na₂SO₄, filtered and concentrated to give 6-chlorofuro[3,2-c]pyridin-3-one 12.2 (10.6 g, 61.01 mmol, 70.20% yield) as a red solid. MS: (ESI) m/z calculated for C₇H₄ClNO₂ [M+H]⁺170.0, found 170.0.

Step 3: A mixture of 12.2 (9.6 g, 56.62 mmol, 1 eq), ethyl 2-(triphenyl-25-phosphanylidene)acetate (23.67 g, 67.94 mmol, 1.2 eq) in toluene (300 mL) was degassed and purged with N₂ for 3 times, and then the mixture was stirred at 130° C. for 16 h under N₂ atmosphere. The reaction mixture was concentrated directly. The residue was purified by flash silica gel chromatography to give ethyl 2-(6-chlorofuro[3,2-c]pyridin-3-yl)acetate 12.3 (9.6 g, 38.33 mmol, 61.54% yield) as a light yellow solid. ¹HNMR (400 MHZ, DMSO-d₆) ¿ 8.75 (s, 1H), 8.05 (s, 1H), 7.87 (d, J=0.4 Hz, 1H), 4.13 (q, J=7.2 Hz, 2H), 3.89 (d, J=0.8 Hz, 2H), 1.22-1.18 (m, 3H).

Step 4: A mixture of ethyl 12.3 (9.6 g, 40.06 mmol, 1 eq), prop-2-enamide (8.54 g, 120.17 mmol, 8.29 mL, 3 eq), t-BuOK (6.74 g, 60.09 mmol, 1.5 eq) in DMF (100 mL) was degassed and purged with N₂ for 3 times, and then the mixture was stirred at 0° C. for 1.5 h under N₂ atmosphere. Water (500 mL) was added to the residue. The resulting mixture was extracted with EtOAc (300 mL×3). The combined organic phase was washed with brine (500 mL) dried over anhydrous Na₂SO₄, filtered and concentrated under reduced pressure. The reaction mixture was triturated with EtOAc (50 mL) for 60 min. The mixture was filtered and the cake was concentrated to give 3-(6-chlorofuro[3,2-c]pyridin-3-yl)piperidine-2,6-dione Int 12 (3.2 g, 9.35 mmol, 23.33% yield, 77.3% purity) as a yellow solid. ¹HNMR (400 MHz, DMSO-d₆) δ 10.97 (s, 1H), 8.75 (d, J=0.8 Hz, 1H), 8.07 (s, 1H), 7.90 (d, J=0.4 Hz, 1H), 4.24 (dd, J=4.8, 12.4 Hz, 1H), 2.80-2.70 (m, 1H), 2.64-2.57 (m, 1H), 2.38 (dd, J=4.4, 12.4 Hz, 1H), 2.18-2.11 (m, 1H).

### Synthesis of 3-(6-bromo-2-methylbenzofuran-3-yl)piperidine-2,6-dione (Int 13)

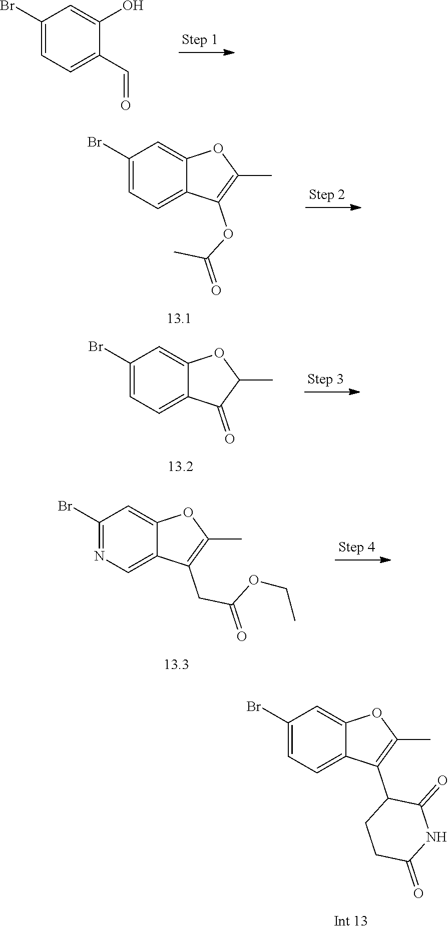

Step 1: To a solution of 4-bromo-2-hydroxy-benzaldehyde (10 g, 49.75 mmol, 1 eq), acetyl chloride (8.20 g, 104.47 mmol, 2.1 eq) and tributylphosphane (15.10 g, 74.62 mmol, 1.5 eq) in THF (300 mL) was added dropwise TEA (17.62 g, 174.11 mmol, 3.5 eq) at 0° C., The mixture was stirred at 25° C. for 5 h. The reaction mixture was concentrated. Water (500 mL) was added to the residue. The resulting mixture was extracted with EtOAc (300 mL×3). The combined organic phase was washed with brine (500 mL), dried over anhydrous Na₂SO₄, filtered and concentrated under reduced pressure to give a residue. The residue was purified by flash silica gel chromatography and concentrated under reduced pressure to give (6-bromo-2-methyl-benzofuran-3-yl)acetate 13.1 (12 g, crude) as a yellow solid. ¹HNMR (400 MHZ, CDCl₃) δ 7.55 (d, J=1.6 Hz, 1H), 7.35-7.31 (m, 1H), 7.18 (d, J=8.4 Hz, 1H), 2.38 (s, 3H), 2.35 (s, 3H).

Step 2: To a solution of 13.1 (12 g, 44.59 mmol, 1 eq) in MeOH (200 mL) was added HCl (1 M, 44.59 mL, 1 eq). The mixture was stirred at 75° C. for 8 h. The mixture was diluted with H₂O (200 mL) extracted with EtOAc (100 mL×3). The organic layer was washed with brine (200 mL), dried over Na₂SO₄, filtered and concentrated under reduced pressure to give a residue. The residue was purified by flash silica gel chromatography and concentrated under reduced pressure to give 6-bromo-2-methyl-benzofuran-3-one 13.2 (3 g, 29.63% yield) as a white solid. ¹H NMR (400 MHZ, CDCl₃) δ 7.52 (d, J=8.0 Hz, 1H), 7.22 (d, J=8.4 Hz, 1H), 7.00-6.85 (m, 1H), 4.66 (q, J=7.2 Hz, 1H), 1.53 (d, J=7.2 Hz, 3H).

Step 3: A mixture of 13.2 (2 g, 8.81 mmol, 1 eq), ethyl 2-(triphenyl-25-phosphanylidene)acetate (3.68 g, 10.57 mmol, 1.2 eq) in Tol. (40 mL) was degassed and purged with N₂ for 3 times, and then the mixture was stirred at 130° C. for 16 h under N₂ atmosphere. The mixture was concentrated under reduced pressure to give a residue. The residue was purified by flash silica gel chromatography and concentrated under reduced pressure to give ethyl 2-(6-bromo-2-methyl-benzofuran-3-yl)acetate 13.3 (0.95 g, 32.88% yield) as a yellow oil. ¹H NMR (400 MHZ, CDCl₃) δ 7.56 (s, 1H), 7.37-7.31 (m, 2H), 4.16 (q, J=7.2 Hz, 2H), 3.58 (s, 2H), 2.42 (s, 3H), 1.25 (t, J=7.2 Hz, 3H).

Step 4: A mixture of ethyl 13.3 (0.9 g, 3.03 mmol, 1 eq), prop-2-enamide (645.85 mg, 9.09 mmol, 3 eq), t-BuOK (509.81 mg, 4.54 mmol, 1.5 eq) in DMF (30 mL) was degassed and purged with N₂ for 3 times, and then the mixture was stirred at 0° C. for 1 h under N₂ atmosphere. The mixture was diluted with water (80 mL) extracted with EtOAc (50 mL×3). The organic layer was washed with brine (100 mL), dried over Na₂SO₄, filtered and concentrated under reduced pressure to give 3-(6-bromo-2-methyl-benzofuran-3-yl)piperidine-2,6-dione Int 13 (520 mg, crude) as a yellow solid. ¹HNMR (400 MHZ, DMSO-d₆) δ 10.93 (s, 1H), 7.80-7.75 (m, 1H), 7.38-7.31 (m, 2H), 4.15 (dd, J=12.6, 5.2 Hz, 1H), 2.84-2.74 (m, 1H), 2.60-2.52 (m, 1H), 2.39 (s, 3H), 2.35 (m, 1H), 1.98-1.89 (m, 1H).

### Synthesis of Boronic Esters

### Synthesis of 1-(2,3-dihydrobenzofuran-5-ylmethyl)-4-(4,4,5,5-tetramethyl-1,3,2-dioxaborolan-2-yl)pyrazole

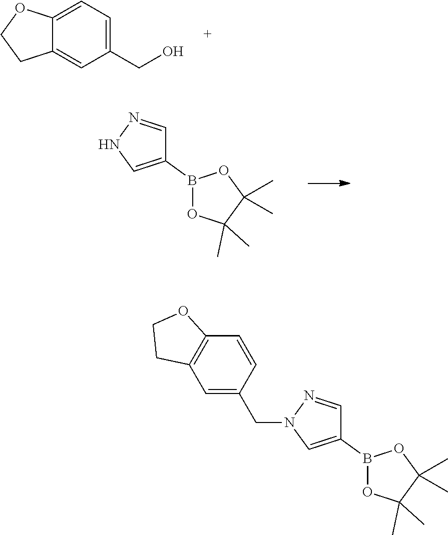 [SMILES: OCc1ccc2c(c1)CCO2 ; CC1(C)OB(c2cn[nH]c2)OC1(C)C ; CC1(C)OB(c2cnn(Cc3ccc4c(c3)CCO4)c2)OC1(C)C]

To a solution of 2,3-dihydrobenzofuran-5-ylmethanol (750 mg, 4.99 mmol, 1 eq), 4-(4,4,5,5-tetramethyl-1,3,2-dioxaborolan-2-yl)-1H-pyrazole (969.07 mg, 4.99 mmol, 1 eq) and PPh3 (1.31 g, 4.99 mmol, 1 eq) in THF (20 mL) was added DIAD (1.01 g, 4.99 mmol, 971.04 μL, 1 eq) at 0° C. Then the mixture was stirred at 20° C. for 16 hours. The reaction mixture was filtered directly and the cake was washed with EtOAc (10 mL). The filtrate was concentrated under reduced pressure to afford a residue. The residue was purified by column chromatography to afford 1-(2,3-dihydrobenzofuran-5-ylmethyl)-4-(4,4,5,5-tetramethyl-1,3,2-dioxaborolan-2-yl)pyrazole (806 mg, 4.16 mmol, 29.7% yield) as a colorless oil. LCMS: calc. for C₁₈H₂₃BN₂O₃: 326.2, found: [M+H]⁺327.2.

### Synthesis of N,N-dimethyl-4-[[4-(4,4,5,5-tetramethyl-1,3,2-dioxaborolan-2-yl)pyrazol-1-yl]methyl]benzenesulfonamide

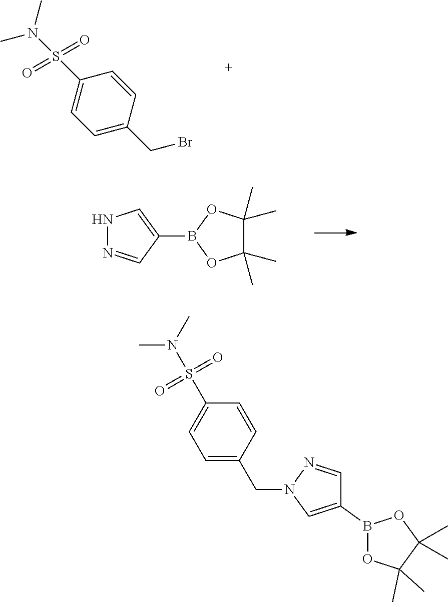

To a solution of 4-(bromomethyl)-N,N-dimethyl-benzenesulfonamide (50 mg, 179.75 μmol, 1 eq) and 4-(4,4,5,5-tetramethyl-1,3,2-dioxaborolan-2-yl)-1H-pyrazole (38.37 mg, 197.73 μmol, 1.1 eq) in ACN(5 mL) was added K₂CO₃ (49.69 mg, 359.50 μmol, 2 eq). The mixture was stirred at 80° C. for 4 h. The reaction liquid was filtered, and the filtrate was concentrated in vacuum to afford N,N-dimethyl-4-[[4-(4,4,5,5-tetramethyl-1,3,2-dioxaborolan-2-yl)pyrazol-1-yl]methyl]benzenesulfonamide (58 mg, 66.47% yield) as a colorless oil.

### Synthesis of 2-methyl-4-[[4-(4,4,5,5-tetramethyl-1,3,2-dioxaborolan-2-yl)pyrazol-1-yl]methyl]aniline and N-[2-methyl-4-[[4-(4,4,5,5-tetramethyl-1,3,2-dioxaborolan-2-yl)pyrazol-1-yl]methyl]phenyl]methanesulfonamide

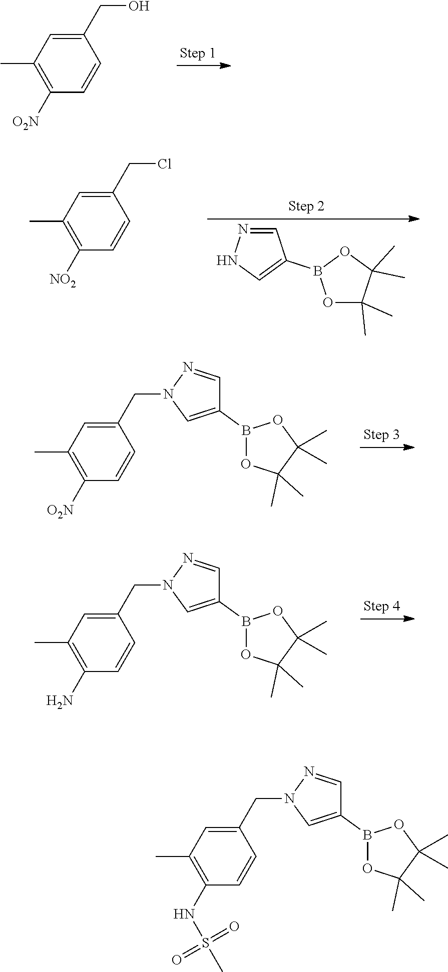

Step 1: A mixture of (3-methyl-4-nitro-phenyl) methanol (3.5 g, 20.94 mmol, 1 eq) in SOCl₂ (70.00 mL) was stirred at 80° C. for 16 hours. The reaction mixture was concentrated under reduced pressure to afford 4-(chloromethyl)-2-methyl-1-nitro-benzene (3.0 g, crude) as a yellow solid.

Step 2: To a solution of 4-(chloromethyl)-2-methyl-1-nitro-benzene (3.0 g, 16.16 mmol, 1 eq) and 4-(4,4,5,5-tetramethyl-1,3,2-dioxaborolan-2-yl)-1H-pyrazole (3.14 g, 16.16 mmol, 1 eq) in DMF (65 mL) was added K₂CO₃ (6.70 g, 48.49 mmol, 3 eq). The mixture was stirred at 60° C. for 16 hours. The reaction mixture was filtered directly and the filtrate was concentrated under reduced pressure to give a residue. The residue was purified by column chromatography to afford 1-[(3-methyl-4-nitro-phenyl)methyl]-4-(4,4,5,5-tetramethyl-1,3,2-dioxaborolan-2-yl)pyrazole (3.8 g, 8.01 mmol, 49.6% yield) as a yellow solid. LCMS: calc. for C₁₇H₂₂BN₃O₄: 343.2, found: [M+H]⁺344.0. ¹H NMR (400 MHZ, DMSO-d₆) δ 8.11 (s, 1H), 7.95-7.99 (m, 1H), 7.63 (s, 1H), 7.36 (s, 1H), 7.23 (dd, J=8.4, 1.6 Hz, 1H), 5.42 (s, 2H), 2.49 (s, 3H), 1.25 (s, 6H), 1.07 (s, 6H).

Step 3: To a solution of 1-[(3-methyl-4-nitro-phenyl)methyl]-4-(4,4,5,5-tetramethyl-1,3,2-dioxaborolan-2-yl)pyrazole (1 g, 2.91 mmol, 1 eq) in MeOH (100 mL) was added Pd/C (300 mg, 10% wt loading) under N₂ atmosphere. The suspension was degassed and purged with H₂ for 3 times, then the mixture was stirred under H₂ (15 psi) at 25° C. for 16 hours. The reaction mixture was filtered and concentrated under reduced pressure to afford 2-methyl-4-[[4-(4,4,5,5-tetramethyl-1,3,2-dioxaborolan-2-yl)pyrazol-1-yl]methyl]aniline (600 mg, crude) as a yellow oil. LCMS: calc. for C₁₇H₂₄BN₃O₂: 313.2, found: [M+H]⁺314.0

Step 4: To a solution of 2-methyl-4-[[4-(4,4,5,5-tetramethyl-1,3,2-dioxaborolan-2-yl)pyrazol-1-yl]methyl]aniline (500 mg, 1.60 mmol, 1 eq) and pyridine (378.83 mg, 4.79 mmol, 386.56 μL, 3 eq) in THF (20 mL) was added methylsulfonyl methanesulfonate (556.18 mg, 3.19 mmol, 2 eq) at 0° C., then the mixture was stirred at 25° C. for 16 hours. Crude LCMS showed 71% of desired product was found. The reaction mixture was concentrated under reduced pressure to give a residue. The residue was purified by column chromatography (SiO₂, petroleum ether/ethyl acetate=100/1 to 0/1) to afford N-[2-methyl-4-[[4-(4,4,5,5-tetramethyl-1,3,2-dioxaborolan-2-yl)pyrazol-1-yl]methyl]phenyl]methanesulfonamide (180 mg, 378.73 μmol, 23.7% yield) as a white solid. LCMS: calc. for C₁₈H₂₆BN₃O₄S: 391.2, found: [M+H]⁺391.9. ¹H NMR (400 MHZ, DMSO-d₆) δ 9.03 (s, 1H), 8.03 (s, 1H), 7.59 (s, 1H), 7.04-7.25 (m, 3H), 5.27 (s, 2H), 2.95 (s, 3H), 2.27 (s, 3H), 1.24 (s, 12H).

### Synthesis of 1-[1-(4-methoxyphenyl)-1-methyl-ethyl]-4-(4,4,5,5-tetramethyl-1,3,2-dioxaborolan-2-yl)pyrazole

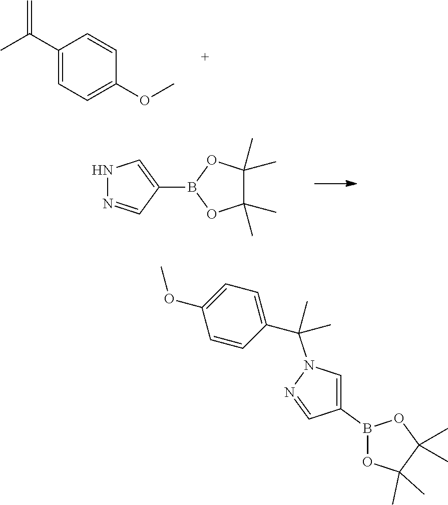

To a solution of 1-isopropenyl-4-methoxy-benzene (0.9 g, 6.07 mmol, 1 eq) and 4-(4,4,5,5-tetramethyl-1,3,2-dioxaborolan-2-yl)-1H-pyrazole (1.18 g, 6.07 mmol, 1 eq) in toluene (20 mL) was added I₂ (154.13 mg, 607.28 μmol, 0.1 eq). The mixture was heated to 110° C. for 16 hours. The reaction mixture was concentrated under reduced pressure to give a residue. The residue was purified by column chromatography to afford 1-[1-(4-methoxyphenyl)-1-methyl-ethyl]-4-(4,4,5,5-tetramethyl-1,3,2-dioxaborolan-2-yl)pyrazole (650 mg, 1.90 mmol, 31.27% yield) as colorless oil. LCMS: calc. for C₁₉H₂₇BN₂O₃: 342.2, found: [M+H]⁺343.0. ¹H NMR (400 MHZ, DMSO-d₆) § 7.93 (s, 1H), 7.63 (s, 1H), 6.97 (d, J=8.8 Hz, 2H), 6.84 (d, J=8.8 Hz, 2H), 3.72 (s, 3H), 1.89 (s, 6H), 1.25 (s, 12H).

### Synthesis of 4-[4-[[4-(4,4,5,5-tetramethyl-1,3,2-dioxaborolan-2-yl)pyrazol-1-yl]methyl]phenyl]morpholine

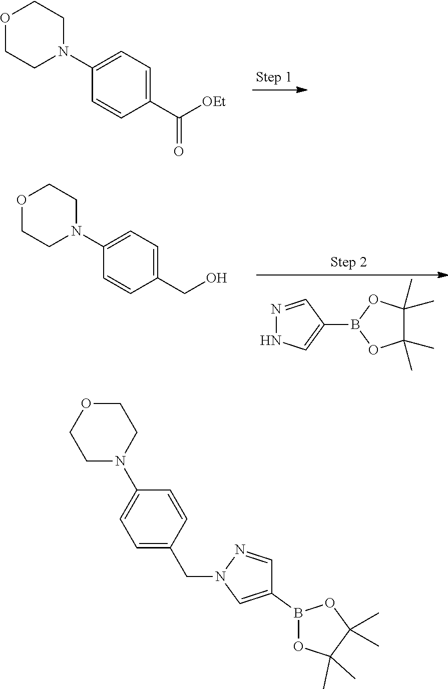 [SMILES: CCOC(=O)c1ccc(N2CCOCC2)cc1 ; OCc1ccc(N2CCOCC2)cc1 ; CC1(C)OB(c2cn[nH]c2)OC1(C)C ; CC1(C)OB(c2cnn(Cc3ccc(N4CCOCC4)cc3)c2)OC1(C)C]

Step 1: To a solution of ethyl 4-morpholinobenzoate (500 mg, 2.13 mmol, 1 eq) in THF (5 mL) was added LAH (201.65 mg, 5.31 mmol, 2.5 eq) at 0° C. The resulting mixture was stirred at 25° C. for 1 h. The reaction was quenched with H₂O (10 mL) and aq. NaOH (15% 5 mL) at 0° C. The resulting mixture was extracted with EtOAc (10 mL×3). The combined organic phase was washed with water (10 mL), brine (10 mL), dried over anhydrous Na₂SO₄, filtered and concentrated under reduced pressure to give (4-morpholinophenyl) methanol (400 mg, 94.48% yield) as a white solid. ¹H NMR (400 MHZ, DMSO-d₆) δ 7.17 (d, J=8.8 Hz, 2H), 6.87 (d, J=8.4 Hz, 2H), 4.38 (s, 2H), 3.74-3.71 (m, 4H), 3.07-3.03 (m, 4H).

Step 2: To a solution of (4-morpholinophenyl) methanol (200 mg, 1.03 mmol, 1 eq), 4-(4,4,5,5-tetramethyl-1,3,2-dioxaborolan-2-yl)-1H-pyrazole (200.82 mg, 1.03 mmol, 1 eq) and PPh3 (325.75 mg, 1.24 mmol, 1.2 eq) in THF (3 mL) was added di-tert-butyl diazene-1,2-dicarboxylate (285.98 mg, 1.24 mmol, 1.2 eq). The resulting mixture was stirred at 25° C. for 16 h. The reaction mixture was concentrated directly. The residue was purified by flash silica gel chromatography to give 4-[4-[[4-(4,4,5,5-tetramethyl-1,3,2-dioxaborolan-2-yl)pyrazol-1-yl]methyl]phenyl]morpholine (60 mg, crude) as a white solid.

### Synthesis of 1-[[4-(cyclopropylmethoxy)phenyl]methyl]-4-(4,4,5,5-tetramethyl-1,3,2-dioxaborolan-2-yl)pyrazole

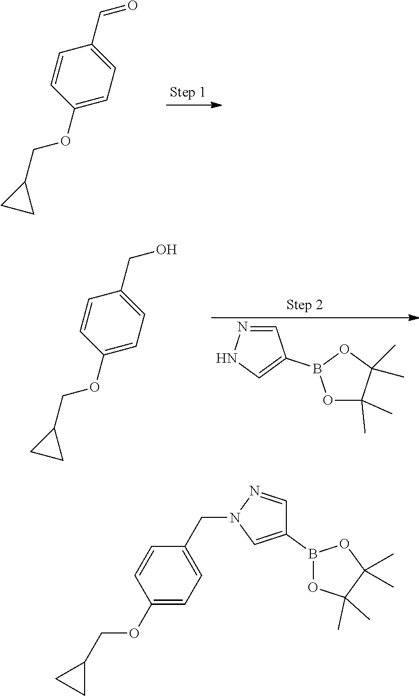 [SMILES: O=Cc1ccc(OCC2CC2)cc1 ; OCc1ccc(OCC2CC2)cc1 ; CC1(C)OB(c2cn[nH]c2)OC1(C)C ; CC1(C)OB(c2cnn(Cc3ccc(OCC4CC4)cc3)c2)OC1(C)C]

Step 1: To a solution of 4-(cyclopropylmethoxy)benzaldehyde (200 mg, 1.14 mmol, 1 eq) in EtOH (10 mL) was added NaBH₄ (50 mg, 1.32 mmol, 1.16 eq) slowly at 0° C., and stirred at 20° C. for 1 hour. A resulting yellow solution was formed. The excess NaBH₄ was quenched with saturated aq. NH₄Cl (10 mL) solution. The mixture was extracted with ethyl acetate (20 mL×3). The combined organic layers were washed with brine (20 mL), dried over anhydrous Na₂SO₄ and concentrated under reduced pressure to afford [4-(cyclopropylmethoxy)phenyl]methanol (178 mg, crude) as a white solid, which was used in the next step directly. ¹H NMR (400 MHZ, DMSO-d₆) δ 7.15-7.25 (m, 2H), 6.80-6.90 (m, 2H), 5.01 (t, J=5.6 Hz, 1H), 4.40 (d, J=5.6 Hz, 2H), 3.77 (d, J=6.8 Hz, 2H), 1.10-1.25 (m, 1H), 0.49-0.60 (m, 2H), 0.25-0.32 (m, 2H).

Step 2: To a solution of [4-(cyclopropylmethoxy)phenyl]methanol (1.9 g, 10.66 mmol, 1 eq), 4-(4,4,5,5-tetramethyl-1,3,2-dioxaborolan-2-yl)-1H-pyrazole (2.07 g, 10.66 mmol, 1 eq) and PPh3 (2.80 g, 10.66 mmol, 1 eq) in THF (20 mL) was added DIAD (2.16 g, 10.66 mmol, 2.07 mL, 1 eq) at 0° C., and then the mixture was stirred at 20° C. for 16 hours. The resulting yellow reaction mixture was concentrated under reduced pressure to give a residue. The residue was purified by column chromatography to afford 1-[[4-(cyclopropylmethoxy)phenyl]methyl]-4-(4,4,5,5-tetramethyl-1,3,2-dioxaborolan-2-yl)pyrazole (1.32 g, 3.13 mmol, 29.4% yield) as colorless oil. LCMS: calc. for C₂₀H₂₇BN₂O₃: 354.2, found: [M+H]⁺354.8. ¹H NMR (400 MHZ, DMSO-d₆) δ 7.96 (s, 1H), 7.58 (s, 1H), 7.20 (d, J=8.4 Hz, 2H), 6.87 (d, J=8.8 Hz, 2H), 5.23 (s, 2H), 3.78 (d, J=6.8 Hz, 2H), 1.99 (s, 1H), 1.23 (s, 12H), 0.53-0.58 (m, 2H), 0.28-0.32 (m, 2H).

### Synthesis of 4-(4,4,5,5-tetramethyl-1,3,2-dioxaborolan-2-yl)-1-[[4-(trideuteriomethoxy)phenyl]methyl]pyrazole

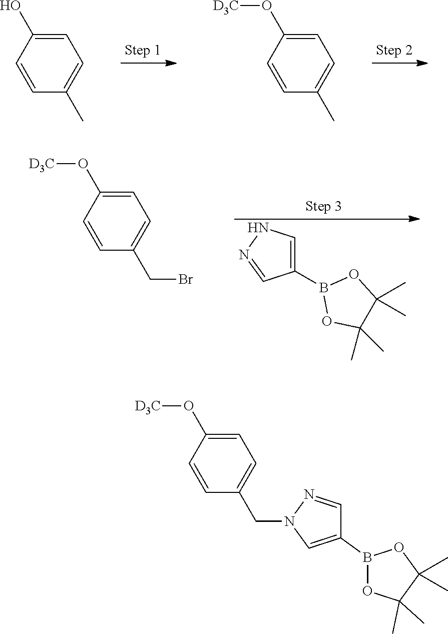

Step 1: The p-cresol (1 g, 9.25 mmol, 1 eq) was dissolved in THF (6 mL), the reaction mixture was cooled with ice water to 0° C., NaHl (739.73 mg, 18.49 mmol, 60% purity, 2 eq) was added. After stirring for 5 minutes, CD₃I (1.97 g, 13.87 mmol, 1.5 eq) was added. The mixture was stirred at 25° C. for 16 h. The reaction mixture was concentrated. Water (20 mL) was added to the residue. The resulting mixture was extracted with ethyl acetate (20 mL×3). The combined organic phase was washed with brine (20 mL×2), dried over anhydrous Na₂SO₄, filtered and concentrated to give 1-methyl-4-(trideuteriomethoxy)benzene (1 g, 86.38% yield) as a yellow oil. ¹H NMR (400 MHZ, CDCl₃) δ 7.11 (d, J=8.4 Hz, 2H), 6.82 (d, J=8.4 Hz, 2H), 2.31 (s, 3H).

Step 2: To a suspension of 1-methyl-4-(trideuteriomethoxy)benzene (1 g, 7.99 mmol, 1 eq) in CCl₄ (10 mL) were added AIBN(26.23 mg, 159.77 μmol, 0.02 eq) and NBS (1.14 g, 6.39 mmol, 0.8 eq) at 25° C. The reaction mixture was stirred at 80° C. for 3 h. The reaction mixture was concentrated. Water (30 mL) was added to the residue. The resulting mixture was extracted with DCM (20 mL×3). The combined organic phase was washed with brine (30 mL), dried over anhydrous Na₂SO₄, filtered and concentrated. The residue was purified by flash silica gel chromatography to give 1-(bromomethyl)-4-(trideuteriomethoxy)benzene (1 g, 49.07% yield, 80% purity) as a brown oil. ¹H NMR (400 MHZ, CDCl₃) δ 7.32 (br d, J=8.4 Hz, 2H), 6.86 (br d, J=8.4 Hz, 2H), 4.50 (s, 2H).

Step 3: To a solution of 1-(bromomethyl)-4-(trideuteriomethoxy)benzene (500 mg, 1.96 mmol, 80% purity, 1 eq) in ACN(8 mL) was added 4-(4,4,5,5-tetramethyl-1,3,2-dioxaborolan-2-yl)-1H-pyrazole (380.32 mg, 1.96 mmol, 1 eq) and K₂CO₃ (541.78 mg, 3.92 mmol, 2 eq) at 25° C. The mixture was stirred at 80° C. for 2 h. The reaction mixture was concentrated. Water (30 mL) was added to the residue. The resulting mixture was extracted with DCM (20 mL×3). The combined organic phase was washed with brine (30 mL), dried over anhydrous Na₂SO₄, filtered and concentrated. The residue was purified by flash silica gel chromatography to give 4-(4,4,5,5-tetramethyl-1,3,2-dioxaborolan-2-yl)-1-[[4-(trideuteriomethoxy)phenyl]methyl]pyrazole (200 mg, 22.52% yield, 70% purity) as a yellow oil. LCMS: calc. for C₁₇H₂OD₃BN₂O₃: 317.2, found: [M+H]⁺317.9.

### Synthesis of 1-[(4-methylsulfinylphenyl)methyl]-4-(4,4,5,5-tetramethyl-1,3,2-dioxaborolan-2-yl)pyrazole

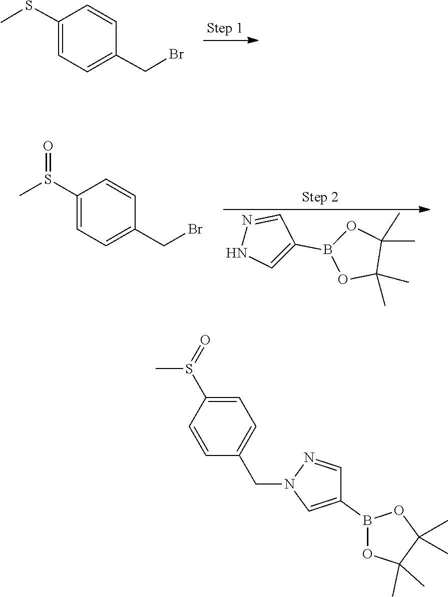

Step 1: To a mixture of 1-(bromomethyl)-4-methylsulfanyl-benzene (200 mg, 921.13 μmol, 1 eq) in HOAc (5 mL) was added H₂O₂ (1.2 g, 10.58 mmol, 30% purity, 11.49 eq) at 0° C. The reaction mixture was stirred at 25° C. for 2 h. The reaction mixture was concentrated. The reaction mixture was quenched by sat.Na₂S203 (30 mL) at 0° C. The mixture was extracted by EA (30 mL×3), the combined organic layers were washed by brine (50 mL), dried over Na₂SO₄, filtered and concentrated to give 1-(bromomethyl)-4-methylsulfinyl-benzene (250 mg, crude) as a light yellow liquid. LCMS: calc. for C₈H₉BrOS: 231.9, found: [M+H]⁺232.9.

Step 2: A mixture of 1-(bromomethyl)-4-methylsulfinyl-benzene (200 mg, 857.91 μmol, 1 eq), 4-(4,4,5,5-tetramethyl-1,3,2-dioxaborolan-2-yl)-1H-pyrazole (199.76 mg, 1.03 mmol, 1.2 eq) and K₂CO₃ (355.71 mg, 2.57 mmol, 3 eq) in DMF (5 mL) was degassed and purged with N₂ for 3 times, and then the mixture was stirred at 110° C. for 4 h under N₂ atmosphere. The reaction mixture was filtered. The filtrate was concentrated directly. The residue was purified by prep-HPLC to afford 1-[(4-methylsulfinylphenyl)methyl]-4-(4,4,5,5-tetramethyl-1,3,2-dioxaborolan-2-yl)pyrazole (40 mg, 6.73% yield) as a white solid. LCMS: calc. for C₁₇H₂₃BN₂O₃S: 346.1, found: [M+H]⁺347.1.

### Synthesis of diethyl(4-((4-(4,4,5,5-tetramethyl-1,3,2-dioxaborolan-2-yl)-1H-pyrazol-1-yl)methyl)phenyl)phosphonate

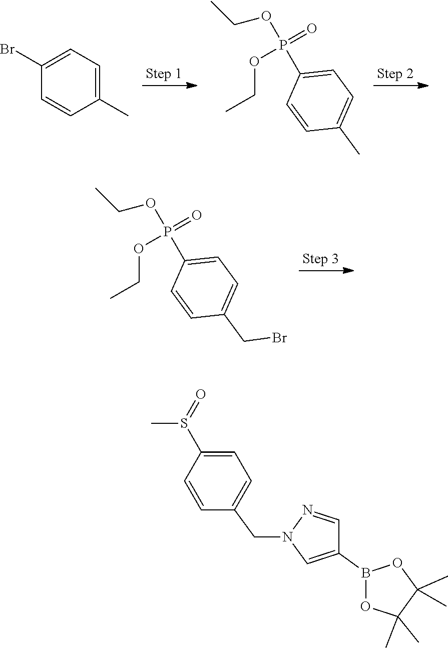

Step 1: A solution of 1-bromo-4-methyl-benzene (1.0 g, 5.8 mmol, 1 eq), 1-ethoxyphosphonoyloxyethane (968.9 mg, 7.0 mmol, 1.2 eq), Pd(PPh3)₄ (675.6 mg, 584.6 μmol, 0.1 eq), TEA (650.8 mg, 6.4 mmol, 1.1 eq) in toluene (15 mL) was degassed and purged with N₂ for 3 times, and then the mixture was stirred at 90° C. for 16 h under N₂ atmosphere. The mixture was diluted with H₂O (30 mL), extracted with EtOAc (20 mL×2). The organic layer was washed with brine (30 mL), dried over Na₂SO₄, filtered and concentrated. The residue was purified by flash silica gel chromatography to give 1-diethoxyphosphoryl-4-methyl-benzene (750 mg, 53.05% yield) as a black brown oil. ¹H NMR (400 MHZ, CDCl₃) δ 7.82-7.55 (m, 2H), 7.32-7.18 (m, 2H), 4.22-3.89 (m, 4H), 2.50-2.31 (m, 3H), 1.45-1.06 (m, 6H).

Step 2: A solution of 1-diethoxyphosphoryl-4-methyl-benzene (650.0 mg, 2.8 mmol, 1 eq), NBS (456.2 mg, 2.6 mmol, 0.9 eq) and AIBN(46.7 mg, 284.8 μmol, 0.1 eq) in MeCN(10 mL) was degassed and purged with N₂ for 3 times, and then the mixture was stirred at 80° C. for 2 h under N₂ atmosphere. The mixture was diluted with H₂O (20 mL), then was extracted with EtOAc (20 mL×2). The organic layer was washed with brine (30 mL), dried over Na₂SO₄, filtered and concentrated. The residue was purified by flash silica gel chromatography to give 1-(bromomethyl)-4-diethoxyphosphoryl-benzene (450 mg, 38.93% yield) as a colorless oil. ¹H NMR (400 MHZ, DMSO-d₆) δ 7.68-7.61 (m, 2H), 7.57-7.51 (m, 2H), 4.74-4.66 (m, 2H), 3.98-3.91 (m, 4H), 1.18-1.14 (m, 6H).

Step 3: To a solution of 1-(bromomethyl)-4-diethoxyphosphoryl-benzene (150.0 mg, 488.4 μmol, 1 eq) and 4-(4,4,5,5-tetramethyl-1,3,2-dioxaborolan-2-yl)-1H-pyrazole (85.3 mg, 439.5 μmol, 0.9 eq) in ACN(10 mL) was added K₂CO₃ (135.0 mg, 976.8 μmol, 2 eq). The mixture was stirred at 80° C. for 16 h. The mixture was diluted with H₂O (20 mL), then was extracted with EtOAc (20 mL×2). The organic layer was washed with brine (30 mL), dried over Na₂SO₄, filtered and concentrated. The residue was purified by flash silica gel chromatography to give diethyl(4-((4-(4,4,5,5-tetramethyl-1,3,2-dioxaborolan-2-yl)-1H-pyrazol-1-yl)methyl)phenyl)phosphonate (160 mg, crude) as a white oil. LCMS calc. for C₂₀H₃₀BN₂O₅P: 420.2, found: [M+H]⁺420.9.

### Synthesis of 1-methyl-4-[6-[[4-(4,4,5,5-tetramethyl-1,3,2-dioxaborolan-2-yl)pyrazol-1-yl]methyl]-3-pyridyl]piperazine

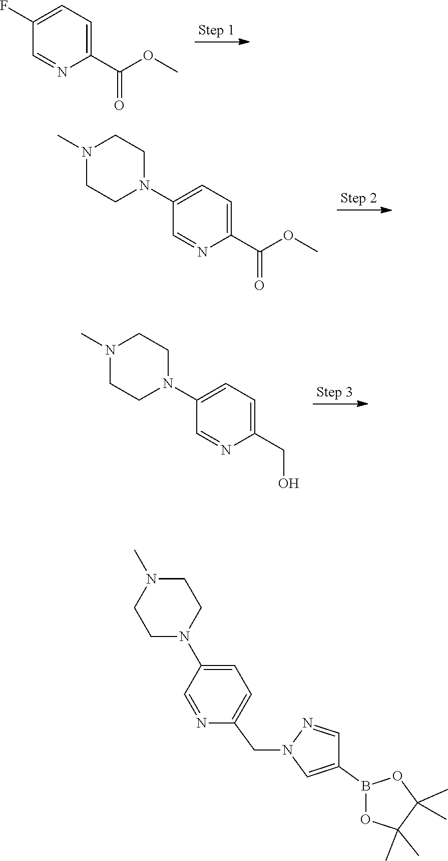

Step 1: A mixture of methyl 5-fluoropyridine-2-carboxylate (1 g, 6.45 mmol, 1 eq), 1-methylpiperazine-dihydrochloride (1.62 g, 7.74 mmol, 1.2 eq, HCl), K₂CO₃ (1.34 g, 9.67 mmol, 1.5 eq) in DMSO (10 mL) was degassed and purged with N₂ for 3 times, and then the mixture was stirred at 110° C. for 16 h under N₂ atmosphere. Water (60 mL) was added to the residue. The resulting mixture was extracted with DCM (50 mL×3). The combined organic phase was washed with brine (50 mL), dried over anhydrous Na₂SO₄, filtered and concentrated to give methyl 5-(4-methylpiperazin-1-yl)pyridine-2-carboxylate (1.2 g, 5.10 mmol, 79.12% yield) as a yellow solid. LCMS calc. for C₁₂H₁₇N₃O₂:235.1, found: [M+H]⁺236.1.

Step 2: To a mixture of methyl 5-(4-methylpiperazin-1-yl)pyridine-2-carboxylate (600 mg, 2.55 mmol, 1 eq) in THF (8 mL) was added NaBH₄ (320 mg, 8.46 mmol, 3.32 eq) and the reaction mixture was stirred at 50° C. for 2.5 h. The solution was cooled to 0° C., quenched with HCl (1M) (6 mL), water (20 mL) was added to the residue. The resulting mixture was extracted with ethyl acetate (20 mL×3), the combined organic phase was washed with brine (30 mL), dried over anhydrous Na₂SO₄, filtered and concentrated. The residue was purified by flash silica gel chromatography to give [5-(4-methylpiperazin-1-yl)-2-pyridyl]methanol (100 mg, 482.46 μmol, 18.92% yield) as a colorless oil. LCMS calc. for C₁₁H₁₇N₃O: 207.1, found: [M+H]⁺208.0.

Step 3: To a solution of [5-(4-methylpiperazin-1-yl)-2-pyridyl]methanol (100 mg, 482.46 μmol, 1 eq), 4-(4,4,5,5-tetramethyl-1,3,2-dioxaborolan-2-yl)-1H-pyrazole (93.62 mg, 482.46 μmol, 1 eq) and PPh3 (189.81 mg, 723.69 μmol, 1.5 eq) in THF (4 mL) was added DBAD (166.64 mg, 723.69 μmol, 1.5 eq) at 0° C. The reaction was stirred at 25° C. for 16 h. The reaction mixture was concentrated. The residue was purified by flash silica gel chromatography to give 1-methyl-4-[6-[4-(4,4,5,5-tetramethyl-1,3,2-dioxaborolan-2-yl)pyrazol-1-yl]methyl]-3-pyridyl]piperazine (50 mg, 78.27 μmol, 16.22% yield, 60% purity) as a colorless oil. LCMS calc. for C₂₀H₃₀BN₅O₂: 383.2, found: [M+H]⁺384.2.

### Synthesis of N,N-dimethyl-5-[[4-(4,4,5,5-tetramethyl-1,3,2-dioxaborolan-2-yl)pyrazol-1-yl]methyl]pyridin-2-amine

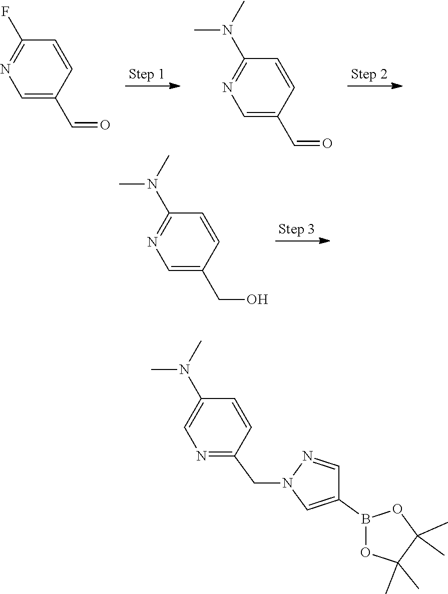

Step 1: To a solution of 6-fluoropyridine-3-carbaldehyde (2 g, 15.99 mmol, 1 eq), N-methylmethanamine (HCl salt) (1.56 g, 19.18 mmol, 1.2 eq) in DMSO (25 mL) was added K₂CO₃ (2.65 g, 19.18 mmol, 1.2 eq). The mixture was stirred at 110° C. for 2 h. The reaction mixture was added H₂O (50 mL), and then extracted with EtOAc (50 mL×3). The combined organic layers were washed with brine (100 mL), dried over Na₂SO₄, filtered and concentrated to give 6-(dimethylamino)pyridine-3-carbaldehyde (2 g, 11.99 mmol, 74.97% yield, 90% purity) as a white solid. ¹HNMR (400 MHZ, DMSO-d₆) δ 9.71 (s, 1H), 8.56 (d, J=2.0 Hz, 1H), 7.84 (dd, J=2.0, 8.8 Hz, 1H), 6.73 (d, J=8.8 Hz, 1H), 3.14 (s, 6H).

Step 2: To a solution of 6-(dimethylamino)pyridine-3-carbaldehyde (500 mg, 3.33 mmol, 1 eq) in MeOH (10 mL) was added NaBH₄ (0.290 g, 7.67 mmol, 2.30 eq) at 0° C. The mixture was stirred at 25° C. for 3 h. The reaction mixture was quenched by Sat. NH₄Cl (10 mL) at 0° C. and the reaction mixture was concentrated. The mixture was diluted with H₂O (30 mL) and extracted with DCM (45 mL×3). The combined with organic layers was concentrated to give residue. The residue was purified by flash silica gel chromatography to give [6-(dimethylamino)-3-pyridyl]methanol (400 mg, 2.59 mmol, 77.76% yield) as a yellow oil. ¹HNMR (400 MHZ, DMSO-d₆) δ 8.01 (d, J=2.0 Hz, 1H), 7.46 (dd, J=2.4, 8.4 Hz, 1H), 6.61 (d, J=8.8 Hz, 1H), 4.95 (t, J=5.6 Hz, 1H), 4.33 (d, J=5.6 Hz, 2H), 3.00 (s, 6H).

Step 3: A mixture of [6-(dimethylamino)-3-pyridyl]methanol (400 mg, 2.63 mmol, 1 eq), 4-(4,4,5,5-tetramethyl-1,3,2-dioxaborolan-2-yl)-1H-pyrazole (509.98 mg, 2.63 mmol, 1 eq), 2-(tributyl-25-phosphanylidene) acetonitrile (1.27 g, 5.26 mmol, 2 eq) in toluene (10 mL) was degassed and purged with N₂ for 3 times, and then the mixture was stirred at 110° C. for 16 h under N₂ atmosphere. The reaction mixture was concentrated directly. The residue was purified by prep-HPLC to give N,N-dimethyl-5-[[4-(4,4,5,5-tetramethyl-1,3,2-dioxaborolan-2-yl)pyrazol-1-yl]methyl]pyridin-2-amine (250 mg, 761.69 μmol, 28.98% yield) as a yellow oil.

### Synthesis of 4-[2-[[4-(4,4,5,5-tetramethyl-1,3,2-dioxaborolan-2-yl)pyrazol-1-yl]methyl]pyrimidin-5-yl]morpholine

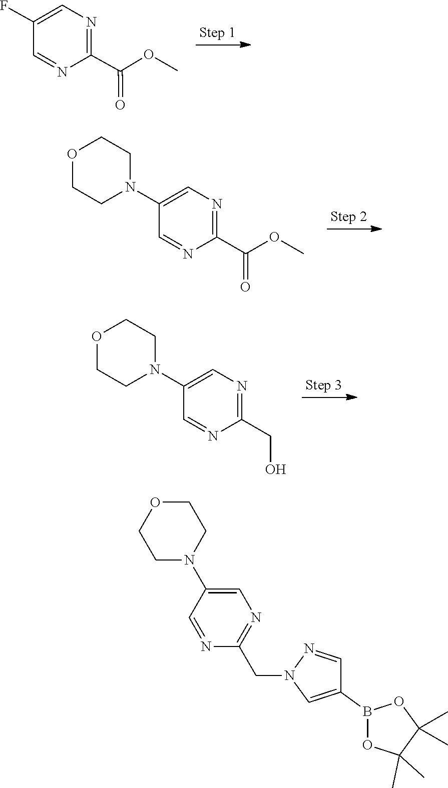

Step 1: To a solution of methyl 5-fluoropyrimidine-2-carboxylate (500 mg, 3.20 mmol, 1 eq), morpholine (418.54 mg, 4.80 mmol, 422.77 μL, 1.5 eq) in ACN(5 mL) was added K₂CO₃ (531.17 mg, 3.84 mmol, 1.2 eq), the mixture was stirred at 85° C. for 16 h. The reaction mixture was concentrated directly to give methyl 5-morpholinopyrimidine-2-carboxylate (500 mg, 69.93% yield) as a white solid.

Step 2: NaBH₄ (1.43 g, 37.80 mmol, 2.81 eq) was slowly added to methyl 5-morpholinopyrimidine-2-carboxylate (3 g, 13.44 mmol, 1 eq) in MeOH (30 mL) under nitrogen at 0° C., The reaction was refluxed at 25° C. for 2 h. The mixture was quenched with sat. NH₄C₁ (30 mL), then diluted with further water (50 mL), extracted with ethyl acetate (50 mL×3), The combined organic extracts were dried over sodium sulphate, filtered and concentrated. The residue was purified by flash silica gel chromatography to give (5-morpholinopyrimidin-2-yl) methanol (300 mg, 11.43% yield) as a white solid. ¹H NMR (400 MHz, DMSO-d₆) δ 8.46 (s, 2H), 5.10 (t, J=8.0 Hz, 1H), 4.49 (d, J=8.0 Hz, 2H), 3.80-3.66 (m, 4H), 3.25-3.16 (m, 4H).

Step 3: A mixture of (5-morpholinopyrimidin-2-yl) methanol (360 mg, 1.84 mmol, 1 eq), 4-(4,4,5,5-tetramethyl-1,3,2-dioxaborolan-2-yl)-1H-pyrazole (286.26 mg, 1.48 mmol, 0.8 eq), 2-(tributyl-phosphanylidene) acetonitrile (1.11 g, 4.61 mmol, 2.5 eq) in toluene (10 mL) was degassed and purged with N₂ for 3 times, and then the mixture was stirred at 110° C. for 16 h under N₂ atmosphere. The reaction mixture was concentrated directly. The residue was purified by flash silica gel chromatography to give 4-[2-[4-(4,4,5,5-tetramethyl-1,3,2-dioxaborolan-2-yl)pyrazol-1-yl]methyl]pyrimidin-5-yl]morpholine (600 mg, 63.98% yield) as a yellow liquid.

### Synthesis of 5-methyl-2-[[4-(4,4,5,5-tetramethyl-1,3,2-dioxaborolan-2-yl)pyrazol-1-yl]methyl]pyridine

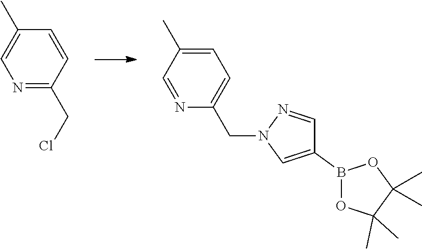

To a solution of 2-(chloromethyl)-5-methyl-pyridine (200 mg, 1.41 mmol, 1 eq), 4-(4,4,5,5-tetramethyl-1,3,2-dioxaborolan-2-yl)-1H-pyrazole (328.88 mg, 1.69 mmol, 1.2 eq) in ACN(10 mL) was added Cs₂CO₃ (920.41 mg, 2.82 mmol, 2 eq). The mixture was stirred at 80° C. for 3 h. The reaction mixture was concentrated. Water (20 mL) was added to the residue. The resulting mixture was extracted with EtOAc (20 mL×3). The combined organic phase was washed with water (20 mL), dried over anhydrous Na₂SO₄, filtered and concentrated. The residue was purified by flash silica gel chromatography under reduced pressure to give 5-methyl-2-[[4-(4,4,5,5-tetramethyl-1,3,2-dioxaborolan-2-yl)pyrazol-1-yl]methyl]pyridine (250 mg, 835.63 μmol, 59.16% yield) as a colorless oil.

### Synthesis of 4-cyclopentyl-2-((4-(4,4,5,5-tetramethyl-1,3,2-dioxaborolan-2-yl)-1H-pyrazol-1-yl)methyl)pyrimidine

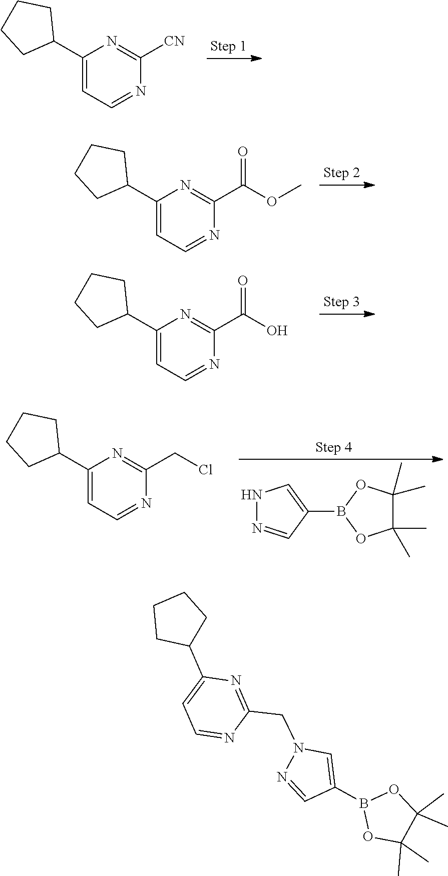

Step 1: A mixture of 5-cyclopentylpyrimidine-2-carbonitrile (300 mg, 1.73 mmol, 1 eq) in HCl/MeOH (10 mL, 4M) was degassed and purged with N₂ for 3 times, and then the mixture was stirred at 80° C. for 3 hr under N₂ atmosphere. Crude LCMS showed the starting material was consumed completely, and 52% of desired MS was observed. The reaction mixture was added to water (10 mL), the reaction mixture was adjusted pH˜7 with aq. NaHCO₃ and extracted with ethyl acetate (20 mL×2). The combined organic layers were concentrated and washed with brine (15 mL), dried over anhydrous Na₂SO₄ and concentrated under reduced pressure to afford methyl 5-cyclohexylpyridazine-3-carboxylate (820 mg, crude) as a yellow oil. LCMS: calc. for C₁₁H₁₄N₂O₂:206.1, found: [M+H]⁺207.0.

Step 2: To a solution of methyl 5-cyclopentylpyrimidine-2-carboxylate (700 mg, 3.39 mmol, 1 eq) in EtOH (10 mL) was added NaBH₄ (200 mg, 5.29 mmol, 1.56 eq) at 0° C. The mixture was stirred at 25° C. for 4 h under N₂. Crude LCMS showed 38% of desired product was found. The mixture was quenched with sat. aq. NH₄Cl (10 mL×3) at 0° C. The resulting mixture was extracted with ethyl acetate (20 mL×3). The combined organic phase was washed with brine (40 mL), dried over anhydrous sodium sulfate, filtered and concentrated under vacuum to afford (5-cyclopentylpyrimidin-2-yl) methanol (660 mg, crude) as a yellow oil. LCMS: calc. for C₁₀H₁₄N₂O: 178.1, found: [M+H]⁺179.0.

Step 3: A To a solution of (5-cyclopentylpyrimidin-2-yl) methanol (660 mg, crude) in DCM (2 mL) was added SOCl₂ (1.32 g, 11.11 mmol, 805.89 μL, 3 eq). The mixture was stirred at 25° C. for 3 h under N₂ atmosphere. Crude LCMS showed 34% of desired MS was observed. The mixture was filtered and concentrated to give a residue. The residue was purified by prep-TLC to give 2-(chloromethyl)-4-cyclopentylpyrimidine (250 mg, 564.38 μmol, 15.24% yield) as a yellow oil. ¹HNMR (400 MHZ, DMSO-d₆) δ 8.84 (d, J=5.2 Hz, 1H), 7.55 (d, J=5.2 Hz, 1H), 4.82 (s, 2H), 2.33-2.38 (m, 2H), 2.17-2.27 (m, 1H), 1.72-1.83 (m, 2H), 1.56-1.71 (m, 4H). LCMS: calc. for C₁₀H₁₃ClN₂:196.1, found: [M+H]⁺197.1.

Step 4: To a solution of 4-(4,4,5,5-tetramethyl-1,3,2-dioxaborolan-2-yl)-1H-pyrazole (295.98 mg, 1.53 mmol, 1.2 eq) and 2-(chloromethyl)-4-cyclopentylpyrimidine (250 mg, 1.27 mmol, 1 eq) in DMF (3 mL) was added Cs₂CO₃ (828.31 mg, 2.54 mmol, 2 eq). The mixture was stirred at 25° C. for 2 h under N₂ atmosphere. Crude LCMS showed 11% of desired product was found. The reaction mixture was poured into water (20 mL) and extracted with ethyl acetate (20 mL×3). The combined organic layer was washed with brine (50 mL), dried over anhydrous Na₂SO₄ and concentrated under reduced pressure to afford 5-cyclopentyl-2-[[4-(4,4,5,5-tetramethyl-1,3,2-dioxaborolan-2-yl)pyrazol-1-yl]methyl]pyrimidine (360 mg, crude) as a yellow oil. LCMS: calc. for C₁₉H₂₇BN₄O₂: 354.2, found: [M+H]⁺355.2.

### Synthesis of 2-tetrahydropyran-2-yl-5-[[4-(4,4,5,5-tetramethyl-1,3,2-dioxaborolan-2-yl)pyrazol-1-yl]methyl]pyrazine

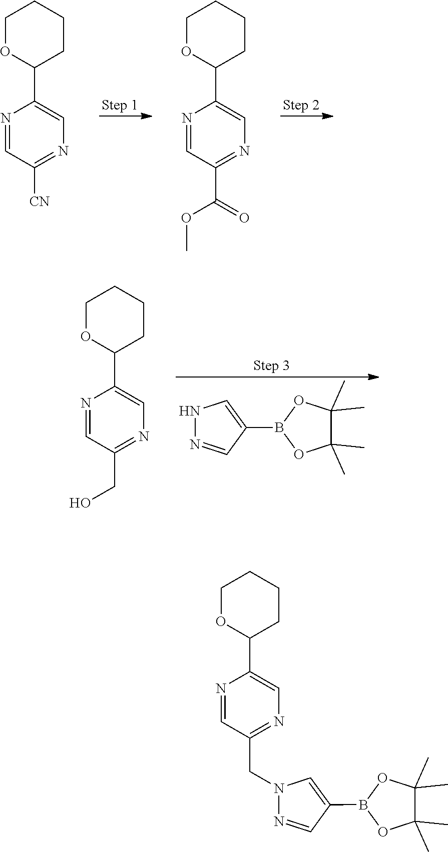 [SMILES: N#Cc1cnc(C2CCCCO2)cn1 ; COC(=O)c1cnc(C2CCCCO2)cn1 ; OCc1cnc(C2CCCCO2)cn1 ; CC1(C)OB(c2cn[nH]c2)OC1(C)C ; CC1(C)OB(c2cnn(Cc3cnc(C4CCCCO4)cn3)c2)OC1(C)C]

Step 1: A mixture of 5-tetrahydropyran-2-ylpyrazine-2-carbonitrile (600 mg, 3.17 mmol, 1 eq) and K₂CO₃ (1.75 g, 12.68 mmol, 4 eq) in H₂O (2 mL) and MeOH (10 mL) was degassed and purged with N₂ for 3 times, and then the mixture was stirred at 25° C. for 12 h under N₂ atmosphere. The aqueous phase was acidified with AcOH until pH=6-7 and stirred at 25° C. for 2 h, and then extracted with ethyl acetate (20 mL×3). The combined organic layers were washed with brine (25 mL), dried over anhydrous Na₂SO₄ and concentrated under reduced pressure to give methyl 5-tetrahydropyran-2-ylpyrazine-2-carboxylate (602 mg, crude) as a white solid. ¹H NMR (400 MHZ, DMSO-d₆) δ 9.13 (d, J=1.2 Hz, 1H), 8.81 (d, J=0.8 Hz, 1H), 4.59 (dd, J=11.2, 2.4 Hz, 1H), 4.06-4.13 (m, 1H), 3.92 (s, 3H), 3.56-3.66 (m, 1H), 1.97-2.04 (m, 1H), 1.86-1.93 (m, 1H), 1.64-1.75 (m, 1H), 1.56-1.62 (m, 2H), 1.45-1.54 (m, 1H). LCMS: calc. for C₁₁H₁₄N₂O₃:222.1, found: [M+H]⁺223.1.

Step 2: To a solution of methyl 5-tetrahydropyran-2-ylpyrazine-2-carboxylate (600 mg, 2.70 mmol, 1 eq) in EtOH (8 mL) was added NaBH₄ (390 mg, 10.31 mmol, 3.82 eq) at 0° C. The mixture was stirred at 25° C. for 4 h under N₂. The mixture was quenched with sat. aq. NH₄Cl (30 mL) at 0° C. The resulting mixture was extracted with ethyl acetate (30 mL×3). The combined organic phases were washed with brine (50 mL), dried over anhydrous sodium sulfate, filtered and concentrated under vacuum to afford the residue. The residue was purified by flash silica gel chromatography to afford (5-tetrahydropyran-2-ylpyrazin-2-yl) methanol (370 mg, 1.90 mmol, 70.56% yield) as a yellow oil. ¹H NMR (400 MHz, DMSO-d₆) δ 8.61 (s, 1H), 8.57 (d, J=1.2 Hz, 1H), 5.52-5.62 (m, 1H), 4.06-4.13 (m, 1H), 4.61-4.62 (m, 2H), 4.47 (dd, J=11.2, 2.4 Hz, 1H), 4.02-4.09 (m, 1H), 3.54-3.62 (m, 1H), 1.86-1.97 (m, 2H), 1.62-1.73 (m, 1H), 1.55-1.62 (m, 2H), 1.46-1.52 (m, 1H). LCMS: calc. for C₁₀H₁₄N₂O₂:194.1, found: [M+H]⁺195.1.

Step 3: To a mixture of (5-tetrahydropyran-2-ylpyrazin-2-yl) methanol (50 mg, 257.43 μmol, 1 eq) and 4-(4,4,5,5-tetramethyl-1,3,2-dioxaborolan-2-yl)-1H-pyrazole (59.94 mg, 308.9 μmol, 1.2 eq) in toluene (1 mL) was added 2-(tributyl-25-phosphanylidene) acetonitrile (310.6 mg, 1.29 mmol, 5 eq). The mixture was stirred at 100° C. for 12 h under N₂ atmosphere. The reaction mixture was poured into water (10 mL) and it was extracted with ethyl acetate (10 mL×3). The combined organic layers were washed with brine (20 mL), dried over anhydrous Na₂SO₄ and concentrated under reduced pressure to give a residue. The residue was purified by prep-TLC to give 2-tetrahydropyran-2-yl-5-[[4-(4,4,5,5-tetramethyl-1,3,2-dioxaborolan-2-yl)pyrazol-1-yl]methyl]pyrazine (67 mg, 115.81 μmol, 44.99% yield, 64% purity) as a brown oil. LCMS: calc. for C₁₉H₂₇BN₄O₃: 370.2, found: [M+H]⁺371.2.

### Synthesis of 5-(cyclopropylmethoxy)-2-[[4-(4,4,5,5-tetramethyl-1,3,2-dioxaborolan-2-yl)pyrazol-1-yl]methyl]pyridine

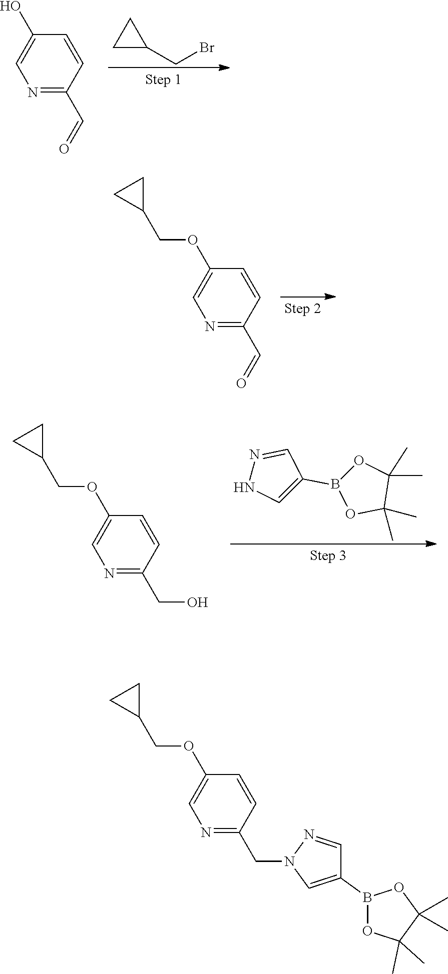

Step 1: To a solution of 5-hydroxypyridine-2-carbaldehyde (2 g, 16.25 mmol, 1 eq), 5-hydroxypyridine-2-carbaldehyde (2 g, 16.25 mmol, 1 eq) in DMF (5 mL) was added bromomethylcyclopropane (2.19 g, 16.25 mmol, 1.56 mL, 1 eq), K₂CO₃ (3.37 g, 24.37 mmol, 1.5 eq). The mixture was stirred at 80° C. for 2 h. The reaction mixture was concentrated. Water (30 mL) was added to the residue. The resulting mixture was extracted with DCM (30 mL×3). The combined organic phase was washed with brine (50 mL), dried over anhydrous Na₂SO₄, filtered and concentrated to give 5-(cyclopropylmethoxy)pyridine-2-carbaldehyde (2 g, 69.47% yield) as a brown oil. LCMS: calc. for C₁₁H₁₃NO₂:177.1, found: [M+H]⁺177.9.

Step 2: To a solution of 5-(cyclopropylmethoxy)pyridine-2-carbaldehyde (2 g, 11.29 mmol, 1 eq) in MeOH (20 mL) was added NaBH₄ (0.89 g, 23.52 mmol, 2.08 eq) at 0° C. The mixture was stirred at 30° C. for 2 h. The reaction mixture was quenched by sat. NH₄Cl (30 mL). The mixture was concentrated with EtOAc (50 ml×3). The organic layers were concentrated to give residue. The residue was purified by flash silica gel chromatography to give [5-(cyclopropylmethoxy)-2-pyridyl]methanol (1.27 g, 62.79% yield) as a yellow oil. LCMS: calc. for C₁₀H₁₃NO₂:179.1, found: [M+H]⁺180.1.

Step 3: To a solution of 4-(4,4,5,5-tetramethyl-1,3,2-dioxaborolan-2-yl)-1H-pyrazole (300 mg, 1.55 mmol, 1 eq), [5-(cyclopropylmethoxy)-2-pyridyl]methanol (277.08 mg, 1.55 mmol, 1 eq) and PPh3 (608.28 mg, 2.32 mmol, 1.5 eq) in THF (6 mL) was added DBAD (534.01 mg, 2.32 mmol, 1.5 eq) at 0° C. The reaction was stirred 16 h at 25° C. under N₂ atmosphere. Water (20 mL) was added to the residue. The resulting mixture was extracted with EtOAc (20 mL×3). The combined organic phase was washed with water (15 mL), dried over anhydrous Na₂SO₄, filtered and concentrated. The residue was purified by flash silica gel chromatography to give 5-(cyclopropylmethoxy)-2-[4-(4,4,5,5-tetramethyl-1,3,2-dioxaborolan-2-yl)pyrazol-1-yl]methyl]pyridine (335 mg, 34.64% yield) as a yellow oil. LCMS: calc. for C₁₉H₂₆BN₃O₃: 355.2, found: [M+H]⁺355.7.

### Synthesis of 4-[6-[[4-(4,4,5,5-tetramethyl-1,3,2-dioxaborolan-2-yl)pyrazol-1-yl]methyl]pyridazin-3-yl]morpholine

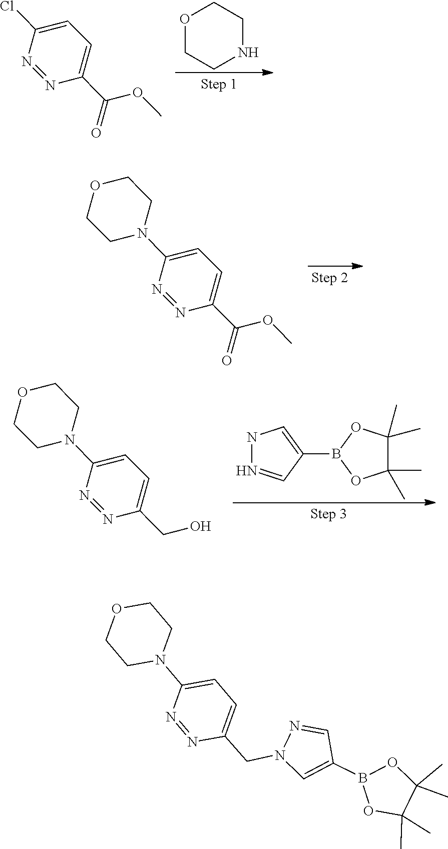 [SMILES: COC(=O)c1ccc(Cl)nn1 ; C1COCCN1 ; COC(=O)c1ccc(N2CCOCC2)nn1 ; OCc1ccc(N2CCOCC2)nn1 ; CC1(C)OB(c2cn[nH]c2)OC1(C)C ; CC1(C)OB(c2cnn(Cc3ccc(N4CCOCC4)nn3)c2)OC1(C)C]

Step 1: A mixture of methyl 6-chloropyridazine-3-carboxylate (2 g, 11.59 mmol, 1 eq), morpholine (1.21 g, 13.91 mmol, 1.22 mL, 1.2 eq), K₂CO₃ (1.92 g, 13.91 mmol, 1.2 eq) in DMSO (20 mL) was degassed and purged with N₂ for 3 times, and then the mixture was stirred at 110° C. for 16 h under N₂ atmosphere. The reaction mixture was added H₂O (50 mL) at 20° C. and extracted with EtOAc (50 mL×3). The combined organic layers were washed with brine (100 mL), dried over Na₂SO₄, filtered and concentrated. The residue was purified by flash silica gel chromatography to give methyl 6-morpholinopyridazine-3-carboxylate (1 g, 4.48 mmol, 38.65% yield) as a yellow solid. ¹HNMR (400 MHZ, DMSO-d₆) δ 7.87 (d, J=9.6 Hz, 1H), 7.29 (d, J=9.6 Hz, 1H), 3.87 (s, 3H), 3.76-3.65 (m, 8H).

Step 2: To a solution of methyl 6-morpholinopyridazine-3-carboxylate (600 mg, 2.69 mmol, 1 eq) in MeOH (8 mL) was added NaBH₄ (260 mg, 6.87 mmol, 2.56 eq) at 0° C. The reaction mixture was stirred at 25° C. for 2 h. The mixture was quenched with NH₄Cl at. (10 mL), extracted with ethyl acetate (30 mL×2). The combined organic extracts were dried over sodium sulphate, filtered and concentrated. The residue was purified by flash silica gel chromatography to give (6-morpholinopyridazin-3-yl) methanol (110 mg, 563.47 μmol, 20.96% yield) as a white solid. ¹HNMR (400 MHZ, DMSO-d₆) ¿ 7.46 (d, J=9.6 Hz, 1H), 7.30 (d, J=9.6 Hz, 1H), 5.38 (t, J=6.0 Hz, 1H), 4.58 (d, J=6.0 Hz, 2H), 3.77-3.68 (m, 4H), 3.55-3.46 (m, 4H).

Step 3: A mixture of (6-morpholinopyridazin-3-yl) methanol (110 mg, 563.47 μmol, 1 eq), 4-(4,4,5,5-tetramethyl-1,3,2-dioxaborolan-2-yl)-1H-pyrazole (87.47 mg, 450.78 μmol, 0.8 eq), 2-(tributyl-phosphanylidene) acetonitrile (339.99 mg, 1.41 mmol, 2.5 eq) in toluene (5 mL) was degassed and purged with N₂ for 3 times, and then the mixture was stirred at 110° C. for 16 h under N₂ atmosphere. The reaction mixture was concentrated directly. The residue was purified by flash silica gel chromatography to give 4-[6-[[4-(4,4,5,5-tetramethyl-1,3,2-dioxaborolan-2-yl)pyrazol-1-yl]methyl]pyridazin-3-yl]morpholine (200 mg, 447.15 μmol, 79.36% yield, 83% purity) as a brown oil.

### Synthesis of 5-(3,3-difluoroazetidin-1-yl)-2-((4-(4,4,5,5-tetramethyl-1,3,2-dioxaborolan-2-yl)-1H-pyrazol-1-yl)methyl)pyrimidine

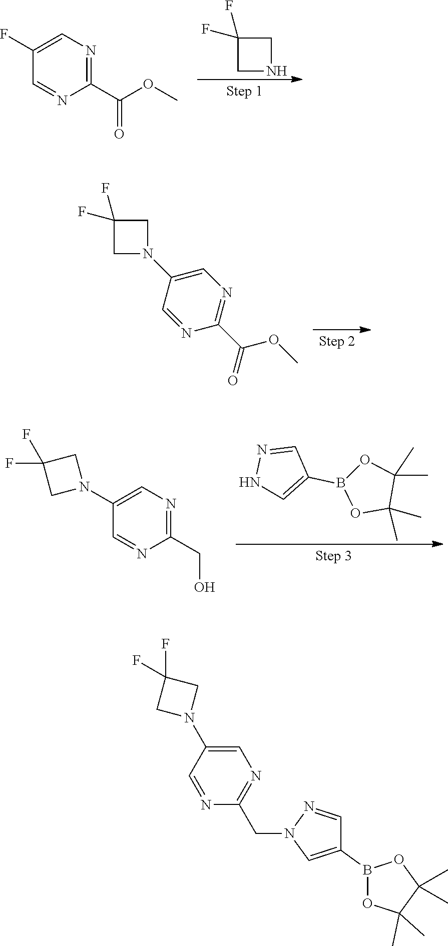 [SMILES: COC(=O)c1ncc(F)cn1 ; FC1(F)CNC1 ; COC(=O)c1ncc(N2CC(F)(F)C2)cn1 ; OCc1ncc(N2CC(F)(F)C2)cn1 ; CC1(C)OB(c2cn[nH]c2)OC1(C)C ; CC1(C)OB(c2cnn(Cc3ncc(N4CC(F)(F)C4)cn3)c2)OC1(C)C]

Step 1: The mixture of methyl 5-fluoropyrimidine-2-carboxylate (2 g, 12.81 mmol, 1 eq), 3,3-difluoroazetidine; hydrochloride (1.66 g, 12.81 mmol, 1 eq), K₂CO₃ (8.85 g, 64.06 mmol, 5 eq) in DMSO (40 mL) was degassed and purged with N₂ for 3 times, and then the mixture was stirred at 100° C. for 16 h under N₂ atmosphere. H₂O (50 mL) was added to the residue. The resulting mixture was extracted with EtOAc (50 mL×3). The combined organic phase was washed with water (100 mL), dried over anhydrous Na₂SO₄, filtered and concentrated. The residue was purified by flash silica gel chromatography to give methyl 5-(3, 3-difluoroazetidin-1-yl)pyrimidine-2-carboxylate (1.63 g, 62.75% yield) as a white solid. LCMS: calc. for C₉H₉F₂N₃O₂:229.1, found: [M+H]⁺230.0.

Step 2: To a solution of methyl 5-(3, 3-difluoroazetidin-1-yl)pyrimidine-2-carboxylate (1.5 g, 6.54 mmol, 1 eq) in MeOH (20 mL) was added NaBH₄ (0.73 g, 19.30 mmol, 2.95 eq). The mixture was stirred at 25° C. for 2 h. The solution was cooled to 0° C. and poured into NH₄Cl (30 mL). The aqueous phase was extracted with EtOAc (30 mL×3). The combined organic phase was washed with brine (50 mL), dried with anhydrous Na₂SO₄, filtered and concentrated in vacuum. The residue was purified by flash silica gel chromatography to give (5-(3, 3-difluoroazetidin-1-yl)pyrimidin-2-yl) methanol (561 mg, 42.61% yield) as a white solid. LCMS: calc. for C₈H₉F₂N₃O: 201.1, found: [M+H]⁺201.9.

Step 3: To a mixture of [5-(3,3-difluoroazetidin-1-yl)pyrimidin-2-yl]methanol (561 mg, 2.79 mmol, 1 eq), 4-(4,4,5,5-tetramethyl-1,3,2-dioxaborolan-2-yl)-1H-pyrazole (486.99 mg, 2.51 mmol, 0.9 eq), 2-(tributyl-phosphanylidene) acetonitrile (1.68 g, 6.97 mmol, 2.5 eq) in toluene (6 mL) was degassed and purged with N₂ for 3 times, and then the mixture was stirred at 110° C. for 12 h under N₂ atmosphere. The residue was poured into H₂O (20 mL). The aqueous phase was extracted with EtOAc (25 mL×3). The combined organic phase was washed with brine (50 mL), dried with anhydrous Na₂SO₄, filtered and concentrated in vacuum. The residue was purified by prep-TLC to give 5-(3,3-difluoroazetidin-1-yl)-2-((4-(4,4,5,5-tetramethyl-1,3,2-dioxaborolan-2-yl)-1H-pyrazol-1-yl)methyl)pyrimidine (1.04 g, 85.96% yield) as a white solid. LCMS: calc. for C₁₇H₂₂BF2N₅O₂: 377.2, found: [M+H]⁺377.9.

### Synthesis of 4-[5-[[4-(4,4,5,5-tetramethyl-1,3,2-dioxaborolan-2-yl)pyrazol-1-yl]methyl]pyrazin-2-yl]morpholine

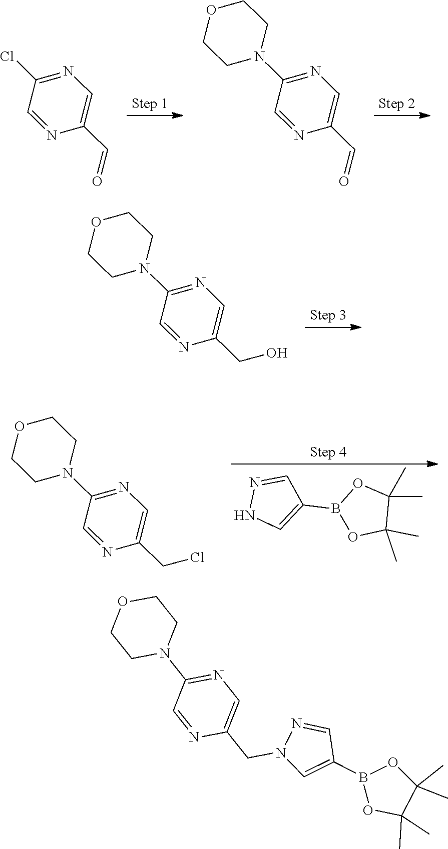

Step 1: To a mixture of 5-chloropyrazine-2-carbaldehyde (2 g, 14.0 mmol, 1 eq), K₂CO₃ (4.85 g, 35.0 mmol, 2.5 eq) and morpholine (1.34 g, 15.4 mmol, 1.1 eq) in ACN(20 mL) was degassed and purged with N₂ for 3 times, and then the mixture was stirred at 80° C. for 1 h under N₂ atmosphere. Water (20 mL) was added to the residue. The resulting mixture was extracted with EtOAc (20 mL×3). The combined organic phase was washed with water, dried over anhydrous Na₂SO₄, filtered and concentrated under reduced pressure to give 5-morpholinopyrazine-2-carbaldehyde (2.5 g, 92.2% yield) as a yellow liquid. ¹HNMR (400 MHz, DMSO-d₆) δ 9.79 (s, 1H), 8.61-8.59 (m, 1H), 8.45 (d, J=1.2 Hz, 1H), 3.78-3.75 (m, 4H), 3.73-3.70 (m, 4H).

Step 2: A solution of 5-morpholinopyrazine-2-carbaldehyde (2.50 g, 12.94 mmol, 1 eq) in MeCN(30 mL) were added to a single-necked flask, then NaBH₄ (1.5 g, 39.65 mmol, 3.06 eq) was added at 0° C., and the reaction was stirred at 25° C. for 1 h. The reaction was quenched with H₂O (40 mL) drop-wise. The resulting mixture was extracted with EtOAc (40 mL×3). The combined organic phase was washed with brine (50 mL), dried over anhydrous Na₂SO₄, filtered and concentrated. The residue was purified by flash silica gel chromatography to give (5-morpholinopyrazin-2-yl) methanol (2.31 g, 91.45% yield) as a yellow solid. ¹HNMR (400 MHZ, DMSO-d₆) δ 8.23 (s, 1H), 8.13 (s, 1H), 5.23 (t, J=5.6 Hz, 1H), 4.46 (d, J=5.6 Hz., 2H), 3.72-3.69 (m, 4H), 3.50-3.47 (m, 4H).

Step 3: To a solution of (5-morpholinopyrazin-2-yl) methanol (300 mg, 1.54 mmol, 1 eq) in DCM (2 mL) was added thionyl chloride (639.89 mg, 5.38 mmol, 3.5 eq) at 0° C. The mixture was stirred at 25° C. for 1 h. The reaction mixture was concentrated under reduced pressure to give 4-[5-(chloromethyl)pyrazin-2-yl]morpholine (390 mg, crude) as a light yellow liquid. HNMR (400 MHZ, DMSO-d₆) δ 8.31 (d, J=1.2 Hz, 1H), 8.22 (d, J=1.2 Hz, 1H), 4.73 (s, 2H), 3.71-3.68 (m, 4H), 3.57-3.54 (m, 4H).

Step 4: To a solution of 4-[5-(chloromethyl)pyrazin-2-yl]morpholine (280 mg, 1.31 mmol, 1 eq) and 4-(4,4,5,5-tetramethyl-1,3,2-dioxaborolan-2-yl)-1H-pyrazole (254.28 mg, 1.31 mmol, 1 eq) in ACN(5 mL) was added Cs₂CO₃ (1.07 g, 3.28 mmol, 2.5 eq). The mixture was stirred at 80° C. for 1 h. The reaction mixture was concentrated. The residue was diluted with H₂O (30 mL) and extracted with DCM (20 mL×3). The combined organic layers were washed with brine (20 mL), dried over anhydrous Na₂SO₄, concentrated to give 4-[5-[[4-(4,4,5,5-tetramethyl-1,3,2-dioxaborolan-2-yl)pyrazol-1-yl]methyl]pyrazin-2-yl]morpholine (400 mg, 52.62% yield) as a white solid. HNMR (400 MHZ, DMSO-d₆) δ 8.25 (d, J=1.2 Hz, 1H), 8.08 (d, J=1.2 Hz., 1H), 7.97 (s, 1H), 7.56 (s, 1H), 5.77-5.74 (m, 2H), 3.70-3.67 (m, 4H), 3.51-3.49 (m, 4H), 1.24 (s, 12H).

### Synthesis of N, N-dimethyl-2-[2-[[4-(4,4,5,5-tetramethyl-1,3,2-dioxaborolan-2-yl)pyrazol-1-yl]methyl]pyrimidin-5-yl]oxy-ethanamine

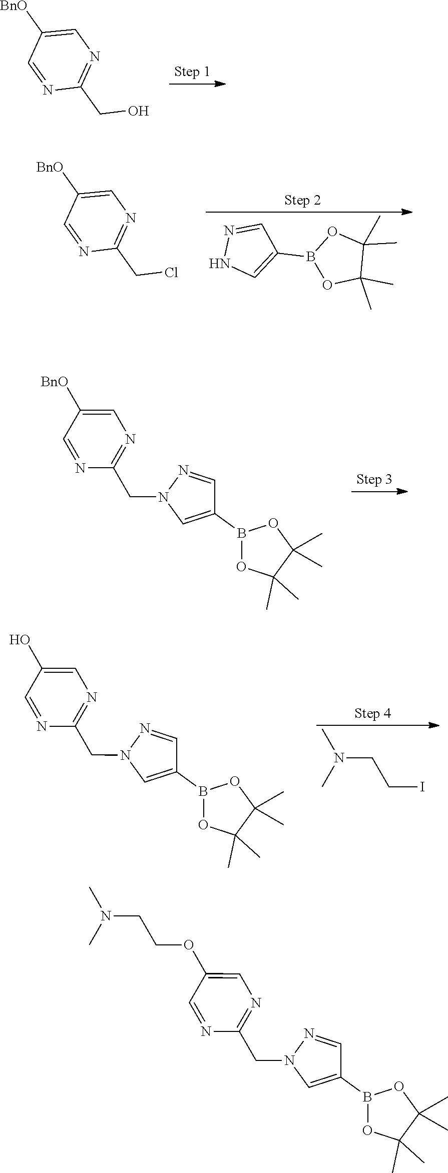

Step 1: To a solution of (5-benzyloxypyrimidin-2-yl) methanol (150 mg, 693.69 μmol, 1 eq) in DCM (4 mL) was added SOCl₂ (247.58 mg, 2.08 mmol, 3 eq) dropwise at 0° C., purged with N₂ for 3 times, and then the mixture was stirred at 25° C. for 0.5 h under N₂ atmosphere. The reaction mixture was concentrated to give 5-benzyloxy-2-(chloromethyl)pyrimidine (242 mg, 74.33% yield) as a white solid. ¹H NMR (400 MHZ, DMSO-d₆) δ 8.64 (s, 2H), 7.51-7.46 (m, 2H), 7.42 (t, J=7.2 Hz, 2H), 7.39-7.34 (m, 1H), 5.30 (s, 2H), 4.76 (s, 2H).

Step 2: A solution of 5-benzyloxy-2-(chloromethyl)pyrimidine (190 mg, 809.61 μmol, 1 eq), 4-(4,4,5,5-tetramethyl-1,3,2-dioxaborolan-2-yl)-1H-pyrazole (157.09 mg, 809.61 μmol, 1 eq), Cs₂CO₃ (791.36 mg, 2.43 mmol, 3 eq) in ACN(10 mL) was degassed and purged with N₂ for 3 times, and then the mixture was stirred at 80° C. for 1.5 h under N₂ atmosphere. The reaction mixture was concentrated. The residue was diluted with H₂O (30 mL) and extracted with DCM (20 mL×3). The combined organic layers were washed with brine (20 mL), dried over anhydrous Na₂SO₄, filtered and concentrated to give 5-benzyloxy-2-[[4-(4,4,5,5-tetramethyl-1,3,2-dioxaborolan-2-yl)pyrazol-1-yl]methyl]pyrimidine (350 mg, crude) as a yellow oil.

Step 3: To a solution of 5-benzyloxy-2-[[4-(4,4,5,5-tetramethyl-1,3,2-dioxaborolan-2-yl)pyrazol-1-yl]methyl]pyrimidine (350 mg, 892.27 μmol, 1 eq) in MeOH (10 mL) was added Pd/C (3.50 g, 3.29 mmol, 10% purity, 3.69 eq) under N₂ atmosphere. The suspension was degassed and purged with H₂ for 3 times. The mixture was stirred under H₂ (15 Psi) at 25° C. for 1 h. The reaction mixture was filtered. The filtrate was concentrated to give 2-[4-(4,4,5,5-tetramethyl-1,3,2-dioxaborolan-2-yl)pyrazol-1-yl]methyl]pyrimidin-5-ol (220 mg, 81.61% yield) as a white solid. ¹HNMR (400 MHZ, DMSO-d₆) & 10.63 (br s, 1H), 8.27 (s, 2H), 7.99 (s, 1H), 7.55 (s, 1H), 5.42 (s, 2H), 1.27-1.23 (m, 12H).

Step 4: To a solution of 2-[4-(4,4,5,5-tetramethyl-1,3,2-dioxaborolan-2-yl)pyrazol-1-yl]methyl]pyrimidin-5-ol (60 mg, 198.59 μmol, 1 eq) and 2-iodo-N,N-dimethyl-ethanamine: hydroiodide (64.93 mg, 198.59 μmol, 1 eq) in ACN(2 mL) was added Cs₂CO₃ (194.11 mg, 595.76 μmol, 3 eq). The mixture was stirred at 80° C. for 2 h. The reaction mixture was filtered. The filtrate was concentrated to give N,N-dimethyl-2-[2-[4-(4,4,5,5-tetramethyl-1,3,2-dioxaborolan-2-yl)pyrazol-1-yl]methyl]pyrimidin-5-yl]oxy-ethanamine (80 mg, crude) as a yellow oil.

### Synthesis of N-(2-methoxyethyl)-N-methyl-2-[[4-(4,4,5,5-tetramethyl-1,3,2-dioxaborolan-2-yl)pyrazol-1-yl]methyl]pyrimidin-5-amine

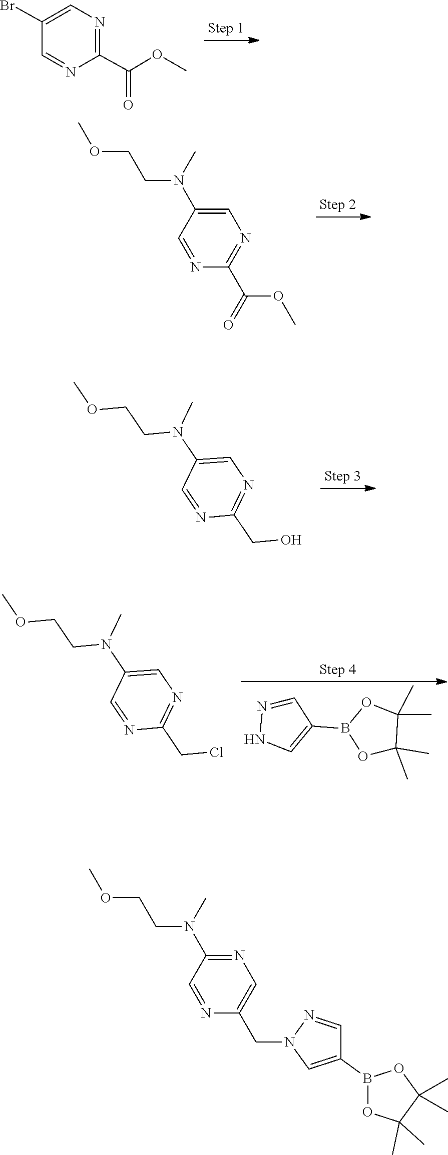

Step 1: A mixture of methyl 5-bromopyrimidine-2-carboxylate (1.87 g, 8.62 mmol, 1 eq), 2-methoxy-N-methyl-ethanamine (3.07 g, 34.47 mmol, 4 eq), (5-diphenylphosphanyl-9,9-dimethyl-xanthen-4-yl)-diphenyl-phosphane (997.16 mg, 1.72 mmol, 0.2 eq), Pd(OAc)_(193.45 mg, 861.67 μmol, 0.1 eq) and Cs₂CO₃ (14.04 g, 43.08 mmol, 5 eq) in dioxane (15 mL) was purged with N₂ for 3 times, and then the mixture was stirred at 100° C. for 1.5 h under N₂ atmosphere. The reaction mixture was filtered and the filtrate and concentrate. The residue was purified by flash silica gel chromatography to give methyl 5-[2-methoxyethyl(methyl)amino]pyrimidine-2-carboxylate (1.02 g, 49.78% yield) as a yellow solid. ¹H NMR (400 MHZ, DMSO-d₆) δ 8.35 (s, 2H), 3.81 (s, 3H), 3.69-3.63 (m, 2H), 3.56-3.51 (m, 2H), 3.26-3.21 (m, 3H), 3.05 (s, 3H). LCMS: calc. for C₁₀H₁₅N₃O₃:225.1, found: [M+H]⁺226.0.

Step 2: A solution of methyl 5-[2-methoxyethyl(methyl)amino]pyrimidine-2-carboxylate (510 mg, 2.26 mmol, 1 eq) in THF (20 mL) was purged with N₂ for 3 times, then LiAlH₄ (2.5 M, 2.72 mL, 3 eq) was added under N₂ atmosphere. The mixture was stirred at 0° C. for 2 h, followed by addition of H₂O (258 μL), 15% NaOH (258 μL) and H₂O (774 L). The mixture was extracted with EtOAc. The organic phase was dried over Na₂SO₄, filtered and concentrated under reduced pressure to give [5-[2-methoxyethyl(methyl)amino]pyrimidin-2-yl]methanol (390 mg, 80.66% yield) as a brown oil. ¹H NMR (400 MHZ, CDCl₃) δ 8.23 (s, 2H), 4.76-4.72 (m, 2H), 3.56 (dd, J=4.4, 8.4 Hz, 4H), 3.36 (s, 3H₁), 3.03 (s, 3H). LCMS: calc. for C₉H15N₃O₂:197.1, found: [M+H]⁺197.9.

Step 3: To a mixture of [5-[2-methoxyethyl(methyl)amino]pyrimidin-2-yl]methanol (190 mg, 963.32 μmol, 1 eq) in DCM (5 mL) was added SOCl₂ (401.12 mg, 3.37 mmol, 3.5 eq) at 0° C. The mixture was stirred at 30° C. for 1 h. The reaction mixture was concentrated to give 2-(chloromethyl)-N-(2-methoxyethyl)-N-methyl-pyrimidin-5-amine (220 mg, crude) as a dark brown solid. ¹H NMR (400 MHz, CDCl₃) δ 8.38-8.29 (m, 2H), 7.82-7.81 (m, 1H), 4.87-4.79 (m, 2H), 3.62-3.58 (m, 4H), 3.36-3.32 (m, 3H), 3.10 (s, 3H).

Step 4: To a solution of 2-(chloromethyl)-N-(2-methoxyethyl)-N-methyl-pyrimidin-5-amine (220 mg, 816.03 μmol, 1 eq) in ACN(5 mL) was added 4-(4,4,5,5-tetramethyl-1,3,2-dioxaborolan-2-yl)-1H-pyrazole (142.51 mg, 734.42 μmol, 0.9 eq) and K₂CO₃ (338.35 mg, 2.45 mmol, 3 eq). The mixture was stirred at 85° C. for 16 h. The reaction mixture was filtered, and the filtrate was concentrated. The residue was purified by flash silica gel chromatography to give N-(2-methoxyethyl)-N-methyl-2-[[4-(4,4,5,5-tetramethyl-1,3,2-dioxaborolan-2-yl)pyrazol-1-yl]methyl]pyrimidin-5-amine (67 mg, 7.89% yield) as a yellow oil. LCMS: calc. for C₁₈H₂₈BN₅O₃: 373.2, found: [M+H]⁺374.1.

### Synthesis of 4-[5-[[4-(4,4,5,5-tetramethyl-1,3,2-dioxaborolan-2-yl)pyrazol-1-yl]methyl]thiazol-2-yl]morpholine

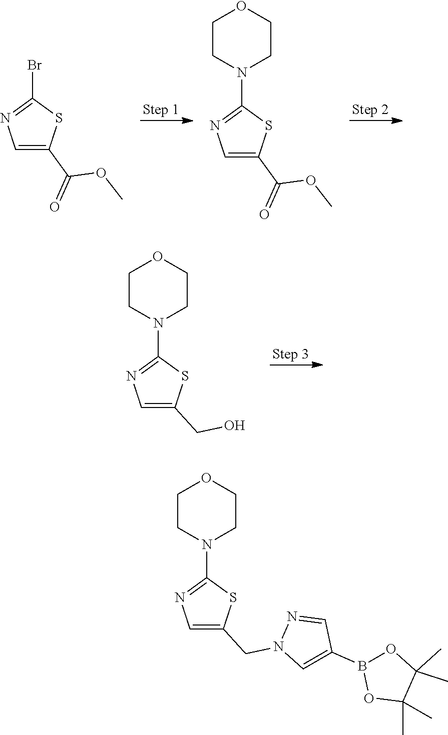

Step 1: A mixture of methyl 2-bromothiazole-5-carboxylate (5 g, 22.52 mmol, 1 eq), morpholine (2.0 g, 22.52 mmol, 1 eq), Cs₂CO₃ (18.3 g, 56.29 mmol, 2.5 eq) in ACN(50 mL) was purged with N₂ for 3 times, and then the mixture was stirred at 80° C. for 2 h under N₂ atmosphere. The reaction mixture was concentrated. aq. NH₄Cl (50 mL) was added to the residue. The resulting mixture was extracted with EtOAc (50 mL×3). The combined organic phase was washed with brine (100 mL), dried over anhydrous Na₂SO₄, filtered and concentrated. The residue was purified by flash silica gel chromatography to give methyl 2-morpholinothiazole-5-carboxylate (2.84 g, 43.27% yield) as a light yellow solid. ¹H NMR (400 MHZ, CDCl₃) δ 7.89 (s, 1H), 3.84 (s, 3H), 3.84-3.81 (m, 4H), 3.58-3.54 (m, 4H). LCMS: calc. for C₉H12N₂O₃S: 228.0, found: [M+H]⁺228.9.

Step 2: A mixture of methyl 2-morpholinothiazole-5-carboxylate (1 g, 4.38 mmol, 1 eq) in THF (15 mL) was purged with N₂ for 3 times, and then LAH (498.8 mg, 13.14 mmol, 3 e) was added dropwise at 0° C. under N₂ atmosphere. The mixture was stirred at 25° C. for 1 h under N₂ atmosphere. To the mixture stirred at 0° C. was added H₂O (5.26 mL), then 15% NaOH (5.26 mL) solution, and then H₂O (16 mL). The mixture was extracted with EtOAc. The organic phase was dried over Na₂SO₄, filtered and concentrated under reduced pressure to give a residue. The residue was purified by flash silica gel chromatography to give 2-morpholinothiazol-5-yl) methanol (460 mg, 52.43% yield) as a light yellow oil. ¹H NMR (400 MHz, DMSO-d₆) § 6.94 (s, 1H), 4.51-4.49 (m, 2H), 4.17-4.16 (m, 1H), 3.71-3.67 (m, 4H), 3.32-3.29 (m, 4H).

Step 3: To a solution of (2-morpholinothiazol-5-yl) methanol (200 mg, 998.71 μmol, 1 eq) and 4-(4,4,5,5-tetramethyl-1,3,2-dioxaborolan-2-yl)-1H-pyrazole (193.8 mg, 998.71 μmol, 1 eq) in toluene (4 mL) was added TBUP (303.1 mg, 1.50 mmol, 1.5 eq) and TMAD (257.9 mg, 1.50 mmol, 1.5 eq). The mixture was stirred at 25° C. for 16 h. The reaction mixture was concentrated. H₂O (10 mL) was added to the residue. The resulting mixture was extracted with EtOAc (10 mL×3). The combined organic phase was washed with brine (20 mL), dried over anhydrous Na₂SO₄, filtered and concentrated. The residue was purified by flash silica gel chromatography to give 4-[5-[[4-(4,4,5,5-tetramethyl-1,3,2-dioxaborolan-2-yl)pyrazol-1-yl]methyl]thiazol-2-yl]morpholine (160 mg, 37.44% yield) as a colourless oil. ¹H NMR (400 MHZ, CDCl₃) δ 7.81 (s, 1H), 7.71 (s, 1H), 7.19 (s, 1H), 5.34 (s, 2H), 3.82-3.79 (m, 4H), 3.51-3.45 (m, 4H), 1.31 (s, 12H). LCMS: calc. for C₁₇H₂₅BN₄O₃S: 376.2, found: [M+H]⁺179.0.

### Section II: Synthesis of the Compounds of the Application

### Examples 1 and 2: Synthesis of tert-butyl 2-(3-(2,6-dioxopiperidin-3-yl)benzofuran-6-yl)-1H-indole-1-carboxylate (Example 1) and 3-(6-(1H-indol-2-yl)benzofuran-3-yl)piperidine-2,6-dione (Example 2)

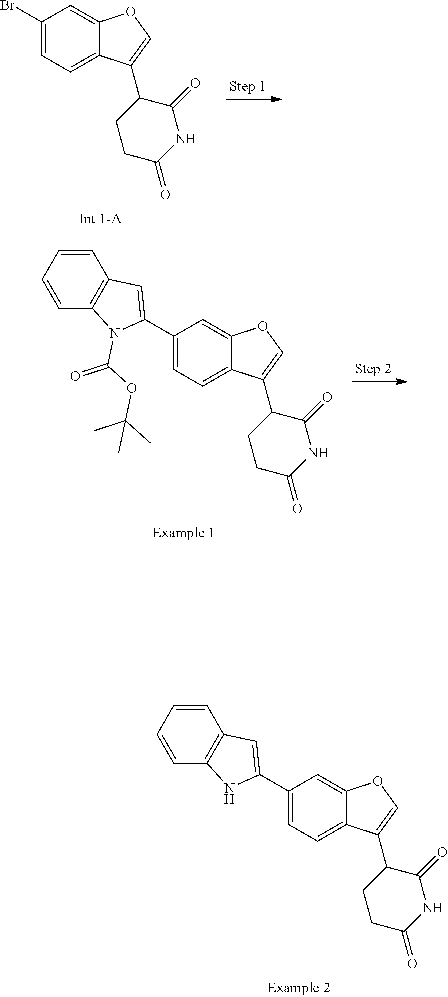 [SMILES: O=C1CCC(c2coc3cc(Br)ccc23)C(=O)N1 ; CC(C)(C)OC(=O)n1c(-c2ccc3c(C4CCC(=O)NC4=O)coc3c2)cc2ccccc21 ; O=C1CCC(c2coc3cc(-c4cc5ccccc5[nH]4)ccc23)C(=O)N1]

Step 1: To a solution of Int 1-A (100 mg, 324.54 μmol, 1 eq), (1-tert-butoxycarbonylindol-2-yl) boronic acid (101.68 mg, 389.45 μmol, 1.2 eq) and Na₂CO₃ (103.19 mg, 973.62 umol, 3 eq) in dioxane (5 mL) and H₂O (0.5 mL) was added Pd(dppf)Cl₂ (11.87 mg, 16.23 umol, 0.05 eq). The mixture was stirred at 100° C. for 6 hr under N₂. The reaction mixture was separated and the aqueous layer was extracted with EtOAc. The combined organic layers was dried with anhydrous Na₂SO₄, filtered and concentrated under reduced pressure to give crude product. The residue was purified by prep-HPLC to afford the Example 1 (24.3 mg, 53.6 umol, 16.5% yield, 98% purity) as a white solid. LCMS: calc. for C₂₆H₂₄N₂O₅:444.2, found: [M−Boc+H]⁺345.2 ¹H NMR (400 MHZ, DMSO-d₆) & 10.92 (s, 1H), 8.09 (d, J=8.4 Hz, 1H), 7.97 (s, 1H), 7.60-7.70 (m, 3H), 7.24-7.37 (m, 3H), 6.77 (s, 1H), 4.19 (dd, J=12.0, 4.8 Hz, 1H), 2.71-2.84 (m, 1H), 2.56-2.64 (m, 1H), 2.31-2.43 (m, 1H), 2.10-2.21 (m, 1H), 1.25 (s, 9H).

Step 2: To a solution of Example 1 (10 mg, 22.50 umol, 1 eq) in HCl/EtOAc (2 mL) stirred at 40° C. for 16 hr. The reaction mixture was concentrated under reduced pressure to give crude product. The residue was purified by prep-TLC to afford the Example 2 (3.8 mg, 9.82 umol, 43.65% yield, 89% purity) as a brown solid. LCMS: calc. for C₂₁H₁₆N₂O₃: 344.2, found: [M+H]⁺345.2 ¹H NMR (400 MHZ, DMSO-d₆) δ 11.57 (s, 1H), 10.93 (s, 1H), 8.08 (s, 1H), 7.95 (s, 1H), 7.77-7.81 (m, 1H), 7.65-7.67 (m, 1H), 7.53 (d, J=7.6 Hz, 1H), 7.41 (d, J=8.0 Hz, 1H), 7.10 (t, J=7.6 Hz, 1H), 6.96-7.03 (m, 2H), 4.17 (dd, J=12.0, 4.8 Hz, 1H), 2.72-2.81 (m, 1H), 2.70-2.65 (m, 1H), 2.31-2.40 (m, 1H), 2.12-2.21 (m, 1H).

### Example 3: 3-(6-(1-(4-methoxybenzyl)-1H-pyrazol-4-yl)benzofuran-3-yl)piperidine-2,6-dione

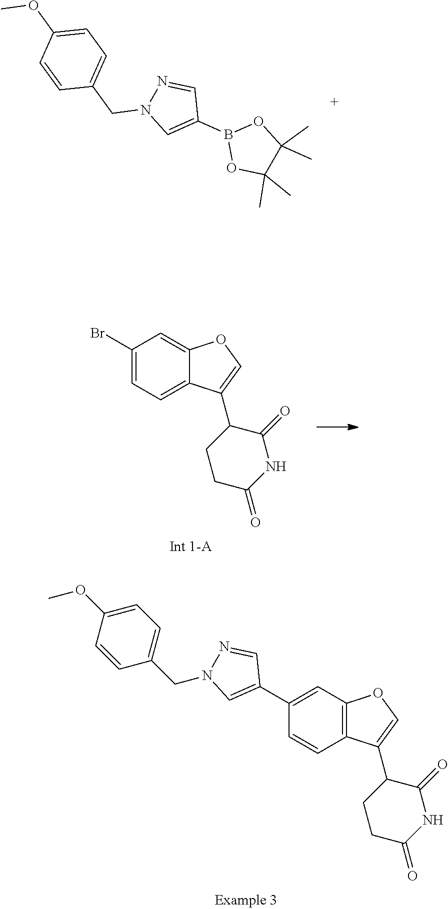

To a solution of Int 1-A and 1-[(4-methoxyphenyl)methyl]-4-(4,4,5,5-tetramethyl-1,3,2-dioxaborolan-2-yl)pyrazole (122.36 mg, 389.45 umol, 1.2 eq) in dioxane (3 mL) and H₂O (0.3 mL) was added in KF (37.71 mg, 649.08 umol, 15.21 uL, 2 eq) and Pd(dppf)Cl₂ (11.87 mg, 16.23 umol, 0.05 eq). The mixture was bubbled by N₂ for 10 min. The mixture was stirred at 85° C. for 16 hr. The reaction mixture was concentrated under reduced pressure. The residue was purified by prep-HPLC to afford the title compound Example 3 (16.0 mg, 38.14 umol, 11.75% yield, 99.04% purity) as a white solid. LCMS: calc. for C₂₄H₂₁N₃O₄:415.2, found: [M+H]⁺416.1. ¹H NMR (400 MHZ, DMSO-d₆) δ 10.90 (s, 1H), 8.29 (s, 1H), 7.96 (s, 1H), 7.85 (s, 1H), 7.78 (s, 1H), 7.52-7.56 (m, 1H), 7.43-7.49 (m, 1H), 7.26 (d, J=8.4 Hz, 2H), 6.91 (d, J=8.8 Hz, 2H), 5.26 (s, 2H), 4.12 (dd, J=12.0, 4.8 Hz, 1H), 3.73 (s, 3H), 2.68-2.80 (m, 1H), 2.53-2.62 (m, 1H), 2.26-2.38 (m, 1H), 2.06-2.18 (m, 1H).

### Example 4: 3-[6-[1-(2,3-dihydrobenzofuran-5-ylmethyl)pyrazol-4-yl]benzofuran-3-yl]piperidine-2,6-dione

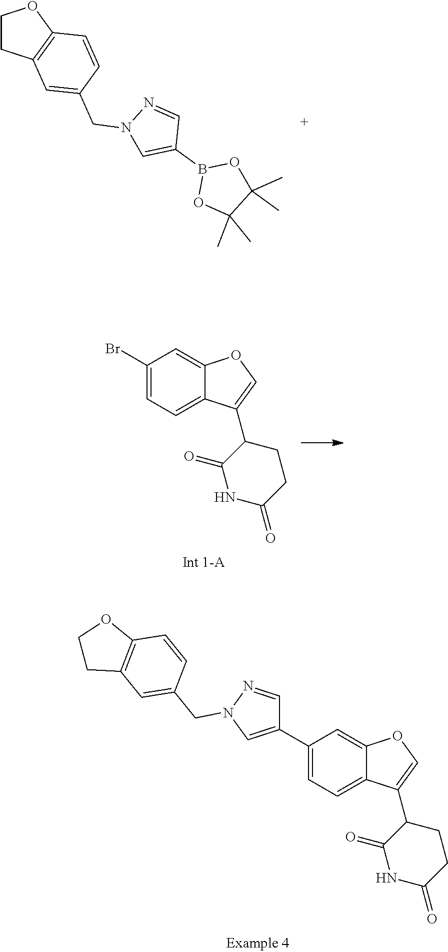 [SMILES: CC1(C)OB(c2cnn(Cc3ccc4c(c3)CCO4)c2)OC1(C)C ; O=C1CCC(c2coc3cc(Br)ccc23)C(=O)N1 ; O=C1CCC(c2coc3cc(-c4cnn(Cc5ccc6c(c5)CCO6)c4)ccc23)C(=O)N1]

A mixture of 1-(2,3-dihydrobenzofuran-5-ylmethyl)-4-(4,4,5,5-tetramethyl-1,3,2-dioxaborolan-2-yl)pyrazole (52.93 mg, 97.36 umol, 60% purity, 1 eq), Int 1-A (30 mg, 97.36 umol, 1 eq), Pd(dppf) C₁₂ (7.12 mg, 9.74 umol, 0.1 eq) and KF (11.31 mg, 194.72 umol, 2 eq) in dioxane (1 mL) and H₂O (0.1 mL) was degassed and purged with N₂ for 3 times, and then the mixture was stirred at 85° C. for 16 hours under N₂ atmosphere. The mixture was filtered directly, and the filtrate was concentrated under reduced pressure to give a residue. The residue was purified by prep-HPLC to afford Example 4 (8.2 mg, 17.96 umol, 18.4% yield) as a white solid. LCMS: calc. for C₂₅H₂₁N₃O₄:427.2, found: [M+H]⁺428.1. ¹H NMR (400 MHz, DMSO-d₆) δ 10.89 (s, 1H), 8.29 (s, 1H), 7.96 (s, 1H), 7.86 (s, 1H), 7.79 (s, 1H), 7.52-7.58 (m, 1H), 7.44-7.51 (m, 1H), 7.21 (s, 1H), 7.09 (d, J=8.0 Hz, 1H), 6.73 (d, J=8.0 Hz, 1H), 5.23 (s, 2H), 4.50 (t, J=8.0 Hz, 2H), 4.09-4.17 (m, 1H), 3.15 (t, J=8.0 Hz, 2H), 2.70-2.79 (m, 1H), 2.51-2.59 (m, 1H), 2.22-2.37 (m, 1H), 2.08-2.18 (m, 1H).

### Example 5: 3-[6-[1-[(2-methoxy-4-pyridyl)methyl]pyrazol-4-yl]benzofuran-3-yl]piperidine-2,6-dione

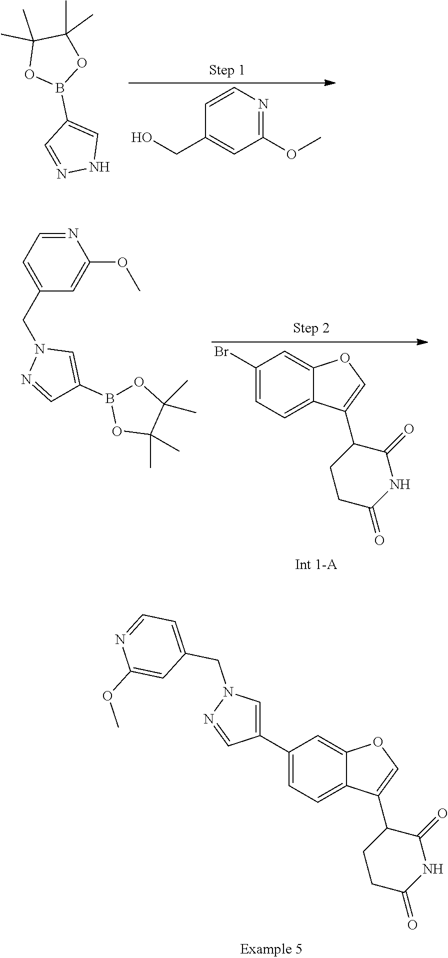

Step 1: A solution of 4-(4,4,5,5-tetramethyl-1,3,2-dioxaborolan-2-yl)-1H-pyrazole (697.22 mg, 3.59 mmol, 1 eq), (2-methoxy-4-pyridyl) methanol (500 mg, 3.59 mmol, 1 eq) and PPh3 (942.46 mg, 3.59 mmol, 1 eq) in THF (10 mL) was added DIAD (726.58 mg, 3.59 mmol, 698.64 μL, 1 eq) at 0° C., and then the mixture was stirred at 20° C. for 16 hours. The reaction mixture was concentrated under reduced pressure to afford a residue. The residue was purified by column chromatography to afford 2-methoxy-4-[[4-(4,4,5,5-tetramethyl-1,3,2-dioxaborolan-2-yl)pyrazol-1-yl]methyl]pyridine (2.04 g, crude) as a yellow oil. LCMS: calc. for C₁₆H₂₂BN₃O₃: 315.2, found: [M+H]⁺315.9. ¹H NMR (400 MHZ, DMSO-d₆) δ 8.88 (s, 1H), 8.06-8.13 (m, 1H), 7.65 (s, 1H), 6.71-6.77 (m, 1H), 6.49 (s, 1H), 5.37 (s, 2H), 3.82 (d, J=6.0 Hz, 3H), 1.18 (d, J=6.0 Hz, 12H).

Step 2: A mixture of Int 1-A (40 mg, 129.82 umol, 1 eq), 2-methoxy-4-[[4-(4,4,5,5-tetramethyl-1,3,2-dioxaborolan-2-yl)pyrazol-1-yl]methyl]pyridine (40.92 mg, 129.82 μmol, 1 eq), K₃PO₄ (55.11 mg, 259.64 μmol, 2 eq) and XPhos Pd G3 (10.99 mg, 12.98 μmol, 0.1 eq) in THF (2 mL) and H₂O (0.2 mL) was degassed and purged with N₂ for 3 times. The mixture was stirred at 60° C. for 2 hours under N₂ atmosphere. The mixture was filtered directly, and the cake was washed with DCM (10 mL). The filtrate was concentrated under reduced pressure to afford a residue. The residue was purified by prep-HPLC to afford Example 5 (1.7 mg, 3.97 μmol, 3.1% yield) as a white solid. LCMS: calc. for C₂₃H₂ON₄O₄:416.2, found: [M+H]⁺417.3. ¹H NMR (400 MHZ, DMSO-d₆) δ 10.90 (s, 1H), 8.37 (s, 1H), 8.12 (d, J=5.6 Hz, 1H), 8.04 (s, 1H), 7.86 (s, 1H), 7.81 (d, J=0.8 Hz, 1H), 7.54-7.58 (m, 1H), 7.47-7.52 (m, 1H), 6.81 (dd, J=5.6, 1.2 Hz, 1H), 6.57 (s, 1H), 5.37 (s, 2H), 4.11-4.15 (m, 1H), 3.82 (s, 3H), 2.70-2.78 (m, 1H), 2.55-2.61 (m, 1H), 2.29-2.36 (m, 1H), 2.09-2.17 (m, 1H).

### Example 6: 3-[6-[1-[(5-methoxy-2-pyridyl)methyl]pyrazol-4-yl]benzofuran-3-yl]piperidine-2,6-dione

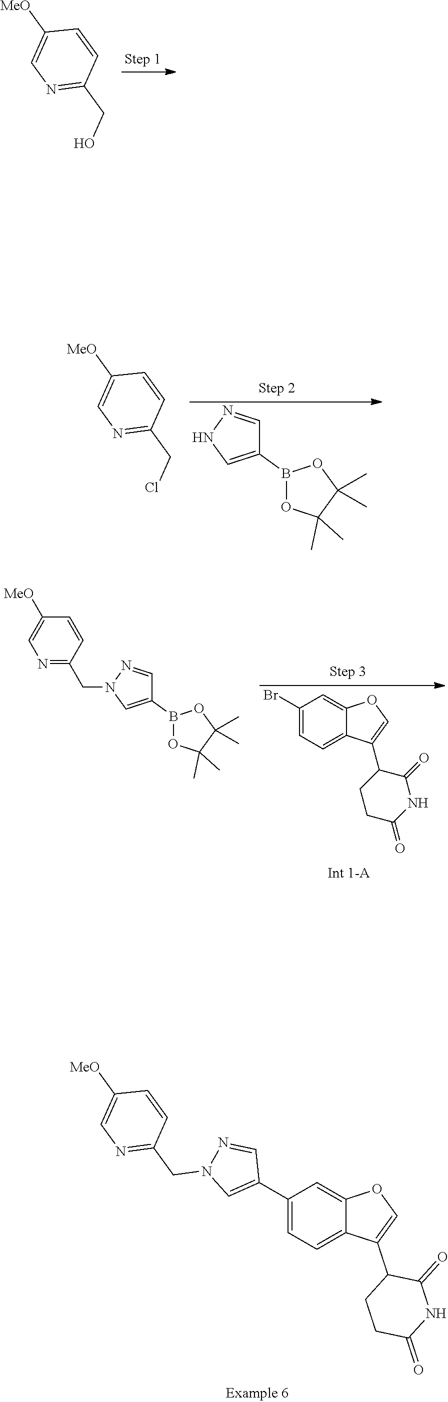

Step 1: A solution of (5-methoxy-2-pyridyl) methanol (1 g, 7.19 mmol, 1 eq) in thionyl chloride (20 mL) was stirred at 80° C. for 1 h. The reaction mixture was concentrated under reduced pressure to afford 2-(chloromethyl)-5-methoxy-pyridine (2.00 g, crude) as a yellow solid. LCMS: calc. for C₇H₈ClNO: 157.0, found: [M+H]⁺158.3.

Step 2: To a solution of 2-(chloromethyl)-5-methoxy-pyridine (1.00 g, 6.35 mmol, 1 eq) and 4-(4,4,5,5-tetramethyl-1,3,2-dioxaborolan-2-yl)-1H-pyrazole (1.23 g, 6.35 mmol, 1 eq) in DMF (10 mL) was added K₂CO₃ (2.63 g, 19.04 mmol, 3 eq). The mixture was stirred at 60° C. for 16 h. The reaction mixture was poured into water (100 mL) and extracted with EtOAc (50 mL×4). The combined organic layer was washed with brine (50 mL), dried over anhydrous Na₂SO₄ and concentrated under reduced pressure to afford a residue. The residue was purified by column chromatography to afford 5-methoxy-2-[[4-(4,4,5,5-tetramethyl-1,3,2-dioxaborolan-2-yl)pyrazol-1-yl]methyl]pyridine (656 mg, 1.81 mmol, 28.54% yield) as a yellow oil. LCMS: calc. for C₁₆H₂₂BN₃O₃: 315.2, found: [M+H]⁺315.7. ¹H NMR (400 MHz, DMSO-d₆): δ 8.23 (d, J=2.8 Hz, 1H), 8.00 (s, 1H), 7.59 (s, 1H), 7.36 (dd, J=8.4, 2.8 Hz, 1H), 7.11 (d, J=8.4 Hz, 1H), 5.36 (s, 2H), 3.80 (s, 3H), 1.24 (s, 12H).

Step 3: A mixture of 5-methoxy-2-[[4-(4,4,5,5-tetramethyl-1,3,2-dioxaborolan-2-yl)pyrazol-1-yl]methyl]pyridine (30.69 mg, 97.36 μmol, 1 eq), Int 1-A (30 mg, 97.36 μmol, 1 eq), Xphos Pd G3 (8.24 mg, 9.74 μmol, 0.1 eq), K₃PO₄ (41.33 mg, 194.72 μmol, 2 eq) in THF (2 mL) and H₂O (0.2 mL) was degassed and purged with N₂ for 3 times, and then the mixture was stirred at 60° C. for 3 hours under N₂ atmosphere. The combined liquid was filtered directly, and the cake was washed with DCM (10 mL). The filtrate was concentrated under reduced pressure to afford a residue. The residue was purified by prep-HPLC to afford Example 6 (4.0 mg, 9.08 μmol, 9.3% yield) as a white solid. LCMS: calc. for C₂₃H₂ON₄O₄:416.1, found: [M+H]⁺417.0. ¹H NMR (400 MHZ, DMSO-d₆) § 10.90 (s, 1H), 8.32 (s, 1H), 8.25 (d, J=2.4 Hz, 1H), 7.98 (s, 1H), 7.85 (s, 1H), 7.80 (d, J=0.8 Hz, 1H), 7.53-7.56 (m, 1H), 7.47-7.50 (m, 1H), 7.39 (dd, J=8.8, 3.2 Hz, 1H), 7.18 (d, J=8.8 Hz, 1H), 5.37 (s, 2H), 4.13 (dd, J=12.0, 4.8 Hz, 1H), 3.81 (s, 3H), 2.61-2.69 (m, 1H), 2.58-2.61 (m, 1H), 2.30-2.35 (m, 1H), 2.12-2.16 (m, 1H).

### Example 7:3-[6-[1-[(5-methoxypyrimidin-2-yl)methyl]pyrazol-4-yl]benzofuran-3-yl]piperidine-2,6-dione

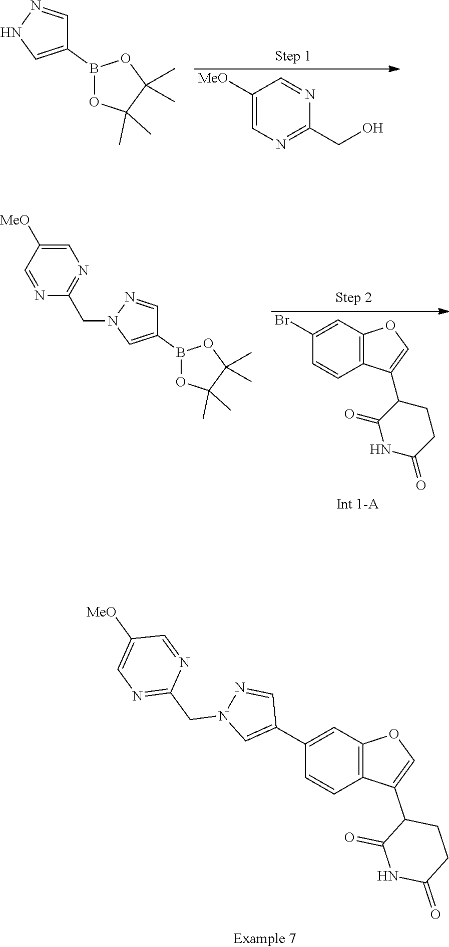 [SMILES: CC1(C)OB(c2cn[nH]c2)OC1(C)C ; COc1cnc(CO)nc1 ; COc1cnc(Cn2cc(B3OC(C)(C)C(C)(C)O3)cn2)nc1 ; O=C1CCC(c2coc3cc(Br)ccc23)C(=O)N1 ; COc1cnc(Cn2cc(-c3ccc4c(C5CCC(=O)NC5=O)coc4c3)cn2)nc1]

Step 1: To a solution of (5-methoxypyrimidin-2-yl) methanol (200 mg, 1.43 mmol, 1 eq), 4-(4,4,5,5-tetramethyl-1,3,2-dioxaborolan-2-yl)-1H-pyrazole (276.92 mg, 1.43 mmol, 1 eq) and triphenylphosphine (449.19 mg, 1.71 mmol, 1.2 eq) in THF (3 mL), DBAD (394.34 mg, 1.71 mmol, 1.2 eq) was added dropwise The resulting mixture was stirred at 25° C. for 16 h. The reaction mixture was concentrated directly. The residue was purified by flash silica gel chromatography to give 5-methoxy-2-[[4-(4,4,5,5-tetramethyl-1,3,2-dioxaborolan-2-yl)pyrazol-1-yl]methyl]pyrimidine (380 mg, 37.9% yield) as a light yellow oil.

Step 2: A mixture of 5-methoxy-2-[[4-(4,4,5,5-tetramethyl-1,3,2-dioxaborolan-2-yl)pyrazol-1-yl]methyl]pyrimidine (110 mg, 173.96 μmol, 50% purity, 1 eq), Int 1-A (53.60 mg, 173.96 μmol, 1 eq), tripotassium phosphate (55.39 mg, 260.94 μmol, 1.5 eq) and Xphos Pd G3 (14.72 mg, 17.40 μmol, 0.1 eq) in dioxane (1 mL) and H₂O (0.2 mL) was degassed and purged with N₂ for 3 times, and then the mixture was stirred at 80° C. for 1 h under N₂ atmosphere. Water (5 mL) was added to the reaction mixture. The reaction mixture was then adjusted to pH˜3 by addition of aq. HCl (1 M). The resulting mixture was extracted with EtOAc (5 mL×3). The combined organic phase was washed with brine (5 mL), dried (anhydrous Na₂SO₄), filtered and concentrated. The residue was purified by prep-HPLC to give Example 7 (9.8 mg, 13.15% yield) as a white solid. LCMS: calc. for C₂₂H₁₉N₅O₄:417.1, found: [M+H]⁺418.3. ¹H NMR (400 MHZ, DMSO-d₆) δ 10.90 (s, 1H), 8.54 (s, 2H), 8.32 (s, 1H), 7.95 (s, 1H), 7.86 (s, 1H), 7.81 (s, 1H), 7.57-7.54 (m, 1H), 7.51-7.48 (m, 1H), 5.49 (s, 2H), 4.13 (dd, J=4.8, 12.0 Hz, 1H), 3.90 (s, 3H), 2.78-2.71 (m, 1H), 2.60 (br d, J=4.0 Hz, 1H), 2.34-2.30 (m, 1H), 2.13 (m, 1H).

### Examples 8A and 8B: Separation of Example 3

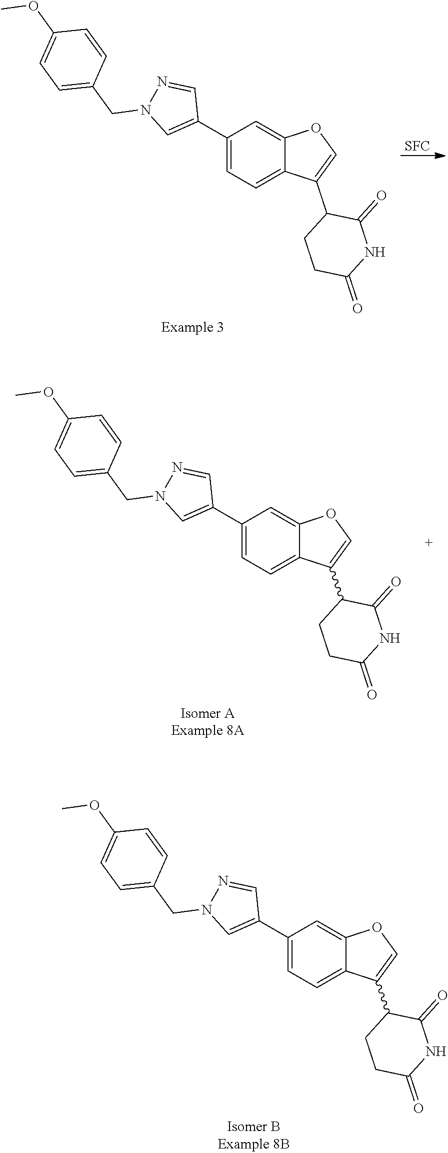 [SMILES: COc1ccc(Cn2cc(-c3ccc4c(C5CCC(=O)NC5=O)coc4c3)cn2)cc1 ; "SFC" ; COc1ccc(Cn2cc(-c3ccc4c(C5CCC(=O)NC5=O)coc4c3)cn2)cc1 ; COc1ccc(Cn2cc(-c3ccc4c(C5CCC(=O)NC5=O)coc4c3)cn2)cc1]

Example 3 (95 mg, 228.67 μmol, 1 eq) was purified by SFC (column: ChiralPak IH, 250*50 mm, 10 um; mobile phase: A: CO₂; B %: [EtOH/ACN], 50%-50%, 10 min) and most of solvent was removed under reduced pressure at 35° C., and the remaining solvent was removed by lyophilization to afford both isomer of 3-[6-[1-[(4-methoxyphenyl)methyl]pyrazol-4-yl]benzofuran-3-yl]piperidine-2,6-dione: isomer A as Example 8A, (44.6 mg, 107.36 μmol, 46.95% yield) as a white solid and isomer B as Example 8B (47.1 mg, 113.37 μmol, 49.58% yield) as a white solid.

Example 8A: LCMS: calc. for C₂₄H₂₁N₃O₄:415.2, found: [M+H]⁺416.3. ¹H NMR (400 MHZ, DMSO-d₆) δ:10.89 (s, 1H), 8.29 (s, 1H), 7.96 (s, 1H), 7.85 (s, 1H), 7.78 (s, 1H), 7.54 (d, J=8.0 Hz, 1H), 7.47 (d, J=8.0 Hz, 1H), 7.21-7.31 (m, 2H), 6.89-6.99 (m, 2H), 5.26 (s, 2H), 4.06-4.18 (m, 1H), 3.73 (s, 3H), 2.63-2.79 (m, 1H), 2.51-2.59 (m, 1H), 2.26-2.39 (m, 1H), 2.12-2.19 (m, 1H), e.e=98.42%.

Example 8B: LCMS: calc. for C₂₄H₂₁N₃O₄:415.2, found: [M+H]⁺416.4. ¹H NMR (400 MHZ, DMSO-d₆) δ:10.89 (s, 1H), 8.29 (s, 1H), 7.96 (s, 1H), 7.85 (s, 1H), 7.78 (s, 1H), 7.54 (d, J=8.0 Hz, 1H), 7.47 (d, J=8.0 Hz, 1H), 7.21-7.31 (m, 2H), 6.89-6.99 (m, 2H), 5.26 (s, 2H), 4.06-4.18 (m, 1H), 3.73 (s, 3H), 2.63-2.79 (m, 1H), 2.51-2.59 (m, 1H), 2.26-2.39 (m, 1H), 2.12-2.19 (m, 1H), e.e=92.42%.

### Example 9: N-[4-[[4-[3-(2,6-Dioxo-3-piperidyl)benzofuran-6-yl]pyrazol-1-yl]methyl]-2-methyl-phenyl]methanesulfonamide

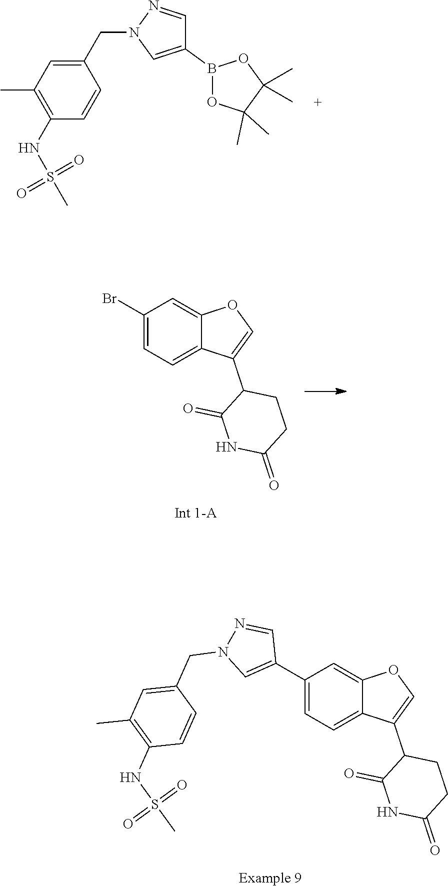

A mixture of N-[2-methyl-4-[[4-(4,4,5,5-tetramethyl-1,3,2-dioxaborolan-2-yl)pyrazol-1-yl]methyl]phenyl]methanesulfonamide (38.10 mg, 97.36 μmol, 1 eq), Int 1-A (30 mg, 97.36 μmol, 1 eq), XPhos Pd G3 (8.24 mg, 9.74 μmol, 0.1 eq) and K₃PO₄ (41.33 mg, 194.72 μmol, 2 eq) in THF (1 mL) and H₂O (0.1 mL) was degassed and purged with N₂ for 3 times, and then the mixture was stirred at 60° C. for 4 hours under N₂ atmosphere. The reaction mixture was concentrated under reduced pressure to give a residue. The residue was washed with DMF (2 mL) and the combined liquid was purified by prep-HPLC to afford Example 9 (17.0 mg, 34.26 μmol, 35.2% yield) as a white solid. LCMS: calc. for C₂₅H₂₄N₄O₅S: 492.1, found: [M+H]⁺493.2. ¹H NMR (400 MHZ, DMSO-d₆) δ 10.90 (s, 1H), 9.01 (s, 1H), 8.34 (s, 1H), 7.98 (s, 1H), 7.86 (s, 1H), 7.79 (s, 1H), 7.53-7.57 (m, 1H), 7.46-7.50 (m, 1H), 7.25 (d, J=8.0 Hz, 1H), 7.19 (s, 1H), 7.12 (dd, J=8.0, 1.6 Hz, 1H), 5.29 (s, 2H), 4.10-4.15 (m, 1H), 2.95 (s, 3H), 2.70-2.78 (m, 2H), 2.31-2.35 (m, 1H), 2.28 (s, 3H), 2.10-2.14 (m, 1H).

### Example 10:3-[6-[1-[(4-amino-3-methyl-phenyl)methyl]pyrazol-4-yl]benzofuran-3-yl]piperidine-2,6-dione

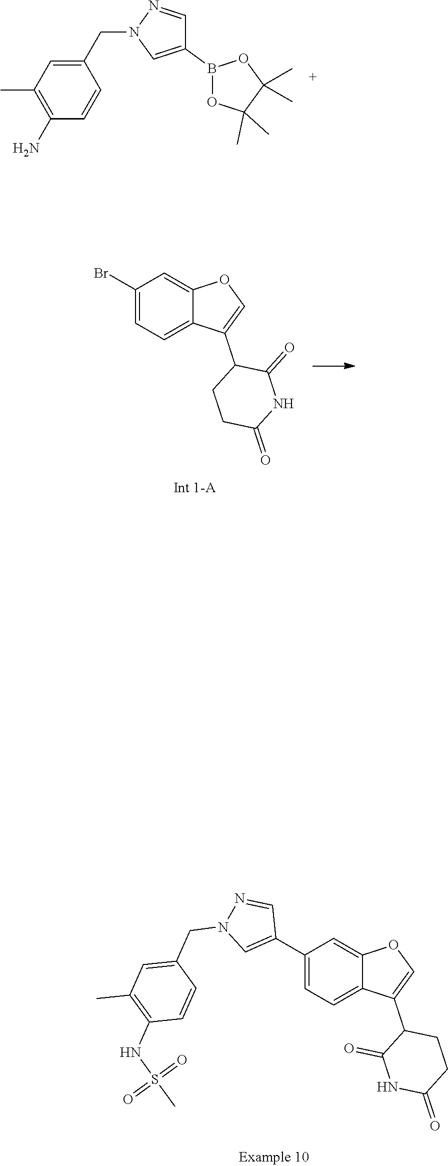

A mixture of 2-methyl-4-[4-(4,4,5,5-tetramethyl-1,3,2-dioxaborolan-2-yl)pyrazol-1-yl]methyl]aniline (30.49 mg, 97.36 μmol, 1 eq), Int 1-A (30 mg, 97.36 μmol, 1 eq), Pd(dppf)Cl₂ (7.12 mg, 9.74 μmol, 0.1 eq) and KF (11.31 mg, 194.72 μmol, 2 eq) in dioxane (1 mL) and H₂O (0.1 mL) was degassed and purged with N₂ for 3 times, and then the mixture was stirred at 85° C. for 16 hours under N₂ atmosphere. The reaction mixture was filtered directly and the filtrate was concentrated under reduced pressure to give a residue. The residue was washed with DMF (2 mL) and the combined liquid was purified by prep-HPLC to afford Example 10 (11.0 mg, 25.55 μmol, 26.2% yield) as a white solid. LCMS: calc. for C₂₄H₂₂N₄O₃:414.2, found: [M+H]⁺415.1. ¹H NMR (400 MHZ, DMSO-d₆) δ 10.89 (s, 1H), 8.20 (s, 1H), 7.92 (s, 1H), 7.84 (s, 1H), 7.77 (s, 1H), 7.51-7.54 (m, 1H), 7.43-7.48 (m, 1H), 6.88-6.95 (m, 2H), 6.56 (d, J=8.0 Hz, 1H), 5.09 (s, 2H), 4.85 (s, 2H), 4.10-4.14 (m, 1H), 2.71 (s, 1H), 2.59-2.60 (m, 1H), 2.25-2.31 (m, 1H), 2.10-2.15 (m, 1H), 2.02 (s, 3H).

### Example 11:4-[[4-[3-(2,6-dioxo-3-piperidyl)benzofuran-6-yl]pyrazol-1-yl]methyl]-N,N-dimethyl-benzenesulfonamide

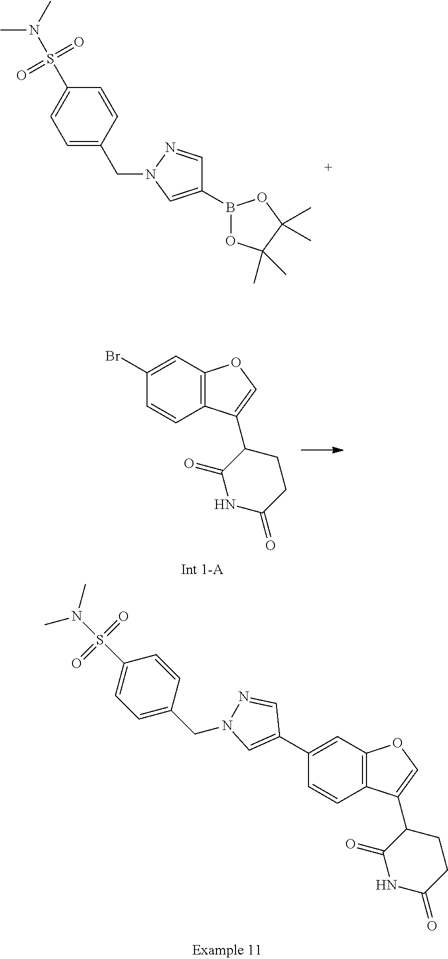

A mixture of N,N-dimethyl-4-[[4-(4,4,5,5-tetramethyl-1,3,2-dioxaborolan-2-yl)pyrazol-1-yl]methyl]benzenesulfonamide (58 mg, 148.23 μmol, 1 eq), Int 1-A (45.67 mg, 148.23 μmol, 1 eq), K₃PO₄ (47.20 mg, 222.34 μmol, 1.5 eq) and XPhos Pd G3 (12.55 mg, 14.82 μmol, 0.1 eq) in dioxane (3 mL) and H₂O (0.5 mL) was degassed and purged with N₂ for 3 times, and then the mixture was stirred at 80° C. for 1 h under N₂ atmosphere. The mixture was diluted with H₂O (50 mL), then was extracted with EtOAc (30 mL×3). The organic layer was washed with water (20 mL), dried over Na₂SO₄, filtered and concentrated under reduced pressure to give a residue. The residue was purified by prep-HPLC to afford Example 11 (12.7 mg, 17.40% yield) as a white solid. LCMS: calc. for C₂₅H₂₄N₄O₅S: 492.1, found: [M+H]⁺493.2. ¹HNMR (400 MHZ, DMSO-d₆)>10.90 (s, 1H), 8.41 (s, 1H), 8.05 (s, 1H), 7.85-7.88 (m, 1H), 7.82 (s, 1H), 7.75 (d, J=8.0 Hz, 2H), 7.54-7.59 (m, 1H), 7.49 (d, J=8.4 Hz, 3H), 5.50 (s, 2H), 4.13 (dd, J=12.0, 4.8 Hz, 1H), 2.70-2.80 (m, 1H), 2.59 (s, 6H), 2.54-2.58 (m, 1H), 2.30-2.36 (m, 1H), 2.09-2.18 (m, 1H).

### Example 12: 3-[6-[1-[1-(4-methoxyphenyl)ethyl]pyrazol-4-yl]benzofuran-3-yl]piperidine-2,6-dione

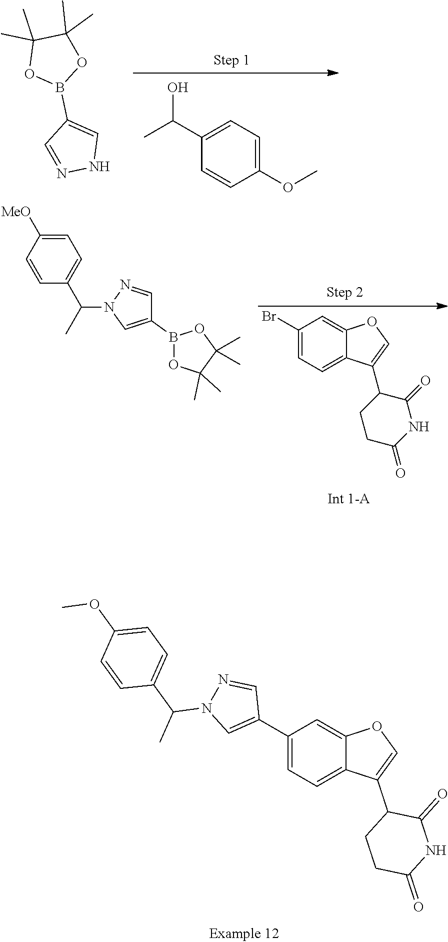

Step 1: To a solution of 1-(4-methoxyphenyl) ethanol (3 g, 19.71 mmol, 1 eq) and 4-(4,4,5,5-tetramethyl-1,3,2-dioxaborolan-2-yl)-1H-pyrazole (3.82 g, 19.71 mmol, 1 eq) in THF (20 mL) was added PPh3 (5.17 g, 19.71 mmol, 1 eq) and DIAD (3.99 g, 19.71 mmol, 3.83 mL, 1 eq). The mixture was stirred at 20° C. for 16 hours. The reaction mixture was poured into water (50 mL) and extracted with EtOAc (50 mL×2). The combined organic layer was washed with brine (20 mL), dried over anhydrous Na₂SO₄ and concentrated under reduced pressure to afford a residue. The residue was purified by column chromatography (SiO₂, Petroleum ether/Ethyl acetate=100/1 to 5/1) to afford 1-[1-(4-methoxyphenyl)ethyl]-4-(4,4,5,5-tetramethyl-1,3,2-dioxaborolan-2-yl)pyrazole (1.44 g, 3.54 mmol, 18.0% yield) as a colorless oil. LCMS: calc. for C₁₈H₂₅BN₂O₃: 328.2, found: [M+H]⁺328.7. ¹H NMR (400 MHz, DMSO-d₆) δ 7.97 (s, 1H), 7.58 (s, 1H), 7.23 (d, J=8.4 Hz, 2H), 6.85-6.90 (m, 2H), 5.52-5.59 (m, 1H), 3.72 (s, 3H), 1.77 (d, J=7.2 Hz, 3H), 1.23 (s, 12H).

Step 2: A mixture of Int 1-A (30 mg, 97.36 μmol, 1 eq), 1-[1-(4-methoxyphenyl)ethyl]-4-(4,4,5,5-tetramethyl-1,3,2-dioxaborolan-2-yl)pyrazole (31.95 mg, 97.36 μmol, 1 eq), Pd(dppf)Cl₂ (7.12 mg, 9.74 μmol, 0.1 eq) and KF (5.66 mg, 97.36 μmol, 1 eq) in dioxane (1 mL) and H₂O (0.1 mL) was degassed and purged with N₂ for 3 times, and then the mixture was stirred at 100° C. for 16 hours under N₂ atmosphere. The reaction mixture was filtered directly, and the filtrate was concentrated under reduced pressure to give a residue. The residue was purified by prep-HPLC to afford Example 12 (13.0 mg, 29.39 μmol, 30.2% yield) as a white solid. LCMS: calc. for C₂₅H₂₃N₃O₄:429.2, found: [M+H]⁺430.1. ¹H NMR (400 MHZ, DMSO-d₆) δ 10.89 (s, 1H), 8.35 (s, 1H), 7.95 (s, 1H), 7.85 (s, 1H), 7.79 (s, 1H), 7.45-7.59 (m, 2H), 7.25 (d, J=8.8 Hz, 2H), 6.89 (d, J=8.8 Hz, 2H), 5.54-5.56 (m, 1H), 4.10-4.14 (m, 1H), 3.72 (s, 3H), 2.70-2.74 (m, 1H), 2.54-2.56 (m, 1H), 2.30-2.34 (m, 1H), 2.04-2.18 (m, 1H), 1.82 (d, J=7.2 Hz, 3H).

### Example 13: 3-[6-[1-[1-(4-methoxyphenyl)-1-methyl-ethyl]pyrazol-4-yl]benzofuran-3-yl]piperidine-2,6-dione

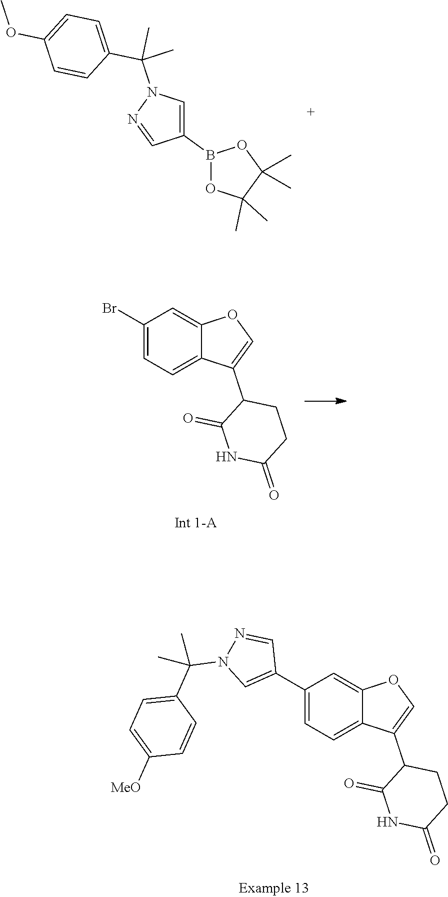

A mixture of 1-[1-(4-methoxyphenyl)-1-methyl-ethyl]-4-(4,4,5,5-tetramethyl-1,3,2-dioxaborolan-2-yl)pyrazole (39.99 mg, 116.83 μmol, 1.2 eq), 3-(6-bromobenzofuran-3-yl)piperidine-2,6-dione (30 mg, 97.36 μmol, 1 eq), Pd(dppf)Cl₂ (3.56 mg, 4.87 μmol, 0.05 eq) and KF (16.97 mg, 292.09 μmol, 3 eq) in dioxane (2 mL) and H₂O (0.2 mL) was degassed and purged with N₂ for 3 times, and then the mixture was stirred at 85° C. for 16 hours under N₂ atmosphere. The mixture was filtered and concentrated under reduced pressure to give a residue. The residue was purified by prep-HPLC to afford Example 13 (18.8 mg, 42.21 μmol, 43.4% yield) as a white solid. LCMS: calc. for C₂₆H₂₅N₃O₄:443.2, found: [M+Na]⁺466.1. ¹H NMR (400 MHZ, DMSO-d₆) δ 10.90 (s, 1H), 8.42 (s, 1H), 8.01 (s, 1H), 7.86 (s, 2H), 7.55 (s, 2H), 6.99 (d, J=8.8 Hz, 2H), 6.85 (d, J=8.8 Hz, 2H), 4.141-4.16 (m, 1H), 3.71 (s, 3H), 2.72-2.76 (m, 1H), 2.55-2.58 (m, 1H), 2.32-2.41 (m, 1H), 2.11-2.16 (m, 1H), 1.94 (s, 6H).

### Example 14:3-[6-[3-fluoro-1-[(4-methoxyphenyl)methyl]pyrazol-4-yl]benzofuran-3-yl]piperidine-2,6-dione

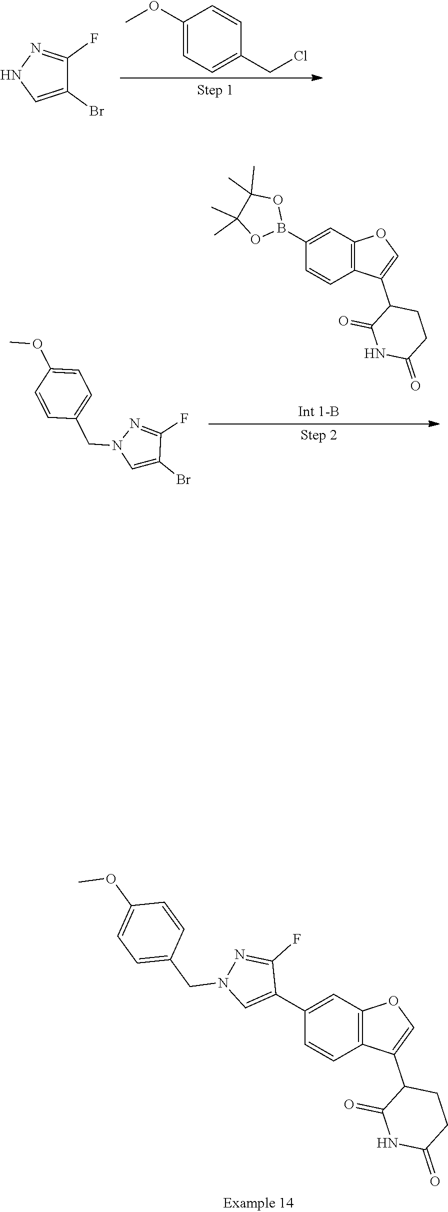

Step 1: To a solution of 4-bromo-3-fluoro-1H-pyrazole (100 mg, 606.19 μmol, 1 eq) in ACN(4 mL) was added 1-(chloromethyl)-4-methoxy-benzene (123.42 mg, 788.05 μmol, 1.3 eq) and K₂CO₃ (167.56 mg, 1.21 mmol, 2 eq) at 25° C. The mixture was stirred at 80° C. for 2 hr. The reaction mixture was concentrated. Water (10 mL) was added to the residue. The resulting mixture was extracted with DCM (10 mL×3). The combined organic phase was washed with brine (20 mL), dried over anhydrous Na₂SO₄, filtered and concentrated. The residue was purified by flash silica gel chromatography to give 4-bromo-3-fluoro-1-[(4-methoxyphenyl)methyl]pyrazole (80 mg, 46.29% yield) as a colorless oil. ¹H NMR (400 MHZ, CDCl₃) δ 7.25-7.19 (m, 2H), 7.16 (d, J=2.0 Hz, 1H), 6.93-6.88 (m, 2H), 5.05 (s, 2H), 3.82 (s, 3H).

Step 2: To a solution of 4-bromo-3-fluoro-1-[(4-methoxyphenyl)methyl]pyrazole (20 mg, 70.15 μmol, 1 eq), and Int 1-B (24.92 mg, 70.15 μmol, 1 eq) in dioxane (2 mL) and H₂O (0.5 mL) was added K₃PO₄ (29.78 mg, 140.30 μmol, 2 eq) and XPhos Pd G3 (5.94 mg, 7.01 μmol, 0.1 eq) under bubbled N₂ atmosphere. The mixture was stirred at 80° C. for 1 h. The residue was purified by prep-HPLC to give Example 14 (4.4 mg, 14.47% yield) as a white solid. LCMS: calc. for C₂₄H₂₀FN₃O₄:433.1, found: [M+H]⁺434.3. ¹H NMR (400 MHZ, DMSO-d₆) δ 10.91 (s, 1H), 8.32 (d, J=2.4 Hz, 1H), 7.90 (s, 1H), 7.69 (s, 1H), 7.60 (d, J=8.4 Hz, 1H), 7.44-7.38 (m, 1H), 7.29 (d, J=8.4 Hz, 2H), 6.93 (d, J=8.8 Hz, 2H), 5.14 (s, 2H), 4.14 (dd, J=12.0, 4.8 Hz, 1H), 3.74 (s, 3H), 2.81-2.69 (m, 1H), 2.64-2.53 (m, 1H), 2.33 (dq, J=12.4, 4.4 Hz, 1H), 2.18-2.06 (m, 1H). ¹⁹F NMR (376 MHZ, DMSO-d₆)₈-132.32.

### Example 15:3-[6-[1-[(4-morpholinophenyl)methyl]pyrazol-4-yl]benzofuran-3-yl]piperidine-2,6-dione

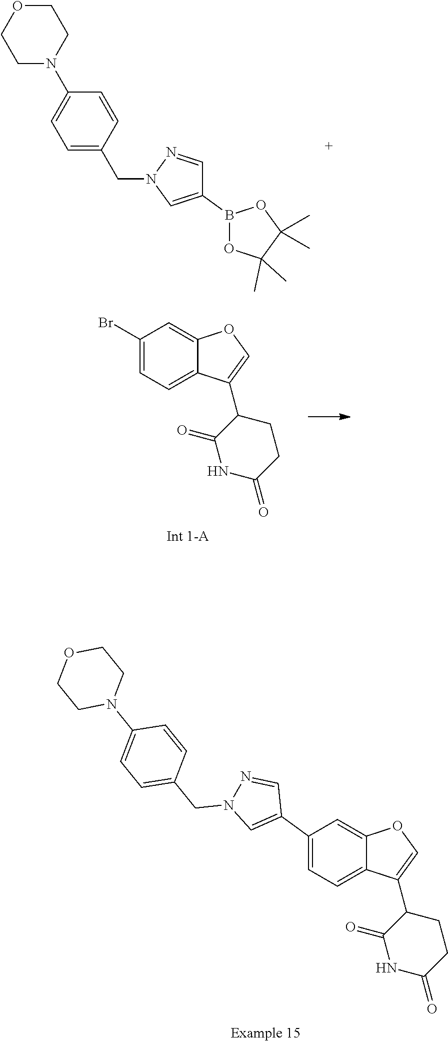

A mixture of 4-[4-[[4-(4,4,5,5-tetramethyl-1,3,2-dioxaborolan-2-yl)pyrazol-1-yl]methyl]phenyl]morpholine (40 mg, 54.16 μmol, 50% purity, 1 eq), Int 1-A (16.69 mg, 54.16 μmol, 1 eq), tripotassium phosphate (17.25 mg, 81.24 μmol, 1.5 eq), Xphos Pd G3 (4.58 mg, 5.42 μmol, 0.1 eq) in dioxane (0.5 mL) and H₂O (0.1 mL) was degassed and purged with N₂ for 3 times, and then the mixture was stirred at 80° C. for 1 h under N₂ atmosphere. Water (5 mL) was added to the reaction mixture. The reaction mixture was then adjusted to pH˜3 by aq. HCl (1 M). The resulting mixture was extracted with EtOAc (5 mL×3). The combined organic phase was washed with brine (5 mL), dried over anhydrous Na₂SO₄, filtered and concentrated. The residue was purified by prep-HPLC to give Example 15 (1.7 mg, 6.32% yield) as a white solid. LCMS: calc. for C₂₇H₂₆N₄O₄:470.2, found: [M+H]⁺471.3.

¹H NMR (400 MHZ, DMSO-d₆) δ 10.89 (s, 1H), 8.27 (s, 1H), 7.95 (s, 1H), 7.85 (s, 1H), 7.77 (s, 1H), 7.56-7.52 (m, 1H), 7.49-7.44 (m, 1H), 7.21 (d, J=8.0 Hz, 2H), 6.93 (d, J=8.4 Hz, 2H), 5.22 (s, 2H), 4.12 (dd, J=4.0, 12.0 Hz, 1H), 3.73-3.71 (m, 4H), 3.10-3.07 (m, 4H), 2.75-2.70 (m, 1H), 2.59 (m, 1H), 2.35-2.31 (m, 1H), 2.16-2.10 (m, 1H).

### Example 133: Diethyl(4-((4-(3-(2,6-dioxopiperidin-3-yl)benzofuran-6-yl)-1H-pyrazol-1-yl)methyl)phenyl)phosphonate

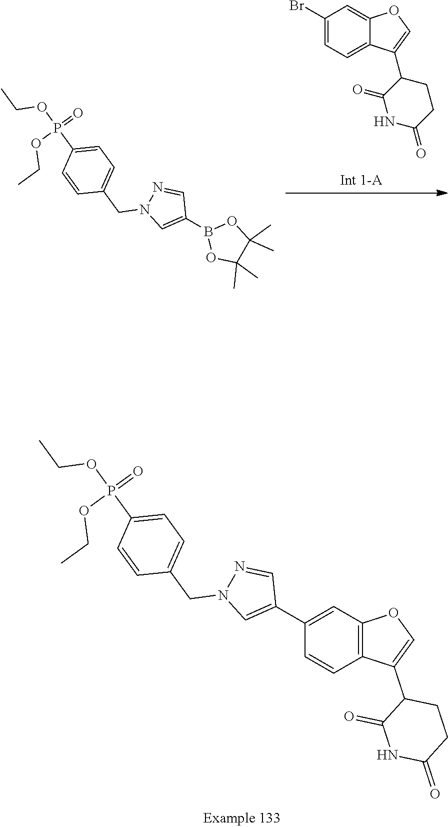

A mixture of 1-[(4-diethoxyphosphorylphenyl)methyl]-4-(4,4,5,5-tetramethyl-1,3,2-dioxaborolan-2-yl)pyrazole (160.0 mg, 380.7 μmol, 1 eq), Int 1-A (117.3 mg, 380.7 μmol, 1 eq), K₃PO₄ (121.2 mg, 571.1 μmol, 1.5 eq), XPhos Pd G3 (32.2 mg, 38.1 μmol, 0.1 eq) and H₂O (0.5 mL) in dioxane (2.5 mL) was degassed and purged with N₂ for 3 times, and then the mixture was stirred at 80° C. for 2 h under N₂ atmosphere. The mixture was diluted with H₂O (10 mL), extracted with EtOAc (10 mL×2). The organic layer was washed with brine (10 mL), dried over Na₂SO₄, filtered and concentrated. The residue was purified by prep-HPLC to give Example 133 (1 mg, 0.5% yield) as a white solid. ¹H NMR (400 MHZ, DMSO-d₆) § 10.89 (s, 1H), 8.38 (s, 1H), 8.03 (s, 1H), 7.86 (s, 1H), 7.81 (d, J=0.8 Hz, 1H), 7.70 (dd, J=12.0 Hz, 2H), 7.58-7.53 (m, 1H), 7.51-7.47 (m, 1H), 7.41 (dd, J=3.6, 12.0 Hz, 2H), 5.44 (s, 2H), 4.13 (dd, J=4.8, 12.0 Hz, 1H), 4.04-3.92 (m, 4H), 2.79-2.68 (m, 1H), 2.58 (td, J=3.6, 16.4 Hz, 1H), 2.38-2.29 (m, 1H), 2.13 (qd, J=4.4, 13.2 Hz, 1H), 1.21 (dd, J=7.2 Hz, 6H). LCMS: calc. for C₂₇H₂₈N₃O₆P: 521.2, found: [M+H]⁺522.2.

### Example 134:3-(6-(1-((5-(4-methylpiperazin-1-yl)pyridin-2-yl)methyl)-1H-pyrazol-4-yl)benzofuran-3-yl)piperidine-2,6-dione

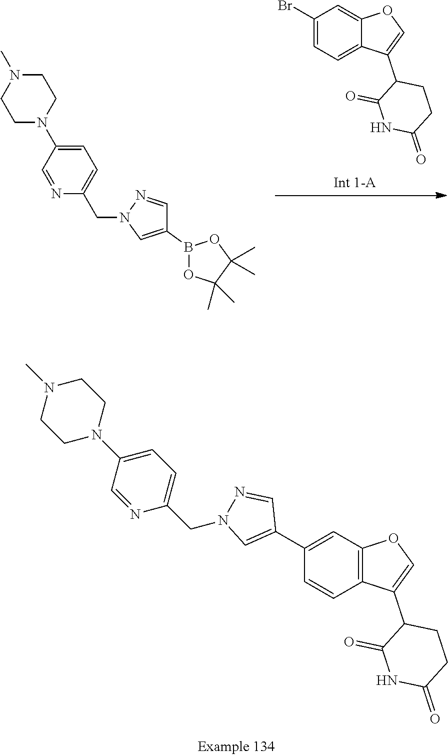 [SMILES: CN1CCN(c2ccc(Cn3cc(B4OC(C)(C)C(C)(C)O4)cn3)nc2)CC1 ; O=C1CCC(c2coc3cc(Br)ccc23)C(=O)N1 ; CN1CCN(c2ccc(Cn3cc(-c4ccc5c(C6CCC(=O)NC6=O)coc5c4)cn3)nc2)CC1]

To a solution of Int 1-A (24 mg, 77.89 μmol, 1 eq) and 1-methyl-4-[6-[[4-(4,4,5,5-tetramethyl-1,3,2-dioxaborolan-2-yl)pyrazol-1-yl]methyl]-3-pyridyl]piperazine (49.76 mg, 77.89 μmol, 60% purity, 1 eq) in dioxane (2 mL) and H₂O (0.5 mL) was added K₃PO₄ (33.07 mg, 155.78 μmol, 2 eq) and XPhos Pd G3 (6.59 mg, 7.79 μmol, 0.1 eq) under bubbled N₂ atmosphere. The mixture was stirred at 80° C. for 1 h. The reaction mixture was concentrated. The residue was purified by flash silica gel chromatography and dried by lyophilization to give Example 134 (1.1 mg, 2.27 μmol, 2.91% yield) as a brown solid. ¹H NMR (400 MHZ, MeOD) δ 8.25 (d, J=2.8 Hz, 1H), 8.13 (s, 1H), 7.92 (s, 1H), 7.70 (d, J=4.0 Hz, 2H), 7.57-7.45 (m, 2H), 7.41 (dd, J=2.8, 8.8 Hz, 1H), 7.16 (d, J=8.8 Hz, 1H), 5.60-5.50 (m, 1H), 5.39 (s, 2H), 4.13 (dd, J=4.8, 10.8 Hz, 1H), 3.37 (br s, 3H), 2.92 (br d, J=4.4 Hz, 4H), 2.83-2.67 (m, 2H), 2.57 (s, 4H), 2.47-2.26 (m, 2H). LCMS: calc. for C₂₇H₂₈N₆O₃:484.2, found: [M+H]⁺485.3.

### Example 135:3-(6-(1-((5-(2-oxa-6-azaspiro[3.3]heptan-6-yl)pyridin-2-yl)methyl)-1H-pyrazol-4-yl)benzofuran-3-yl)piperidine-2,6-dione

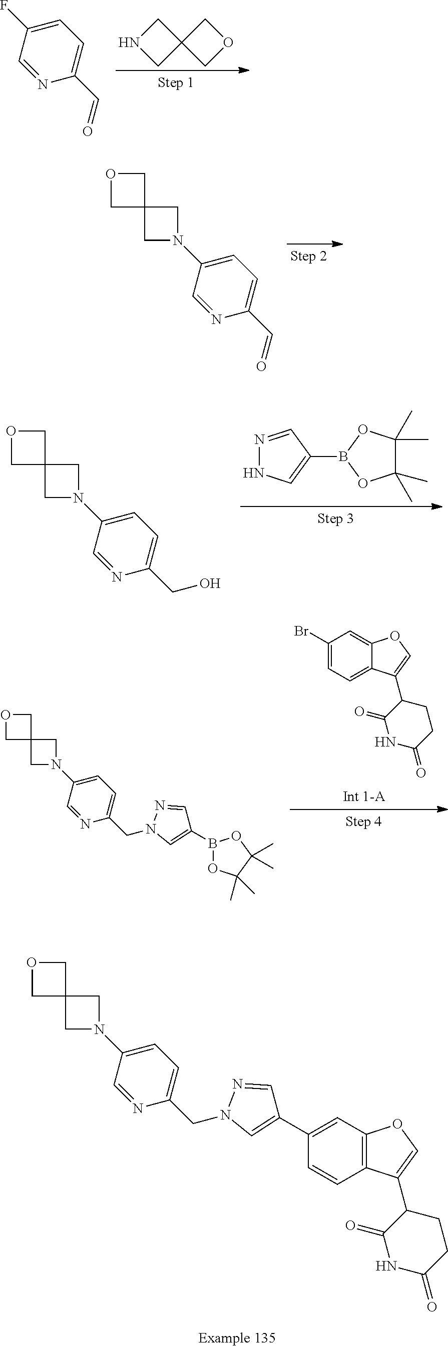

Step 1: To a solution of 5-fluoropyridine-2-carbaldehyde (3 g, 23.98 mmol, 1 eq) and 2-oxa-6-azaspiro[3.3]heptane (2.38 g, 23.98 mmol, 1 eq) in ACN(30 mL) was added K₂CO₃ (8.29 g, 59.95 mmol, 2.5 eq). The mixture was stirred at 80° C. for 1 h. The reaction mixture was concentrated. The residue was diluted with H₂O (30 mL) and extracted with DCM (40 mL×3). The combined organic layers were washed with brine (30 mL), dried over anhydrous Na₂SO₄, filtered and concentrated under reduced pressure to give a residue. The residue was purified by flash silica gel chromatography to give 5-(2-oxa-6-azaspiro[3.3]heptan-6-yl)pyridine-2-carbaldehyde (823 mg, 16.80% yield) as a light yellow solid. ¹H NMR (400 MHZ, CDCl₃) δ 9.87 (s, 1H), 7.88 (d, J=2.4 Hz, 1H), 7.82 (d, J=8.8 Hz, 1H), 6.72-6.69 (m, 1H), 4.87 (s, 4H), 4.24 (s, 4H).

Step 2: To a solution of 5-(2-oxa-6-azaspiro[3.3]heptan-6-yl)pyridine-2-carbaldehyde (800 mg, 3.92 mmol, 1 eq) in MeOH (10 mL) was added NaBH₄ (420 mg, 11.10 mmol, 2.83 eq) at 0° C., and the reaction was stirred at 0° C. for 1 h. The reaction was quenched with H₂O (30 mL) drop-wise. The resulting mixture was extracted with EtOAc (30 mL×3). The combined organic phase was washed with brine (20 mL), dried over anhydrous Na₂SO₄, filtered and concentrated. The residue was purified by flash silica gel chromatography to give [5-(2-oxa-6-azaspiro[3.3]heptan-6-yl)-2-pyridyl]methanol (645 mg, 79.84% yield) as a light yellow solid. ¹HNMR (400 MHZ, DMSO-d₆) δ 7.71 (d, J=2.8 Hz, 1H), 7.23 (d, J=8.4 Hz, 1H), 6.86-6.83 (m, 1H), 5.13 (t, J=5.6 Hz, 1H), 4.71 (s, 4H), 4.42 (d, J=5.6 Hz, 2H), 4.00 (s, 4H).

Step 3: A mixture of [5-(2-oxa-6-azaspiro[3.3]heptan-6-yl)-2-pyridyl]methanol (300 mg, 1.45 mmol, 1 eq), 4-(4,4,5,5-tetramethyl-1,3,2-dioxaborolan-2-yl)-1H-pyrazole (282.25 mg, 1.45 mmol, 1 eq), 2-(tributyl-phosphanylidene) acetonitrile (877.68 mg, 3.64 mmol, 2.5 eq) in toluene (6 mL) was degassed and purged with N₂ for 3 times, and then the mixture was stirred at 110° C. for 16 h under N₂ atmosphere. The reaction mixture was concentrated directly. The residue was purified by flash silica gel chromatography to give 6-[6-[4-(4,4,5,5-tetramethyl-1,3,2-dioxaborolan-2-yl)pyrazol-1-yl]methyl]-3-pyridyl]-2-oxa-6-azaspiro[3.3]heptane (300 mg, crude) as a dark brown liquid.

Step 4: A mixture of 6-[6-[4-(4,4,5,5-tetramethyl-1,3,2-dioxaborolan-2-yl)pyrazol-1-yl]methyl]-3-pyridyl]-2-oxa-6-azaspiro[3.3]heptane (140 mg, 73.25 μmol, 1 eq), Int 1-A (22.57 mg, 73.25 μmol, 1 eq), K₃PO₄ (23.32 mg, 109.87 μmol, 1.5 eq), Xphos Pd G3 (9.30 mg, 10.99 μmol, 0.15 eq) in dioxane (1 mL) and H₂O (0.2 mL) was degassed and purged with N₂ for 3 times, and then the mixture was stirred at 80° C. for 1 h under N₂ atmosphere. Water (5 mL) was added to the reaction mixture. The reaction mixture was then adjusted to pH˜3 by aq. HCl (1 M). The resulting mixture was extracted with EtOAc (10 mL×3). The combined organic phase was washed with brine (10 mL), dried over anhydrous Na₂SO₄, filtered and concentrated. The crude product was purified by reversed-phase HPLC to give Example 135 (1.2 mg, 3.19% yield) as a yellow solid. ¹HNMR (400 MHZ, DMSO-d₆) $10.90 (s, 1H), 8.28 (s, 1H), 7.95 (s, 1H), 7.85 (s, 1H), 7.80-7.78 (m, 1H), 7.77 (d, J=2.8 Hz, 1H), 7.56-7.52 (m, 1H), 7.49-7.46 (m, 1H), 7.07 (d, J=8.4 Hz, 1H), 6.85-6.82 (m, 1H), 5.28 (s, 2H), 4.70 (s, 4H), 4.16-4.10 (m, 1H), 4.02 (s, 4H), 2.79-2.68 (m, 1H), 2.61-2.55 (m, 1H), 2.35-2.27 (m, 1H), 2.16-2.08 (m, 1H). LCMS: calc. for C₂₇H₂₅N₅O₄:483.1, found: [M+H]⁺484.2.

### Example 136:3-[6-[1-[[6-(dimethylamino)-3-pyridyl]methyl]pyrazol-4-yl]benzofuran-3-yl]piperidine-2,6-dione

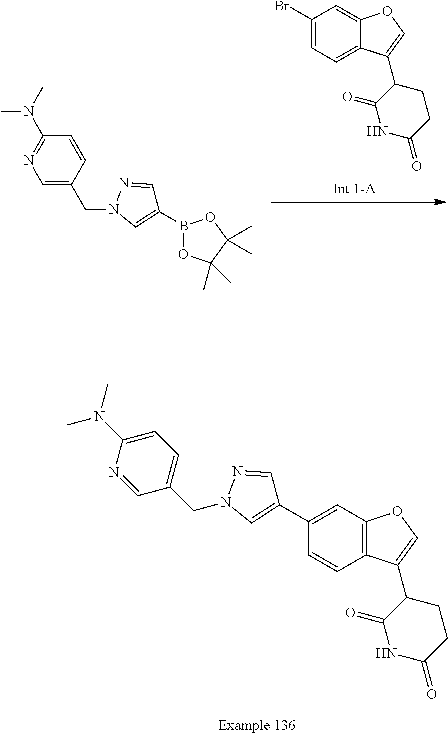

A mixture of N,N-dimethyl-5-[[4-(4,4,5,5-tetramethyl-1,3,2-dioxaborolan-2-yl)pyrazol-1-yl]methyl]pyridin-2-amine (109.56 mg, 116.83 μmol, 35% purity, 1.2 eq), Int 1-A (30 mg, 97.36 μmol, 1 eq), K₃PO₄ (41.33 mg, 194.72 μmol, 2 eq), XPhos Pd G3 (8.24 mg, 9.74 μmol, 0.1 eq) and in dioxane (2 mL) and H₂O (0.5 mL) was degassed and purged with N₂ for 3 times, and then the mixture was stirred at 80° C. for 1 h under N₂ atmosphere. The reaction mixture was concentrated directly. The residue was purified by prep-HPLC to give Example 136 (1.7 mg, 3.85 μmol, 3.95% yield, 97.25% purity) as a white solid. ¹HNMR (400 MHZ, DMSO-d₆) δ 10.90 (s, 1H), 8.27 (s, 1H), 8.13 (d, J=2.0 Hz, 1H), 7.94 (s, 1H), 7.85 (s, 1H), 7.77 (s, 1H), 7.59-7.38 (m, 3H), 6.61 (d, J=8.8 Hz, 1H), 5.17 (s, 2H), 4.12 (dd, J=4.8, 12.0 Hz, 1H), 2.99 (s, 6H), 2.80-2.68 (m, 1H), 2.63-2.54 (m, 1H), 2.32 (br dd, J=4.0, 12.4 Hz, 1H), 2.17-2.05 (m, 1H). LCMS: calc. for C₂₄H₂₃N₅O₃:429.1, found: [M+H]⁺430.3.

### Example 137:3-[6-[1-[dideuterio-(5-methoxypyrimidin-2-yl)methyl]pyrazol-4-yl]benzofuran-3-yl]piperidine-2,6-dione

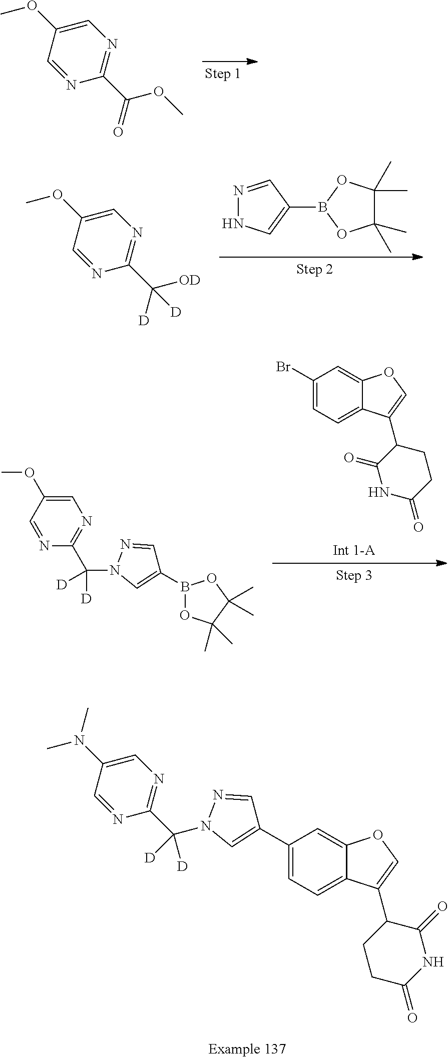

Step 1: To a solution of methyl 5-methoxypyrimidine-2-carboxylate (1.0 g, 5.95 mmol, 1 eq) in CH₃OD (10 mL) was added NaBD₄ (674.9 mg, 17.84 mmol, 3 eq) at 0° C. The resulting solution was stirred for 3 h at 25° C. The reaction was then quenched by the addition of D2O (3 mL). The resulting mixture was concentrated under vacuum to remove MeOD. The residue was purified by flash silica gel chromatography to give 2-[dideuterio (deuteriooxy)methyl]-5-methoxy-pyrimidine (330 mg, 38.63% yield) as a yellow solid. ¹H NMR (400 MHZ, DMSO-d₆) § 8.52 (s, 2H), 3.91 (s, 3H).

Step 2: To a solution of 2-[dideuterio (deuteriooxy)methyl]-5-methoxy-pyrimidine (150.0 mg, 1.05 mmol, 1 eq) and 4-(4,4,5,5-tetramethyl-1,3,2-dioxaborolan-2-yl)-1H-pyrazole (203.3 mg, 1.05 mmol, 1 eq) in THF (10 mL) was added PPh3 (329.7 mg, 1.26 mmol, 1.2 eq) and DBAD (289.5 mg, 1.26 mmol, 1.2 eq). The mixture was stirred at 25° C. for 16 h. The reaction mixture was concentrated. The residue was purified by flash silica gel chromatography to give 2-[dideuterio-[4-(4,4,5,5-tetramethyl-1,3,2-dioxaborolan-2-yl)pyrazol-1-yl]methyl]-5-methoxy-pyrimidine (75 mg, 3.98% yield) as a yellow oil. ¹H NMR (400 MHZ, DMSO-d₆) δ 8.55-8.45 (m, 4H), 3.89 (s, 3H), 1.41-1.35 (m, 12H). LCMS: calc. for C₁₅H₁₈D2BN₄O₃: 318.1, found: [M+H]⁺319.0.

Step 3: A solution of 2-[dideuterio-[4-(4,4,5,5-tetramethyl-1,3,2-dioxaborolan-2-yl)pyrazol-1-yl]methyl]-5-methoxy-pyrimidine (75.0 mg, 235.72 μmol, 1 eq), Int 1-A (72.6 mg, 235.72 μmol, 1 eq), K₃PO₄ (75.0 mg, 353.58 μmol, 1.5 eq) and XPhos Pd G3 (19.9 mg, 23.57 μmol, 0.1 eq) in dioxane (2.5 mL) and H₂O (0.5 mL) was degassed and purged with N₂ for 3 times, and then the mixture was stirred at 80° C. for 0.5 h under N₂ atmosphere. The residue was dissolved in H₂O (10 mL). The resulting suspension was extracted with EtOAc (10 mL×3). The combined organic phase was washed with H₂O (20 mL), dried over anhydrous Na₂SO₄, filtered and concentrated. The residue was purified by prep-HPLC to give Example 137 (14 mg, 11.38% yield) as a yellow solid. ¹H NMR (400 MHZ, DMSO-d₆) δ 10.91 (s, 1H), 8.54 (s, 2H), 8.33 (s, 1H), 7.96 (s, 1H), 7.86 (s, 1H), 7.80 (s, 1H), 7.57-7.53 (m, 1H), 7.52-7.47 (m, 1H), 4.13 (dd, J=4.8, 12.0 Hz, 1H), 3.89 (s, 3H), 2.77-2.70 (m, 1H), 2.61-2.55 (m, 1H), 2.33 (dd, J=3.6, 12.4 Hz, 1H), 2.16-2.10 (m, 1H). LCMS: calc. for C₂₂H₁₇D2N₅O₄:419.1, found: [M+H]⁺420.0.

### Example 138:3-(6-(1-((5-(methoxy-d₃)pyrimidin-2-yl)methyl)-1H-pyrazol-4-yl)benzofuran-3-yl)piperidine-2,6-dione

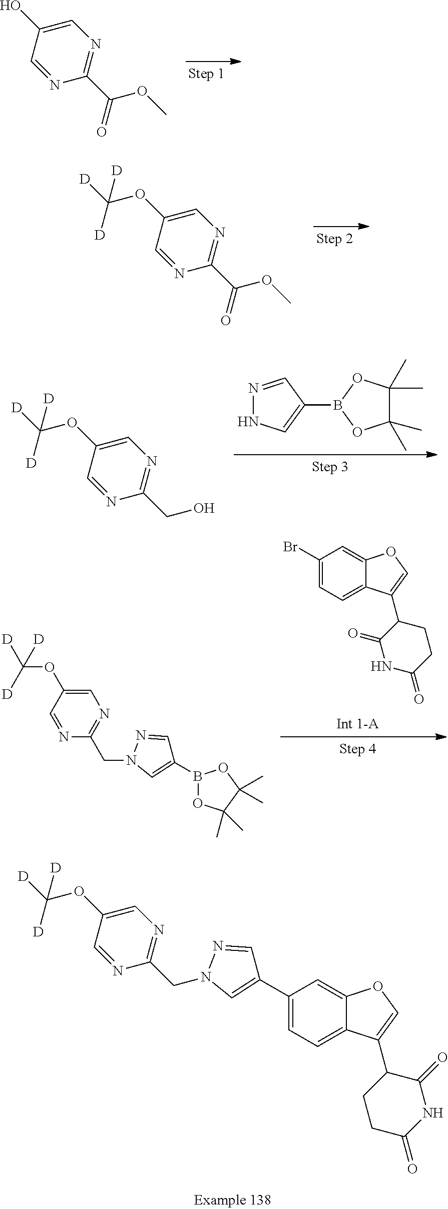

Step 1: A mixture of methyl 5-hydroxypyrimidine-2-carboxylate (500 mg, 3.24 mmol, 1 eq), K₂CO₃ (896.72 mg, 6.49 mmol, 2 eq), trideuterio (iodo) methane (920.95 mg, 6.49 mmol, 2 eq) in DMF (5 mL) was degassed and purged with N₂ for 3 times, and then the mixture was stirred at 25° C. for 16 h under N₂ atmosphere. The resulting mixture was diluted with H₂O (20 mL) extracted with EtOAc (30 mL×3). The combined organic phase was washed with brine (30 mL), dried over anhydrous Na₂SO₄, filtered and concentrated. The residue was purified by flash silica gel chromatography to give methyl 5-(trideuteriomethoxy)pyrimidine-2-carboxylate (410 mg, 73.83% yield) as a white and yellow solid. LCMS: calc. for C₇H₅D3N₂O₃:171.1, found: [M+H]⁺172.1.

Step 2: To a solution of methyl 5-(trideuteriomethoxy)pyrimidine-2-carboxylate (360 mg, 2.10 mmol, 1 eq) in MeOH (5 mL) was added NaBH₄ (290 mg, 7.67 mmol, 3.64 eq) at 0° C. The mixture was stirred at 25° C. for 3 h. The reaction was quenched with H₂O (5 mL) drop-wise. The reaction mixture was concentrated to give a residue. The residue was purified by flash silica gel chromatography to give [5-(trideuteriomethoxy)pyrimidin-2-yl]methanol (200 mg, 66.43% yield) as a yellow solid. LCMS: calc. for C₆H₅D3N₂O₂:143.1, found: [M+H]⁺144.1.

Step 3: To a solution of [5-(trideuteriomethoxy)pyrimidin-2-yl]methanol (100 mg, 698.53 μmol, 1 eq), 4-(4,4,5,5-tetramethyl-1,3,2-dioxaborolan-2-yl)-1H-pyrazole (149.10 mg, 768.38 μmol, 1.1 eq) and PPh3 (219.86 mg, 838.23 μmol, 1.2 eq) in THF (2 mL) was added DBAD (193.01 mg, 838.23 μmol, 1.2 eq). The mixture was stirred at 25° C. for 2 h under N₂ atmosphere. The reaction mixture was concentrated to give 2-[[4-(4,4,5,5-tetramethyl-1,3,2-dioxaborolan-2-yl)pyrazol-1-yl]methyl]-5-(trideuteriomethoxy)pyrimidine (200 mg, crude) as a colorless liquid. LCMS: calc. for C₁₅H₁₈D3BN ₄O₃: 319.2, found: [M+H]⁺319.9.

Step 4: A mixture of 2-[[4-(4,4,5,5-tetramethyl-1,3,2-dioxaborolan-2-yl)pyrazol-1-yl]methyl]-5-(trideuteriomethoxy)pyrimidine (200 mg, 626.60 μmol, 1 eq), Int 1-A (772.29 mg, 2.51 mmol, 4 eq), K₃PO₄ (199.51 mg, 939.91 μmol, 1.5 eq), XPhos Pd G3 (53.04 mg, 62.66 μmol, 0.1 eq) in dioxane (2 mL) and H₂O (0.4 mL) was degassed and purged with N₂ for 3 times, and then the mixture was stirred at 80° C. for 0.5 h under N₂ atmosphere. The reaction mixture was concentrated directly. The residue was purified by prep-HPLC and dried by lyophilization to give Example 138 (3.0 mg, 1.14% yield) as a white solid. ¹H NMR (400 MHZ, DMSO-d₆) § 10.90 (s, 1H), 8.54 (s, 2H), 8.32 (s, 1H), 7.95 (s, 1H), 7.86 (s, 1H), 7.81 (m, 1H), 7.57-7.53 (m, 1H), 7.51-7.47 (m, 1H), 5.49 (s, 2H), 4.13 (m, 1H), 2.80-2.69 (m, 1H), 2.63-2.59 (m, 1H), 2.39-2.32 (m, 1H), 2.16-2.10 (m, 1H). LCMS: calc. for C₂₂H₁₆D3N₅O₄:420.2, found: [M+H]⁺421.3.

### Example 139:3-(6-(1-((4-cyclopentylpyrimidin-2-yl)methyl)-1H-pyrazol-4-yl)benzofuran-3-yl)piperidine-2,6-dione

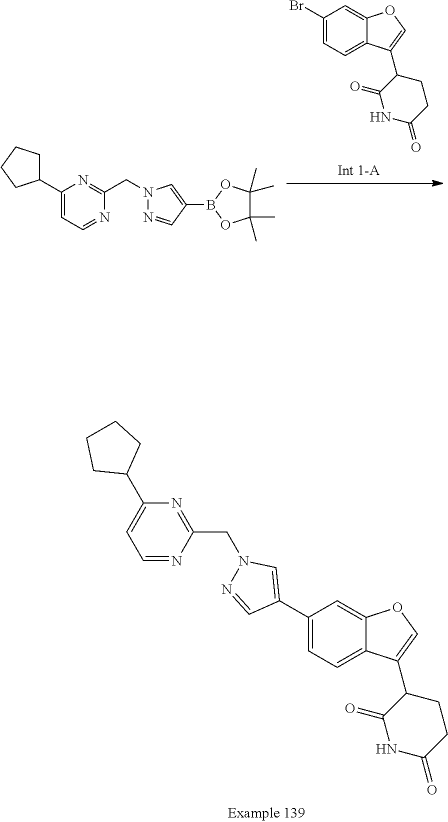 [SMILES: CC1(C)OB(c2cnn(Cc3nccc(C4CCCC4)n3)c2)OC1(C)C ; O=C1CCC(c2coc3cc(Br)ccc23)C(=O)N1 ; O=C1CCC(c2coc3cc(-c4cnn(Cc5nccc(C6CCCC6)n5)c4)ccc23)C(=O)N1]

A mixture of 5-cyclopentyl-2-[4-(4,4,5,5-tetramethyl-1,3,2-dioxaborolan-2-yl)pyrazol-1-yl]methyl]pyrimidine (91.98 mg, 259.63 μmol, 2 eq), Int 1-A (40 mg, 129.82 μmol, 1 eq), KF (11.31 mg, 194.72 μmol, 4.56 μL, 1.5 eq) and Pd(dppf) C₁₂ CH₂Cl₂ (10.60 mg, 12.98 μmol, 0.1 eq) in dioxane (2 mL) and H₂O (0.2 mL) was degassed and purged with N₂ for 3 times, and then the mixture was stirred at 90° C. for 1 h under N₂ atmosphere and microwave. The reaction mixture was poured into water (10 mL) and extracted with ethyl acetate (10 mL×3). The combined organic layer was washed with brine (20 mL), dried over anhydrous Na₂SO₄ and concentrated under reduced pressure to give a residue. The residue was purified by prep-TLC to give a crude product. The crude product was purified by prep-HPLC and lyophilization to afford Example 139 (1.2 mg, 2.63 μmol, 2.03% yield) as a white solid. ¹H NMR (400 MHZ, DMSO-d₆) δ 10.90 (s, 1H), 8.62 (d, J=5.2 Hz, 1H), 8.34 (s, 1H), 7.96 (s, 1H), 7.86 (s, 1H), 7.81 (s, 1H), 7.53-7.58 (m, 1H), 7.48-7.52 (m, 1H), 7.33 (d, J=5.2 Hz, 1H), 5.51 (s, 2H), 4.13 (dd, J=11.6, 4.8 Hz, 1H), 3.11-3.19 (m, 1H), 2.70-2.80 (m, 1H), 2.56-2.64 (m, 1H), 2.31-2.37 (m, 1H), 2.10-2.19 (m, 1H), 1.93-2.02 (m, 2H), 1.57-1.75 (m, 6H).

### Example 140:3-[6-[1-[(5-tetrahydropyran-2-ylpyrazin-2-yl)methyl]pyrazol-4-yl]benzofuran-3-yl]piperidine-2,6-dione

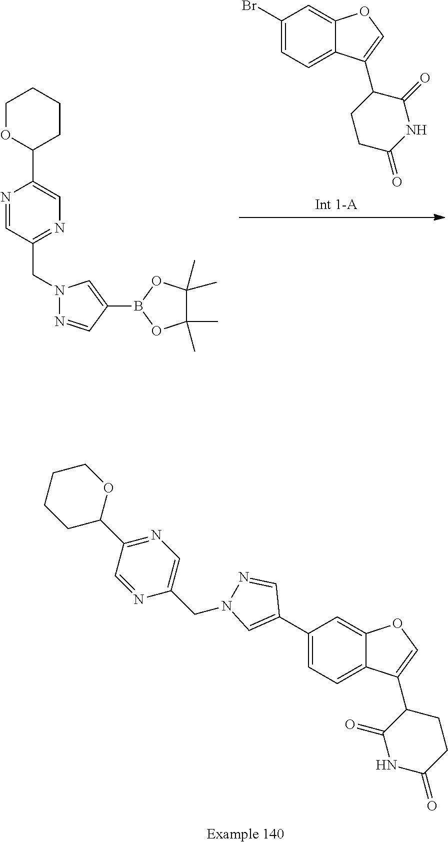 [SMILES: CC1(C)OB(c2cnn(Cc3cnc(C4CCCCO4)cn3)c2)OC1(C)C ; O=C1CCC(c2coc3cc(Br)ccc23)C(=O)N1 ; O=C1CCC(c2coc3cc(-c4cnn(Cc5cnc(C6CCCCO6)cn5)c4)ccc23)C(=O)N1]

A mixture of 2-tetrahydropyran-2-yl-5-[4-(4,4,5,5-tetramethyl-1,3,2-dioxaborolan-2-yl)pyrazol-1-yl]methyl]pyrazine (62.48 mg, 168.76 μmol, 1.3 eq), 3-(6-bromobenzofuran-3-yl)piperidine-2,6-dione (40 mg, 129.82 μmol, 1 eq), Pd(dppf) C₁₂ CH₂Cl₂ (106.01 mg, 129.82 μmol, 1 eq) and KF (7.54 mg, 129.82 μmol, 3.04 μL, 1 eq) in dioxane (2 mL) and H₂O (0.2 mL) was degassed and purged with N₂ for 3 times, and then the mixture was stirred at 90° C. for 1 h under N₂ atmosphere and microwave. The reaction mixture was poured into water (10 mL) and extracted with ethyl acetate (10 mL×3). The combined organic layers were washed with brine (20 mL), dried over anhydrous Na₂SO₄ and concentrated under reduced pressure to give a residue. The residue was purified by prep-TLC to give a crude product. The crude product was purified by prep-HPLC and lyophilized to afford Example 140 (19.6 mg, 41.57 μmol, 32.02% yield) as a yellow solid. ¹H NMR (400 MHz, DMSO-d₆) δ 10.91 (s, 1H), 8.64 (s, 1H), 8.49 (s, 1H), 8.40 (s, 1H), 8.01 (s, 1H), 7.86 (s, 1H), 7.81 (s, 1H), 7.53-7.60 (m, 1H), 7.47-7.52 (m, 1H), 5.52 (s, 2H), 4.48 (dd, J=11.2, 2.4 Hz, 1H), 4.02-4.08 (m, 2H), 3.53-3.61 (m, 1H), 2.70-2.79 (m, 1H), 2.55-2.61 (m, 1H), 2.28-2.37 (m, 1H), 2.08-2.16 (m, 1H), 1.91-1.97 (m, 1H), 1.84-1.90 (m, 1H), 1.61-1.69 (m, 1H), 1.53-1.59 (m, 2H), 1.42-1.52 (m, 1H). LCMS: calc. for C₂₆H₂₅N₅O₄:471.2, found: [M+H]⁺472.2.

### Examples 296 and 297:3-[6-(1-benzyl-3,6-dihydro-2H-pyridin-4-yl)benzofuran-3-yl]piperidine-2,6-dione (Example 296) and 3-[6-(1-benzyl-4-piperidyl)benzofuran-3-yl]piperidine-2,6-dione (Example 297)

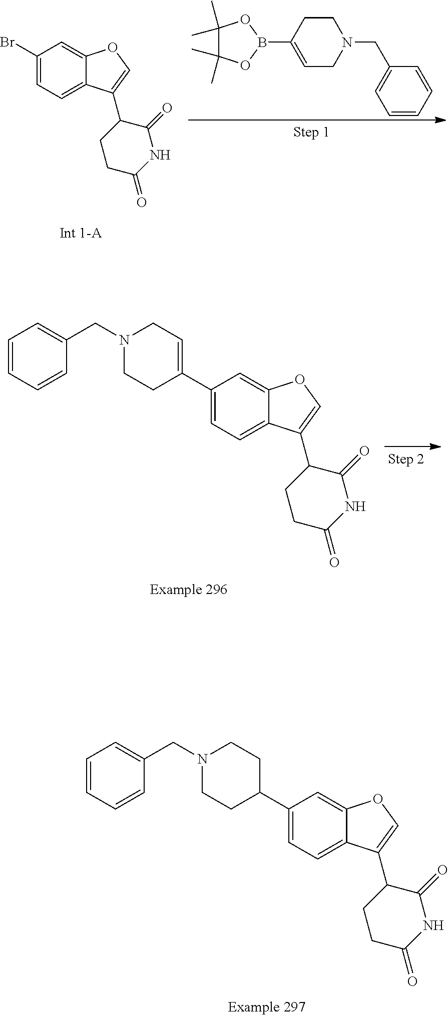 [SMILES: O=C1CCC(c2coc3cc(Br)ccc23)C(=O)N1 ; CC1(C)OB(C2=CCN(Cc3ccccc3)CC2)OC1(C)C ; O=C1CCC(c2coc3cc(C4=CCN(Cc5ccccc5)CC4)ccc23)C(=O)N1 ; O=C1CCC(c2coc3cc(C4CCN(Cc5ccccc5)CC4)ccc23)C(=O)N1]

Step 1: To a solution of Int 1-A (100 mg, 324.54 μmol, 1 eq), 1-benzyl-4-(4,4,5,5-tetramethyl-1,3,2-dioxaborolan-2-yl)-3,6-dihydro-2H-pyridine (106.82 mg, 357.00 μmol, 1.1 eq), Na₂CO₃ (103.19 mg, 973.62 μmol, 3 eq) in dioxane (5 mL) and H₂O (0.5 mL) was purged with N₂ for 3 times then added Pd(dppf)Cl₂ (11.87 mg, 16.23 μmol, 0.05 eq). The mixture was stirred at 100° C. for 16 hr under N₂ atmosphere. The mixture was poured into water (20 mL) and extracted with EtOAc (30 mL×2). The combined organic layers were washed with brine (20 mL×2), dried over Na₂SO₄, filtered and concentrated to give a residue. The residue was purified by prep-HPLC and dried by lyophilization to afford Example 296 (18 mg, 44.50 μmol, 89.10% yield, 99% purity) as a white solid. ¹H NMR (400 MHz, DMSO-d₆) δ 10.90 (s, 1H), 7.87 (s, 1H), 7.59 (s, 1H), 7.52 (d, J=8.4 Hz, 1H), 7.31-7.42 (m, 5H), 7.26-7.30 (m, 1H), 6.21 (s, 1H), 4.12 (dd, J=12.0, 4.8 Hz, 1H), 3.60 (s, 2H), 3.09 (d, J=2.4 Hz, 2H), 2.70-2.82 (m, 1H), 2.62-2.75 (m, J=5.6 Hz, 2H), 2.55-2.65 (m, 1H), 2.53-2.58 (m, 2H), 2.28-2.38 (m, 1H), 2.04-2.22 (m, 1H). LCMS: calc. for C₂₅H₂₄N₂O₃:400.2, found: [M+H]⁺401.2.

Step 2: To a solution of Example 296 (18 mg, 44.95 μmol, 1 eq) in EtOAc (20 mL) was added wet Pd/C (100 mg, 10% wt). The mixture was stirred at 25° C. for 1 hr under H₂ (15 psi). Crude LCMS showed desired product was detected. The reaction mixture was filtered and concentrated to give a residue. The residue was purified by prep-HPLC and dried by lyophilization to afford Example 297 (1.2 mg, 2.83 μmol, 6.30% yield, 95% purity) as a white solid. ¹H NMR (400 MHZ, CDCl₃) δ 8.02 (s, 1H), 7.53 (s, 1H), 7.34-7.43 (m, 6H), 7.30 (d, J=6.4 Hz, 1H), 7.17 (d, J=8.8 Hz, 1H), 3.99 (dd, J=8.8, 6.0 Hz, 1H), 3.55-3.69 (m, 2H), 3.08 (d, J=11.2 Hz, 2H), 2.71-2.85 (m, 2H), 2.65 (d, J=12.8 Hz, 1H), 2.33-2.41 (m, 2H), 2.10-2.26 (m, 2H), 1.75-2.00 (m, 4H). LCMS: calc. for C₂₅H₂₆N₂O₃:402.2, found: [M+H]⁺403.2.

### Example 298:3-[6-(1,3-benzothiazol-2-yl)benzofuran-3-yl]piperidine-2,6-dione

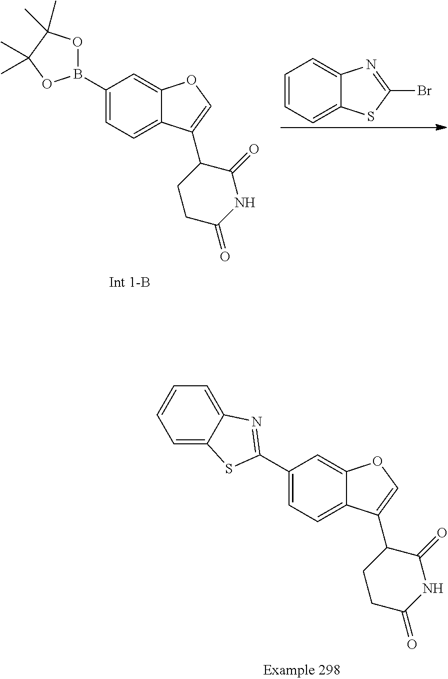 [SMILES: CC1(C)OB(c2ccc3c(C4CCC(=O)NC4=O)coc3c2)OC1(C)C ; Brc1nc2ccccc2s1 ; O=C1CCC(c2coc3cc(-c4nc5ccccc5s4)ccc23)C(=O)N1]

To a solution of Int 1-B (50 mg, 140.77 μmol, 1 eq) and 2-bromo-1,3-benzothiazole (30.14 mg, 140.77 μmol, 1 eq) in dioxane (2 mL) and H₂O (0.2 mL) was added KF (16.36 mg, 281.54 μmol, 6.60 μL, 2 eq) and Pd(dppf)Cl₂ (20.60 mg, 28.15 μmol, 0.2 eq). The mixture was bubbled by N₂ for 10 min. The mixture was stirred at 85° C. for 2 hr. The reaction mixture was concentrated under reduced pressure to give a residue. The residue was purified by prep-HPLC and dried by lyophilization to afford Example 298 (5.3 mg, 14.61 μmol, 10.38% yield, 99.87% purity) as an off-white solid. ¹H NMR (400 MHZ, DMSO-d₆) δ 10.94 (s, 1H), 8.30 (d, J=1.2 Hz, 1H), 8.16 (d, J=8.0 Hz, 1H), 8.05-8.12 (m, 2H), 8.01 (dd, J=8.0, 1.6 Hz, 1H), 7.79 (d, J=8.0 Hz, 1H), 7.53-7.59 (m, 1H), 7.44-7.50 (m, 1H), 4.23 (dd, J=12.4, 4.8 Hz, 1H), 2.74-2.82 (m, 1H), 2.59-2.65 (m, 1H), 2.35-2.41 (m, 1H), 2.12-2.20 (m, 1H). LCMS: calc. for C₂₀H₁₄N₂O₃S: 362.4, found: [M+H]⁺363.1.

### Examples 299 and 300:3-[6-[1-(4-methoxyphenyl)pyrazol-3-yl]methyl]benzofuran-3-yl]piperidine-2,6-dione (Example 299) and 3-[6-[[2-(4-methoxyphenyl)pyrazol-3-yl]methyl]benzofuran-3-yl]piperidine-2,6-dione (Example 300)

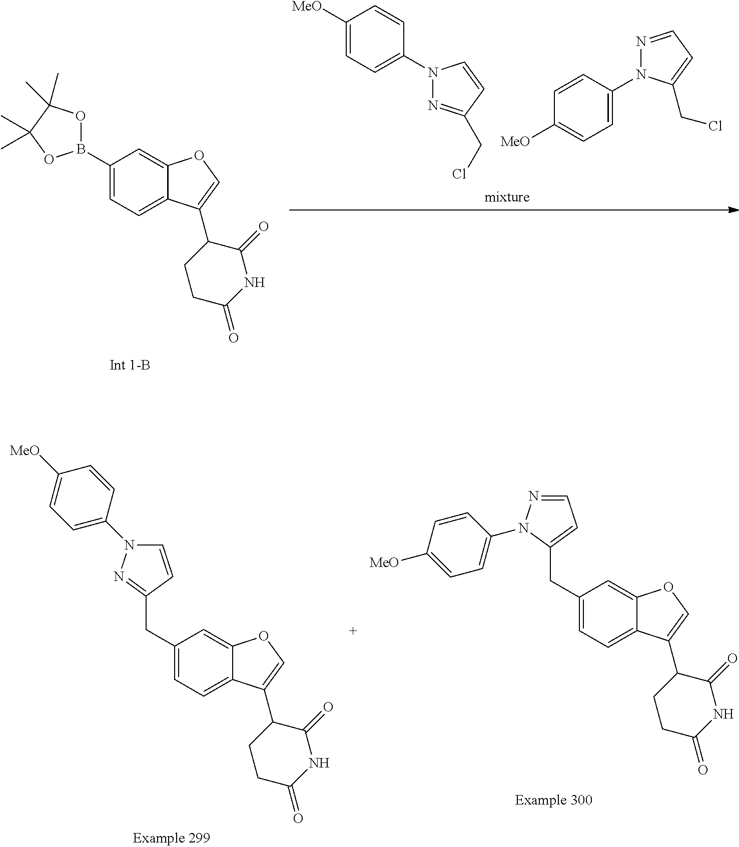

To a solution of 3-(chloromethyl)-1-(4-methoxyphenyl)pyrazole (20.00 mg, 89.82 μmol, 1 eq), Int 1-B (38.28 mg, 107.78 μmol, 1.2 eq), 5-(chloromethyl)-1-(4-methoxyphenyl)pyrazole (20.00 mg, 89.82 μmol, 1 eq) and Pd(dppf)Cl₂ CH₂Cl₂ (3.67 mg, 4.49 μmol, 0.05 eq) in dioxane (3 mL) and H₂O (0.3 mL) was added KF (10.44 mg, 179.64 μmol, 4.21 μL, 2 eq). The mixture was degressed with N₂ for 3 times and the mixture was stirred at 85° C. for 2 hr under N₂. Crude LCMS showed 65% of the desired MS was found. The mixture was diluted with EtOAc (15 mL×3), dried over anhydrous Na₂SO₄, filtered and concentrated under reduced pressure to afford the residue. The residue was purified by prep-HPLC and dried by lyophilization to afford Example 299 (15.3 mg, 36.61 μmol, 40.76% yield, 99.4% purity) as a white solid and Example 300 (2.5 mg, 5.58 μmol, 6.22% yield, 92.8% purity) as a white solid.

Example 299: 1H NMR (400 MHZ, DMSO-d₆) δ 10.90 (s, 1H), 8.25 (d, J=2.4 Hz, 1H), 7.84 (s, 1H), 7.70 (d, J=9.2 Hz, 2H), 7.46-7.52 (m, 2H), 7.19 (d, J=8.8 Hz, 1H), 7.02 (d, J=9.2 Hz, 2H), 6.29 (d, J=2.4 Hz, 1H), 4.10-4.14 (m, 1H), 4.09 (s, 2H), 3.78 (s, 3H), 2.71 (dd, J=12.0, 5.2 Hz, 1H), 2.57-2.61 (m, 1H), 2.31 (dd, J=12.4, 4.0 Hz, 1H), 2.08-2.11 (m, 1H). LCMS: calc. for C₂₄H₂₁N₃O₄:415.2, found: [M+H]⁺416.2; Example 300: 1H NMR (400 MHZ, DMSO-d₆) δ 10.89 (s, 1H), 7.84 (s, 1H), 7.53 (d, J=1.6 Hz, 1H), 7.47 (d, J=8.0 Hz, 1H), 7.37 (d, J=8.8 Hz, 2H), 7.25 (s, 1H), 7.04 (d, J=8.8 Hz, 2H), 6.98 (d, J=8.0 Hz, 1H), 6.12 (d, J=1.6 Hz, 1H), 4.11 (s, 2H), 4.09 (d, J=4.8 Hz, 1H), 3.80 (s, 3H), 2.66-2.71 (m, 1H), 2.56-2.59 (m, 1H), 2.29-2.34 (m, 1H), 2.05-2.15 (m, 1H). LCMS: calc. for C₂₄H₂₁N₃O₄:415.2, found: [M+H]⁺416.2.

Examples 16-70, 141-217, and 301-387 were prepared following the similar procedure used for preparing Examples 1-7, 9-13, and 15, starting from Int 1-A and their corresponding boronic acid or ester intermediates, or Example 14 and 296-300, starting from Int 1-B and their corresponding halides with modifications. Characterizations of Examples 16-70, 141-217, and 301-387 are provided in Table 1.

TABLE 1

| Example | | |
|---|---|---|
| ID | Structure | NMR/MS |
| 16 | 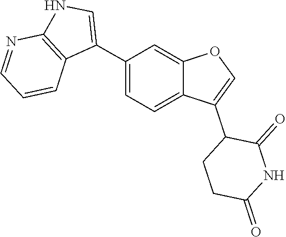 [SMILES: O=C1CCC(c2coc3cc(-c4c[nH]c5ncccc45)ccc23)C(=O)N1] | ¹H NMR (400 MHz, DMSO-d₆) δ 11.93 (s, 1H), 10.92 (brs, 1H), 8.26-8.37 (m, 2H), 7.83-7.96 (m, 3H), 7.63 (d, J = 0.8 Hz, 2H), 7.17 (dd, J = 8.0, 4.4 Hz, 1H), 4.17 (dd, J = 12.0, 4.8 Hz, 1H), 2.70-2.83 (m, 1H), 2.55-2.64 (m, 1H), 2.29-2.42 (m, 1H), 2.10-2.21 (m, 1H). LCMS: calc. for C₂₀H₁₅N₃O₃: 345.1, found: [M + H]⁺ 346.1 |
| 17 | 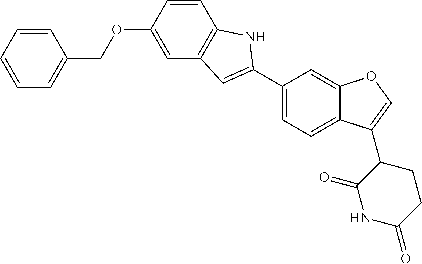 [SMILES: O=C1CCC(c2coc3cc(-c4cc5cc(OCc6ccccc6)ccc5[nH]4)ccc23)C(=O)N1] | ¹H NMR (400 MHz, DMSO-d₆) δ 11.43 (s, 1H), 10.93 (s, 1H), 8.04 (s, 1H), 7.94 (s, 1H), 7.75 (d, J = 8.4 Hz, 1H), 7.64 (d, J = 8.0 Hz, 1H), 7.45- 7.53 (m, 2H), 7.38-7.41 (m, 2H), 7.26-7.35 (m, 2H), 7.11 (s, 1H), 6.88 (s, 1H), 6.83 (dd, J = 8.8, 2.0 Hz, 1H), 5.11 (s, 2H), 4.17 (dd, J = 12.0, 4.8 Hz, 1H), 2.70-2.83 (m, 1H), 2.55-2.65 (m, 1H), 2.28-2.42 (m, 1H), 2.10-2.20 (m, 1H). LCMS: calc. for C₂₈H₂₂N₂O₄: 450.2, found: [M + H]⁺ 451.1. |
| 18 | 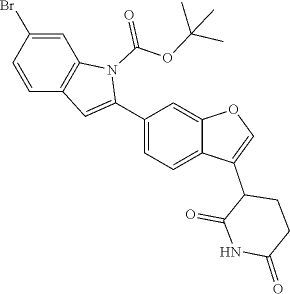 [SMILES: CC(C)(C)OC(=O)n1c(-c2ccc3c(C4CCC(=O)NC4=O)coc3c2)cc2ccc(Br)cc21] | ¹H NMR (400 MHz, DMSO-d₆) δ 10.91 (s, 1H), 8.27 (s, 1H), 7.97 (s, 1H), 7.70 (s, 1H), 7.64 (d, J = 8.0 Hz, 1H), 7.60 (d, J = 8.4 Hz, 1H), 7.44 (dd, J = 8.4, 1.6 Hz, 1H), 7.33 (dd, J = 8.0, 1.2 Hz, 1H), 6.79 (s, 1H), 4.19 (dd, J = 12.0, 4.8 Hz, 1H), 2.72-2.81 (m, 1H), 2.54-2.59 (m, 1H), 2.28-2.42 (m, 1H), 2.11-2.18 (m, 1H), 1.22 (s, 9H). LCMS: calc. for C₂₆H₂₃BrN₂O₅: 522.1, found: [M + Na]⁺ 545.1 (isotopic peak) |
| 19 | 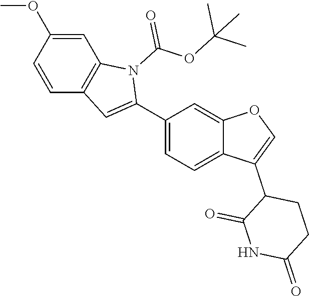 [SMILES: COc1ccc2cc(-c3ccc4c(C5CCC(=O)NC5=O)coc4c3)n(C(=O)OC(C)(C)C)c2c1] | ¹H NMR (400 MHz, DMSO-d₆) δ 10.92 (s, 1H), 7.95 (s, 1H), 7.66 (d, J = 2.0 Hz, 1H), 7.64 (s, 1H), 7.62 (d, J = 8.0 Hz, 1H), 7.51 (d, J = 8.8 Hz, 1H), 7.30 dd, J = 8.0, 1.6 Hz, 1H), 6.91 (dd, J = 8.4, 2.0 Hz, 1H), 6.69 (s, 1H), 4.18 (dd, J = 11.6, 4.4 Hz, 1H), 3.83 (s, 3H), 2.72-2.81 (m, 1H), 2.56-2.61 (m, 1H), 2.42-2.45 (m, 1H), 2.10-2.19 (m, 1H), 1.26 (s, 9H). LCMS: calc. for C₂₇H₂₆N₂O₆: 474.2, found: [M + H]⁺ 475.2. |
| 20 | 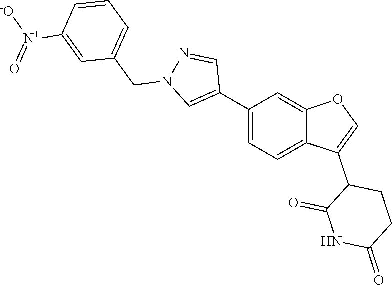 | ¹H NMR (400 MHz, DMSO-d₆) δ 10.90 (s, 1H), 8.42 (s, 1H), 8.14-8.20 (m, 2H), 8.05 (s, 1H), 7.86 (s, 1H), 7.81 (s, 1H), 7.72-7.77 (m, 1H), 7.65-7.70 (m, 1H), 7.54-7.58 (m, 1H), 7.47-7.51 (m, 1H), 5.53 (s, 2H), 4.09-4.16 (m, 1H), 2.69- 2.80 (m, 1H), 2.54-2.62 (m, 1H), 2.29-2.37 (m, 1H), 2.09-2.17 (m, 1H). LCMS: calc. for C₂₃H₁₈N₄O₅: 430.1, found: [M + H]⁺ 431.0 |
| 21 | 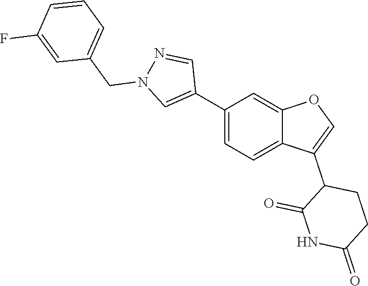 [SMILES: O=C1CCC(c2coc3cc(-c4cnn(Cc5cccc(F)c5)c4)ccc23)C(=O)N1] | ¹H NMR (400 MHz, DMSO-d₆) δ 10.90 (s, 1H), 8.37 (s, 1H), 8.02 (s, 1H), 7.86 (s, 1H), 7.81 (s, 1H), 7.53-7.57 (m, 1H), 7.47-7.51 (m, 1H), 7.38- 7.44 (m, 1H), 7.08-7.17 (m, 3H), 5.38 (s, 2H), 4.10-4.16 (m, 1H), 2.69-2.79 (m, 1H), 2.54-2.61 (m, 1H), 2.32-2.40 (m, 1H), 2.08-2.16 (m, 1H). LCMS: calc. for C₂₃H₁₈FN₃O₃: 403.1, found: [M + H]⁺ 404.2. |
| 22 | 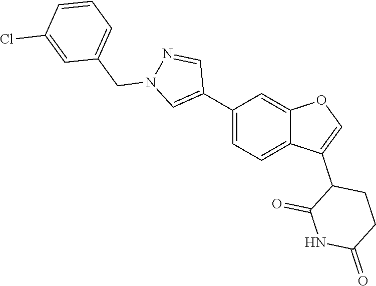 [SMILES: O=C1CCC(c2coc3cc(-c4cnn(Cc5cccc(Cl)c5)c4)ccc23)C(=O)N1] | ¹H NMR (400 MHz, DMSO-d₆) δ 10.90 (s, 1H), 8.38 (s, 1H), 8.02 (s, 1H), 7.86 (s, 1H), 7.81 (s, 1H), 7.54-7.57 (m, 1H), 7.47-7.51 (m, 1H), 7.34- 7.42 (m, 3H), 7.23-7.27 (m, 1H), 5.37 (s, 2H), 4.09-4.17 (m, 1H), 2.68-2.79 (m, 1H), 2.53-2.61 (m, 1H), 2.29-2.36 (m, 1H), 2.10-2.16 (m, 1H). LCMS: calc. for C₂₃H₁₈ClN₃O₃: 419.1, found: [M + H]⁺ 420.0 |
| 23 | 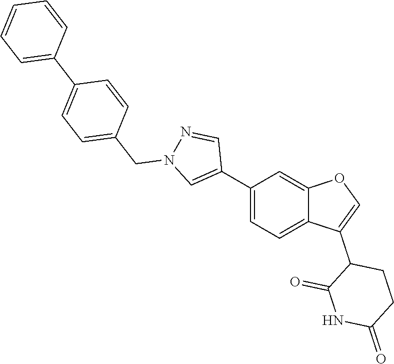 [SMILES: O=C1CCC(c2coc3cc(-c4cnn(Cc5ccc(-c6ccccc6)cc5)c4)ccc23)C(=O)N1] | ¹H NMR (400 MHz, DMSO-d₆) δ 10.90 (s, 1H), 8.39 (s, 1H), 8.01 (s, 1H), 7.86 (s, 1H), 7.81 (s, 1H), 7.63-7.66 (m, 4H), 7.54-7.57 (m, 1H), 7.43- 7.51 (m, 3H), 7.33-7.40 (m, 3H), 5.40 (s, 2H), 4.10-4.16 (m, 1H), 2.70-2.79 (m, 1H), 2.55-2.61 (m, 1H), 2.27-2.38 (m, 1H), 2.08-2.16 (m, 1H). LCMS: calc. for C₂₉H₂₃N₃O₃: 461.2, found: [M + H]⁺ 462.1 |
| 24 | 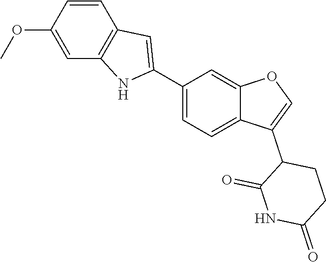 [SMILES: COc1ccc2cc(-c3ccc4c(C5CCC(=O)NC5=O)coc4c3)[nH]c2c1] | ¹H NMR (400 MHz, DMSO-d₆) δ 11.39 (s, 1H), 10.91 (s, 1H), 7.99 (s, 1H), 7.92 (s, 1H), 7.72 (dd, J = 8.4, 1.6 Hz, 1H), 7.60-7.65 (m, 1H), 7.40 (d, J = 8.4 Hz, 1H), 6.88 (d, J = 2.0 Hz, 2H), 6.64- 6.70 (m, 1H), 4.16 (dd, J = 11.6, 4.4 Hz, 1H), 3.79 (s, 3H), 2.69-2.82 (m, 1H), 2.56-2.64 (m, 1H), 2.39-2.41 (m, 1H), 2.35-2.37 (m, 1H). LCMS: LCMS: calc. for C₂₂H₁₈N₂O₄: 374.1, found: [M + H]⁺ 375.1. |
| 25 | 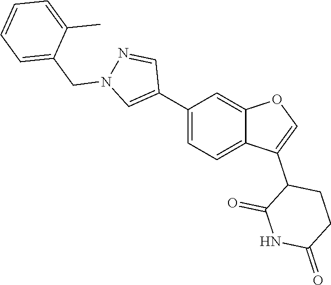 [SMILES: Cc1ccccc1Cn1cc(-c2ccc3c(C4CCC(=O)NC4=O)coc3c2)cn1] | ¹H NMR (400 MHz, DMSO-d₆) δ 10.90 (s, 1H), 8.26 (s, 1H), 8.00 (s, 1H), 7.85 (s, 1H), 7.80 (s, 1H), 7.47-7.56 (m, 2H), 7.14-7.22 (m, 3H), 7.01 (d, J = 7.2 Hz, 1H), 5.36 (s, 2H), 4.13-4.18 (m, 1H), 2.58-2.60 (m, 2H), 2.36-2.38 (m, 1H), 2.34 (s, 3H), 2.08-2.16 (m, 1H). LCMS: calc. for C₂₄H₂₁N₃O₃: 399.2, found: [M + H]⁺ 400.2 |
| 26 | 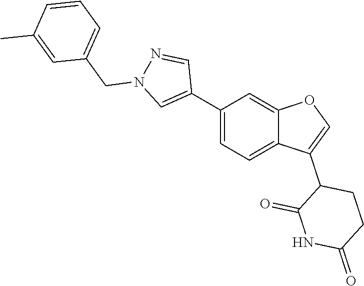 [SMILES: Cc1cccc(Cn2cc(-c3ccc4c(C5CCC(=O)NC5=O)coc4c3)cn2)c1] | ¹H NMR (400 MHz, DMSO-d₆) δ 10.90 (s, 1H), 8.26 (s, 1H), 8.00 (s, 1H), 7.85 (s, 1H), 7.80 (s, 1H), 7.47-7.56 (m, 2H), 7.14-7.22 (m, 3H), 7.01 (d, J = 7.2 Hz, 1H), 5.36 (s, 2H), 4.13-4.18 (m, 1H), 2.58-2.60 (m, 2H), 2.36-2.38 (m, 1H), 2.34 (s, 3H), 2.08-2.16 (m, 1H). LCMS: calc. for C₂₄H₂₁N₃O₃: 399.2, found: [M + H]⁺ 400.2 |
| 27 | 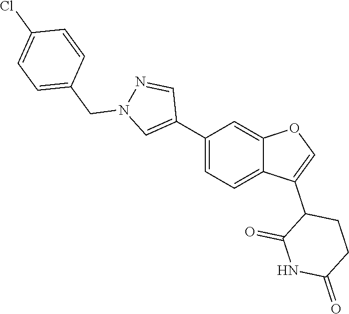 [SMILES: O=C1CCC(c2coc3cc(-c4cnn(Cc5ccc(Cl)cc5)c4)ccc23)C(=O)N1] | ¹H NMR (400 MHz, DMSO-d₆) δ 10.90 (s, 1H), 8.35 (s, 1H), 8.00 (s, 1H), 7.86 (s, 1H), 7.80 (s, 1H), 7.53-7.57 (m, 1H), 7.46-7.49 (m, 1H), 7.40- 7.45 (m, 2H), 7.28-7.33 (m, 2H), 5.35 (s, 2H), 4.13 (dd, J = 12.0, 4.8 Hz, 1H), 2.69-2.80 (m, 1H), 2.56-2.62 (m, 1H), 2.27-2.36 (m, 1H), 2.07- 2.16 (m, 1H). LCMS: calc. for C₂₃H₁₈ClN₃O₃: 419.1, found: [M + H]⁺ 419.9 |
| 28 | 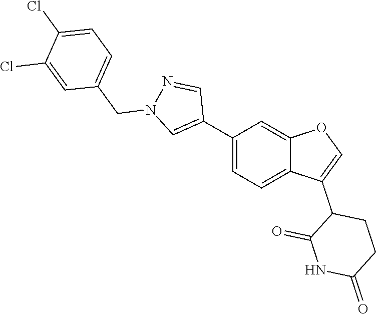 [SMILES: O=C1CCC(c2coc3cc(-c4cnn(Cc5ccc(Cl)c(Cl)c5)c4)ccc23)C(=O)N1] | ¹H NMR (400 MHz, DMSO-d₆) δ 10.90 (s, 1H), 8.37 (s, 1H), 8.02 (s, 1H), 7.86 (s, 1H), 7.80 (s, 1H), 7.63 (d, J = 8.4 Hz, 1H), 7.56 (d, J = 8.4 Hz, 2H), 7.46-7.51 (m, 1H), 7.26 (d, J = 8.4, 1H), 5.37 (s, 2H), 4.08-4.16 (m, 1H), 2.68-2.79 (m, 2H), 2.27-2.31 (m, 1H), 2.10-2.15 (m, 1H). LCMS: calc. for C₂₃H₁₇Cl₂N₃O₃: 453.1, found: [M + H]⁺ 453.8 |
| 29 | 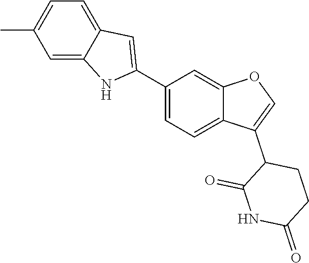 [SMILES: Cc1ccc2cc(-c3ccc4c(C5CCC(=O)NC5=O)coc4c3)[nH]c2c1] | ¹H NMR (400 MHz, DMSO-d₆) δ 11.39 (s, 1H), 10.93 (s, 1H), 8.04 (d, J = 1.2 Hz, 1H), 7.94 (s, 1H), 7.76 (dd, J = 8.0, 1.2 Hz, 1H), 7.64 (d, J = 8.0 Hz, 1H), 7.40 (d, J = 8.0 Hz, 1H), 7.18 (s, 1H), 6.90 (d, J = 1.2 Hz, 1H), 6.83 (dd, J = 8.0, 0.8 Hz, 1H), 4.16 (dd, J = 12.4, 4.8 Hz, 1H), 2.72-2.78 (m, 1H), 2.60-2.63 (m, 1H), 2.56-2.57 (m, 1H), 2.40 (s, 3H), 2.11-2.17 (m, 1H). LCMS: calc. for C₂₂H₁₈N₂O₃: 358.1, found: [M + H]⁺ 359.1. |
| 30 | 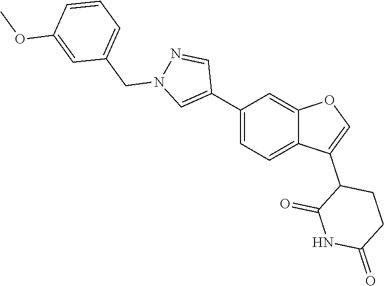 [SMILES: COc1cccc(Cn2cc(-c3ccc4c(C5CCC(=O)NC5=O)coc4c3)cn2)c1] | ¹H NMR (400 MHz, DMSO-d₆) δ 10.89 (s, 1H), 8.34 (s, 1H), 7.99 (s, 1H), 7.86 (s, 1H), 7.80 (s, 1H), 7.46-7.57 (m, 2H), 7.22-7.29 (m, 1H), 6.82- 6.89 (m, 3H), 5.31 (s, 2H), 4.08-4.17 (m, 1H), 3.73 (s, 3H), 2.70-2.79 (m, 1H), 2.54-2.58 (m, 1H), 2.26-2.38 (m, 1H), 2.08-2.17 (m, 1H). LCMS: calc. for C₂₄H₂₁N₃O₄: 415.2, found: [M + H]⁺ 415.9 |
| 31 | 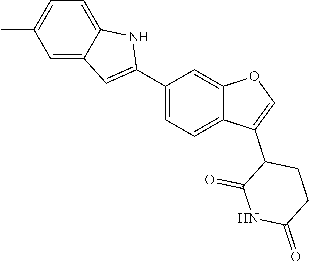 [SMILES: Cc1ccc2[nH]c(-c3ccc4c(C5CCC(=O)NC5=O)coc4c3)cc2c1] | ¹H NMR (400 MHz, DMSO-d₆) δ 11.39 (s, 1H), 10.93 (s, 1H), 8.04 (d, J = 1.2 Hz, 1H), 7.94 (s, 1H), 7.76 (dd, J = 8.0, 1.2 Hz, 1H), 7.64 (d, J = 8.0 Hz, 1H), 7.40 (d, J = 8.0 Hz, 1H), 7.18 (s, 1H), 6.90 (d, J = 1.2 Hz, 1H), 6.83 (dd, J = 8.0, 0.8 Hz, 1H), 4.16 (dd, J = 12.4, 4.8 Hz, 1H), 2.72-2.78 (m, 1H), 2.60-2.63 (m, 1H), 2.56-2.57 (m, 1H), 2.40 (s, 3H), 2.11-2.17 (m, 1H). LCMS: calc. for C₂₂H₁₈N₂O₃: 358.1, found: [M + H]⁺ 359.1. |
| 32 | 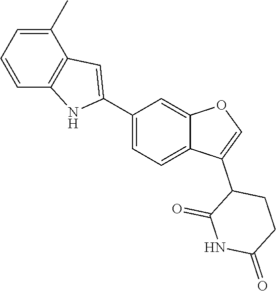 [SMILES: Cc1cccc2[nH]c(-c3ccc4c(C5CCC(=O)NC5=O)coc4c3)cc12] | ¹H NMR (400 MHz, DMSO-d₆) δ 11.54 (s, 1H), 10.94 (s, 1H), 8.08 (s, 1H), 7.94 (s, 1H), 7.81 (dd, J = 8.0, 1.2 Hz, 1H), 7.65 (d, J = 8.4 Hz, 1H), 7.22 (d, J = 8.0 Hz, 1H), 7.02 (s, 1H), 6.96-7.00 (m, 1H), 6.79 (d, J = 7.2 Hz, 1H), 4.17 (dd, J = 12.0, 4.8 Hz, 1H), 2.71-2.82 (m, 1H), 2.56-2.64 (m, 1H), 2.52 (s, 3H), 2.33-2.43 (m, 1H), 2.09- 2.19 (m, 1H). LCMS: calc. for C₂₂H₁₈N₂O₃: 358.1, found: [M + H]⁺ 359.2. |
| 33 | 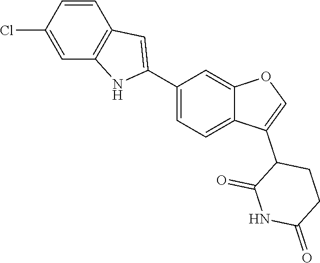 [SMILES: O=C1CCC(c2coc3cc(-c4cc5ccc(Cl)cc5[nH]4)ccc23)C(=O)N1] | ¹H NMR (400 MHz, DMSO-d₆) δ 11.77 (s, 1H), 10.95 (s, 1H), 8.08 (d, J = 1.2 Hz, 1H), 7.97 (s, 1H), 7.78 (dd, J = 8.4, 1.2 Hz, 1H), 7.68 (d, J = 8.0 Hz, 1H), 7.55 (d, J = 8.4 Hz, 1H), 7.41 (d, J = 1.2 Hz, 1H), 7.01-7.04 (m, 2H), 4.18 (dd, J = 12.0, 4.8 Hz, 1H), 2.71-2.83 (m, 1H), 2.55-2.65 (m, 1H), 2.31-2.41 (m, 1H), 2.11-2.19 (m, 1H). LCMS: calc. for C₂₁H₁₅ClN₂O₃: 378.1, found: [M + H]⁺ 379.1. |
| 34 | 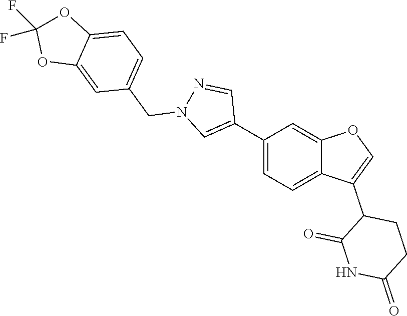 [SMILES: O=C1CCC(c2coc3cc(-c4cnn(Cc5ccc6c(c5)OC(F)(F)O6)c4)ccc23)C(=O)N1] | ¹H NMR (400 MHz, DMSO-d₆) δ 10.91 (s, 1H), 8.35 (s, 1H), 8.00 (s, 1H), 7.86 (s, 1H), 7.79 (s, 1H), 7.55 (d, J = 8.0 Hz, 1H), 7.47 (d, J = 7.6 Hz, 1H), 7.36-7.43 (m, 2H), 7.17 (d, J = 8.0 Hz, 1H), 5.36 (s, 2H), 4.09-4.16 (m, 1H), 2.64-2.87 (m, 1H), 2.54-2.62 (m, 1H), 2.25-2.37 (m, 1H), 2.08- 2.16 (m, 1H). LCMS: calc. for C₂₄H₁₇F₂N₃O₅: 465.1, found: [M + H]⁺ 466.2. |
| 35 | 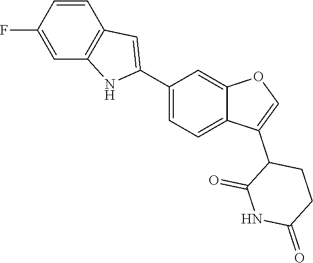 [SMILES: O=C1CCC(c2coc3cc(-c4cc5ccc(F)cc5[nH]4)ccc23)C(=O)N1] | ¹H NMR (400 MHz, DMSO-d₆) δ 11.71 (s, 1H), 10.94 (s, 1H), 8.05 (s, 1H), 7.95 (s, 1H), 7.76 (d, J = 8.4 Hz, 1H), 7.66 (d, J = 8.4 Hz, 1H), 7.52 (dd, J = 8.0, 5.6 Hz, 1H), 7.15 (d, J = 9.2 Hz, 1H), 6.99 (s, 1H), 6.87 (t, J = 8.4 Hz, 1H), 4.16 (dd, J = 11.6, 4.4 Hz, 1H), 2.70-2.82 (m, 1H), 2.56-2.65 (m, 1H), 2.33-2.40 (m, 1H), 2.08-2.17 (m, 1H). LCMS: calc. for C₂₁H₁₅FN₂O₃: 362.1, found: [M + H]⁺ 363.1. |
| 36 | 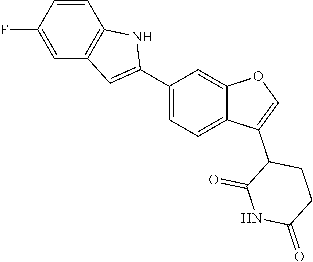 [SMILES: O=C1CCC(c2coc3cc(-c4cc5cc(F)ccc5[nH]4)ccc23)C(=O)N1] | ¹H NMR (400 MHz, DMSO-d₆) δ 11.69 (s, 1H), 10.95 (s, 1H), 8.07 (s, 1H), 7.96 (s, 1H), 7.78 (d, J = 8.4 Hz, 1H), 7.67 (d, J = 8.4 Hz, 1H), 7.38 (dd, J = 8.8, 4.4 Hz, 1H), 7.28 (dd, J = 9.6, 2.4 Hz, 1H), 6.87-7.02 (m, 2H), 4.17 (dd, J = 12.0, 4.8 Hz, 1H), 2.70-2.82 (m, 1H), 2.56-2.63 (m, 1H), 2.32-2.40 (m, 1H), 2.11-2.19 (m, 1H). LCMS: calc. for C₂₁H₁₅FN₂O₃: 362.1, found: [M + H]⁺ 363.1. |
| 37 | 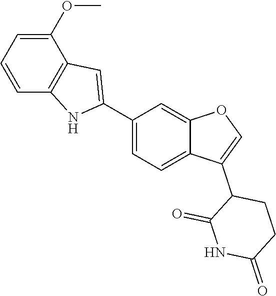 [SMILES: COc1cccc2[nH]c(-c3ccc4c(C5CCC(=O)NC5=O)coc4c3)cc12] | ¹H NMR (400 MHz, DMSO-d₆) δ 11.56 (d, J = 2.0 Hz, 1H), 10.93 (s, 1H), 8.05 (d, J = 1.2 Hz, 1H), 7.93 (s, 1H), 7.78 (dd, J = 8.4, 1.2 Hz, 1H), 7.63 (d, J = 8.4 Hz, 1H), 6.99-7.06 (m, 2H), 6.98 (d, J = 2.4 Hz, 1H), 6.51 (dd, J = 5.4, 3.2 Hz, 1H), 4.16 (dd, J = 12.0, 4.8 Hz, 1H), 3.89 (s, 3H), 2.71-2.81 (m, 1H), 2.56-2.63 (m, 1H), 2.34-2.37 (m, 1H), 2.10-2.18 (m, 1H). LCMS: calc. for C₂₂H₁₈N₂O₄: 374.1, found: [M + H]⁺ 375.1. |
| 38 | 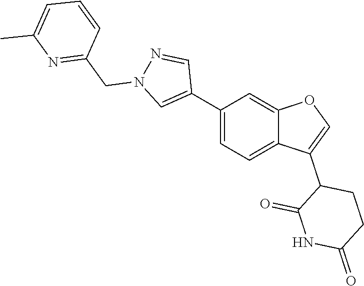 [SMILES: Cc1cccc(Cn2cc(-c3ccc4c(C5CCC(=O)NC5=O)coc4c3)cn2)n1] | ¹H NMR (400 MHz, DMSO-d₆) δ 10.90 (s, 1H), 8.42 (s, 0.8H, FA), 8.37 (s, 1H), 8.02 (s, 1H), 7.86 (s, 1H), 7.81 (s, 1H), 7.65 (t, J = 7.6 Hz, 1H), 7.53-7.58 (m, 1H), 7.47-7.52 (m, 1H), 7.18 (d, J = 7.6 Hz, 1H), 6.86 (d, J = 7.6 Hz, 1H), 5.42 (s, 2H), 4.13 (dd, J = 12.0, 4.8 Hz, 1H), 2.70-2.75 (m, 2H), 2.51-2.52 (m, 1H), 2.46 (s, 3H), 2.09- 2.16 (m, 1H). LCMS: calc. for C₂₃H₂₀N₄O₃: 400.2, found: [M + H]⁺ 401.0 |
| 39 | 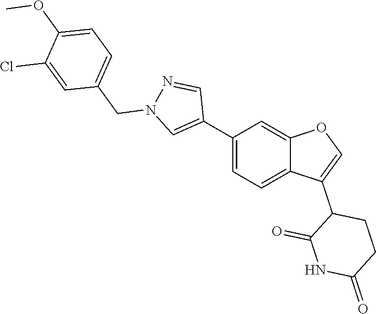 [SMILES: COc1ccc(Cn2cc(-c3ccc4c(C5CCC(=O)NC5=O)coc4c3)cn2)cc1Cl] | ¹H NMR (400 MHz, DMSO-d₆) δ 10.91 (brs, 1H), 8.33 (s, 1H), 7.99 (s, 1H), 7.86 (s, 1H), 7.79 (s, 1H), 7.52-7.58 (m, 1H), 7.45-7.51 (m, 1H), 7.40 (d, J = 2.0 Hz, 1H), 7.27 (dd, J = 8.4, 2.0 Hz, 1H), 7.13 (d, J = 8.4 Hz, 1H), 5.27 (s, 2H), 4.09-4.16 (m, 1H), 3.83 (s, 3H), 2.67-2.80 (m, 1H), 2.52-2.57 (m, 2H), 2.08-2.16 (m, 1H). LCMS: calc. for C₂₄H₂₀ClN₃O₄: 449.1, found: [M + H]⁺ 450.1 |
| 40 | 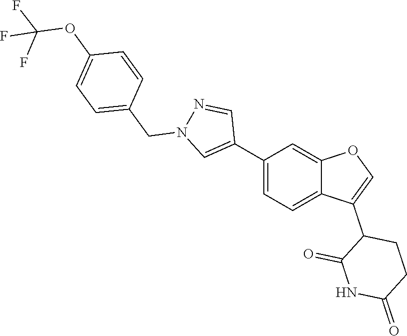 [SMILES: O=C1CCC(c2coc3cc(-c4cnn(Cc5ccc(OC(F)(F)F)cc5)c4)ccc23)C(=O)N1] | ¹H NMR (400 MHz, DMSO-d₆) δ 10.91 (s, 1H), 8.38 (s, 1H), 8.02 (s, 1H), 7.86 (s, 1H), 7.80 (s, 1H), 7.53-7.58 (m, 1H), 7.46-7.51 (m, 1H), 7.34- 7.43 (m, 4H), 5.40 (s, 2H), 4.13 (dd, J = 12.0, 4.8 Hz, 1H), 2.69-2.79 (m, 1H), 2.56 (d, J = 4.0 Hz, 1H), 2.30-2.36 (m, 1H), 2.07-2.17 (m, 1H). LCMS: calc. for C₂₄H₁₈F₃N₃O₄: 469.1, found: [M + H]⁺ 470.1 |
| 41 | 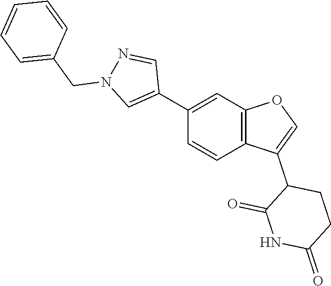 [SMILES: O=C1CCC(c2coc3cc(-c4cnn(Cc5ccccc5)c4)ccc23)C(=O)N1] | ¹H NMR (400 MHz, DMSO-d₆) δ 10.88 (s, 1H), 8.34 (s, 1H), 7.99 (s, 1H), 7.85 (s, 1H), 7.79 (s, 1H), 7.53-7.57 (m, 1H), 7.45-7.51 (m, 1H), 7.33- 7.39 (m, 2H), 7.26-7.32 (m, 3H), 5.35 (s, 2H), 4.08-4.16 (m, 1H), 2.69-2.79 (m, 1H), 2.56-2.62 (m, 1H), 2.27-2.38 (m, 1H), 2.09-2.17 (m, 1H). LCMS: calc. for C₂₃H₁₉N₃O₃: 385.1, found: [M + H]⁺ 386.0 |
| 42 | 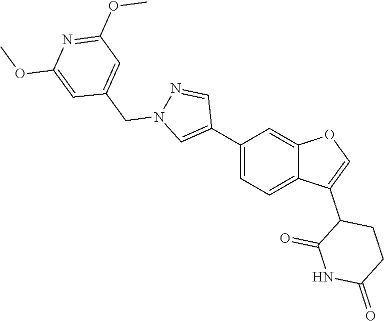 [SMILES: COc1cc(Cn2cc(-c3ccc4c(C5CCC(=O)NC5=O)coc4c3)cn2)cc(OC)n1] | ¹H NMR (400 MHz, DMSO-d₆) δ 10.89 (s, 1H), 8.35 (s, 1H), 8.03 (s, 1H), 7.86 (s, 1H), 7.81 (s, 1H), 7.45-7.64 (m, 2H), 6.18 (s, 2H), 5.31 (s, 2H), 4.08-4.17 (m, 1H), 3.82(s, 6H), 2.70-2.80 (m, 1H), 2.54-2.63 (m, 1H), 2.25-2.40 (m, 1H), 2.04-2.18 (m, 1H). LCMS: calc. for C₂₄H₂₂N₄O₅: 446.2, found: [M + H]⁺ 447.0 |
| 43 | 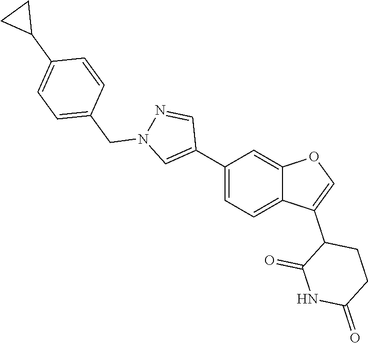 [SMILES: O=C1CCC(c2coc3cc(-c4cnn(Cc5ccc(C6CC6)cc5)c4)ccc23)C(=O)N1] | ¹H NMR (400 MHz, DMSO-d₆) δ 10.89 (s, 1H), 8.37 (s, 0.2H, FA), 8.30 (s, 1H), 7.96 (s, 1H), 7.85 (s, 1H), 7.78 (s, 1H), 7.52-7.56 (m, 1H), 7.45-7.49 (m, 1H), 7.18 (d, J = 8.0 Hz, 2H), 7.05 (d, J = 8.4 Hz, 2H), 5.27 (s, 2H), 4.09-4.15 (m, 1H), 2.69-2.78 (m, 1H), 2.55-2.61 (m, 1H), 2.30- 2.34 (m, 1H), 2.09-2.15 (m, 1H), 1.86-1.92 (m, 1H), 0.90-0.95 (m, 2H), 0.61-0.66 (m, 2H). LCMS: calc. for C₂₆H₂₃N₃O₃: 425.2, found: [M + H]⁺ 426.1 |
| 44 | 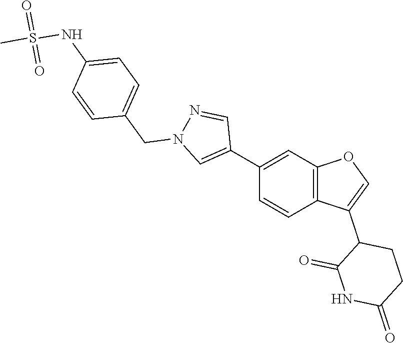 [SMILES: CS(=O)(=O)Nc1ccc(Cn2cc(-c3ccc4c(C5CCC(=O)NC5=O)coc4c3)cn2)cc1] | ¹H NMR (400 MHz, DMSO-d₆) δ 10.91 (s, 1H), 9.77 (s, 1H), 8.32 (s, 1H), 7.98 (s, 1H), 7.86 (s, 1H), 7.79 (s, 1H), 7.55 (d, J = 8.0 Hz, 1H), 7.48 (d, J = 8.0 Hz, 1H), 7.29 (d, J = 8.4 Hz, 2H), 7.19 (d, J = 8.4 Hz, 2H), 5.29 (s, 2H), 4.12 (dd, J = 12.0, 4.4 Hz, 1H), 2.97 (s, 3H), 2.68-2.82 (m, 1H), 2.50-2.61 (m, 1H), 2.26-2.40 (m, 1H), 2.08- 2.20 (m, 1H). LCMS: calc. for C₂₄H₂₂N₄O₅S: 478.1, found: [M + H]⁺ 479.2 |
| 45 | 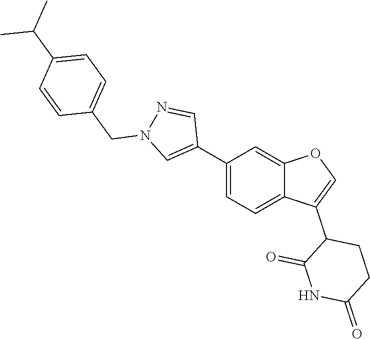 [SMILES: CC(C)c1ccc(Cn2cc(-c3ccc4c(C5CCC(=O)NC5=O)coc4c3)cn2)cc1] | ¹H NMR (400 MHz, DMSO-d₆) δ 10.89 (s, 1H), 8.33 (s, 1H), 7.97 (s, 1H), 7.85 (s, 1H), 7.79 (s, 1H), 7.53-7.56 (m, 1H), 7.46-7.49 (m, 1H), 7.22 (s, 4H), 5.29 (s, 2H), 4.12 (dd, J = 12.0, 4.8 Hz, 1H), 2.72-2.93 (m, 1H), 2.69-2.76 (m, 1H), 2.55- 2.61 (m, 1H), 2.25-2.39 (m, 1H), 2.09-2.15 (m, 1H), 1.17 (d, J = 7.2 Hz, 6H). LCMS: calc. for C₂₆H₂₅N₃O₃: 427.2, found: [M + H]⁺ 428.4 |
| 46 | 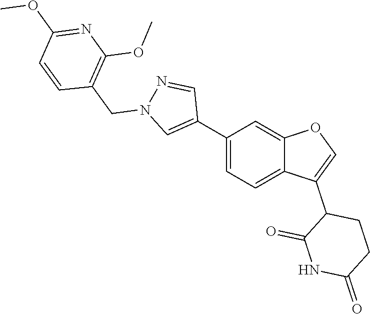 [SMILES: COc1ccc(Cn2cc(-c3ccc4c(C5CCC(=O)NC5=O)coc4c3)cn2)c(OC)n1] | ¹H NMR (400 MHz, DMSO-d₆) δ 10.89 (s, 1H), 8.21 (s, 1H), 7.95 (s, 1H), 7.76-7.88 (m, 2H), 7.40-7.61 (m, 4H), 6.38 (d, J = 8.0 Hz, 1H), 5.20 (s, 2H), 4.08-4.16 (m, 1H), 3.93 (s, 3H), 3.85 (s, 3H), 2.68-2.81 (m, 1H), 2.55-2.62 (m, 1H), 2.28- 2.38 (m, 1H), 2.07-2.17 (m, 1H). LCMS: calc. for C₂₄H₂₂N₄O₅: 446.2, found: [M + H]⁺ 447.1 |
| 47 | 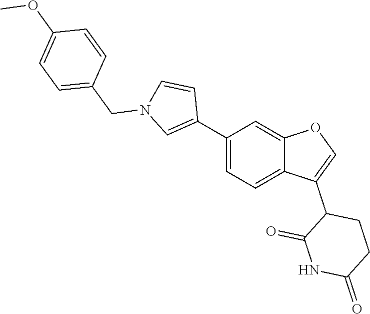 [SMILES: COc1ccc(Cn2ccc(-c3ccc4c(C5CCC(=O)NC5=O)coc4c3)c2)cc1] | ¹H NMR (400 MHz, DMSO-d₆) δ 10.88 (s, 1H), 8.43 (s, 0.08H, FA), 7.79 (s, 1H), 7.64 (s, 1H), 7.45-7.49 (m, 1H), 7.38-7.42 (m, 1H), 7.34 (s, 1H), 7.24 (d, J = 8.4 Hz, 2H), 6.91 (d, J = 8.4 Hz, 2H), 6.85 (d, J = 2.0 Hz, 1H), 6.49 (s, 1H), 5.02 (s, 2H), 4.08-4.12 (m, 1H), 3.73 (s, 3H), 2.69- 2.79 (m, 1H), 2.53-2.57 (m, 1H), 2.30-2.37 (m, 1H), 2.09-2.16 (m, 1H). LCMS: calc. for C₂₅H₂₂N₂O₄: 414.2, found: [M + H]⁺ 415.0 |
| 48 | 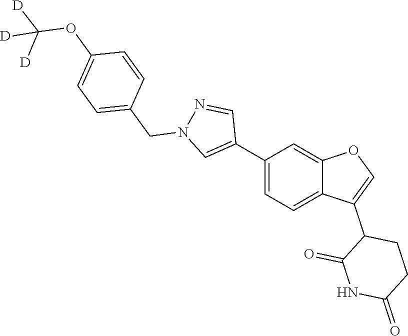 [SMILES: [2H]C([2H])([2H])Oc1ccc(Cn2cc(-c3ccc4c(C5CCC(=O)NC5=O)coc4c3)cn2)cc1] | ¹H NMR (400 MHz, DMSO-d₆) δ 10.90 (s, 1H), 8.29 (s, 1H), 7.96 (s, 1H), 7.85 (s, 1H), 7.78 (s, 1H), 7.57-7.44 (m, 2H), 7.26 (d, J = 8.4 Hz, 2H), 6.91 (d, J = 8.4 Hz, 2H), 5.26 (s, 2H), 4.12 (dd, J = 12.4, 4.8 Hz, 1H), 2.80-2.67 (m, 1H), 2.62-2.53 (m, 1H), 2.33-2.30 (m, 1H), 2.17- 2.02 (m, 1H). LCMS: calc. for C₂₄H₁₈D₃N₃O₄: 418.2, found: [M + H]⁺ 419.4 |
| 49 | 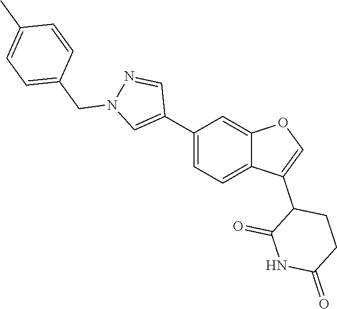 [SMILES: Cc1ccc(Cn2cc(-c3ccc4c(C5CCC(=O)NC5=O)coc4c3)cn2)cc1] | ¹H NMR (400 MHz, DMSO-d₆) δ 10.88 (s, 1H), 8.30 (s, 1H), 7.97 (s, 1H), 7.85 (s, 1H), 7.78 (s, 1H), 7.52-7.57 (m, 1H), 7.45-7.50 (m, 1H), 7.18 (d, J = 6.4 Hz, 4H), 5.29 (s, 2H), 4.12 (dd, J = 12.0, 4.8 Hz, 1H), 2.69-2.79 (m, 1H), 2.56-2.62 (m, 1H), 2.30-2.35 (m, 1H), 2.28 (s, 3H), 2.08- 2.17 (m, 1H). LCMS: calc. for C₂₄H₂₁N₃O₃: 399.2, found: [M + H]⁺ 400.2 |
| 50 | 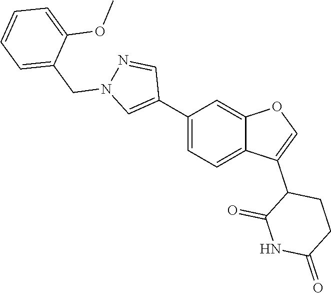 [SMILES: COc1ccccc1Cn1cc(-c2ccc3c(C4CCC(=O)NC4=O)coc3c2)cn1] | ¹H NMR (400 MHz, DMSO-d₆) δ 10.91 (s, 1H), 8.24 (s, 1H), 7.98 (s, 1H), 7.85 (s, 1H), 7.80 (s, 1H), 7.56-7.43 (m, 2H), 7.34-7.25 (m, 1H), 7.04 (d, J = 8.4 Hz, 1H), 6.99-6.85 (m, 2 H), 5.30 (s, 2 H), 4.12 (dd, J = 12.4, 4.8 Hz, 1H), 3.84 (s, 3H), 2.80-2.68 (m, 1H), 2.63-2.54 (m, 1H), 2.32 (q, J = 12.4, 4.4 Hz, 1H), 2.19-2.04 (m, 1H). LCMS: calc. for C₂₄H₂₁N₃O₄: 415.1, found: [M + H]⁺ 416.4 |
| 51 | 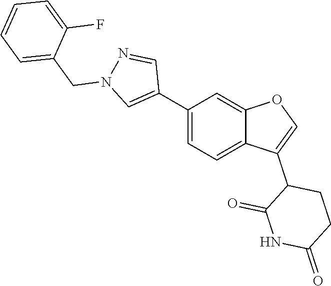 [SMILES: O=C1CCC(c2coc3cc(-c4cnn(Cc5ccccc5F)c4)ccc23)C(=O)N1] | ¹H NMR (400 MHz, DMSO-d₆) δ 10.89 (br s, 1H), 8.32 (s, 1H), 7.99 (s, 1H), 7.87-7.77 (m, 2H), 7.57-7.46 (m, 2H), 7.42-7.33 (m, 1H), 7.29-7.13 (m, 3H), 5.40 (s, 2H), 4.14-4.10 (m, 1H), 2.80-2.68 (m, 1H), 2.60-2.59 (m, 1H), 2.33-2.30 (m, 1H), 2.14-2.11 (m, 1H). ¹⁹F NMR (376 MHz, DMSO-d₆) δ −118.40. LCMS: calc. for C₂₃H₁₈FN₃O₃: 403.1, found: [M + H]⁺ 404.2 |
| 52 | 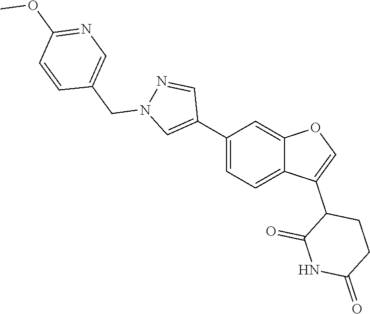 [SMILES: COc1ccc(Cn2cc(-c3ccc4c(C5CCC(=O)NC5=O)coc4c3)cn2)cn1] | ¹H NMR (400 MHz, DMSO-d₆) δ 10.72-11.04 (m, 1H), 8.33 (s, 1H), 8.21 (d, J = 1.6 Hz, 1H), 7.98 (s, 1H), 7.86 (s, 1H), 7.79 (s, 1H), 7.68 (dd, J = 8.4, 2.4 Hz, 1H), 7.52-7.59 (m, 1H), 7.44- 7.51 (m, 1H), 6.82 (d, J = 8.4 Hz, 1H), 5.30 (s, 2H), 4.13-4.20 (m, 1H), 3.84 (s, 3H), 2.70-2.79 (m, 1H), 2.55-2.60 (m, 1H), 2.29-2.37 (m, 1H) 2.07-2.20 (m, 1H). LCMS: calc. for C₂₃H₂₀N₄O₄: 416.1, found: [M + H]⁺ 417.1 |
| 53 | 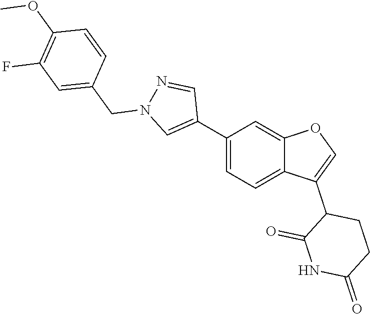 [SMILES: COc1ccc(Cn2cc(-c3ccc4c(C5CCC(=O)NC5=O)coc4c3)cn2)cc1F] | ¹H NMR (400 MHz, DMSO-d₆) δ 10.90 (s, 1H), 8.42 (s, 0.11H, FA), 8.32 (s, 1H), 7.98 (s, 1H), 7.86 (s, 1H), 7.79 (s, 1H), 7.55 (d, J = 8.4 Hz, 1H), 7.47 (d, J = 8.4 Hz, 1H), 7.07-7.22 (m, 3H), 5.27 (s, 2H), 4.09-4.16 (s, 1H), 3.81 (m, 3H), 2.69-2.80 (m, 1H), 2.53-2.62 (m, 1H), 2.26-2.42 (m, 1H), 2.08-2.17 (m, 1H). LCMS: calc. for C₂₄H₂₀FN₃O₄: 433.1, found: [M + H]⁺ 433.9. |
| 54 | 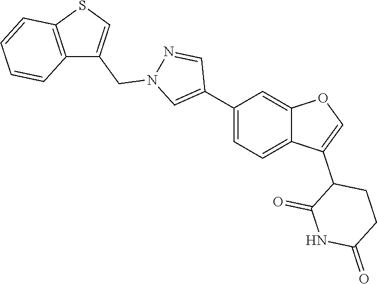 [SMILES: O=C1CCC(c2coc3cc(-c4cnn(Cc5csc6ccccc56)c4)ccc23)C(=O)N1] | ¹H NMR (400 MHz, DMSO-d₆) δ 10.89 (s, 1H), 8.39 (s, 1H), 7.91-8.05 (m, 3H), 7.80-7.89 (m, 1H), 7.79 (s, 1H), 7.74 (s, 1H), 7.51-7.57 (m, 1H), 7.45-7.50 (m, 1H), 7.36-7.44 (m, 2H), 5.62 (s, 2H), 4.08-4.17 (m, 1H), 2.68-2.80 (m, 1H), 2.54-2.62 (m, 1H), 2.26-2.35 (m, 1H), 2.07-2.17 (m, 1H). LCMS: calc. for C₂₅H₁₉N₃O₃S: 441.1, found: [M + H]⁺ 441.8 |
| 55 | 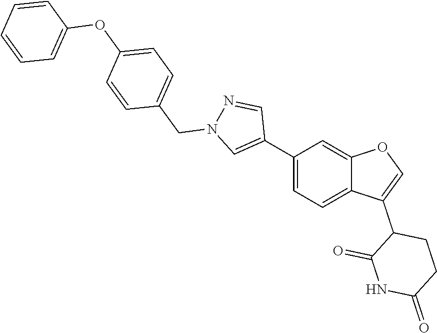 [SMILES: O=C1CCC(c2coc3cc(-c4cnn(Cc5ccc(Oc6ccccc6)cc5)c4)ccc23)C(=O)N1] | ¹H NMR (400 MHz, DMSO-d₆) δ 10.90 (s, 1H), 8.35 (s, 1H), 8.00 (s, 1H), 7.86 (s, 1H), 7.80 (s, 1H), 7.56 (d, J = 8.4 Hz, 1H), 7.49 (dd, J = 8.4 Hz, 1.2 Hz, 1H), 7.30-7.42 (m, 4H), 7.14 (t, J = 7.2 Hz, 1H), 6.96-7.03 (m, 4H), 5.33 (s, 2H), 4.10-4.17 (m, 1H), 2.70-2.78 (m, 1H), 2.56-2.62 (m, 1H), 2.32-2.36 (m, 1H), 2.10-2.17 (m, 1H). LCMS: calc. for C₂₉H₂₃N₃O₄: 477.2, found: [M + H]⁺ 478.1 |
| 56 | 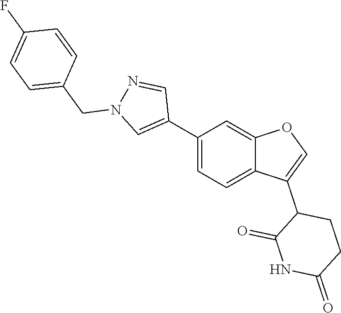 [SMILES: O=C1CCC(c2coc3cc(-c4cnn(Cc5ccc(F)cc5)c4)ccc23)C(=O)N1] | ¹H NMR (400 MHz, DMSO-d₆) δ 10.89 (s, 1H), 8.36-8.31 (m, 1H), 7.98 (s, 1H), 7.88-7.82 (m, 1H), 7.79 (s, 1H), 7.57-7.51 (m, 1H), 7.51-7.45 (m, 1H), 7.35 (dd, J = 5.6, 8.8 Hz, 2H), 7.24- 7.14 (m, 2H), 5.33 (s, 2H), 4.12 (dd, J = 4.8, 12.0 Hz, 1H), 2.80-2.68 (m, 1H), 2.60-2.50 (m, 1H), 2.33-2.31 (m, 1H), 2.17-2.08 (m, 1H). LCMS: calc. for C₂₃H₁₈FN₃O₃: 403.1, found: [M + H]⁺ 404.2 |
| 57 | 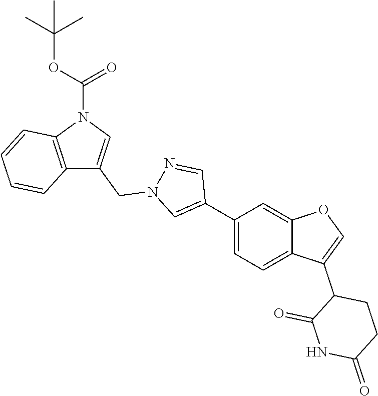 [SMILES: CC(C)(C)OC(=O)n1cc(Cn2cc(-c3ccc4c(C5CCC(=O)NC5=O)coc4c3)cn2)c2ccccc21] | ¹H NMR (400 MHz, DMSO-d₆) δ 10.89 (s, 1H), 8.34 (s, 1H), 8.05 (d, J = 8.4 Hz, 1H), 7.97 (s, 1H), 7.83 (d, J = 11.6 Hz, 2H), 7.77 (d, J = 0.8 Hz, 1H), 7.63 (d, J = 8.0 Hz, 1H), 7.51-7.54 (m, 1H), 7.43-7.48 (m, 1H), 7.31-7.35 (m, 1H), 7.23 (t, J = 7.2 Hz, 1H), 5.49 (s, 2H), 4.09-4.13 (m, 1H), 2.69-2.73 (m, 1H), 2.58-2.60 (m, 1H), 2.28- 2.31 (m, 1H), 2.09-2.14 (m, 1H), 1.64 (s, 9H). LCMS: calc. for C₃₀H₂₈N₄O₆: 524.2, found: [M + H]⁺ 525.1 |
| 58 | 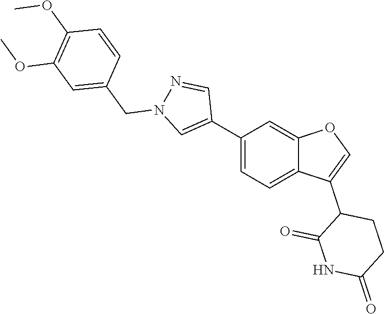 [SMILES: COc1ccc(Cn2cc(-c3ccc4c(C5CCC(=O)NC5=O)coc4c3)cn2)cc1OC] | ¹H NMR (400 MHz, DMSO-d₆) δ 10.89 (s, 1H), 8.29 (s, 1H), 7.97 (s, 1H), 7.85 (s, 1H), 7.79 (s, 1H), 7.52-7.56 (m, 1H), 7.45-7.50 (m, 1H), 6.98 (d, J = 1.6 Hz, 1H), 6.90-6.95 (m, 1H), 6.84 (dd, J = 8.4, 2.0 Hz, 1H), 5.25 (s, 2H), 4.12 (dd, J = 12.0, 4.8 Hz, 1H), 3.74 (s, 3H), 3.73 (s, 3H), 2.69-2.79 (m, 1H), 2.54-2.61 (m, 1H), 2.21-2.38 (m, 1H), 2.09-2.16 (m, 1H). LCMS: calc. for C₂₅H₂₃N₃O₅: 445.2, found: [M + H]⁺ 446.0 |
| 59 | 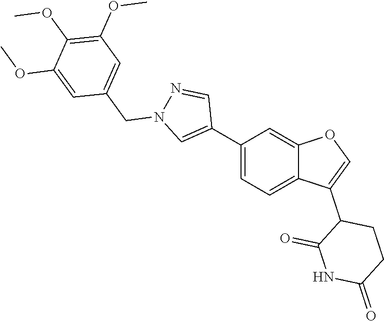 [SMILES: COc1cc(Cn2cc(-c3ccc4c(C5CCC(=O)NC5=O)coc4c3)cn2)cc(OC)c1OC] | ¹H NMR (400 MHz, DMSO-d₆) δ 10.90 (s, 1H), 8.34 (s, 1H), 7.99 (s, 1H), 7.78-7.88 (m, 2H), 7.48-7.57 (m, 2H), 6.67 (s, 2H), 5.26 (s, 2H), 4.10-4.16 (m, 1H), 3.75 (s, 6H), 3.64 (s, 3H), 2.70-3.76 (m, 1H), 2.59-2.62 (m, 1H), 2.36-3.41 (m, 1H), 2.10-2.16 (m, 1H). LCMS: calc. for C₂₆H₂₅N₃O₆: 475.2, found: [M + H]⁺ 476.0 |
| 60 | 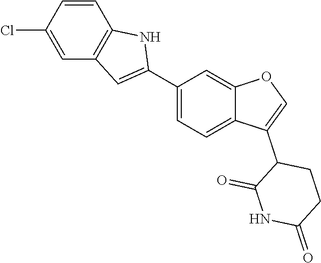 [SMILES: O=C1CCC(c2coc3cc(-c4cc5cc(Cl)ccc5[nH]4)ccc23)C(=O)N1] | ¹H NMR (400 MHz, DMSO-d₆) δ 11.79 (s, 1H), 10.92 (s, 1H), 8.07 (s, 1H), 7.96 (s, 1H), 7.75- 7.81 (m, 1H), 7.67 (d, J = 8.0 Hz, 1H), 7.56 (d, J = 1.6 Hz, 1H), 7.41 (d, J = 8.4 Hz, 1H), 7.09 (dd, J = 8.4, 2.0 Hz, 1H), 6.96 (s, 1H), 4.17 (dd, J = 12.0, 4.8 Hz, 1H), 2.71-2.82 (m, 1H), 2.56-2.64 (m, 1H), 2.30-2.42 (m, 1H), 2.10-2.19 (m, 1H). LCMS: calc. for C₂₁H₁₅ClN₂O₃: 378.1, found: [M + H]⁺ 379.2. |
| 61 | 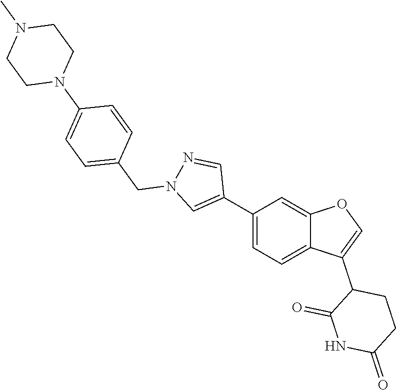 [SMILES: CN1CCN(c2ccc(Cn3cc(-c4ccc5c(C6CCC(=O)NC6=O)coc5c4)cn3)cc2)CC1] | ¹H NMR (400 MHz, DMSO-d₆) δ 10.90 (s, 1H), 8.27 (s, 1H), 7.95 (s, 1H), 7.85 (s, 1H), 7.78 (s, 1H), 7.56-7.51 (m, 1H), 7.50-7.43 (m, 1H), 7.18 (d, J = 8.0 Hz, 2H), 6.90 (d, J = 8.0 Hz, 2H), 5.20 (s, 2H), 4.12 (dd, J = 4.8, 12.0 Hz, 1H), 3.14- 3.05 (m, 4H), 2.81-2.66 (m, 1H), 2.63-2.53 (m, 1H), 2.45-2.39 (m, 4H), 2.33-2.31 (m, 1H), 2.22-2.18 (m, 3H), 2.16-2.05 (m, 1H). LCMS: calc. for C₂₈H₂₉N₅O₃: 483.2, found: [M + H]⁺ 484.4. |
| 62 | 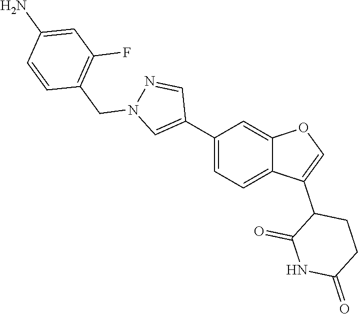 [SMILES: Nc1ccc(Cn2cc(-c3ccc4c(C5CCC(=O)NC5=O)coc4c3)cn2)c(F)c1] | ¹H NMR (400 MHz, DMSO-d₆) δ 10.89 (s, 1H), 8.28 (s, 0.15H, FA), 8.17 (s, 1H), 7.93 (s, 1H), 7.85 (s, 1H), 7.78 (s, 1H), 7.50-7.57 (m, 1H), 7.44-7.48 (m, 1H), 6.97-7.07 (m, 1H), 6.28-6.38 (m, 2H), 5.47 (s, 2H), 5.15 (s, 2H), 4.08-4.18 (m, 1H), 2.69-2.77 (m, 1H), 2.54-2.63 (m, 1H), 2.27- 2.40 (m, 1H), 2.07-2.18 (m, 1H) LCMS: calc. for C₂₃H₁₉FN₄O₃: 418.1, found: [M + H]⁺ 419.1 |
| 63 | 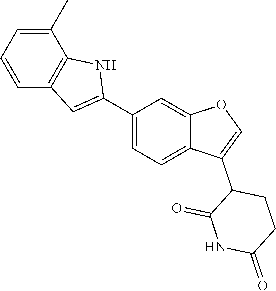 [SMILES: Cc1cccc2cc(-c3ccc4c(C5CCC(=O)NC5=O)coc4c3)[nH]c12] | ¹H NMR (400 MHz, DMSO-d₆) δ 11.08 (s, 1H), 10.93 (s, 1H), 8.20 (s, 1H), 7.95 (s, 1H), 7.87- 7.81 (m, 1H), 7.65 (d, J = 8.4 Hz, 1H), 7.35 (d, J = 6.8 Hz, 1H), 6.96 (d, J = 2.0 Hz, 1H), 6.94- 6.85 (m, 2H), 4.21-4.12 (m, 1H), 2.75 (br dd, J = 5.6, 12.4 Hz, 1H), 2.62 (br t, J = 4.0 Hz, 1H), 2.56 (s, 3H), 2.43-2.31 (m, 1H), 2.21-2.11 (m, 1H) LCMS: calc. for C₂₂H₁₈N₂O₃: 358.1, found: [M + H]⁺ 359.4. |
| 64 | 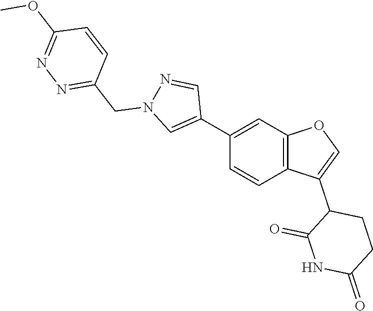 [SMILES: COc1ccc(Cn2cc(-c3ccc4c(C5CCC(=O)NC5=O)coc4c3)cn2)nn1] | ¹H NMR (400 MHz, DMSO-d₆) δ 10.90 (s, 1H), 8.39 (s, 1H), 8.02 (s, 1H), 7.86 (s, 1H), 7.81 (s, 1H), 7.54-7.58 (m, 1H), 7.47-7.52 (m, 2H), 7.23 (d, J = 9.2 Hz, 1H), 5.58 (s, 2H), 4.13 (dd, J = 11.6, 4.8 Hz, 1H), 4.02 (s, 3H), 2.73 (m, 1H), 2.62-2.69 (m, 1H), 2.33 (m, 1H), 2.09-2.17 (m, 1H). LCMS: calc. for C₂₂H₁₉N₅O₄: 417.1, found: [M + H]⁺ 417.9. |
| 65 | 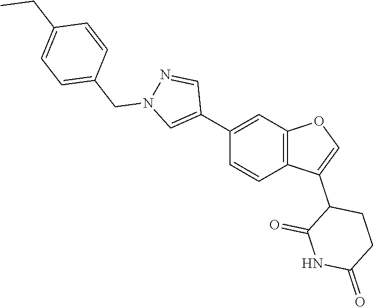 [SMILES: CCc1ccc(Cn2cc(-c3ccc4c(C5CCC(=O)NC5=O)coc4c3)cn2)cc1] | ¹H NMR (400 MHz, DMSO-d₆) δ 10.80 (s, 1H), 8.30 (s, 1H), 7.99-7.96 (m, 1H), 7.85 (s, 1H), 7.79 (s, 1H), 7.56-7.52 (m, 1H), 7.49-7.46 (m, 1H), 7.23-7.17 (m, 4H), 5.29 (s, 2H), 4.12 (dd, J = 4.8, 12.0 Hz, 1H), 2.61-2.57 (m, 2H), 2.57- 2.54 (m, 2H), 2.36-2.29 (m, 1H), 2.16-2.08 (m, 1H), 2.17-2.08 (m, 1H), 1.15 (t, J = 7.6 Hz, 3H). LCMS: calc. for C₂₅H₂₃N₃O₃: 413.1, found: [M + H]⁺ 414.2. |
| 66 | 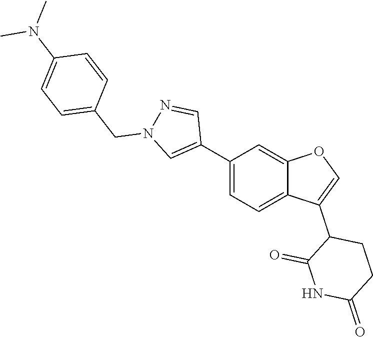 [SMILES: CN(C)c1ccc(Cn2cc(-c3ccc4c(C5CCC(=O)NC5=O)coc4c3)cn2)cc1] | ¹H NMR (400 MHz, DMSO-d₆) δ 10.91 (s, 1 H), 8.38 (s, 1 H), 8.00 (s, 1 H), 7.86 (s, 1 H), 7.79 (d, J = 4.0 Hz, 1 H), 7.68 (m, 2 H), 7.58-7.52 (m, 1 H), 7.49 (d, J = 12.0 Hz, 1 H), 7.47-7.45 (m, 1 H), 7.43 (s, 1 H), 5.38 (s, 2 H), 4.13 (m, 1 H), 3.07 (s, 6 H), 2.75 (m, 1 H), 2.58 (m, 1 H), 2.32 (m, 1 H), 2.17-2.04 (m, 1 H). LCMS: calc. for C₂₅H₂₄N₄O₃: 428.2, found: [M + H]⁺ 429.1. |
| 67 | 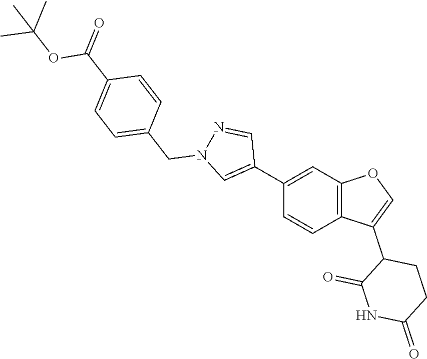 [SMILES: CC(C)(C)OC(=O)c1ccc(Cn2cc(-c3ccc4c(C5CCC(=O)NC5=O)coc4c3)cn2)cc1] | ¹H NMR (400 MHz, DMSO-d₆) δ 10.89 (s, 1 H), 8.35 (s, 1 H), 8.02 (s, 1 H), 7.89 (s, 1 H), 7.87 (d, J = 8.0 Hz, 1 H), 7.80 (s, 1 H), 7.58-7.52 (m, 1 H), 7.52-7.46 (m, 2 H), 5.44 (s, 2 H), 4.13 (dd, J = 12.0, 4.8 Hz, 1 H), 2.79-2.69 (m, 1 H), 2.61- 2.53 (m, 1 H), 2.38-2.26 (m, 1 H), 2.13 (m, 1 H), 1.53 (s, 9 H). LCMS: calc. for C₂₈H₂₇N₃O₅: 485.1, found: [M + H]⁺ 486.1. |
| 68 | 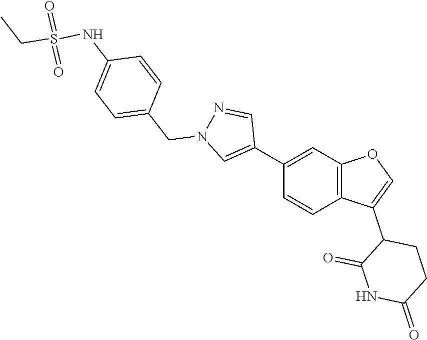 [SMILES: CCS(=O)(=O)Nc1ccc(Cn2cc(-c3ccc4c(C5CCC(=O)NC5=O)coc4c3)cn2)cc1] | ¹H NMR (400 MHz, DMSO-d₆) δ 10.90 (s, 1H), 9.81 (br s, 1H), 8.32 (s, 1H), 7.98 (s, 1H), 7.86 (s, 1H), 7.79 (s, 1H), 7.57-7.43 (m, 2H), 7.31-7.14 (m, 4H), 5.28 (s, 2H), 4.12 (dd, J = 12.0, 4.8 Hz, 1H), 3.06 (q, J = 7.2 Hz, 2H), 2.83-2.65 (m, 1H), 2.58 (td, J = 12.0, 3.6 Hz, 1H), 2.40-2.26 (m, 1H), 2.19-2.07 (m, 1H), 1.16 (t, J = 7.2 Hz, 3H). LCMS: calc. for C₂₅H₂₄N₄O₅S: 492.1, found: [M + H]⁺ 493.3. |
| 69 | 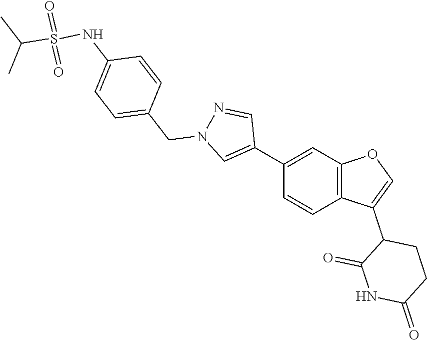 [SMILES: CC(C)S(=O)(=O)Nc1ccc(Cn2cc(-c3ccc4c(C5CCC(=O)NC5=O)coc4c3)cn2)cc1] | ¹H NMR (400 MHz, DMSO-d₆) δ 10.89 (s, 1H), 9.91-9.58 (m, 1H), 8.31 (s, 1H), 7.97 (s, 1H), 7.85 (s, 1H), 7.78 (s, 1H), 7.57-7.44 (m, 2H), 7.31-7.14 (m, 4H), 5.27 (s, 2H), 4.12 (dd, J = 4.8, 12.0 Hz, 1H), 3.19 (td, J = 8.0, 12.0 Hz, 1H), 2.81-2.70 (m, 1H), 2.59 (d, J = 4.0 Hz, 1H), 2.37-2.25 (m, 1H), 2.18-2.02 (m, 1H), 1.21 (d, J = 8.0 Hz, 6H). LCMS: calc. for C₂₆H₂₆N₄O₅S: 506.1, found: [M + H]⁺ 507.2. |
| 70 | 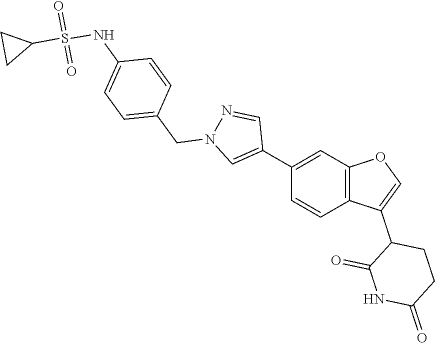 [SMILES: O=C1CCC(c2coc3cc(-c4cnn(Cc5ccc(NS(=O)(=O)C6CC6)cc5)c4)ccc23)C(=O)N1] | ¹H NMR (400 MHz, DMSO-d₆) δ 10.90 (br s, 1H), 9.70 (br d, J = 8.0 Hz, 1H), 8.32 (s, 1H), 7.98 (s, 1H), 7.85 (s, 1H), 7.79 (s, 1H), 7.56- 7.52 (m, 1H), 7.50-7.45 (m, 1H), 7.31-7.24 (m, 2H), 7.21 (d, J = 8.8 Hz, 2H), 5.29 (s, 2H), 4.12 (dd, J = 4.8, 12.0 Hz, 1H), 2.82-2.68 (m, 1H), 2.64-2.53 (m, 3H), 2.39-2.23 (m, 1H), 2.18- 2.01 (m, 1H), 0.95-0.86 (m, 4H). LCMS: calc. for C₂₆H₂₄N₄O₅S: 504.1, found: [M + H]⁺ 505.1. |
| 141 | 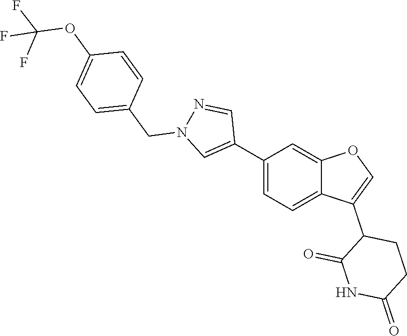 [SMILES: O=C1CCC(c2coc3cc(-c4cnn(Cc5ccc(OC(F)(F)F)cc5)c4)ccc23)C(=O)N1] | ¹H NMR (400 MHz, DMSO-d₆) δ 10.90 (s, 1H), 8.37 (s, 1H), 8.01 (s, 1H), 7.77-7.90 (m, 2H), 7.32-7.59 (m, 6H), 5.40 (s, 2H), 4.13 (dd, J = 11.6, 4.0 Hz, 1H), 2.69-2.78 (m, 1H), 2.58-2.62 (m, 1H), 2.32 (d, J = 6.4 Hz, 1H), 2.12 (dd, J = 8.4, 4.0 Hz, 1H). LCMS: calc. for C₂₄H₁₈F₃N₃O₄: 469.1, found: [M + H]⁺ 470.2 |
| 142 | 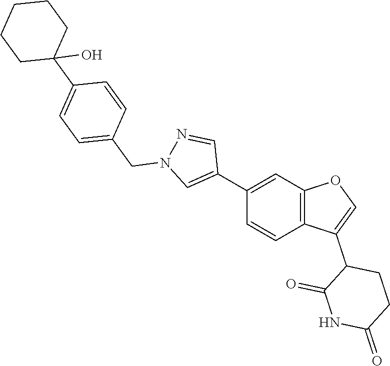 [SMILES: O=C1CCC(c2coc3cc(-c4cnn(Cc5ccc(C6(O)CCCCC6)cc5)c4)ccc23)C(=O)N1] | ¹H NMR (400 MHz, DMSO-d₆) δ 10.90 (s, 1H), 8.34 (s, 1H), 7.98 (s, 1H), 7.86 (s, 1H), 7.80 (s, 1H), 7.55 (d, J = 8.4 Hz, 1H), 7.43-7.51 (m, 3H), 7.23 (d, J = 8.4 Hz, 2H), 5.31 (s, 2H), 4.66 (s, 1H), 4.05-4.20 (m, 1H), 2.70-2.80 (m, 1H), 2.55- 2.62 (m, 1H), 2.31-2.35 (m, 1H), 2.10-2.16 (m, 1H), 1.45-1.74 (m, 10H). LCMS: calc. for C₂₉H₂₉N₃O₄: 483.2, found: [M + H]⁺ 484.3 |
| 143 | 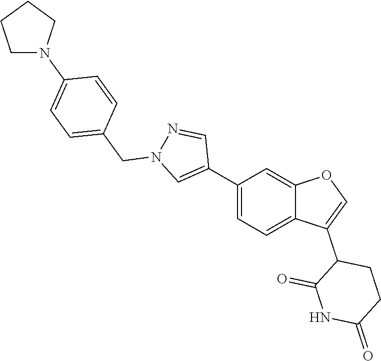 [SMILES: O=C1CCC(c2coc3cc(-c4cnn(Cc5ccc(N6CCCC6)cc5)c4)ccc23)C(=O)N1] | ¹HNMR (400 MHz, DMSO-d₆) δ 10.88 (br s, 1H), 8.21 (s, 1H), 7.92 (s, 1H), 7.84 (s, 1H), 7.76 (d, J = 1.2 Hz, 1H), 7.55-7.51 (m, 1H), 7.48- 7.43 (m, 1H), 7.17 (d, J = 8.8 Hz, 2H), 6.50 (d, J = 8.4 Hz, 2H), 5.16 (s, 2H), 4.12 (dd, J = 12.4, 4.4 Hz, 1H), 3.22-3.15 (m, 4H), 2.79-2.66 (m, 1H), 2.57 (m, 1H), 2.38-2.26 (m, 1H), 2.12 (m, 1H), 1.97-1.89 (m, 4H). MS: (ESI) m/z calculated for C₂₇H₂₆N₄O₃ [M + H]⁺ 455.2, found 455.1. |
| 144 | 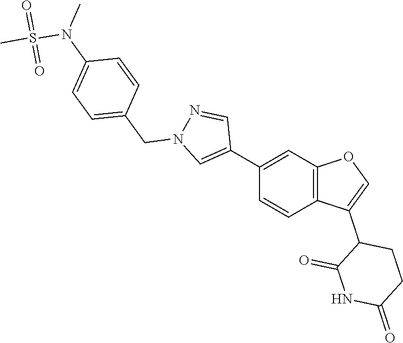 [SMILES: CN(c1ccc(Cn2cc(-c3ccc4c(C5CCC(=O)NC5=O)coc4c3)cn2)cc1)S(C)(=O)=O] | ¹HNMR (400 MHz, DMSO-d₆) δ 10.96-10.84 (m, 1H), 8.37 (s, 1H), 8.00 (s, 1H), 7.86 (s, 1H), 7.80 (s, 1H), 7.54 (s, 1H), 7.50-7.46 (m, 1H), 7.44-7.35 (m, 2H), 7.35-7.29 (m, 2H), 5.35 (s, 2H), 4.17-4.06 (m, 1H), 3.22-3.19 (m, 3H), 2.93-2.90 (m, 3H), 2.79-2.65 (m, 1H), 2.63- 2.54 (m, 1H), 2.39-2.28 (m, 1H), 2.19-2.08 (m, 1H). MS: (ESI) m/z calculated for C₂₅H₂₄N₄O₅S [M + H]⁺ 493.1, found 493.1. |
| 145 | 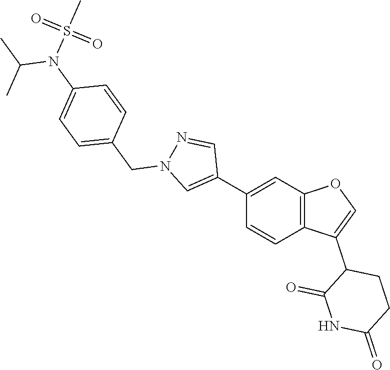 [SMILES: CC(C)N(c1ccc(Cn2cc(-c3ccc4c(C5CCC(=O)NC5=O)coc4c3)cn2)cc1)S(C)(=O)=O] | ¹H NMR (400 MHz, DMSO-d₆) δ 10.89 (s, 1H), 8.40 (s, 1H), 8.02 (s, 1H), 7.86 (s, 1H), 7.81 (s, 1H), 7.59-7.47 (m, 2H), 7.35-7.23 (m, 4H), 5.40 (s, 2H), 4.31 (quin, J = 6.8 Hz, 1H), 4.13 (dd, J = 12.0,12.0 Hz, 1H), 3.04 (s, 3H), 2.81- 2.68 (m, 1H), 2.58 (td, J = 16.0, 3.2 Hz, 1H), 2.41- 2.25 (m, 1H), 2.13 (dt, J = 8.4, 4.4 Hz, 1H), 1.05 (d, J = 6.0 Hz, 6H). MS: (ESI) m/z calculated for C₂₇H₂₈N₄O₅S [M + H]⁺ 521.1, found 521.4. |
| 146 | 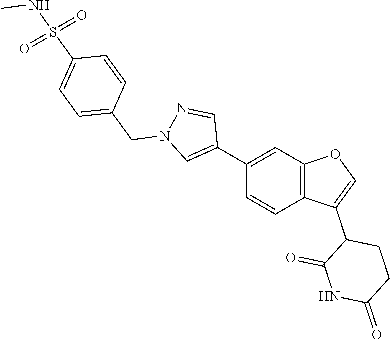 [SMILES: CNS(=O)(=O)c1ccc(Cn2cc(-c3ccc4c(C5CCC(=O)NC5=O)coc4c3)cn2)cc1] | ¹H NMR (400 MHz, DMSO-d₆) δ 10.89-10.88 (m, 1H), 8.40-8.38 (m, 1H), 8.04-8.01 (m, 1H), 7.86 (s, 1H), 7.81-7.79 (m, 1H), 7.78-7.76 (m, 1H), 7.76-7.73 (m, 1H), 7.54 (s, 1H), 7.51-7.41 (m, 4H), 5.46 (s, 2H), 4.16-4.09 (m, 1H), 2.79- 2.69 (m, 1H), 2.62-2.55 (m, 1H), 2.39 (d, J = 4.8 Hz, 3H), 2.34-2.26 (m, 1H), 2.17-2.09 (m, 1H), 2.06 (s, 1H). MS: (ESI) m/z calculated for C₂₄H₂₂N₄O₅S [M + H]⁺ 479.1, found 479.0. |
| 147 | 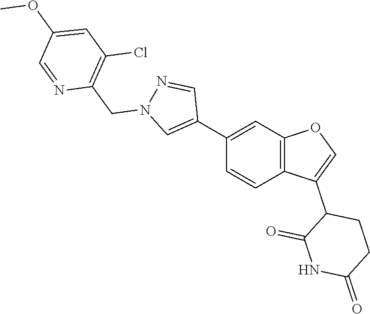 [SMILES: COc1cnc(Cn2cc(-c3ccc4c(C5CCC(=O)NC5=O)coc4c3)cn2)c(Cl)c1] | ¹H NMR (400 MHz, DMSO-d₆) δ 11.01-10.72 (m, 1H), 8.30-8.22 (m, 2H), 7.97-7.90 (m, 1H), 7.87-7.79 (m, 2H), 7.67-7.62 (m, 1H), 7.56- 7.46 (m, 2H), 5.54-5.44 (m, 2H), 4.17-4.08 (m, 1H), 3.90-3.81 (m, 3H), 2.78-2.69 (m, 1H), 2.58 (td, J = 4.0, 16.0 Hz, 1H), 2.37-2.29 (m, 1H), 2.17-2.08 (m, 1H). MS: (ESI) m/z calculated for C₂₃H₁₉ClN₄O₄ [M + H]⁺ 451.1, found 451.1. |
| 148 | 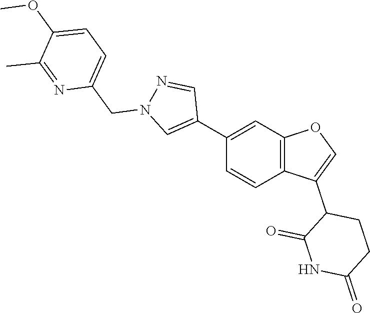 [SMILES: COc1ccc(Cn2cc(-c3ccc4c(C5CCC(=O)NC5=O)coc4c3)cn2)nc1C] | ¹H NMR (400 MHz, DMSO-d₆) δ 10.90-10.87 (m, 1H), 8.38-8.33 (m, 1H), 8.03-7.99 (m, 1H), 7.88-7.84 (m, 1H), 7.82-7.78 (m, 1H), 7.58- 7.53 (m, 2H), 7.51-7.46 (m, 1H), 7.24-7.16 (m, 1H), 5.44-5.41 (m, 2H), 4.13 (J = 4.8 Hz, 1H), 3.85-3.84 (m, 3H), 2.79-2.69 (m, 1H), 2.64- 2.54 (m, 2H), 2.45-2.42 (m, 3H), 2.36 (s, 1H), 2.18 (s, 1H). MS: (ESI) m/z calculated for C₂₄H₂₂N₄O₄ [M + H]⁺ 431.1, found 431.1. |
| 149 | 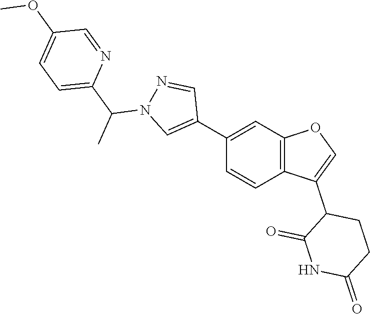 [SMILES: COc1ccc(C(C)n2cc(-c3ccc4c(C5CCC(=O)NC5=O)coc4c3)cn2)nc1] | ¹H NMR (400 MHz, DMSO-d6) δ 10.89 (s, 1 H), 8.38 (s, 1 H), 8.24 (d, J = 2.8 Hz, 1 H), 7.97 (s, 1 H), 7.85 (s, 1 H), 7.81 (s, 1 H), 7.55-7.48 (m, 2 H), 7.36 (dd, J = 8.4, 2.8 Hz, 1 H), 7.12 (d, J = 8.8 Hz, 1 H), 5.62 (q, J = 6.8 Hz, 1 H), 4.12 (dd, J = 11.6, 4.8 Hz, 1 H), 3.80 (s, 3 H), 2.80-2.69 (m, 1 H), 2.61-2.55 (m, 1 H), 2.37-2.27 (m, 1 H), 2.16-2.09 (m, 1 H), 1.85 (d, J = 6.4 Hz, 3 H). MS: (ESI) m/z calculated for C₂₄H₂₂N₄O₄ [M + H]⁺ 431.2, found 431.1. |
| 150 | 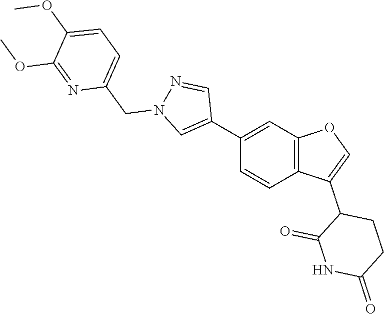 [SMILES: COc1ccc(Cn2cc(-c3ccc4c(C5CCC(=O)NC5=O)coc4c3)cn2)nc1OC] | ¹H NMR (400 MHz, DMSO-d₆) δ 11.06-10.72 (m, 1H), 8.32 (s, 1H), 8.02-7.97 (m, 1H), 7.88- 7.84 (m, 1H), 7.83-7.79 (m, 1H), 7.57-7.53 (m, 1H), 7.51-7.47 (m, 1H), 7.25 (d, J = 8.0 Hz, 1H), 6.71 (d, J = 8.0 Hz, 1H), 5.27 (s, 2H), 4.15- 4.11 (m, 1H), 3.85 (s, 3H), 3.75 (s, 3H), 2.81- 2.69 (m, 1H), 2.63-2.54 (m, 1H), 2.39-2.27 (m, 1H), 2.19-2.09 (m, 1H). MS: (ESI) m/z calculated for C₂₄H₂₂N₄O₅ [M + H]⁺ 447.1, found 447.0. |
| 151 | 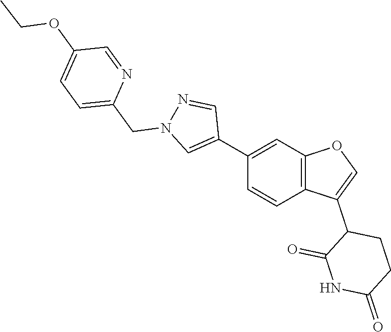 [SMILES: CCOc1ccc(Cn2cc(-c3ccc4c(C5CCC(=O)NC5=O)coc4c3)cn2)nc1] | ¹H NMR (400 MHz, DMSO-d₆) δ 10.94-10.79 (m, 1H), 8.36-8.28 (m, 1H), 8.25-8.18 (m, 1H), 8.00-7.96 (m, 1H), 7.88-7.77 (m, 2H), 7.58- 7.46 (m, 2H), 7.39-7.34 (m, 1H), 7.19-7.13 (m, 1H), 5.39-5.32 (m, 2H), 4.15-4.04 (m, 3H), 2.79-2.69 (m, 1H), 2.62-2.56 (m, 1H), 2.35- 2.28 (m, 1H), 2.19-2.08 (m, 1H), 1.35-1.30 (m, 3H). MS: (ESI) m/z calculated for C₂₄H₂₂N₄O₄ [M + H]⁺ 431.1, found 431.1. |
| 152 | 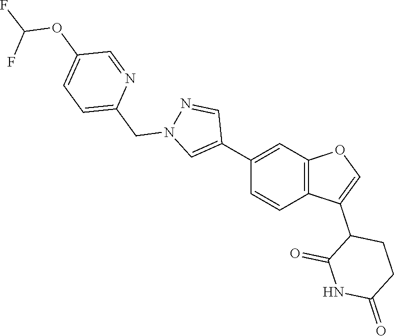 [SMILES: O=C1CCC(c2coc3cc(-c4cnn(Cc5ccc(OC(F)F)cn5)c4)ccc23)C(=O)N1] | ¹HNMR (400 MHz, DMSO-d₆) δ 10.89 (s, 1H), 8.46 (d, J = 2.8 Hz, 1H), 8.37 (s, 1H), 8.01 (s, 1H), 7.86 (s, 1H), 7.81 (s, 1H), 7.68 (dd, J = 2.8, 8.4 Hz, 1H), 7.58-7.47 (m, 2H), 7.31-7.10 (m, 2H), 5.46 (s, 2H), 4.13 (dd, J = 4.8, 11.6 Hz, 1H), 2.81-2.69 (m, 1H), 2.63-2.54 (m, 1H), 2.39- 2.27 (m, 1H), 2.14 (m, J = 1H). ¹⁹FNMR (376 MHz, DMSO-d₆) δ −82.485. MS: (ESI) m/z calculated for C₂₃H₁₈F₂N₄O₄[M + H]⁺ 453.1, found 453.4. |
| 153 | 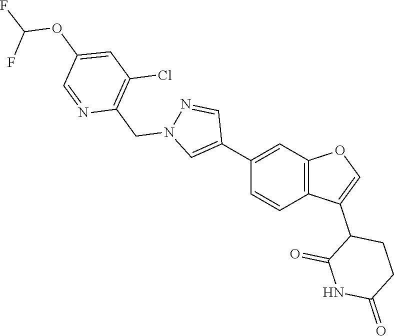 [SMILES: O=C1CCC(c2coc3cc(-c4cnn(Cc5ncc(OC(F)F)cc5Cl)c4)ccc23)C(=O)N1] | ¹H NMR (400 MHz, DMSO-d₆) δ 10.95-10.87 (m, 1H), 8.48-8.44 (m, 1H), 8.31 (s, 1H), 8.01 (d, J = 2.4 Hz, 1H), 7.96 (s, 1H), 7.88-7.84 (m, 1H), 7.81 (s, 1H), 7.57-7.19 (m, 3H), 5.62-5.54 (m, 2H), 4.18-4.08 (m, 1H), 2.75-2.66 (m, 1H), 2.62-2.54 (m, 1H), 2.37-2.30 (m, 1H), 2.12 (br dd, J = 4.8, 8.4 Hz, 1H). ¹⁹F NMR (376 MHz, DMSO-d₆) δ −83.25. MS: (ESI) m/z calculated for C₂₃H₁₇ClF₂N₄O₄ [M + H]⁺ 487.1, found 487.1. |
| 154 | 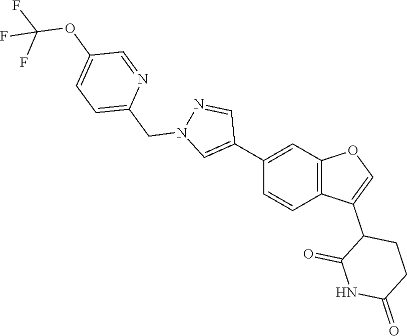 [SMILES: O=C1CCC(c2coc3cc(-c4cnn(Cc5ccc(OC(F)(F)F)cn5)c4)ccc23)C(=O)N1] | ¹H NMR (400 MHz, DMSO-d₆) δ 10.90 (s, 1H), 8.65 (d, J = 2.4 Hz, 1H), 8.40 (s, 1H), 8.04 (s, 1H), 7.91 (dd, J = 8.4, 1.6 Hz, 1H), 7.86 (s, 1H), 7.82 (s, 1H), 7.59-7.48 (m, 2H), 7.30 (d, J = 8.4 Hz, 1H), 5.52 (s, 2H), 4.13 (dd, J = 12.4, 4.8 Hz, 1H), 2.82-2.68 (m, 1H), 2.63-2.52 (m, 1H), 2.33 (dq, J = 12.4, 4.0 Hz, 1H), 2.21-2.04 (m, 1H). ¹⁹F NMR (400 MHz, DMSO-d₆) δ −57.25. MS: (ESI) m/z calculated for C₂₃H₁₇F₃N₄O₄ [M + H]⁺ 471.1, found 471.2. |
| 155 | 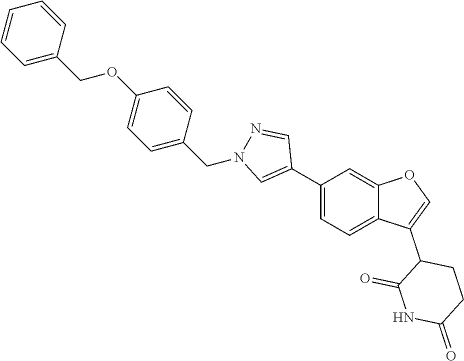 [SMILES: O=C1CCC(c2coc3cc(-c4cnn(Cc5ccc(OCc6ccccc6)cc5)c4)ccc23)C(=O)N1] | ¹H NMR (400 MHz, DMSO-d₆) δ 10.90 (s, 1H), 8.40-8.12 (m, 2H), 7.98 (s, 1H), 7.86 (s, 1H), 7.80 (s, 1H), 7.58-7.52 (m, 1H), 7.51-7.42 (m, 4H), 7.42-7.30 (m, 3H), 7.17 (d, J = 8.4 Hz, 1H), 5.36 (s, 2H), 5.17 (s, 2H), 4.13 (dd, J = 4.8, 11.6 Hz, 1H), 2.83-2.66 (m, 1H), 2.64-2.55 (m, 1H), 2.38-2.27 (m, 1H), 2.18-2.08 (m, 1H). MS: (ESI) m/z calculated for C₂₉H₂₄N₄O₄ [M + H]⁺ 493.1, found 493.1. |
| 156 | 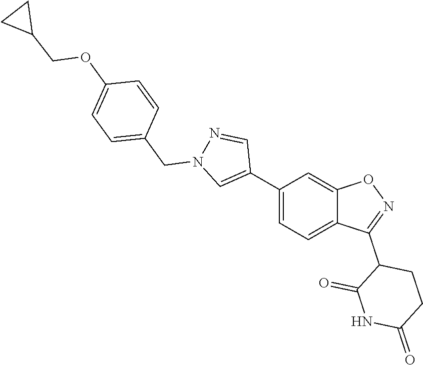 [SMILES: O=C1CCC(c2noc3cc(-c4cnn(Cc5ccc(OCC6CC6)cc5)c4)ccc23)C(=O)N1] | ¹H NMR (400 MHz, DMSO-d₆) δ 10.92-10.87 (m, 1H), 8.33 (s, 1H), 8.27 (s, 1H), 7.99 (s, 1H), 7.85 (s, 1H), 7.80 (s, 1H), 7.58-7.52 (m, 1H), 7.51-7.46 (m, 1H), 7.44-7.41 (m, 1H), 7.21 (d, J = 8.4 Hz, 1H), 5.38 (s, 2H), 4.14-4.10 (m, 1H), 3.89 (d, J = 7.2 Hz, 2H), 2.79-2.67 (m, 1H), 2.60-2.54 (m, 1H), 2.34-2.30(m, 1H), 2.18-2.09 (m, 1H), 1.22-1.16 (m, 1H), 0.61- 0.52 (m, 2H), 0.33-0.36 (m, 2H). MS: (ESI) m/z calculated for C₂₆H₂₄N₄O₄ [M + H]⁺ 457.1, found 457.0. |
| 157 | 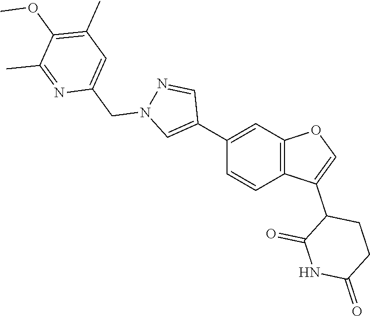 [SMILES: COc1c(C)cc(Cn2cc(-c3ccc4c(C5CCC(=O)NC5=O)coc4c3)cn2)nc1C] | ¹HNMR (400 MHz, DMSO-d₆) δ 10.91 (br s, 1H), 8.33 (s, 1H), 8.00 (s, 1H), 7.85 (s, 1H), 7.81 (s, 1H), 7.59-7.46 (m, 2H), 6.85 (s, 1H), 5.30 (s, 2H), 4.13 (dd, J = 4.8, 12.0 Hz, 1H), 3.66 (s, 3H), 2.80-2.69 (m, 1H), 2.64-2.54 (m, 1H), 2.39 (s, 3H), 2.32 (br dd, J = 4.0, 12.0 Hz, 1H), 2.19 (s, 3H), 2.13 (dt, J = 4.4, 8.8 Hz, 1H). MS: (ESI) m/z calculated for C₂₅H₂₄N₄O₄ [M + H]⁺ 445.1, found 445.3 . |
| 158 | 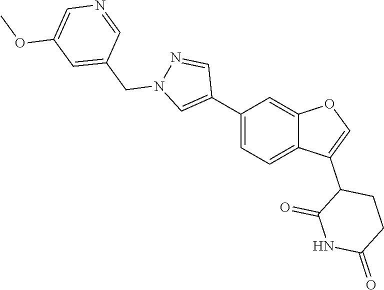 [SMILES: COc1cncc(Cn2cc(-c3ccc4c(C5CCC(=O)NC5=O)coc4c3)cn2)c1] | ¹H NMR (400 MHz, DMSO-d₆) δ 10.88 (s, 1 H), 8.37 (s, 1 H), 8.24 (d, J = 2.8 Hz, 1 H), 8.14 (d, J = 1.6 Hz, 1 H), 8.00 (s, 1 H), 7.85 (s, 1 H), 7.79 (d, J = 0.8 Hz, 1 H), 7.57-7.52 (m, 1 H), 7.50- 7.46 (m, 1 H), 7.34-7.30 (m, 1 H), 5.38 (s, 2 H), 4.12 (dd, J = 12.0, 4.8 Hz, 1 H), 3.81 (s, 3 H), 2.79- 2.69 (m, 1 H), 2.57 (m, 1 H), 2.32 (m, 1 H), 2.17-2.07 (m, 1 H). MS: (ESI) m/z calculated for C₂₃H₂₀N₄O₄ [M + H]⁺ 417.1, found 417.1. |
| 159 | 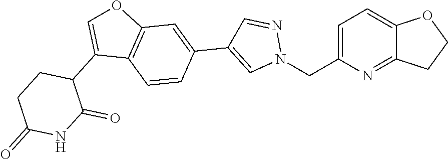 [SMILES: O=C1CCC(c2coc3cc(-c4cnn(Cc5ccc6c(n5)CCO6)c4)ccc23)C(=O)N1] | ¹H NMR (400 MHz, DMSO-d₆) δ 10.90 (br s, 1H), 8.31 (s, 1H), 7.97 (s, 1H), 7.88-7.72 (m, 2H), 7.60-7.41 (m, 2H), 7.11 (d, J = 8.4 Hz, 1H), 6.98 (d, J = 8.4 Hz, 1H), 5.32 (s, 2H), 4.62 (t, J = 8.8 Hz, 2H), 4.12 (dd, J = 4.8, 12.0 Hz, 1H), 3.25-3.22 (m, 2H), 2.86-2.71 (m, 1H), 2.62-2.56 (m, 1H), 2.32 (br dd, J = 4.8, 12.4 Hz, 1H), 2.13 (dt, J = 4.0, 8.8 Hz, 1H). MS: (ESI) m/z calculated for C₂₄H₂₀N₄O₄ [M + H]+ 429.1, found 429.1. |
| 160 | 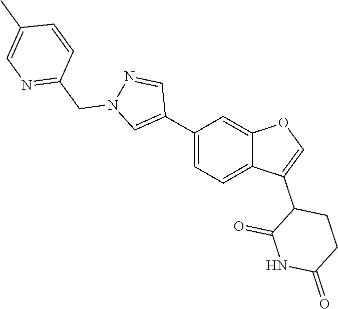 [SMILES: Cc1ccc(Cn2cc(-c3ccc4c(C5CCC(=O)NC5=O)coc4c3)cn2)nc1] | ¹H NMR (400 MHz, DMSO-d₆) δ 10.90 (s, 1H), 8.40-8.32 (m, 2H), 8.03-7.98 (m, 1H), 7.89- 7.78 (m, 2H), 7.64-7.46 (m, 3H), 7.06 (d, J = 8.0 Hz, 1H), 5.42-5.37 (m, 2H), 4.13 (dd, J = 4.8, 12.0 Hz, 1H), 2.81-2.69 (m, 1H), 2.63- 2.54 (m, 1H), 2.41-2.30 (m, 1H), 2.28 (s, 3H), 2.17-2.08 (m, 1H). MS: (ESI) m/z calculated for C₂₃H₂₀N₄O₃ [M + H]⁺ 401.1, found 401.2. |
| 161 | 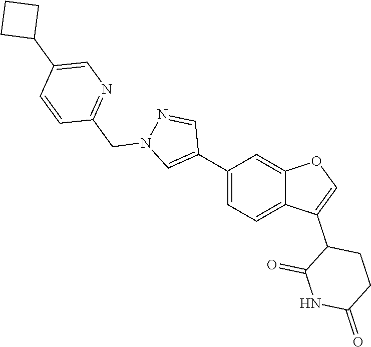 [SMILES: O=C1CCC(c2coc3cc(-c4cnn(Cc5ccc(C6CCC6)cn5)c4)ccc23)C(=O)N1] | ¹H NMR (400 MHz, DMSO-d₆) δ 10.90 (s, 1H), 8.40 (s, 1H), 8.35 (s, 1H), 8.00 (s, 1H), 7.86 (s, 1H), 7.81 (s, 1H), 7.69 (dd, J = 7.6, 2.4 Hz, 1H), 7.53-7.58 (m, 1H), 7.47-7.52 (m, 1H), 7.11 (d, J = 8.0 Hz, 1H), 5.40 (s, 2H), 4.13 (dd, J = 12.4, 5.2 Hz, 1H), 2.64-2.69 (m, 2H), 2.59-2.61 (m, 1H), 2.26-2.37 (m, 3H), 2.06-2.17 (m, 3H), 1.90- 2.04 (m, 1H), 1.76-1.89 (m, 1H). LCMS: calc. for C₂₆H₂₄N₄O₃: 440.2, found: [M + H]⁺ 441.1 |
| 162 | 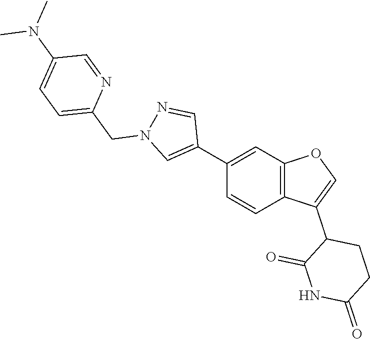 [SMILES: CN(C)c1ccc(Cn2cc(-c3ccc4c(C5CCC(=O)NC5=O)coc4c3)cn2)nc1] | ¹HNMR (400 MHz, METHANOL-d₄) δ 8.24 (s, 1H), 8.06 (d, J = 2.8 Hz, 1H), 7.98 (s, 1H), 7.78 (br d, J = 2.8 Hz, 1H), 7.73-7.70 (m, 2H), 7.66 (d, J = 9.6 Hz, 1H), 7.58-7.54 (m, 1H), 7.49 (d, J = 1.2 Hz, 1H), 5.61 (s, 2H), 4.17-4.09 (m, 1H), 3.12 (s, 6H), 2.86-2.67 (m, 2H), 2.46-2.25 (m, 2H). MS: (ESI) m/z calculated for C₂₄H₂₃N₅O₃ [M + H]⁺ 430.1, found 430.2. |
| 163 | 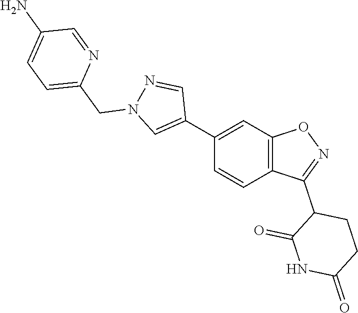 [SMILES: Nc1ccc(Cn2cc(-c3ccc4c(C5CCC(=O)NC5=O)noc4c3)cn2)nc1] | ¹H NMR (400 MHz, DMSO-d₆) δ 10.91 (s, 1 H), 8.26 (s, 1 H), 7.96 (s, 1 H), 7.89 (d, J = 2.4 Hz, 1 H), 7.86 (s, 1 H), 7.80 (s, 1 H), 7.56-7.52 (m, 1 H), 7.51-7.46 (m, 1 H), 6.97-6.92 (m, 1 H), 6.91-6.87 (m, 1 H), 5.34 (s, 2 H), 5.21 (s, 2 H), 4.13 (dd, J = 12.0, 4.8 Hz, 1 H), 2.80-2.70 (m, 1 H), 2.58 (m, 1 H), 2.33 (m, 1 H), 2.18-2.09 (m, 1 H). MS: (ESI) m/z calculated for C₂₂H₁₉N₅O₃ [M + H]⁺ 402.1, found 402.0. |
| 164 | 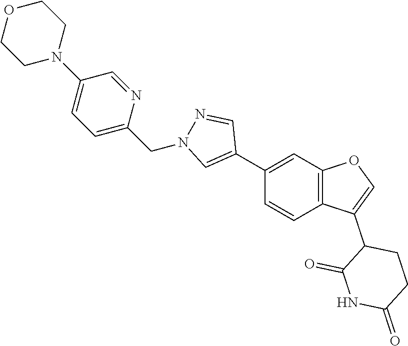 [SMILES: O=C1CCC(c2coc3cc(-c4cnn(Cc5ccc(N6CCOCC6)cn5)c4)ccc23)C(=O)N1] | ¹H NMR (400 MHz, DMSO-d₆) δ 10.90 (s, 1H), 8.31 (s, 1H), 8.25 (d, J = 2.8 Hz, 1H), 7.97 (s, 1H), 7.85 (s, 1H), 7.80 (d, J = 1.0 Hz, 1H), 7.58- 7.43 (m, 2H), 7.33 (dd, J = 3.6, 8.8 Hz, 1H), 7.10 (d, J = 8.8 Hz, 1H), 5.32 (s, 2H), 4.13 (dd, J = 4.8, 11.9 Hz, 1H), 3.78-3.69 (m, 4H), 3.17- 3.12 (m, 4H), 2.80-2.69 (m, 1H), 2.63-2.54 (m, 1H), 2.37-2.32 (m, 1H), 2.21-2.08 (m, 1H). MS: (ESI) m/z calculated for C₂₆H₂₅N₅O₄ [M + H]⁺ 472.2. |
| 165 | 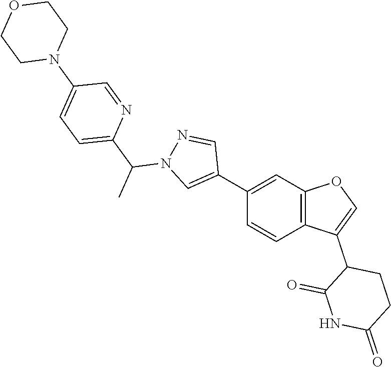 [SMILES: CC(c1ccc(N2CCOCC2)cn1)n1cc(-c2ccc3c(C4CCC(=O)NC4=O)coc3c2)cn1] | ¹HNMR (400 MHz, DMSO-d₆) δ 10.90 (s, 1H), 8.45 (s, 1H), 8.29 (d, J = 2.4 Hz, 1H), 8.01 (s, 1H), 7.86 (s, 1H), 7.82 (s, 1H), 7.67 (br d, J = 8.4 Hz, 1H), 7.57-7.49 (m, 2H), 7.37 (br d, J = 8.8 Hz, 1H), 5.81-5.74 (m, 1H), 4.15-4.11 (m, 1H), 3.75-3.72 (m, 4H), 3.26-3.22 (m, 4H), 2.79-2.69 (m, 1H), 2.61-2.55 (m, 1H), 2.38- 2.31 (m, 1H), 2.16-2.08 (m, 1H), 1.90 (d, J = 7.2 Hz, 3H). MS: (ESI) m/z calculated for C₂₇H₂₇ N₅O₄ [M + H]⁺ 486.2, found 486.2. |
| 166 | 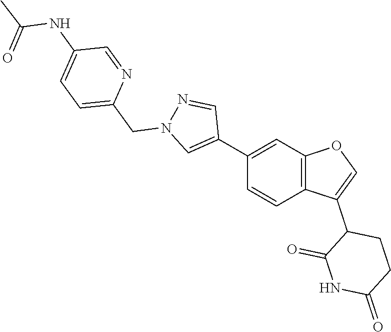 [SMILES: CC(=O)Nc1ccc(Cn2cc(-c3ccc4c(C5CCC(=O)NC5=O)coc4c3)cn2)nc1] | ¹H NMR (400 MHz, DMSO-d₆) δ 10.89 (s, 1H), 10.17 (s, 1H), 8.68 (d, J = 2.4 Hz, 1H), 8.33 (s, 1H), 8.01-8.00 (m, 1H), 7.98 (d, J = 2.4 Hz, 1H), 7.86 (s, 1H), 7.81 (d, J = 0.8 Hz, 1H), 7.57- 7.53 (m, 1H), 7.51-7.48 (m, 1H), 7.15 (d, J = 8.4 Hz, 1H), 5.38 (s, 2H), 4.15-4.10 (m, 1H), 2.76-2.70 (m, 1H), 2.62-2.58 (m, 1H), 2.35- 2.30 (m, 1H), 2.16-2.10 (m, 1H), 2.06 (s, 3H). MS: (ESI) m/z calculated for C₂₄H₂₁N₅O₄ [M + H]⁺ 444.1, found 444.2. |
| 167 | 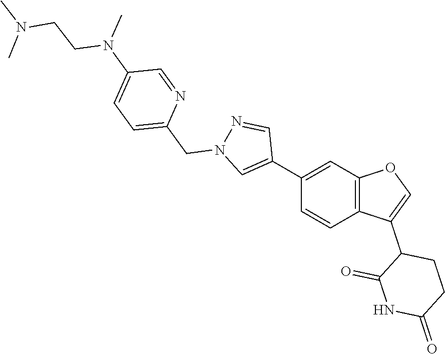 [SMILES: CN(C)CCN(C)c1ccc(Cn2cc(-c3ccc4c(C5CCC(=O)NC5=O)coc4c3)cn2)nc1] | ¹H NMR (400 MHz, DMSO-d₆) δ 10.90 (s, 1 H), 8.30-8.27 (m, 1 H), 8.25 (s, 1 H), 8.03-7.99 (m, 1 H), 7.96 (s, 1 H), 7.87-7.84 (m, 1 H), 7.79 (s, 1 H), 7.57-7.51 (m, 1 H), 7.51-7.46 (m, 1 H), 7.09-7.02 (m, 2 H), 5.27 (s, 2 H), 4.12 (dd, J = 12.0, 4.8 Hz, 1 H), 3.43 (t, J = 7.2 Hz, 2 H), 2.91 (s, 3 H), 2.79-2.70 (m, 1 H), 2.58 (m, 1 H), 2.39-2.34 (m, 2 H), 2.34-2.27 (m, 1 H), 2.19- 2.15 (m, 6 H), 2.14-2.09 (m, 1 H). MS: (ESI) m/z calculated for C₂₇H₃₀N₆O₃ [M + H]⁺ 487.2, found 487.3. |
| 168 | 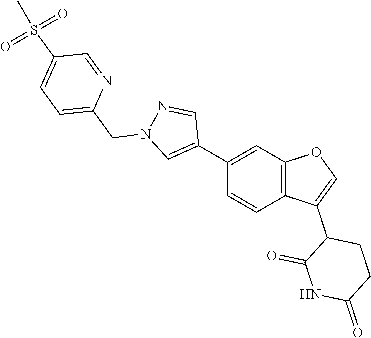 [SMILES: CS(=O)(=O)c1ccc(Cn2cc(-c3ccc4c(C5CCC(=O)NC5=O)coc4c3)cn2)nc1] | ¹H NMR (400 MHz, DMSO-d₆) δ 10.90 (s, 1H), 9.04 (d, J = 2.0 Hz, 1H), 8.43 (s, 1H), 8.32 (dd, J = 2.4, 8.4 Hz, 1H), 8.06 (s, 1H), 7.85 (d, J = 16.0 Hz, 2H), 7.59-7.47 (m, 2H), 7.37 (d, J = 8.4 Hz, 1H), 5.61 (s, 2H), 4.13 (dd, J = 4.8, 12.0 Hz, 1H), 3.31 (s, 3H), 2.81-2.70 (m, 1H), 2.63-2.55 (m, 1H), 2.33 (dq, J = 4.4, 12.4 Hz, 1H), 2.18-2.09 (m, 1H). MS: (ESI) m/z calculated for C₂₃H₂₀N₄O₅S [M + H]⁺ 465.1, found 465.1. |
| 169 | 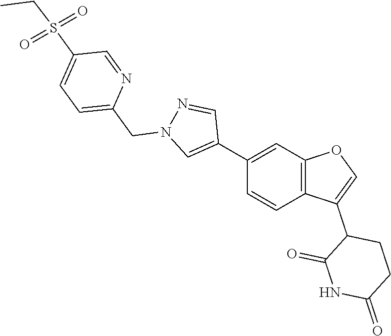 [SMILES: CCS(=O)(=O)c1ccc(Cn2cc(-c3ccc4c(C5CCC(=O)NC5=O)coc4c3)cn2)nc1] | ¹H NMR (400 MHz, DMSO-d₆) δ 10.90 (s, 1H), 9.00 (d, J = 2.0 Hz, 1H), 8.43 (s, 1H), 8.29 (dd, J = 2.4, 8.4 Hz, 1H), 8.07 (s, 1H), 7.85 (d, J = 16.0 Hz, 2H), 7.62-7.47 (m, 2H), 7.35 (d, J = 8.4 Hz, 1H), 5.62 (s, 2H), 4.13 (dd, J = 4.8, 12.0 Hz, 1H), 3.42-3.40 (m, 2H), 2.80-2.69 (m, 1H), 2.63- 2.56 (m, 1H), 2.39-2.30 (m, 1H), 2.19-2.09 (m, 1H), 1.12 (t, J = 7.2 Hz, 3H). MS: (ESI) m/z calculated for C₂₄H₂₂N₄O₅S [M + H]⁺ 479.1, found 479.2. |
| 170 | 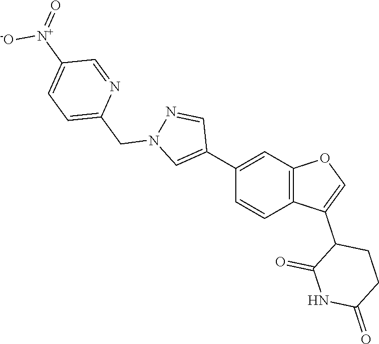 | ¹H NMR (400 MHz, DMSO-d₆) δ 10.91 (s, 1 H), 9.35 (s, 1 H), 8.61 (d, J = 8.8 Hz, 1 H), 8.59 (s, 1 H), 8.43 (s, 1 H), 8.08 (s, 1 H), 7.85 (d, J = 8.0 Hz, 1 H), 7.57 (d, J = 4.0 Hz, 1 H), 7.52 (d, J = 1.2 Hz, 1 H), 7.50-7.35 (m, 1 H), 5.64 (s, 2 H), 4.15-4.11 (m, 1 H), 2.75-2.74 (m, 1 H), 2.61- 2.60 (m, 1 H), 2.34-2.32 (m, 1 H), 2.31-2.14 (m, 1 H). MS: (ESI) m/z calculated for C₂₂H₁₇N₅O₅ [M + H]⁺ 432.1, found 432.1. |
| 171 | 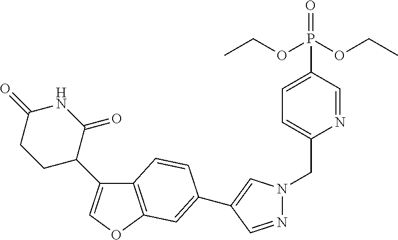 [SMILES: CCOP(=O)(OCC)c1ccc(Cn2cc(-c3ccc4c(C5CCC(=O)NC5=O)coc4c3)cn2)nc1] | ¹H NMR (400 MHz, DMSO-d₆) δ 10.90 (s, 1H), 8.81-2-8.80 (m, 1H), 8.42 (s, 1H), 8.14-8.08 (m, 1H), 8.07-8.05 (m, 1H), 7.89-7.86 (m, 1H), 7.83 (d, J = 0.8 Hz, 1H), 7.60-7.55 (m, 1H), 7.54-7.50 (m, 1H), 7.25-7.23 (m, 1H), 5.56 (s, 2H), 4.17-4.11 (m, 1H), 4.10-4.00 (m, 4H), 2.81-2.71 (m, 1H), 2.62-2.56 (m, 1H), 2.40- 2.28 (m, 1H), 2.19-2.10 (m, 1H), 1.24 (t, J = 7.6 Hz, 6H). MS: (ESI) m/z calculated for C₂₆H₂₇N₄O₆P [M + H]⁺ 523.1, found 523.2. |
| 172 | 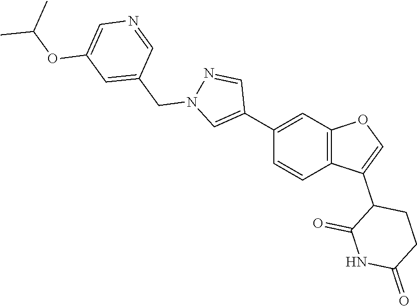 [SMILES: CC(C)Oc1cncc(Cn2cc(-c3ccc4c(C5CCC(=O)NC5=O)coc4c3)cn2)c1] | ¹H NMR (400 MHz, DMSO-d₆) δ 10.90 (br s, 1H), 8.39 (s, 1H), 8.20 (d, J = 2.8 Hz, 1H), 8.12 (d, J = 1.6 Hz, 1H), 8.01 (s, 1H), 7.87 (s, 1H), 7.81 (s, 1H), 7.59-7.53 (m, 1H), 7.51-7.47 (m, 1H), 7.30-7.24 (m, 1H), 5.38 (s, 2H), 4.67 (td, J = 6.0, 12.0 Hz, 1H), 4.14 (dd, J = 4.8, 12.0 Hz, 1H), 2.73 (br dd, J = 5.6, 12.0 Hz, 1H), 2.63- 2.59 (m, 1H), 2.36-2.30 (m, 1H), 2.17-2.10 (m, 1H), 1.27 (d, J = 6.0 Hz, 6H). MS: (ESI) m/z calculated for C₂₅H₂₄N₄O₄ [M + H]⁺ 445.1, found 445.5. |
| 173 | 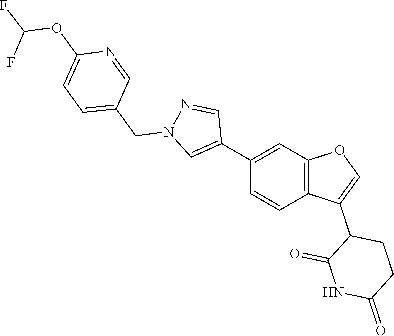 [SMILES: O=C1CCC(c2coc3cc(-c4cnn(Cc5ccc(OC(F)F)nc5)c4)ccc23)C(=O)N1] | ¹H NMR (400 MHz, DMSO-d₆) δ ¹H NMR (400 MHz, DMSO-d₆) δ 10.90 (s, 1H), 8.37 (s, 1H), 8.28 (d, J = 2.0 Hz, 1H), 8.00 (s, 1H), 7.90-7.85 (m, 2H), 7.79 (d, J = 0.8 Hz, 1H), 7.69 (s, 1H), 7.61-7.43 (m, 2H), 7.09 (d, J = 8.4 Hz, 1H), 5.38 (s, 2H), 4.13 (dd, J = 4.0, 12.0 Hz, 1H), 2.81- 2.69 (m, 1H), 2.62-2.56 (m, 1H), 2.32 (br dd, J = 4.4, 12.0 Hz, 1H), 2.12 (td, J = 4.0, 8.8 Hz, 1H). |
| 174 | 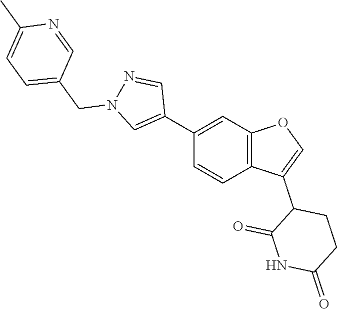 [SMILES: Cc1ccc(Cn2cc(-c3ccc4c(C5CCC(=O)NC5=O)coc4c3)cn2)cn1] | ¹H NMR (400 MHz, DMSO-d6) δ 10.89 (s, 1 H), 8.44 (s, 1 H), 8.35 (s, 1 H), 7.98 (s, 1 H), 7.86 (s, 1 H), 7.79 (d, J = 0.8 Hz, 1 H), 7.60 (dd, J = 8.0, 2.0 Hz, 1 H), 7.57-7.53 (m, 1 H), 7.50-7.45 (m, 1 H), 7.24 (d, J = 8.0 Hz, 1 H), 5.34 (s, 2 H), 4.16- 4.09 (m, 1 H), 2.78-2.69 (m, 1 H), 2.62-2.58 (m, 1 H), 2.44 (s, 3 H), 2.35-2.30 (m, 1 H), 2.17- 2.08 (m, 1 H). MS: (ESI) m/z calculated for C₂₃H₂₀N₄O₃ [M + H]⁺ 401.1, found 401.1. |
| 175 | 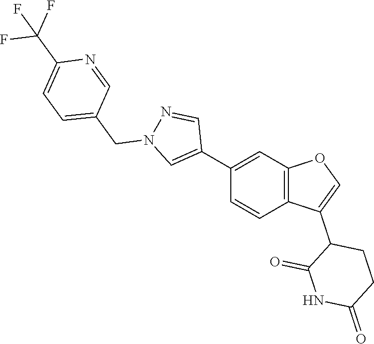 [SMILES: O=C1CCC(c2coc3cc(-c4cnn(Cc5ccc(C(F)(F)F)nc5)c4)ccc23)C(=O)N1] | ¹H NMR (400 MHz, DMSO-d₆) δ 10.90 (s, 1H), 8.74 (s, 1H), 8.41 (s, 1H), 8.04 (s, 1H), 7.95- 7.89 (m, 2H), 7.86 (s, 1H), 7.80 (d, J = 0.8 Hz, 1H), 7.58-7.53 (m, 1H), 7.51-7.46 (m, 1H), 5.54 (s, 2H), 4.12 (dd, J = 4.8, 12.0 Hz, 1H), 2.82- 2.66 (m, 1H), 2.57 (td, J = 4.0, 16.0 Hz, 1H), 2.32 (br dd, J = 4.0, 12.8 Hz, 1H), 2.17-2.08 (m, 1H). ¹⁹F NMR (376 MHz, DMSO-d₆) δ −66.360. MS: (ESI) m/z calculated for C₂₃H₁₇F₃N₄O₃ [M + H]⁺ 455.1, found 455.3. |
| 176 | 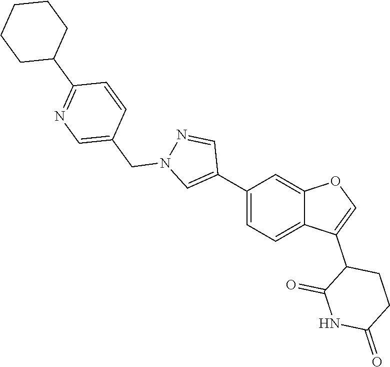 [SMILES: O=C1CCC(c2coc3cc(-c4cnn(Cc5ccc(C6CCCCC6)nc5)c4)ccc23)C(=O)N1] | ¹H NMR (400 MHz, DMSO-d₆) δ 10.90 (s, 1H), 8.60 (s, 1H), 8.39 (s, 1H), 8.02 (s, 1H), 7.90-7.97 (m, 1H), 7.86 (s, 1H), 7.80 (s, 1H), 7.49-7.59 (m, 2H), 7.47 (d, J = 8.0 Hz, 1H), 5.43 (s, 2H), 4.09- 4.21 (m, 1H), 2.70-2.81 (m, 2H), 2.51-2.64 (m, 1H), 2.32-2.45 (m, 1H), 2.04-2.17 (m, 1H), 1.72- 1.91 (m, 4H), 1.63-1.71 (m, 1H), 1.42-1.57 (m, 2H), 1.26-1.40 (m, 2H), 1.12-1.24 (m, 1H). LCMS: calc. for C₂₈H₂₈N₄O₃: 468.2, found: [M + H]⁺ 469.3. |
| 177 | 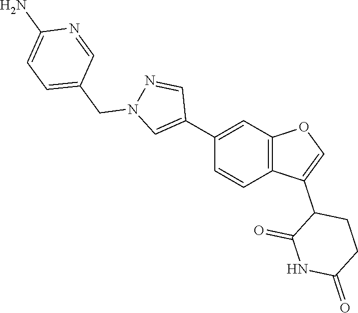 [SMILES: Nc1ccc(Cn2cc(-c3ccc4c(C5CCC(=O)NC5=O)coc4c3)cn2)cn1] | ¹H NMR (400 MHz, DMSO-d₆) δ 10.90 (s, 1 H), 8.26 (s, 1 H), 8.13 (s, 1 H), 7.94 (s, 2 H), 7.85 (s, 1 H), 7.78 (s, 1 H), 7.57-7.51 (m, 1 H), 7.49- 7.44 (m, 1 H), 7.39 (dd, J-8.8, 2.4 Hz, 1 H), 6.43 (d, J = 8.8 Hz, 1 H), 6.03 (s, 2 H), 5.16-5.10 (m, 2 H), 4.12 (dd, J = 11.6, 4.8 Hz, 1 H), 2.80-2.69 (m, 1 H), 2.62-2.54 (m, 1 H), 2.32 (m, 1 H), 2.17-2.08 (m, 1 H). MS: (ESI) m/z calculated for C₂₂H₁₉N₅O₃ [M + H]⁺ 402.1, found 402.1. |
| 178 | 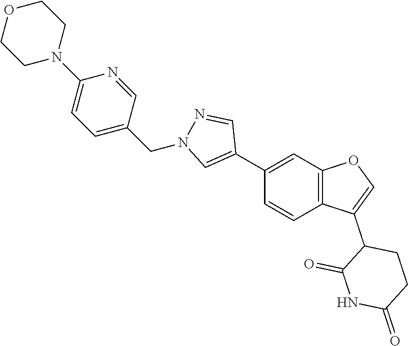 [SMILES: O=C1CCC(c2coc3cc(-c4cnn(Cc5ccc(N6CCOCC6)nc5)c4)ccc23)C(=O)N1] | ¹H NMR (400 MHz, DMSO-d₆) δ 10.90 (s, 1H), 8.30 (s, 1H), 8.17 (d, J = 2.4 Hz, 1H), 7.95 (s, 1H), 7.85 (s, 1H), 7.78 (d, J = 0.8 Hz, 1H), 7.59- 7.52 (m, 2H), 7.46 (dd, J = 1.2, 8.0 Hz, 1H), 6.82 (d, J = 8.8 Hz, 1H), 5.21 (s, 2H), 4.12 (dd, J = 4.8, 12.0 Hz, 1H), 3.70-3.64 (m, 4H), 3.43- 3.40 (m, 4H), 2.81-2.68 (m, 1H), 2.64-2.56 (m, 1H), 2.32 (br dd, J = 4.0, 12.4 Hz, 1H), 2.17- 2.05 (m, 1H). MS: (ESI) m/z calculated for C₂₆H₂₅N₅O₄ [M + H]⁺ 472.1, found 471.9. |
| 179 | 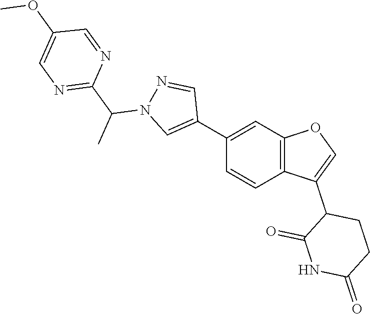 [SMILES: COc1cnc(C(C)n2cc(-c3ccc4c(C5CCC(=O)NC5=O)coc4c3)cn2)nc1] | ¹H NMR (400 MHz, DMSO-d₆) δ 11.03-10.74 (m, 1H), 8.54 (s, 2H), 8.38 (s, 1H), 7.91 (s, 1H), 7.83 (d, J = 12.8 Hz, 2H), 7.57-7.49 (m, 2H), 5.70 (q, J = 6.8 Hz, 1H), 4.13 (dd, J = 4.8, 11.8 Hz, 1H), 3.92-3.87 (m, 3H), 2.79-2.70 (m, 1H), 2.62-2.55 (m, 1H), 2.33 (br dd, J = 4.0, 12.0 Hz, 1H), 2.13 (td, J = 4.0, 8.8 Hz, 1H), 1.88 (d, J = 7.2 Hz, 3H) MS: (ESI) m/z calculated for C₂₃H₂₁N₅O₄ [M + H]⁺ 432.4, found 432.2. |
| 180 | 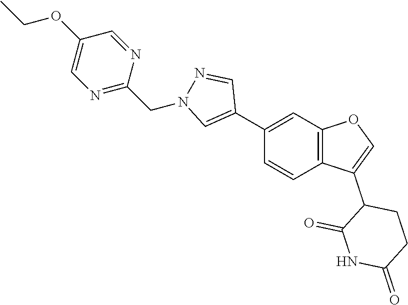 [SMILES: CCOc1cnc(Cn2cc(-c3ccc4c(C5CCC(=O)NC5=O)coc4c3)cn2)nc1] | ¹H NMR (400 MHz, DMSO-d₆) δ 10.90 (s, 1H), 8.52 (s, 2H), 8.32 (s, 1H), 7.95 (s, 1H), 7.86 (s, 1H), 7.81 (s, 1H), 7.57-7.53 (m, 1H), 7.52-7.47 (m, 1H), 5.48 (s, 2H), 4.21-4.15 (m, 2H), 4.14- 4.10 (m, 1H), 2.80-2.69 (m, 1H), 2.58 (d, J = 4.0, 16.0 Hz, 1H), 2.39-2.29 (m, 1H), 2.17- 2.08 (m, 1H), 1.34 (t, J = 8.0 Hz, 3H) MS: (ESI) m/z calculated for C₂₃H₂₁N₅O₄ [M + H]⁺ 432.1, found 432.2. |
| 181 | 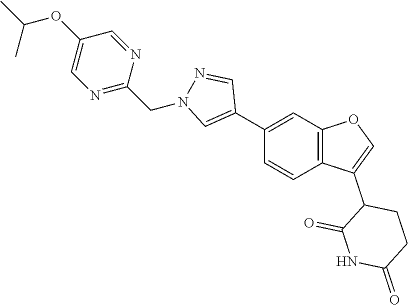 [SMILES: CC(C)Oc1cnc(Cn2cc(-c3ccc4c(C5CCC(=O)NC5=O)coc4c3)cn2)nc1] | ¹H NMR (400 MHz, DMSO-d₆) δ 10.90 (s, 1H), 8.51 (s, 2H), 8.33 (s, 1H), 7.96 (s, 1H), 7.86 (s, 1H), 7.81 (s, 1H), 7.58-7.46 (m, 2H), 5.48 (s, 2H), 4.78 (td, J = 6.0, 12.0 Hz, 1H), 4.13 (dd, J = 4.8, 12.0 Hz, 1H), 2.75 (ddd, J = 4.8, 12.0, 17.3 Hz, 1H), 2.60 (br t, J = 4.0 Hz, 1H), 2.40-2.27 (m, 1H), 2.14 (dt, J = 4.4, 8.8 Hz, 1H), 1.29 (d, J = 6.0 Hz, 6H). (ESI) m/z calculated for C₂₄H₂₃N₅O₄ [M + H]⁺ 446.1, found 446.0. |
| 182 | 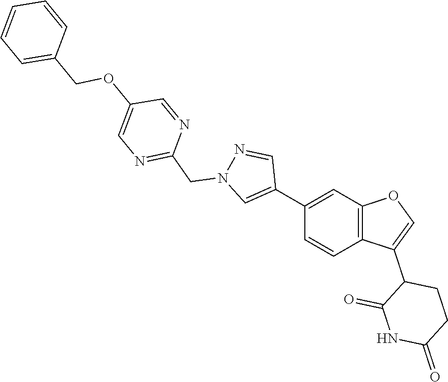 [SMILES: O=C1CCC(c2coc3cc(-c4cnn(Cc5ncc(OCc6ccccc6)cn5)c4)ccc23)C(=O)N1] | ¹H NMR (400 MHz, DMSO-d₆) δ 10.95-10.85 (m, 1H), 8.60 (s, 2H), 8.32 (s, 1H), 7.95 (s, 1H), 7.89-7.78 (m, 2H), 7.62-7.24 (m, 7H), 5.49 (s, 2H), 5.31-5.22 (m, 2H), 4.17-4.09 (m, 1H), 2.78-2.69 (m, 1H), 2.60 (br d, J = 4.0 Hz, 1H), 2.36-2.30 (m, 1H), 2.13 (td, J = 4.0, 8.8 Hz, 1H). MS: (ESI) m/z calculated for C₂₈H₂₃N₅O₄ [M + H]⁺ 494.1, found 494.2. |
| 183 | 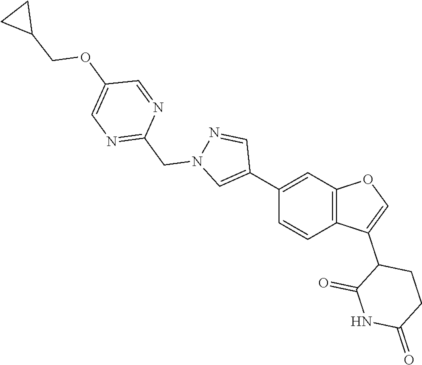 [SMILES: O=C1CCC(c2coc3cc(-c4cnn(Cc5ncc(OCC6CC6)cn5)c4)ccc23)C(=O)N1] | ¹H NMR (400 MHz, DMSO-d₆) δ 10.90 (br s, 1H), 8.52 (s, 2H), 8.32 (s, 1H), 7.95 (s, 1H), 7.89- 7.73 (m, 2H), 7.63-7.46 (m, 2H), 5.48 (s, 2H), 4.13 (dd, J = 4.4, 12.0 Hz, 1H), 3.98 (d, J = 6.8 Hz, 2H), 2.85-2.68 (m, 1H), 2.58 (d, J = 16. Hz, 1H), 2.33 (dd, J = 3.2, 12.0 Hz, 1H), 2.24-2.09 (m, 1H), 1.23 (s, 1H), 0.58 (d, J = 6.8 Hz, 2H), 0.33 (d, J = 4.4 Hz, 2H). (ESI) m/z calculated for C₂₅H₂₃N₅O₄ [M + H]⁺ 458.1, found 458.1. |
| 184 | 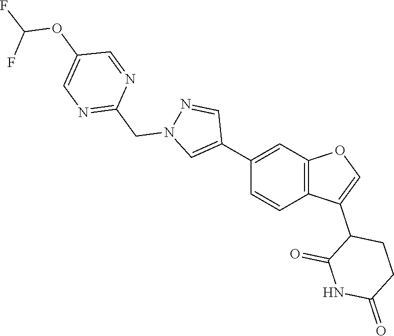 [SMILES: O=C1CCC(c2coc3cc(-c4cnn(Cc5ncc(OC(F)F)cn5)c4)ccc23)C(=O)N1] | ¹H NMR (400 MHz, CD₃OD) δ 8.68 (s, 2H), 8.21 (s, 1H), 7.92 (s, 1H), 7.71 (s, 2H), 7.60- 7.45 (m, 2H), 7.01 (t, J = 2.4 Hz, 1H), 5.63 (s, 2H), 4.14 (dd, J = 4.8, 12.0 Hz, 1H), 2.86-2.68 (m, 2H), 2.47-2.24 (m, 2H). ¹⁹F NMR (400 MHz, CD₃OD) δ −84.50. (ESI) m/z calculated for C₂₂H₁₇F₂N₅O₄ [M + H]⁺ 454.1, found 454.2. |
| 185 | 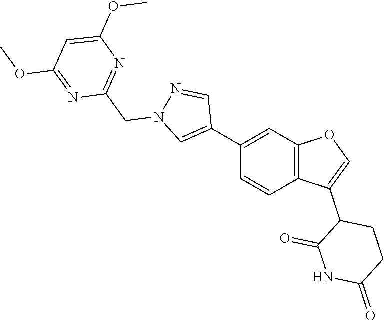 [SMILES: COc1cc(OC)nc(Cn2cc(-c3ccc4c(C5CCC(=O)NC5=O)coc4c3)cn2)n1] | ¹H NMR (400 MHz, DMSO-d₆) δ 10.90 (s, 1H), 8.35 (s, 1H), 7.99 (s, 1H), 7.86 (s, 1H), 7.82 (s, 1H), 7.74-7.36 (m, 2H), 6.18 (s, 1H), 5.38 (s, 2H), 4.13 (dd, J = 4.8, 11.6 Hz, 1H), 3.81 (s, 6H), 2.74 (dt, J = 6.0, 11.6 Hz, 1H), 2.67-2.56 (m, 1H), 2.39-2.28 (m, 1H), 2.23-2.01 (m, 1H). MS: (ESI) m/z calculated for C₂₃H₂₁N₅O₅ [M + H]⁺ 448.1, found 448.1. |
| 186 | 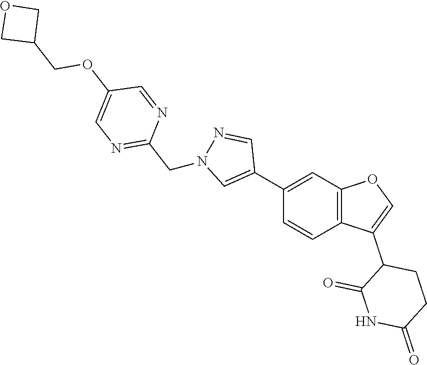 [SMILES: O=C1CCC(c2coc3cc(-c4cnn(Cc5ncc(OCC6COC6)cn5)c4)ccc23)C(=O)N1] | ¹H NMR (400 MHz, CD₃OD-d₄) δ 8.51 (s, 2H), 8.18 (s, 1H), 7.90 (s, 1H), 7.70 (s, 2H), 7.60- 7.41 (m, 2H), 5.55 (s, 2H), 4.85 (br s, 2H), 4.62- 4.58 (m, 2H), 4.37 (d, J = 6.4 Hz, 2H), 4.13 (dd, J = 4.8, 11.6 Hz, 1H), 3.53-3.43 (m, 1H), 2.91- 2.62 (m, 2H), 2.49-2.25 (m, 2H). MS: (ESI) m/z calculated for C₂₅H₂₃N₅O₅ [M + H]⁺ 474.1, found 474.1. |
| 187 | 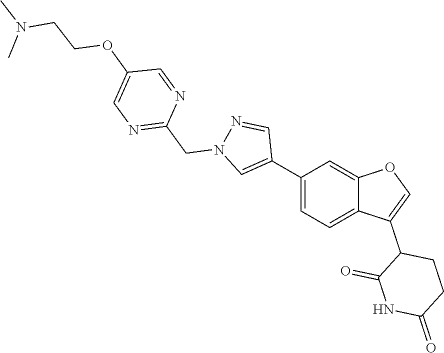 [SMILES: CN(C)CCOc1cnc(Cn2cc(-c3ccc4c(C5CCC(=O)NC5=O)coc4c3)cn2)nc1] | ¹H NMR (400 MHz, DMSO-d₆) δ 10.90 (s, 1H), 8.54 (s, 2H), 8.32 (s, 1H), 7.97-7.95 (m, 1H), 7.86 (s, 1H), 7.81 (d, J = 0.8 Hz, 1H), 7.57-7.53 (m, 1H), 7.51-7.47 (m, 1H), 5.48 (s, 2H), 4.20 (t, J = 5.6 Hz, 2H), 4.13 (m, 1H), 2.75 (m, 1H), 2.63 (t, J = 5.6 Hz, 2H), 2.61-2.54 (m, 1H), 2.38- 2.27 (m, 1H), 2.20 (s, 6H), 2.17-2.09 (m, 1H). MS: (ESI) m/z calculated for C₂₅H₂₆N₆O₄ [M + H]⁺ 475.2, found 475.2. |
| 188 | 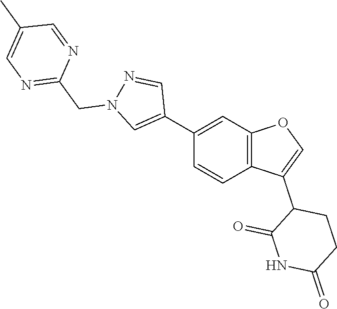 [SMILES: Cc1cnc(Cn2cc(-c3ccc4c(C5CCC(=O)NC5=O)coc4c3)cn2)nc1] | ¹H NMR (400 MHz, DMSO-d₆) δ 10.90 (s, 1H), 8.64 (s, 2H), 8.33 (s, 1H), 7.96 (s, 1H), 7.86 (s, 1H), 7.81 (s, 1H), 7.54 (s, 1H), 7.51 (d, J = 2.0 Hz, 1H), 5.51 (s, 2H), 4.13 (dd, J = 4.8, 12.0 Hz, 1H), 2.81-2.66 (m, 1H), 2.63-2.53 (m, 1H), 2.33 (dd, J = 4.0, 12.0 Hz, 1H), 2.26 (s, 3H), 2.19- 2.05 (m, 1H). MS: (ESI) m/z calculated for C₂₂H₁₉N₅O₃ [M + H]⁺ 402.1, found 402.5. |
| 189 | 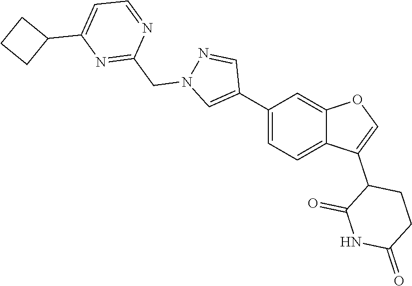 [SMILES: O=C1CCC(c2coc3cc(-c4cnn(Cc5nccc(C6CCC6)n5)c4)ccc23)C(=O)N1] | ¹H NMR (400 MHz, DMSO-d₆) δ 10.91 (s, 1H), 8.64 (d, J = 5.2 Hz, 1H), 8.36 (s, 1H), 7.97 (s, 1H), 7.84 (d, J = 18.8 Hz, 2H), 7.49-7.58 (m, 2H), 7.31 (d, J = 5.2 Hz, 1H), 5.53 (s, 2H), 4.13 (dd, J = 12.0, 4.8 Hz, 1 H) 3.62 (t, J = 8.8 Hz, 1H), 2.68-2.82 (m, 1H), 2.54-2.58 (m, 1H), 2.31- 2.37 (m, 1H), 2.19-2.29 (m, 4H), 2.10-2.16 (m, 1H), 1.95-2.04 (m, 1H), 1.79-1.88 (m, 1H). LCMS: calc. for C₂₅H₂₃N₅O₃: 441.2, found: [M + H]⁺ 442.1 |
| 190 | 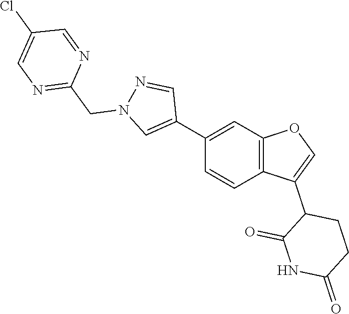 [SMILES: O=C1CCC(c2coc3cc(-c4cnn(Cc5ncc(Cl)cn5)c4)ccc23)C(=O)N1] | ¹H NMR (400 MHz, DMSO-d₆) δ 10.94-10.86 (m, 1H), 8.95 (s, 2H), 8.35 (s, 1H), 7.99 (s, 1H), 7.88-7.78 (m, 2H), 7.59-7.46 (m, 2H), 5.59 (s, 2H), 4.17-4.09 (m, 1H), 2.72-2.66 (m, 1H), 2.61-2.56 (m, 1H), 2.35-2.31 (m, 1H), 2.17- 2.09 (m, 1H) MS: (ESI) m/z calculated for C₂₁H₁₆ClN₅O₃ [M + H]⁺ 422.0, found 422.0. |
| 191 | 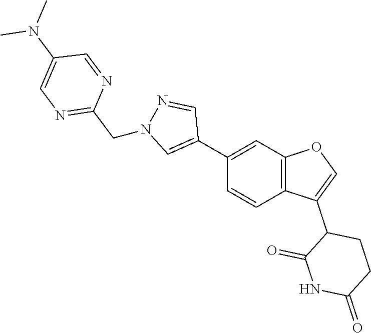 [SMILES: CN(C)c1cnc(Cn2cc(-c3ccc4c(C5CCC(=O)NC5=O)coc4c3)cn2)nc1] | ¹H NMR (400 MHz, DMSO-d₆) δ 10.89 (s, 1H), 8.28 (s, 1H), 8.26 (s, 2H), 7.93-7.92 (m, 1H), 7.85 (s, 1H), 7.79 (d, J = 0.8 Hz, 1H), 7.56-7.53 (m, 1H), 7.50-7.47 (m, 1H), 5.38 (s, 2H), 4.14- 4.10 (m, 1H), 2.94 (s, 6H), 2.80-2.69 (m, 1H), 2.61-2.59 (m, 1H), 2.36-2.30 (m, 1H), 2.17- 2.09 (m, 1H). MS: (ESI) m/z calculated for C₂₃H₂₂N₆O₃ [M + H]⁺ 431.1, found 431.1. |
| 192 | 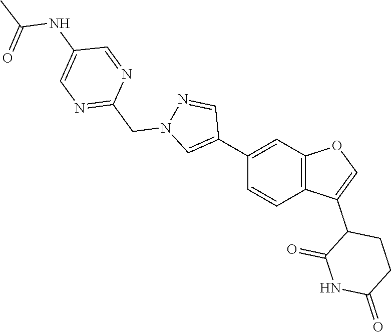 [SMILES: CC(=O)Nc1cnc(Cn2cc(-c3ccc4c(C5CCC(=O)NC5=O)coc4c3)cn2)nc1] | ¹H NMR (400 MHz, DMSO-d₆) δ 10.89 (s, 1H), 10.40 (s, 1H), 8.95 (s, 2H), 8.33 (s, 1H), 7.96 (s, 1H), 7.87-7.85 (m, 1H), 7.81 (s, 1H), 7.57-7.54 (m, 1H), 7.52-7.48 (m, 1H), 5.50 (s, 2H), 4.13 (dd, J = 4.8, 12.0 Hz, 1H), 2.80-2.70 (m, 1H), 2.62-2.56 (m, 1H), 2.38-2.29 (m, 1H), 2.18- 2.11 (m, 1H), 2.09 (s, 3H). MS: (ESI) m/z calculated for C₂₃H₂₀N₆O₄ [M + H]⁺ 445.4, found 445.1. |
| 193 | 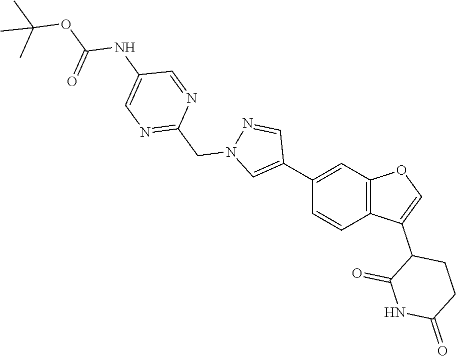 [SMILES: CC(C)(C)OC(=O)Nc1cnc(Cn2cc(-c3ccc4c(C5CCC(=O)NC5=O)coc4c3)cn2)nc1] | ¹H NMR (400 MHz, DMSO-d₆) δ 10.91 (s, 1H), 9.80 (s, 1H), 8.83 (s, 2H), 8.32 (s, 1H), 7.97 (s, 1H), 7.86 (s, 1H), 7.81 (s, 1H), 7.58-7.53 (m, 1H), 7.53-7.48 (m, 1H), 5.48 (s, 2H), 4.17-4.10 (m, 1H), 2.80-2.70 (m, 1H), 2.60 (d, J = 4.0 Hz, 1H), 2.39-2.29 (m, 1H), 2.18-2.10 (m, 1H), 1.48 (s, 9H). MS: (ESI) m/z calculated for C₂₆H₂₆N₆O₅ [M + H]⁺ 503.2, found 503.2. |
| 194 | 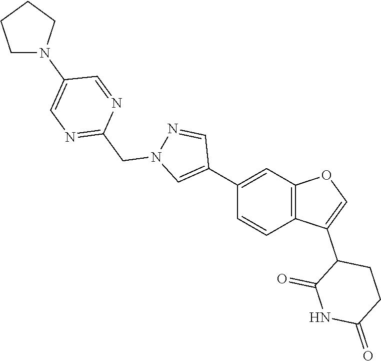 [SMILES: O=C1CCC(c2coc3cc(-c4cnn(Cc5ncc(N6CCCC6)cn5)c4)ccc23)C(=O)N1] | ¹HNMR (400 MHz, DMSO-d₆) δ 10.99-10.82 (m, 1H), 8.27 (s, 1H), 8.09 (s, 2H), 7.93 (s, 1H), 7.86 (s, 1H), 7.80 (s, 1H), 7.59-7.52 (m, 1H), 7.50-7.45 (m, 1H), 5.37 (s, 2H), 4.19-4.03 (m, 1H), 3.26 (br t, J = 6.6 Hz, 4H), 2.80-2.69 (m, 1H), 2.60 (s, 1H), 2.40-2.27 (m, 1H), 2.18-2.10 (m, 1H), 1.97-1.90 (m, 4H). MS: (ESI) m/z calculated for C₂₅H₂₄N₆O₃ [M + H]⁺ 457.1, found 457.2. |
| 195 | 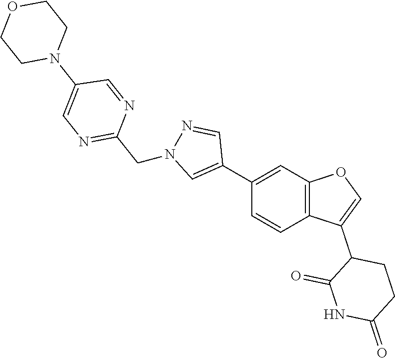 [SMILES: O=C1CCC(c2coc3cc(-c4cnn(Cc5ncc(N6CCOCC6)cn5)c4)ccc23)C(=O)N1] | ¹H NMR (400 MHz, DMSO-d₆) δ 10.91 (s, 1H), 8.48 (s, 2H), 8.31 (s, 1H), 7.94 (s, 1H), 7.90- 7.73 (m, 2H), 7.59-7.46 (m, 2H), 5.42 (s, 2H), 4.13 (dd, J = 4.8, 12.0 Hz, 1H), 3.82-3.67 (m, 4H), 3.26-3.17 (m, 4H), 2.79-2.68 (m, 1H), 2.60 (br s, 1H), 2.33 (dt, J = 8.4, 12.4 Hz, 1H), 2.19-2.06 (m, 1H). MS: (ESI) m/z calculated for C₂₅H₂₄N₆O₄ [M + H]⁺ 473.1, found 473.2. |
| 196 | 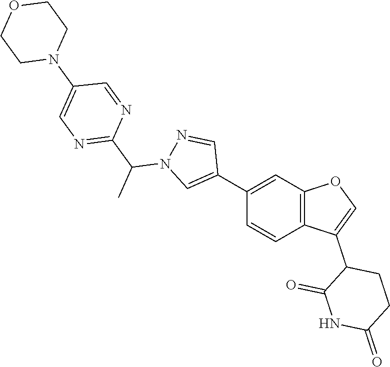 | ¹H NMR (400 MHz, DMSO-d₆) δ 10.94-10.88 (m, 1H), 8.52-8.44 (m, 2H), 8.32-8.27 (m, 1H), 8.01-7.90 (m, 3H), 7.50 (d, J = 12.0 Hz, 1H), 5.70 (q, J = 8.0 Hz, 1H), 4.12 (dd, J = 4.8, 12.4 Hz, 1H), 3.77-3.71 (m, 4H), 3.24-3.20 (m, 4H), 2.75-2.66 (m, 1H), 2.61-2.55 (m, 1H), 2.39-2.31 (m, 1H), 2.15-2.07 (m, 1H), 1.86 (d, J = 7.2 Hz, 3H). ¹⁹F NMR (376 MHz, DMSO-d₆) δ −121.78. MS: (ESI) m/z calculated for C₂₆H₂₅FN₆O₄ [M + H]⁺ 505.2, found 505.2. |
| 197 | 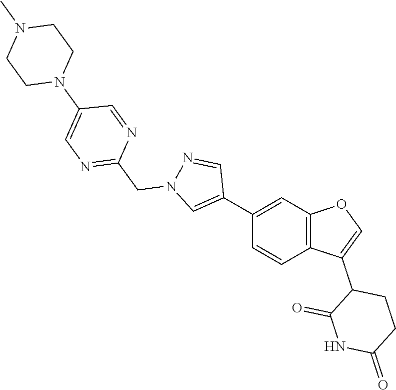 [SMILES: CN1CCN(c2cnc(Cn3cc(-c4ccc5c(C6CCC(=O)NC6=O)coc5c4)cn3)nc2)CC1] | ¹H NMR (400 MHz, DMSO-d₆) δ 10.95-10.88 (m, 1H), 8.59-8.52 (m, 2H), 8.36-8.28 (m, 1H), 7.98 (s, 1H), 7.86 (s, 1H), 7.84-7.79 (m, 1H), 7.58-7.47 (m, 2H), 5.44 (s, 2H), 4.17-4.09 (m, 1H), 4.00 (br d, J = 12.0 Hz, 2H), 3.53 (d, J = 2.0 Hz, 2H), 3.18-3.02 (m, 4H), 2.84 (br s, 3H), 2.76-2.67 (m, 1H), 2.63-2.56 (m, 1H), 2.38- 2.28 (m, 1H), 2.18-2.09 (m, 1H). MS: (ESI) m/z calculated for C₂₆H₂₇N₇O₃ [M + H]⁺ 486.2, found 486.2. |
| 198 | 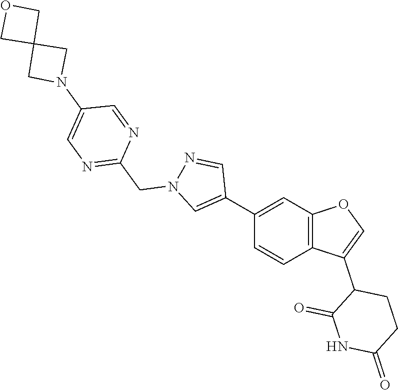 [SMILES: O=C1CCC(c2coc3cc(-c4cnn(Cc5ncc(N6CC7(COC7)C6)cn5)c4)ccc23)C(=O)N1] | ¹H NMR (400 MHz, DMSO-d₆) δ 10.91 (s, 1 H), 8.29 (s, 1 H), 8.03 (s, 2 H), 7.93 (s, 1 H), 7.86 (s, 1 H), 7.80 (s, 1 H), 7.55 (d, J = 8.0 Hz, 1 H), 7.49 (d, J = 8.0 Hz, 1 H), 5.39 (s, 2 H), 4.70 (s, 4 H), 4.20-4.02 (m, 5 H), 2.83-2.64 (m, 2 H), 2.63- 2.55 (m, 2 H), 2.40-2.27 (m, 2 H), 2.22-2.04 (m, 2 H), 1.24 (s, 1 H). |
| 199 | 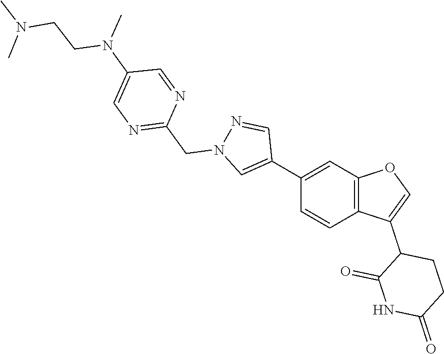 [SMILES: CN(C)CCN(C)c1cnc(Cn2cc(-c3ccc4c(C5CCC(=O)NC5=O)coc4c3)cn2)nc1] | ¹H NMR (400 MHz, DMSO-d₆) δ 10.92 (s, 1H), 8.30-8.21 (m, 3H), 7.99-7.78 (m, 3H), 7.57- 7.53 (m, 1H), 7.52-7.47 (m, 1H), 5.38 (s, 2H), 4.13 (dd, J = 4.8, 12.0 Hz, 1H), 3.50 (t, J = 8.0 Hz, 2H), 2.94 (s, 3H), 2.74 (d, J = 8.0, 12.0 Hz, 1H), 2.59 (d, J = 4.0, 16.0 Hz, 1H), 2.54-2.51 (m, 2H), 2.33 (dd, J = 4.0, 12.0 Hz, 1H), 2.27 (s, 6H), 2.18-2.08 (m, 1H). MS: (ESI) m/z calculated for C₂₆H₂₉N₇O₃ [M + H]⁺ 488.2, found 488.3. |
| 200 | 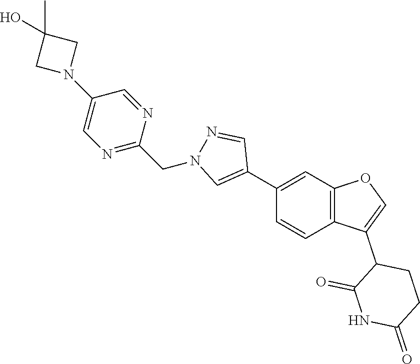 [SMILES: CC1(O)CN(c2cnc(Cn3cc(-c4ccc5c(C6CCC(=O)NC6=O)coc5c4)cn3)nc2)C1] | ¹H NMR (400 MHz, DMSO-d₆) δ 10.90 (s, 1H), 8.28 (s, 1H), 8.03 (s, 2H), 7.93 (s, 1H), 7.86 (s, 1H), 7.80 (s, 1H), 7.58-7.39 (m, 2H), 5.61 (s, 1H), 5.39 (s, 2H), 4.13 (dd, J = 4.4, 11.6 Hz, 1H), 3.90-3.70 (m, 4H), 2.67 (d, J = 1.6 Hz, 2H), 2.33 (br d, J = 1.6 Hz, 2H), 1.43 (s, 3H). MS: (ESI) m/z calculated for C₂₅H₂₄N₆O₄ [M + H]⁺ 473.1, found 473.1. |
| 201 | 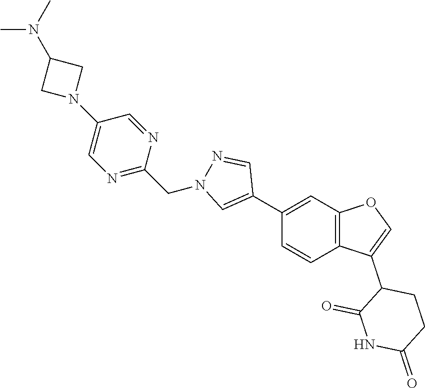 [SMILES: CN(C)C1CN(c2cnc(Cn3cc(-c4ccc5c(C6CCC(=O)NC6=O)coc5c4)cn3)nc2)C1] | ¹H NMR (400 MHz, DMSO-d₆) δ 10.89 (br s, 1H), 8.28 (s, 1H), 8.01 (s, 2H), 7.93 (s, 1H), 7.85 (s, 1H), 7.80 (s, 1H), 7.57-7.53 (m, 1H), 7.51- 7.45 (m, 1H), 5.39 (s, 2H), 4.13 (dd, J = 4.8, 12.0 Hz, 1H), 3.99 (t, J = 8.4 Hz, 2H), 3.69 (dd, J = 8.0, 8.60Hz, 2H), 3.28-3.20 (m, 1H), 2.81-2.68 (m, 1H), 2.64-2.54 (m, 1H), 2.38-2.26 (m, 1H), 2.13 (dd, J = 3.6, 8.4 Hz, 1H), 2.09 (s, 6H). MS: (ESI) m/z calculated for C₂₆H₂₇N₇O₃ [M + H]⁺ 486.2, found 486.3. |
| 202 | 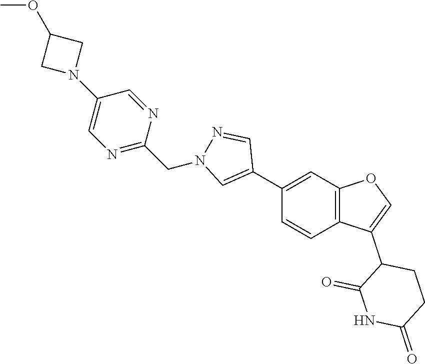 [SMILES: COC1CN(c2cnc(Cn3cc(-c4ccc5c(C6CCC(=O)NC6=O)coc5c4)cn3)nc2)C1] | ¹H NMR (400 MHz, DMSO-d₆) δ 10.91 (s, 1H), 8.29 (s, 1H), 8.03 (s, 2H), 7.93 (s, 1H), 7.86 (s, 1H), 7.80 (s, 1H), 7.57-7.52 (m, 1H), 7.51-7.47 (m, 1H), 5.44-5.34 (m, 2H), 4.38-4.31 (m, 1H), 4.18-4.09 (m, 3H), 3.75-3.72 (m, 2H), 3.23 (s, 3H), 2.78-2.70 (m, 1H), 2.61-2.54 (m, 1H), 2.38-2.27 (m, 1H), 2.17-2.07 (m, 1H). MS: (ESI) m/z calculated for C₂₅H₂₄N₆O₄ [M + H]⁺ 473.1, found 473.2. |
| 203 | 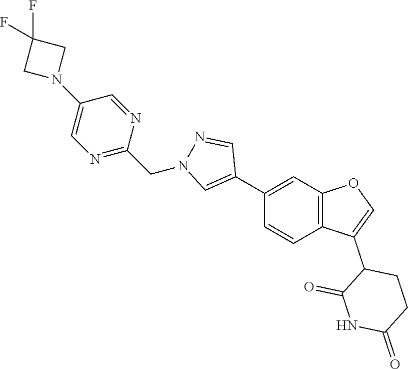 [SMILES: O=C1CCC(c2coc3cc(-c4cnn(Cc5ncc(N6CC(F)(F)C6)cn5)c4)ccc23)C(=O)N1] | ¹H NMR (400 MHz, DMSO-d₆) δ 10.91 (s, 1 H), 8.39 (s, 1 H), 8.30 (s, 1 H), 8.17 (s, 2 H), 7.95- 7.92 (m, 1 H), 7.86 (s, 1 H), 7.81-7.78 (m, 1 H), 7.57-7.53 (m, 1 H), 7.51-7.47 (m, 1 H), 5.45- 5.41 (m, 2 H), 4.46-4.38 (m, 4 H), 4.16-4.10 (m, 1 H), 2.79-2.73 (m, 1 H), 2.72-2.66 (m, 1 H), 2.35-2.31 (m, 1 H), 2.16-2.10 (m, 1 H). MS: (ESI) m/z calculated for C₂₄H₂₀F₂N₆O₃ [M + H]⁺ 479.1 found 479.0. |
| 204 | 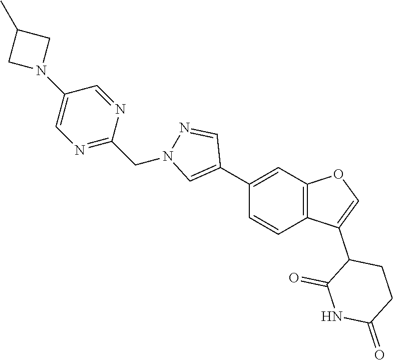 [SMILES: CC1CN(c2cnc(Cn3cc(-c4ccc5c(C6CCC(=O)NC6=O)coc5c4)cn3)nc2)C1] | ¹H NMR (400 MHz, DMSO-d₆) δ 10.89 (s, 1H), 8.28 (s, 1H), 7.98 (s, 2H), 7.94-7.91 (m, 1H), 7.85 (s, 1H), 7.81-7.78 (m, 1H), 7.56-7.53 (m, 1H), 7.50-7.47 (m, 1H), 5.38 (s, 2H), 4.13 (dd, J = 5.2, 12.0 Hz, 1H), 4.05 (t, J = 7.6 Hz, 2H), 3.51- 3.47 (m, 2H), 2.88-2.81 (m, 1H), 2.63-2.54 (m, 1H), 2.35-2.30 (m, 1H), 2.17-2.09 (m, 1H), 1.26-1.19 (m, 4H). MS: (ESI) m/z calculated for C₂₅H₂₄N₆O₃ [M + H]⁺ 457.1., found 457.1. |
| 205 | 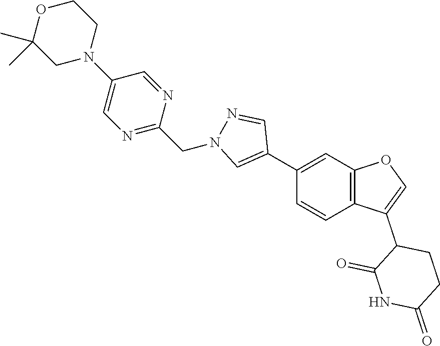 [SMILES: CC1(C)CN(c2cnc(Cn3cc(-c4ccc5c(C6CCC(=O)NC6=O)coc5c4)cn3)nc2)CCO1] | ¹H NMR (400 MHz, DMSO-d₆) δ 10.90 (s, 1H), 8.48-8.41 (m, 2H), 8.32-8.26 (m, 1H), 7.97- 7.77 (m, 3H), 7.59-7.44 (m, 2H), 5.45-5.35 (m, 2H), 4.17-4.08 (m, 1H), 3.79-3.71 (m, 2H), 3.20-3.16 (m, 2H), 3.08 (s, 2H), 2.78-2.70 (m, 1H), 2.60 (d, J = 4.0 Hz, 1H), 2.33 (br s, 1H), 2.15 (d, J = 4.8 Hz, 1H), 1.21 (s, 6H). MS: (ESI) m/z calculated for C₂₇H₂₈N₆O₄ [M + H]⁺ 501.2, found 501.2. |
| 206 | 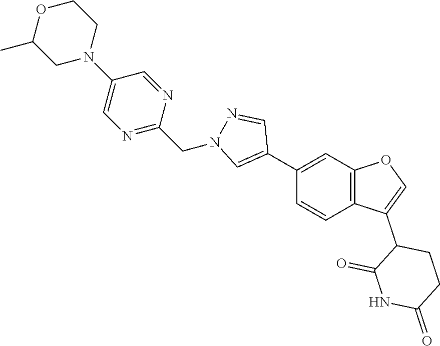 [SMILES: CC1CN(c2cnc(Cn3cc(-c4ccc5c(C6CCC(=O)NC6=O)coc5c4)cn3)nc2)CCO1] | ¹H NMR (400 MHz, DMSO-d₆) δ 10.90 (s, 1 H), 8.47 (s, 2 H), 8.32-8.28 (m, 1 H), 7.94 (s, 1 H), 7.88-7.84 (m, 1 H), 7.82-7.78 (m, 1 H), 7.56- 7.47 (m, 2 H), 5.43-5.40 (m, 2 H), 4.16-4.10 (m, 1 H), 3.94-3.88 (m, 1 H), 3.72 (d, J = 11.6 Hz, 1 H), 3.68-3.58 (m, 4 H), 2.80-2.74 (m, 1 H), 2.74-2.69 (m, 1 H), 2.42-2.38 (m, 1 H), 2.37-2.26 (m, 1 H), 2.18-2.06 (m, 1 H), 1.15- 1.13 (m, 3 H). MS: (ESI) m/z calculated for C₂₆H₂₆N₆O₄ [M + H]⁺ 487.2, found 487.1. |
| 207 | 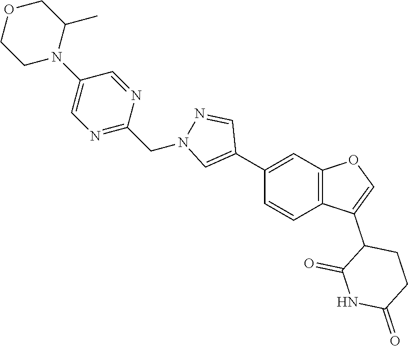 [SMILES: CC1COCCN1c1cnc(Cn2cc(-c3ccc4c(C5CCC(=O)NC5=O)coc4c3)cn2)nc1] | ¹H NMR (400 MHz, DMSO-d₆) δ 8.42 (s, 2H), 8.31 (s, 1H), 7.95 (s, 1H), 7.86 (s, 1H), 7.81 (s, 1H), 7.57-7.53 (m, 1H), 7.51 (d, J = 1.2 Hz, 1H), 5.42 (s, 2H), 4.13 (br d, J = 6.4 Hz, 1H), 4.03-3.89 (m, 2H), 3.70 (d, J = 2.0 Hz, 2H), 3.55 (br d, J = 3.6 Hz, 1H), 3.38 (br s, 1H), 3.30 (br s, 1H), 3.10-3.00 (m, 1H), 2.80-2.70 (m, 1H), 2.61 (br s, 1H), 2.36-2.31 (m, 1H), 2.18- 2.09 (m, 1H), 1.05 (d, J = 6.4 Hz, 3H). MS: (ESI) m/z calculated for C₂₆H₂₆N₆O₄ [M + H]⁺ 487.2, found 487.2. |
| 208 | 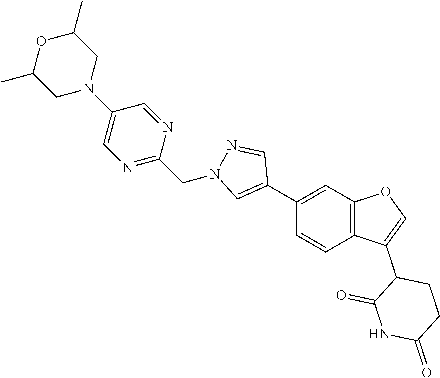 [SMILES: CC1CN(c2cnc(Cn3cc(-c4ccc5c(C6CCC(=O)NC6=O)coc5c4)cn3)nc2)CC(C)O1] | ¹H NMR (400 MHz, DMSO-d₆) δ 10.94-10.87 (m, 1H), 8.47 (s, 2H), 8.33-8.24 (m, 1H), 7.94 (s, 1H), 7.86 (s, 1H), 7.80 (s, 1H), 7.57-7.53 (m, 1H), 7.51-7.47 (m, 1H), 5.45-5.39 (m, 2H), 4.16-4.10 (m, 1H), 3.75-3.65 (m, 4H), 2.69- 2.64 (m, 1H), 2.60 (br t, J = 4.0 Hz, 1H), 2.37- 2.31 (m, 3H), 2.17-2.09 (m, 1H), 1.14 (d, J = 6.0 Hz, 6H). MS: (ESI) m/z calculated for C₂₇H₂₈N₆O₄ [M + H]⁺ 501.2, found 501.3. |
| 209 | 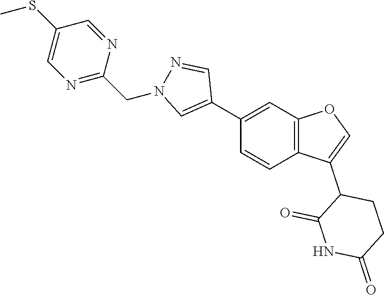 [SMILES: CSc1cnc(Cn2cc(-c3ccc4c(C5CCC(=O)NC5=O)coc4c3)cn2)nc1] | ¹H NMR (400 MHz, DMSO-d₆) δ 10.90 (s, 1H), 8.73 (s, 2H), 8.34 (s, 1H), 7.97 (s, 1H), 7.86 (s, 1H), 7.81 (s, 1H), 7.58-7.53 (m, 1H), 7.52-7.47 (m, 1H), 5.52 (s, 2H), 4.17-4.10 (m, 1H), 2.80- 2.70 (m, 1H), 2.60 (m, 1H), 2.56 (s, 3H), 2.33 (m, 1H), 2.13 (m, 1H). MS: (ESI) m/z calculated for C₂₂H₁₉N₅O₃S [M + H]⁺ 434.1, found 434.1. |
| 210 | 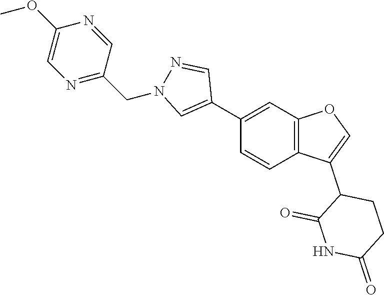 [SMILES: COc1cnc(Cn2cc(-c3ccc4c(C5CCC(=O)NC5=O)coc4c3)cn2)cn1] | ¹H NMR (400 MHz, DMSO-d₆) δ 10.90 (s, 1H), 8.33 (s, 1H), 8.29 (d, J = 1.2 Hz, 1H), 8.23 (s, 1H), 7.98 (s, 1H), 7.85 (s, 1H), 7.80 (s, 1H), 7.64- 7.35 (m, 2H), 5.42 (s, 2H), 4.12 (dd, J = 4.8, 12.0 Hz, 1H), 3.90 (s, 3H), 2.82-2.68 (m, 1H), 2.64-2.55 (m, 1H), 2.32 (dq, J = 4.4, 12.4 Hz, 1H), 2.12 (qd, J = 4.4, 13.2 Hz, 1H). MS: (ESI) m/z calculated for C₂₂H₁₉N₅O₄ [M + H]⁺ 418.1, found 418.0. |
| 211 | 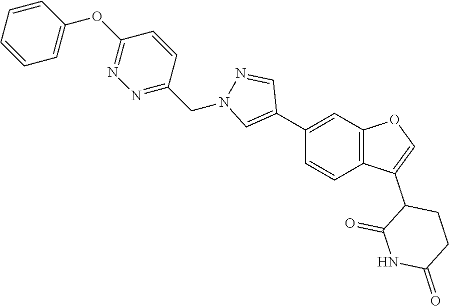 [SMILES: O=C1CCC(c2coc3cc(-c4cnn(Cc5ccc(Oc6ccccc6)nn5)c4)ccc23)C(=O)N1] | ¹H NMR (400 MHz, DMSO-d₆) δ 10.95-10.88 (m, 1H), 8.41 (s, 1H), 8.04 (s, 1H), 7.87 (s, 1H), 7.82 (s, 1H), 7.63 (d, J = 9.2 Hz, 1H), 7.59-7.54 (m, 1H), 7.53-7.42 (m, 4H), 7.33-7.17 (m, 3H), 5.62 (s, 2H), 4.13 (dd, J = 4.8, 12.0 Hz, 1H), 2.80- 2.69 (m, 1H), 2.62-2.54 (m, 1H), 2.33 (d, J = 4.4, 12.4 Hz, 1H), 2.18-2.09 (m, 1H). MS: (ESI) m/z calculated for C₂₇H₂₁N₅O₄ [M + H]⁺ 480.1, found 480.0. |
| 212 | 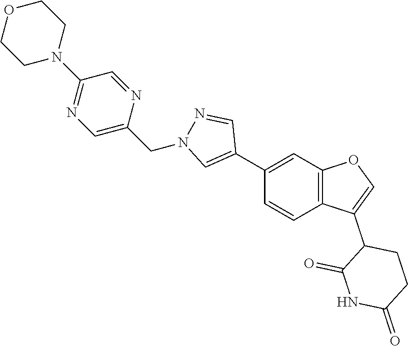 [SMILES: O=C1CCC(c2coc3cc(-c4cnn(Cc5cnc(N6CCOCC6)cn5)c4)ccc23)C(=O)N1] | ¹HNMR (400 MHz, DMSO-d₆) δ 10.89 (s, 1H), 8.29 (s, 1H), 8.28 (d, J = 1.2 Hz, 1H), 8.14 (d, J = 1.2 Hz, 1H), 7.95 (s, 1H), 7.85 (s, 1H), 7.79 (s, 1H), 7.56-7.52 (m, 1H), 7.50-7.46 (m, 1H), 5.31 (s, 2H), 4.14-4.10 (m, 1H), 3.71-3.68 (m, 4H), 3.53-3.50 (m, 4H), 2.79-2.70 (m, 1H), 2.58 (m, 1H), 2.38-2.27 (m, 1H), 2.16-2.07 (m, 1H). MS: (ESI) m/z calculated for C₂₅H₂₄N₆O₄ [M + H]⁺ 473.1, found 473.1. |
| 213 | 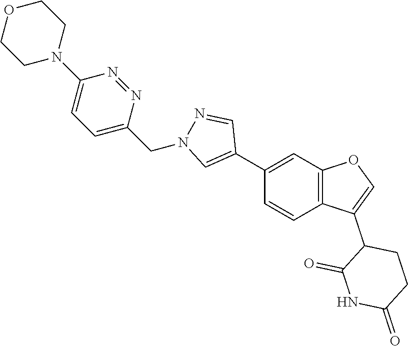 [SMILES: O=C1CCC(c2coc3cc(-c4cnn(Cc5ccc(N6CCOCC6)nn5)c4)ccc23)C(=O)N1] | ¹HNMR (400 MHz, DMSO-d₆) δ 10.91 (s, 1H), 8.36 (s, 1H), 8.00 (s, 1H), 7.90-7.77 (m, 2H), 7.59-7.45 (m, 2H), 7.37-7.24 (m, 2H), 5.48 (s, 2H), 4.16-4.07 (m, 1H), 3.76-3.68 (m, 4H), 3.56-3.49 (m, 4H), 2.74 (s, 1H), 2.62-2.54 (m, 1H), 2.33 (br dd, J = 4.4, 12.4 Hz, 1H), 2.18- 2.07 (m, 1H). MS: (ESI) m/z calculated for C₂₅H₂₄N₆O₄ [M + H]⁺ 473.1, found 473.1. |
| 214 | 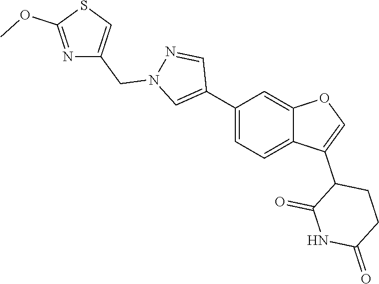 [SMILES: COc1nc(Cn2cc(-c3ccc4c(C5CCC(=O)NC5=O)coc4c3)cn2)cs1] | ¹H NMR (400 MHz, DMSO-d₆) δ 10.90 (s, 1H), 8.28 (s, 1H), 7.99 (s, 1H), 7.86 (s, 1H), 7.81 (s, 1H), 7.57-7.53 (m, 1H), 7.51-7.47 (m, 1H), 6.84 (s, 1H), 5.24 (s, 2H), 4.13 (m, 1H), 4.00- 3.98 (m, 3H), 2.79-2.69 (m, 1H), 2.58 (m, 1H), 2.39-2.27 (m, 1H), 2.13 (m, 1H). MS: (ESI) m/z calculated for C₂₁H₁₈N₄O₄S [M + H]⁺ 423.1, found 422.9. |
| 215 | 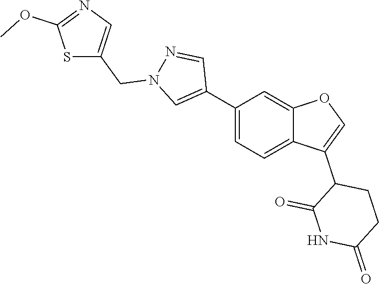 [SMILES: COc1ncc(Cn2cc(-c3ccc4c(C5CCC(=O)NC5=O)coc4c3)cn2)s1] | ¹H NMR (400 MHz, DMSO-d₆) δ 10.90 (s, 1H), 8.29 (s, 1H), 8.00 (s, 1H), 7.86 (s, 1H), 7.79 (s, 1H), 7.58-7.52 (m, 1H), 7.50-7.44 (m, 1H), 7.25 (s, 1H), 5.44 (s, 2H), 4.13 (m, 1H), 3.98 (s, 3H), 2.79-2.69 (m, 1H), 2.61-2.54 (m, 1H), 2.33 (m, 1H), 2.16-2.08 (m, 1H). MS: (ESI) m/z calculated for C₂₁H₁₈N₄O₄S [M + H]⁺ 423.1, found 423.1. |
| 216 | 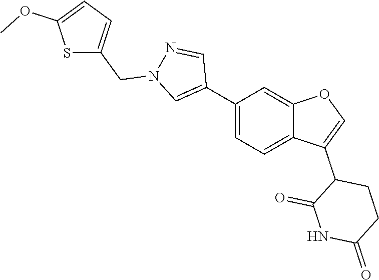 [SMILES: COc1ccc(Cn2cc(-c3ccc4c(C5CCC(=O)NC5=O)coc4c3)cn2)s1] | ¹H NMR (400 MHz, DMSO-d₆) δ 10.90 (s, 1H), 8.26 (s, 1H), 7.97 (s, 1H), 7.86 (s, 1H), 7.79 (s, 1H), 7.57-7.51 (m, 1H), 7.49-7.44 (m, 1H), 6.81 (d, J = 4.0 Hz, 1H), 6.17 (d, J = 3.6 Hz, 1H), 5.35 (s, 2H), 4.12 (dd, J = 4.8, 11.9 Hz, 1H), 3.81 (s, 3H), 2.80-2.68 (m, 1H), 2.58 (td, J = 3.6, 17.3 Hz, 1H), 2.32 (dq, J = 4.4, 12.4 Hz, 1H), 2.18-2.07 (m, 1H). MS: (ESI) m/z calculated for C₂₂H₁₉N₃O₄S [M + H]⁺ 422.1, found 422.0. |
| 217 | 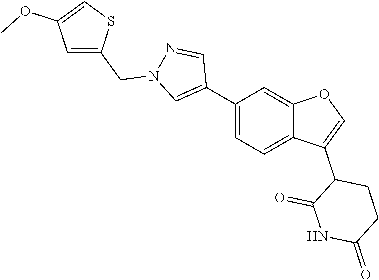 [SMILES: COc1csc(Cn2cc(-c3ccc4c(C5CCC(=O)NC5=O)coc4c3)cn2)c1] | ¹HNMR (400 MHz, DMSO-d₆) δ 10.90 (br s, 1H), 8.30 (s, 1H), 7.99 (s, 1H), 7.88-7.76 (m, 2H), 7.57-7.52 (m, 1H), 7.49-7.45 (m, 1H), 6.82 (s, 1H), 6.49 (d, J = 1.6 Hz, 1H), 5.42 (s, 2H), 4.13 (m, 1H), 3.70 (s, 3H), 2.74 (m, 1H), 2.60-2.55 (m, 1H), 2.38-2.26 (m, 1H), 2.17- 2.06 (m, 1H). MS: (ESI) m/z calculated for C₂₂H₁₉N₃O₄S [M + H]⁺ 422.1, found 422.0. |
| 301 | 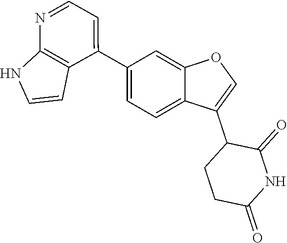 [SMILES: O=C1CCC(c2coc3cc(-c4ccnc5[nH]ccc45)ccc23)C(=O)N1] | ¹H NMR (400 MHz, DMSO-d₆) δ 11.81 (brs, 1H), 10.94 (s, 1H), 8.29 (d, J = 4.8 Hz, 1H), 8.01 (s, 1H), 7.95 (d, J = 0.8, 1H), 7.75 (d, J = 8.4, 1H), 7.67 (dd, J = 8.4, 1.6 Hz, 1H), 7.55 (t, J = 2.8 Hz, 1H), 7.25 (d, J = 4.8 Hz, 1H), 6.66 (dd, J = 3.2, 1.6 Hz, 1H), 4.20 (dd, J = 12.0, 4.8 Hz, 1H), 2.72-2.84 (m, 1H), 2.58-2.68 (m, 1H), 2.34- 2.45 (m, 1H), 2.12-2.21 (m, 1H). LCMS: calc. for C₂₀H₁₅N₃O₃: 345.1, found: [M + H]⁺ 346.1 |
| 302 | 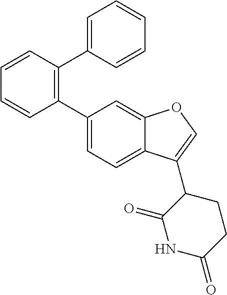 [SMILES: O=C1CCC(c2coc3cc(-c4ccccc4-c4ccccc4)ccc23)C(=O)N1] | ¹H NMR (400 MHz, DMSO-d₆) δ 10.87 (s, 1H), 7.85 (s, 1H), 7.45-7.49 (m, 3H), 7.40-7.45 (m, 2H), 7.29 (s, 1H), 7.19-7.27 (m, 3H), 7.11-7.16 (m, 2H), 6.98 (dd, J = 8.0, 1.2 Hz, 1H), 4.10 (dd, J = 12.0, 4.8 Hz, 1H), 2.66-2.78 (m, 1H), 2.54- 2.61 (m, 1H), 2.22-2.35 (m, 1H), 2.04-2.14 (m, 1H). LCMS: calc. for C₂₅H₁₉NO₃: 381.1, found: [M + H]⁺ 382.2. |
| 303 | 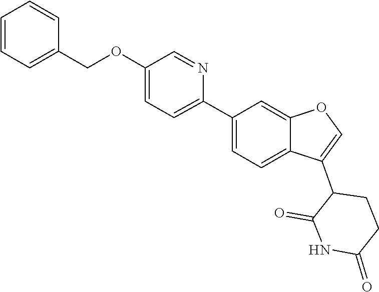 [SMILES: O=C1CCC(c2coc3cc(-c4ccc(OCc5ccccc5)cn4)ccc23)C(=O)N1] | ¹H NMR (400 MHz, CDCl₃) δ 8.50 (s, 1H), 8.00- 8.14 (m, 2H), 7.70-7.92 (m, 2H), 7.63 (s, 1H), 7.57 (d, J = 8.0 Hz, 1H), 7.34-7.50 (m, 6H), 5.19 (s, 2H), 4.04 (t, J = 7.6 Hz, 1H), 2.69-2.88 (m, 2H), 2.36-2.47 (m, 2H). LCMS: calc. for C₂₅H₂₀N₂O₄: 412.1, found: [M + H]⁺ 413.1. |
| 304 | 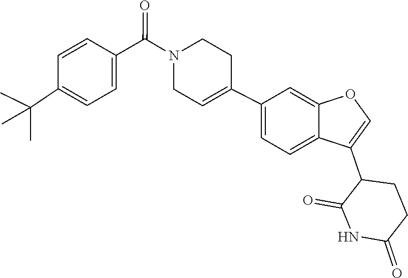 [SMILES: CC(C)(C)c1ccc(C(=O)N2CC=C(c3ccc4c(C5CCC(=O)NC5=O)coc4c3)CC2)cc1] | ¹H NMR (400 MHz, DMSO-d₆) δ 10.89 (s, 1H), 7.89 (s, 1H), 7.63 (s, 1H), 7.54 (d, J = 8.0 Hz, 1H), 7.49 (d, J = 7.2 Hz, 2H), 7.40 (d, J = 7.6 Hz, 3H), 6.08-6.41 (m, 1H), 4.02-4.42 (m, 3H), 3.79-3.93 (m, 1H), 3.50-3.71 (m, 2H), 2.58-2.86 (m, 2H), 2.23-2.39 (m, 2H), 2.06-2.19 (m, 1H), 1.31 (s, 9H). LCMS: calc. for C₂₉H₃₀N₂O₄: 470.2, found: [M + H]⁺ 471.3. |
| 305 | 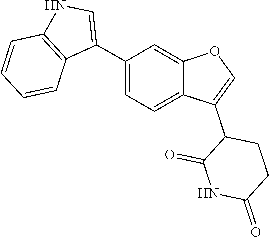 [SMILES: O=C1CCC(c2coc3cc(-c4c[nH]c5ccccc45)ccc23)C(=O)N1] | ¹H NMR (400 MHz, DMSO-d₆) δ 11.39 (s, 1H), 10.91 (s, 1H), 7.87-7.92 (m, 2H), 7.82 (s, 1H), 7.74 (d, J = 2.8 Hz, 1H), 7.57-7.64 (m, 2H), 7.46 (d, J = 7.6 Hz, 1H), 7.09-7.19 (m, 2H), 4.16 (dd, J = 11.6, 4.8 Hz, 1H), 2.71-2.82 (m, 1H), 2.56- 2.63 (m, 1H), 2.32-2.41 (m, 1H), 2.13-2.19 (m, 1H). LCMS: calc. for C₂₁H₁₆N₂O₃: 344.1, found: [M + H]⁺ 345.1. |
| 306 | 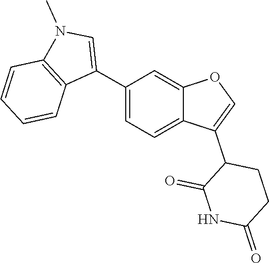 [SMILES: Cn1cc(-c2ccc3c(C4CCC(=O)NC4=O)coc3c2)c2ccccc21] | ¹H NMR (400 MHz, DMSO-d₆) δ 10.92 (s, 1H), 7.93 (d, J = 7.6 Hz, 1H), 7.89 (s, 1H), 7.80 (s, 1H), 7.75 (s, 1H), 7.61-7.65 (m, 1H), 7.56-7.59 (m, 1H), 7.52 (d, J = 8.4 Hz, 1H), 7.21-7.27 (m, 1H), 7.13-7.18 (m, 1H), 4.16 (dd, J = 12.0, 4.8 Hz, 1H), 3.86 (s, 3H), 2.71-2.82 (m, 1H), 2.57- 2.59 (m, 1H), 2.31-2.36 (m, 1H), 2.11-2.20 (m, 1H). LCMS: calc. for C₂₂H₁₈N₂O₃: 358.1, found: [M + H]⁺ 359.1 |
| 307 | 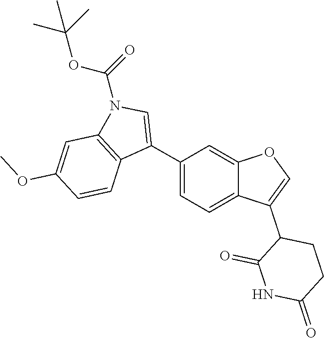 [SMILES: COc1ccc2c(-c3ccc4c(C5CCC(=O)NC5=O)coc4c3)cn(C(=O)OC(C)(C)C)c2c1] | ¹H NMR (400 MHz, DMSO-d₆) δ 10.93 (s, 1H), 7.96 (s, 1H), 7.88 (s, 1H), 7.83 (s, 1H), 7.79 (d, J = 8.8 Hz, 1H), 7.74 (d, J = 2.4 Hz, 1H), 7.69 (d, J = 8.0 Hz, 1H), 7.59 (dd, J = 8.0, 1.2 Hz, 1H), 6.99 (dd, J = 8.8, 2.4 Hz, 1H), 4.19 (dd, J = 12.0, 4.8 Hz, 1H), 3.85 (s, 3H), 2.73-2.83 (m, 1H), 2.57 (m, 1H), 2.40 (m, 1H), 2.13-2.20 (m, 1H), 1.67 (s, 9H). LCMS: calc. for C₂₇H₂₆N₂O₆: 474.2, found: [M + H]⁺ 475.2 |
| 308 | 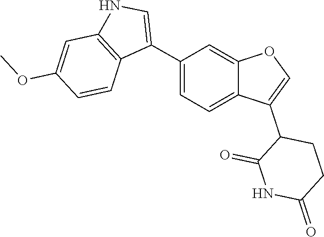 [SMILES: COc1ccc2c(-c3ccc4c(C5CCC(=O)NC5=O)coc4c3)c[nH]c2c1] | ¹H NMR (400 MHz, DMSO-d₆) δ 11.17 (s, 1H), 10.92 (br s, 1H), 7.87 (s, 1H), 7.75-7.79 (m, 2H), 7.56-7.62 (m, 3H), 6.94 (d, J = 2.4 Hz, 1H), 6.76 (dd, J = 8.8, 2.4 Hz, 1H), 4.15 (dd, J = 11.6, 4.4 Hz, 1H), 3.79 (s, 3H), 2.70-2.83 (m, 1H), 2.55- 2.59 (m, 1H), 2.35-2.44 (m, 1H), 2.13-2.20 (m, 1H). LCMS: calc. for C₂₂H₁₈N₂O₄: 374.1, found: [M + H]⁺ 375.1 |
| 309 | 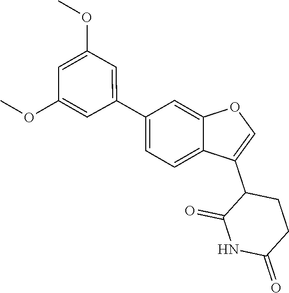 [SMILES: COc1cc(OC)cc(-c2ccc3c(C4CCC(=O)NC4=O)coc3c2)c1] | ¹H NMR (400 MHz, DMSO-d₆) δ 10.92 (s, 1H), 7.94 (s, 1H), 7.88 (s, 1H), 7.60 (dd, J = 9.2, 4.8 Hz, 2H), 6.85 (s, 2H), 6.51 (s, 1H), 4.17 (dd, J = 11.6, 4.0 Hz, 1H), 3.82 (s, 6H), 2.72-2.80 (m, 1H), 2.57-2.61 (m, 1H), 2.31-2.39 (m, 1H), 2.08- 2.24 (m, 1H). LCMS: calc. for C₂₁H₁₉NO₅: 365.1, found: [M + H]⁺ 366.1 |
| 310 | 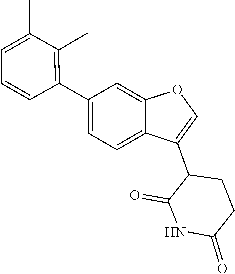 [SMILES: Cc1cccc(-c2ccc3c(C4CCC(=O)NC4=O)coc3c2)c1C] | ¹H NMR (400 MHz, DMSO-d₆) δ 10.93 (s, 1H), 7.94 (s, 1H), 7.62 (d, J = 8.0 Hz, 1H), 7.47 (s, 1H), 7.10-7.22 (m, 3H), 7.07 (d, J = 6.8 Hz, 1H), 4.17 (dd, J = 12.0, 4.8 Hz, 1H), 3.41-3.49 (m, 1H), 2.70-2.82 (m, 1H), 2.57-2.62 (m, 1H), 2.33- 2.42 (m, 1H), 2.30 (s, 3H), 2.12 (s, 3H). LCMS: calc. for C₂₁H₁₉NO₃: 333.1, found: [M + H]⁺ 334.1 |
| 311 | 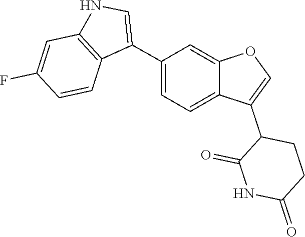 [SMILES: O=C1CCC(c2coc3cc(-c4c[nH]c5cc(F)ccc45)ccc23)C(=O)N1] | ¹H NMR (400 MHz, DMSO-d₆) δ 11.45 (s, 1H), 10.92 (s, 1H), 7.85-7.92 (m, 2H), 7.81 (s, 1H), 7.73 (d, J = 2.4 Hz, 1H), 7.52-7.68 (m, 2H), 7.23 (dd, J = 10.0, 2.4 Hz, 1H), 6.92-7.01 (m, 1H), 4.16 (dd, J = 12.4, 5.2 Hz, 1H), 2.71-2.81 (m, 1H), 2.59-2.64 (m, 1H), 2.35-2.39 (m, 1H), 2.09- 2.18 (m, 1H). LCMS: calc. for: C₂₁H₁₅FN₂O₃: 362.1, found: [M + H]⁺ 363.1 |
| 312 | 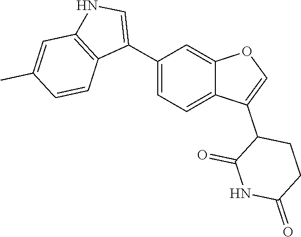 [SMILES: Cc1ccc2c(-c3ccc4c(C5CCC(=O)NC5=O)coc4c3)c[nH]c2c1] | 1H NMR (400 MHz, DMSO-d₆) δ 11.23 (s, 1H), 10.93 (s, 1H), 7.87 (s, 1H), 7.77-7.81 (m, 2H), 7.66 (d, J = 2.4 Hz, 1H), 7.55-7.62 (m, 2H), 7.24 (s, 1H), 6.94 (dd, J = 8.4, 1.2 Hz, 1H), 4.15 (dd, J = 12.0, 4.8 Hz, 1H), 2.72-2.81 (m, 1H), 2.56-2.63 (m, 1H), 2.42 (s, 3H), 2.31-2.37 (m, 1H), 2.11- 2.19 (m, 1H). LCMS: calc. for C₂₂H₁₈N₂O₃: 358.1, found: [M + H]⁺ 359.1 |
| 313 | 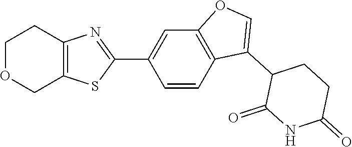 [SMILES: O=C1CCC(c2coc3cc(-c4nc5c(s4)COCC5)ccc23)C(=O)N1] | ¹H NMR (400 MHz, DMSO-d₆) δ 10.93 (s, 1H), 8.08 (d, J = 0.8 Hz, 1H), 8.03 (s, 1H), 7.80 (dd, J = 8.4, 1.2 Hz, 1H), 7.69 (d, J = 8.0 Hz, 1H), 4.83 (s, 2H), 4.19 (dd, J = 12.0, 4.8 Hz, 1H), 3.98 (t, J = 5.6 Hz, 2H), 2.85-2.92 (m, 2H), 2.71-2.82 (m, 1H), 2.60-2.64 (m, 1H), 2.35-2.41 (m, 1H), 2.10- 2.18 (m, 1H). LCMS: calc. for C₁₉H₁₆N₂O₄S: 368.1, found: [M + H]⁺ 369.2 |
| 314 | 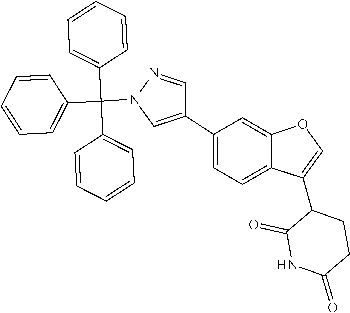 [SMILES: O=C1CCC(c2coc3cc(-c4cnn(C(c5ccccc5)(c5ccccc5)c5ccccc5)c4)ccc23)C(=O)N1] | ¹H NMR (400 MHz, DMSO-d₆) δ 10.89 (s, 1H), 8.15 (s, 1H), 7.90 (s, 1H), 7.85 (s, 1H), 7.81 (d, J = 0.8 Hz, 1H), 7.49-7.55 (m, 1H), 7.32-7.44 (m, 10H), 7.13 (dd, J = 8.0, 1.6 Hz, 6H), 4.12 (dd, J = 12.0, 4.8 Hz, 1H), 2.68-2.81 (m, 1H), 2.55-2.62 (m, 1H), 2.26-2.34 (m, 1H), 2.08-2.17 (m, 1H). LCMS: calc. for C₃₅H₂₇N₃O₃: 537.2, found: [M + H]⁺ 538.1 |
| 315 | 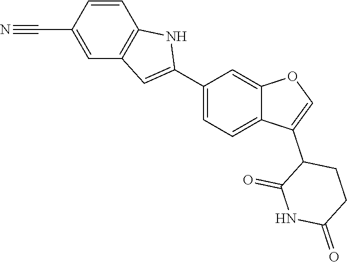 [SMILES: N#Cc1ccc2[nH]c(-c3ccc4c(C5CCC(=O)NC5=O)coc4c3)cc2c1] | ¹H NMR (400 MHz, DMSO-d₆) δ 12.19 (s, 1H), 10.93 (s, 1H), 8.13 (s, 1H), 8.07 (s, 1H), 7.99 (s, 1H), 7.82 (dd, J = 8.4, 1.2 Hz, 1H), 7.71 (d, J = 8.0 Hz, 1H), 7.56 (d, J = 8.4 Hz, 1H), 7.45 (dd, J = 8.4, 1.2 Hz, 1H), 7.14 (d, J = 1.2 Hz, 1H), 4.18 (dd, J = 12.0, 4.8 Hz, 1H), 2.72-2.79 (m, 1H), 2.35-2.40 (dd, J = 12.4, 4.0 Hz, 1H), 2.10-2.24 (m, 1H), 1.94-2.06 (m, 1H). LCMS: calc. for C₂₂H₁₅N₃O₃: 369.1, found: [M + H]⁺ 370.1. |
| 316 | 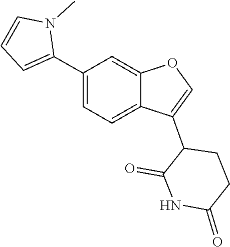 [SMILES: Cn1cccc1-c1ccc2c(C3CCC(=O)NC3=O)coc2c1] | ¹H NMR (400 MHz, DMSO-d₆) δ 10.92 (s, 1H), 7.92 (s, 1H), 7.56-7.64 (m, 2H), 7.32 (dd, J = 8.0, 1.2 Hz, 1H), 6.82-6.88 (m, 1H), 6.16-6.22 (m, 1H), 6.03-6.10 (m, 1H), 4.10-4.21 (m, 1H), 3.62- 3.73 (m, 3H), 2.70-2.82 (m, 1H), 2.55-2.63 (m, 1H), 2.28-2.42 (m, 1H), 2.08-2.19 (m, 1H). LCMS: calc. for C₁₈H₁₆N₂O₃: 308.1, found: [M + H]⁺ 309.1. |
| 317 | 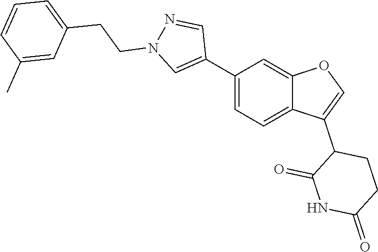 [SMILES: Cc1cccc(CCn2cc(-c3ccc4c(C5CCC(=O)NC5=O)coc4c3)cn2)c1] | ¹H NMR (400 MHz, DMSO-d₆) δ 10.90 (s, 1H), 8.15 (s, 1H), 7.96 (s, 1H), 7.85 (s, 1H), 7.75 (s, 1H), 7.54 (d, J = 8.0 Hz, 1H), 7.44 (dd, J = 8.4, 1.2 Hz, 1H), 7.13-7.18 (m, 1H), 6.96-7.03 (m, 3H), 4.33 (t, J = 7.2 Hz, 2H), 4.10-4.15 (m, 1H), 3.11 (t, J = 7.2 Hz, 2H), 2.69-2.76 (m, 1H), 2.54- 2.57 (m, 1H), 2.29-2.35 (m, 1H), 2.25 (s, 3H), 2.08-2.16 (m, 1H). LCMS: calc. for C₂₅H₂₃N₃O₃: 413.2, found: [M + H]⁺ 414.0. |
| 318 | 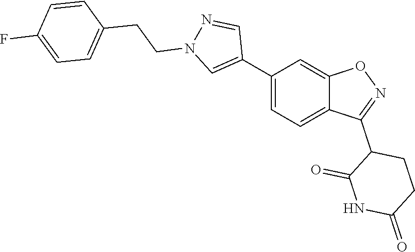 [SMILES: O=C1CCC(c2noc3cc(-c4cnn(CCc5ccc(F)cc5)c4)ccc23)C(=O)N1] | ¹H NMR (400 MHz, DMSO-d₆) δ 10.89 (s, 1H), 8.14 (s, 1H), 7.95 (s, 1H), 7.85 (s, 1H), 7.74 (s, 1H), 7.54 (d, J = 8.0 Hz, 1H), 7.43 (dd, J = 8.0, 1.6 Hz, 1H), 7.19-7.26 (m, 2H), 7.04-7.14 (m, 2H), 4.34 (t, J = 7.2 Hz, 2H), 4.09-4.15 (m, 1H), 3.15 (t, J = 7.2 Hz, 2H), 2.70-2.75 (m, 1H), 2.59- 2.61 (m, 1H), 2.29-2.35 (m, 1H), 2.08-2.17 (m, 1H). LCMS: calc. for C₂₄H₂₀FN₃O₃: 417.2, found: [M + H]⁺ 418.1. |
| 319 | 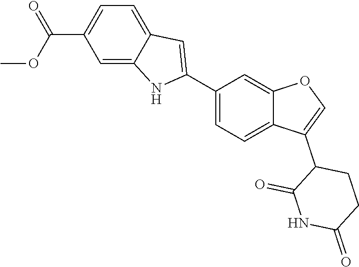 [SMILES: COC(=O)c1ccc2cc(-c3ccc4c(C5CCC(=O)NC5=O)coc4c3)[nH]c2c1] | ¹H NMR (400 MHz, DMSO-d₆) δ 11.96 (s, 1H), 10.95 (s, 1H), 8.13 (d, J = 1.2 Hz, 1H), 8.05 (d, J = 0.8 Hz, 1H), 7.99 (s, 1H), 7.83 (dd, J = 8.4, 1.6 Hz, 1H), 7.71 (d, J = 8.4 Hz, 1H), 7.59-7.65 (m, 2H), 7.11 (d, J = 1.2 Hz, 1H), 4.19 (dd, J = 12.0, 4.8 Hz, 1H), 3.87 (s, 3H), 2.72-2.82 (m, 1H), 2.57-2.61 (m, 1H), 2.35-2.38 (m, 1H), 2.11-2.19 (m, 1H). LCMS: calc. for C₂₃H₁₈N₂O₅: 402.1, found: [M + H]⁺ 403.2. |
| 320 | 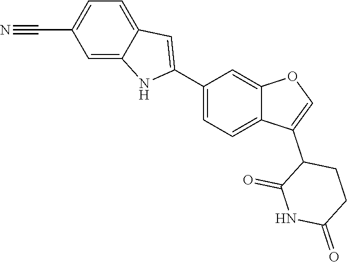 [SMILES: N#Cc1ccc2cc(-c3ccc4c(C5CCC(=O)NC5=O)coc4c3)[nH]c2c1] | ¹H NMR (400 MHz, DMSO-d₆) δ 12.19 (s, 1H), 10.94 (s, 1H), 8.17 (s, 1H), 8.00 (s, 1H), 7.82- 7.87 (m, 2H), 7.68-7.74 (m, 2H), 7.35 (dd, J = 8.4, 1.2 Hz, 1H), 7.16 (d, J = 1.2 Hz, 1H), 4.19 (dd, J = 12.0, 4.8 Hz, 1H), 2.71-2.82 (m, 1H), 2.58-2.64 (m, 1H), 2.52-2.57 (m, 1H), 2.10-2.20 (m, 1H). LCMS: calc. for C₂₂H₁₅N₃O₃: 369.1, found: [M + H]⁺ 370.1 |
| 321 | 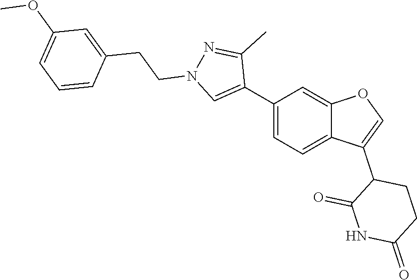 [SMILES: COc1cccc(CCn2cc(-c3ccc4c(C5CCC(=O)NC5=O)coc4c3)c(C)n2)c1] | ¹H NMR (400 MHz, DMSO-d₆) δ 10.90 (s, 1H), 7.88 (s, 2H), 7.50-7.60 (m, 2H), 7.18-7.30 (m, 2H), 6.71-6.84 (m, 3H), 4.27 (t, J = 7.2 Hz, 2H), 4.10-4.16 (m, 1H), 3.70 (s, 3H), 3.09 (t, J = 7.2 Hz, 2H), 2.70-2.81 (m, 1H), 2.54-2.63 (m, 1H), 2.37-2.46 (m, 1H), 2.35 (s, 3H), 2.09-2.16 (m, 1H). LCMS: calc. for C₂₆H₂₅N₃O₄: 443.2, found: [M + H]⁺ 444.0 |
| 322 | 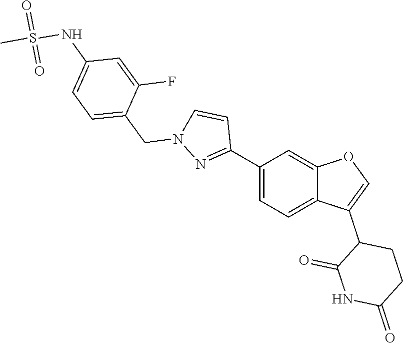 [SMILES: CS(=O)(=O)Nc1ccc(Cn2ccc(-c3ccc4c(C5CCC(=O)NC5=O)coc4c3)n2)c(F)c1] | ¹H NMR (400 MHz, DMSO-d₆) δ 10.90 (s, 1H), 10.00-10.17 (m, 1H), 7.93 (d, J = 0.8 Hz, 1H), 7.90 (s, 1H), 7.83 (d, J = 2.4 Hz, 1H), 7.71 (dd, J = 8.0, 1.2 Hz, 1H), 7.58 (d, J = 8.0 Hz, 1H), 7.28 (t, J = 8.4 Hz, 1H), 6.98-7.05 (m, 2H), 6.82 (d, J = 2.4 Hz, 1H), 5.37 (s, 2H), 4.12-4.16 (m, 1H), 3.03 (s, 3H), 2.70-2.75 (m, 1H), 2.59-2.61 (m, 1H), 2.30-2.35 (m, 1H), 2.10-2.16 (m, 1H). LCMS: calc. for C₂₄H₂₁FN₄O₅S: 496.1, found: [M + H]⁺ 496.9. |
| 323 | 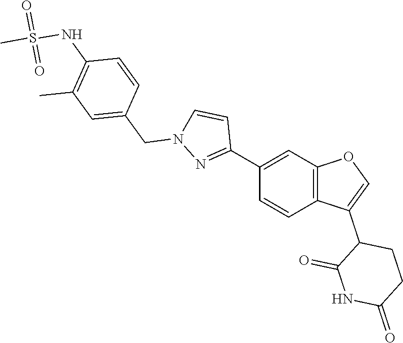 [SMILES: Cc1cc(Cn2ccc(-c3ccc4c(C5CCC(=O)NC5=O)coc4c3)n2)ccc1NS(C)(=O)=O] | ¹H NMR (400 MHz, CDCl₃) δ 8.02-8.10 (m, 1 H), 7.97 (s, 1 H), 7.77 (d, J = 8.2 Hz, 1 H), 7.60 (s, 1 H), 7.52 (d, J = 8.2 Hz, 1 H), 7.42-7.48 (m, 2 H), 7.13-7.21 (m, 2 H), 6.64 (d, J = 2.0 Hz, 1 H), 6.24 (s, 1 H), 5.34 (s, 2 H), 4.04 (t, J = 7.2 Hz, 1 H), 3.04 (s, 3 H), 2.70-2.88 (m, 2 H), 2.38- 2.45 (m, 2 H), 2.31 (s, 3 H). LCMS: calc. for C₂₅H₂₄N₄O₅S: 492.2, found: [M + H]⁺ 492.9. |
| 324 | 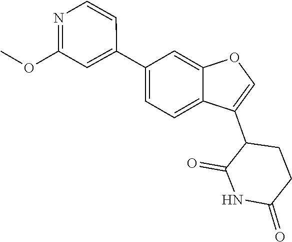 [SMILES: COc1cc(-c2ccc3c(C4CCC(=O)NC4=O)coc3c2)ccn1] | ¹H NMR (400 MHz, DMSO-d₆) δ 10.93 (s, 1H), 8.23 (d, J = 5.6 Hz, 1H), 8.05 (s, 1H), 8.01 (s, 1H), 7.70 (s, 2H), 7.40 (dd, J = 5.2, 1.2 Hz, 1H), 7.19 (s, 1H), 4.19 (dd, J = 12.0, 4.8 Hz, 1H), 3.90 (s, 3H), 2.70-2.83 (m, 1H), 2.61-2.68 (m, 1H), 2.30-2.41 (m, 1H), 2.07-2.21 (m, 1H). LCMS: calc. for C₁₉H₁₆N₂O₄: 336.1, found: [M + H]⁺ 337.1. |
| 325 | 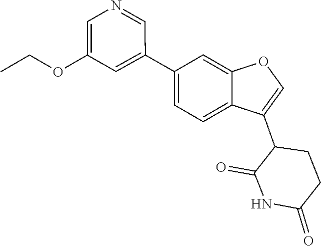 [SMILES: CCOc1cncc(-c2ccc3c(C4CCC(=O)NC4=O)coc3c2)c1] | ¹H NMR (400 MHz, DMSO-d₆) δ 10.93 (s, 1H), 8.55 (s, 1H), 8.27 (d, J = 2.4 Hz, 1H), 8.01 (s, 1H), 7.98 (s, 1H), 7.61-7.73 (m, 3H), 4.14-4.29 (m, 3H), 2.70-2.85 (m, 1H), 2.56-2.64 (m, 1H), 2.29-2.43 (m, 1H), 2.10-2.20 (m, 1H), 1.38 (t, J = 6.8 Hz, 3H). LCMS: calc. for C₂₀H₁₈N₂O₄: 350.1, found: [M + H]⁺ 351.1. |
| 326 | 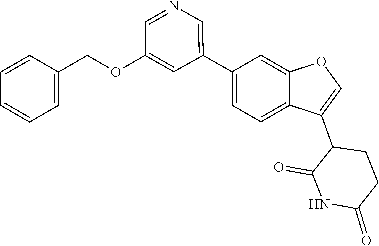 [SMILES: O=C1CCC(c2coc3cc(-c4cncc(OCc5ccccc5)c4)ccc23)C(=O)N1] | ¹H NMR (400 MHz, DMSO-d₆) δ 10.93 (s, 1H), 8.58 (s, 1H), 8.35 (s, 1H), 8.00 (d, J = 11.2 Hz, 2H), 7.81 (s, 1H), 7.61-7.74 (m, 2H), 7.51 (d, J = 7.2 Hz, 2H), 7.42 (t, J = 7.2 Hz, 2H), 7.36 (d, J = 7.2 Hz, 1H), 5.30 (s, 2H), 4.13-4.25 (m, 1H), 2.70-2.84 (m, 1H), 2.61-2.67 (m, 1H), 2.38-2.34 (m, 1H), 2.10-2.20 (m, 1H). LCMS: calc. for C₂₅H₂₀N₂O₄: 412.1, found: [M + H]⁺ 413.1. |
| 327 | 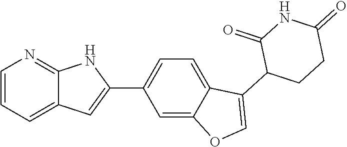 [SMILES: O=C1CCC(c2coc3cc(-c4cc5cccnc5[nH]4)ccc23)C(=O)N1] | ¹H NMR (400 MHz, DMSO-d₆) δ 12.36 (s, 1H), 10.94 (s, 1H), 8.35-8.46 (m, 1H), 8.25 (s, 1H), 8.01-8.07 (m, 2H), 7.91 (dd, J = 8.4, 1.6 Hz, 1H), 7.75 (d, J = 8.4 Hz, 1H), 7.23-7.32 (m, 2H), 4.17- 4.25 (m, 1H), 2.72-2.82 (m, 1H), 2.57-2.60 (m, 1H), 2.35-2.41 (m, 1H), 2.11-2.19 (m, 1H). LCMS: calc. for C₂₀H₁₅N₃O₃: 345.1, found: [M + H]⁺ 346.0 |
| 328 | 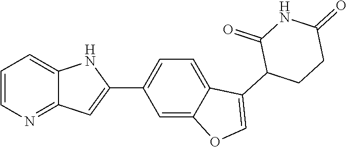 [SMILES: O=C1CCC(c2coc3cc(-c4cc5ncccc5[nH]4)ccc23)C(=O)N1] | ¹H NMR (400 MHz, DMSO-d₆) δ 11.80 (s, 1H), 10.93 (s, 1H), 8.31 (dd, J = 4.8, 1.6 Hz, 1H), 8.15-8.17 (m, 1H), 8.14 (s, 0.6H, FA), 7.98 (s, 1H), 7.85 (dd, J = 8.4, 1.2 Hz, 1H), 7.67-7.77 (m, 2H), 7.13 (d, J = 1.2 Hz, 1H), 7.09 (dd, J = 8.0, 4.8 Hz, 1H), 4.14-4.22 (m, 1H), 2.71-2.83 (m, 1H), 2.57-2.64 (m, 1H), 2.34-2.41 (m, 1H), 2.11- 2.21 (m, 1H). LCMS: calc. for C₂₀H₁₅N₃O₃: 345.1, found: [M + H]+ 345.9. |
| 329 | 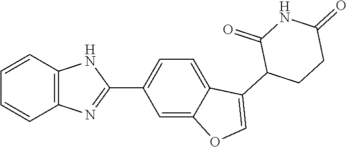 [SMILES: O=C1CCC(c2coc3cc(-c4nc5ccccc5[nH]4)ccc23)C(=O)N1] | ¹HNMR (400 MHz, d₆-DMSO) δ 12.93 (br s, 1H), 10.95 (s, 1H), 8.35 (d, J = 0.8 Hz, 1H), 8.12 (dd, J = 1.2, 8.0 Hz, 1H), 8.05 (s, 1H), 7.76 (d, J = 8.4 Hz, 1H), 7.69-7.64 (m, 1H), 7.54 (dd, J = 1.6, 8.0 Hz, 1H), 7.21 (m, 2H), 4.20 (dd, J = 8.0, 12.0 Hz, 1H), 2.75 (dd, J = 5.2, 12.0 Hz, 1H), 2.66-2.58 (m, 1H), 2.39 (dd, J = 4.0, 12.0 Hz, 1H), 2.19-2.12 (m, 1H). MS: (ESI) m/z calculated for C₂₀H₁₅N₃O₃ [M + H]⁺ 346.1, found 346.2. |
| 330 | 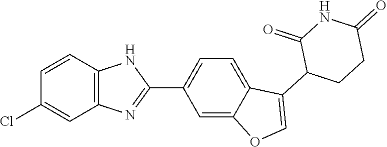 [SMILES: O=C1CCC(c2coc3cc(-c4nc5cc(Cl)ccc5[nH]4)ccc23)C(=O)N1] | ¹HNMR (400 MHz, d₆-DMSO) δ 13.18-13.01 (m, 1H), 10.95 (br s, 1H), 8.35 (d, J = 0.8 Hz, 1H), 8.10 (dd, J = 1.2, 8.4 Hz, 1H), 8.06 (s, 1H), 7.78 (d, J = 8.4 Hz, 1H), 7.71-7.51 (m, 2H), 7.23 (dd, J = 2.0, 8.4 Hz, 1H), 4.21 (dd, J = 4.8, 12.0 Hz, 1H), 2.75 (br dd, J = 5.2, 12.0 Hz, 1H), 2.62 (br d, J = 3.6 Hz, 1H), 2.38 (br dd, J = 4.0, 12.4 Hz, 1H), 2.19-2.11 (m, 1H). MS: (ESI) m/z calculated for C₂₀H₁₄ClN₃O₃ [M + H]⁺ 380.1, found 380.1. |
| 331 | 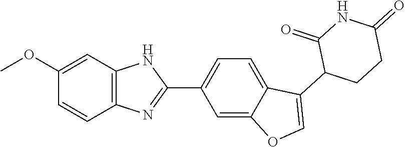 [SMILES: COc1ccc2nc(-c3ccc4c(C5CCC(=O)NC5=O)coc4c3)[nH]c2c1] | ¹HNMR (400 MHz, d₆-DMSO) δ 12.78 (br d, J = 8.0 Hz, 1H), 10.94 (br s, 1H), 8.29 (d, J = 7.2 Hz, 1H), 8.09-8.02 (m, 2H), 7.78-7.71 (m, 1H), 7.54 (d, J = 8.8 Hz, 1H), 7.42-7.00 (m, 1H), 6.88-6.80 (m, 1H), 4.20 (dd, J = 4.8, 12.4 Hz, 1H), 3.83-3.79 (m, 3H), 2.81-2.72 (m, 1H), 2.62 (br d, J = 3.6 Hz, 1H), 2.41-2.36 (m, 1H), 2.19-2.12 (m, 1H). MS: (ESI) m/z [M + H]⁺ 376.1. |
| 332 | 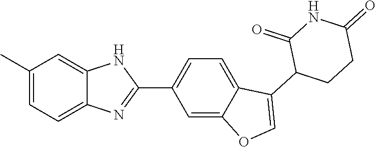 [SMILES: Cc1ccc2nc(-c3ccc4c(C5CCC(=O)NC5=O)coc4c3)[nH]c2c1] | ¹H NMR (400 MHz, d₆-DMSO) δ 12.78 (br d, J = 14.4 Hz, 1H), 10.94 (s, 1H), 8.32 (s, 1H), 8.09 (d, J = 8.0 Hz, 1H), 8.04 (s, 1H), 7.75 (d, J = 8.0 Hz, 1H), 7.53 (d, J = 8.4 Hz, 1H), 7.45-7.31 (m, 1H), 7.03 (dd, J = 8.0, 12.0 Hz, 1H), 4.20 (dd, J = 4.8, 12.4 Hz, 1H), 2.75 (br dd, J = 5.6, 12.4 Hz, 1H), 2.64-2.57 (m, 1H), 2.43 (d, J = 8.4 Hz, 3H), 2.38 (dd, J = 4.0, 12.4 Hz, 1H), 2.18-2.12 (m, 1H). MS: (ESI) m/z [M + H]⁺ 360.1. |
| 333 | 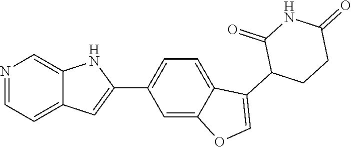 [SMILES: O=C1CCC(c2coc3cc(-c4cc5ccncc5[nH]4)ccc23)C(=O)N1] | ¹H NMR (400 MHz, DMSO-d₆) δ 12.08 (s, 1H), 10.93 (s, 1H), 8.26 (s, 1H), 8.17 (s, 1H), 8.09 (d, J = 5.2 Hz, 1H), 8.00 (s, 1H), 7.85 (d, J = 8.4 Hz, 1H), 7.72 (d, J = 8.0 Hz, 1H), 7.50 (d, J = 5.2 Hz, 1H), 7.05 (s, 1H), 4.16-4.21 (m, 1H), 2.75-2.82 (m, 1H), 2.56-2.59 (m, 1H), 2.34-2.40 (m, 1H), 2.11-2.19 (m, 1H). LCMS: calc. for C₂₀H₁₅N₃O₃: 345.1, found: [M + H]⁺ 346.0. |
| 334 | 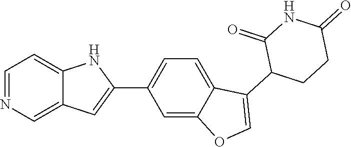 [SMILES: O=C1CCC(c2coc3cc(-c4cc5cnccc5[nH]4)ccc23)C(=O)N1] | ¹H NMR (400 MHz, DMSO-d₆) δ 12.02 (s, 1H), 10.93 (s, 1H), 8.30 (s, 1H), 8.18 (d, J = 5.6 Hz, 1H), 8.13 (s, 1H), 7.98 (s, 1H), 7.83 (d, J = 8.4 Hz, 1H), 7.70 (d, J = 8.4 Hz, 1H), 7.38 (d, J = 6.0 Hz, 1H), 7.13 (s, 1H), 4.15-4.20 (m, 1H), 2.55- 2.65 (m, 2H), 2.32-2.40 (m, 2H). LCMS: calc. for C₂₀H₁₅N₃O₃: 345.1 found: [M + H]⁺ 346.0. |
| 335 | 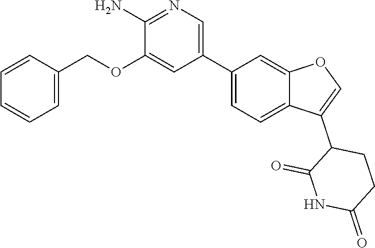 [SMILES: Nc1ncc(-c2ccc3c(C4CCC(=O)NC4=O)coc3c2)cc1OCc1ccccc1] | ¹H NMR (400 MHz, DMSO-d₆) δ 10.91 (s, 1H), 7.93 (d, J = 2.0 Hz, 1H), 7.89 (s, 1H), 7.80 (d, J = 1.2 Hz, 1H), 7.52-7.63 (m, 3H), 7.45-7.51 (m, 2H), 7.41 (t, J = 7.6 Hz, 2H), 7.29-7.37 (m, 1H), 5.86 (s, 2H), 5.28 (s, 2H), 4.15 (dd, J = 12.0, 4.4 Hz, 1H), 2.70-2.87 (m, 1H), 2.29-2.39 (m, 2H), 2.09-2.19 (m, 1H). LCMS: calc. for C₂₅H₂₁N₃O₄: 427.2, found: [M + H]⁺ 428.1. |
| 336 | 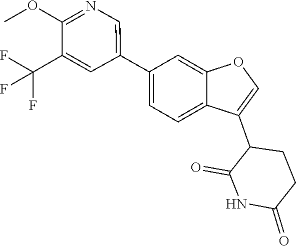 [SMILES: COc1ncc(-c2ccc3c(C4CCC(=O)NC4=O)coc3c2)cc1C(F)(F)F] | ¹H NMR (400 MHz, DMSO-d₆) δ 10.93 (s, 1H), 8.84 (d, J = 2.0 Hz, 1H), 8.39 (d, J = 2.4 Hz, 1H), 8.03 (s, 1H), 7.98 (s, 1H), 7.67-7.72 (m, 1H), 7.59-7.67 (m, 1H), 4.18 (dd, J = 12.0, 4.8 Hz, 1H), 4.04 (s, 3H), 2.70-2.83 (m, 1H), 2.55-2.63 (m, 1H), 2.31-2.39 (m, 1H), 2.09-2.19 (m, 1H). LCMS: calc. for C₂₀H₁₅F₃N₂O₄: 404.1, found: [M + H]⁺ 405.1. |
| 337 | 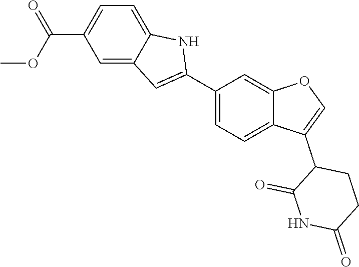 [SMILES: COC(=O)c1ccc2[nH]c(-c3ccc4c(C5CCC(=O)NC5=O)coc4c3)cc2c1] | 1H NMR (400 MHz, DMSO-d₆) δ 11.97 (s, 1H), 10.94 (s, 1H), 8.13 (s, 1H), 8.05 (s, 1H), 7.99 (s, 1H), 7.83 (dd, J = 8.4, 1.2 Hz, 1H), 7.71 (d, J = 8.0 Hz, 1H), 7.63 (s, 2H), 7.10 (d, J = 0.8 Hz, 1H), 4.19 (dd, J = 12.4, 4.8 Hz, 1H), 3.87 (s, 3H), 2.72-2.82 (m, 1H), 2.57-2.64 (m, 1H), 2.35-2.40 (m, 1H), 2.12-2.20 (m, 1H). LCMS: calc. for C₂₃H₁₈N₂O₅: 402.1, found: [M + H]⁺ 403.1. |
| 338 | 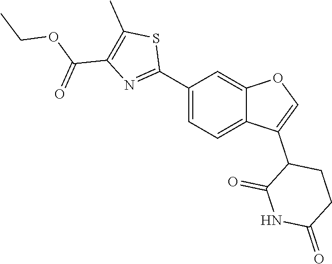 [SMILES: CCOC(=O)c1nc(-c2ccc3c(C4CCC(=O)NC4=O)coc3c2)sc1C] | ¹H NMR (400 MHz, DMSO-d₆) δ 10.94 (s, 1H), 8.09 (s, 1H), 8.06 (s, 1H), 7.79-7.83 (m, 1H), 7.71-7.74 (m, 1H), 4.30-4.34 (m, 2H), 4.20 (dd, J = 12.4, 4.4 Hz, 1H), 2.76 (s, 3H), 2.58-2.64 (m, 1H), 2.52 (d, J = 1.6 Hz, 1H), 2.29-2.36 (m, 1H), 2.09-2.20 (m, 1H), 1.35 (t, J = 7.2 Hz, 3H). LCMS: calc. for C₂₀H₁₈N₂O₅S: 398.1, found: [M + H]⁺ 399.1. |
| 339 | 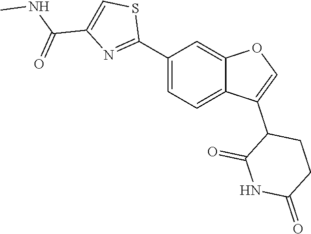 [SMILES: CNC(=O)c1csc(-c2ccc3c(C4CCC(=O)NC4=O)coc3c2)n1] | ¹H NMR (400 MHz, DMSO-d₆) δ 10.95 (s, 1H), 8.58 (s, 1H), 8.34 (s, 1H), 8.26 (s, 1H), 8.07 (s, 1H), 7.93 (dd, J = 8.4, 1.2 Hz, 1H), 7.75 (d, J = 8.4 Hz, 1H), 4.21 (dd, J = 12.4, 4.4 Hz, 1H), 2.84 (s, 3H), 2.72-2.79 (m, 1H), 2.28-2.63 (m, 1H), 2.30-2.37 (m, 1H), 2.09-2.21 (m, 1H). LCMS: calc. for C₁₈H₁₅N₃O₄S: 369.1, found: [M + H]⁺ 370.2. |
| 340 | 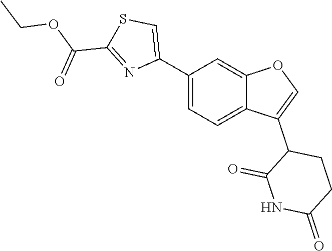 [SMILES: CCOC(=O)c1nc(-c2ccc3c(C4CCC(=O)NC4=O)coc3c2)cs1] | ¹H NMR (400 MHz, DMSO-d₆) δ 10.93 (s, 1H), 8.60 (s, 1H), 8.20 (s, 1H), 7.99 (s, 1H), 7.94 (dd, J = 8.4, 1.2 Hz, 1H), 7.70 (d, J = 8.4 Hz, 1H), 4.43 (q, J = 7.2 Hz, 2H), 4.18 (dd, J = 12.0, 4.8 Hz, 1H), 2.69-2.85 (m, 1H), 2.55-2.65 (m, 1H), 2.28-2.42 (m, 1H), 2.07-2.22 (m, 1H), 1.37 (t, J = 7.2 Hz, 3H). LCMS: calc. for C₁₈H₁₄N₂O₅S: 384.1, found: [M + H]⁺ 385.1 |
| 341 | 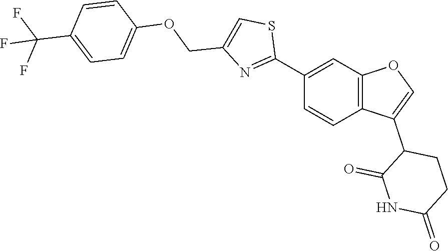 [SMILES: O=C1CCC(c2coc3cc(-c4nc(COc5ccc(C(F)(F)F)cc5)cs4)ccc23)C(=O)N1] | ¹H NMR (400 MHz, DMSO-d₆) δ: 10.95 (s, 1H), 8.15 (s, 1H), 8.06 (s, 1H), 7.82-7.88 (m, 2H), 7.66-7.75 (m, 3H), 7.28 (d, J = 8.4 Hz 2H), 5.34 (s, 2H), 4.20 (dd, J = 12.0, 4.4 Hz, 1H), 2.72-2.81 (m, 1H), 2.55-2.63 (m, 2H), 2.11-2.20 (m, 1H). LCMS: calc. for C₂₄H₁₇F₃N₂O₄S: 486.1, found: [M + H]⁺ 487.1. |
| 342 | 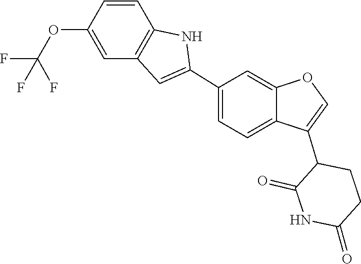 [SMILES: O=C1CCC(c2coc3cc(-c4cc5cc(OC(F)(F)F)ccc5[nH]4)ccc23)C(=O)N1] | ¹H NMR (400 MHz, DMSO-d₆) δ 11.87 (brs, 1H), 10.94 (brs, 1H), 8.09 (s, 1H), 7.97 (s, 1H), 7.79 (d, J = 7.6 Hz, 1H), 7.68 (d, J = 8.0 Hz, 1H), 7.44-7.53 (m, 2H), 7.02-7.09 (m, 2H), 4.14-4.21 (m, 1H), 2.72-2.82 (m, 1H), 2.56-2.65 (m, 2H), 2.10-2.19 (m, 1H). LCMS: calc. for C₂₂H₁₅F₃N₂O₄: 428.1, found: [M + H]⁺ 429.2. |
| 343 | 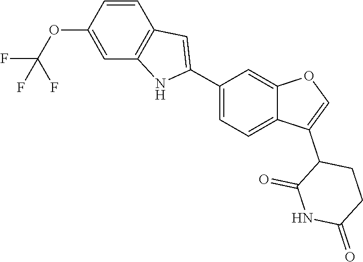 [SMILES: O=C1CCC(c2coc3cc(-c4cc5ccc(OC(F)(F)F)cc5[nH]4)ccc23)C(=O)N1] | ¹H NMR (400 MHz, DMSO-d₆) δ: 11.87 (s, 1H), 10.93 (s, 1H), 8.08 (s, 1H), 7.97 (s, 1H), 7.75- 7.80 (m, 1H), 7.66-7.70 (m, 1H), 7.62 (d, J = 8.4 Hz 1H), 7.33 (s, 1H), 7.05 (s, 1H), 6.99 (d, J = 9.2 Hz 1H), 4.17 (dd, J = 12.0, 4.4 Hz, 1H), 2.72- 2.81 (m, 1H), 2.55-2.63 (m, 2H), 2.11-2.20 (m, 1H). LCMS: calc. for C₂₂H₁₅F₃N₂O₄: 428.1, found: [M + H]⁺ 429.0 |
| 344 | 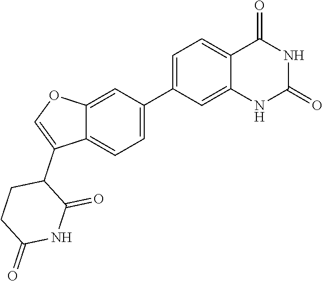 [SMILES: O=C1CCC(c2coc3cc(-c4ccc5c(=O)[nH]c(=O)[nH]c5c4)ccc23)C(=O)N1] | ¹H NMR (400 MHz, DMSO-d₆) δ 11.30 (s, 1H), 11.19 (s, 1H), 10.93 (s, 1H), 8.01 (s, 1H), 7.96 (d, J = 8.4 Hz, 1H), 7.90 (d, J = 0.8 Hz, 1H), 7.73 (d, J = 8.4 Hz, 1H), 7.52-7.59 (m, 2H), 7.44 (d, J = 1.2 Hz, 1H), 4.19 (dd, J = 12.0, 4.8 Hz, 1H), 2.72-2.83 (m, 1H), 2.58-2.65 (m, 1H), 2.34-2.42 (m, 1H), 2.12-2.18 (m, 1H). LCMS: calc. for C₂₁H₁₅N₃O₅: 389.4, found: [M + H]⁺ 390.1. |
| 345 | 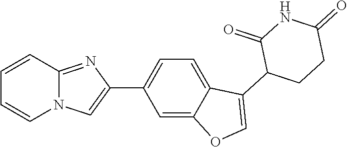 [SMILES: O=C1CCC(c2coc3cc(-c4cn5ccccc5n4)ccc23)C(=O)N1] | ¹H NMR (400 MHz, DMSO-d₆) δ 10.93 (s, 1H), 8.52 (d, J = 6.4 Hz, 1H), 8.47 (s, 1H), 8.31 (s, 0.6H, FA), 8.14 (d, J = 0.8 Hz, 1H), 7.94 (s, 1H), 7.89 (dd, J = 8.4, 1.2 Hz, 1H), 7.64 (d, J = 8.0 Hz, 1H), 7.59 (d, J = 8.8 Hz, 1H), 7.21-7.26 (m, 1H), 6.91-6.93 (m, 1H), 4.12-4.20 (m, 1H), 2.70- 2.81 (m, 1H), 2.60-2.64 (m, 1H), 2.30-2.36 (m, 1H), 2.09-2.19 (m, 1H). LCMS: calc. for C₂₀H₁₅N₃O₃: 345.1, found: [M + H]⁺ 346.0. |
| 346 | 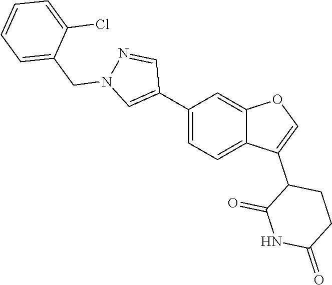 [SMILES: O=C1CCC(c2coc3cc(-c4cnn(Cc5ccccc5Cl)c4)ccc23)C(=O)N1] | ¹H NMR (400 MHz, DMSO-d₆) δ 10.96-10.86 (m, 1H), 8.35 (s, 1H), 8.03 (s, 1H), 7.86 (s, 1H), 7.82 (s, 1H), 7.58-7.54 (m, 1H), 7.53-7.47 (m, 2H), 7.40-7.31 (m, 2H), 7.08 (dd, J = 2.0, 7.2 Hz, 1H), 5.46 (s, 2H), 4.13 (dd, J = 4.0, 12.0 Hz, 1H), 2.79-2.69 (m, 1H), 2.61-2.55 (m, 1H), 2.38-2.28 (m, 1H), 2.16-2.09 (m, 1H). MS: (ESI) m/z [M + H]⁺ 420.2. |
| 347 | 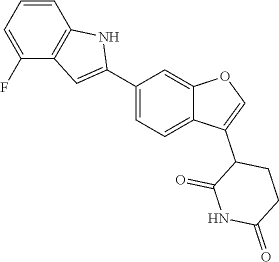 [SMILES: O=C1CCC(c2coc3cc(-c4cc5c(F)cccc5[nH]4)ccc23)C(=O)N1] | ¹H NMR (400 MHz, DMSO-d₆) δ 11.88 (s, 1 H), 10.93 (s, 1 H), 8.12 (s, 1H), 7.97 (s, 1 H), 7.82 (d, J = 8.4 Hz, 1H), 7.67 (d, J = 8.0 Hz, 1H), 7.25 (d, J = 8.0 Hz, 1H), 7.08-7.06 (m, 2H), 6.81-6.76 (m, 1H), 4.20-4.15 (m, 1H), 2.77-2.75 (m, 1H), 2.73-2.72 (m, 1H), 2.39-2.34 (m, 1H), 2.17- 2.13 (m, 1H). MS: (ESI) m/z [M + H]⁺ 363.0. |
| 348 | 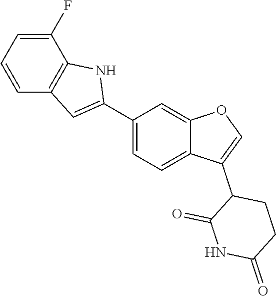 [SMILES: O=C1CCC(c2coc3cc(-c4cc5cccc(F)c5[nH]4)ccc23)C(=O)N1] | ¹H NMR (400 MHz, DMSO-d₆) δ 11.88 (s, 1H), 10.92 (br s, 1H), 8.19 (d, J = 1.6 Hz, 1H), 7.96 (s, 1H), 7.86 (dd, J = 8.0, 1.6 Hz, 1H), 7.66 (d, J = 8.2 Hz, 1H), 7.36 (d, J = 7.6 Hz, 1H), 7.10-7.04 (m, 1H), 7.02-6.96 (m, 1H), 6.96-6.89 (m, 1H), 4.17 (dd, J = 12.0, 4.0 Hz, 1H), 2.83-2.71 (m, 1H), 2.60 (dt, J = 16.0, 3.69 Hz, 1H), 2.43-2.30 (m, 1H), 2.20-2.10 (m, 1H). MS: (ESI) m/z [M + H]⁺ 363.0. |
| 349 | 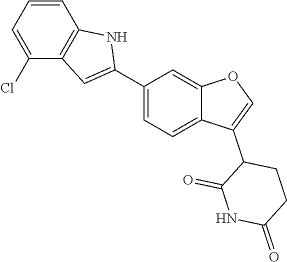 [SMILES: O=C1CCC(c2coc3cc(-c4cc5c(Cl)cccc5[nH]4)ccc23)C(=O)N1] | ¹H NMR (400 MHz, DMSO-d₆) δ 11.93 (d, J = 1.6 Hz, 1H), 10.93 (s, 1H), 8.14 (d, J = 0.8 Hz, 1H), 7.97 (s, 1H), 7.84 (dd, J = 1.2, 8.0 Hz, 1H), 7.68 (d, J = 8.0 Hz, 1H), 7.42-7.34 (m, 1H), 7.14-7.05 (m, 2H), 7.02 (d, J = 1.6 Hz, 1H), 4.18 (dd, J = 4.8, 12.0 Hz, 1H), 2.77 (s, 1H), 2.60 (br d, J = 17.2 Hz, 1H), 2.37 (br dd, J = 4.0, 12.4 Hz, 1H), 2.15 (m, 1H). MS: (ESI) m/z [M + H]⁺ 379.3. |
| 350 | 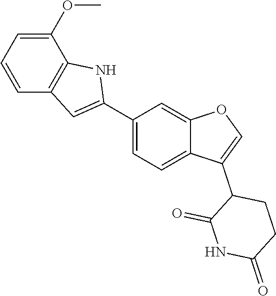 [SMILES: COc1cccc2cc(-c3ccc4c(C5CCC(=O)NC5=O)coc4c3)[nH]c12] | ¹H NMR (400 MHz, DMSO-d₆) δ 11.50 (br s, 1H), 10.93 (br s, 1H), 8.21 (s, 1H), 7.93 (s, 1H), 7.89-7.81 (m, 1H), 7.61 (d, J = 8.3 Hz, 1H), 7.12 (d, J = 7.9 Hz, 1H), 6.98-6.88 (m, 2H), 6.68 (d, J = 7.6 Hz, 1H), 4.16 (br dd, J = 4.8, 12.0 Hz, 1H), 3.96 (s, 3H), 2.82-2.70 (m, 1H), 2.64-2.55 (m, 1H), 2.42-2.29 (m, 1H), 2.19- 2.09 (m, 1H). MS: (ESI) m/z [M + H]⁺ 375.3. |
| 351 | 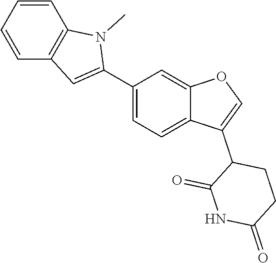 | ¹H NMR (400 MHz, DMSO-d₆) δ 10.95 (br s, 1H), 8.03-7.98 (m, 1H), 7.84-7.80 (m, 1H), 7.73-7.69 (m, 1H), 7.60-7.55 (m, 1H), 7.52- 7.46 (m, 2H), 7.22-7.16 (m, 1H), 7.11-7.04 (m, 1H), 6.61 (s, 1H), 4.24-4.15 (m, 1H), 3.79 (s, 3H), 2.83-2.72 (m, 1H), 2.64-2.57 (m, 1H), 2.37 (br s, 1H), 2.19-2.10 (m, 1H). MS: (ESI) m/z [M + H]⁺ 379.3. |
| 352 | 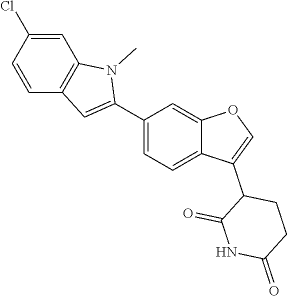 [SMILES: Cn1c(-c2ccc3c(C4CCC(=O)NC4=O)coc3c2)cc2ccc(Cl)cc21] | ¹H NMR (400 MHz, DMSO-d₆) δ 10.94 (s, 1H), 8.01 (s, 1H), 7.82 (d, J = 0.8 Hz, 1H), 7.72 (d, J = 8.0 Hz, 1H), 7.65 (s, 1H), 7.58 (d, J = 8.4 Hz, 1H), 7.48 (dd, J = 8.0,1.6 Hz, 1H), 7.09 (dd, J = 8.4, 2.0Hz, 1H), 6.64 (s, 1H), 4.20 (dd, J = 12.4,4.8 Hz, 1H), 3.78 (s, 3H), 2.85-2.72 (m, 1H), 2.68-2.56 (m, 1H), 2.44-2.31 (m, 1H), 2.23-2.11 (m, 1H). MS: (ESI) m/z [M + H]⁺ 393.3. |
| 353 | 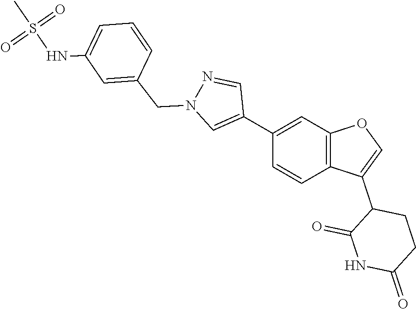 [SMILES: CS(=O)(=O)Nc1cccc(Cn2cc(-c3ccc4c(C5CCC(=O)NC5=O)coc4c3)cn2)c1] | ¹H NMR (400 MHz, DMSO-d₆) δ 10.90 (s, 1H), 9.80 (s, 1H), 8.34 (s, 1H), 8.00 (s, 1H), 7.92- 7.69 (m, 2H), 7.59-7.52 (m, 1H), 7.51-7.45 (m, 1H), 7.35-7.28 (m, 1H), 7.18-7.11 (m, 2H), 7.00 (d, J = 7.6 Hz, 1H), 5.33 (s, 2H), 4.13 (dd, J = 4.8, 11.6 Hz, 1H), 3.01-2.94 (m, 3H), 2.83- 2.68 (m, 1H), 2.64-2.53 (m, 1H), 2.32 (dt, J = 8.4, 12.5 Hz, 1H), 2.19-2.02 (m, 1H). MS: (ESI) m/z [M + H]⁺ 479.1. |
| 354 | 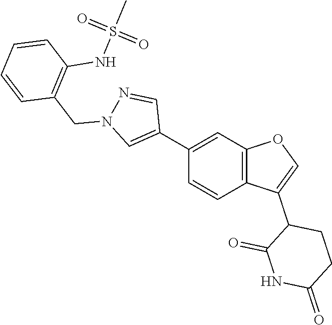 [SMILES: CS(=O)(=O)Nc1ccccc1Cn1cc(-c2ccc3c(C4CCC(=O)NC4=O)coc3c2)cn1] | ¹H NMR (400 MHz, DMSO-d₆) δ 10.90 (s, 1H), 9.51 (s, 1H), 8.34 (s, 1H), 8.04 (s, 1H), 7.86 (s, 1H), 7.80 (d, J = 0.6 Hz, 1H), 7.58-7.52 (m, 1H), 7.51-7.45 (m, 1H), 7.43-7.38 (m, 1H), 7.35 (dt, J = 1.4, 7.6 Hz, 1H), 7.24 (dt, J = 1.3, 7.5 Hz, 1H), 7.04 (dd, J = 4.8, 11.6Hz, 1H), 5.49 (s, 2H), 4.13 (dd, J = 4.9, 11.9 Hz, 1H), 3.03 (s, 3H), 2.81-2.68 (m, 1H), 2.58 (td, J = 3.8, 17.3 Hz, 1H), 2.32 (br dd, J = 4.1, 12.6 Hz, 1H), 2.18- 2.05 (m, 1H). MS: (ESI) m/z [M + H]⁺ 479.3. |
| 355 | 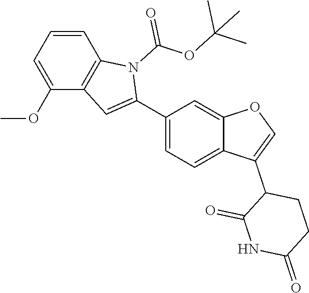 [SMILES: COc1cccc2c1cc(-c1ccc3c(C4CCC(=O)NC4=O)coc3c1)n2C(=O)OC(C)(C)C] | ¹H NMR (400 MHz, DMSO-d₆) δ 10.93 (s, 1H), 7.96 (s, 1H), 7.68 (t, J = 4.0 Hz, 2H), 7.62 (d, J = 8.0 Hz, 1H), 7.24-7.34 (m, 2H), 6.82 (d, J = 8.0 Hz, 1H), 6.73 (s, 1H), 4.18 (dd, J = 12.0, 4.8 Hz, 1H), 3.90 (s, 3H), 2.72-2.82 (m, 1H), 2.55-2.63 (m, 1H), 2.55-2.63 (m, 1H), 2.28-2.42 (m, 1H), 1.24 (s, 9H). LCMS: calc. for C₂₇H₂₆N₂O₆: 474.2, found: [M + H]⁺ 475.1. |
| 356 | 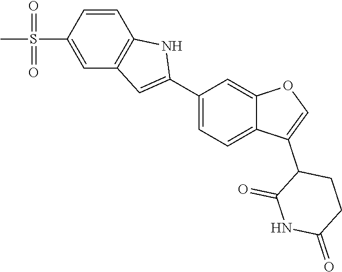 [SMILES: CS(=O)(=O)c1ccc2[nH]c(-c3ccc4c(C5CCC(=O)NC5=O)coc4c3)cc2c1] | ¹H NMR (400 MHz, DMSO-d₆) δ 10.80 (s, 1H), 8.30 (s, 1H), 7.99-7.96 (m, 1H), 7.85 (s, 1H), 7.79 (s, 1H), 7.56-7.52 (m, 1H), 7.49-7.46 (m, 1H), 7.23-7.17 (m, 4H), 5.29 (s, 2H), 4.14-4.10 (m, 1H), 2.61-2.57 (m, 2H), 2.57-2.54 (m, 2H), 2.36-2.29 (m, 1H), 2.16-2.08 (m, 1H), 2.17- 2.08 (m, 1H), 1.15 (t, J = 7.6 Hz, 3H). MS: (ESI) m/z [M + H]⁺ 423.1. |
| 357 | 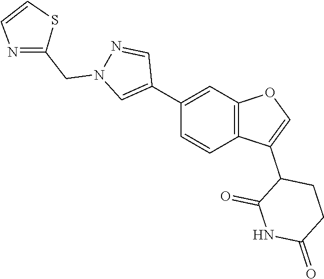 [SMILES: O=C1CCC(c2coc3cc(-c4cnn(Cc5nccs5)c4)ccc23)C(=O)N1] | ¹H NMR (400 MHz, DMSO-d₆) δ 10.92-10.86 (m, 1H), 8.43-8.39 (m, 1H), 8.08-8.06 (m, 1H), 7.88-7.85 (m, 1H), 7.83-7.79 (m, 2H), 7.72- 7.70 (m, 1H), 7.58-7.54 (m, 1H), 7.52-7.48 (m, 1H), 5.72 (s, 2H), 4.13 (dd, J = 4.8, 12.0 Hz, 1H), 2.76-2.70 (m, 1H), 2.60 (d, J = 4.0 Hz, 1H), 2.32 (s, 1H), 2.15 (d, J = 4.0 Hz, 1H). MS: (ESI) m/z [M + H]⁺ 393.1. |
| 358 | 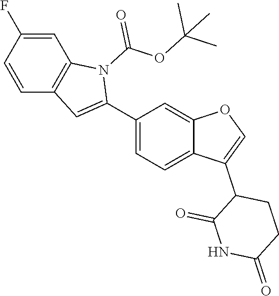 [SMILES: CC(C)(C)OC(=O)n1c(-c2ccc3c(C4CCC(=O)NC4=O)coc3c2)cc2ccc(F)cc21] | ¹H NMR (400 MHz, DMSO-d₆) δ 10.93 (s, 1H), 7.97 (s, 1H), 7.83 (dd, J = 10.8, 1.6 Hz, 1H), 7.69 (s, 1H), 7.62-7.67 (m, 2H), 7.32 (d, J = 8.0 Hz, 1H), 7.12-7.19 (m, 1H), 6.78 (s, 1H), 4.19 (dd, J = 12.0, 4.4 Hz, 1H), 2.72-2.82 (m, 1H), 2.55-2.63 (m, 1H), 2.31-2.41 (m, 1H), 2.10-2.19 (m, 1H), 1.23 (s, 9H). LCMS: calc. for C₂₆H₂₃ClN₂O₅: 462.2, found: [M − Boc + H]⁺ 363.1. |
| 359 | 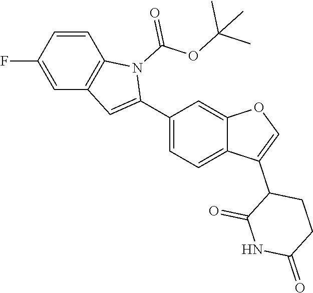 [SMILES: CC(C)(C)OC(=O)n1c(-c2ccc3c(C4CCC(=O)NC4=O)coc3c2)cc2cc(F)ccc21] | ¹H NMR (400 MHz, DMSO-d₆) δ 10.93 (s, 1H), 8.08 (dd, J = 9.2, 4.4 Hz, 1H), 7.98 (s, 1H), 7.70 (s, 1H), 7.64 (d, J = 8.0 Hz, 1H), 7.43 (dd, J = 9.2, 2.4 Hz, 1H), 7.33 (d, J = 8.0 Hz, 1H), 7.18 (td, J = 9.2, 2.4 Hz, 1H), 6.76 (s, 1H), 4.19 (dd, J = 12.0, 4.8 Hz, 1H), 2.72-2.82 (m, 1H), 2.55-2.64 (m, 1H), 2.30-2.40 (m, 1H), 2.10-2.19 (m, 1H), 1.23 (s, 9H). LCMS: calc. for C₂₆H₂₃FN₂O₅: 462.1, found: [M − Boc + H]⁺ 363.1. |
| 360 | 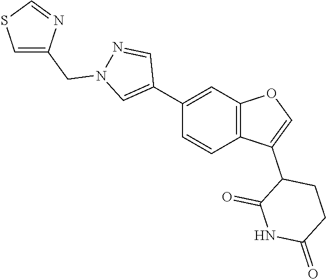 [SMILES: O=C1CCC(c2coc3cc(-c4cnn(Cc5cscn5)c4)ccc23)C(=O)N1] | ¹HNMR (400 MHz, DMSO-d₆) δ 10.89 (s, 1H), 9.09 (d, J = 1.6Hz, 1H), 8.31 (s, 1H), 7.98 (s, 1H), 7.85 (s, 1H), 7.80 (s, 1H), 7.57-7.61 (m, 1H), 7.53-7.57 (m, 1H), 7.46-7.51 (m, 1H), 5.48 (s, 2H), 4.13 (dd, J = 12.0, 4.8Hz, 1H), 2.74 (m, 1H), 2.57 (m, 1H), 2.32 (m, 1H), 2.09-2.17 (m, 1H). MS: (ESI) m/z [M + H]⁺ 392.9. |
| 361 | 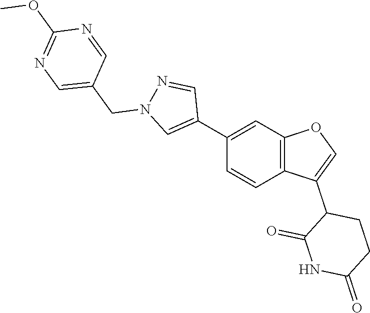 [SMILES: COc1ncc(Cn2cc(-c3ccc4c(C5CCC(=O)NC5=O)coc4c3)cn2)cn1] | ¹H NMR (400 MHz, DMSO-d₆) δ 10.89 (br s, 1H), 8.65 (s, 2H), 8.35 (s, 1H), 7.99 (s, 1H), 7.86 (s, 1H), 7.79 (s, 1H), 7.58-7.53 (m, 1H), 7.47 (dd, J = 1.2, 8.0 Hz, 1H), 5.34 (s, 2H), 4.13 (dd, J = 4.8, 12.0 Hz, 1H), 3.91 (s, 3H), 2.79-2.68 (m, 1H), 2.58 (td, J = 4.0, 17.2 Hz, 1H), 2.38-2.27 (m, 1H), 2.16-2.08 (m, 1H). MS: (ESI) m/z [M + H]⁺ 418.0. |
| 362 | 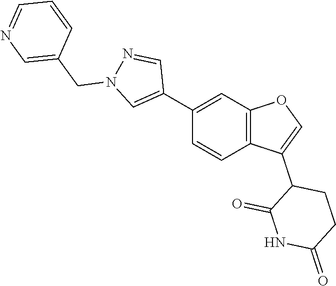 [SMILES: O=C1CCC(c2coc3cc(-c4cnn(Cc5cccnc5)c4)ccc23)C(=O)N1] | ¹H NMR (400 MHz, DMSO-d₆) δ 10.91 (br s, 1H), 8.59 (d, J = 1.6 Hz, 1H), 8.54 (dd, J = 1.2, 4.8Hz, 1H), 8.40 (s, 1H), 8.03 (s, 1H), 7.88 (s, 1H), 7.82 (s, 1H), 7.75-7.69 (m, 1H), 7.60-7.54 (m, 1H), 7.54-7.47 (m, 1H), 7.41 (dd, J = 4.8, 12.0 Hz, 1H), 5.43 (s, 2H), 4.15 (dd, J = 4.8, 12.0 Hz, 1H), 2.83-2.68 (m, 1H), 2.60 (td, J = 3.9, 17.2 Hz, 1H), 2.34 (dq, J = 4.4, 12.4 Hz, 1H), 2.24-2.05 (m, 1H). MS: (ESI) m/z [M + H]⁺ 387.1. |
| 363 | 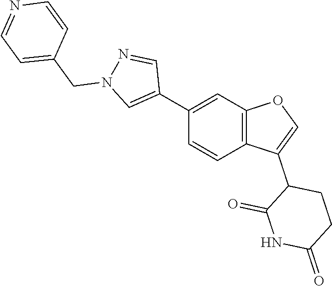 [SMILES: O=C1CCC(c2coc3cc(-c4cnn(Cc5ccncc5)c4)ccc23)C(=O)N1] | ¹H NMR (400 MHz, DMSO-d₆) δ 10.91 (br s, 1H), 8.54 (d, J = 5.6 Hz, 2H), 8.39 (s, 1H), 8.06 (s, 1H), 7.84 (d, J = 16.0 Hz, 2H), 7.63-7.53 (m, 1H), 7.53-7.47 (m, 1H), 7.19 (d, J = 5.6 Hz, 2H), 5.43 (s, 2H), 4.13 (dd, J = 4.8, 12.0 Hz, 1H), 2.80-2.70 (m, 1H), 2.58 (td, J = 3.6, 16.0 Hz, 2H), 2.33 (br dd, J = 4.0, 12.4 Hz, 1H), 2.17- 2.08 (m, 1H). MS: (ESI) m/z [M + H]⁺ 387.2. |
| 364 | 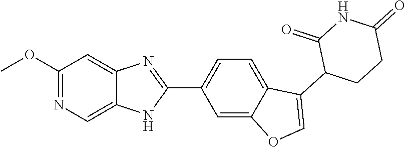 [SMILES: COc1cc2nc(-c3ccc4c(C5CCC(=O)NC5=O)coc4c3)[nH]c2cn1] | ¹H NMR (400 MHz, DMSO-d₆) δ 13.06 (br s, 1H), 10.96 (s, 1H), 8.55 (s, 1H), 8.35 (s, 1H), 8.06-8.14 (m, 2H), 7.78 (d, J = 8.4 Hz, 1H), 6.86 (s, 1H), 4.21 (dd, J = 12.0, 4.8 Hz, 1H), 3.89 (s, 3H), 2.72-2.86 (m, 1H), 2.54-2.60 (m, 1H), 2.30- 2.46 (m, 1H), 2.09-2.19 (m, 1H). LCMS: calc. for C₂₀H₁₆N₄O₄: 376.1, found: [M + H]⁺ 377.1. |
| 365 | 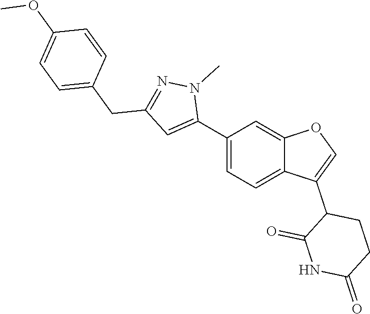 [SMILES: COc1ccc(Cc2cc(-c3ccc4c(C5CCC(=O)NC5=O)coc4c3)n(C)n2)cc1] | ¹H NMR (400 MHz, DMSO-d₆) δ 10.91 (s, 1H), 7.98 (s, 1H), 7.72 (s, 1H), 7.66 (d, J = 8.0 Hz, 1H), 7.35 (dd, J = 8.0, 1.2 Hz, 1H), 7.20 (d, J = 8.8 Hz, 2H), 6.85 (d, J = 8.8 Hz, 2H), 6.18 (s, 1H), 4.17 (dd, J = 12.0, 5.2 Hz, 1H), 3.82 (s, 5H), 3.71 (s, 3H), 2.70-2.81 (m, 1H), 2.58-2.63 (m, 1H), 2.35-2.40 (m, 1H), 2.09-2.17 (m, 1H). LCMS: calc. for C₂₅H₂₃N₃O₄: 429.2, found: [M + H]⁺ 430.2. |
| 366 | 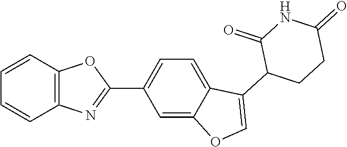 [SMILES: O=C1CCC(c2coc3cc(-c4nc5ccccc5o4)ccc23)C(=O)N1] | ¹H NMR (400 MHz, DMSO-d₆) δ 10.94 (s, 1H), 8.37 (d, J = 0.8 Hz, 1H), 8.09-8.16 (m, 2H), 7.77- 7.87 (m, 3H), 7.39-7.49 (m, 2H), 4.24 (dd, J = 12.4, 4.8 Hz, 1H), 2.73-2.82 (m, 1H), 2.58-2.65 (m, 1H), 2.35-2.42 (m, 1H), 2.12-2.21 (m, 1H). LCMS: calc. for C₂₀H₁₄N₂O₄: 346.3, found: [M + H]⁺ 347.1. |
| 367 | 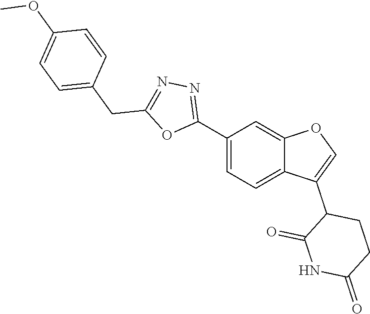 [SMILES: COc1ccc(Cc2nnc(-c3ccc4c(C5CCC(=O)NC5=O)coc4c3)o2)cc1] | ¹H NMR (400 MHz, DMSO-d₆) δ 10.95 (brs, 1H), 8.14 (s, 1H), 8.12 (s, 1H), 7.84-7.88 (m, 1H), 7.80 (d, J = 8.0 Hz, 1H), 7.33 (d, J = 8.8 Hz, 2H), 6.93 (d, J = 8.8 Hz, 2H), 4.29 (s, 2H), 4.18-4.25 (m, 1H), 3.74 (s, 3H), 2.71-2.81 (m, 1H), 2.57-2.63 (m, 1H), 2.32-2.39 (m, 1H), 2.09- 2.17 (m, 1H). LCMS: calc. for C₂₃H₁₉N₃O₅: 417.1, found: [M + H]⁺ 418.1. |
| 368 | 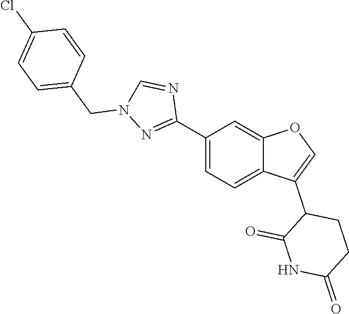 [SMILES: O=C1CCC(c2coc3cc(-c4ncn(Cc5ccc(Cl)cc5)n4)ccc23)C(=O)N1] | ¹H NMR (400 MHz, DMSO-d₆) δ: 10.92 (s, 1H), 8.74 (s, 1H), 8.08 (s, 1H), 7.97 (s, 1H), 7.87-7.91 (m, 1H), 7.64-7.67 (m, 1H), 7.36-7.47 (m, 4H), 5.48 (s, 2H), 4.17 (dd, J = 12.0, 4.4 Hz, 1H), 2.70-2.81 (m, 1H), 2.55-2.63 (m, 2H), 2.11-2.20 (m, 1H). LCMS: calc. for C₂₂H₁₇ClN₄O₃: 420.1, found: [M + H]⁺ 421.1. |
| 369 | 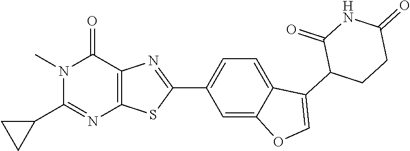 [SMILES: Cn1c(C2CC2)nc2sc(-c3ccc4c(C5CCC(=O)NC5=O)coc4c3)nc2c1=O] | ¹H NMR (400 MHz, DMSO-d₆) δ 10.96 (brs, 1H), 8.20 (s, 1H), 8.09 (s, 1H), 7.89-7.96 (m, 1H), 7.76 (d, J = 7.6 Hz, 1H), 4.15-4.25 (m, 1H), 3.73 (s, 3H), 2.56-2.74 (m, 3H), 2.30-2.40 (m, 1H), 2.10-2.15 (m, 1H), 1.08-1.17 (m, 4H). LCMS: calc. for C₂₂H₁₈N₄O₄S: 434.1, found: [M + H]⁺ 435.1. |
| 370 | 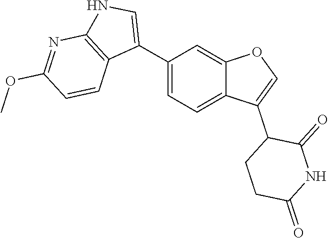 [SMILES: COc1ccc2c(-c3ccc4c(C5CCC(=O)NC5=O)coc4c3)c[nH]c2n1] | ¹H NMR (400 MHz, DMSO-d₆) δ 11.73 (brs, 1H), 10.90 (brs, 1H), 8.23 (d, J = 8.4 Hz, 1H), 7.88 (s, 1H), 7.82 (s, 1H), 7.56-7.66 (m, 2H), 6.63 (d, J = 8.4 Hz, 1H), 4.12-4.18 (m, 1H), 3.90 (s, 3H), 2.74-2.77 (m, 1H), 2.61-2.63 (m, 1H), 2.12-2.18 (m, 1H), 1.97-2.03 (m, 1H). LCMS: calc. for C₂₁H₁₇N₃O₄: 375.1, found: [M + H]⁺ 376.0. |
| 371 | 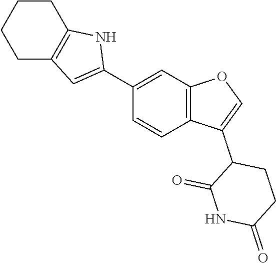 [SMILES: O=C1CCC(c2coc3cc(-c4cc5c([nH]4)CCCC5)ccc23)C(=O)N1] | ¹H NMR (400 MHz, DMSO-d₆) δ 10.89 (s, 1H), 10.75 (d, J = 1.6 Hz, 1H), 7.80 (s, 1H), 7.70 (s, 1H), 7.46 (s, 2H), 6.28 (d, J = 2.4 Hz, 1H), 4.10 (dd, J = 12.0, 4.8 Hz, 1H), 2.69-2.81 (m, 1H), 2.54-2.64 (m, 3H), 2.43 (t, J = 5.6 Hz, 2H), 2.25- 2.38 (m, 1H), 2.08-2.18 (m, 1H), 1.63-1.83 (m, 4H). LCMS: calc. for C₂₁H₂₀N₂O₃: 348.1, found: [M + H]⁺ 349.2. |
| 372 | 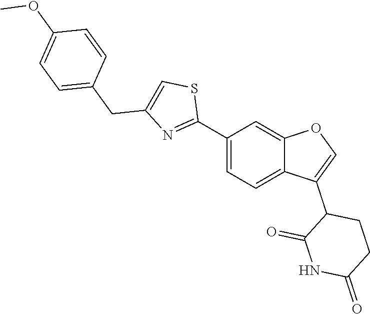 [SMILES: COc1ccc(Cc2csc(-c3ccc4c(C5CCC(=O)NC5=O)coc4c3)n2)cc1] | ¹H NMR (400 MHz, DMSO-d₆) δ 10.93 (s, 1H), 8.08 (d, J = 1.2 Hz, 1H), 8.03 (s, 1H), 7.81 (dd, J = 8.0, 1.2 Hz, 1H), 7.69 (d, J = 8.0 Hz, 1H), 7.17-7.30 (m, 3H), 6.78-6.94 (m, 2H), 4.18 (dd, J = 12.0, 5.2 Hz, 1H), 4.06 (s, 2H), 3.72 (s, 3H), 2.69-2.81 (m, 1H), 2.58-2.65 (m, 1H), 2.32-2.36 (m, 1H), 2.09-2.20 (m, 1H). LCMS: calc. for C₂₄H₂₀N₂O₄S:432.1, found: 433.1. |
| 373 | 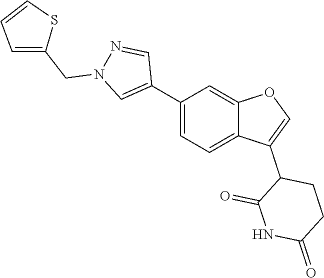 [SMILES: O=C1CCC(c2coc3cc(-c4cnn(Cc5cccs5)c4)ccc23)C(=O)N1] | ¹H NMR (400 MHz, DMSO-d₆) δ 10.80-10.99 (m, 1H) 8.41 (s, 1H) 8.32 (s, 1H) 8.00 (s, 1H) 7.86 (s, 1H) 7.80 (s, 1H) 7.52-7.58 (m, 1H) 7.45- 7.51 (m, 2H) 7.16 (d, J = 4.00 Hz, 1H) 6.99-7.04 (m, 1H) 5.54 (s, 2H) 4.10-4.17 (m, 1H) 2.62-2.81 (m, 2H) 2.27-2.33 (m, 1H) 2.10-2.18 (m, 1H). LCMS: calc. for C₂₁H₁₇N₃O₃S: 391.1, found: [M + H]⁺ 391.9. |
| 374 | 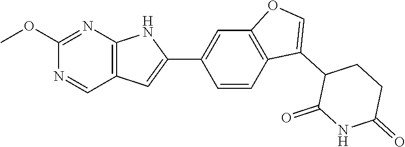 [SMILES: COc1ncc2cc(-c3ccc4c(C5CCC(=O)NC5=O)coc4c3)[nH]c2n1] | ¹H NMR (400 MHz, DMSO-d₆) δ 12.41 (s, 1H), 10.92 (s, 1H), 8.74 (s, 1H), 8.19 (s, 0.2H, FA), 8.13 (d, J = 1.2 Hz, 1H), 7.96 (s, 1H), 7.82 (dd, J = 8.4, 1.2 Hz, 1H), 7.66 (d, J = 8.4 Hz, 1H), 7.03 (s, 1H), 4.16 (dd, J = 12.4, 5.2 Hz, 1H),, 3.93 (s, 3H), 2.70-2.82 (m, 1H), 2.55-2.64 (m, 1H), 2.29- 2.42 (m, 1H), 2.09-2.19 (m, 1H). LCMS: calc. for C₂₀H₁₆N₄O₄: 376.1, found: [M + H]⁺ 377.0. |
| 375 | 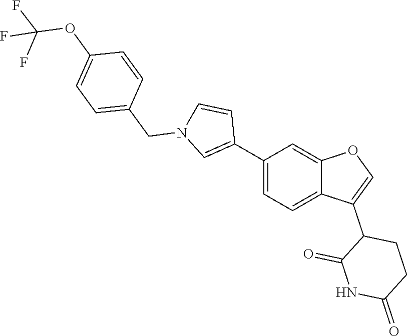 [SMILES: O=C1CCC(c2coc3cc(-c4ccn(Cc5ccc(OC(F)(F)F)cc5)c4)ccc23)C(=O)N1] | ¹H NMR (400 MHz, DMSO-d₆) δ 10.89 (s, 1H), 7.81 (s, 1H), 7.67 (s, 1H), 7.47-7.50 (m, 1H), 7.42-7.45 (m, 1H), 7.38-7.42 (m, 2H), 7.36-7.38 (m, 3H), 6.92 (t, J = 2.0 Hz, 1H), 6.52-6.56 (m, 1H), 5.17 (s, 2H), 4.08-4.14 (m, 1H), 2.70-2.77 (m, 1H), 2.58-2.62 (m, 1H), 2.31-2.35 (m, 1H), 2.11-2.16 (m, 1H). LCMS: calc. for C₂₅H₁₉F₃N₂O₄: 468.1, found: [M + H]⁺ 469.0. |
| 376 | 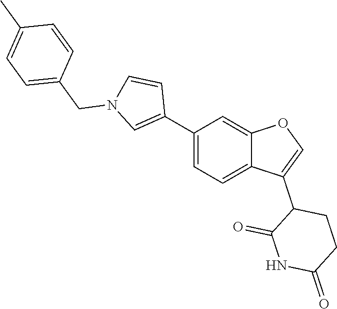 [SMILES: Cc1ccc(Cn2ccc(-c3ccc4c(C5CCC(=O)NC5=O)coc4c3)c2)cc1] | ¹H NMR (400 MHz, DMSO-d₆) δ 10.88 (s, 1H), 7.79 (s, 1H), 7.65 (s, 1H), 7.45-7.48 (m, 1H), 7.38-7.42 (m, 1H), 7.34 (t, J = 2.0 Hz, 1H), 7.16 (d, J = 2.0 Hz, 4H), 6.86 (t, J = 2.4 Hz, 1H), 6.47-6.51 (m, 1H), 5.05 (s, 2H), 4.07-4.13 (m, 1H), 2.70-2.76 (m, 1H), 2.56-2.60 (m, 1H), 2.51- 2.55 (m, 1H), 2.27 (s, 3H), 2.10-2.15 (m, 1H). LCMS: calc. for C₂₅H₂₂N₂O₃: 398.2, found: [M + H]⁺ 399.0. |
| 377 | 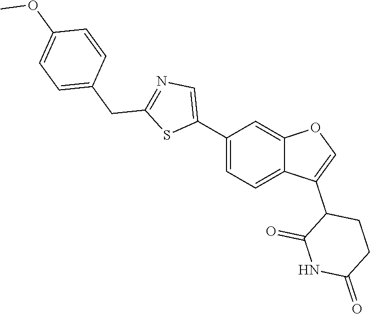 [SMILES: COc1ccc(Cc2ncc(-c3ccc4c(C5CCC(=O)NC5=O)coc4c3)s2)cc1] | ¹H NMR (400 MHz, DMSO-d₆) δ 10.91 (s, 1H), 8.13 (s, 1H), 7.94 (s, 1H), 7.85 (d, J = 1.2 Hz, 1H), 7.60 (d, J = 6.4 Hz, 1H), 7.46 (dd, J = 8.0, 1.2 Hz, 1H), 7.30 (d, J = 7.2 Hz, 2H), 6.89-6.96 (m, 2H), 4.26 (s, 2H), 4.14 (dd, J = 12.0, 4.8 Hz, 1H), 3.74 (s, 3H), 2.70-2.78 (m, 1H), 2.55-2.64 (m, 1H), 2.25-2.37 (m, 1H), 2.06-2.16 (m, 1H). LCMS: calc. for C₂₄H₂₀N₂O₄S: 432.1, found: [M + H]⁺ 433.1. |
| 378 | 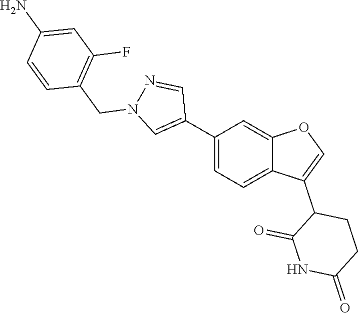 [SMILES: Nc1ccc(Cn2cc(-c3ccc4c(C5CCC(=O)NC5=O)coc4c3)cn2)c(F)c1] | ¹H NMR (400 MHz, DMSO-d₆) δ 10.89 (s, 1H), 8.28 (s, 0.15H, FA), 8.17 (s, 1H), 7.93 (s, 1H), 7.85 (s, 1H), 7.78 (s, 1H), 7.50-7.57 (m, 1H), 7.44-7.48 (m, 1H), 6.97-7.07 (m, 1H), 6.28-6.38 (m, 2H), 5.47 (s, 2H), 5.15 (s, 2H), 4.08-4.18 (m, 1H), 2.69-2.77 (m, 1H), 2.54-2.63 (m, 1H), 2.27- 2.40 (m, 1H), 2.07-2.18 (m, 1H). LCMS: calc. for C₂₃H₁₉FN₄O₃: 418.1, found: [M + H]⁺ 419.1. |
| 379 | 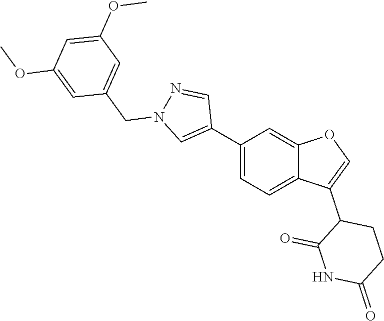 [SMILES: COc1cc(Cn2cc(-c3ccc4c(C5CCC(=O)NC5=O)coc4c3)cn2)cc(OC)c1] | ¹H NMR (400 MHz, DMSO-d₆) δ 10.89 (s, 1H), 8.33 (s, 1H), 7.99 (s, 1H), 7.83-7.89 (m, 1H), 7.80 (s, 1H), 7.44-7.59 (m, 2H), 6.44 (s, 3H), 5.26 (s, 2H), 4.13 (dd, J = 12.0, 4.8 Hz, 1H), 3.71 (s, 6H), 2.68-2.79 (m, 1H), 2.56-2.63 (m, 1H), 2.28-2.37 (m, 1H), 2.07-2.19 (m, 1H). LCMS: calc. for C₂₅H₂₃N₃O₅: 445.5, found: [M + H]⁺ 446.2. |
| 380 | 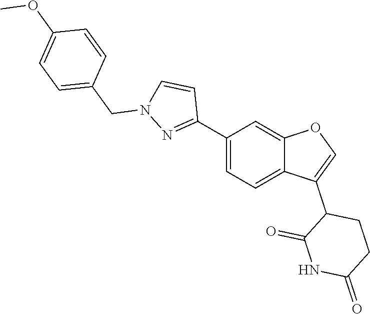 [SMILES: COc1ccc(Cn2ccc(-c3ccc4c(C5CCC(=O)NC5=O)coc4c3)n2)cc1] | ¹H NMR (400 MHz, DMSO-d₆) δ 10.90 (s, 1H), 7.93 (s, 1H), 7.90 (s, 1H), 7.82-7.85 (m, 1H), 7.69-7.74 (m, 1H), 7.55-7.61 (m, 1H), 7.24-7.30 (m, 2H), 6.88-6.95 (m, 2H), 6.78-6.82 (m, 1H), 5.29 (s, 2H), 4.11-4.18 (m, 1H), 3.73 (s, 3H), 2.69-2.81 (m, 1H), 2.54-2.63 (m, 1H), 2.25-2.40 (m, 1H), 2.08-2.19 (m, 1H). LCMS: calc. for C₂₄H₂₁N₃O₄: 415.2, found: [M + H]⁺ 416.0. |
| 381 | 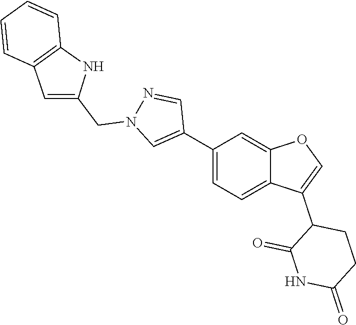 [SMILES: O=C1CCC(c2coc3cc(-c4cnn(Cc5cc6ccccc6[nH]5)c4)ccc23)C(=O)N1] | ¹H NMR (400 MHz, DMSO-d₆) δ 11.21 (s, 1H), 10.89 (s, 1H), 8.30 (s, 1H), 8.00 (s, 1H), 7.85 (s, 1H), 7.79 (s, 1H), 7.52-7.56 (m, 1H), 7.45-7.51 (m, 2H), 7.32-7.37 (m, 1H), 7.03-7.09 (m, 1H), 6.93-7.00 (m, 1H), 6.40-6.43 (m, 1H), 5.48 (s, 2H), 4.08-4.16 (m, 1H), 2.69-2.80 (m, 1H), 2.54- 2.63 (m, 1H), 2.23-2.41 (m, 1H), 2.06-2.18 (m, 1H). LCMS: calc. for C₂₅H₂₀N₄O₃: 424.2, found: [M + H]⁺ 425.2. |
| 382 | 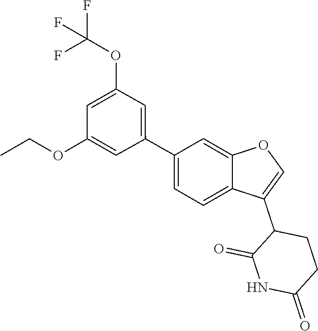 [SMILES: CCOc1cc(OC(F)(F)F)cc(-c2ccc3c(C4CCC(=O)NC4=O)coc3c2)c1] | ¹H NMR (400 MHz, DMSO-d₆) δ 10.93 (s, 1H), 7.97 (d, J = 6.4 Hz, 2H), 7.64-7.70 (m, 1H), 7.57- 7.63 (m, 1H), 7.31 (s, 1H), 7.26 (s, 1H), 6.91 (s, 1H), 4.14-4.20 (m, 3H), 2.72-2.80 (m, 1H), 2.57- 2.62 (m, 1H), 2.36 (dd, J = 12.8, 4.0 Hz, 1H), 2.10-2.19 (m, 1H), 1.36 (t, J = 6.8 Hz, 3H). LCMS: calc. for C₂₂H₁₈F₃NO₅: 433.1, found: [M + H]⁺ 434.2. |
| 383 | 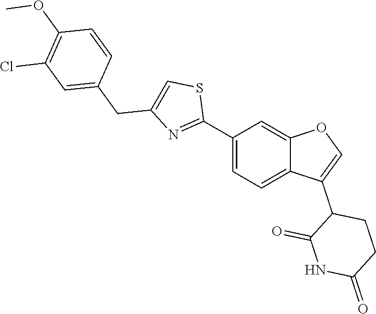 [SMILES: COc1ccc(Cc2csc(-c3ccc4c(C5CCC(=O)NC5=O)coc4c3)n2)cc1Cl] | ¹H NMR (400 MHz, DMSO-d₆) δ 10.93 (s, 1H), 8.08 (d, J = 0.8 Hz, 1H), 8.03 (s, 1H), 7.81 (dd, J = 8.4, 1.2 Hz, 1H), 7.69 (d, J = 8.4 Hz, 1H), 7.39 (d, J = 2.0 Hz, 1H), 7.32 (s, 1H), 7.27 (dd, J = 8.0, 1.6 Hz, 1H), 7.09 (d, J = 8.4 Hz, 1H), 4.18 (dd, J = 12.4, 4.8 Hz, 1H), 4.07 (s, 2H), 3.82 (s, 3H), 2.72-2.80 (m, 1H), 2.57-2.62 (m, 1H), 2.29- 2.39 (m, 1H), 2.12-2.16 (m, 1H). LCMS: calc. for C₂₄H₁₉ClN₂O₄S: 466.1, found: [M + H]⁺ 467.1. |
| 384 | 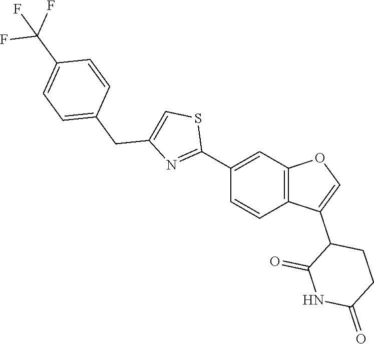 [SMILES: O=C1CCC(c2coc3cc(-c4nc(Cc5ccc(C(F)(F)F)cc5)cs4)ccc23)C(=O)N1] | ¹H NMR (400 MHz, DMSO-d₆) δ 10.92 (s, 1H), 8.08 (s, 1H), 8.03 (s, 1H), 7.81 (d, J = 8.4 Hz, 1H), 7.69 (d, J = 8.4 Hz, 3H), 7.57 (d, J = 8.0 Hz, 2H), 7.41 (s, 1H), 4.24 (s, 2H), 4.18 (dd, J = 12.0, 4.8 Hz, 1H), 2.69-2.84 (m, 1H), 2.55-2.65 (m, 1H), 2.26-2.41 (m, 1H), 2.08-2.21 (m, 1H). LCMS: calc. for C₂₄H₁₇F₃N₂O₃S: 470.1, found: [M + H]⁺ 471.2. |
| 385 | 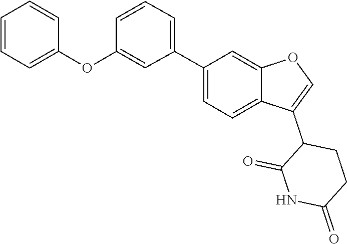 [SMILES: O=C1CCC(c2coc3cc(-c4cccc(Oc5ccccc5)c4)ccc23)C(=O)N1] | ¹H NMR (400 MHz, DMSO-d₆) δ 10.92 (s, 1H), 7.95 (s, 1H), 7.88 (d, J = 1.2 Hz, 1H), 7.65 (d, J = 8.0 Hz, 1H), 7.51-7.57 (m, 2H), 7.49 (d, J = 7.6 Hz, 1H), 7.42-7.47 (m, 1H), 7.38-7.42 (m, 2H), 7.13-7.18 (m, 1H), 7.09 (s, 1H), 7.07 (s, 1H), 6.95-6.99 (m, 1H), 4.17 (dd, J = 12.0, 4.8 Hz, 1H), 2.71-2.81 (m, 1H), 2.56-2.62 (m, 1H), 2.32-2.41 (m, 1H), 2.09-2.17 (m, 1H). LCMS: calc. for C₂₅H₁₉NO₄: 397.1, found: [M + H]⁺ 398.2. |
| 386 | 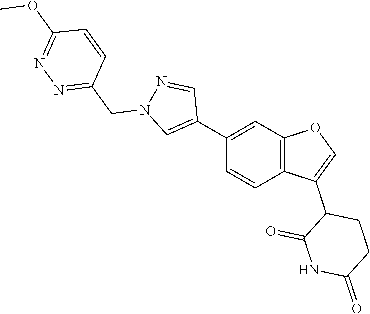 [SMILES: COc1ccc(Cn2cc(-c3ccc4c(C5CCC(=O)NC5=O)coc4c3)cn2)nn1] | ¹HNMR (400 MHz, DMSO-d₆) δ 10.90 (s, 1H), 8.39 (s, 1H), 8.02 (s, 1H), 7.86 (s, 1H), 7.81 (s, 1H), 7.54-7.58 (m, 1H), 7.47-7.52 (m, 2H), 7.23 (d, J = 9.2 Hz, 1H), 5.58 (s, 2H), 4.13 (dd, J = 11.6, 4.8 Hz, 1H), 4.02 (s, 3H), 2.73 (m, 1H), 2.62-2.69 (m, 1H), 2.33 (m, 1H), 2.09-2.17 (m, 1H). MS: (ESI) m/z calculated for C₂₂H₁₉N₅O₄ [M + H]⁺ 418.1, found 417.9. |
| 387 | 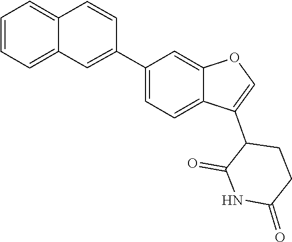 [SMILES: O=C1CCC(c2coc3cc(-c4ccc5ccccc5c4)ccc23)C(=O)N1] | ¹H NMR (400 MHz, DMSO-d₆) δ 10.93 (s, 1H), 8.30 (s, 1H), 7.91-8.07 (m, 6H), 7.69- 7.78 (m, 2H), 7.49-7.59 (m, 2H), 4.20 (dd, J = 12.0, 4.8 Hz, 1H), 2.71-2.84 (m, 1H), 2.59- 2.67 (m, 1H), 2.31-2.43 (m, 1H), 2.12-2.21 (m, 1H). LCMS: calc. for C₂₃H₁₇NO₃: 355.1, found: [M + H]⁺ 356.1 |

### Example 71:3-(5-fluoro-6-(1-(thiophen-3-ylmethyl)-1H-pyrazol-4-yl)benzofuran-3-yl)piperidine-2,6-dione

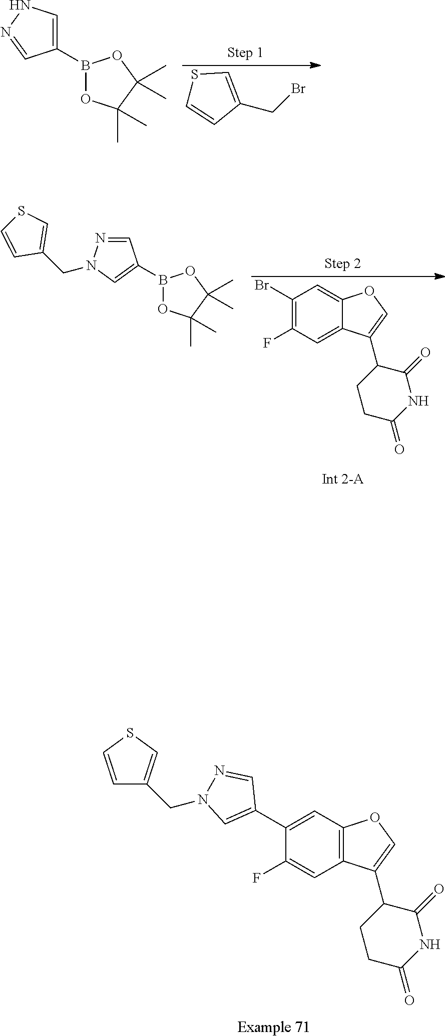

Step 1: To a solution of 4-(4,4,5,5-tetramethyl-1,3,2-dioxaborolan-2-yl)-1H-pyrazole (200 mg, 1.03 mmol, 1 eq) and 3-(bromomethyl)thiophene (219.00 mg, 1.24 mmol, 1.2 eq) in DMF (2 mL) was added Cs₂CO₃ (671.66 mg, 2.06 mmol, 2 eq) at 25° C. The mixture was stirred at 70° C. for 3 h. The reaction mixture was concentrated directly. The residue was purified by flash silica gel chromatography to give 4-(4,4,5,5-tetramethyl-1,3,2-dioxaborolan-2-yl)-1-(3-thienylmethyl)pyrazole (192 mg, 64.19% yield) as a colorless oil. ¹H NMR (400 MHZ, DMSO-d₆) δ 7.98 (s, 1H), 7.60-7.57 (m, 1H), 7.52-7.48 (m, 1H), 7.41-7.39 (m, 1H), 7.05-7.02 (m, 1H), 5.33-5.30 (m, 2H), 1.24 (s, 12H).

Step 2: To a solution of Int 2-A (30 mg, 91.99 μmol, 1 eq) in H₂O (0.5 mL) and dioxane (2 mL) was added 4-(4,4,5,5-tetramethyl-1,3,2-dioxaborolan-2-yl)-1-(3-thienylmethyl)pyrazole (32.03 mg, 110.39 μmol, 1.2 eq), K₃PO₄ (29.29 mg, 137.99 μmol, 1.5 eq) and XPhos Pd G3 (7.79 mg, 9.20 μmol, 0.1 eq) at 20° C. The mixture was stirred at 80° C. for 1 h under N₂. The reaction mixture was concentrated directly. The residue was purified by prep-SFC to give Example 71 (1.6 mg, 4.24% yield) as a white solid. ¹H NMR (400 MHZ, DMSO-d₆) § 10.91 (br s, 1H), 8.27 (d, J=2.4 Hz, 1H), 8.01 (s, 1H), 7.97-7.93 (m, 2H), 7.54-7.48 (m, 2H), 7.46 (d, J=4.0 Hz, 1H), 7.09 (dd, J=1.6, 5.2 Hz, 1H), 5.37 (s, 2H), 4.11 (dd, J=4.8, 12.0 Hz, 1H), 2.78-2.68 (m, 1H), 2.61-2.54 (m, 1H), 2.41-2.30 (m, 1H), 2.14-2.06 (m, 1H). LCMS: calc. for C₂₁H₁₆FN₃₀₃S: 409.1, found: [M+H]⁺410.0.

### Example 72: 3-[5-fluoro-6-[1-[(6-methoxy-3-pyridyl)methyl]pyrazol-4-yl]benzofuran-3-yl]piperidine-2,6-dione

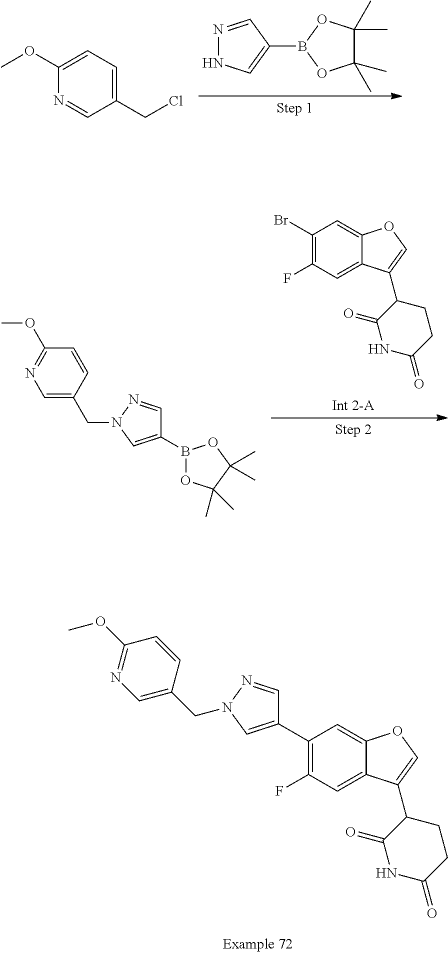

Step 1: To a solution of 4-(4,4,5,5-tetramethyl-1,3,2-dioxaborolan-2-yl)-1H-pyrazole (300 mg, 1.55 mmol, 1 eq) and 5-(chloromethyl)-2-methoxy-pyridine (292.39 mg, 1.86 mmol, 1.2 eq) in DMF (4 mL) was added Cs₂CO₃ (1.01 g, 3.09 mmol, 2 eq) at 20° C. The mixture was stirred at 80° C. for 3 h. The reaction mixture was concentrated. Water (20 mL) was added to the residue. The resulting mixture was extracted with EtOAc (20 mL×3). The combined organic phase was washed with brine (20 mL), dried over anhydrous Na₂SO₄, filtered and concentrated. The residue was purified by prep-HPLC to give 2-methoxy-5-[[4-(4,4,5,5-tetramethyl-1,3,2-dioxaborolan-2-yl)pyrazol-1-yl]methyl]pyridine (40 mg, 7.39% yield) as a yellow oil.

Step 2: To a solution of 2-methoxy-5-[[4-(4,4,5,5-tetramethyl-1,3,2-dioxaborolan-2-yl)pyrazol-1-yl]methyl]pyridine (39.95 mg, 114.07 μmol, 90% purity, 1.2 eq) in dioxane (2 mL) and H₂O (0.5 mL) was added Int 2-A (31 mg, 95.06 μmol, 1 eq), XPhos Pd G3 (8.05 mg, 9.51 μmol, 0.1 eq) and K₃PO₄ (40.36 mg, 190.12 μmol, 2 eq). The mixture was stirred under N₂ at 80° C. for 1 h. The reaction mixture was concentrated. The residue was purified by prep-HPLC to give Example 72 (1.8 mg, 4.10% yield) as a white solid. ¹H NMR (400 MHZ, DMSO-d₆) § 10.97-10.84 (m, 1H), 8.36-8.28 (m, 1H), 8.25-8.18 (m, 1H), 8.03-7.98 (m, 1H), 7.96-7.92 (m, 2H), 7.69 (dd, J=2.4, 8.4 Hz, 1H), 7.50 (d, J=12.0 Hz, 1H), 6.81 (d, J=8.0 Hz, 1H), 5.33 (s, 2H), 4.16-4.08 (m, 1H), 3.83 (s, 3H), 2.78-2.69 (m, 1H), 2.60-2.55 (m, 1H), 2.41-2.35 (m, 1H), 2.14-2.07 (m, 1H). 1° F. NMR (376 MHz, DMSO-d₆)₈-121.78. LCMS: calc. for C₂₃H₁₉FN₄O₄:434.1, found: [M+H]⁺435.3.

### Example 73: 3-[6-[1-[[4-(difluoromethoxy)phenyl]methyl]pyrazol-4-yl]-5-fluoro-benzofuran-3-yl]piperidine-2,6-dione

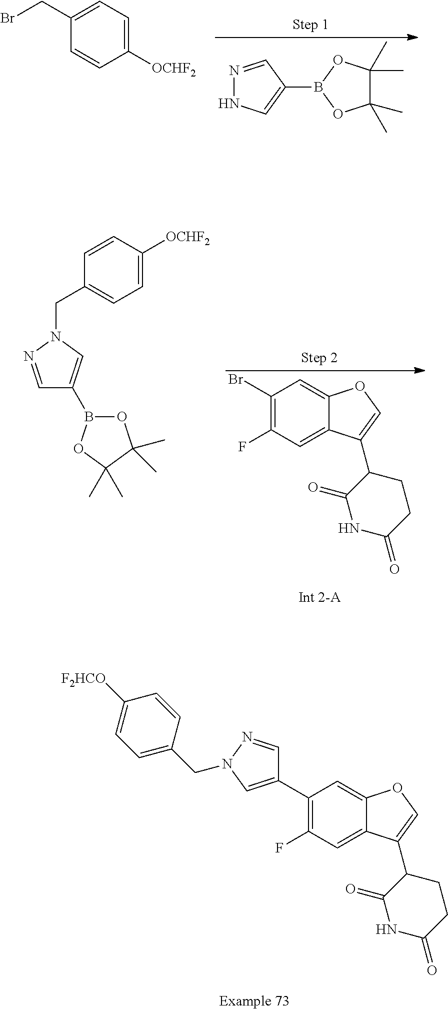 [SMILES: FC(F)Oc1ccc(CBr)cc1 ; CC1(C)OB(c2cn[nH]c2)OC1(C)C ; CC1(C)OB(c2cnn(Cc3ccc(OC(F)F)cc3)c2)OC1(C)C ; O=C1CCC(c2coc3cc(Br)c(F)cc23)C(=O)N1 ; O=C1CCC(c2coc3cc(-c4cnn(Cc5ccc(OC(F)F)cc5)c4)c(F)cc23)C(=O)N1]

Step 1: To a solution of 4-(4,4,5,5-tetramethyl-1,3,2-dioxaborolan-2-yl)-1H-pyrazole (300 mg, 1.55 mmol, 1 eq) and 1-(bromomethyl)-4-(difluoromethoxy)benzene (439.78 mg, 1.86 mmol, 1.2 eq) in DMF (3 mL) was added Cs₂CO₃ (1.01 g, 3.09 mmol, 2 eq) at 20° C. The mixture was stirred at 80° C. for 3 h. The reaction mixture was concentrated. Water (20 mL) was added to the residue. The resulting mixture was extracted with EtOAc (20 mL×3). The combined organic phase was washed with brine (20 mL), dried over anhydrous Na₂SO₄, filtered and concentrated. The residue was purified by flash silica gel chromatography to give 1-[[4-(difluoromethoxy)phenyl]methyl]-4-(4,4,5,5-tetramethyl-1,3,2-dioxaborolan-2-yl)pyrazole (260 mg, 48.02% yield) as a colorless oil.

Step 2: To a solution of Int 2-A (30 mg, 91.99 μmol, 1 eq) in dioxane (2 mL) and H₂O (0.5 mL) was added 1-[4-(difluoromethoxy)phenyl]methyl]-4-(4,4,5,5-tetramethyl-1,3,2-dioxaborolan-2-yl)pyrazole (38.65 mg, 110.39 μmol, 1.2 eq), XPhos Pd G3 (7.79 mg, 9.20 μmol, 0.1 eq) and K₃PO₄ (39.05 mg, 183.98 μmol, 2 eq). The mixture was stirred under N₂ at 80° C. for 1 h. The reaction mixture was concentrated directly. The residue was purified by prep-HPLC to give Example 73 (2.2 mg, 5.09% yield) as a white solid. ¹H NMR (400 MHz, DMSO-d₆) δ 10.91 (s, 1H), 8.32 (d, J=2.4 Hz, 1H), 8.02 (s, 1H), 7.96-7.93 (m, 2H), 7.50 (d, J=12.0 Hz, 1H), 7.40-7.35 (m, 2H), 7.21-7.15 (m, 2H), 5.38 (s, 2H), 4.11 (dd, J=4.8, 12.0 Hz, 1H), 2.78-2.66 (m, 2H), 2.57-2.54 (m, 1H), 2.35 (dd, J=4.0, 12.0 Hz, 1H), 2.14-2.08 (m, 1H). 1° F. NMR (376 MHZ, DMSO-d₆)₈-121.73, −81.95. LCMS: calc. for C₂₅H₁₈F₃N₃O₄:469.1, found: [M+H]⁺470.1.

### Example 74:3-[5-fluoro-6-[1-[(4-methylsulfonylphenyl)methyl]pyrazol-4-yl]benzofuran-3-yl]piperidine-2,6-dione

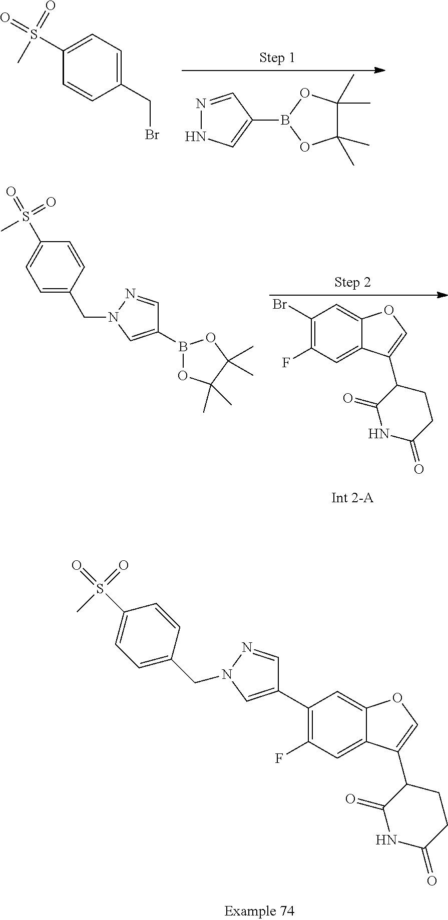

Step 1: To a solution of 4-(4,4,5,5-tetramethyl-1,3,2-dioxaborolan-2-yl)-1H-pyrazole (500 mg, 2.58 mmol, 1 eq) in DMF (5 mL) was added K₂CO₃ (712.26 mg, 5.15 mmol, 2 eq) and 1-(bromomethyl)-4-methylsulfonyl-benzene (770.34 mg, 3.09 mmol, 1.2 eq). The mixture was stirred at 80° C. for 3 h. The mixture was poured into water (50 mL) and extracted with EtOAc (50 mL×2). The combined organic layers were dried over Na₂SO₄, filtered and concentrated. The residue was purified by flash silica gel chromatography to give 1-[(4-methylsulfonylphenyl)methyl]-4-(4,4,5,5-tetramethyl-1,3,2-dioxaborolan-2-yl)pyrazole (510 mg, 54.63% yield) as a white solid.

Step 2: A mixture of 1-[(4-methylsulfonylphenyl)methyl]-4-(4,4,5,5-tetramethyl-1,3,2-dioxaborolan-2-yl)pyrazole (39.99 mg, 110.39 μmol, 1.2 eq), Int 2-A (30 mg, 91.99 μmol, 1 eq), K₃PO₄ (39.05 mg, 183.98 μmol, 2 eq), XPhos Pd G3 (7.79 mg, 9.20 μmol, 0.1 eq) in H₂O (0.75 mL) and dioxane (3 mL) was degassed and purged with N₂ for 3 times, and then the mixture was stirred at 80° C. for 2 h under N₂ atmosphere. The reaction mixture was concentrated directly. The residue was purified by prep-HPLC and dried by lyophilization to give Example 74 (16.6 mg, 37.10% yield) as a white solid. ¹H NMR (400 MHZ, DMSO-d₆) δ 10.91 (s, 1H), 8.39 (d, J=2.4 Hz, 1H), 8.07 (s, 1H), 7.98-7.94 (m, 2H), 7.92 (d, J=8.0 Hz, 2H), 7.55-7.49 (m, 3H), 5.53 (s, 2H), 4.12 (dd, J=4.8, 12.0 Hz, 1H), 3.19 (s, 3H), 2.78-2.68 (m, 1H), 2.59 (d, J=4.0 Hz, 1H), 2.39-2.33 (m, 1H), 2.16-2.06 (m, 1H). ¹⁹F NMR (376 MHZ, DMSO-d₆)₈-121.6. LCMS: calc. for C₂₄H₂OFN₃₀₅S: 481.1, found: [M+H]⁺482.3.

### Example 75:3-[5-fluoro-6-[1-[[4-(2-methoxyethyl)phenyl]methyl]pyrazol-4-yl]benzofuran-3-yl]piperidine-2,6-dione

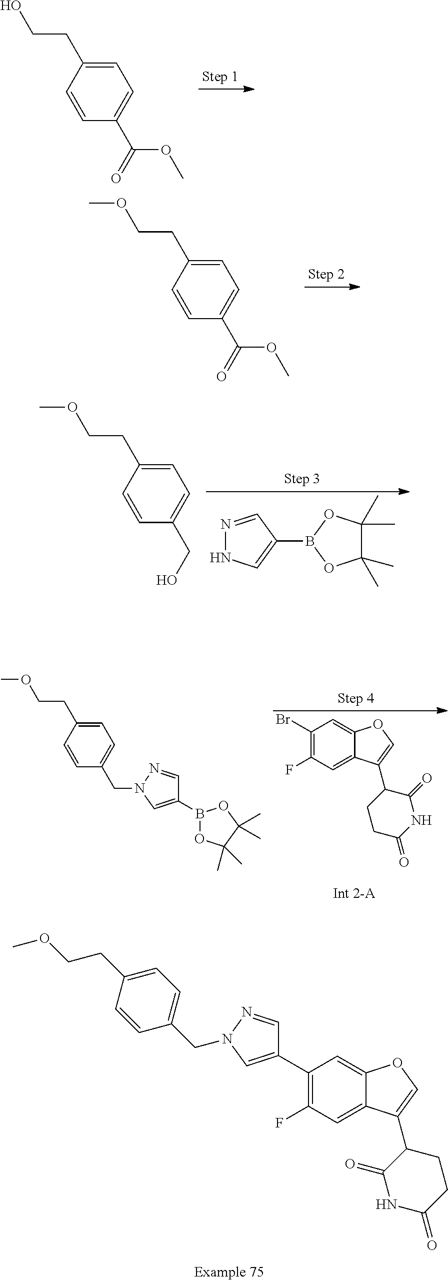

Step 1: To a solution of NaH (88.78 mg, 2.22 mmol, 60% purity, 2 eq) and methyl 4-(2-hydroxyethyl)benzoate (200 mg, 1.11 mmol, 1 eq) in THF (4 mL), degassed and purged with N₂ for 3 times, Mel (157.53 mg, 1.11 mmol, 1 eq) was added. The mixture was stirred at 25° C. for 1 h under N₂ atmosphere. The reaction was quenched with MeOH (5 mL), and the resulting mixture was filtered. The filtrate was concentrated directly to give methyl 4-(2-methoxyethyl)benzoate (301 mg, crude) as a yellow oil. ¹H NMR (400 MHz, CD₃OD) δ 7.91 (d, J=8.0 Hz, 2H), 7.24 (d, J=8.0 Hz, 2H), 3.85 (s, 3H), 3.57 (t, J=6.8 Hz, 2H), 3.30 (s, 3H), 2.88 (t, J=6.8 Hz, 2H).

Step 2: To a solution of methyl 4-(2-methoxyethyl)benzoate (200 mg, 1.03 mmol, 1 eq) in THF (5 mL) was added LAH (46.90 mg, 1.24 mmol, 1.2 eq) at 0° C. . . . The mixture was stirred at 25° C. for 16 h under N₂ atmosphere. The reaction was quenched with water (5 mL). The reaction mixture was concentrated. The resulting mixture was extracted with EtOAc (20 mL×3). The combined organic phase was washed with brine (30 mL), dried over anhydrous Na₂SO₄, filtered and concentrated. The residue was purified by flash silica gel chromatography to give [4-(2-methoxyethyl)phenyl]methanol (70 mg, 40.90% yield) as a yellow oil. ¹H NMR (400 MHZ, CDCl₃) δ 7.32-7.29 (m, 2H), 7.26-7.24 (m, 2H), 4.68-4.66 (m, 2H), 3.64-3.58 (m, 2H), 3.38-3.34 (m, 3H), 2.93-2.89 (m, 2H).

Step 3: A mixture of [4-(2-methoxyethyl)phenyl]methanol (70 mg, 421.14 μmol, 1 eq), 4-(4,4,5,5-tetramethyl-1,3,2-dioxaborolan-2-yl)-1H-pyrazole (81.72 mg, 421.14 μmol, 1 eq) in Tol. (4 mL) was sonicated for 10 min. Tributylphosphine (85.20 mg, 421.14 μmol, 1 eq), TMAD (108.77 mg, 631.71 μmol, 1.5 eq) were added and the reaction mixture was stirred at 25° C. for 16 h. The mixture was diluted with H₂O (20 mL), then extracted with EtOAc (15 mL×2). The organic layers were washed with brine (30 mL), dried over Na₂SO₄, filtered and concentrated under reduced pressure to give a residue. The residue was purified by flash silica gel chromatography to give 1-[[4-(2-methoxyethyl)phenyl]methyl]-4-(4,4,5,5-tetramethyl-1,3,2-dioxaborolan-2-yl)pyrazole (50 mg, 34.69% yield) as a colorless oil. ¹H NMR (400 MHZ, CDCl₃) δ 7.82 (s, 1H), 7.66 (s, 1H), 7.33-7.29 (m, 1H), 7.25-7.22 (m, 1 H), 7.21-7.15 (m, 2H), 5.29-5.28 (m, 2H), 4.68 (s, 1H), 3.63-3.56 (m, 3H), 3.36 (s, 2H), 3.36-3.34 (m, 2H), 2.92-2.84 (m, 3H), 1.33-1.29 (m, 12H).

Step 4: To a solution of 1-(4-(2-methoxyethyl)benzyl)-4-(4,4,5,5-tetramethyl-1,3,2-dioxaborolan-2-yl)-1H-pyrazole (50 mg, 146.10 μmol, 1 eq) and Int 2-A (47.64 mg, 146.10 μmol, 1 eq) in dioxane (2 mL) and H₂O (2 mL) was added K₃PO₄ (46.52 mg, 219.14 μmol, 1.5 eq) and XPhos Pd G3 (12.37 mg, 14.61 μmol, 0.1 eq). The mixture was stirred at 80° C. for 1.5 h. The reaction mixture was concentrated, and the residue was purified by prep-HPLC to give Example 75 (1 mg, 1.45% yield) as a white solid. ¹H NMR (400 MHZ, DMSO-d₆) § 10.89 (s, 1H), 8.29 (d, J=2.4 Hz, 1H), 8.00 (s, 1H), 7.96-7.91 (m, 1H), 7.49 (d, J=12.0 Hz, 1H), 7.21 (s, 4H), 5.34 (s, 2H), 4.13-4.09 (m, 1H), 3.52-3.48 (m, 1H), 3.21 (s, 3H), 2.77 (t, J=6.8, 4.0 Hz, 2H), 2.75-2.66 (m, 1H), 2.62-2.54 (m, 2H), 2.34-2.33 (m, 1H), 2.12-2.11 (m, 1H). LCMS: calc. for C₂₆H₂₄FN₃O₄:461.1, found: [M+H]⁺462.0.

### Example 218:3-[6-[1-[[5-(cyclopropylmethoxy)-2-pyridyl]methyl]pyrazol-4-yl]-5-fluoro-benzofuran-3-yl]piperidine-2,6-dione

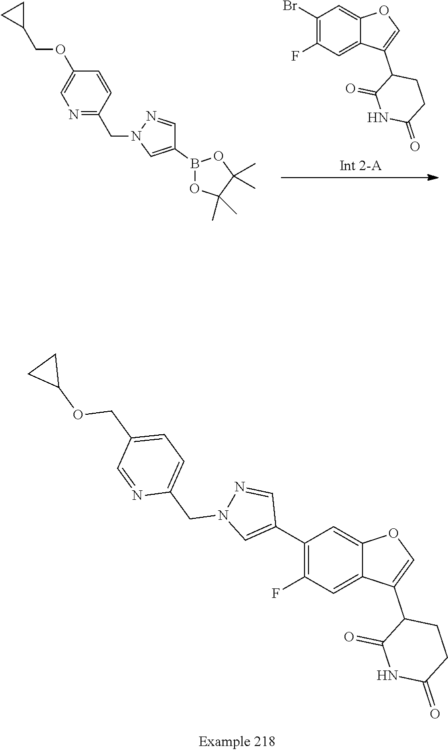 [SMILES: CC1(C)OB(c2cnn(Cc3ccc(OCC4CC4)cn3)c2)OC1(C)C ; O=C1CCC(c2coc3cc(Br)c(F)cc23)C(=O)N1 ; O=C1CCC(c2coc3cc(-c4cnn(Cc5ccc(COC6CC6)cn5)c4)c(F)cc23)C(=O)N1]

To a stirred solution of Int 2-A (50 mg, 153.32 μmol, 1 eq) in dioxane (2 mL) and H₂O (0.5 mL) was added 5-(cyclopropylmethoxy)-2-[[4-(4,4,5,5-tetramethyl-1,3,2-dioxaborolan-2-yl)pyrazol-1-yl]methyl]pyridine (65.36 mg, 183.98 μmol, 1.2 eq), XPhos Pd G3 (12.98 mg, 15.33 μmol, 0.1 eq), K₃PO₄ (48.82 mg, 229.98 μmol, 1.5 eq). The mixture was stirred under N₂ at 80° C. for 1 h. The reaction mixture was concentrated. The residue was purified by prep-HPLC to give Example 218 (8 mg, 11.00% yield) as a white solid. ¹H NMR (400 MHz, DMSO-d₆) § 10.91 (br s, 1H), 8.29 (d, J=2.4 Hz, 1H), 8.23 (d, J=2.8 Hz, 1H), 8.02 (d, J=3.6 Hz, 1H), 7.97-7.93 (m, 2H), 7.69-7.63 (m, 1H), 7.50 (d, J=12.0 Hz, 1H), 7.36 (dd, J=2.4, 8.8 Hz, 1H), 5.40 (s, 2H), 4.15-4.09 (m, 1H), 3.87 (d, J=8.0 Hz, 2H), 2.76-2.68 (m, 1H), 2.60-2.55 (m, 1H), 2.40-2.33 (m, 1H), 2.14-2.06 (m, 1H), 1.26-1.18 (m, 1H), 0.59-0.52 (m, 2H), 0.35-0.26 (m, 2H). ¹⁹F NMR (376 MHz, DMSO-d₆)₈-115.662, −121.708. LCMS: calc. for C₂₆H₂₃FN₄O₄:474.2, found: [M+H]⁺475.2.

### Example 219:3-[5-fluoro-6-[1-[(6-morpholinopyridazin-3-yl)methyl]pyrazol-4-yl]benzofuran-3-yl]piperidine-2,6-dione

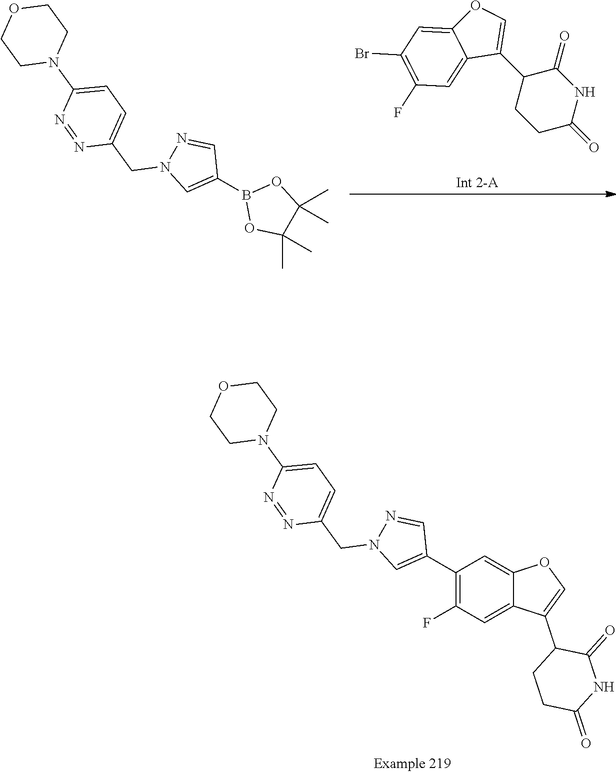 [SMILES: CC1(C)OB(c2cnn(Cc3ccc(N4CCOCC4)nn3)c2)OC1(C)C ; O=C1CCC(c2coc3cc(Br)c(F)cc23)C(=O)N1 ; O=C1CCC(c2coc3cc(-c4cnn(Cc5ccc(N6CCOCC6)nn5)c4)c(F)cc23)C(=O)N1]

A mixture of 4-[6-[4-(4,4,5,5-tetramethyl-1,3,2-dioxaborolan-2-yl)pyrazol-1-yl]methyl]pyridazin-3-yl]morpholine (59.76 mg, 160.98 μmol, 1.5 eq), Int 2-A (35 mg, 107.32 μmol, 1 eq), K₃PO₄ (45.56 mg, 214.65 μmol, 2 eq), XPhos Pd G3 (9.08 mg, 10.73 μmol, 0.1 eq) in dioxane (2 mL) and H₂O (0.5 mL) was degassed and purged with N₂ for 3 times, and then the mixture was stirred at 80° C. for 1 h under N₂ atmosphere. The reaction mixture was concentrated directly. The residue was purified by prep-HPLC to give Example 219 (3.8 mg, 6.90 μmol, 6.42% yield) as a white solid. 1HNMR (400 MHZ, DMSO-d₆) δ 11.03-10.80 (m, 1H), 8.34 (d, J=2.4 Hz, 1H), 8.03 (s, 1H), 7.98-7.84 (m, 2H), 7.51 (d, J=11.2 Hz, 1H), 7.37-7.24 (m, 2H), 5.52 (s, 2H), 4.17-4.03 (m, 1H), 3.73-3.68 (m, 4H), 3.55-3.50 (m, 4H), 2.80-2.68 (m, 1H), 2.62-2.52 (m, 1H), 2.41-2.29 (m, 1H), 2.11 (td, J=4.0, 12.8 Hz, 1H), ¹⁹F NMR (376 MHz, DMSO-d₆)₈-121.71. LCMS: calc. for C₂₅H₂₃FN₆O₄:490.2, found: [M+H]⁺491.2.

### Example 388:3-[5-fluoro-6-[2-[(4-methoxyphenyl)methyl]-1H-imidazol-5-yl]benzofuran-3-yl]piperidine-2,6-dione

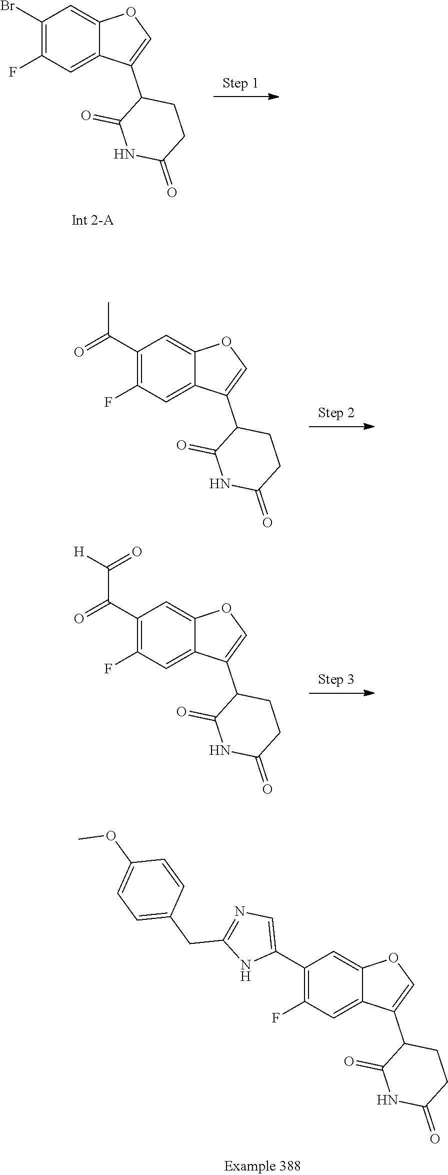 [SMILES: O=C1CCC(c2coc3cc(Br)c(F)cc23)C(=O)N1 ; CC(=O)c1cc2occ(C3CCC(=O)NC3=O)c2cc1F ; O=CC(=O)c1cc2occ(C3CCC(=O)NC3=O)c2cc1F ; COc1ccc(Cc2ncc(-c3cc4occ(C5CCC(=O)NC5=O)c4cc3F)[nH]2)cc1]

Step 1: To a stirred suspension of Int 2-A (300 mg, 919.91 μmol, 1 eq) in dioxane (8 mL) was added tributyl(1-ethoxyvinyl) stannane (1.16 g, 3.21 mmol, 1.08 mL, 3.49 eq) and Pd(PPh3)₂Cl₂ (64.57 mg, 91.99 μmol, 0.1 eq). The mixture was purged with nitrogen and heated at 90° C. for 16 h. The mixture was cooled to 20° C., 2 N aq. HCl (2 M, 689.94 μL, 1.5 eq) was added and the dark suspension heated at 45° C. for 30 min. The mixture was quenched by KF (300 mg) and the dioxane was evaporated under vacuum, the residue was dissolved in DCM (50 mL), and saturated aq. sodium bicarbonate was added until pH=4.0. The organic layer was washed with brine, dried over magnesiumsulfate, filtered, and concentrated to afford a crude compound. The residue was purified by flash silica gel chromatography Compound 3-(6-acetyl-5-fluoro-benzofuran-3-yl)piperidine-2,6-dione (200 mg, 691.42 μmol, 75.16% yield) was obtained as a light yellow solid. MS: (ESI) m/z calculated for C₁₅H₁₂FNO₄ [M+H]⁻290.0, found 290.0.

Step 2: To a solution of 3-(6-acetyl-5-fluoro-benzofuran-3-yl)piperidine-2,6-dione (200 mg, 691.42 μmol, 1 eq) in DMSO (3 mL) was added I₂ (175.49 mg, 691.42 μmol, 139.28 μL, 1 eq). The mixture was stirred at 110° C. for 2 h under N₂. LCMS indicated desired MS was detected. The reaction mixture was concentrated. The residue was purified by prep-HPLC. Compound 2-[3-(2,6-dioxo-3-piperidyl)-5-fluoro-benzofuran-6-yl]-2-oxo-acetaldehyde (100 mg, 329.77 μmol, 47.69% yield) was obtained as a white solid. MS: (ESI) m/z calculated for C₁₅H₁₀FNO₅ [M+H]⁺304.1, found 303.9.

Step 3: To a mixture of 2-[3-(2,6-dioxo-3-piperidyl)-5-fluoro-benzofuran-6-yl]-2-oxo-acetaldehyde (20 mg, 65.95 μmol, 1 eq) in DMSO (3 mL) was added 2-(4-methoxyphenyl) acetaldehyde (10.90 mg, 72.55 μmol, 1.1 eq), NH₄OAc (25.42 mg, 329.77 μmol, 5 eq). The reaction mixture was stirred at 100° C. for 1 h. LCMS indicated desired MS was detected. The reaction mixture was concentrated. The residue was purified by prep-HPLC. Example 388 (1.6 mg, 3.47 μmol, 5.26% yield, 94% purity) was obtained as a white solid. ¹H NMR (400 MHZ, d₆-DMSO) δ 12.06 (br s, 1H), 10.90 (br s, 1H), 8.08 (d, J=4.0 Hz, 1H), 7.92 (s, 1H), 7.45 (d, J=12.0 Hz, 1H), 7.38 (br d, J=4.0 Hz, 1H), 7.22 (d, J=8.0 Hz, 2H), 6.88 (d, J=8.0 Hz, 2H), 4.13-4.09 (m, 1H), 3.97 (s, 2H), 3.71 (s, 3H), 2.79-2.67 (m, 1H), 2.62-2.55 (m, 1H), 2.39-2.32 (m, 1H), 2.13-2.10 (m, 1H). 1° F. NMR (376 MHZ, d₆-DMSO) δ-121.4. MS: (ESI) m/z calculated for C₂₄H₂OFN₃₀₄ [M+H]⁺434.1, found 434.1.

### Example 389:3-[5-fluoro-6-[2-[(4-methoxyphenyl)methyl]oxazol-5-yl]benzofuran-3-yl]piperidine-2,6-dione

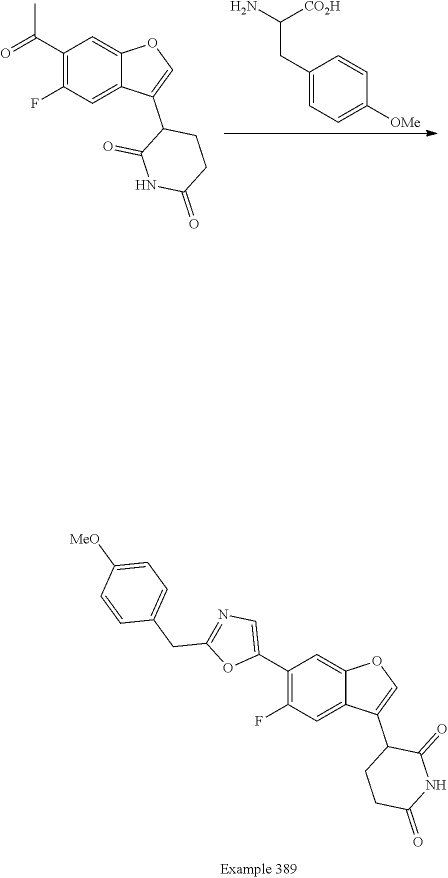 [SMILES: CC(=O)c1cc2occ(C3CCC(=O)NC3=O)c2cc1F ; COc1ccc(CC(N)C(=O)O)cc1 ; COc1ccc(Cc2ncc(-c3cc4occ(C5CCC(=O)NC5=O)c4cc3F)o2)cc1]

To a solution of 3-(6-acetyl-5-fluoro-benzofuran-3-yl)piperidine-2,6-dione (40 mg, 138.28 μmol, 1 eq) in DMSO (3 mL) was added I₂ (35.10 mg, 138.28 μmol, 27.86 μL, 1 eq). The mixture was stirred at 110° C. for 45 min under N₂, then 2-amino-3-(4-methoxyphenyl) propanoic acid (53.99 mg, 276.57 μmol, 2 eq) was added. The resulting mixture was stirred at 110° C. for 15 min. The reaction mixture was concentrated. The residue was purified by prep-HPLC. Example 389 (2.4 mg, 5.52 μmol, 4.00% yield, 100% purity) was obtained as a white solid. ¹H NMR (400 MHZ, d₆-DMSO) δ 10.92 (s, 1H), 8.06 (s, 1H), 7.85 (d, J=4.80 Hz, 1H), 7.65 (d, J=12.0 Hz, 1H), 7.44 (d, J=4.0 Hz, 1H), 7.30 (d, J=8.0 Hz, 2H), 6.91 (d, J=12.0 Hz, 2H), 4.17 (s, 2H), 3.73 (s, 3H), 2.77-2.66 (m, 1H), 2.60 (br d, J=4.0 Hz, 1H), 2.42-2.32 (m, 1H), 2.15-2.06 (m, 1H). ¹⁹F NMR (376 MHz, d₆-DMSO) δ-120.1. MS: (ESI) m/z calculated for C₂₄H₁₉FN₂₀₅ [M+H]⁺435.1, found 435.3.

### Example 390:3-[5-fluoro-6-[1-[(4-methoxyphenyl)methyl]-1,2,4-triazol-3-yl]benzofuran-3-yl]piperidine-2,6-dione

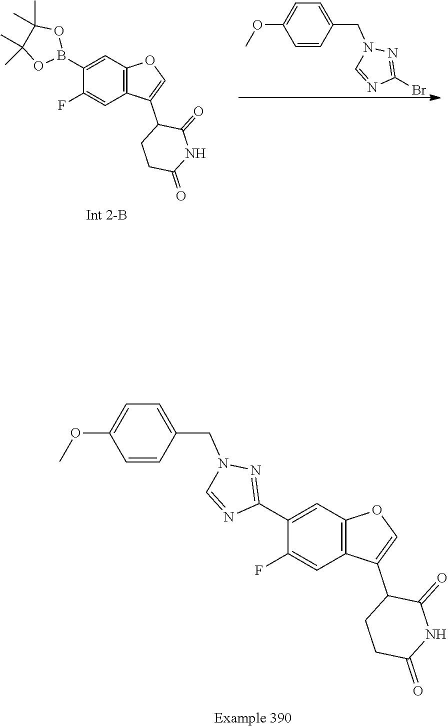 [SMILES: CC1(C)OB(c2cc3occ(C4CCC(=O)NC4=O)c3cc2F)OC1(C)C ; COc1ccc(Cn2cnc(Br)n2)cc1 ; COc1ccc(Cn2cnc(-c3cc4occ(C5CCC(=O)NC5=O)c4cc3F)n2)cc1]

To a mixture of 3-bromo-1-[(4-methoxyphenyl)methyl]-1,2,4-triazole (51.73 mg, 192.94 μmol, 1.2 eq), Int 2-B (60 mg, 160.78 μmol, 1 eq), K₃PO₄ (68.26 mg, 321.56 μmol, 2 eq), XPhos Pd G3 (13.61 mg, 16.08 μmol, 0.1 eq) in dioxane (4 mL) and H₂O (1 mL) was degassed and purged with N₂ for 3 times, and then the mixture was stirred at 80° C. for 2 hr under N₂ atmosphere. The reaction mixture was concentrated. The residue was purified by prep-HPLC. Example 390 (2.7 mg, 5.97 μmol, 3.71% yield, 96.07% purity) was obtained as a white solid. ¹H NMR (400 MHZ, do-DMSO) δ 10.92 (s, 1H), 8.73 (s, 1H), 8.11-8.02 (m, 2H), 7.57 (d, J=10.8 Hz, 1H), 7.33 (d, J=8.8 Hz, 2H), 6.94 (d, J=8.8 Hz, 2H), 5.41 (s, 2H), 4.15 (dd, J=4.8, 12.4 Hz, 1H), 3.73 (s, 3H), 2.74-2.65 (m, 1H), 2.60 (m, 1H), 2.40-2.31 (m, 1H), 2.16-2.07 (m, 1H). ¹⁹F NMR (376 MHz, do-DMSO) δ-119.4. MS: (ESI) m/z calculated for C₂₃H₁₉FN₄₀₄ [M+H]⁺435.1, found 435.2.

### Example 391:3-[5-fluoro-6-[3-(p-tolylmethyl)pyrrolidin-1-yl]benzofuran-3-yl]piperidine-2,6-dione

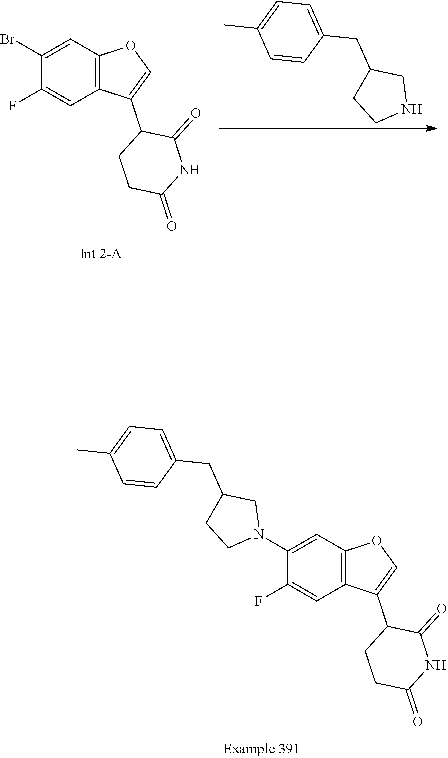 [SMILES: O=C1CCC(c2coc3cc(Br)c(F)cc23)C(=O)N1 ; Cc1ccc(CC2CCNC2)cc1 ; Cc1ccc(CC2CCN(c3cc4occ(C5CCC(=O)NC5=O)c4cc3F)C2)cc1]

To a mixture of Int 2-A (30 mg, 91.99 μmol, 1 eq), 3-(p-tolylmethyl)pyrrolidine (24.18 mg, 137.99 μmol, 1.5 eq), Cs₂CO₃ (119.89 mg, 367.97 μmol, 4 eq), Pd(OAc)₂ (4.13 mg, 18.40 μmol, 0.2 eq) in dioxane (3 mL) was added RuPhos (8.59 mg, 18.40 μmol, 0.2 eq) at 25° C. under N₂. The mixture was heated to 100° C. for 16 h under N₂. The reaction solution was concentrated. The residue was purified by prep-HPLC to give Example 391 (1.4 mg, 3.33 μmol, 3.62% yield) as a white solid. ¹H NMR (400 MHZ, DMSO-d₆) § 10.84 (s, 1H), 7.69 (s, 1H), 7.27 (br d, J=12.4 Hz, 1H), 7.17-7.03 (m, 4H), 6.86 (d, J=7.2 Hz, 1H), 4.00 (br d, J=4.8 Hz, 1H), 3.14-3.06 (m, 2H), 2.75-2.65 (m, 6H), 2.31 (br d, J=10.4 Hz, 1H), 2.27 (s, 3H), 2.13-2.04 (m, 1H), 2.02-1.93 (m, 2H), 1.64 (br dd, J=12.0, 8.0 Hz, 1H). 1° F. NMR (400 MHZ, DMSO-d₆)₈-131.50. MS: (ESI) m/z calculated for C₂₅H₂₅FN₂₀₃ [M+H]⁺421.1, found 421.4.

Examples 76-96, 220-237, and 392-427 were prepared following the similar procedure that was used for preparing Examples 71-75, starting from Int-2A and their corresponding boronic acid or ester intermediates, or Example 390, starting from Int-2B and their corresponding halides, or Example 391, starting from Int-2A and their corresponding amines with proper modifications. Characterizations of Examples 76-96, 220-237, and 392-427 are provided in Table 2.

TABLE 2

| Example | | |
|---|---|---|
| ID | Structure | NMR/MS |
| 76 | 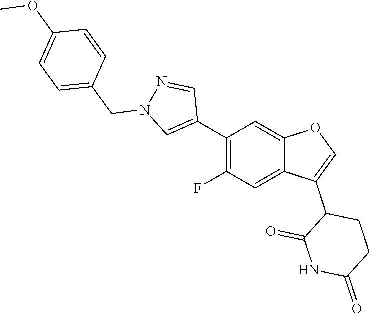 [SMILES: COc1ccc(Cn2cc(-c3cc4occ(C5CCC(=O)NC5=O)c4cc3F)cn2)cc1] | ¹H NMR (400 MHz, DMSO-d₆) δ 10.90 (br s, 1H), 8.26 (d, J = 2.4 Hz, 1H), 7.99 (s, 1H), 7.93 (t, J = 4.0 Hz, 2H), 7.49 (d, J = 12.0 Hz, 1H), 7.28 (d, J = 8.0 Hz, 2H), 6.91 (d, J = 8.4 Hz, 2H), 5.29 (s, 2H), 4.11 (dd, J = 4.8, 12.0 Hz, 1H), 3.72 (s, 3H), 2.81-2.63 (m, 1H), 2.62-2.53 (m, 1H), 2.35 (dq, J = 4.4, 12.0 Hz, 1H), 2.16-2.02 (m, 1H). ¹⁹F NMR (376 MHz, DMSO-d₆) δ −121.75. LCMS: calc. for C₂₄H₂₀FN₃O₄: 433.4, found: [M + H]⁺ 434.1 |
| 77 | 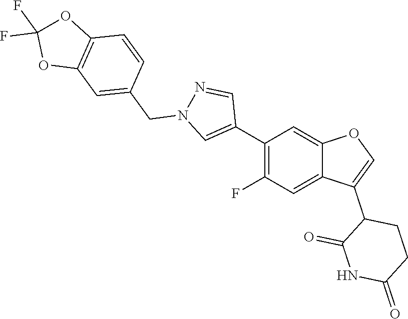 [SMILES: O=C1CCC(c2coc3cc(-c4cnn(Cc5ccc6c(c5)OC(F)(F)O6)c4)c(F)cc23)C(=O)N1] | ¹H NMR (400 MHz, d₆-DMSO) δ (s, 1H), 8.34 (d, J = 1.2 Hz, 1H), 8.03 (s, 1H), 7.98-7.90 (m, 2H), 7.51 (br d, J = 11.2 Hz, 1H), 7.44-7.36 (m, 2H), 7.19 (br d, J = 8.4 Hz, 1H), 5.40 (s, 2H), 4.12 (br dd, J = 4.8, 12.0 Hz, 1H), 2.82-2.66 (m, 1H), 2.58 (br d, J = 18.0 Hz, 1H), 2.35 (br dd, J = 3.6, 12.8 Hz, 1H), 2.14-2.04 (m, 1H). ¹⁹F NMR (376 MHz, d₆-DMSO) δ −49.0, −121.7. LCMS: calc. for C₂₄H₁₆F₃N₃O₅: 483.1, found: [M + H]⁺ 484.3 |
| 78 | 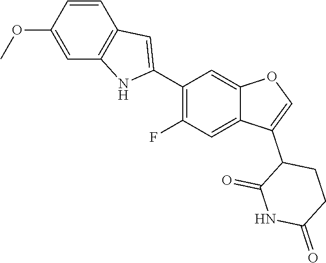 [SMILES: COc1ccc2cc(-c3cc4occ(C5CCC(=O)NC5=O)c4cc3F)[nH]c2c1] | ¹H NMR (400 MHz, DMSO-d₆) δ 11.34 (s, 1H), 10.92 (s, 1H), 8.06 (d, J = 6.4 Hz, 1H), 8.01 (s, 1H), 7.58 (d, J = 12.0 Hz, 1H), 7.45 (d, J = 8.8 Hz, 1H), 6.93 (d, J = 2.0 Hz, 1H), 6.88 (s, 1H), 6.69 (dd, J = 8.8, 2.4 Hz, 1H), 4.15 (dd, J = 12.0, 4.8 Hz, 1H), 3.80 (s, 3H), 2.70-2.80 (m, 1H), 2.56- 2.63 (m, 1H), 2.35-2.44 (m, 1H), 2.09-2.17 (m, 1H). LCMS: calc. for C₂₂H₁₇FN₂O₄: 392.1, found: [M + H]⁺ 393.1. |
| 79 | 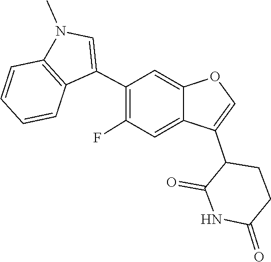 [SMILES: Cn1cc(-c2cc3occ(C4CCC(=O)NC4=O)c3cc2F)c2ccccc21] | ¹H NMR (400 MHz, DMSO-d₆) δ 10.90 (s, 1H), 7.97 (s, 1H), 7.80 (d, J = 6.0 Hz, 1H), 7.73 (d, J = 8.0 Hz, 1H), 7.67 (d, J = 2.0 Hz, 1H), 7.50-7.58 (m, 2H), 7.25 (t, J = 7.2 Hz, 1H), 7.11-7.17 (m, 1H), 4.15 (dd, J = 12.0, 4.8 Hz, 1H), 3.88 (s, 3H), 2.70-2.80 (m, 1H), 2.57-2.63 (m, 1H), 2.39 (dd, J = 12.8, 4.4 Hz, 1H), 2.10-2.18 (m, 1H). LCMS: calc. for C₂₂H₁₇FN₂O₃: 376.1, found: [M + H]⁺ 377.1 |
| 80 | 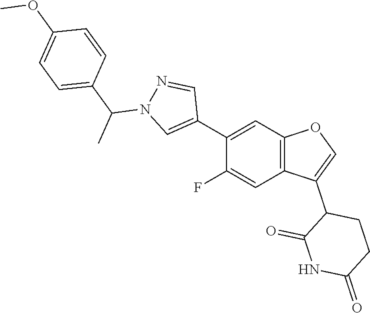 [SMILES: COc1ccc(C(C)n2cc(-c3cc4occ(C5CCC(=O)NC5=O)c4cc3F)cn2)cc1] | ¹H NMR (400 MHz, d₆-DMSO) δ 10.91 (s, 1H), 8.28 (d, J = 1.6 Hz, 1H), 8.01-7.90 (m, 3H), 7.49 (d, J = 11.2 Hz, 1H), 7.27 (d, J = 8.8 Hz, 2H), 6.90 (d, J = 8.8 Hz, 2H), 5.61 (q, J = 7.2 Hz, 1H), 4.11 (dd, J = 5.2, 12.4 Hz, 1H), 3.72 (s, 3H), 2.74- 2.65 (m, 1H), 2.57-2.53 (m, 1H), 2.39-2.30 (m, 1H), 2.14-2.06 (m, 1H), 1.82 (d, J = 6.8 Hz, 3H). ¹⁹F NMR (376 MHz, d₆-DMSO) δ −121.7. LCMS: calc. for C₂₅H₂₂FN₃O₄: 447.2, found: [M + H]⁺ 448.4 |
| 81 | 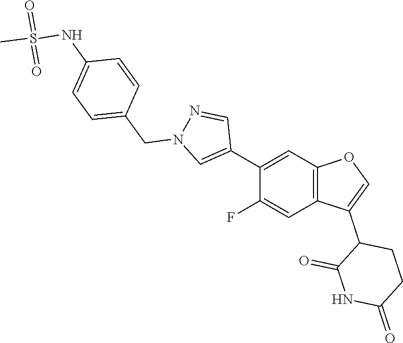 [SMILES: CS(=O)(=O)Nc1ccc(Cn2cc(-c3cc4occ(C5CCC(=O)NC5=O)c4cc3F)cn2)cc1] | ¹H NMR (400 MHz, DMSO-d₆) δ 10.90 (s, 1H), 9.86-9.63 (m, 1H), 8.30 (d, J = 2.4 Hz, 1H), 8.02 (s, 1H), 7.97-7.83 (m, 2H), 7.50 (d, J = 11.2 Hz, 1H), 7.30 (d, J = 8.4 Hz, 2H), 7.18 (d, J = 8.4 Hz, 2H), 5.33 (s, 2H), 4.12 (dd, J = 4.8, 12.0 Hz, 1H), 2.97 (s, 3H), 2.78-2.66 (m, 1H), 2.62-2.55 (m, 1H), 2.41-2.27 (m, 1H), 2.13-2.08 (m, 1H), ¹⁹F NMR (376 MHz, DMSO- d₆) δ −121.73. LCMS: calc. for C₂₄H₂₁FN₄O₅S: 496.1, found: [M + H]⁺ 497.3 |
| 82 | 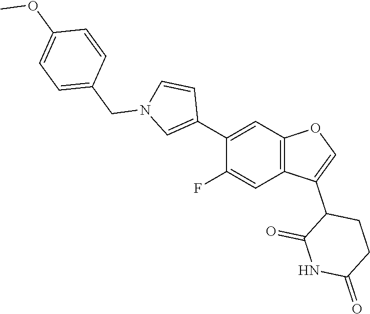 [SMILES: COc1ccc(Cn2ccc(-c3cc4occ(C5CCC(=O)NC5=O)c4cc3F)c2)cc1] | ¹H NMR (400 MHz, DMSO-d₆) δ 10.93-10.86 (m, 1H), 7.88 (s, 1H), 7.78 (d, J = 8.0 Hz, 1H), 7.40 (d, J = 12.0 Hz, 1H), 7.33 (d, J = 4.0 Hz, 1H), 7.26-7.23 (m, 2H), 6.92-6.89 (m, 3H), 6.58- 6.56 (m, 1H), 5.07-5.05 (m, 2H), 4.12-4.06 (m, 1H), 3.73 (s, 3H), 2.72-2.67 (m, 1H), 2.59 (br s, 1H), 2.36-2.32 (m, 1H), 2.13-2.07 (m, 1H). LCMS: calc. for C₂₅H₂₁FN₂O₄: 432.1, found: [M + H]⁺ 433.3 |
| 83 | 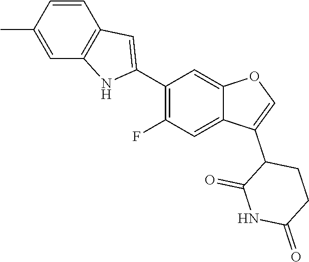 [SMILES: Cc1ccc2cc(-c3cc4occ(C5CCC(=O)NC5=O)c4cc3F)[nH]c2c1] | ¹H NMR (400 MHz, DMSO-d₆) δ 11.36 (s, 1H), 10.94 (s, 1H), 8.10 (d, J = 6.0 Hz, 1H), 8.02 (s, 1H), 7.59 (d, J = 11.6 Hz, 1H), 7.45 (d, J = 8.0 Hz, 1H), 7.23 (s, 1H), 6.89 (s, 1H), 6.86 (d, J = 8.0 Hz, 1H), 4.15 (dd, J = 12.0, 4.8 Hz, 1H), 2.70-2.80 (m, 1H), 2.58-2.64 (m, 1H), 2.41 (s, 3H), 2.31-2.39 (m, 1H), 2.08-2.17 (m, 1H). LCMS: calc. for C₂₂H₁₇FN₂O₃: 376.1, found: [M + H]⁺ 377.2. |
| 84 | 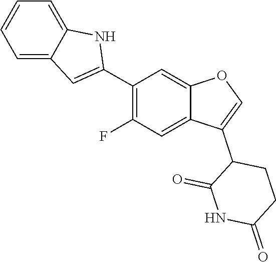 [SMILES: O=C1CCC(c2coc3cc(-c4cc5ccccc5[nH]4)c(F)cc23)C(=O)N1] | ¹H NMR (400 MHz, DMSO-d₆) δ 11.53 (s, 1H), 10.93 (s, 1H), 8.12 (d, J = 6.0 Hz, 1H), 8.03 (s, 1H), 7.55-7.64 (m, 2H), 7.45 (d, J = 7.6 Hz, 1H), 7.11-7.17 (m, 1H), 7.00-7.05 (m, 1H), 6.96 (s, 1H), 4.16 (dd, J = 12.0, 4.8 Hz, 1H), 2.70-2.81 (m, 1H), 2.60-2.64 (m, 1H), 2.40-2.44 (m, 1H), 2.09- 2.17 (m, 1H). LCMS: calc. for C₂₁H₁₅FN₂O₃: 362.1, found: [M + H]⁺ 363.1. |
| 85 | 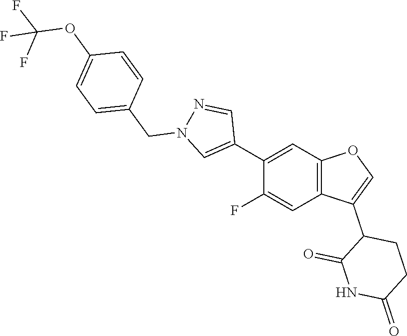 [SMILES: O=C1CCC(c2coc3cc(-c4cnn(Cc5ccc(OC(F)(F)F)cc5)c4)c(F)cc23)C(=O)N1] | ¹H NMR (400 MHz, d₆-DMSO) δ 10.91 (s, 1H), 8.36 (d, J = 2.4 Hz, 1H), 8.05 (s, 1H), 8.00-7.93 (m, 2H), 7.51 (d, J = 11.2 Hz, 1H), 7.45-7.34 (m, 4H), 5.44 (s, 2H), 4.12 (dd, J = 4.8, 12.0 Hz, 1H), 2.79-2.69 (m, 1H), 2.59 (m, 1H), 2.35 (m, 1H), 2.16-2.05 (m, 1H). ¹⁹F NMR (376 MHz, DMSO- d₆) δ −56.82, −121.72. LCMS: calc. for C₂₄H₁₇F₄N₃O₄: 487.1, found: [M + H]⁺ 488.2 |
| 86 | 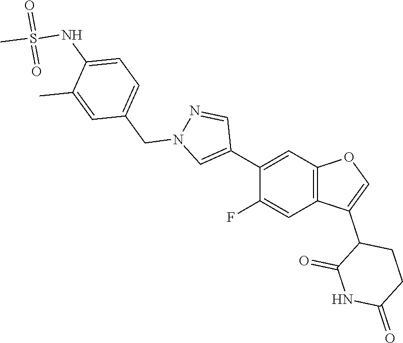 [SMILES: Cc1cc(Cn2cc(-c3cc4occ(C5CCC(=O)NC5=O)c4cc3F)cn2)ccc1NS(C)(=O)=O] | ¹H NMR (400 MHz, DMSO-d₆) δ 10.87 (s, 1H), 9.16-8.96 (m, 1H), 8.29 (d, J = 2.0 Hz, 1H), 7.99 (s, 1H), 7.93-7.88 (m, 2H), 7.46 (d, J = 10.8 Hz, 1H), 7.25-7.19 (m, 2H), 7.12 (br d, J = 8.4 Hz, 1H), 5.31 (s, 2H), 4.10 (br dd, J = 12.4, 4.8 Hz, 1H), 2.94 (s, 3H), 2.79-2.67 (m, 1H), 2.61-2.53 (m, 1H), 2.32 (br dd, J = 12.4, 4.8 Hz, 1H), 2.26 (s, 3H), 2.15-2.05 (m, 1H). ¹⁹F NMR (376 MHz, DMSO-d₆) δ −121.63. LCMS: calc. for C₂₅H₂₃FN₄O₅S: 510.1, found: [M + H]⁺ 510.9 |
| 87 | 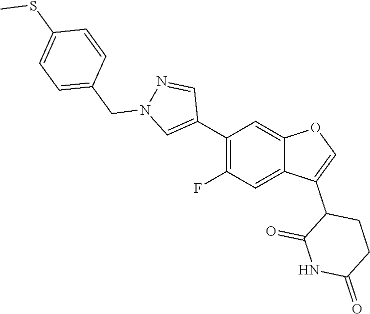 [SMILES: CSc1ccc(Cn2cc(-c3cc4occ(C5CCC(=O)NC5=O)c4cc3F)cn2)cc1] | ¹H NMR (400 MHz, DMSO-d₆) δ 10.88 (s, 1H), 8.28 (d, J = 2.4 Hz, 1H), 8.00 (s, 1H), 7.93 (t, J = 3.2 Hz, 2H), 7.49 (d, J = 11.2 Hz, 1H), 7.25 (s, 4H), 5.33 (s, 2H), 4.11 (dd, J = 12.0, 4.8 Hz, 1H), 2.65-2.71 (m, 1H), 2.59 ( m, 1H), 2.45 (s, 3H), 2.34 (m, 1H), 2.06-2.15 (m, 1H). ¹⁹F NMR (376 MHz, DMSO-d₆) δ −112.75. LCMS: calc. for C₂₄H₂₀FN₃O₃S: 449.1, found: [M + H]⁺ 450.0 |
| 88 | 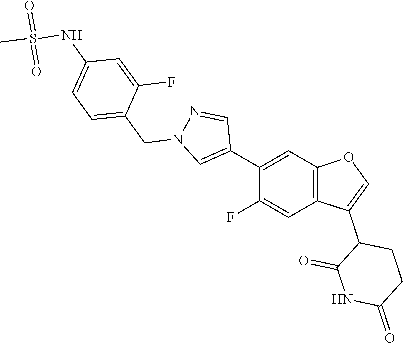 [SMILES: CS(=O)(=O)Nc1ccc(Cn2cc(-c3cc4occ(C5CCC(=O)NC5=O)c4cc3F)cn2)c(F)c1] | ¹H NMR (400 MHz, DMSO-d₆) δ 10.90 (s, 1H), 10.08 (br s, 1H), 8.26 (d, J = 2.0 Hz, 1H), 8.01 (s, 1H), 7.99-7.91 (m, 2H), 7.50 (d, J = 11.2 Hz, 1H), 7.30 (t, J = 8.4 Hz, 1H), 7.09-6.92 (m, 2H), 5.37 (s, 2H), 4.12 (dd, J = 12.4, 4.2 Hz, 1H), 3.04 (s, 3H), 2.79-2.67 (m, 1H), 2.64-2.55 (m, 1H), 2.36-2.34 (m, 1H), 2.18-2.03 (m, 1H). ¹⁹F NMR (376 MHz, -DMSO-d₆) δ −121.75, −116.26. LCMS: calc. for C₂₄H₂₀F₂N₄O₅S: 514.1, found: [M + H]⁺ 515.4 |
| 89 | 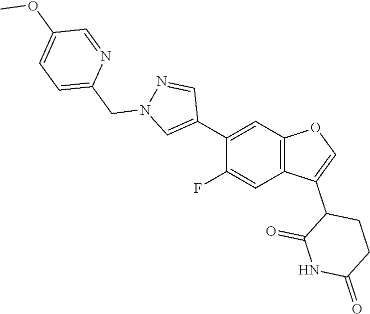 [SMILES: COc1ccc(Cn2cc(-c3cc4occ(C5CCC(=O)NC5=O)c4cc3F)cn2)nc1] | ¹H NMR (400 MHz, d₆-DMSO) δ 8.30 (d, J = 2.4 Hz, 1H), 8.26 (d, J = 2.8 Hz, 1H), 8.02 (s, 1H), 7.99-7.92 (m, 2H), 7.51 (d, J = 12.0 Hz, 1H), 7.39 (dd, J = 3.2, 8.8 Hz, 1H), 7.20 (d, J = 8.8 Hz, 1H), 5.42 (s, 2H), 4.17-4.09 (dd, J = 4.8, 12.4 Hz, 1H), 3.81 (s, 3H), 2.80-2.68 (m, 1H), 2.63- 2.54 (m, 1H), 2.36 (dq, J = 4.4, 12.4 Hz, 1H), 2.17- 2.06 (m, 1H). ¹F NMR (376 MHz, d₆-DMSO) δ −121.7. LCMS: calc. for C₂₃H₁₉FN₄O₄: 434.1, found: [M + H]⁺ 435.1 |
| 90 | 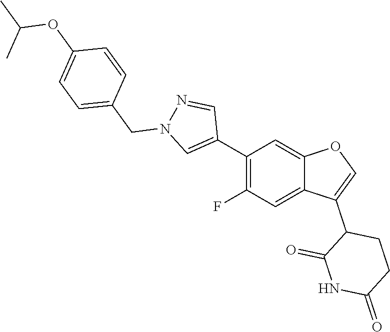 [SMILES: CC(C)Oc1ccc(Cn2cc(-c3cc4occ(C5CCC(=O)NC5=O)c4cc3F)cn2)cc1] | ¹H NMR (400 MHz, DMSO-d₆) δ 11.24-10.87 (m, 1H), 8.26 (d, J = 2.4 Hz, 1H), 8.10 (d, J = 7.6 Hz, 1H), 8.00 (s, 1H), 7.94-7.93 (m, 1H), 7.47 (d, J = 5.4 Hz, 1H), 7.37 (dt, J = 1.2, 7.6 Hz, 1H), 7.23 (s, 1H), 7.17-7.11 (m, 1H), 6.89-6.87 (m, 1H), 5.28 (s, 2H), 4.58 (td, J = 6.1, 12.0 Hz, 1H), 4.11 (dd, J = 4.8, 12.0 Hz, 1H), 2.77-2.68 (m, 1H), 2.63-2.54 (m, 1H), 2.35 (dd, J = 4.4, 12.8 Hz, 1H), 2.12 (br s, 1H), 1.28-1.19 (m, 6H). ¹F NMR (376 MHz, DMSO-d₆) δ −121.765. LCMS: calc. for C₂₆H₂₄FN₃O₄: 461.1, found: [M + H]⁺ 462.2 |
| 91 | 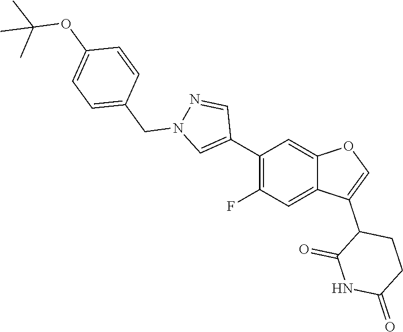 [SMILES: CC(C)(C)Oc1ccc(Cn2cc(-c3cc4occ(C5CCC(=O)NC5=O)c4cc3F)cn2)cc1] | ¹H NMR (400 MHz, DMSO-d₆) δ 10.90 (s, 1H), 8.30 (d, J = 4.0 Hz, 1H), 8.02 (s, 1H), 7.96-7.93 (m, 2H), 7.50 (d, J = 8.0 Hz, 1H), 7.22 (d, J = 8.0 Hz, 2H), 6.96 (s, 1H), 6.95-6.94 (m, 1H), 5.33 (s, 2H), 4.11 (dd, J = 8.0, 12.0 Hz, 1H), 2.71 (br d, J = 12.0 Hz, 1H), 2.61-2.54 (m, 1H), 2.38-2.31 (m, 1H), 2.14-2.06 (m, 1H), 1.28-1.26 (m, 9H). LCMS: calc. for C₂₇H₂₆FN₃O₄: 475.1, found: [M + H]⁺ 476.4 |
| 92 | 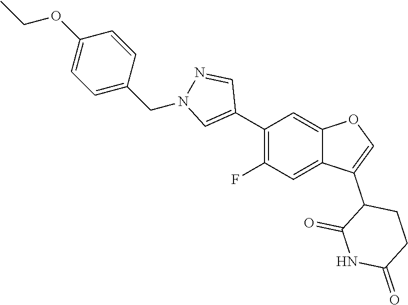 [SMILES: CCOc1ccc(Cn2cc(-c3cc4occ(C5CCC(=O)NC5=O)c4cc3F)cn2)cc1] | ¹H NMR (400 MHz, DMSO-d₆) δ 10.90 (br s, 1H), 8.26 (d, J = 2.4 Hz, 1H), 8.00 (s, 1H), 7.96- 7.91 (m, 2H), 7.50 (d, J = 12.0 Hz, 1H), 7.26 (d, J = 8.8 Hz, 2H), 6.94-6.86 (m, 2H), 5.29 (s, 2H), 4.12 (dd, J = 4.8, 12.0 Hz, 1H), 4.00 (q, J = 8.0 Hz, 2H), 2.80-2.67 (m, 1H), 2.63-2.53 (m, 1H), 2.35 (dd, J = 4.4, 12.0 Hz, 1H), 2.15-2.06 (m, 1H), 1.30 (t, J = 8.0 Hz, 3H). ¹⁹F NMR (376 MHz, DMSO-d₆) δ −121.769. LCMS: calc. for C₂₅H₂₂FN₃O₄: 447.1, found: [M + H]⁺ 448.2 |
| 93 | 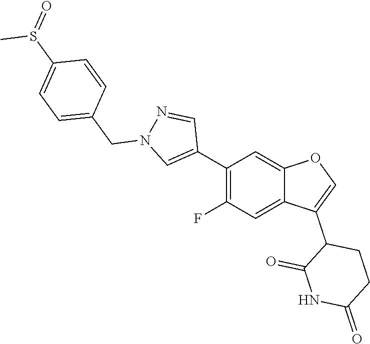 [SMILES: CS(=O)c1ccc(Cn2cc(-c3cc4occ(C5CCC(=O)NC5=O)c4cc3F)cn2)cc1] | ¹H NMR (400 MHz, DMSO-d₆) δ 10.90 (s, 1H), 8.37 (d, J = 2.4 Hz, 1H), 8.05 (s, 1H), 7.98-7.93 (m, 2H), 7.67 (d, J = 8.4 Hz, 2H), 7.53-7.46 (m, 3H), 5.48 (s, 2H), 4.16-4.08 (m, 1H), 2.72 (s, 4H), 2.60 (s, 1H), 2.33 (br s, 1H), 2.17-2.03 (m, 1H), ¹⁹F NMR (376 MHz, DMSO-d₆) δ −121.70. LCMS: calc. for C₂₄H₂₀FN₃O₄S: 465.1, found: [M + H]⁺ 466.3 |
| 94 | 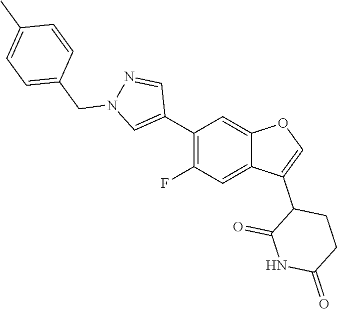 [SMILES: Cc1ccc(Cn2cc(-c3cc4occ(C5CCC(=O)NC5=O)c4cc3F)cn2)cc1] | ¹HNMR (400 MHz, DMSO-d₆) δ 10.89 (s, 1H), 8.27 (d, J = 2.0 Hz, 1H), 8.00 (s, 1H), 7.94 (t, J = 3.2 Hz, 2H), 7.49 (d, J = 11.6 Hz, 1H), 7.13-7.23 (m, 4H), 5.33 (s, 2H), 4.11 (dd, J = 12.0, 4.8 Hz, 1H), 2.67-2.78 (m, 1H), 2.54-2.62 (m, 1H), 2.35 (m, 1H), 2.28 (s, 3H), 2.06-2.16 (m, 1H). ¹⁹F NMR (376 MHz, DMSO-d₆) δ −112.76. LCMS: calc. for C₂₄H₂₀FN₃O₃: 417.1, found: [M + H]⁺ 418.0 |
| 95 | 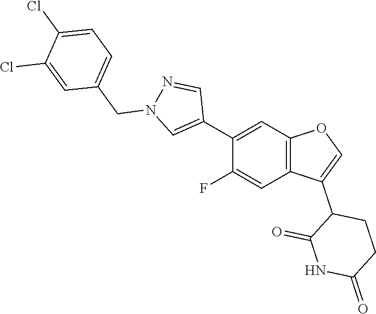 [SMILES: O=C1CCC(c2coc3cc(-c4cnn(Cc5ccc(Cl)c(Cl)c5)c4)c(F)cc23)C(=O)N1] | ¹H NMR (400 MHz, d₆-DMSO) 10.96-10.85 (m, 1H), 8.36 (d, J = 2.4 Hz, 1H), 8.06 (s, 1H), 7.99- 7.93 (m, 2H), 7.64 (d, J = 8 Hz, 1H), 7.59 (d, J = 1.9 Hz, 1H), 7.51 (d, J = 11.2 Hz, 1H), 7.28 (dd, J = 2.0, 8.2 Hz, 1H), 5.43-5.39 (m, 2H), 4.12 (dd, J = 4.8, 12.2 Hz, 1H), 2.78-2.67 (m, 1H), 2.62- 2.55 (m, 1H), 2.41-2.29 (m, 1H), 2.15-2.07 (m, 1H). LCMS: calc. for C₂₃H₁₆Cl₂FN₃O₃: 471.1, found: [M + H]⁺ 472.1 |
| 96 | 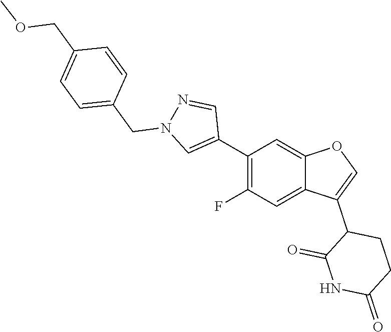 [SMILES: COCc1ccc(Cn2cc(-c3cc4occ(C5CCC(=O)NC5=O)c4cc3F)cn2)cc1] | ¹H NMR (400 MHz, DMSO- d₆) δ 10.90 (s, 1H), 8.33-8.29 (m, 1H), 8.03 (s, 1H), 7.97-7.93 (m, 2H), 7.51 (d, J = 12.0 Hz, 1H), 7.33-7.26 (m, 4H), 5.39 (s, 2H), 4.39 (s, 2H), 4.12 (dd, J = 4.8, 12.4 Hz, 1H), 3.27 (s, 3H), 2.80-2.67 (m, 1H), 2.63-2.54 (m, 1H), 2.36 (q, J = 4.4, 12.0 Hz, 1H), 2.16-2.07 (m, 1H). LCMS: calc. for C₂₅H₂₂FN₃O₄: 447.1, found: [M + H]⁺ 448.0 |
| 220 | 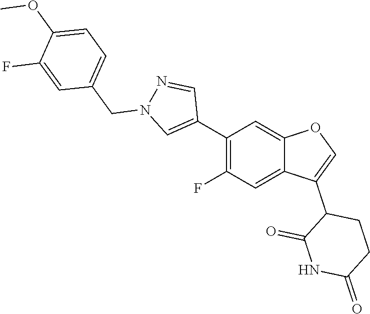 [SMILES: COc1ccc(Cn2cc(-c3cc4occ(C5CCC(=O)NC5=O)c4cc3F)cn2)cc1F] | ¹H NMR (400 MHz, DMSO-d₆) δ 10.90 (s, 1H), 8.29 (d, J = 2.4 Hz, 1H), 8.01 (s, 1H), 7.94 (t, J = 2.8 Hz, 2H), 7.50 (d, J = 12.0 Hz, 1H), 7.23-7.09 (m, 3H), 5.31 (s, 2H), 4.11 (dd, J = 4.8, 12.0 Hz, 1H), 3.81 (s, 3H), 2.78-2.66 (m, 1H), 2.57-2.52 (m, 1H), 2.37-2.31 (m, 1H), 2.15-2.05 (m, 1H). MS: (ESI) m/z calculated for C₂₄H₁₉F₂N₃O₄ [M + H]⁺ 452.1, found 452.3. |
| 221 | 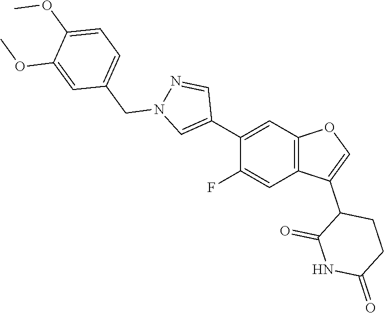 [SMILES: COc1ccc(Cn2cc(-c3cc4occ(C5CCC(=O)NC5=O)c4cc3F)cn2)cc1OC] | ¹HNMR (400 MHz, DMSO-d₆) δ 10.90 (s, 1H), 8.26 (d, J = 2.4 Hz, 1H), 8.00 (s, 1H), 7.95-7.93 (m, 2H), 7.51 (s, 1H), 7.00 (d, J = 2.0 Hz, 1H), 6.92 (s, 1H), 6.86 (s, 1H), 5.28 (s, 2H), 4.12 (s, 1H), 3.73 (d, J = 3.2 Hz, 6H), 2.76-2.70 (m, 1H), 2.60-2.55 (m, 1H), 2.35 (dd, J = 4.4, 12.8 Hz, 1H), 2.13-2.08 (m, 1H). ¹⁹FNMR (376 MHz, DMSO-d₆) δ −121.771. MS: (ESI) m/z calculated for C₂₅H₂₂FN₃O₅ [M + H]⁺ 464.1, found 464.3. |
| 222 | 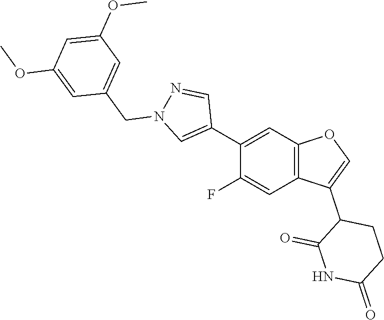 [SMILES: COc1cc(Cn2cc(-c3cc4occ(C5CCC(=O)NC5=O)c4cc3F)cn2)cc(OC)c1] | ¹H NMR (400 MHz, DMSO-d₆) δ 10.90 (s, 1H), 8.30 (d, J = 2.4 Hz, 1H), 8.02 (s, 1H), 7.97-7.90 (m, 2H), 7.50 (d, J = 11.2 Hz, 1H), 6.46-6.44 (m, 2H), 6.44-6.42 (m, 1H), 5.30 (s, 2H), 4.12 (dd, J = 4.8, 12.4 Hz, 1H), 3.72-3.70 (m, 1H), 3.71 (s, 5H), 2.77-2.69 (m, 1H), 2.58 (br d, J = 17.6 Hz, 1H), 2.43-2.34 (m, 1H), 2.12 (br d, J = 4.0 Hz, 1H). ¹⁹F NMR (376 MHz, DMSO-d₆) δ −121.7. MS: (ESI) m/z calculated for C₂₅H₂₂FN₃O₅ [M + H]⁺ 464.1, found 464.3. |
| 223 | 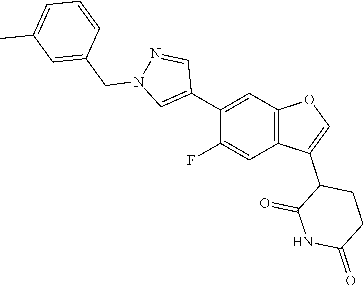 [SMILES: Cc1cccc(Cn2cc(-c3cc4occ(C5CCC(=O)NC5=O)c4cc3F)cn2)c1] | ¹H NMR (400 MH_{Z}, DMSO-d₆) δ 10.90 (s, 1 H), 8.30 (d, J = 2.0 Hz, 1 H), 8.02 (s, 1 H), 7.98-7.92 (m, 2 H), 7.50 (d, J = 11.2 Hz, 1 H), 7.21-7.27 (m, 1 H), 7.07-7.14 (m, 3 H), 5.34 (s, 2 H), 4.11 (dd, J = 12.4, 4.8 Hz, 1 H), 2.68-2.77 (m, 1 H), 2.60 (m, 1 H), 2.28 (s, 4 H), 2.06-2.15 (m, 1 H). ¹⁹FNMR (376 MHz, DMSO-d₆) δ −121.7. MS: (ESI) m/z calculated for C₂₄H₂₀FN₃O₃ [M + H]⁺ 418.1, found 418.1. |
| 224 | 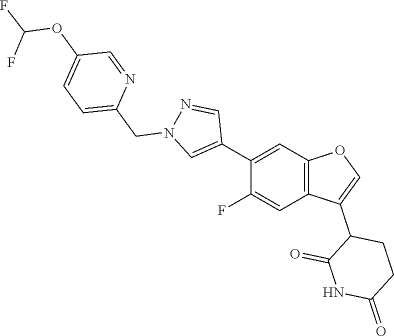 [SMILES: O=C1CCC(c2coc3cc(-c4cnn(Cc5ccc(OC(F)F)cn5)c4)c(F)cc23)C(=O)N1] | ¹H NMR (400 MHz, DMSO-d₆) δ 11.11-10.69 (m, 1H), 8.46 (d, J = 2.8 Hz, 1H), 8.35 (d, J = 2.4 Hz, 1H), 8.05 (s, 1H), 8.00-7.92 (m, 2H), 7.68 (dd, J = 2.8, 8.8 Hz, 1H), 7.56-7.09 (m, 3H), 5.51 (s, 2H), 4.12 (dd, J = 4.8, 12.4 Hz, 1H), 2.80- 2.67 (m, 1H), 2.58 (td, J = 3.6, 16.0 Hz, 1H), 2.36 (dq, J = 4.4, 12.8 Hz, 1H), 2.17-2.05 (m, 1H). ¹⁹F NMR (376 MHz, DMSO-d₆) δ −82.271, −121.684. MS: (ESI) m/z calculated for C₂₃H₁₇F₃N₄O₄ [M + H]⁺ 471.1, found 471.2. |
| 225 | 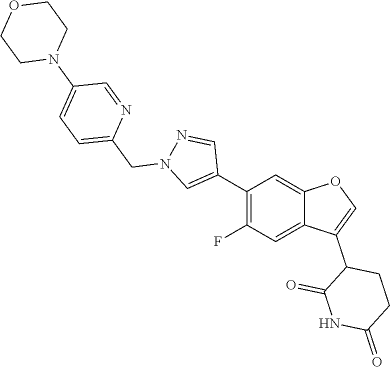 [SMILES: O=C1CCC(c2coc3cc(-c4cnn(Cc5ccc(N6CCOCC6)cn5)c4)c(F)cc23)C(=O)N1] | ¹H NMR (400 MHz, DMSO-d₆) δ 10.89 (s, 1H), 8.27-8.24 (m, 2H), 8.00 (s, 1H), 7.97-7.92 (m, 2H), 7.49 (d, J = 11.2 Hz, 1H), 7.33-7.30 (m, 1H), 7.11 (d, J = 8.8 Hz, 1H), 5.36 (s, 2H), 4.13- 4.09 (m, 1H), 3.75-3.70 (m, 4H), 3.16-3.12 (m, 4H), 2.78-2.68 (m, 1H), 2.61-2.53 (m, 1H), 2.40-2.29 (m, 1H), 2.15-2.06 (m, 1H). ¹⁹F NMR (376 MHz, DMSO-d₆) δ −121.74. MS: (ESI) m/z calculated for C₂₆H₂₄FN₅O₄ [M + H]⁺ 490.1, found 490.1. |
| 226 | 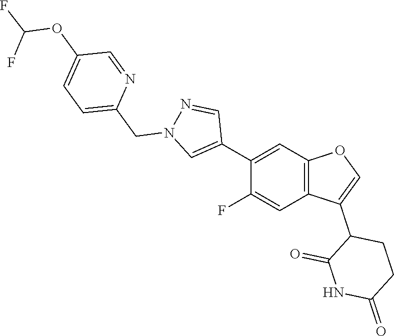 [SMILES: O=C1CCC(c2coc3cc(-c4cnn(Cc5ccc(OC(F)F)cn5)c4)c(F)cc23)C(=O)N1] | ¹H NMR (400 MHz, DMSO-d₆) δ 10.90 (s, 1H), 8.36 (d, J = 2.4 Hz, 1H), 8.29 (d, J = 2.0 Hz, 1H), 8.03 (s, 1H), 7.95 (t, J = 3.6 Hz, 2H), 7.91-7.65 (m, 2H), 7.56-7.47 (m, 1H), 7.09 (d, J = 8.4 Hz, 1H), 5.42 (s, 2H), 4.11 (dd, J = 4.8, 12.0 Hz, 1H), 2.80-2.67 (m, 1H), 2.63-2.55 (m, 1H), 2.44- 2.32 (m, 1H), 2.20-2.01 (m, 1H). |
| 227 | 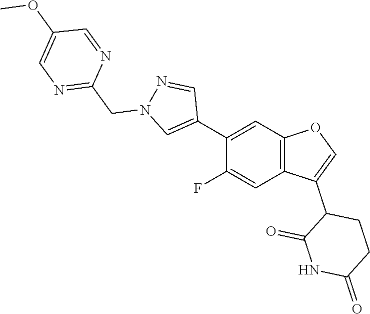 [SMILES: COc1cnc(Cn2cc(-c3cc4occ(C5CCC(=O)NC5=O)c4cc3F)cn2)nc1] | ¹H NMR (400 MHz, DMSO-d₆) δ 8.54 (s, 2H), 8.31 (d, J = 2.0 Hz, 1H), 8.00 (s, 1H), 7.98-7.93 (m, 2H), 7.51 (d, J = 12.0 Hz, 1H), 5.54 (s, 2H), 4.12 (dd, J = 4.0, 12.0 Hz, 1H), 3.89 (s, 3H), 2.80- 2.66 (m, 1H), 2.63-2.53 (m, 1H), 2.36 (d, J = 4.4, 12.0 Hz, 1H), 2.18-2.05 (m, 1H). ¹⁹F NMR (376 MHz, DMSO-d₆) δ −121.767. MS: (ESI) m/z calculated for C₂₂H₁₈N₅O₄ [M + H]⁺ 436.1, found 436.2. |
| 228 | 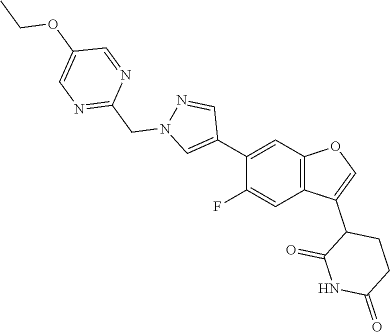 [SMILES: CCOc1cnc(Cn2cc(-c3cc4occ(C5CCC(=O)NC5=O)c4cc3F)cn2)nc1] | ¹H NMR (400 MHz, DMSO-d₆) δ 10.92 (s, 1H), 8.52 (s, 2H), 8.31 (d, J = 2.0 Hz, 1H), 8.00 (s, 1H), 7.98-7.94 (m, 2H), 7.51 (d, J = 12.0 Hz, 1H), 5.54 (s, 2H), 4.18 (q, J = 8.0 Hz, 2H), 4.13 (dd, J = 4.8, 12.0 Hz, 1H), 2.79-2.69 (m, 1H), 2.63- 2.55 (m, 1H), 2.37 (d, J = 4.0, 12.0 Hz, 1H), 2.15- 2.07 (m, 1H), 1.34 (t, J = 8.9 Hz, 3H). ¹⁹F NMR (376 MHz, DMSO-d₆) δ −121.702. MS: (ESI) m/z calculated for C₂₃H₂₀FN₅O₄ [M + H]⁺ 450.1, found 450.2. |
| 229 | 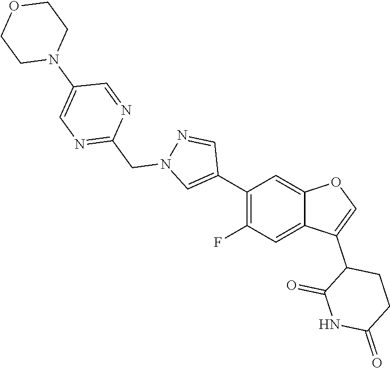 [SMILES: O=C1CCC(c2coc3cc(-c4cnn(Cc5ncc(N6CCOCC6)cn5)c4)c(F)cc23)C(=O)N1] | ¹H NMR (400 MHz, DMSO-d₆) δ 10.91 (br s, 1H), 8.48 (s, 2H), 8.28 (d, J = 2.8 Hz, 1H), 8.02- 7.92 (m, 3H), 7.51 (d, J = 11.2 Hz, 1H), 5.47 (s, 2H), 4.12 (dd, J = 4.8, 12.0 Hz, 1H), 3.77-3.68 (m, 4H), 3.25-3.19 (m, 4H), 2.79-2.67 (m, 1H), 2.63-2.55 (m, 1H), 2.43-2.30 (m, 1H), 2.11 (td, J = 4.4, 8.0 Hz, 1H), ¹⁹F NMR (376 MHz, DMSO- d₆) δ −121.722. MS: (ESI) m/z calculated for C₂₅H₂₃FN₆O₄ [M + H]⁺ 491.1, found 491.4. |
| 230 | 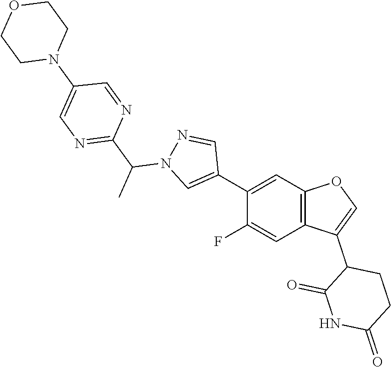 [SMILES: CC(c1ncc(N2CCOCC2)cn1)n1cc(-c2cc3occ(C4CCC(=O)NC4=O)c3cc2F)cn1] | ¹H NMR (400 MHz, DMSO-d₆) δ 10.94-10.88 (m, 1H), 8.52-8.44 (m, 2H), 8.32-8.27 (m, 1H), 8.01-7.90 (m, 3H), 7.50 (d, J = 12.0 Hz, 1H), 5.70 (q, J = 8.0 Hz, 1H), 4.12 (dd, J = 4.8, 12.4 Hz, 1H), 3.77-3.71 (m, 4H), 3.24-3.20 (m, 4H), 2.75- 2.66 (m, 1H), 2.61-2.55 (m, 1H), 2.39-2.31 (m, 1H), 2.15-2.07 (m, 1H), 1.86 (d, J = 7.2 Hz, 3H). ¹⁹F NMR (376 MHz, DMSO-d₆) δ −121.78. MS: (ESI) m/z calculated for C₂₆H₂₅FN₆O₄ [M + H]⁺ 505.2, found 505.2. |
| 231 | 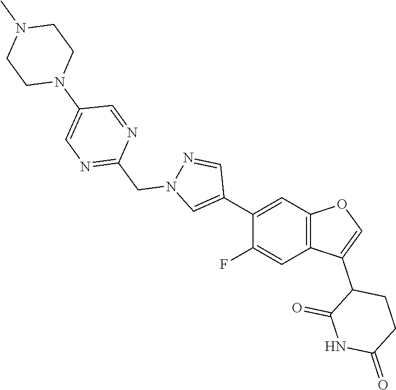 [SMILES: CN1CCN(c2cnc(Cn3cc(-c4cc5occ(C6CCC(=O)NC6=O)c5cc4F)cn3)nc2)CC1] | ¹H NMR (400 MHz, DMSO-d₆) δ 10.94-10.87 (m, 1H), 8.55 (s, 2H), 8.30 (d, J = 2.4 Hz, 1H), 8.02-7.91 (m, 3H), 7.55-7.47 (m, 1H), 5.49 (s, 2H), 4.12 (dd, J = 4.8, 12.0 Hz, 1H), 4.08-3.89 (m, 2H), 3.53-3.39 (m, 2H), 3.29-3.02 (m, 4H), 2.86-2.80 (m, 3H), 2.79-2.69 (m, 1H), 2.62- 2.55 (m, 1H), 2.41-2.31 (m, 1H), 2.14-2.07 (m, 1H). ¹⁹F NMR (376 MHz, DMSO-d₆) δ −121.72. MS: (ESI) m/z calculated for C₂₆H₂₆FN₇O₃ [M + H]⁺ 504.2, found 504.3. |
| 232 | 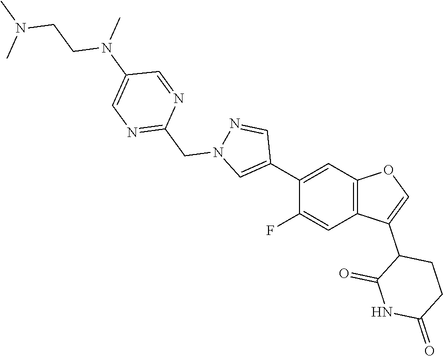 [SMILES: CN(C)CCN(C)c1cnc(Cn2cc(-c3cc4occ(C5CCC(=O)NC5=O)c4cc3F)cn2)nc1] | ¹H NMR (400 MHz, DMSO-d₆) δ 10.91 (s, 1H), 9.64 (s, 1H), 8.38-8.23 (m, 3H), 8.01-7.92 (m, 3H), 7.50 (d, J = 12.0 Hz, 1H), 5.45 (s, 2H), 4.12 (dd, J = 4.8, 12.0 Hz, 1H), 3.69 (d, J = 8.0 Hz, 2H), 3.25 (s, 2H), 2.96 (s, 3H), 2.82 (d, J = 2.4 Hz, 6H), 2.73 (d, J = 12.0, 16.0 Hz, 1H), 2.62-2.53 (m, 1H), 2.41-2.30 (m, 1H), 2.15-2.06 (m, 1H). ¹⁹F NMR (376 MHz, DMSO-d₆) δ −121.763, −74.092. MS: (ESI) m/z calculated for C₂₆H₂₈FN₇O₃ [M + H]⁺ 506.2, found 506.3. |
| 233 | 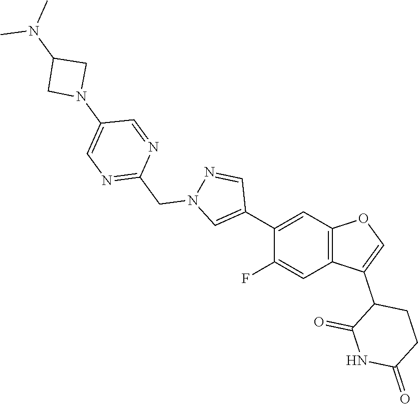 [SMILES: CN(C)C1CN(c2cnc(Cn3cc(-c4cc5occ(C6CCC(=O)NC6=O)c5cc4F)cn3)nc2)C1] | ¹H NMR (400 MHz, DMSO-d₆) δ 10.92 (s, 1H), 8.30 (s, 1H), 8.26 (d, J = 2.0 Hz, 1H), 8.02 (s, 2H), 7.97 (d, J = 3.6 Hz, 1H), 7.95-7.94 (m, 1H), 7.50 (d, J = 12.0 Hz, 1H), 5.43 (s, 2H), 4.12 (dd, J = 4.8, 12.0 Hz, 1H), 3.99 (t, J = 8.0 Hz, 2H), 3.69 (dd, J = 5.6, 8.0 Hz, 2H), 3.23 (d, J = 8.0, 12.0 Hz, 2H), 2.78-2.67 (m, 1H), 2.62-2.55 (m, 1H), 2.39- 2.33 (m, 1H), 2.09 (s, 6H). ¹⁹F NMR (376 MHz, DMSO-d₆) δ −121.725. MS: (ESI) m/z calculated for C₂₆H₂₆FN₇O₃ [M + H]⁺ 504.2, found 504.0. |
| 234 | 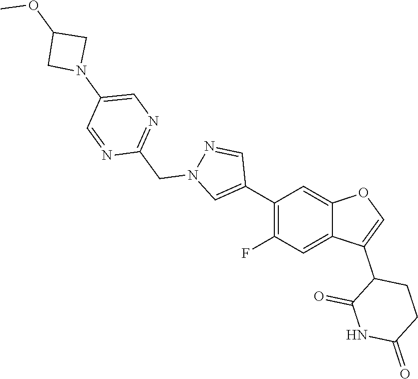 [SMILES: COC1CN(c2cnc(Cn3cc(-c4cc5occ(C6CCC(=O)NC6=O)c5cc4F)cn3)nc2)C1] | ¹H NMR (400 MHz, DMSO-d₆) δ 10.94-10.88 (m, 1H), 8.26 (d, J = 2.8 Hz, 1H), 8.03 (s, 2H), 8.00-7.92 (m, 3H), 7.50 (d, J = 11.2 Hz, 1H), 5.44 (s, 2H), 4.37-4.32 (m, 1H), 4.17-4.08 (m, 3H), 3.75-3.72 (m, 2H), 3.23 (s, 3H), 2.75-2.68 (m, 1H), 2.60 (br d, J = 4.0 Hz, 1H), 2.39-2.30 (m, 1H), 2.16-2.07 (m, 1H). ¹⁹F NMR (376 MHz, DMSO-d₆) δ −121.74. MS: (ESI) m/z calculated for C₂₅H₂₃FN₆O₄ [M + H]⁺ 491.1, found 491.1. |
| 235 | 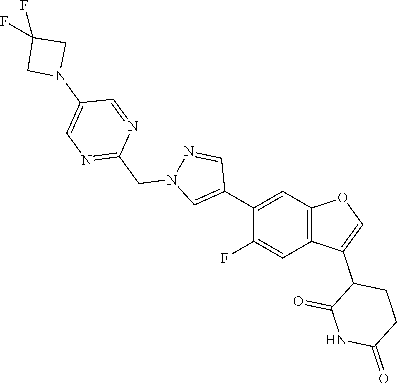 [SMILES: O=C1CCC(c2coc3cc(-c4cnn(Cc5ncc(N6CC(F)(F)C6)cn5)c4)c(F)cc23)C(=O)N1] | ¹H NMR (400 MHz, DMSO-d₆) δ 10.89 (s, 1 H), 8.53 (s, 1 H), 8.29-8.27 (m, 1 H), 8.18-8.15 (m, 2 H), 7.99-7.94 (m, 3 H), 7.53-7.48 (m, 1 H), 5.49-5.46 (m, 2 H), 4.46-4.38 (m, 4 H), 4.15- 4.09 (m, 1 H), 2.74-2.66 (m, 1 H), 2.62-2.55 (m, 1 H), 2.35-2.31 (m, 1 H), 2.15-2.07 (m, 1 H). MS: (ESI) m/z calculated for C₂₄H₁₉F₃N₆O₃ [M + H]⁺ 497.1 found 497.4. |
| 236 | 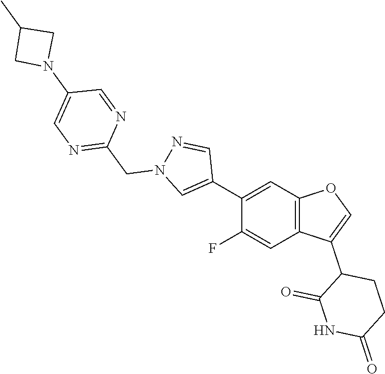 [SMILES: CC1CN(c2cnc(Cn3cc(-c4cc5occ(C6CCC(=O)NC6=O)c5cc4F)cn3)nc2)C1] | ¹H NMR (400 MHz, DMSO-d₆) δ 11.01-10.81 (m, 1H), 8.25 (d, J = 2.4 Hz, 1H), 8.04-7.88 (m, 5H), 7.50 (d, J = 11.2 Hz, 1H), 5.42 (s, 2H), 4.12 (dd, J = 4.8, 12.0 Hz, 1H), 4.05 (t, J = 7.6 Hz, 2H), 3.49 (dd, J = 6.0, 7.2 Hz, 2H), 2.84 (dt, J = 7.2, 13.6 Hz, 1H), 2.77-2.66 (m, 1H), 2.60 (br t, J = 4.0 Hz, 1H), 2.42-2.28 (m, 1H), 2.19-2.05 (m, 1H), 1.22 (d, J = 6.8 Hz, 3H). MS: (ESI) m/z calculated for C₂₅H₂₃FN₆O₃ [M + H]⁺ 475.1, found 475.2. |
| 237 | 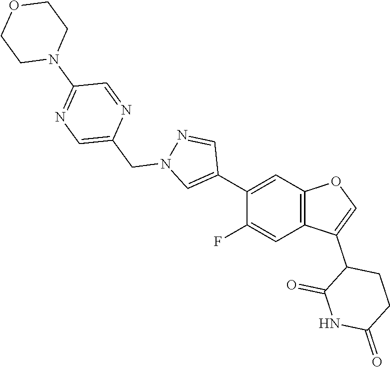 [SMILES: O=C1CCC(c2coc3cc(-c4cnn(Cc5cnc(N6CCOCC6)cn5)c4)c(F)cc23)C(=O)N1] | ¹HNMR (400 MHz, DMSO-d₆) δ 10.89 (s, 1H), 8.28 (d, J = 1.2 Hz, 1H), 8.27 (d, J = 2.8 Hz, 1H), 8.16-8.13 (m, 1H), 7.98 (s, 1H), 7.95-7.93 (m, 2H), 7.50 (d, J = 11.2 Hz, 1H), 5.35 (s, 2H), 4.13- 4.09 (m, 1H), 3.71-3.67 (m, 4H), 3.53-3.50 (m, 4H), 2.78-2.69 (m, 1H), 2.58-2.55 (m, 1H), 2.40- 2.32 (m, 1H), 2.14-2.07 (m, 1H). MS: (ESI) m/z calculated for C₂₅H₂₃FN₆O₄ [M + H]⁺ 491.1, found 491.2. |
| 392 | 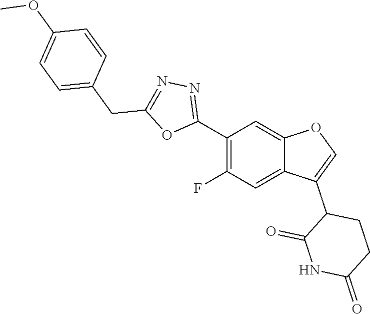 [SMILES: COc1ccc(Cc2nnc(-c3cc4occ(C5CCC(=O)NC5=O)c4cc3F)o2)cc1] | ¹H NMR (400 MHz, d₆-DMSO) δ 8.20-8.14 (m, 2H), 7.74 (d, J = 10.8 Hz, 1H), 7.31 (d, J = 8.8 Hz, 2H), 6.95-6.91 (m, 2H), 4.30 (s, 2H), 4.18 (dd, J = 4.8, 12.4 Hz, 1H), 3.73 (s, 3H), 2.80-2.68 (m, 1H), 2.64-2.55 (m, 1H), 2.38 (br dd, J = 4.4, 12.8 Hz, 1H), 2.15-2.07 (m, 1H). ¹⁹F NMR (376 MHz, d₆-DMSO) δ −118.79. MS: (ESI) m/z calculated for C₂₃H₁₈FN₃O₅ [M + H]⁺ 436.1, found 436.2. |
| 393 | 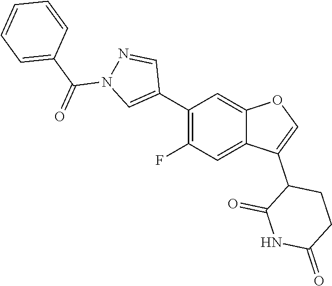 [SMILES: O=C1CCC(c2coc3cc(-c4cnn(C(=O)c5ccccc5)c4)c(F)cc23)C(=O)N1] | ¹H NMR (400 MHz, d₆-DMSO) δ 10.93 (br s, 1H), 8.95 (d, J = 1.6 Hz, 1H), 8.53 (s, 1H), 8.22 (d, J = 6.0 Hz, 1H), 8.09-8.01 (m, 3H), 7.71 (d, J = 7.2 Hz, 1H), 7.65-7.56 (m, 3H), 4.15 (dd, J = 4.8, 12.0 Hz, 1H), 2.82-2.69 (m, 1H), 2.61 (br d, J = 4.0 Hz, 1H), 2.44- 2.35 (m, 1H), 2.18-2.07 (m, 1H). ¹⁹F NMR (376 MHz, d₆-DMSO) δ −120.98. MS: (ESI) m/z [M + H]⁺ 418.4. |
| 394 | 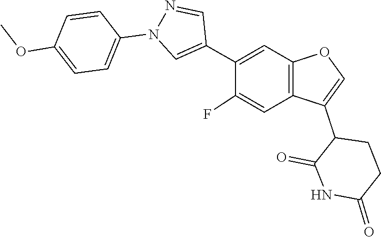 [SMILES: COc1ccc(-n2cc(-c3cc4occ(C5CCC(=O)NC5=O)c4cc3F)cn2)cc1] | ¹H NMR (400 MHz, d₆-DMSO) δ 7.99 (s, 1H), 7.68 (d, J = 6.4 Hz, 1H), 7.53 (br d, J = 10.5 Hz, 3H), 7.13-6.93 (m, 4H), 4.14 (dd, J = 4.8, 12.4 Hz, 1H), 3.81 (s, 3H), 2.79-2.69 (m, 1H), 2.63-2.54 (m, 1H), 2.41-2.32 (m, 1H), 2.17-2.07 (m, 1H). ¹⁹F NMR (376 MHz, d₆-DMSO) δ −124.96. MS: (ESI) m/z [M + H]⁺ 420.3. |
| 395 | 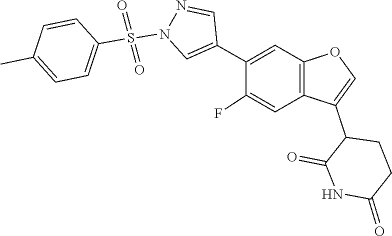 [SMILES: Cc1ccc(S(=O)(=O)n2cc(-c3cc4occ(C5CCC(=O)NC5=O)c4cc3F)cn2)cc1] | ¹H NMR (400 MHz, d₆-DMSO) δ 10.92 (s, 1H), 8.82 (s, 1H), 8.41 (s, 1H), 8.11 (d, J = 8.0 Hz, 1H), 8.01 (s, 1H), 7.94 (d, J = 8.4 Hz, 2H), 7.58 (d, J = 11.2 Hz, 1H), 7.49 (d, J = 8.4 Hz, 2H), 4.13 (dd, J = 4.8, 12.0 Hz, 1H), 2.76- 2.69 (m, 1H), 2.59-2.54 (m, 2H), 2.40 (s, 3H), 2.12-2.07 (m, 1H). MS: (ESI) m/z [M + H]⁺ 468.3. |
| 396 | 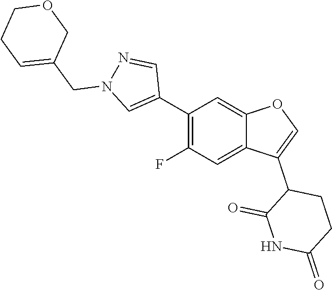 [SMILES: O=C1CCC(c2coc3cc(-c4cnn(CC5=CCCOC5)c4)c(F)cc23)C(=O)N1] | ¹H NMR (400 MHz, d₆-DMSO) δ 10.90 (s, 1H), 8.17 (d, J = 2.4 Hz, 1H), 8.00 (s, 1H), 7.98-7.91 (m, 2H), 7.50 (d, J = 11.2 Hz, 1H), 5.77 (br s, 1H), 4.71 (s, 2H), 4.16-4.06 (m, 1H), 3.92 (br d, J = 1.6 Hz, 2H), 3.62 (t, J = 5.5 Hz, 2H), 2.80-2.67 (m, 1H), 2.62-2.54 (m, 1H), 2.35 (br dd, J = 4.0, 12.4 Hz, 1H), 2.16-2.04 (m, 3H) ¹⁹F NMR (376 MHz, d₆- DMSO) δ −121.73. MS: (ESI) m/z [M + H]⁺ 410.3 |
| 397 | 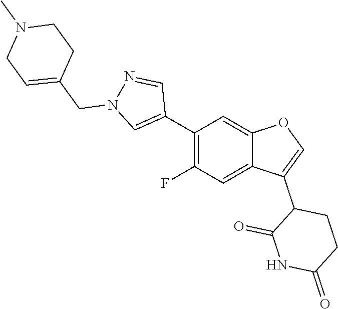 [SMILES: CN1CC=C(Cn2cc(-c3cc4occ(C5CCC(=O)NC5=O)c4cc3F)cn2)CC1] | ¹H NMR (400 MHz, d₆-DMSO) δ 10.90 (s, 1H), 8.15 (s, 1H), 8.00 (s, 1H), 7.96 (s, 1H), 7.94 (s, 1H), 7.49 (d, J = 12.0 Hz, 1H), 5.54 (br s, 1H), 4.73 (s, 2H), 4.11 (dd, J = 4.4, 12.0 Hz, 1H), 3.01 (br s, 3H), 2.75-2.71 (m, 1H), 2.71-2.66 (m, 1H), 2.58 (br t, J = 8.0 Hz, 2H), 2.41-2.35 (m, 1H), 2.32 (s, 3H), 2.17- 2.06 (m, 1H), 2.01 (br s, 1H). ¹⁹F NMR (376 MHz, d₆-DMSO) δ −121.72. MS: (ESI) m/z [M + H]⁺ 423.4. |
| 398 | 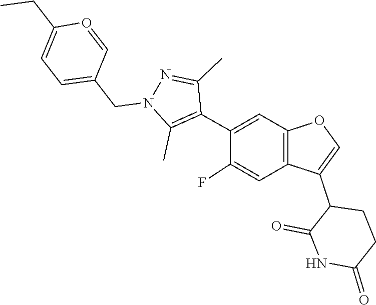 | ¹H NMR (400 MHz, d₆-DMSO) δ 10.96- 10.89 (m, 1H), 7.98 (s, 1H), 7.54-7.48 (m, 2H), 7.18 (d, J = 8.8 Hz, 2H), 6.95-6.89 (m, 2H), 5.20 (s, 2H), 4.14 (dd, J = 4.8, 12.4 Hz, 1H), 3.72-3.71 (m, 1H), 3.73 (s, 2H), 2.85- 2.64 (m, 1H), 2.62-2.52 (m, 2H), 2.44-2.28 (m, 1H), 2.13 (s, 3H), 2.09-2.06 (m, 3H). ¹⁹F NMR (376 MHz, d₆-DMSO) δ −121.43. MS: (ESI) m/z [M + H]⁺ 462.4 |
| 399 | 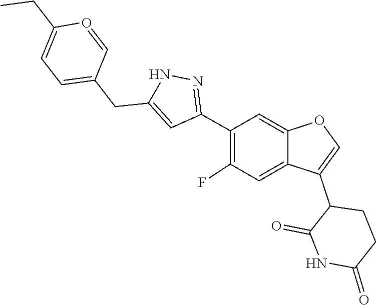 | ¹H NMR (400 MHz, d₆-DMSO) δ 10.90 (s, 1H), 8.05-7.90 (m, 2H), 7.51 (d, J = 10.8 Hz, 1H), 7.21 (d, J = 8.4 Hz, 2H), 6.88 (d, J = 8.8 Hz, 2H), 6.40 (d, J = 4.4 Hz, 1H), 4.13 (dd, J = 4.8, 12.0 Hz, 1H), 3.92 (s, 2H), 3.72 (s, 3H), 2.80-2.65 (m, 1H), 2.59 (br d, J = 3.6 Hz, 1H), 2.40-2.29 (m, 1H), 2.10 (qd, J = 4.8, 12.0 Hz, 1H). ¹⁹F NMR (376 MHz, d₆-DMSO) δ −77.7. MS: (ESI) m/z calculated for C₂₄H₂₀FN₃O₄ [M + H]⁺ 434.1, found 434.3. |
| 400 | 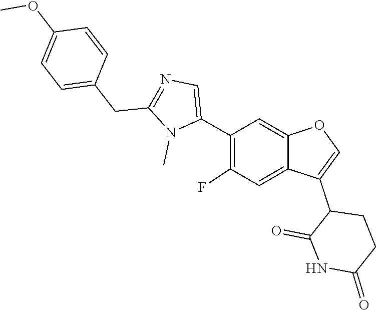 [SMILES: COc1ccc(Cc2ncc(-c3cc4occ(C5CCC(=O)NC5=O)c4cc3F)n2C)cc1] | ¹H NMR (400 MHz, d₆-DMSO) δ 10.92 (s, 1H), 8.04 (s, 1H), 7.65 (d, J = 4.0 Hz, 1H), 7.59 (d, J = 8.0 Hz, 1H), 7.18 (d, J = 8.0 Hz, 2H), 6.97 (br s, 1H), 6.89 (d, J = 8.0 Hz, 2H), 4.15 (dd, J = 4.0, 12.0 Hz, 1H), 4.07 (s, 2H), 3.72 (s, 3H), 3.38 (s, 3H), 2.79-2.69 (m, 1H), 2.62- 2.54 (m, 1H), 2.43-2.33 (m, 1H), 2.14-2.07 (m, 1H). ¹⁹F NMR (376 MHz, d₆-DMSO) δ −121.2. MS: (ESI) m/z [M + H]⁺ 448.2. |
| 401 | 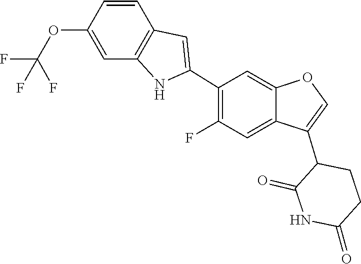 [SMILES: O=C1CCC(c2coc3cc(-c4cc5ccc(OC(F)(F)F)cc5[nH]4)c(F)cc23)C(=O)N1] | ¹H NMR (400 MHz, DMSO-d₆) δ 11.79 (s, 1H), 10.93 (s, 1H), 8.11 (d, J = 6.0 Hz, 1H), 8.05 (s, 1H), 7.60-7.71 (m, 2H), 7.38 (s, 1H), 6.98-7.05 (m, 2H), 4.16 (dd, J = 12.4, 4.8 Hz, 1H), 2.70-2.80 (m, 1H), 2.60-2.63 (m, 1H), 2.36-2.41 (m, 1H), 2.09-2.17 (m, 1H). LCMS: calc. for C₂₂H₁₄F₄N₂O₄: 446.4, found: [M + H]⁺ 447.1. |
| 402 | 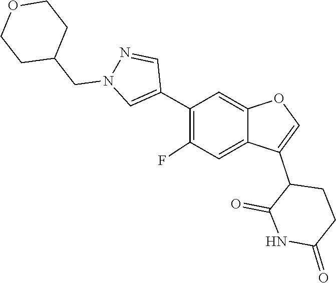 [SMILES: O=C1CCC(c2coc3cc(-c4cnn(CC5CCOCC5)c4)c(F)cc23)C(=O)N1] | ¹H NMR (400 MHz, DMSO-d₆) δ 11.27- 10.42 (m, 1H), 8.18 (d, J = 2.4 Hz, 1H), 7.99 (s, 1H), 7.96-7.90 (m, 2H), 7.50 (d, J = 11.2 Hz, 1H), 4.12 (dd, J = 12.0, 4.8 Hz, 1H), 4.06 (d, J = 7.2 Hz, 2H), 3.83 (br dd, J = 11.2, 2.8 Hz, 2H), 3.30-3.22 (m, 2H), 2.80-2.67 (m, 1H), 2.58 (td, J = 17.2,3.6 Hz, 1H), 2.42- 2.30 (m, 1H), 2.17-2.01 (m, 2H), 1.42 (br d, J = 11.2 Hz, 2H), 1.31-1.21 (m, 2H). ¹⁹F NMR (400 MHz, DMSO-d₆) δ −121.73. MS: (ESI) m/z calculated for C₂₂H₂₂FN₃O₄ [M + H]⁺ 412.1, found 412.3. |
| 403 | 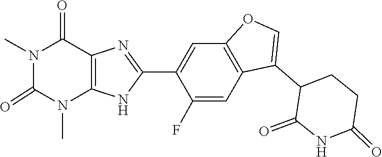 [SMILES: Cn1c(=O)c2nc(-c3cc4occ(C5CCC(=O)NC5=O)c4cc3F)[nH]c2n(C)c1=O] | ¹H NMR (400 MHz, d₆-DMSO) δ 10.93 (s, 1H), 8.15-8.06 (m, 2H), 7.63 (d, J = 10.8 Hz, 1H), 4.17 (dd, J = 4.8, 12.4 Hz, 1H), 3.51 (s, 3H), 3.27 (s, 3H), 2.80-2.69 (m, 1H), 2.60 (td, m, 1H), 2.39 (br dd, J = 4.4, 12.0 Hz, 1H), 2.17-2.08 (m, 1H). ¹⁹F NMR (376 MHz, d₆- DMSO) δ −120.53. MS: (ESI) m/z [M + H]⁺ 426.1. |
| 404 | 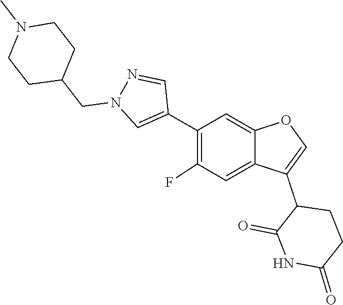 [SMILES: CN1CCC(Cn2cc(-c3cc4occ(C5CCC(=O)NC5=O)c4cc3F)cn2)CC1] | ¹H NMR (400 MHz, DMSO-d₆) δ 10.90 (s, 1H), 8.18 (d, J = 2.4 Hz, 1H), 7.98 (s, 1H), 7.96-7.90 (m, 2H), 7.50 (d, J = 11.2 Hz, 1H), 4.14-4.09 (m, 1H), 4.04 (br d, J = 7.2 Hz, 2H), 2.76 (br d, J = 11.2 Hz, 2H), 2.71-2.65 (m, 1H), 2.62-2.59 (m, 1H), 2.35 (br dd, J = 12.6, 4.0 Hz, 1H), 2.15 (s, 3H), 2.12-1.98 (m, 1H), 1.89-1.77 (m, 3H), 1.48 (br d, J = 12.4 Hz, 2H), 1.29-1.20 (m, 2H). ¹⁹F NMR (400 MHz, DMSO-d₆) δ −121.74. MS: (ESI) m/z [M + H]⁺ 425.4. |
| 405 | 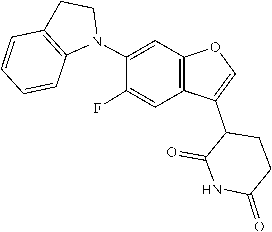 [SMILES: O=C1CCC(c2coc3cc(N4CCc5ccccc54)c(F)cc23)C(=O)N1] | ¹H NMR (400 MHz, DMSO-d₆) δ 10.92 (s, 1H), 7.93 (s, 1H), 7.64 (d, J = 6.4 Hz, 1H), 7.56 (d, J = 11.2 Hz, 1H), 7.18 (d, J = 7.2 Hz, 1H), 7.00 (t, J = 7.6 Hz, 1H), 6.71 (t, J = 7.2 Hz, 1H), 6.45 (dd, J = 7.6, 3.6 Hz, 1H), 4.12 (dd, J = 12.4,4.8 Hz, 1H), 3.92 (t, J = 8.4 Hz, 2H), 3.11 (br t, J = 8.4 Hz, 2H), 2.79-2.69 (m, 1H), 2.63-2.55 (m, 1H), 2.44-2.32 (m, 1H), 2.18-2.06 (m, 1H). ¹⁹F NMR (400 MHz, DMSO-d₆) δ −125.17. MS: (ESI) m/z [M + H]⁺ 365.3 |
| 406 | 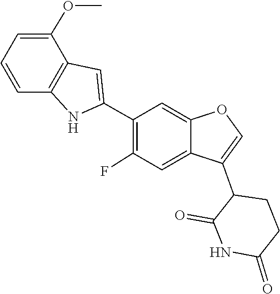 [SMILES: COc1cccc2[nH]c(-c3cc4occ(C5CCC(=O)NC5=O)c4cc3F)cc12] | ¹H NMR (400 MHz, DMSO-d₆) δ 11.52 (s, 1H), 10.93 (s, 1H), 8.10 (d, J = 6.0 Hz, 1H), 8.02 (s, 1H), 7.59 (d, J = 11.6 Hz, 1H), 7.01- 7.10 (m, 2H), 6.88-6.99 (m, 1H), 6.48-6.57 (m, 1H), 4.15 (dd, J = 12.4, 4.8 Hz, 1H), 3.89 (s, 3H), 2.69-2.81 (m, 1H), 2.57-2.63 (m, 1H), 2.35-2.43 (m, 1H), 2.09-2.22 (m, 1H). LCMS: calc. for C₂₂H₁₇FN₂O₄: 392.1, found: [M + H]⁺ 393.1. |
| 407 | 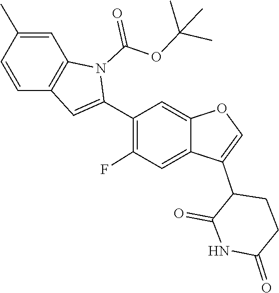 [SMILES: Cc1ccc2cc(-c3cc4occ(C5CCC(=O)NC5=O)c4cc3F)n(C(=O)OC(C)(C)C)c2c1] | ¹H NMR (400 MHz, DMSO-d₆) δ 10.91 (s, 1H), 8.03 (s, 1H), 7.96 (s, 1H), 7.76 (d, J = 5.6 Hz, 1H), 7.50-7.56 (m, 2H), 7.09-7.13 (m, 1H), 6.76 (s, 1H), 4.17 (dd, J = 12.4, 4.8 Hz, 1H), 2.70-2.80 (m, 1H), 2.55-2.63 (m, 1H), 2.46 (s, 3H), 2.31-2.41 (m, 1H), 2.08-2.16 (m, 1H), 1.25 (s, 9H). LCMS: calc. for C₂₇H₂₅FN₂O₅: 476.2, found: [M − Boc + H]⁺ 377.1. |
| 408 | 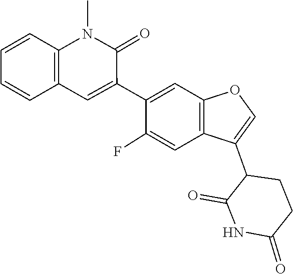 [SMILES: Cn1c(=O)c(-c2cc3occ(C4CCC(=O)NC4=O)c3cc2F)cc2ccccc21] | ¹H NMR (400 MHz, DMSO-d₆) δ 10.91 (br s, 1H), 8.08 (s, 1H), 8.02 (s, 1H), 7.79 (dd, J = 1.2, 7.6 Hz, 1H), 7.73-7.69 (m, 1H), 7.69- 7.65 (m, 1H), 7.61-7.57 (m, 1H), 7.52 (d, J = 10.0 Hz, 1H), 7.32 (t, J = 7.6 Hz, 1H), 4.17 (dd, J = 4.8, 12.4 Hz, 1H), 3.71 (s, 3H), 2.82- 2.69 (m, 1H), 2.64-2.56 (m, 1H), 2.46-2.34 (m, 1H), 2.17-2.09 (m, 1H). MS: (ESI) m/z [M + H]⁺ 405.2. |
| 409 | 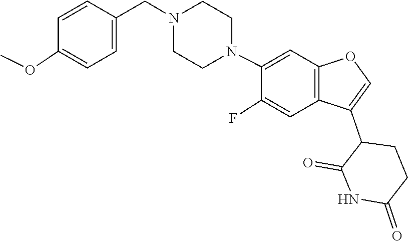 [SMILES: COc1ccc(CN2CCN(c3cc4occ(C5CCC(=O)NC5=O)c4cc3F)CC2)cc1] | ¹H NMR (400 MHz, DMSO-d₆) 10.87 (s, 1H), 8.13 (s, 1H), 7.82 (s, 1H), 7.37 (d, J = 12.4 Hz, 1H), 7.30-7.14 (m, 3H), 6.90 (d, J = 8.4 Hz, 2H), 4.12-4.01 (m, 1H), 3.74 (s, 3H), 3.49 (br s, 2H), 3.01 (br s, 4H), 2.75-2.65 (m, 1H), 2.61-2.51 (m, 5H), 2.36-2.24 (m, 1H), 2.07 (dt, J = 8.8, 4.4 Hz, 1H). ¹⁹F NMR (400 MHz, DMSO-d₆) δ −128.65. MS: (ESI) m/z [M + H]⁺ 452.4. |
| 410 | 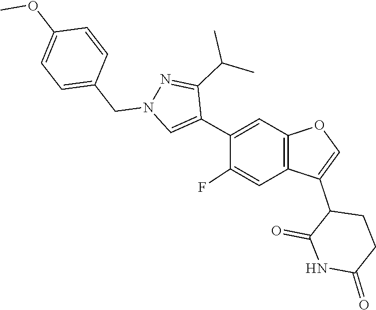 [SMILES: COc1ccc(Cn2cc(-c3cc4occ(C5CCC(=O)NC5=O)c4cc3F)c(C(C)C)n2)cc1] | ¹H NMR (400 MHz, DMSO-d₆) δ 10.91 (s, 1H), 7.96 (s, 1H), 7.80 (d, J = 1.2 Hz, 1H), 7.53-7.47 (m, 2H), 7.27 (d, J = 8.8 Hz, 2H), 6.97-6.89 (m, 2H), 5.23 (s, 2H), 4.13 (dd, J = 4.8, 12.0 Hz, 1H), 3.73 (s, 3H), 3.01 (td, J = 6.8, 13.2 Hz, 1H), 2.79-2.68 (m, 1H), 2.61- 2.55 (m, 1H), 2.44-2.31 (m, 1H), 2.15-2.04 (m, 1H), 1.14 (d, J = 6.8 Hz, 6H). MS: (ESI) m/z [M + H]⁺ 476.2. |
| 411 | 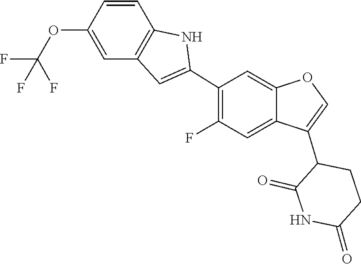 [SMILES: O=C1CCC(c2coc3cc(-c4cc5cc(OC(F)(F)F)ccc5[nH]4)c(F)cc23)C(=O)N1] | LCMS: calc. for C₂₂H₁₄F₄N₂O₅: 446.1, found: [M + H]⁺ 447.1. |
| 412 | 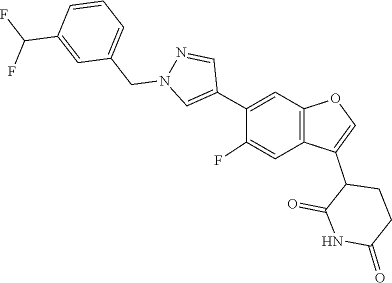 [SMILES: O=C1CCC(c2coc3cc(-c4cnn(Cc5cccc(C(F)F)c5)c4)c(F)cc23)C(=O)N1] | ¹H NMR (400 MHz, d₆-DMSO) δ 8.36 (d, J = 2.4 Hz, 1H), 8.04 (s, 1H), 7.98-7.92 (m, 2H), 7.56-7.43 (m, 5H), 7.03 (s, 1H), 5.46 (s, 2H), 4.11 (dd, J = 4.8, 12.0 Hz, 1H), 2.77-2.59 (m, 2H), 2.42-2.28 (m, 2H), 2.15-2.06 (m, 1H). ¹⁹F NMR (376 MHz, d₆-DMSO) δ −121.7, −109.7. MS: (ESI) m/z [M + H]⁺ 454.3. |
| 413 | 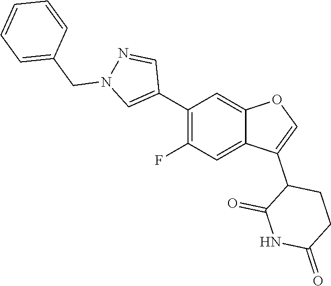 [SMILES: O=C1CCC(c2coc3cc(-c4cnn(Cc5ccccc5)c4)c(F)cc23)C(=O)N1] | ¹H NMR (400 MHz, CD₃OD) δ 8.17 (d, J = 2.0 Hz, 1H), 8.00 (s, 1H), 7.82-7.77 (m, 2H), 7.40 (s, 1H), 7.38 (d, J = 2.8 Hz, 1H), 7.36 (s, 1H), 7.34 (d, J = 1.2 Hz, 1H), 7.32 (d, J = 1.6 Hz, 1H), 7.30 (s, 1H), 5.42 (s, 2H), 4.13 (dd, J = 4.8 , 11.6 Hz, 1H), 2.88-2.75 (m, 2H), 2.46-2.35 (m, 1H), 2.34-2.27 (m, 1H). ¹⁹F NMR (376 MHz, CD₃OD) δ −123.4. MS: (ESI) m/z [M + H]⁺ 404.3. |
| 414 | 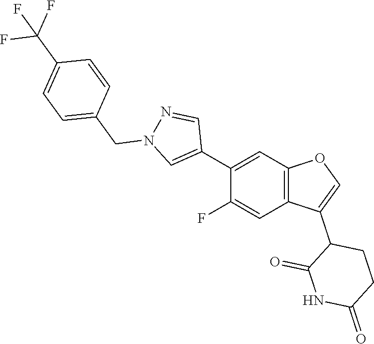 [SMILES: O=C1CCC(c2coc3cc(-c4cnn(Cc5ccc(C(F)(F)F)cc5)c4)c(F)cc23)C(=O)N1] | ¹H NMR (400 MHz, d6-DMSO) δ 11.05- 10.82 (m, 1H), 8.38 (d, J = 2.8 Hz, 1H), 8.07 (s, 1H), 8.01-7.92 (m, 2H), 7.74 (d, J = 8.0 Hz, 2H), 7.55-7.45 (m, 3H), 5.52 (s, 2H), 4.12 (dd, J = 4.8, 12.0 Hz, 1H), 2.76 -2.68 (m, 1H), 2.59 (m, 1H), 2.37 (m, 1H), 2.14- 2.07 (m, 1H). ¹⁹F NMR (376 MHz, d₆-DMSO) δ −60.9, −121-7. MS: (ESI) m/z [M + H]⁺ 472.2. |
| 415 | 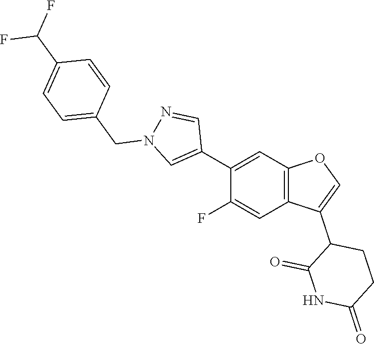 [SMILES: O=C1CCC(c2coc3cc(-c4cnn(Cc5ccc(C(F)F)cc5)c4)c(F)cc23)C(=O)N1] | ¹H NMR (400 MHz, d₆-DMSO) δ 11.03- 10.80 (m, 1H), 8.35 (d, J = 2.4 Hz, 1H), 8.05 (s, 1H), 7.96 (d, J = 8.0 Hz, 1H), 7.94 (s, 1H), 7.57 (s, 1H), 7.55 (s, 1H), 7.54-7.47 (m, 1H), 7.42 (s, 1H), 7.40 (s, 1H), 7.01 (s, 1H), 5.46 (s, 2H), 4.12 (dd, J = 4.8, 12.0 Hz, 1H), 2.75- 2.68 (m, 1H), 2.63-2.54 (m, 1H), 2.35 (br dd, J = 4.0, 12.4 Hz, 1H), 2.14-2.07 (m, 1H). ¹⁹F NMR (376 MHz, d₆-DMSO) δ −109.36, −121.69. MS: (ESI) m/z [M + H]⁺ 454.3. |
| 416 | 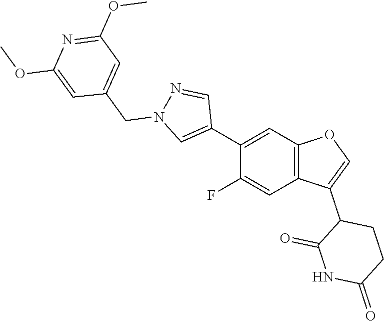 [SMILES: COc1cc(Cn2cc(-c3cc4occ(C5CCC(=O)NC5=O)c4cc3F)cn2)cc(OC)n1] | ¹H NMR (400 MHz, DMSO-d₆) δ 10.91 (s, 1H), 8.33 (d, J = 2.4 Hz, 1H), 8.06 (s, 1H), 7.99-7.94 (m, 2H), 7.51 (d, J = 11.3 Hz, 1H), 6.17 (s, 2H), 5.35 (s, 2H), 4.12 (dd, J = 4.8, 12.0 Hz, 1H), 3.82 (s, 6H), 2.78-2.66 (m, 1H), 2.62-2.54 (m, 1H), 2.39-2.31 (m, 1H), 2.16-2.06 (m, 1H) ¹⁹F NMR (376 MHz, d₆- DMSO) δ −121.70. MS: (ESI) m/z [M + H]⁺ 465.2. |
| 417 | 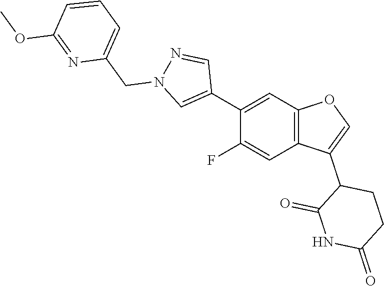 [SMILES: COc1cccc(Cn2cc(-c3cc4occ(C5CCC(=O)NC5=O)c4cc3F)cn2)n1] | ¹H NMR (400 MHz, CD₃OD) δ 8.32 (d, J = 2.0 Hz, 1H), 8.11-8.06 (m, 1H), 7.88-7.77 (m, 3H), 7.40 (d, J = 11.2 Hz, 1H), 6.98-6.89 (m, 1H), 6.85 (d, J = 7.2 Hz, 1H), 5.51 (s, 2H), 4.12 (dd, J = 5.2, 11.6 Hz, 1H), 3.98 (s, 3H), 2.87-2.69 (m, 2H), 2.46-2.24 (m, 2H). ¹⁹FNMR (376 MHz, CD₃OD) δ −123.41. MS: (ESI) m/z [M + H]⁺ 435.3. |
| 418 | 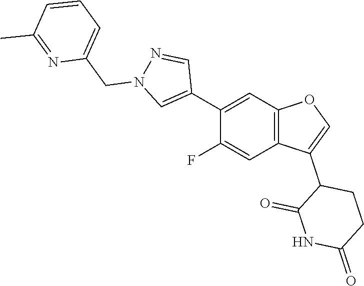 [SMILES: Cc1cccc(Cn2cc(-c3cc4occ(C5CCC(=O)NC5=O)c4cc3F)cn2)n1] | ¹H NMR (400 MHz, d₆-DMSO) δ 10.91 (s, 1H), 8.34 (d, J = 2.4 Hz, 1H), 8.05 (s, 1H), 8.00-7.92 (m, 2H), 7.66 (t, J = 7.7 Hz, 1H), 7.51 (d, J = 11.2 Hz, 1H), 7.18 (d, J = 8.0 Hz, 1H), 6.86 (d, J = 7.6 Hz, 1H), 5.44 (s, 2H), 4.19-4.05 (m, 1H), 2.80-2.66 (m, 1H), 2.62- 2.54 (m, 1H), 2.46 (s, 3H), 2.36 (br dd, J = 4.4, 12.8 Hz, 1H), 2.15-2.04 (m, 1H). ¹⁹FNMR (376 MHz, d₆-DMSO) δ −121.67. MS: (ESI) m/z [M + H]⁺ 419.3. |
| 419 | 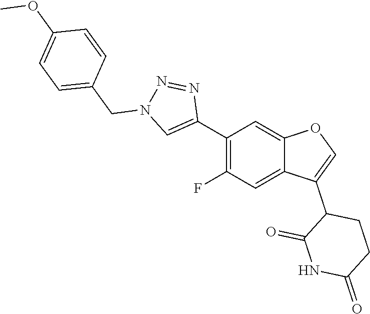 [SMILES: COc1ccc(Cn2cc(-c3cc4occ(C5CCC(=O)NC5=O)c4cc3F)nn2)cc1] | ¹H NMR (400 MHz, DMSO-d₆) δ 10.91 (s, 1H), 8.49 (d, J = 4.0 Hz, 1H), 8.20 (d, J = 1.6 Hz, 1H), 7.99 (s, 1H), 7.60 (d, J = 10.8 Hz, 1H), 7.37 (d, J = 8.8 Hz, 2H), 6.96-6.93 (m, 2H), 5.59 (s, 2H), 4.16-4.12 (m, 1H), 3.73 (s, 3H), 2.73-2.60 (m, 1H), 2.59-2.56 (m, 1H), 2.39-2.38 (m, 1H), 2.13-2.09 (m, 1H). ¹⁹F NMR (376 MHz, DMSO-d₆) δ −121.63. MS: (ESI) m/z [M + H]⁺ 435.1. |
| 420 | 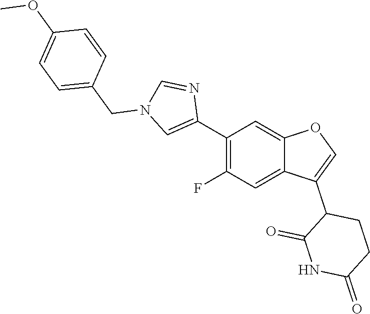 [SMILES: COc1ccc(Cn2cnc(-c3cc4occ(C5CCC(=O)NC5=O)c4cc3F)c2)cc1] | ¹H NMR (400 MHz, DMSO-d₆) δ 8.12-8.07 (m, 1H), 7.96-7.87 (m, 2H), 7.58-7.53 (m, 1H), 7.47 (d, J = 11.2 Hz, 1H), 7.32 (d, J = 8.8 Hz, 2H), 6.93 (d, J = 8.6 Hz, 2H), 5.18 (s, 2H), 4.18-4.05 (m, 1H), 3.73 (s, 3H), 2.71- 2.66 (m, 1H), 2.61-2.58 (m, 1H), 2.38-2.33 (m, 1H), 2.14-2.08 (m, 1H). MS: (ESI) m/z [M + H]⁺ 434.2. |
| 421 | 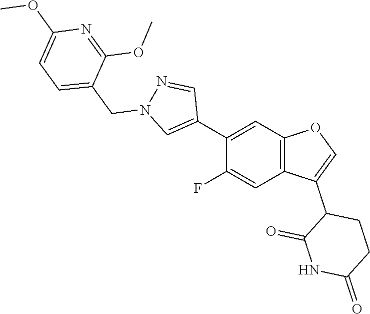 [SMILES: COc1ccc(Cn2cc(-c3cc4occ(C5CCC(=O)NC5=O)c4cc3F)cn2)c(OC)n1] | ¹H NMR (400 MHz, d₆-DMSO) δ 11.01- 10.82 (m, 1H), 8.18 (d, J = 2.0 Hz, 1H), 7.98 (s, 1H), 7.96-7.92 (m, 2H), 7.52-7.45 (m, 2H), 6.38 (d, J = 8.0 Hz, 1H), 5.24 (s, 2H), 4.11 (dd, J = 4.8, 12.4 Hz, 1H), 3.92 (s, 3H), 3.85 (s, 3H), 2.78-2.67 (m, 1H), 2.62-2.53 (m, 1H), 2.41-2.29 (m, 1H), 2.15-2.06 (m, 1H). ¹F NMR (376 MHz, d₆-DMSO) δ −121.8. MS: (ESI) m/z calculated for C₂₄H₂₁FN₄O₅ [M + H]⁺ 465.1, found 435.4. |
| 422 | 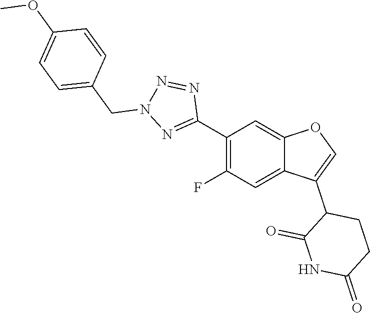 [SMILES: COc1ccc(Cn2nnc(-c3cc4occ(C5CCC(=O)NC5=O)c4cc3F)n2)cc1] | ¹H NMR (400 MHz, DMSO-d₆) δ 10.93 (s, 1H), 8.19 (d, J = 5.6 Hz, 1H), 8.13 (s, 1H), 7.70 (d, J = 10.4 Hz, 1H), 7.41 (d, J = 8.4 Hz, 2H), 7.00-6.93 (m, 2H), 5.95 (s, 2H), 4.18 (dd, J = 12.4, 4.8 Hz, 1H), 3.75 (s, 3H), 2.79- 2.69 (m, 1H), 2.39 (br dd, J = 12.4, 4.0 Hz, 1H), 2.16-2.08 (m, 1H), 2.05-1.91 (m, 1H). ¹⁹F NMR (376 MHz, DMSO-d₆) δ −119.67. MS: (ESI) m/z [M + H]⁺ 436.3. |
| 423 | 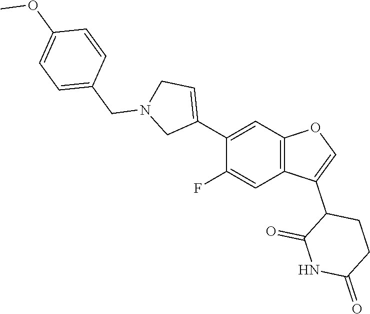 [SMILES: COc1ccc(CN2CC=C(c3cc4occ(C5CCC(=O)NC5=O)c4cc3F)C2)cc1] | ¹H NMR (400 MHz, MeOD) δ 8.42 (br s, 1H), 7.84 (s, 1H), 7.55 (d, J = 5.6 Hz, 1H), 7.44 (d, J = 8.4 Hz, 2H), 7.40 (d, J = 11.6 Hz, 1H), 7.01 (d, J = 8.4 Hz, 2H), 6.45 (s, 1H), 4.38 (s, 2H), 4.32 (s, 2H), 4.13 (d, J = 4.8, 1H), 4.11 (s, 2H), 3.83 (s, 3H), 2.83-2.67 (m, 2H), 2.44- 2.34 (m, 1H), 2.31-2.20 (m, 1H). ¹⁹F NMR (400 MHz, MeOD) δ 121.29. MS: (ESI) m/z [M + H]⁺ 435.3. |
| 424 | 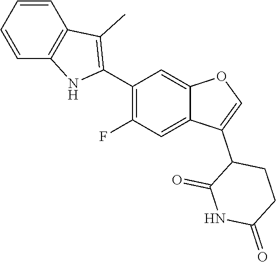 [SMILES: Cc1c(-c2cc3occ(C4CCC(=O)NC4=O)c3cc2F)[nH]c2ccccc12] | ¹H NMR (400 MHz, DMSO-d₆) δ 11.16 (s, 1H), 10.96 (br s, 1H), 8.05 (s, 1H), 7.74 (d, J = 5.8 Hz, 1H), 7.62 (d, J = 10.4 Hz, 1H), 7.54 (d, J = 7.9 Hz, 1H), 7.37 (d, J = 8.1 Hz, 1H), 7.13 (t, J = 7.1 Hz, 1H), 7.06-7.00 (m, 1H), 4.18 (dd, J = 4.8, 12.4 Hz, 1H), 2.81-2.71 (m, 1H), 2.64-2.57 (m, 1H), 2.47-2.39 (m, 1H), 2.27 (s, 3H), 2.17-2.09 (m, 1H). ¹⁹F NMR (376 MHz, d₆-DMSO) δ −121.0. MS: (ESI) m/z [M + H]⁺ 377.4. |
| 425 | 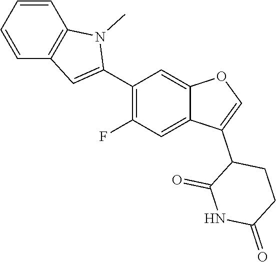 [SMILES: Cn1c(-c2cc3occ(C4CCC(=O)NC4=O)c3cc2F)cc2ccccc21] | ¹H NMR (400 MHz, DMSO-d₆) δ 11.08- 10.87 (m, 1H), 8.09 (s, 1H), 7.78 (d, J = 5.9 Hz, 1H), 7.66 (d, J = 10.1 Hz, 1H), 7.60 (d, J = 7.8 Hz, 1H), 7.52 (d, J = 8.1 Hz, 1H), 7.26- 7.20 (m, 1H), 7.13-7.07 (m, 1H), 6.61 (s, 1H), 4.23-4.16 (m, 1H), 3.66 (s, 3H), 2.78- 2.71 (m, 1H), 2.64-2.57 (m, 1H), 2.45-2.39 (m, 1H), 2.19-2.10 (m, 1H). ¹⁹F NMR (376 MHz, d₆-DMSO) δ −121.3. MS: (ESI) m/z [M + H]⁺ 377.3 |
| 426 | 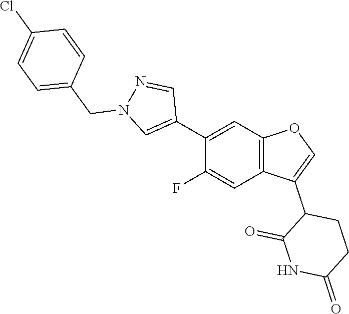 [SMILES: O=C1CCC(c2coc3cc(-c4cnn(Cc5ccc(Cl)cc5)c4)c(F)cc23)C(=O)N1] | ¹H NMR (400 MHz, d₆-DMSO) δ 10.90 (s, 1H), 8.33 (d, J = 2.8 Hz, 1H), 8.04 (s, 1H), 7.98-7.93 (m, 2H), 7.51 (d, J = 11.2 Hz, 1H), 7.43 (d, J = 8.8 Hz, 2H), 7.32 (d, J = 8.8 Hz, 2H), 5.42-5.38 (m, 1H), 5.40 (s, 1H), 4.12 (dd, J = 5.2, 12.4 Hz, 1H), 2.79-2.71 (m, 1H), 2.63-2.59 (m, 1H), 2.37 (br d, J = 3.6 Hz, 1H), 2.16-2.07 (m, 1H). ¹⁹F NMR (376 MHz, d₆-DMSO) δ −121.7. MS: (ESI) m/z [M + H]⁺ 438.3. |
| 427 | 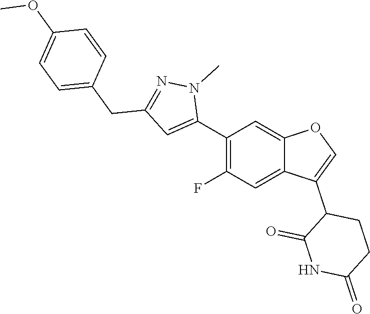 [SMILES: COc1ccc(Cc2cc(-c3cc4occ(C5CCC(=O)NC5=O)c4cc3F)n(C)n2)cc1] | ¹H NMR (400 MHz, d₆-DMSO) δ 10.92 (s, 1H), 8.06 (s, 1H), 7.71 (d, J = 5.6 Hz, 1H), 7.61 (d, J = 10.0 Hz, 1H), 7.20 (d, J = 8.4 Hz, 2H), 6.90-6.81 (m, 2H), 6.16 (s, 1H), 4.16 (dd, J = 4.8, 12.4 Hz, 1H), 3.83 (s, 2H), 3.71 (s, 3H), 3.69 (s, 3H), 2.79-2.68 (m, 1H), 2.62- 2.58 (m, 1H), 2.44-2.30 (m, 1H), 2.10 (dt, J = 4.0, 8.8 Hz, 1H). ¹⁹F NMR (376 MHz, d₆- DMSO) δ −121.3. MS: (ESI) m/z [M + H]⁺ 448.4. |

### Example 97:3-[6-[1-[[4-(cyclopropylmethoxy)phenyl]methyl]pyrazol-4-yl]-1,2-benzoxazol-3-yl]piperidine-2,6-dione

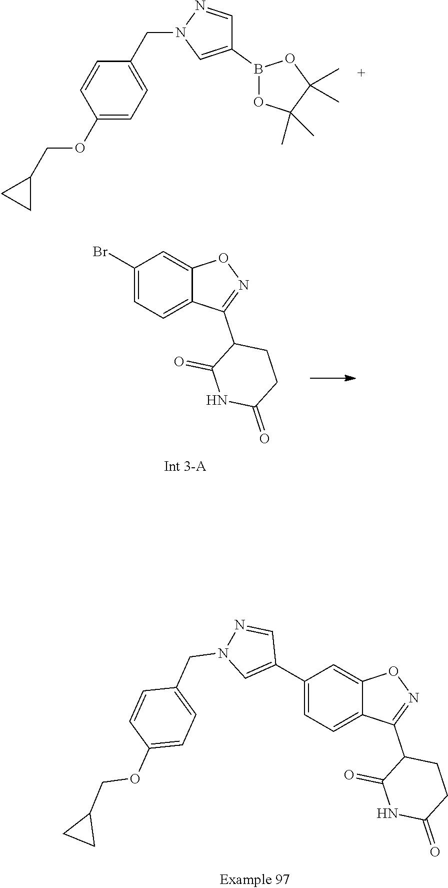 [SMILES: CC1(C)OB(c2cnn(Cc3ccc(OCC4CC4)cc3)c2)OC1(C)C ; O=C1CCC(c2noc3cc(Br)ccc23)C(=O)N1 ; O=C1CCC(c2noc3cc(-c4cnn(Cc5ccc(OCC6CC6)cc5)c4)ccc23)C(=O)N1]

A mixture of Int 3-A (30 mg, 97.05 μmol, 1 eq), 1-[[4-(cyclopropylmethoxy)phenyl]methyl]-4-(4,4,5,5-tetramethyl-1,3,2-dioxaborolan-2-yl)pyrazole (41.26 mg, 116.46 μmol, 1.2 eq), Pd(dppf) C₁₂ (3.55 mg, 4.85 μmol, 0.05 eq) and KF (16.92 mg, 291.15 μmol, 3 eq) in dioxane (2 mL) and H₂O (0.2 mL) was degassed and purged with N₂ for 3 times, and then the mixture was stirred at 85° C. for 16 hours under N₂ atmosphere. The reaction mixture was filtered directly, and the cake was washed with DCM (10 mL). The filtrate was concentrated under reduced pressure to afford a residue. The residue was purified by prep-HPLC to afford Example 97 (3.7 mg, 7.89 μmol, 8.1% yield) as a white solid. LCMS: calc. for C₂₆H₂₄N₄O₄:456.2, found: [M+H]⁺457.1. ¹H NMR (400 MHZ, DMSO-d₆) δ 11.09 (s, 1H), 8.43 (s, 1H), 8.08 (s, 1H), 7.95 (s, 1H), 7.80 (d, J=8.4 Hz, 1H), 7.62 (d, J=8.4 Hz, 1H), 7.25 (d, J=8.8 Hz, 2H), 6.90 (d, J=8.4 Hz, 2H), 5.27 (s, 2H), 4.52-4.62 (m, 1H), 3.78 (d, J=6.8 Hz, 2H), 2.72-2.82 (m, 1H), 2.52-2.61 (m, 1H), 2.45-2.49 (m, 1H), 2.16-2.26 (m, 1H), 1.15-1.25 (m, 1H), 0.52-0.58 (m, 2H), 0.26-0.32 (m, 2H).

### Example 98:3-[6-[1-[(4-isopropylphenyl)methyl]pyrazol-4-yl]-1,2-benzoxazol-3-yl]piperidine-2,6-dione

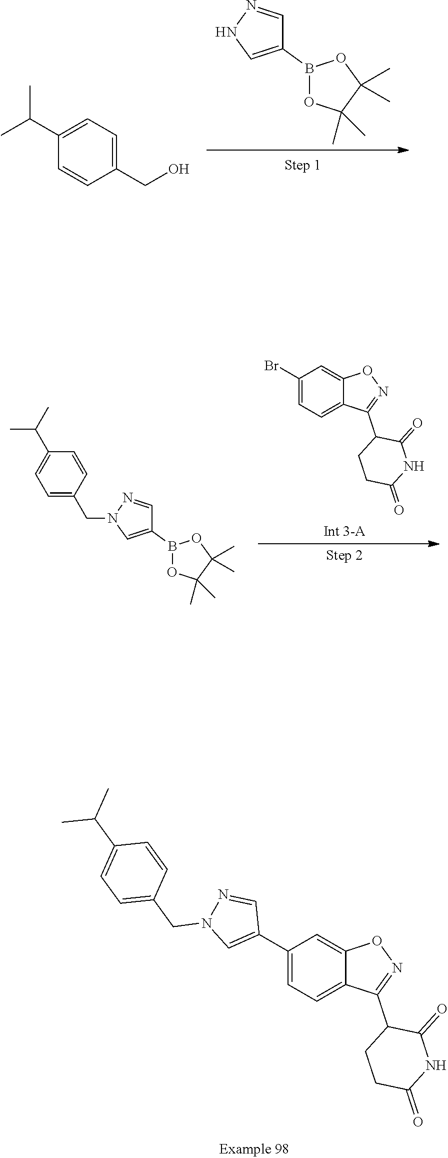 [SMILES: CC(C)c1ccc(CO)cc1 ; CC1(C)OB(c2cn[nH]c2)OC1(C)C ; CC(C)c1ccc(Cn2cc(B3OC(C)(C)C(C)(C)O3)cn2)cc1 ; O=C1CCC(c2noc3cc(Br)ccc23)C(=O)N1 ; CC(C)c1ccc(Cn2cc(-c3ccc4c(C5CCC(=O)NC5=O)noc4c3)cn2)cc1]

Step 1: To a solution of (4-isopropylphenyl) methanol (2.00 g, 13.31 mmol, 2.04 mL, 1 eq), 4-(4,4,5,5-tetramethyl-1,3,2-dioxaborolan-2-yl)-1H-pyrazole (2.58 g, 13.31 mmol, 1 eq) and PPh3 (3.49 g, 13.31 mmol, 1 eq) in THF (20 mL) was added DIAD (2.69 g, 13.31 mmol, 2.59 mL, 1 eq) at 0° C. Then mixture was stirred at 20° C. for 16 h. The reaction mixture was diluted with H₂O (30 mL), adjusted to pH=4 with HCl (1 N) and then it was extracted with EtOAc (50 mL×3). The combined organic layer was concentrated under reduced pressure to afford a residue. The residue was purified by column chromatography to afford 1-[(4-isopropylphenyl)methyl]-4-(4,4,5,5-tetramethyl-1,3,2-dioxaborolan-2-yl)pyrazole (1.15 g, 2.59 mmol, 19.5% yield) as a yellow oil. LCMS: calc. for C₁₉H₂₇BN₂O₂: 326. 2, found: [M+H]⁺327.0. ¹H NMR (400 MHZ, DMSO-d₆) δ 8.01 (s, 1H), 7.59 (s, 1H), 7.15-7.20 (m, 4H), 5.28 (s, 2H), 2.82-2.87 (m, 1H), 1.24 (s, 12H), 1.16 (d, J=8.8 Hz, 6H).

Step 2: To a solution of 1-[(4-isopropylphenyl)methyl]-4-(4,4,5,5-tetramethyl-1,3,2-dioxaborolan-2-yl)pyrazole (31.66 mg, 97.05 μmol, 1 eq) and Int 3-A (30.00 mg, 97.05 μmol, 1 eq) in dioxane (2 mL) and H₂O (0.2 mL) were added Pd(dppf) C₁₂ (7.93 mg, 9.71 μmol, 0.1 eq) and KF (16.92 mg, 291.15 μmol, 6.82 μL, 3 eq). The reaction mixture was stirred at 80° C. for 1 h. After cooling, the reaction mixture was filtered. The filtrate was concentrated, and the residue was purified by prep-HPLC. Most of MeCN was removed under reduced pressure and the solvent was removed by lyophilized to afford product (7.4 mg, impure) as an off-white solid, which was further purified by prep-TLC to give Example 98 (3.4 mg, 7.93 μmol, 8.18% yield) as a white solid. LCMS: calc. for C₂₅H₂₄N₄O₃:428.1, found: [M+H]⁺429.3. ¹H NMR (400 MHZ, DMSO-d₆) δ 11.10 (s, 1H), 8.48 (s, 1H), 8.10 (s, 1H), 7.97 (s, 1H), 7.81 (d, J=8.0 Hz, 1H), 7.62-7.65 (m, 1H), 7.21-7.24 (m, 4H), 5.31 (s, 2H), 4.57 (dd, J=12.0, 4.8 Hz, 1H), 2.65-2.85 (m, 2H), 2.51-2.65 (m, 2H), 2.10-2.25 (m, 1H), 1.17 (d, J=6.8 Hz, 6H).

### Example 238:3-(6-(1-((5-(3, 3-difluoroazetidin-1-yl)pyrimidin-2-yl)methyl)-1H-pyrazol-4-yl)benzo[d]isoxazol-3-yl)piperidine-2,6-dione

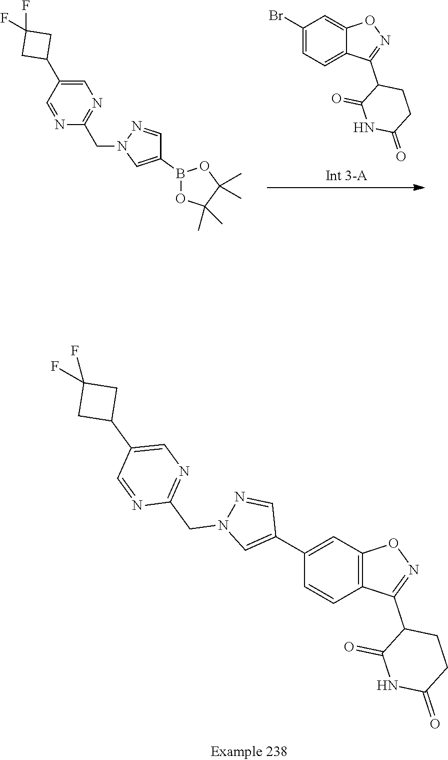 [SMILES: CC1(C)OB(c2cnn(Cc3ncc(C4CC(F)(F)C4)cn3)c2)OC1(C)C ; O=C1CCC(c2noc3cc(Br)ccc23)C(=O)N1 ; O=C1CCC(c2noc3cc(-c4cnn(Cc5ncc(C6CC(F)(F)C6)cn5)c4)ccc23)C(=O)N1]

A mixture of 5-(3,3-difluoroazetidin-1-yl)-2-((4-(4,4,5,5-tetramethyl-1,3,2-dioxaborolan-2-yl)-1H-pyrazol-1-yl)methyl)pyrimidine (80 mg, 212.09 μmol, 1 eq), Int 3-A (65.56 mg, 212.09 μmol, 1 eq), K₃PO₄ (67.53 mg, 318.14 μmol, 1.5 eq), and XPhos Pd G3 (17.95 mg, 21.21 μmol, 0.1 eq) in dioxane (1.6 mL) and H₂O (0.4 mL) was degassed and purged with N₂ for 3 times at 25° C., then the mixture was stirred at 80° C. for 0.5 h under N₂ atmosphere. The residue was concentrated in vacuum. The residue was purified by prep-HPLC and dried by lyophilization to give Example 238 (8 mg, 7.63% yield) as a white solid. ¹H NMR (400 MHZ, DMSO-d₆) § 11.08 (s, 1H), 8.45 (m, 1H), 8.17 (s, 2H), 8.08-8.05 (m, 1H), 7.99-7.97 (m, 1H), 7.83-7.79 (m, 1H), 7.68-7.63 (m, 1H), 5.47-5.43 (m, 2H), 4.61-4.55 (m, 1H), 4.46-4.38 (m, 4H), 2.66 (d, J=4.4 Hz, 1H), 2.63 (d, J=4.4 Hz, 1H), 2.37-2.27 (m, 1H), 2.25-2.17 (m, 1H). LCMS: calc. for C₂₃H₁₉F₂N₇O₃:479.2, found: [M+H]⁺480.4.

### Example 239:3-(6-(1-((5-morpholinopyrazin-2-yl)methyl)-1H-pyrazol-4-yl)benzo[d]isoxazol-3-yl)piperidine-2,6-dione

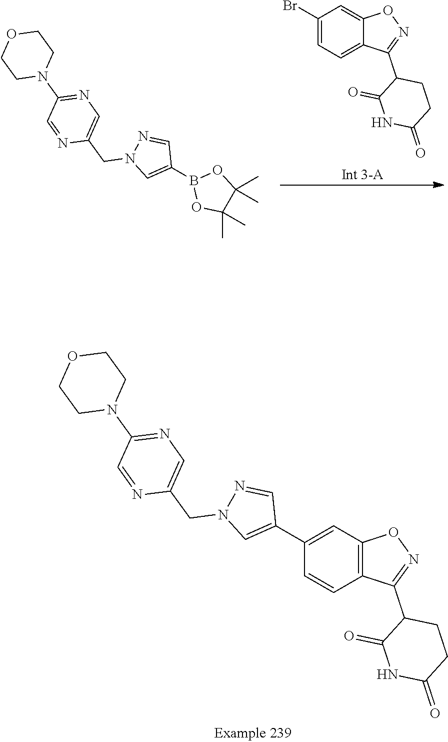 [SMILES: CC1(C)OB(c2cnn(Cc3cnc(N4CCOCC4)cn3)c2)OC1(C)C ; O=C1CCC(c2noc3cc(Br)ccc23)C(=O)N1 ; O=C1CCC(c2noc3cc(-c4cnn(Cc5cnc(N6CCOCC6)cn5)c4)ccc23)C(=O)N1]

A mixture of 4-[5-[[4-(4,4,5,5-tetramethyl-1,3,2-dioxaborolan-2-yl)pyrazol-1-yl]methyl]pyrazin-2-yl]morpholine (50 mg, 134.68 μmol, 1 eq), Int 3-A (41.63 mg, 134.68 μmol, 1 eq), K₃PO₄ (42.88 mg, 202.03 μmol, 1.5 eq), Xphos Pd G3 (17.10 mg, 20.20 μmol, 0.15 eq) in dioxane (0.8 mL) and H₂O (0.2 mL) was degassed and purged with N₂ for 3 times, and then the mixture was stirred at 80° C. for 1 h under N₂ atmosphere. Water (10 mL) was added to the reaction mixture. The reaction mixture was then adjusted to pH˜3 by aq. HCl (1 M). The resulting mixture was extracted with EtOAc (10 mL×3). The combined organic phase was washed with brine (10 mL), dried over anhydrous Na₂SO₄, filtered and concentrated. The crude product was purified by reversed-phase HPLC to give Example 239 (3.9 mg, 5.87% yield) as a white solid. 1HNMR (400 MHZ, DMSO-d₆) § 11.09 (s, 1H), 8.44 (s, 1H), 8.28 (d, J=1.2 Hz, 1H), 8.16 (d, J=1.2 Hz, 1H), 8.08 (s, 1H), 7.97 (s, 1H), 7.80 (d, J=8.4 Hz, 1H), 7.65-7.62 (m, 1H), 5.33 (s, 2H), 4.59-4.55 (m, 1H), 3.71-3.68 (m, 4H), 3.53-3.50 (m, 4H), 2.82-2.72 (m, 1H), 2.65-2.58 (m, 2H), 2.24-2.18 (m, 1H). LCMS: calc. for C₂₄H₂₃N₇O₄:473.2, found: [M+H]⁺474.0.

### Example 429:3-(6-(2-(4-methoxybenzyl) thiazol-5-yl)benzo[d]isoxazol-3-yl)piperidine-2,6-dione

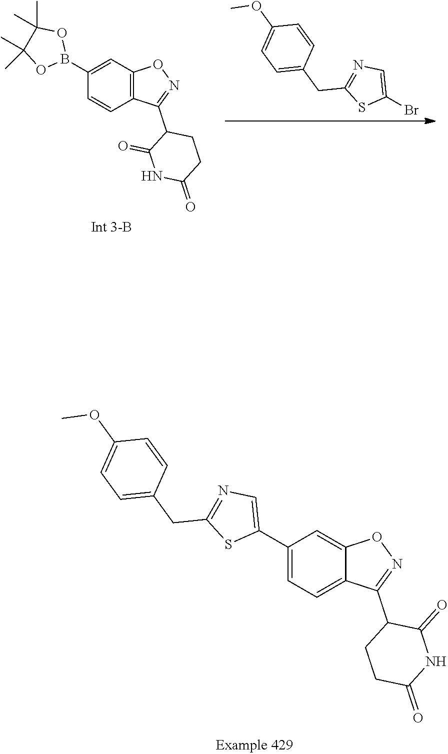 [SMILES: CC1(C)OB(c2ccc3c(C4CCC(=O)NC4=O)noc3c2)OC1(C)C ; COc1ccc(Cc2ncc(Br)s2)cc1 ; COc1ccc(Cc2ncc(-c3ccc4c(C5CCC(=O)NC5=O)noc4c3)s2)cc1]

To a solution of 5-bromo-2-[(4-methoxyphenyl)methyl]thiazole (32.00 mg, 112.62 μmol, 1 eq) and Int 3-B (40.11 mg, 112.62 μmol, 1 eq) in dioxane (2 mL) and H₂O (0.4 mL) was added KF (19.63 mg, 337.86 μmol, 7.91 μL, 3 eq). Then Pd(dppf) C₁₂ (8.24 mg, 11.26 μmol, 0.1 eq) was added into the mixture under N₂ atmosphere. The mixture was stirred at 85° C. for 3 h under N₂ atmosphere. The reaction mixture was poured into water (10 mL) and it was extracted with EtOAc (5 mL×3). The combined organic layer was washed with brine (10 mL), dried over anhydrous Na₂SO₄, filtered and concentrated under reduced pressure to give a residue. The residue was purified by prep-HPLC and dried by lyophilization to afford Example 429 (6.6 mg, 15.07 μmol, 13.38% yield, 99% purity) as a white solid. ¹H NMR (400 MHZ, DMSO-d₆) δ 11.10 (s, 1H), 8.30 (s, 1H), 8.03 (s, 1H), 7.87 (d, J=8.4 Hz, 1H), 7.63 (dd, J=8.4, 1.2 Hz, 1H), 7.31 (d, J=8.4 Hz, 2H), 6.88-6.97 (m, 2H), 4.61 (dd, J=12.0, 4.8 Hz, 1H), 4.30 (s, 2H), 3.74 (s, 3H), 2.70-2.83 (m, 1H), 2.58-2.69 (m, 1H), 2.52-2.57 (m, 1H), 2.15-2.26 (m, 1H). LCMS: calc. for C₂₃H₁₉N₃₀₄S: 433.1, found: [M+H]⁺434.1.

### Example 430:3-[5-fluoro-6-[2-[(4-methoxyphenyl)methyl]oxazol-5-yl]benzofuran-3-yl]piperidine-2,6-dione

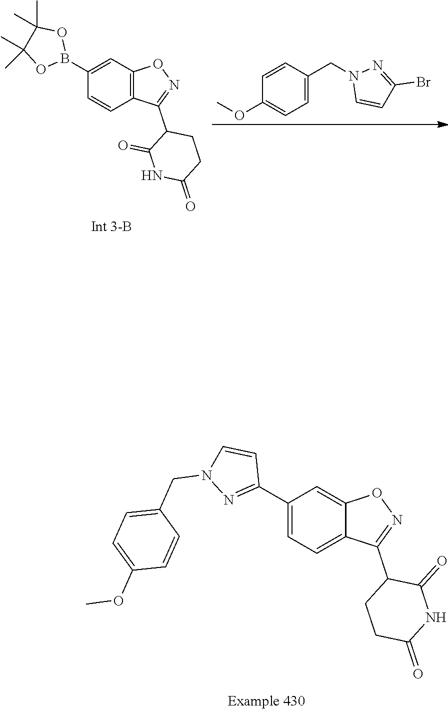 [SMILES: CC1(C)OB(c2ccc3c(C4CCC(=O)NC4=O)noc3c2)OC1(C)C ; COc1ccc(Cn2ccc(Br)n2)cc1 ; COc1ccc(Cn2ccc(-c3ccc4c(C5CCC(=O)NC5=O)noc4c3)n2)cc1]

To a mixture of 3-bromo-1-[(4-methoxyphenyl)methyl]pyrazole (22.50 mg, 84.23 μmol, 1 eq), Int 3-B (30 mg, 84.23 μmol, leq), KF (9.79 mg, 168.45 μmol, 2 eq) and Pd(dppf)Cl₂ (6.16 mg, 8.42 μmol, 0.1 eq) in dioxane (0.8 mL) was added H₂O (0.2 mL). The resulting mixture was degassed and purged with N₂ for 3 times then the mixture was stirred at 85° C. for 16 hours under N₂ atmosphere. The mixture was concentrated under reduce pressure to give residue. The residue was purified by prep-HPLC and dried by lyophilization to afford Example 430 (5.2 mg, 12.00 μmol, 14.2% yield) as an off-white solid. ¹H NMR (400 MHZ, DMSO-d₆) δ 11.10 (s, 1H), 8.09 (s, 1H), 7.79-7.94 (m, 3H), 7.24-7.32 (m, 2H), 6.88-6.96 (m, 3H), 5.32 (s, 2H), 4.56-4.65 (m, 1H), 3.72 (s, 3H), 2.71-2.84 (m, 1H) 2.51-2.68 (m, 2H), 2.17-2.27 (m, 1H). LCMS: calc. for C₂₃H₂ON₄O₄:416.1, found: [M+H]⁺417.0.

Examples 99-118, 240-245, and 431-480 were prepared following the similar procedure that was used for preparing Examples 97-98 and 238-239, starting from Int 3-A and their corresponding boronic acid or ester intermediates, or Example 429-430, starting from Int-3B and their corresponding halides, with proper modifications. Characterizations of Examples 99-118, 240-245, and 431-480 are provided in Table 3.

TABLE 3

| Example ID | Structure | NMR/MS |
|---|---|---|
| 99 | 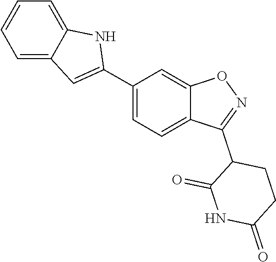 [SMILES: O=C1CCC(c2noc3cc(-c4cc5ccccc5[nH]4)ccc23)C(=O)N1] | ¹H NMR (400 MHz, DMSO-d₆) δ 11.75 (brs, 1H), 11.14 (s, 1H), 8.21 (s, 1H), 7.92 (s, 2H), 7.58 (d, J = 7.6 Hz, 1H), 7.44 (d, J = 8.0 Hz, 1H), 7.11-7.24 (m, 2H), 6.97-7.07 (m, 1H), 4.47-4.75 (m, 1H), 2.70-2.89 (m, 1H), 2.54-2.67 (m, 2H), 2.16-2.29 (m, 1H). LCMS: calc. for C₂₀H₁₅N₃O₃: 345.1, found: [M + H]⁺ 346.1 |
| 100 | 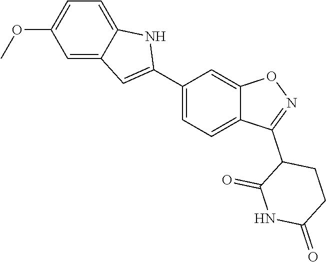 [SMILES: COc1ccc2[nH]c(-c3ccc4c(C5CCC(=O)NC5=O)noc4c3)cc2c1] | ¹H NMR (400 MHz, DMSO-d₆) δ 11.60 (brs, 1H), 11.15 (brs, 1H), 8.17 (s, 1H), 7.90 (s, 2H), 7.32 (d, J = 8.8 Hz, 1H), 7.05-7.10 (m, 2H), 6.80 (dd, J = 8.8, 2.0 Hz, 1H), 4.55-4.65 (m, 1H), 3.77 (s, 3H), 2.73-2.89 (m, 1H), 2.54-2.69 (m, 2H), 2.17-2.27 (m, 1H). LCMS: calc. for C₂₁H₁₇N₃O₄: 375.1, found: [M + H]⁺ 376.2 |
| 101 | 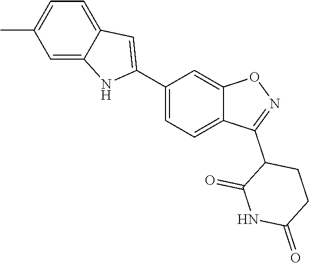 [SMILES: Cc1ccc2cc(-c3ccc4c(C5CCC(=O)NC5=O)noc4c3)[nH]c2c1] | ¹H NMR (400 MHz, DMSO-d₆) δ 11.56 (brs, 1H), 11.13 (brs, 1H), 8.17 (s, 1H), 7.90 (s, 2H), 7.45 (d, J = 8.0 Hz, 1H), 7.22 (s, 1H), 7.09 (s, 1H), 6.87 (d, J = 8.4 Hz, 1H), 4.52-4.70 (m, 1H), 2.71-2.86 (m, 1H), 2.53-2.70 (m, 2H), 2.42 (s, 3H), 2.15-2.28 (m, 1H). LCMS: calc. for C₂₁H₇N₃O₃: 359.1, found: [M + H]⁺ 360.1. |
| 102 | 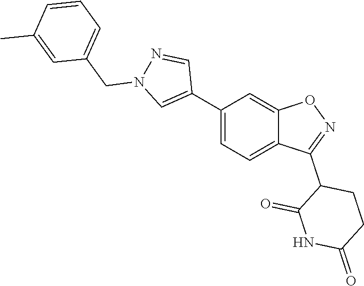 [SMILES: Cc1cccc(Cn2cc(-c3ccc4c(C5CCC(=O)NC5=O)noc4c3)cn2)c1] | ¹H NMR (400 MHz, DMSO-d₆) δ 11.09 (s, 1H), 8.48 (s, 1H), 8.11 (s, 1H), 7.97 (s, 1H), 7.81 (d, J = 8.4 Hz, 1H), 7.64 (dd, J = 8.4, 1.2 Hz, 1H), 7.22-7.27 (m, 1H), 7.09-7.15 (m, 3H), 5.32 (s, 2H), 4.54-4.61 (m, 1H), 2.73-2.78 (m, 1H), 2.62-2.65 (m, 1H), 2.43-2.49 (m, 1H), 2.29 (s, 3H), 2.17-2.24 (m, 1H). LCMS: calc. for C₂₃H₂₀N₄O₃: 400.2, found: [M + H]⁺ 401.0. |
| 103 | 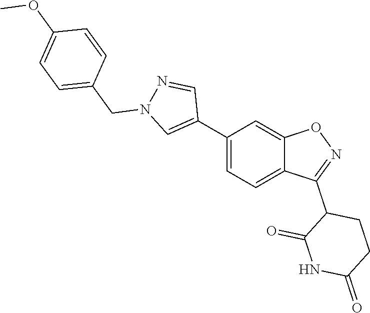 [SMILES: COc1ccc(Cn2cc(-c3ccc4c(C5CCC(=O)NC5=O)noc4c3)cn2)cc1] | ¹H NMR (400 MHz, DMSO-d₆) δ 11.10 (brs, 1H), 8.44 (s, 1H), 8.09 (s, 1H), 7.96 (s, 1H), 7.80 (d, J = 8.0 Hz, 1H), 7.63 (d, J = 8.0 Hz, 1H), 7.27 (d, J = 8.4 Hz, 2H), 6.92 (d, J = 8.4 Hz, 2H), 5.28 (s, 2H), 4.54-4.61 (m, 1H), 3.73 (s, 3H), 2.71-2.83 (m, 1H), 2.55-2.68 (m, 2H), 2.16-2.25 (m, 1H). LCMS: calc. for C₂₃H₂₀N₄O₄: 416.1, found: [M + H]⁺ 417.2. |
| 104 | 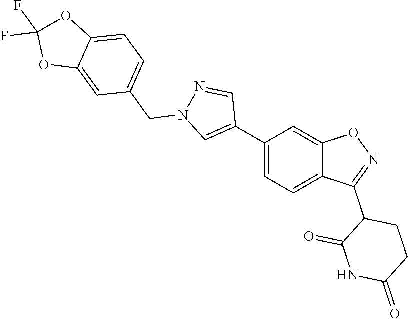 [SMILES: O=C1CCC(c2noc3cc(-c4cnn(Cc5ccc6c(c5)OC(F)(F)O6)c4)ccc23)C(=O)N1] | ¹H NMR (400 MHz, DMSO-d₆) δ 10.91 (s, 1H), 8.35 (s, 1H), 8.00 (s, 1H), 7.86 (s, 1H), 7.79 (s, 1H), 7.55 (d, J = 8.0 Hz, 1H), 7.47 (d, J = 7.6 Hz, 1H), 7.36-7.43 (m, 2H), 7.17 (d, J = 8.0 Hz, 1H), 5.36 (s, 2H), 4.09-4.16 (m, 1H), 2.64-2.87 (m, 1H), 2.54-2.62 (m, 1H), 2.25-2.37 (m, 1H), 2.08-2.16 (m, 1H). LCMS: calc. for C₂₄H₁₇F₂N₃O₅: 465.1, found: [M + H]⁺ 466.2. |
| 105 | 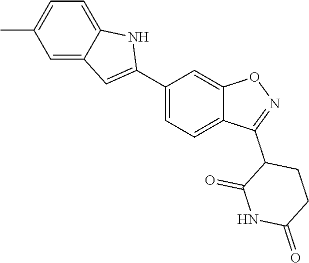 [SMILES: Cc1ccc2[nH]c(-c3ccc4c(C5CCC(=O)NC5=O)noc4c3)cc2c1] | ¹H NMR (400 MHz, DMSO-d₆) δ 11.60 (brs, 1H), 11.13 (brs, 1H), 8.18 (s, 1H), 7.90 (s, 2H), 7.30-7.37 (m, 2H), 7.06 (d, J = 1.2 Hz, 1H), 6.98 (dd, J = 8.4, 1.2 Hz, 1H), 4.58-4.64 (m, 1H), 2.73-2.85 (m, 1H), 2.53-2.68 (m, 2H), 2.38 (s, 3H), 2.19-2.26 (m, 1H). LCMS: calc. for C₂₁H₁₇N₃O₃: 359.1, found: [M + H]⁺ 360.2 |
| 106 | 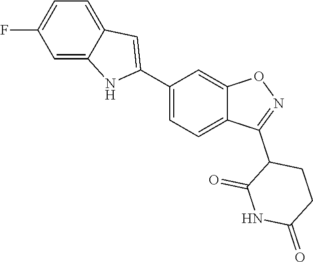 [SMILES: O=C1CCC(c2noc3cc(-c4cc5ccc(F)cc5[nH]4)ccc23)C(=O)N1] | ¹H NMR (400 MHz, DMSO-d₆) δ 11.88 (brs, 1H), 11.13 (brs, 1H), 8.19 (s, 1H), 7.87-7.94 (m, 2H), 7.54-7.61 (m, 1H), 7.16-7.22 (m, 2H), 6.87-6.94 (m, 1H), 4.58-4.65 (m, 1H), 2.72-2.85 (m, 1H), 2.53-2.68 (m, 2H), 2.18-2.28 (m, 1H). LCMS: calc. for C₂₀H₁₄FN₃O₃: 363.1, found: [M + H]⁺ 364.1 |
| 107 | 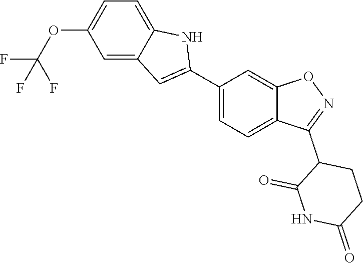 [SMILES: O=C1CCC(c2noc3cc(-c4cc5cc(OC(F)(F)F)ccc5[nH]4)ccc23)C(=O)N1] | ¹H NMR (400 MHz, DMSO-d₆) δ 12.06 (brs, 1H), 11.14 (brs, 1H), 8.24 (s, 1H), 7.94 (d, J = 2.4 Hz, 2H), 7.57 (s, 1H), 7.52 (d, J = 8.8 Hz, 1H), 7.23 (s, 1H), 7.13 (d, J = 8.8 Hz, 1H), 4.55- 4.65 (m, 1H), 2.73-2.85 (m, 1H), 2.54-2.67 (m, 2H), 2.15-2.29 (m, 1H). LCMS: calc. for C₂₁H₁₄F₃N₃O₄: 429.1, found: [M + H]⁺ 430.2 |
| 108 | 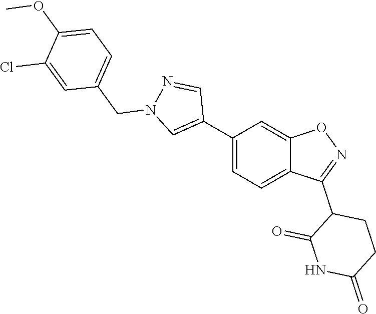 [SMILES: COc1ccc(Cn2cc(-c3ccc4c(C5CCC(=O)NC5=O)noc4c3)cn2)cc1Cl] | ¹H NMR (400 MHz, DMSO-d₆) δ 11.10 (brs, 1H), 8.47 (s, 1H), 8.11 (s, 1H), 7.97 (s, 1H), 7.81 (d, J = 8.0 Hz, 1H), 7.61-7.65 (m, 1H), 7.41 (d, J = 2.0 Hz, 1H), 7.39-7.44 (m, 1H), 7.14 (d, J = 8.8 Hz, 1H), 5.30 (s, 2H), 4.54-4.60 (m, 1H), 3.83 (s, 3H), 2.72-2.82 (m, 1H), 2.53- 2.65 (m, 2H), 2.17-2.24 (m, 1H). LCMS: calc. for C₂₃H₁₉ClN₄O₄: 450.1, found: [M + H]⁺ 451.2. |
| 109 | 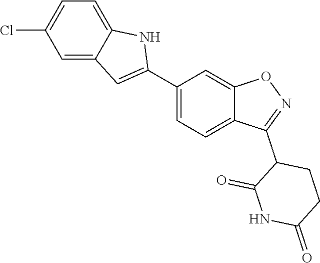 [SMILES: O=C1CCC(c2noc3cc(-c4cc5cc(Cl)ccc5[nH]4)ccc23)C(=O)N1] | ¹H NMR (400 MHz, DMSO-d₆) δ 11.95 (brs, 1H), 11.13 (brs, 1H), 8.22 (s, 1H), 7.90-7.97 (m, 2H), 7.63 (d, J = 1.6 Hz, 1H), 7.45 (d, J = 8.8 Hz, 1H), 7.12-7.18 (m, 2H), 4.59-4.66 (m, 1H), 2.74-2.84 (m, 1H), 2.52-2.63 (m, 2H), 2.19-2.26 (m, 1H). LCMS: calc. for C₂₀H₁₄ClN₃O₃: 379.1, found: [M +H]⁺ 380.1. |
| 110 | 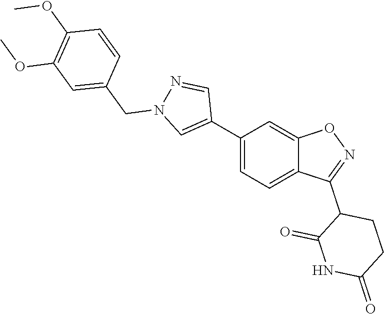 [SMILES: COc1ccc(Cn2cc(-c3ccc4c(C5CCC(=O)NC5=O)noc4c3)cn2)cc1OC] | ¹H NMR (400 MHz, DMSO-d₆) δ 11.10 (s, 1H), 8.45 (s, 1H), 8.10 (s, 1H), 7.97 (s, 1H), 7.80 (d, J = 8.4 Hz, 1H), 7.61-7.66 (m, 1H), 6.97-7.01 (m, 1H), 6.90-6.96 (m, 1H), 6.81-6.87 (m, 1H), 5.27 (s, 2H), 4.54-4.61 (m, 1H), 3.74 (s, 3H), 3.73 (s, 3H), 2.71-2.83 (m, 1H), 2.57-2.65 (m, 1H), 2.53-2.57 (m, 1H), 2.15-2.25 (m, 1H). LCMS: calc. for C₂₄H₂₂N₄O₅: 446.2, found: [M + H]⁺ 447.0 |
| 111 | 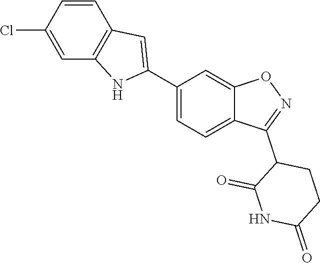 [SMILES: O=C1CCC(c2noc3cc(-c4cc5ccc(Cl)cc5[nH]4)ccc23)C(=O)N1] | ¹H NMR (400 MHz, DMSO-d₆) δ 11.92 (brs, 1H), 11.13 (brs, 1H), 8.21 (s, 1H), 7.88-7.98 (m, 2H), 7.60 (d, J = 8.4 Hz, 1H), 7.44 (s, 1H), 7.20 (s, 1H), 7.02-7.11 (m, 1H), 4.59-4.66 (m, 1H), 2.74-2.85 (m, 1H), 2.53-2.62 (m, 2H), 2.20-2.27 (m, 1H). LCMS: calc. for C₂₀H₁₄ClN₃O₃: 379.1, found: [M + H]⁺ 380.1. |
| 112 | 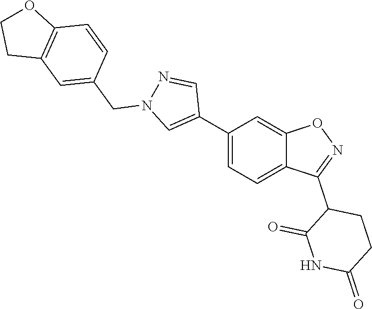 [SMILES: O=C1CCC(c2noc3cc(-c4cnn(Cc5ccc6c(c5)CCO6)c4)ccc23)C(=O)N1] | ¹H NMR (400 MHz, DMSO-d₆) δ 11.10 (s, 1H), 8.44 (s, 1H), 8.09 (s, 1H), 7.96 (s, 1H), 7.81 (d, J = 8.4 Hz, 1H), 7.60-7.67 (m, 1H), 7.22 (s, 1H), 7.10 (d, J = 8.4 Hz, 1H), 6.74 (d, J = 8.4 Hz, 1H), 5.25 (s, 2H), 4.55-4.65 (m, 1H), 4.51 (t, J = 8.8 Hz, 2H), 3.16 (t, J = 8.4 Hz, 2H), 2.72-2.83 (m, 1H), 2.55-2.65 (m, 2H), 2.15-2.25 (m, 1H). LCMS: calc. for C₂₄H₂₀N₄O₄: 428.1, found: [M + H]⁺ 428.4 |
| 113 | 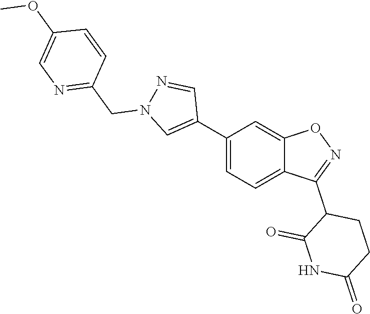 [SMILES: COc1ccc(Cn2cc(-c3ccc4c(C5CCC(=O)NC5=O)noc4c3)cn2)nc1] | ¹H NMR (400 MHz, DMSO-d₆) δ 11.10 (brs, 1H), 8.47 (s, 1H), 8.25 (d, J = 2.8 Hz, 1H), 8.11 (s, 1H), 7.98 (s, 1H), 7.81 (d, J = 8.4 Hz, 1H), 7.65 (d, J = 9.2 Hz, 1H), 7.39 (dd, J = 8.8, 3.2 Hz, 1H), 7.22 (d, J = 8.4 Hz, 1H), 5.39 (s, 2H), 4.63-4.53 (m, 1H), 3.81 (s, 3H), 2.85-2.60 (m, 3H), 2.24-2.18 (m, 1H). LCMS: calc. for C₂₂H₁₉N₅O₄: 417.1, found: [M + H]⁺ 418.0 |
| 114 | 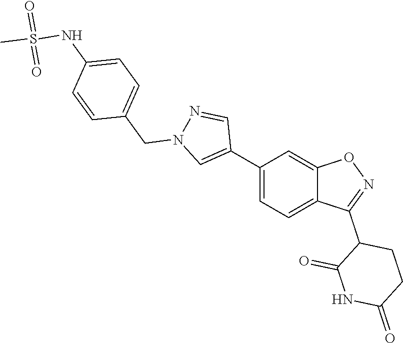 [SMILES: CS(=O)(=O)Nc1ccc(Cn2cc(-c3ccc4c(C5CCC(=O)NC5=O)noc4c3)cn2)cc1] | ¹H NMR (400 MHz, DMSO-d₆) δ 11.10 (s, 1H), 9.77 (brs, 1H), 8.47 (s, 1H), 8.10 (s, 1H), 7.96 (s, 1H), 7.81 (d, J = 8.4 Hz, 1H), 7.63 (d, J = 8.4 Hz, 1H), 7.29 (d, J = 8.4 Hz, 2H), 7.19 (d, J = 8.4 Hz, 2H), 5.31 (s, 2H), 4.57 (dd, J = 12.0, 5.2 Hz, 1H), 2.97 (s, 3H), 2.72-2.82 (m, 1H), 2.57- 2.65 (m, 1H), 2.44-2.48 (m, 1H), 2.16-2.25 (m, 1H). LCMS: calc. for C₂₃H₂₁N₅O₅S: 479.1, found: [M + H]⁺ 480.2 |
| 115 | 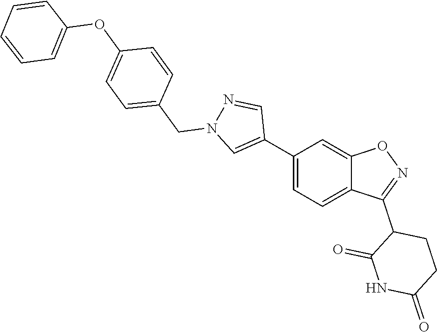 [SMILES: O=C1CCC(c2noc3cc(-c4cnn(Cc5ccc(Oc6ccccc6)cc5)c4)ccc23)C(=O)N1] | ¹H NMR (400 MHz, DMSO-d₆) δ 11.08 (s, 1H), 8.49 (s, 1H), 8.11 (s, 1H), 7.97 (s, 1H), 7.81 (d, J = 8.4 Hz, 1H), 7.64 (d, J = 8.4 Hz, 1H), 7.33- 7.40 (m, 4H), 7.13 (t, J = 7.6 Hz, 1H), 7.00 (d, J = 7.6 Hz, 4H), 5.35 (s, 2H), 4.55-4.59 (m, 1H), 2.72-2.76 (m, 1H), 2.62-2.64 (m, 2H), 2.18-2.23 (m, 1H). LCMS: calc. for C₂₈H₂₂N₄O₄: 478.2, found: [M + H]⁺ 479.1 |
| 116 | 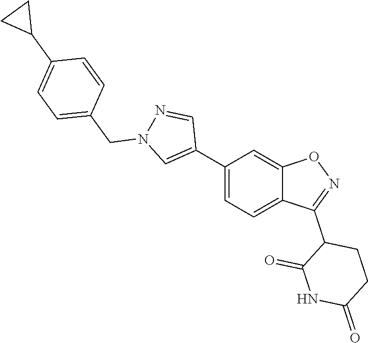 [SMILES: O=C1CCC(c2noc3cc(-c4cnn(Cc5ccc(C6CC6)cc5)c4)ccc23)C(=O)N1] | ¹H NMR (400 MHz, DMSO-d₆) δ 11.10 (brs, 1H), 8.46 (s, 1H), 8.09 (s, 1H), 7.96 (s, 1H), 7.80 (d, J = 8.4 Hz, 1H), 7.63 (d, J = 8.8 Hz, 1H), 7.19 (d, J = 8.4 Hz, 2H), 7.06 (d, J = 8.0 Hz, 2H), 5.29 (s, 2H), 4.54-4.60 (m, 1H), 2.71- 2.82 (m, 1H), 2.53-2.65 (m, 2H), 2.16-2.25 (m, 1H), 1.85-1.93 (m, 1H), 0.89-0.95 (m, 2H), 0.61-0.66 (m, 2H). LCMS: calc. for C₂₅H₂₂N₄O₃: 426.2, found: [M + H]⁺ 427.1 |
| 117 | 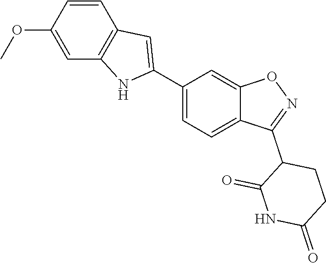 [SMILES: COc1ccc2cc(-c3ccc4c(C5CCC(=O)NC5=O)noc4c3)[nH]c2c1] | ¹H NMR (400 MHz, DMSO-d₆) δ 11.56 (brs, 1H), 11.12 (brs, 1H), 8.12 (s, 1H), 7.87 (s, 2H), 7.45 (d, J = 8.4 Hz, 1H), 7.08 (s, 1H), 6.90 (s, 1H), 6.70 (dd, J = 8.8, 2.0 Hz, 1H), 4.41-4.71 (m, 1H), 3.81 (s, 3H), 2.72-2.89 (m, 1H), 2.58- 2.70 (m, 2H), 2.08-2.28 (m, 1H). LCMS: calc. for C₂₁H₁₇N₃O_{4:} 375.1, found: [M + H]⁺ 376.0 |
| 118 | 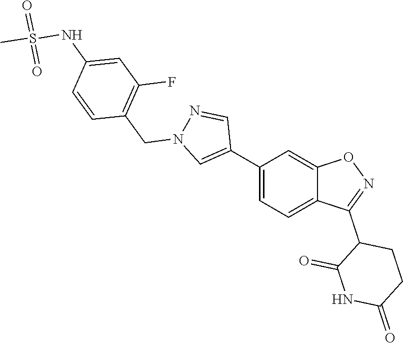 [SMILES: CS(=O)(=O)Nc1ccc(Cn2cc(-c3ccc4c(C5CCC(=O)NC5=O)noc4c3)cn2)c(F)c1] | ¹H NMR (400 MHz, DMSO-d₆) δ 11.09 (s, 1H), 9.95-10.25 (m, 1H), 8.44 (s, 1H), 8.10 (s, 1H), 7.97 (s, 1H), 7.80 (d, J = 8.4 Hz, 1H), 7.64 (dd, J = 8.4, 0.8 Hz, 1H), 7.31 (t, J = 8.4 Hz, 1H), 6.94-7.07 (m, 2H), 5.35 (s, 2H), 4.50-4.63 (m, 1H), 3.04 (s, 3H), 2.70-2.87 (m, 1H), 2.54-2.67 (m, 2H), 2.15-2.25 (m, 1H). LCMS: calc. for C₂₃H₂₀FN₅O₅S: 497.1, found: [M + H]⁺ 498.1 |
| 240 | 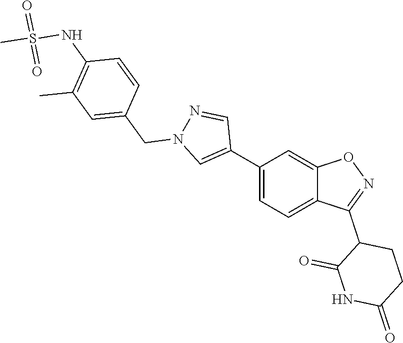 [SMILES: Cc1cc(Cn2cc(-c3ccc4c(C5CCC(=O)NC5=O)noc4c3)cn2)ccc1NS(C)(=O)=O] | ¹H NMR (400 MHz, DMSO-d₆) δ 11.11 (s, 1H), 8.48 (s, 1H), 8.39 (s, 0.4H, FA), 8.10 (s, 1H), 7.97 (s, 1H), 7.81 (d, J = 8.4 Hz, 1H), 7.62-7.65 (m, 1H), 7.25 (d, J = 8.0 Hz, 1H), 7.19 (s, 1H), 7.19 (s, 1H), 7.13 (d, J = 1.6 Hz, 1H), 5.30 (s, 2H), 4.55-4.60 (m, 1H), 2.94 (s, 3H), 2.66-2.82 (m, 1H), 2.55-2.62 (m, 2H), 2.28 (s, 3H), 2.19- 2.23 (m, 1H). LCMS: calc. for C₂₄H₂₃N₅O₆S: 493.1, found: [M + H]⁺ 494.2. |
| 241 | 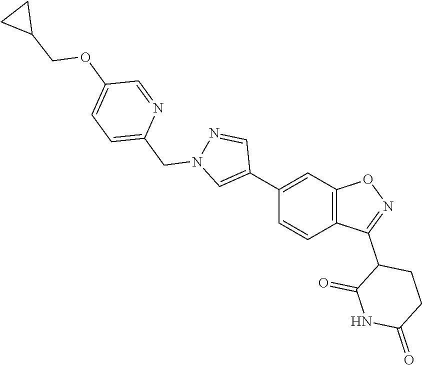 [SMILES: O=C1CCC(c2noc3cc(-c4cnn(Cc5ccc(OCC6CC6)cn5)c4)ccc23)C(=O)N1] | ¹H NMR (400 MHz, DMSO-d₆) δ 11.15-11.01 (m, 1H), 8.52-8.42 (m, 1H), 8.26-8.21 (m, 1H), 8.10 (s, 1H), 8.00-7.94 (m, 1H), 7.84- 7.77 (m, 1H), 7.68-7.61 (m, 1H), 7.41-7.34 (m, 1H), 7.24-7.14 (m, 1H), 5.41-5.34 (m, 2H), 4.62-4.53 (m, 1H), 3.88 (d, J = 7.6 Hz, 2H), 2.80-2.71 (m, 2H), 2.28-2.15 (m, 1H), 1.29-1.17 (m, 2H), 0.62-0.52 (m, 2H), 0.35- 0.29 (m, 2H). MS: (ESI) m/z calculated for C₂₅H₂₃N₅O₄ [M + H]⁺ 458.1, found 458.1. |
| 242 | 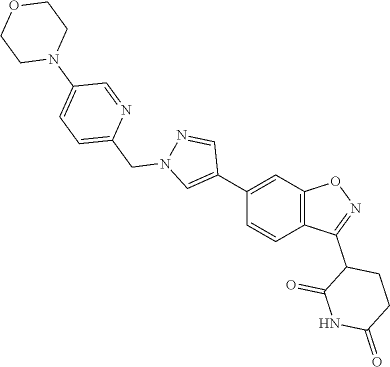 [SMILES: O=C1CCC(c2noc3cc(-c4cnn(Cc5ccc(N6CCOCC6)cn5)c4)ccc23)C(=O)N1] | ¹H NMR (400 MHz, DMSO-d₆) δ 11.15-11.06 (m, 1H), 8.50-8.47 (m, 1H), 8.46 (s, 1H), 8.26 (d, J = 2.8 Hz, 1H), 8.10 (s, 1H), 7.98 (s, 1H), 7.81 (d, J = 8.4 Hz, 1H), 7.6-7.64 (m, 1H), 7.35-7.30 (m, 1H), 7.16-7.10 (m, 1H), 5.34 (s, 2H), 4.60-4.55 (m, 1H), 3.76-3.71 (m, 4H), 3.18-3.13 (m, 4H), 2.82-2.72 (m, 2H), 2.64 (t, J = 4.0 Hz, 1H), 2.24-2.19 (m, 1H). MS: (ESI) m/z calculated for C₂₅H₂₄N₆O₄ [M + H]⁺ 473.1, found 473.1. |
| 243 | 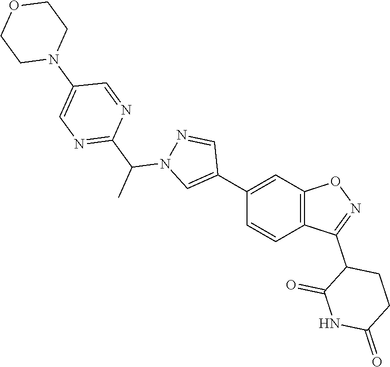 [SMILES: CC(c1ncc(N2CCOCC2)cn1)n1cc(-c2ccc3c(C4CCC(=O)NC4=O)noc3c2)cn1] | ¹HNMR (400 MHz, DMSO-d₆) δ 11.09 (br s, 1H), 8.53 (s, 1H), 8.25 (d, J = 2.8 Hz, 1H), 8.08 (s, 1H), 7.99 (s, 1H), 7.80 (d, J = 8.4 Hz, 1H), 7.67-7.65 (m, 1H), 7.32-7.30 (m, 1H), 7.07 (d, J = 8.8 Hz, 1H), 5.63-5.56 (m, 1H), 4.59- 4.55 (m, 1H), 3.74-3.71 (m, 4H), 3.15-3.12 (m, 4H), 2.82-2.72 (m, 1H), 2.65-2.62 (m, 1H), 2.61-2.57 (m, 1H), 2.24-2.17 (m, 1H), 1.84 (d, J = 7.2 Hz, 3H). MS: (ESI) m/z calculated for C₂₆H₂₆N₆O₄ [M + H]⁺ 487.2, found 487.2. |
| 244 | 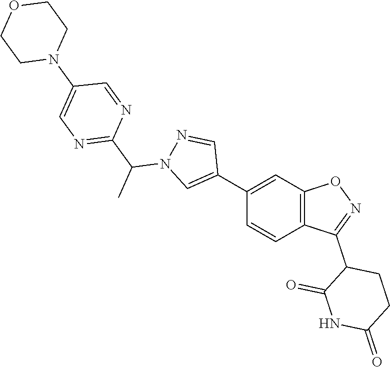 | ¹H NMR (400 MHz, DMSO-d₆) δ 11.10 (s, 1H), 8.55 (s, 2H), 8.48 (s, 1H), 8.07 (s, 1H), 7.98 (s, 1H), 7.82 (d, J = 8.4 Hz, 1H), 7.67- 7.65 (m, 1H), 5.52 (s, 2H), 4.60-4.56 (m, 1H), 3.90 (s, 3H), 2.78-2.75 (m, 1H), 2.64-2.63 (m, 1H), 2.54-2.50 (m, 1H), 2.24-2.20 (m, 1H). MS: (ESI) m/z calculated for C₂₁H₁₈N₆O₄ [M + H]⁺ 419.1, found 419.1. |
| 245 | 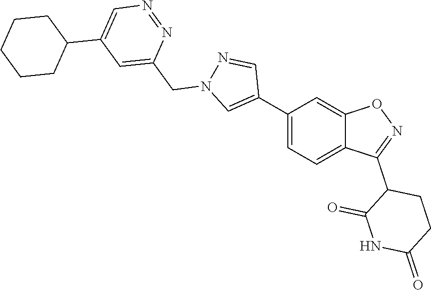 [SMILES: O=C1CCC(c2noc3cc(-c4cnn(Cc5cc(C6CCCCC6)cnn5)c4)ccc23)C(=O)N1] | ¹H NMR (400 MHz, DMSO-d₆) δ 11.10 (s, 1H), 9.15 (d, J = 2.0 Hz, 1H), 8.58 (s, 1H), 8.16 (s, 1H), 8.00 (s, 1H), 7.82 (d, J = 8.4 Hz, 1H), 7.66 (d, J = 8.0 Hz, 1H), 7.42 (d, J = 2.0 Hz, 1H), 5.66 (s, 2H), 4.58 (dd, J = 11.6, 4.8 Hz, 1H), 2.72-2.82 (m, 1H), 2.52-2.68 (m, 3H), 2.17-2.25 (m, 1H), 1.73-1.82 (m, 4H), 1.65-1.72 (m, 1H), 1.31-1.42 (m, 4H), 1.16-1.26 (m, 1H). LCMS: calc. for C₂₆H₂₆N₆O₃: 470.2, found: [M + H]⁺ 471.2. |
| 431 | 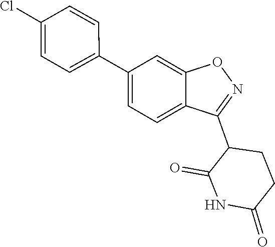 [SMILES: O=C1CCC(c2noc3cc(-c4ccc(Cl)cc4)ccc23)C(=O)N1] | ¹H NMR (400 MHz, DMSO-d₆) δ 11.13 (s, 1H), 8.04-8.07 (m, 1H), 7.92-7.99 (m, 1H), 7.82-7.85 (m, 2H), 7.72-7.77 (m, 1H), 7.56- 7.59 (m, 2H), 4.61-4.65 (m, 1H), 2.75-2.80 (m, 1H), 2.56-2.65 (m, 2H), 2.20-2.33 (m, 1H). LCMS: calc. for C₁₈H₁₃ClN₂O₃: 340.1, found: [M + H]⁺ 341.1. |
| 432 | 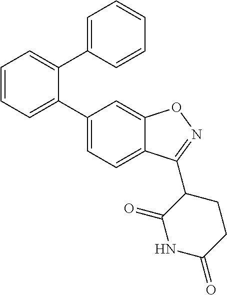 [SMILES: O=C1CCC(c2noc3cc(-c4ccccc4-c4ccccc4)ccc23)C(=O)N1] | ¹H NMR (400 MHz, DMSO-d₆) δ 10.07 (s, 1H), 7.67 (d, J = 8.0 Hz, 1H), 7.51-7.54 (m, 3H), 7.42-7.46 (m, 1H), 7.23-7.30 (m, 3H), 7.13-7.16 (m, 2H), 7.06-7.09 (m, 1H), 4.54- 4.59 (m, 1H), 2.71-2.75 (m, 1H), 2.57-2.63 (m, 1H), 2.45-2.47 (m, 1H), 2.16-2.22 (m, 1H). LCMS: calc. for C₂₄H₁₈N₂O₃: 382.1, found: [M + H]⁺ 383.1. |
| 433 | 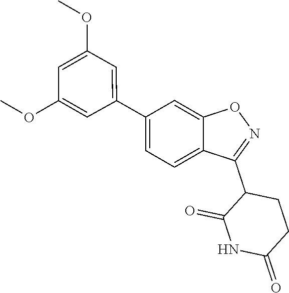 [SMILES: COc1cc(OC)cc(-c2ccc3c(C4CCC(=O)NC4=O)noc3c2)c1] | ¹H NMR (400 MHz, DMSO-d₆) δ 11.12 (brs, 1H), 8.07 (s, 1H), 7.91 (d, J = 8.4 Hz, 1H), 7.72 (d, J = 8.4 Hz, 1H), 6.92 (d, J = 2.0 Hz, 2H), 6.55-6.60 (m, 1H), 4.60-4.65 (m, 1H), 3.83 (s, 6H), 2.75-2.82 (m, 1H), 2.55-2.65 (m, 2H), 2.15-2.25 (m, 1H). LCMS: calc. for C₂₀H₁₈N₂O₅: 366.1, found: [M + H]⁺ 367.2 |
| 434 | 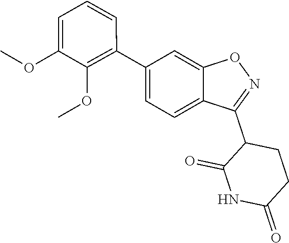 [SMILES: COc1cccc(-c2ccc3c(C4CCC(=O)NC4=O)noc3c2)c1OC] | ¹H NMR (400 MHz, DMSO-d₆) δ 11.11 (brs, 1H), 7.89 (d, J = 8.4 Hz, 1H), 7.80 (s, 1H), 7.48 (dd, J = 8.4, 1.2 Hz, 1H), 7.08- 7.24 (m, 2H), 7.00 (d, J = 7.2 Hz, 1H), 4.63 (dd, J = 11.6, 4.8 Hz, 1H), 3.87 (s, 3H), 3.57 (s, 3H), 2.74-2.85 (m, 1H), 2.54-2.63 (m, 2H), 2.20-2.27 (m, 1H). LCMS: calc. for C₂₀H₁₈N₂O₅: 366.1, found: [M + H]⁺ 367.1. |
| 435 | 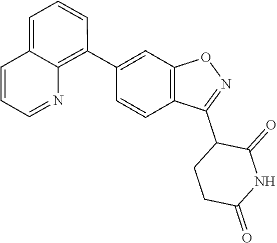 [SMILES: O=C1CCC(c2noc3cc(-c4cccc5cccnc45)ccc23)C(=O)N1] | ¹H NMR (400 MHz, DMSO-d₆) δ 11.15 (brs, 1H), 8.92 (dd, J = 8.4, 1.6 Hz, 1H), 8.43-8.47 (m, 1H), 8.08 (d, J = 8.0 Hz, 1H), 7.85-7.95 (m, 3H), 7.74 (t, J = 8.0 Hz, 1H), 7.52-7.65 (m, 2H), 4.60-4.70 (m, 1H), 2.75- 2.85 (m, 1H), 2.55-2.65 (m, 2H), 2.17-2.27 (m, 1H). LCMS: calc. for C₂₁H₁₅N₃O₃: 357.1, found: [M + H]⁺ 358.2. |
| 436 | 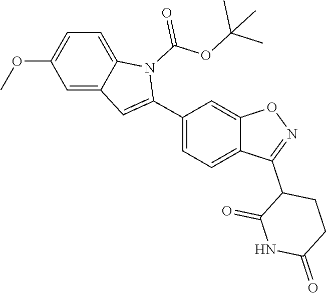 [SMILES: COc1ccc2c(c1)cc(-c1ccc3c(C4CCC(=O)NC4=O)noc3c1)n2C(=O)OC(C)(C)C] | ¹H NMR (400 MHz, DMSO-d₆) δ 11.12 (brs, 1H), 8.00 (d, J = 9.2 Hz, 1H), 7.86- 7.92 (m, 2H), 7.44-7.50 (m, 1H), 7.17 (d, J = 2.4 Hz, 1H), 6.98 (dd, J = 9.2, 2.4 Hz, 1H), 6.81 (s, 1H), 4.58-4.70 (m, 1H), 3.80 (s, 3H), 2.73-2.88 (m, 1H), 2.55-2.69 (m, 2H), 2.19-2.28 (m, 1H), 1.25 (s, 9H). LCMS: calc. for C₂₆H₂₅N₃O₆: 475.2, found: [M + H]⁺ 476.2. |
| 437 | 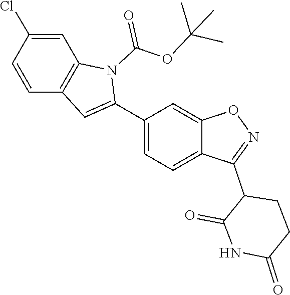 [SMILES: CC(C)(C)OC(=O)n1c(-c2ccc3c(C4CCC(=O)NC4=O)noc3c2)cc2ccc(Cl)cc21] | ¹H NMR (400 MHz, DMSO-d₆) δ 11.14 (brs, 1H), 8.15 (d, J = 1.2 Hz, 1H), 7.87- 7.96 (m, 2H), 7.69 (d, J = 8.0 Hz, 1H), 7.50 (d, J = 8.0 Hz, 1H), 7.35 (d, J = 8.0 Hz, 1H), 6.92 (s, 1H), 4.62-4.69 (m, 1H), 2.73- 2.86 (m, 1H), 2.52-2.63 (m, 2H), 2.17-2.28 (m, 1H), 1.23 (s, 9H). LCMS: calc. for C₂₅H₂₂ClN₃O₅: 479.1, found: [M + H]⁺ 480.1. |
| 438 | 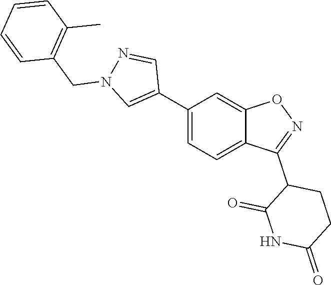 [SMILES: Cc1ccccc1Cn1cc(-c2ccc3c(C4CCC(=O)NC4=O)noc3c2)cn1] | ¹H NMR (400 MHz, DMSO-d₆) δ 11.11 (brs, 1H), 8.42 (s, 1H), 8.12 (s, 1H), 7.98 (s, 1H), 7.80 (d, J = 8.4 Hz, 1H), 7.64 (d, J = 8.4 Hz, 1H), 7.16-7.23 (m, 3H), 7.03 (d, J = 7.2 Hz, 1H), 5.38 (s, 2H), 4.56-4.61 (m, 1H), 2.72-2.82 (m, 1H), 2.54-2.64 (m, 2H), 2.34 (s, 3H), 2.17-2.24 (m, 1H). LCMS: calc. for C₂₃H₂₀N₄O₃: 400.2, found: [M + H]⁺ 401.1. |
| 439 | 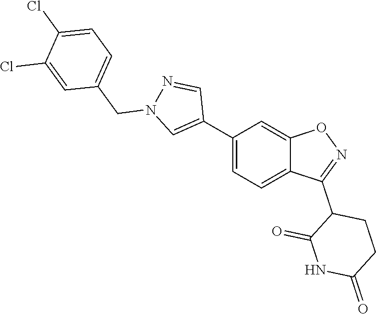 [SMILES: O=C1CCC(c2noc3cc(-c4cnn(Cc5ccc(Cl)c(Cl)c5)c4)ccc23)C(=O)N1] | ¹H NMR (400 MHz, DMSO-d₆) δ 11.09 (s, 1H), 8.51 (s, 1H), 8.15 (s, 1H), 7.98 (s, 1H), 7.82 (d, J = 8.4 Hz, 1H), 7.56-7.67 (m, 3H), 7.28 (dd, J = 8.4, 2.0 Hz, 1H), 5.40 (s, 2H), 4.58 (dd, J = 12.0, 4.8 Hz, 1H), 2.75-2.80 (m, 1H), 2.59-2.69 (m, 2H), 2.18-2.26 (m, 1H). LCMS: calc. for C22H16C12N403: 454.1, found: [M + H]⁺ 454.9. |
| 440 | 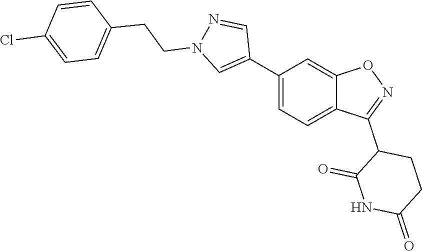 [SMILES: O=C1CCC(c2noc3cc(-c4cnn(CCc5ccc(Cl)cc5)c4)ccc23)C(=O)N1] | ¹H NMR (400 MHz, DMSO-d₆) δ 11.10 (s, 1H), 8.30 (s, 1H), 8.08 (s, 1H), 7.92 (s, 1H), 7.80 (d, J = 8.4 Hz, 1H), 7.59 (dd, J = 8.4, 1.2 Hz, 1H), 7.29-7.38 (m, 2H), 7.19-7.24 (m, 2H), 4.53-4.63 (m, 1H), 4.38 (t, J = 7.2 Hz, 2H), 3.17 (t, J = 7.2 Hz, 2H), 2.71-2.86 (m, 1H), 2.62-2.65 (m, 1H), 2.58-2.62 (m, 1H), 2.16-2.26 (m, 1H). LCMS: calc. for C₂₃H₁₉ClN₄O₃: 434.1, found: [M + H]⁺ 435.1. |
| 441 | 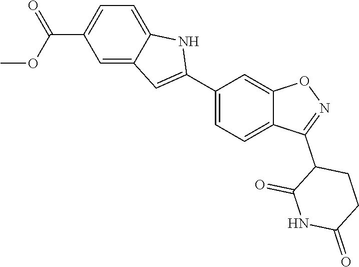 [SMILES: COC(=O)c1ccc2[nH]c(-c3ccc4c(C5CCC(=O)NC5=O)noc4c3)cc2c1] | ¹H NMR (400 MHz, DMSO-d₆) δ 12.15 (brs, 1H), 11.14 (brs, 1H), 8.27 (s, 1H), 8.08 (s, 2H), 7.92-8.05 (m, 2H), 7.55-7.72 (m, 2H), 7.27 (s, 1H), 4.50-4.68 (m, 1H), 3.88 (s, 3H), 2.70-2.88 (m, 1H), 2.50-2.68 (m, 2H), 2.15-2.26 (m, 1H). LCMS: calc. for C₂₂H₁₇N₃O₅: 403.1, found: [M + H]⁺ 404.1. |
| 442 | 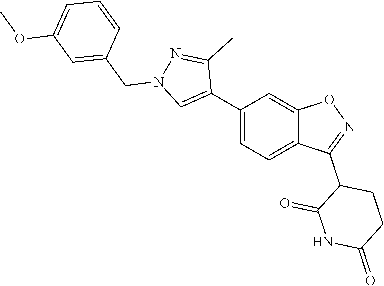 [SMILES: COc1cccc(Cn2cc(-c3ccc4c(C5CCC(=O)NC5=O)noc4c3)c(C)n2)c1] | ¹H NMR (400 MHz, DMSO-d₆) δ 11.10 (s, 1H), 8.25 (s, 1H), 7.83-7.88 (m, 1H), 7.77 (s, 1H), 7.49 (d, J = 8.4 Hz, 1H), 7.28 (t, J = 8.0 Hz, 1H), 6.85-6.91 (m, 2H), 6.72-6.79 (m, 1H), 5.36 (s, 1H), 5.20 (s, 2H), 4.60 (dd, J = 12.0, 4.8 Hz, 1H), 3.73-3.75 (m, 2H), 3.71-3.77 (m, 1H), 2.73-2.83 (m, 1H), 2.63- 2.68 (m, 1H), 2.60 (m, 1H), 2.38 (s, 1H), 2.37-2.43 (m, 1H), 2.20-2.26 (m, 1H). LCMS: calc. for C₂₄H₂₂N₄O₄: 430.2, found: [M + H]⁺ 431.0. |
| 443 | 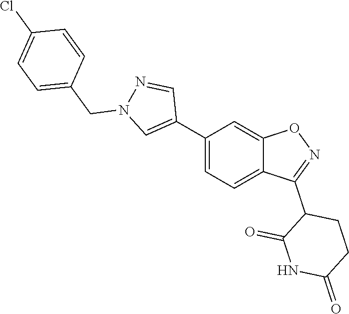 [SMILES: O=C1CCC(c2noc3cc(-c4cnn(Cc5ccc(Cl)cc5)c4)ccc23)C(=O)N1] | ¹H NMR (400 MHz, DMSO-d₆) δ 11.09 (s, 1H), 8.49 (s, 1H), 8.13 (s, 1H), 7.97 (s, 1H), 7.81 (d, J = 8.4 Hz, 1H), 7.64 (d, J = 8.4 Hz, 1H), 7.43 (d, J = 8.4 Hz, 2H), 7.32 (d, J = 8.4 Hz, 2H), 5.38 (s, 2H), 4.54-4.60 (m, 1H), 2.75-2.79 (m, 1H), 2.63-2.65 (m, 2H), 2.18-2.23 (m, 1H). LCMS: calc. for C₂₂H₁₇ClN₄O₃: 420.1, found: [M + H]⁺ 420.9. |
| 444 | 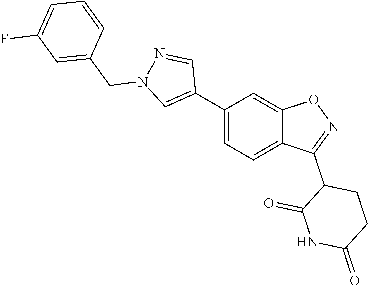 [SMILES: O=C1CCC(c2noc3cc(-c4cnn(Cc5cccc(F)c5)c4)ccc23)C(=O)N1] | ¹H NMR (400 MHz, DMSO-d₆) δ 11.10 (s, 1H), 8.51 (s, 1H), 8.14 (s, 1H), 7.98 (s, 1H), 7.82 (d, J = 8.4 Hz, 1H), 7.65 (dd, J = 8.4, 1.2 Hz, 1H), 7.35-7.45 (m, 1H), 7.08-7.19 (m, 3H), 5.40 (s, 2H), 4.55-4.60 (m, 1H), 2.73-2.79 (m, 1H), 2.62-2.68 (m, 1H), 2.54- 2.58 (m, 1H), 2.21-2.33 (m, 1H). LCMS: calc. for C₂₂H₁₇FN₄O₃: 404.1, found: [M + H]⁺ 404.9. |
| 445 | 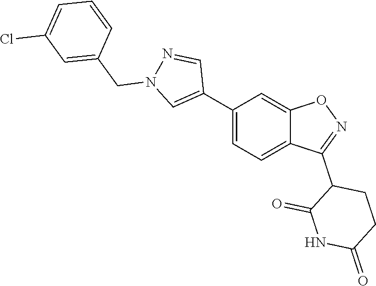 [SMILES: O=C1CCC(c2noc3cc(-c4cnn(Cc5cccc(Cl)c5)c4)ccc23)C(=O)N1] | ¹H NMR (400 MHz, DMSO-d₆) δ 11.08 (s, 1H), 8.51 (s, 1H), 8.13 (s, 1H), 7.97 (s, 1H), 7.81 (d, J = 8.4 Hz, 1H), 7.64 (dd, J = 8.4, 1.2 Hz, 1H), 7.34-7.41 (m, 3H), 7.23-7.30 (m 1H), 5.39 (s, 2H), 4.55-4.60 m, 1H), 2.73-2.80 (m, 1H), 2.61-2.65 (m, 1H), 2.55- 2.59 (m, 1H), 2.18-2.24 (m, 1H). LCMS: calc. for C₂₂H₁₇ClN₄O₃: 420.1, found: [M + H]⁺ 420.9. |
| 446 | 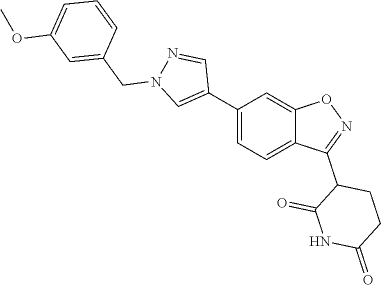 [SMILES: COc1cccc(Cn2cc(-c3ccc4c(C5CCC(=O)NC5=O)noc4c3)cn2)c1] | ¹H NMR (400 MHz, DMSO-d₆) δ 11.07 (s, 1H), 8.48 (s, 1H), 8.11 (s, 1H), 7.97 (s, 1H), 7.80 (d, J = 8.4 Hz, 1H), 7.64 (dd, J = 8.0, 1.2 Hz, 1H), 7.27 (dd, J = 8.8, 7.6 Hz, 1H), 6.81-6.91 (m, 3H), 5.33 (s, 2H), 4.54-4.59 (m, 1H), 3.73 (s, 3H), 2.72-2.80 (m, 1H), 2.63-2.65 (m, 1H), 2.57-2.60 (m, 1H), 2.21- 2.23 (m, 1H). LCMS: calc. for C₂₃H₂₀N₄O₄: 416.2, found: [M + H]⁺ 417.0. |
| 447 | 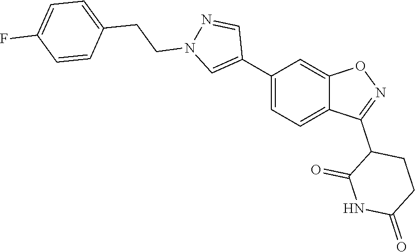 [SMILES: O=C1CCC(c2noc3cc(-c4cnn(CCc5ccc(F)cc5)c4)ccc23)C(=O)N1] | ¹H NMR (400 MHz, DMSO-d₆) δ 11.09 (s, 1H), 8.29 (s, 1H), 8.08 (s, 1H), 7.92 (s, 1H), 7.80 (d, J = 8.4 Hz, 1H), 7.59 (dd, J = 8.4, 1.2 Hz, 1H), 7.19-7.25 (m, 2H), 7.09 (t, J = 8.8 Hz, 2H), 4.54-4.59 (m, 1H), 4.36 (t, J = 7.2 Hz, 2H), 3.15 (t, J = 7.2 Hz, 2H), 2.72- 2.80 (m, 1H), 2.61-2.66 (m, 1H), 2.58-2.60 (m, 1H), 2.20-2.24 (m, 1H). LCMS: calc. for C₂₃H₁₉FN₄O₃: 418.1, found: [M + H]⁺ 419.0. |
| 448 | 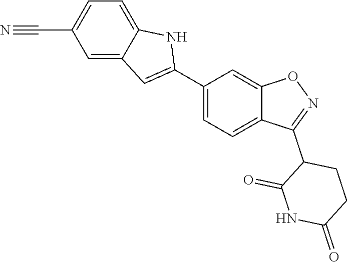 [SMILES: N#Cc1ccc2[nH]c(-c3ccc4c(C5CCC(=O)NC5=O)noc4c3)cc2c1] | ₁H NMR (400 MHz, DMSO-d₆) δ 12.34 (brs, 1H), 11.13 (s, 1H), 8.27 (s, 1H), 8.14 (s, 1H), 7.97 (s, 2H), 7.60 (d, J = 8.4 Hz, 1H), 7.50 (dd, J = 8.4, 1.6 Hz, 1H), 7.31 (s, 1H), 4.60-4.67 (m, 1H), 2.75-2.85 (m, 1H), 2.58-2.68 (m, 2H), 2.20-2.27 (m, 1H). LCMS: calc. for C₂₁H₁₄N₄O₃: 370.1, found: [M + H]⁺ 371.2. |
| 449 | 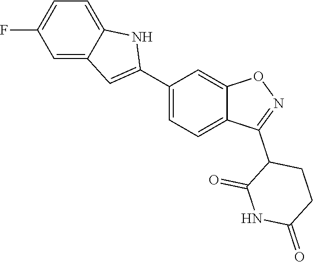 [SMILES: O=C1CCC(c2noc3cc(-c4cc5cc(F)ccc5[nH]4)ccc23)C(=O)N1] | ¹H NMR (400 MHz, DMSO-d₆) δ 11.87 (brs, 1H), 11.14 (brs, 1H), 8.21 (s, 1H), 7.92 (s, 2H), 7.43 (dd, J = 8.8, 4.4 Hz, 1H), 7.29- 7.37 (m, 1H), 7.15 (s, 1H), 6.96-7.04 (m, 1H), 4.58-4.66 (m, 1H), 2.73-2.86 (m, 1H), 2.55-2.69 (m, 2H), 2.16-2.29 (m, 1H). LCMS: calc. for C₂₀H₁₄FN₃O₃: 363.1, found: [M + H]⁺ 364.1. |
| 450 | 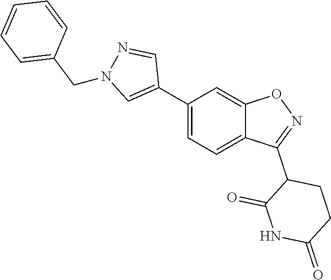 [SMILES: O=C1CCC(c2noc3cc(-c4cnn(Cc5ccccc5)c4)ccc23)C(=O)N1] | ¹H NMR (400 MHz, DMSO-d₆) δ: 11.10 (s, 1H), 8.50 (s, 1H), 8.12 (s, 1H), 7.97 (s, 1H), 7.81 (d, J = 8.4 Hz, 1H), 7.64 (d. J = 8.4 Hz, 1H), 7.29-7.38 (m, 5H), 5.37 (s, 2H), 4.58 (dd, J = 11.6, 4.8 Hz, 1H), 2.73-2.78 (m, 1H), 2.59-2.63 (m, 2H), 2.18-2.23 (m, 1H). LCMS: calc. for C₂₂H₁₈N₄O₃: 386.1, found: [M + H]⁺ 387.1. |
| 451 | 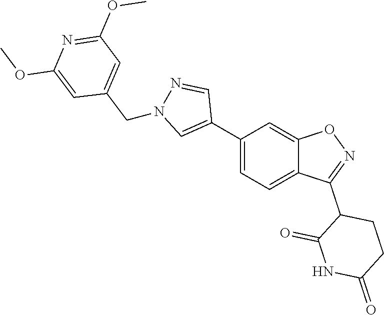 [SMILES: COc1cc(Cn2cc(-c3ccc4c(C5CCC(=O)NC5=O)noc4c3)cn2)cc(OC)n1] | ¹H NMR (400 MHz, DMSO-d₆) δ 11.10 (s, 1H), 8.50 (s, 1H), 8.15 (s, 1H), 7.99 (s, 1H), 7.82 (d, J = 8.4 Hz, 1H), 7.65 (d, J = 8.0 Hz, 1H), 6.19 (s, 2H), 5.33 (s, 2H), 4.58 (dd, J = 12.0, 4.8 Hz, 1H), 3.82 (s, 6H), 2.72-2.81 (m, 1H), 2.51-2.69 (m, 2H), 2.15-2.30 (m, 1 H). LCMS: calc. for C₂₃H₂₁N₅O₅: 447.1, found: [M + H]⁺ 448.3. |
| 452 | 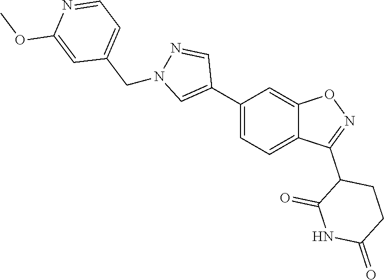 [SMILES: COc1cc(Cn2cc(-c3ccc4c(C5CCC(=O)NC5=O)noc4c3)cn2)ccn1] | ¹H NMR (400 MHz, DMSO-d₆) δ 11.11 (brs, 1H), 8.52 (s, 1H), 8.16 (s, 1H), 8.12 (d, J = 5.2 Hz, 1H), 7.99 (s, 1H), 7.82 (d, J = 8.4 Hz, 1H), 7.65 (d, J = 8.4 Hz, 1H), 6.81 (d, J = 6.4 Hz, 1H), 6.58 (s, 1H), 5.39 (s, 2H), 4.58 (dd, J = 11.6, 5.2 Hz, 1H), 3.82 (s, 3H), 2.71-2.81 (m, 1H), 2.51-2.65 (m, 2H) 2.12-2.25 (m, 1H). LCMS: calc. for C₂₂H₁₉N₅O₄: 417.1, found: [M + H]⁺ 418.1 |
| 453 | 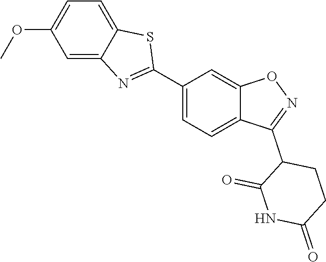 [SMILES: COc1ccc2sc(-c3ccc4c(C5CCC(=O)NC5=O)noc4c3)nc2c1] | ¹H NMR (400 MHz, DMSO-d₆) δ 11.17 (brs, 1H), 8.41 (s, 1H), 8.08-8.13 (m, 1H), 7.99-8.07 (m, 2H), 7.79 (d, J = 2.4 Hz, 1H), 7.19 (dd, J = 8.8, 2.4 Hz, 1H), 4.65-4.75 (m, 1H), 3.88 (s, 3H), 2.75-2.85 (m, 1H), 2.52- 2.65 (m, 2H), 2.21-2.31 (m, 1H). LCMS: calc. for C₂₁H₁₇N₃O₄: 393.1, found: [M + H]⁺ 394.1. |
| 454 | 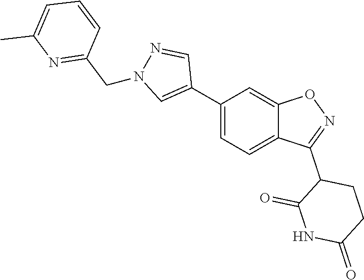 [SMILES: Cc1cccc(Cn2cc(-c3ccc4c(C5CCC(=O)NC5=O)noc4c3)cn2)n1] | ¹H NMR (400 MHz, DMSO-d₆) δ 11.11 (brs, 1H), 8.52 (s, 1H), 8.15 (s, 1H), 8.00 (s, 1H), 7.82 (d, J = 8.4 Hz, 1H), 7.64-7.69 (m, 2H), 7.19 (d, J = 7.6 Hz, 1H), 6.89 (d, J = 7.6 Hz, 1H), 5.42 (s, 2H), 4.55-4.61 (m, 1H), 2.71-2.81 (m, 1H), 2.57-2.69 (m, 2H), 2.46 (s, 3H), 2.17-2.23 (m, 1H). LCMS: calc. for C₂₂H₁₉N₅O₃: 401.1, found: [M + H]⁺ 402.2. |
| 455 | 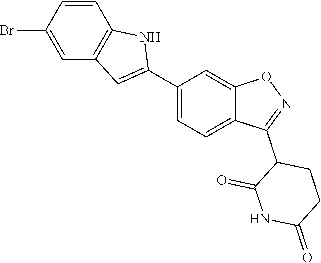 [SMILES: O=C1CCC(c2noc3cc(-c4cc5cc(Br)ccc5[nH]4)ccc23)C(=O)N1] | ¹H NMR (400 MHz, DMSO-d₆) δ 11.97 (brs, 1H), 11.14 (brs, 1H), 8.22 (s, 1H), 7.90-7.96 (m, 2H), 7.77 (s, 1H), 7.41 (d, J = 8.8 Hz, 1H), 7.26 (d, J = 8.4 Hz, 1H), 7.15 (s, 1H), 4.60-4.66 (m, 1H), 2.74-2.85 (m, 1H), 2.57-2.68 (m, 2H), 2.20-2.27 (m, 1H). LCMS: calc. for C₂₀H₁₄BrN₃O₃: 423.0, found: [M + H]⁺ 424.0 |
| 456 | 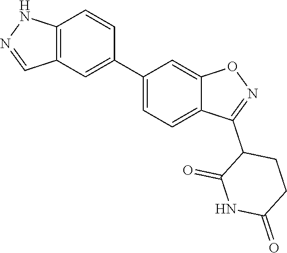 [SMILES: O=C1CCC(c2noc3cc(-c4ccc5[nH]ncc5c4)ccc23)C(=O)N1] | ¹H NMR (400 MHz, DMSO-d₆) δ: 13.20 (s, 1H), 11.13 (s, 1H), 8.18 (d, J = 8.0 Hz, 2H), 8.06 (s, 1H), 7.90-7.97 (m, 1H), 7.70-7.85 (m, 2H), 7.67 (d, J = 8.0 Hz, 1H), 4.58 (dd, J = 11.6, 4.8 Hz, 1H), 2.73-2.78 (m, 1H), 2.59-2.63 (m, 2H), 2.18-2.23 (m, 1H). LCMS: calc. for C₁₉H₁₄N₄O₃: 346.1, found: [M + H]⁺ 347.1. |
| 457 | 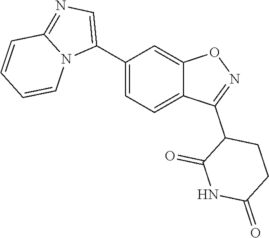 [SMILES: O=C1CCC(c2noc3cc(-c4cnc5ccccn45)ccc23)C(=O)N1] | ¹H NMR (400 MHz, DMSO-d₆) δ: 11.14 (s, 1H), 8.75 (d, J = 6.8 Hz, 1H), 8.10 (s, 1H), 8.01 (d, J = 8.4 Hz, 1H), 7.95 (s, 1H), 7.71 (d, J = 8.4 Hz, 2H), 7.37 (t, J = 8.8 Hz, 1H), 7.02 (t, J = 6.8 Hz, 1H), 4.66 (dd, J = 11.6, 4.8 Hz, 1H), 2.73-2.84 (m, 1H), 2.55-2.63 (m, 2H), 2.18-2.23 (m, 1H). LCMS: calc. for C₁₉H₁₄N₄O₃: 346.1, found: [M + H]⁺ 347.2. |
| 458 | 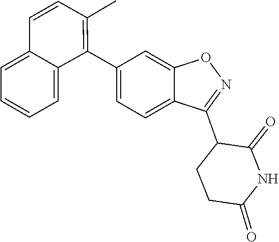 [SMILES: Cc1ccc2ccccc2c1-c1ccc2c(C3CCC(=O)NC3=O)noc2c1] | ¹H NMR (400 MHz, DMSO-d₆) δ 11.16 (brs, 1H), 7.91-8.04 (m, 3H), 7.71 (s, 1H), 7.44-7.53 (m, 2H), 7.38 (t, J = 8.0 Hz, 1H), 7.28 (d, J = 8.4 Hz, 1H), 7.21 (d, J = 8.4 Hz, 1H), 4.65-4.74 (m, 1H), 2.75-2.91 (m, 1H), 2.63-2.74 (m, 2H), 2.27-2.33 (m, 1H), 2.21 (s, 3H). LCMS: calc. for C₂₃H₁₈N₂O₃: 370.1, found: [M + H]⁺ 371.2. |
| 459 | 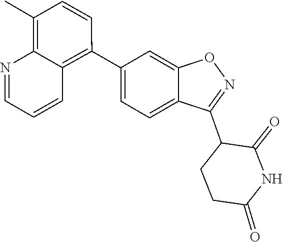 [SMILES: Cc1ccc(-c2ccc3c(C4CCC(=O)NC4=O)noc3c2)c2cccnc12] | ¹H NMR (400 MHz, DMSO-d₆) δ 11.15 (brs, 1H), 8.98 (d, J = 2.8 Hz, 1H), 8.20 (dd, J = 8.8, 1.2 Hz, 1H), 8.00 (d, J = 8.0 Hz, 1H), 7.87 (s, 1H), 7.73 (d, J = 7.6 Hz, 1H), 7.47-7.57 (m, 3H), 4.65-4.74 (m, 1H), 2.75- 2.83 (m, 4H), 2.57-2.68 (m, 2H), 2.21-2.31 (m, 1H). LCMS: calc. for C₂₂H₁₇N₃O₃: 371.1, found: [M + H]⁺ 372.2 |
| 460 | 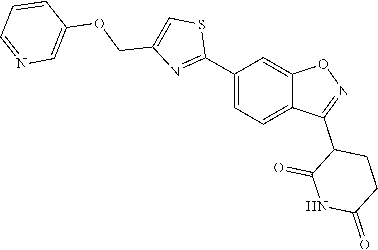 [SMILES: O=C1CCC(c2noc3cc(-c4nc(COc5cccnc5)cs4)ccc23)C(=O)N1] | ¹H NMR (400 MHz, DMSO-d₆) δ 11.13 (brs, 1H), 8.42 (d, J = 2.8 Hz, 1H), 8.31 (s, 1H), 8.20 (d, J = 8.8 Hz, 1H), 7.92-8.02 (m, 3H), 7.56 (d, J = 7.6 Hz, 1H), 7.32-7.40 (m, 1H), 5.35 (s, 2H), 4.60-4.70 (m, 1H), 2.70- 2.80 (m, 1H), 2.55-2.65 (m, 2H), 2.14-2.24 (m, 1H). LCMS: calc. for C₂₁H₁₆N₄O₄S: 420.1, found: [M + H]⁺ 421.1 |
| 461 | 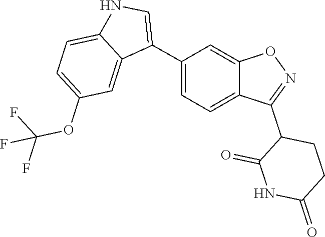 [SMILES: O=C1CCC(c2noc3cc(-c4c[nH]c5ccc(OC(F)(F)F)cc45)ccc23)C(=O)N1] | ¹H NMR (400 MHz, DMSO-d₆) δ 11.88 (brs, 1H), 11.13 (brs, 1H), 8.08 (d, J = 2.4 Hz, 1H), 7.99 (s, 1H), 7.85-7.92 (m, 2H), 7.73 (dd, J = 8.4, 1.2 Hz, 1H), 7.58 (d, J = 8.8 Hz, 1H), 7.19 (d, J = 8.8 Hz, 1H), 4.55- 4.68 (m, 1H), 2.73-2.85 (m, 1H), 2.52-2.63 (m, 2H), 2.18-2.27 (m, 1H). LCMS: calc. for C₂₁H₁₄F₃N₃O₄: 429.1, found: [M + H]⁺ 430.1. |
| 462 | 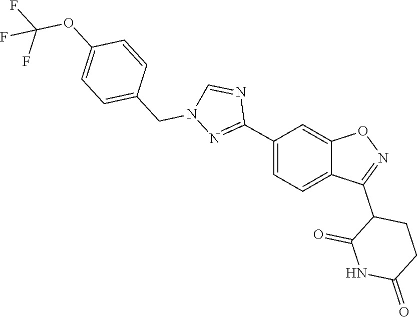 [SMILES: O=C1CCC(c2noc3cc(-c4ncn(Cc5ccc(OC(F)(F)F)cc5)n4)ccc23)C(=O)N1] | ¹H NMR (400 MHz, DMSO-d₆) δ 11.12 (brs, 1H), 8.85 (s, 1H), 8.23 (s, 1H), 8.00- 8.04 (m, 1H), 7.94 (d, J = 8.4 Hz, 1H), 7.49- 7.52 (m, 2H), 7.39 (d, J = 8.0 Hz, 2H), 5.56 (s, 2H), 4.61-4.67 (m, 1H), 2.75-2.81 (m, 1H), 2.58-2.61 (m, 2H), 2.22-2.27 (m, 1H). LCMS: calc. for C₂₂H₁₆F₃N₅O₄: 471.1, found: [M + H]⁺ 472.0. |
| 463 | 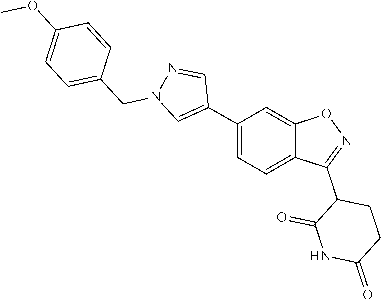 [SMILES: COc1ccc(Cn2cc(-c3ccc4c(C5CCC(=O)NC5=O)noc4c3)cn2)cc1] | ¹H NMR (400 MHz, DMSO-d₆) δ 11.09 (s, 1H), 8.44 (s, 1H), 8.08 (s, 1H), 7.95 (s, 1H), 7.81-7.79 (m, 1H), 7.64-7.62 (m, 1H), 7.27 (d, J = 8.4 Hz, 2H), 6.92 (d, J = 8.8 Hz, 2H), 5.28 (s, 2H), 4.59-4.55 (m, 1H), 3.73 (s, 3H), 2.77-2.67 (m, 1H), 2.63-2.62 (m, 1H), 2.59-2.56 (m, 1H), 2.23-2.22 (m, 1H). MS: (ESI) m/z [M + H]⁺ 417.3 |
| 464 | 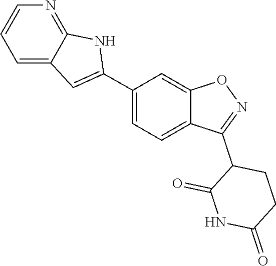 [SMILES: O=C1CCC(c2noc3cc(-c4cc5cccnc5[nH]4)ccc23)C(=O)N1] | ¹H NMR (400 MHz, DMSO-d₆) δ 11.99 (s, 1H), 11.15 (s, 1H), 8.32-8.36 (m, 2H), 8.25 (s, 1H), 7.94-8.02 (m, 2H), 7.81 (d, J = 8.0 Hz, 1H), 7.32 (s, 1H), 7.15 (dd, J = 8.0, 4.4 Hz, 1H), 4.64 (dd, J = 12.0, 4.8 Hz, 1H), 2.76-2.82 (m, 1H), 2.61 (s, 1H), 2.20-2.27 (m, 1H), 1.95-2.04 (m, 1H). LCMS: calc. for C₁₉H₁₄N₄O₃: 346.1, found: [M + H]⁺ 347.0. |
| 465 | 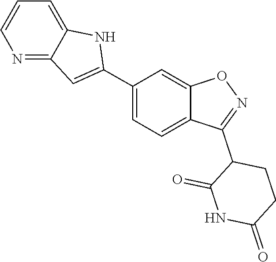 [SMILES: O=C1CCC(c2noc3cc(-c4cc5ncccc5[nH]4)ccc23)C(=O)N1] | ¹H NMR (400 MHz, DMSO-d₆) δ 13.51 (s, 1H), 11.12 (s, 1H), 8.62-8.67 (m, 1H), 8.56 (s, 1H), 8.52 (d, J = 7.9 Hz, 1H), 8.13-8.18 (m, 1H), 8.07-8.11 (m, 1H), 7.64 (dd, J = 8.0, 5.6 Hz, 1H), 7.56 (s, 1H), 4.69 (dd, J = 12.0, 4.8 Hz, 1H), 2.76-2.82 (m, 1H), 2.54- 2.60 (m, 2H), 2.16-2.30 (m, 1H). LCMS: calc. for C₁₉H₁₄N₄O₃: 346.1, found: [M + H]⁺ 346.9. |
| 466 | 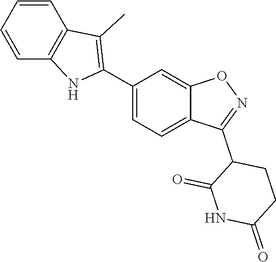 [SMILES: Cc1c(-c2ccc3c(C4CCC(=O)NC4=O)noc3c2)[nH]c2ccccc12] | ¹H NMR (400 MHz, DMSO-d₆) δ 11.37 (brs, 1H), 11.15 (brs, 1H), 7.94-8.00 (m, 2H), 7.72 (d, J = 8.8 Hz, 1H), 7.59 (d, J = 8.0 Hz, 1H), 7.40 (d, J = 8.0 Hz, 1H), 7.13- 7.18 (m, 1H), 7.02-7.07 (m, 1H), 4.61-4.67 (m, 1H), 2.75-2.85 (m, 1H), 2.52-2.63 (m, 2H), 2.49 (s, 3H), 2.20-2.28 (m, 1H). LCMS: calc. for C₂₁H₁₇N₃O₃: 359.1, found: [M + H]⁺ 360.1. |
| 467 | 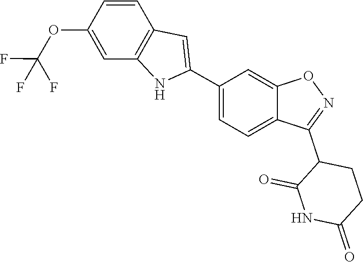 [SMILES: O=C1CCC(c2noc3cc(-c4cc5ccc(OC(F)(F)F)cc5[nH]4)ccc23)C(=O)N1] | ¹H NMR (400 MHz, DMSO-d₆) δ 12.01 (brs, 1H), 11.11 (brs, 1H), 8.21 (s, 1H), 7.90-7.98 (m, 2H), 7.68 (d, J = 8.8 Hz, 1H), 7.36 (s, 1H), 7.23 (d, J = 1.6 Hz, 1H), 6.98- 7.05 (m, 1H), 4.60-4.65 (m, 1H), 2.76-2.83 (m, 1H), 2.57-2.63 (m, 2H), 2.21-2.26 (m, 1H). LCMS: calc. for C₂₁H₁₄F₃N₃O₄: 429.1, found: [M + H]⁺ 430.1. |
| 468 | 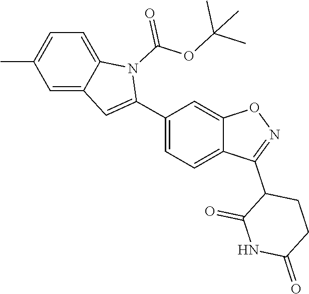 [SMILES: Cc1ccc2c(c1)cc(-c1ccc3c(C4CCC(=O)NC4=O)noc3c1)n2C(=O)OC(C)(C)C] | ¹H NMR (400 MHz, DMSO-d₆) δ 11.13 (brs, 1H), 7.99 (d, J = 8.4 Hz, 1H), 7.86- 7.91 (m, 2H), 7.42-7.50 (m, 2H), 7.19 (d, J = 8.0 Hz, 1H), 6.81 (s, 1H), 4.61-4.68 (m, 1H), 2.75-2.86 (m, 1H), 2.54-2.68 (m, 2H), 2.41 (s, 3H), 2.19-2.28 (m, 1H), 1.25 (s, 9H). LCMS: calc. for C₂₆H₂₅N₃O₅: 459.2, found: [M + H]⁺ 460.2. |
| 469 | 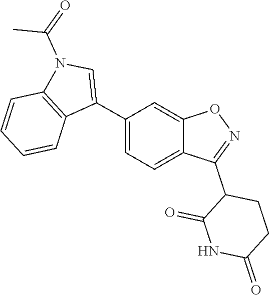 [SMILES: CC(=O)n1cc(-c2ccc3c(C4CCC(=O)NC4=O)noc3c2)c2ccccc21] | ¹H NMR (400 MHz, DMSO-d₆) δ 11.14 (brs, 1H), 8.46 (d, J = 7.6 Hz, 1H), 8.35 (s, 1H), 8.12 (s, 1H), 7.98 (d, J = 8.4 Hz, 2H), 7.79-7.85 (m, 1H), 7.36-7.49 (m, 2H), 4.60- 4.72 (m, 1H), 2.72-2.85 (m, 4H), 2.54-2.65 (m, 2H), 2.20-2.28 (m, 1H). LCMS: calc. for C₂₂H₁₇N₃O₄: 387.1, found: [M + H]⁺ 388.1. |
| 470 | 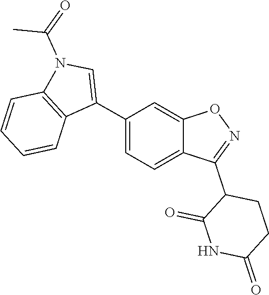 [SMILES: CC(=O)n1cc(-c2ccc3c(C4CCC(=O)NC4=O)noc3c2)c2ccccc21] | ¹H NMR (400 MHz, DMSO-d₆) δ 11.12 (s, 1H), 8.47 (d, J = 8.0 Hz, 1H), 8.40 (s, 0.1H, FA), 8.35 (s, 1H), 8.12 (s, 1H), 7.98 (d, J = 8.8, 2H), 7.82 (d, J = 8.8, 1H), 7.33-7.50 (m, 2H), 4.58-4.72 (m, 1H), 2.76 (s, 3H), 2.67 (s, 1H), 2.58-2.65 (m, 1H), 2.54-2.57 (m, 1H), 2.22-2.30 (m, 1H). LCMS: calc. for C₂₂H₁₇N₃O₄: 387.1, found: [M + H]⁺ 388.1. |
| 471 | 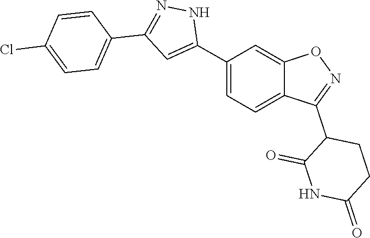 [SMILES: O=C1CCC(c2noc3cc(-c4cc(-c5ccc(Cl)cc5)n[nH]4)ccc23)C(=O)N1] | ¹H NMR (400 MHz, DMSO-d₆) δ 13.57- 13.73 (m, 1H), 11.13 (s, 1H), 8.18 (s, 1H), 7.81-7.98 (m, 4H), 7.49-7.62 (m, 2H), 7.46 (s, 1H), 4.56-4.68 (m, 1H), 2.74-2.84 (m, 1H), 2.61-2.68 (m, 1H), 2.52-2.58 (m, 1H), 2.21-2.28 (m, 1H). LCMS: calc. for C₂₁H₁₅ClN₄O₃: 406.1, found: [M + H]⁺ 406.8. |
| 472 | 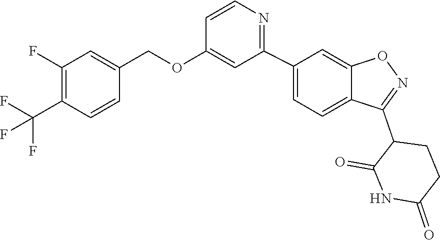 [SMILES: O=C1CCC(c2noc3cc(-c4cc(OCc5ccc(C(F)(F)F)c(F)c5)ccn4)ccc23)C(=O)N1] | ¹H NMR (400 MHz, DMSO-d₆) δ 11.13 (s, 1H), 8.57 (d, J = 5.6 Hz, 1H), 8.46 (s, 1H), 8.19 (dd, J = 8.4, 1.2 Hz, 1H), 7.96 (d, J = 8.4 Hz, 1H), 7.82-7.90 (m, 2H), 7.67 (d, J = 11.6 Hz, 1H), 7.55 (d, J = 8.0 Hz, 1H), 7.13 (dd, J = 5.6, 2.4 Hz, 1H), 5.46 (s, 2H), 4.65 (dd, J = 12.0, 5.2 Hz, 1H), 2.74-2.84 (m, 1H), 2.57-2.67 (m, 2H), 2.19-2.28 (m, 1H). LCMS: calc. for C₂₅H₁₇F₄N₃O₄: 499.4, found: [M + H]⁺ 500.0. |
| 473 | 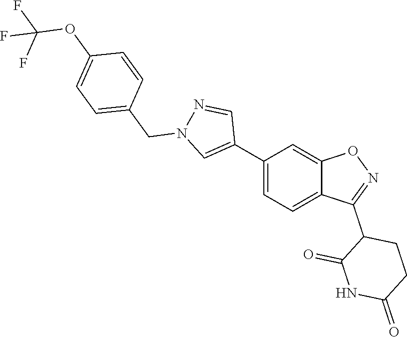 [SMILES: O=C1CCC(c2noc3cc(-c4cnn(Cc5ccc(OC(F)(F)F)cc5)c4)ccc23)C(=O)N1] | ¹H NMR (400 MHz, DMSO-d₆) δ: 11.09 (s, 1H), 8.52 (s, 1H), 8.14 (s, 1H), 7.97 (s, 1H), 7.80-7.83 (m, 1H), 7.63-7.67 (m, 1H), 7.35- 7.45 (m, 4H), 5.42 (s, 2H), 4.58 (dd, J = 11.6, 4.8 Hz, 1H), 2.73-2.81 (m, 1H), 2.55- 2.63 (m, 2H), 2.18-2.23 (m, 1H). LCMS: calc. for C₂₃H₁₇N₄O₄: 470.1, found: [M + H]⁺ 471.1. |
| 474 | 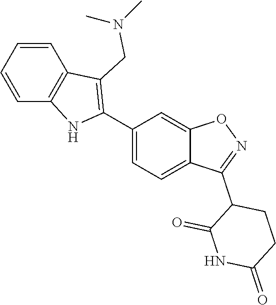 [SMILES: CN(C)Cc1c(-c2ccc3c(C4CCC(=O)NC4=O)noc3c2)[nH]c2ccccc12] | ¹H NMR (400 MHz, DMSO-d₆) δ 11.65 (brs, 1H), 11.15 (brs, 1H), 8.35 (s, 1H), 8.18 (s, 1H), 7.93-8.00 (m, 2H), 7.70 (d, J = 8.0 Hz, 1H), 7.43 (d, J = 8.0 Hz, 1H), 7.17 (t, J = 7.2 Hz, 1H), 7.04-7.09 (m, 1H), 4.61-4.67 (m, 1H), 3.59 (s, 2H), 2.76-2.84 (m, 1H), 2.52-2.63 (m, 2H), 2.25-2.30 (m, 7H). LCMS: calc. for C₂₃H₂₂N₄O₃: 402.2, found: [M + H]⁺ 403.2. |
| 475 | 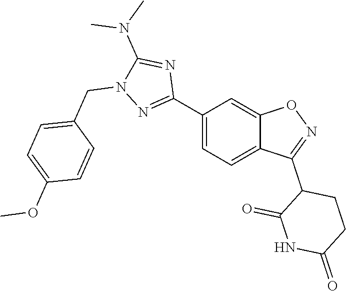 [SMILES: COc1ccc(Cn2nc(-c3ccc4c(C5CCC(=O)NC5=O)noc4c3)nc2N(C)C)cc1] | ¹H NMR (400 MHz, DMSO-d₆) δ 11.11 (brs, 1H), 8.14 (s, 1H), 7.98 (d, J = 8.0 Hz,, 1H), 7.90 (d, J = 8.4 Hz, 1H), 7.22 (d, J = 8.8 Hz, 2H), 6.93 (d, J = 8.8 Hz, 2H), 5.29 (s, 2H), 4.60-4.66 (m, 1H), 3.73 (s, 3H), 2.90 (s, 6H), 2.73-2.81 (m, 1H), 2.60-2.66 (m, 2H), 2.21-2.26 (m, 1H). LCMS: calc. for C₂₄H₂₄N₆O₄: 460.2, found: [M + H]⁺ 461.2. |
| 476 | 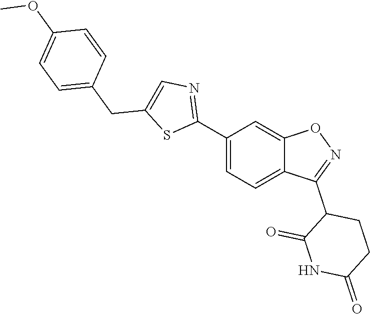 [SMILES: COc1ccc(Cc2cnc(-c3ccc4c(C5CCC(=O)NC5=O)noc4c3)s2)cc1] | ¹H NMR (400 MHz, DMSO-d₆) δ: 11.13 (s, 1H), 8.25 (s, 1H), 7.95 (s, 2H), 7.40 (s, 1H), 7.26 (d, J = 8.8 Hz 2H), 6.89 (d, J = 8.8 Hz 2H), 4.65 (dd, J = 11.6, 4.8 Hz, 1H), 4.09 (s, 2H), 3.72 (s, 3H), 2.73-2.81 (m, 1H), 2.55- 2.63 (m, 2H), 2.18-2.23 (m, 1H). LCMS: calc. for C₂₃H₁₉N₃O₄S: 433.1, found: [M + H]⁺ 434.2. |
| 477 | 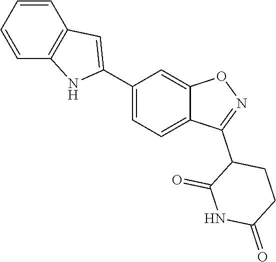 [SMILES: O=C1CCC(c2noc3cc(-c4cc5ccccc5[nH]4)ccc23)C(=O)N1] | ¹H NMR (400 MHz, DMSO-d₆) δ 11.73 (s, 1H), 11.13 (s, 1H), 8.21 (s, 1H), 7.92 (s, 2H), 7.58 (d, J = 8.0 Hz, 1H), 7.44 (d, J = 8.0 Hz, 1H), 7.10-7.19 (m, 2H), 6.97-7.06 (m, 1H), 4.62 (dd, J = 11.6, 4.8 Hz, 1H), 2.73-2.85 (m, 1H), 2.53-2.68 (m, 2H), 2.17- 2.27 (m, 1H). LCMS: calc. for C₂₀H₁₅N₃O₃: 345.1, found: [M + H]⁺ 346.1. |
| 478 | 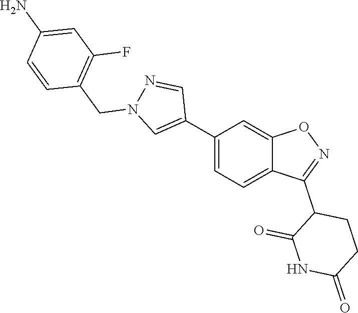 [SMILES: Nc1ccc(Cn2cc(-c3ccc4c(C5CCC(=O)NC5=O)noc4c3)cn2)c(F)c1] | ¹H NMR (400 MHz, DMSO-d₆) δ 11.03- 11.12 (s, 1H), 8.47 (s, 0.6H, FA), 8.33 (s, 1H), 8.06 (s, 1H), 7.97 (s, 1H), 7.78-7.81 (m, 1H), 7.62-7.65 (m, 1H), 7.02-7.07 (m, 1H), 6.28-6.44(m, 2H), 5.49 (s, 2H), 5.18 (s, 2H), 4.51-4.60 (m, 1H), 2.73-2.79 (m, 1H), 2.50-2.65 (m, 1H), 2.13-2.38 (m, 2H). LCMS: calc. for C₂₂H₁₈FN₅O₃: 419.1, found: [M + H]⁺ 420.0 |
| 479 | 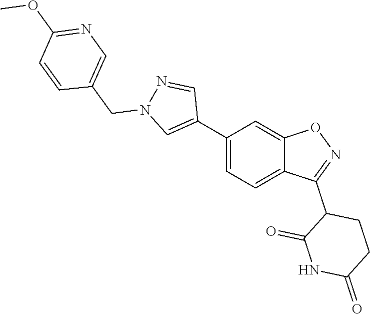 [SMILES: COc1ccc(Cn2cc(-c3ccc4c(C5CCC(=O)NC5=O)noc4c3)cn2)cn1] | ¹H NMR (400 MHz ,DMSO-d₆) δ 10.93- 11.23 (m, 1H), 8.46-8.52 (m, 1H), 8.22 (brs, 1H), 8.11 (s, 1H), 7.94-8.00 (m, 1H), 7.81 (d, J = 8.0 Hz, 1H), 7.62-7.72 (m, 2H), 6.83 (d, J = 8.0 Hz, 1H), 5.32 (s, 2H), 4.58 (d, J = 12.0, 4.0 Hz, 1H), 3.84 (s, 3H), 2.72-2.81 (m, 1H), 2.62 (d, J = 16.0 Hz, 2H), 2.18- 2.26 (m, 1H). LCMS: calc. for C₂₂H₁₉N₅O₄: 417.1, found: [M + H]⁺ 418.0. |
| 480 | 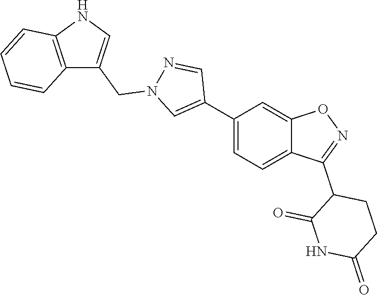 [SMILES: O=C1CCC(c2noc3cc(-c4cnn(Cc5c[nH]c6ccccc56)c4)ccc23)C(=O)N1] | ¹H NMR (400 MHz, DMSO-d₆) δ 13.06- 13.13 (m, 1H), 11.13 (s, 1H), 8.38 (s, 1H), 8.05 (s, 1H), 7.92 (s, 1H), 7.76 (d, J = 8.4 Hz, 1H), 7.57-7.61 (m, 2H), 7.49 (d, J = 2.4 Hz, 1H), 7.38 (d, J = 8.0 Hz, 1H), 7.06-7.11 (m, 1H), 6.96-7.01 (m, 1H), 5.48 (s, 2H), 4.53-4.58 (m, 1H), 2.74-2.80 (m, 1H), 2.61- 2.64 (m, 1H), 2.56-2.60 (m, 1H), 2.17-2.22 (m, 1H). LCMS: calc. for C₂₄H₁₉N₅O₃: 425.1, found: [M + H]⁺ 425.9. |

### Example 119:3-[5-fluoro-6-[1-[(4-methylsulfanylphenyl)methyl]pyrazol-4-yl]-1,2-benzoxazol-3-yl]piperidine-2,6-dione

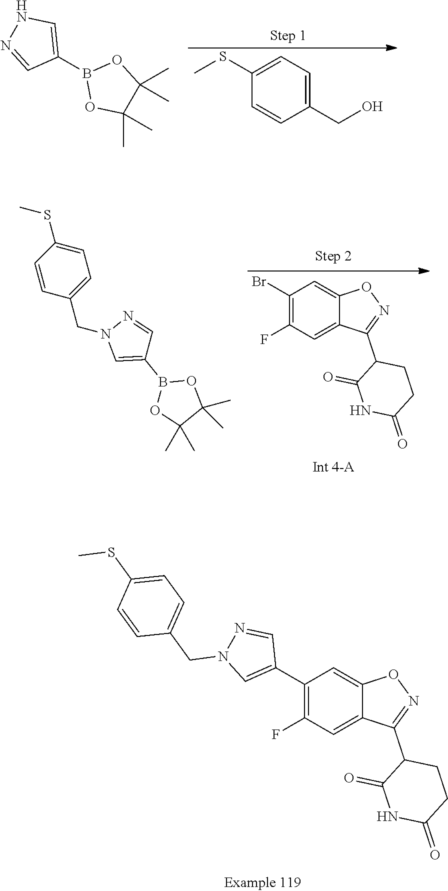

Step 1: To a solution of (4-methylsulfanylphenyl) methanol (1 g, 6.48 mmol, 1 eq), 4-(4,4,5,5-tetramethyl-1,3,2-dioxaborolan-2-yl)-1H-pyrazole (1.38 g, 7.13 mmol, 1.1 eq) and PPh3 (2.04 g, 7.78 mmol, 1.2 eq) in THF (20 mL) was added DIAD (1.57 g, 7.78 mmol, 1.2 eq) at 0° C. The mixture was stirred at 20° C. for 2 h. The reaction mixture was concentrated. Water (100 mL) was added to the residue. The resulting mixture was extracted with EtOAc (60 mL×3). The combined organic phase was washed with water (200 mL), dried over anhydrous Na₂SO₄, filtered and concentrated. The residue was purified by flash silica gel chromatography to give 1-[(4-methylsulfanylphenyl)methyl]-4-(4,4,5,5-tetramethyl-1,3,2-dioxaborolan-2-yl)pyrazole (2.5 g, 77.83% yield) as a yellow oil. LCMS: calc. for C₁₇H₂₃BN₂O₂S: 329.1, found: [M+H]⁺330.8. ¹H NMR (400 MHZ, CDCl₃) δ 7.80 (s, 1H), 7.64 (s, 1H), 7.19-7.22 (m, 2H), 7.13-7.17 (m, 2H), 5.25 (s, 2H), 2.45 (s, 3H), 1.29 (s, 12H).

Step 2: To a solution of Int 4-A (30 mg, 91.71 μmol, 1 eq) in dioxane (2 mL) and H₂O (0.5 mL) was added 1-[(4-methylsulfanylphenyl)methyl]-4-(4,4,5,5-tetramethyl-1,3,2-dioxaborolan-2-yl)pyrazole (36.35 mg, 110.06 μmol, 1.2 eq), XPhos Pd G3 (7.76 mg, 9.17 μmol, 0.1 eq) and K₃PO₄ (29.20 mg, 137.57 μmol, 1.5 eq) at 20° C. The mixture was stirred at 80° C. for 1 h under N₂. The reaction mixture was concentrated directly. The residue was purified by prep-HPLC to give Example 119 (1.4 mg, 3.39% yield) as a white solid. LCMS: calc. for C₂₃H₁₉FN₄₀₃S: 450.1, found: [M+H]⁺451.2. ¹H NMR (400 MHZ, DMSO-d₆) δ 11.10 (br d, J=4.0 Hz, 1H), 8.43 (d, J=2.8 Hz, 1H), 8.18-8.11 (m, 2H), 7.84 (d, J=8.0 Hz, 1H), 7.29-7.23 (m, 4H), 5.38-5.33 (m, 2H), 4.57 (dd, J=4.8, 12.0 Hz, 1H), 2.82-2.71 (m, 1H), 2.67 (s, 2H), 2.45 (s, 3H), 2.23-2.14 (m, 1H).

### Example 120:3-[5-fluoro-6-[1-[[4-(methoxymethyl)phenyl]methyl]pyrazol-4-yl]-1,2-benzoxazol-3-yl]piperidine-2,6-dione

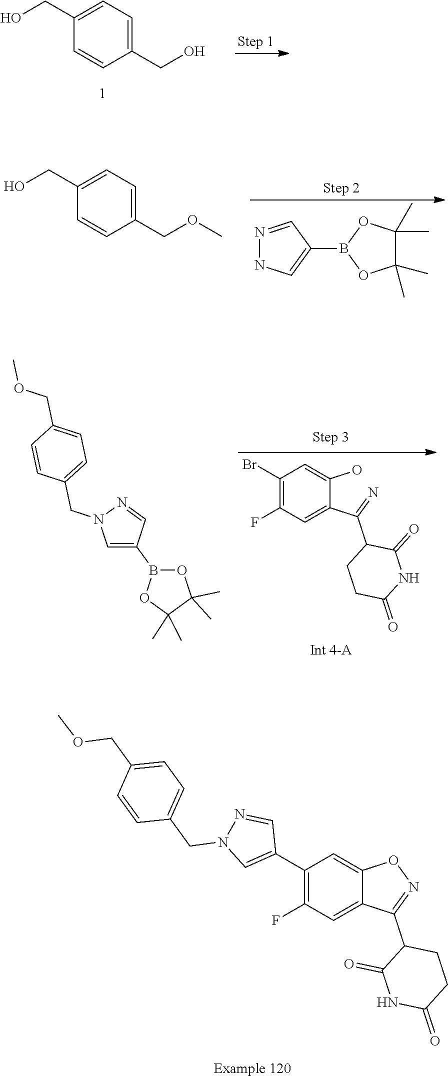

Step 1: To a solution of [4-(hydroxymethyl)phenyl]methanol (1.0 g, 7.24 mmol, 1 eq) in THF (15 mL) was added Cs₂CO₃ (9.43 g, 28.95 mmol, 4 eq), the mixture was stirred at 25° C. for 0.5 h, then added Mel (1.03 g, 7.24 mmol, 1 eq). The mixture was stirred at 25° C. for 16 h. The mixture was diluted with H₂O (50 mL), then was extracted with EtOAc (30 mL×2). The organic layer was washed with brine (50 mL×2), dried over Na₂SO₄, filtered and concentrated under reduced pressure to give a residue. The residue was purified by flash silica gel chromatography. Product [4-(methoxymethyl)phenyl]methanol (290 mg, 26.33% yield) was obtained as a colorless oil. ¹H NMR (400 MHZ, DMSO-d₆) δ 7.31-7.24 (m, 4H), 4.48 (d, J=5.6 Hz, 2H), 4.38 (s, 2H), 3.27 (s, 3H).

Step 2: A mixture of [4-(methoxymethyl)phenyl]methanol (100 mg, 0.657 mmol, 1 eq) and 4-(4,4,5,5-tetramethyl-1,3,2-dioxaborolan-2-yl)-1H-pyrazole (127.50 mg, 0.657 mmol, 1 eq) in Toluene (3 mL) was stirred for 10 min, then tributylphosphane (199.41 mg, 0.986 mmol, 1.5 eq) and TMAD (169.71 mg, 0.986 mmol, 1.5 eq) was added, the reaction mixture was stirred at 25° C. for 16 hr. The mixture was diluted with H₂O (20 mL), then was extracted with EtOAc (15 mL×2). The organic layer was washed with brine (20 mL×2), dried over Na₂SO₄, filtered and concentrated under reduced pressure to give a residue. Product 1-[[4-(methoxymethyl)phenyl]methyl]-4-(4,4,5,5-tetramethyl-1,3,2-dioxaborolan-2-yl)pyrazole (140 mg, 51.93% yield) was obtained as a colorless oil. LCMS: calc. for C₁₈H₂₅BN₂O₃: 328.2, found: [M+H]⁺328.9.

Step 3: To a mixture of Int 4-A (51.03 mg, 156.00 μmol, 0.8 eq) in dioxane (2 mL) and H₂O (0.5 mL) was added 1-[[4-(methoxymethyl)phenyl]methyl]-4-(4,4,5,5-tetramethyl-1,3,2-dioxaborolan-2-yl)pyrazole (80 mg, 195.00 μmol, 80% purity, 1 eq), K₃PO₄ (62.09 mg, 292.49 μmol, 1.5 eq) and Xphos Pd G3 (16.51 mg, 19.50 μmol, 0.1 eq). The mixture was degassed and purged with N₂ for 3 times, and then was stirred at 80° C. for 1.5 hr under N₂ atmosphere. The mixture was diluted with H₂O (20 mL), then was extracted with EtOAc (15 mL×2). The organic layer was washed with brine (20 mL×2), dried over Na₂SO₄, filtered and concentrated under reduced pressure to give a residue. The was residue purified by prep-HPLC, and dried by lyophilization to afford Example 120 (5.6 mg, 6.26% yield) as a white solid. LCMS: calc. for C₂₄H₂₁₀₄N₄F: 448.2, found: [M+H]⁺449.1. ¹H NMR (400 MHZ, DMSO-d₆)_{8=11.12}-11.06 (m, 1H), 8.44 (br s, 1H), 8.18-8.09 (m, 2H), 7.83 (br d, J=10.0 Hz, 1H), 7.30 (s, 4H), 5.42-5.39 (m, 1H), 5.40 (s, 1H), 4.60-4.53 (m, 1H), 4.38 (s, 2H), 3.27 (br s, 3H), 2.81-2.71 (m, 1H), 2.65 (br d, J=15.6 Hz, 1H), 2.33 (br d, J=1.2 Hz, 1H), 2.23-2.15 (m, 1H).

Examples 121-125 and 481-497 were prepared following the similar procedure that was used for preparing Examples 119-120, starting from Int 4-A and their corresponding boronic acid or ester intermediates, with modifications. Characterizations of Examples 121-125 and 481-497 are provided in Table 4.

TABLE 4

| Example | | |
|---|---|---|
| ID | Structure | NMR/MS |
| 121 | 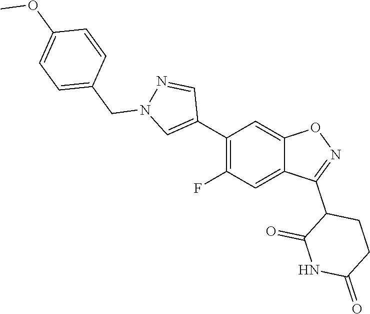 [SMILES: COc1ccc(Cn2cc(-c3cc4onc(C5CCC(=O)NC5=O)c4cc3F)cn2)cc1] | ¹H NMR (400 MHz, d₆-DMSO) δ 11.10 (br s, 1H), 8.39 (d, J = 2.8 Hz, 1H), 8.15 (d, J = 5.6 Hz, 1H), 8.11 (s, 1H), 7.83 (d, J = 10.4 Hz, 1H), 7.29 (d, J = 8.4 Hz, 2H), 6.92 (d, J = 8.8 Hz, 2H), 5.32 (s, 2H), 4.57 (dd, J = 4.8, 12.0 Hz, 1H), 3.73 (s, 3H), 2.82-2.71 (m, 1H), 2.64-2.52 (m, 2H), 2.24- 2.13 (m, 1H).LCMS: calc. for C₂₃H₁₉FN₄O₄: 434.1, found: [M + H]⁺ 435.0. |
| 122 | 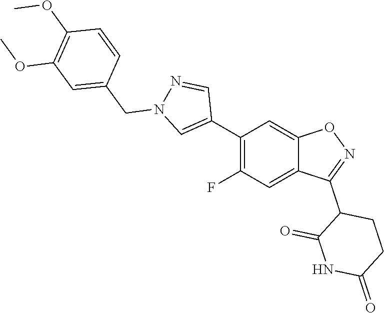 [SMILES: COc1ccc(Cn2cc(-c3cc4onc(C5CCC(=O)NC5=O)c4cc3F)cn2)cc1OC] | ¹H NMR (400 MHz, DMSO-d₆) δ 11.09 (s, 1H), 8.41-8.37 (m, 1H), 8.14 (d, J = 5.6 Hz, 1H), 8.10 (s, 1H), 7.82 (d, J = 10.4 Hz, 1H), 7.01 (d, J = 2.0 Hz, 1H), 6.95-6.91 (m, 1H), 6.87 (s, 1H), 5.31 (s, 2H), 4.60-4.53 (m, 1H), 3.73 (d, J = 3.2 Hz, 6H), 2.82-2.71 (m, 1H), 2.67-2.53 (m, 2H), 2.23- 2.13 (m, 1H). ¹⁹F NMR (376 MHz, d₆-DMSO) δ −120.174.LCMS: calc. for C₂₄H₂₁FN₄O₅: 464.1, found: [M + H]⁺ 465.4. |
| 123 | 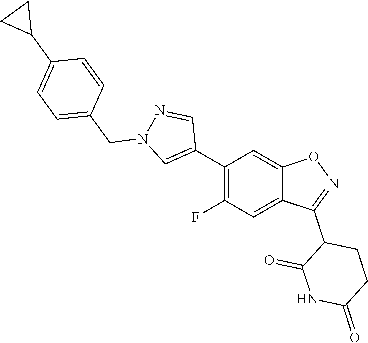 [SMILES: O=C1CCC(c2noc3cc(-c4cnn(Cc5ccc(C6CC6)cc5)c4)c(F)cc23)C(=O)N1] | ¹H NMR (400 MHz, DMSO-d₆) δ 11.09 (s, 1H), 8.41 (d, J = 2.4 Hz, 1H), 8.15 (d, J = 5.6 Hz, 1H), 8.11 (s, 1H), 7.83 (d, J = 10.4 Hz, 1H), 7.20 (d, J = 8.0 Hz, 2H), 7.05 (d, J = 8.0 Hz, 2H), 5.33 (s, 2H), 4.57 (dd, J = 4.8, 12.0 Hz, 1H), 2.82-2.71 (m, 1H), 2.65-2.54 (m, 2H), 2.19 (dd, J = 4.0, 8.8 Hz, 1H), 1.93-1.84 (m, 1H), 0.96-0.88 (m, 2H), 0.68- 0.58 (m, 2H). ¹⁹F NMR (376 MHz, DMSO-d₆) δ −120.159.LCMS: calc. for C₂₅H₂₁FN₄O₃: 444.1, found: [M + H]⁺ 445.3. |
| 124 | 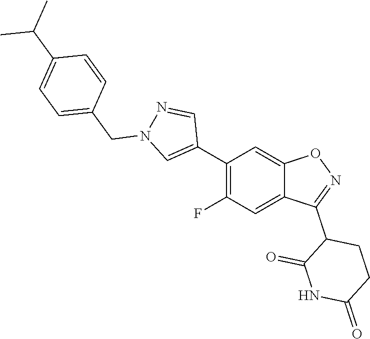 [SMILES: CC(C)c1ccc(Cn2cc(-c3cc4onc(C5CCC(=O)NC5=O)c4cc3F)cn2)cc1] | ¹H NMR (400 MHz, d₆-DMSO) 8.44 (d, J = 4.0 Hz, 1H), 8.19-8.09 (m, 2H), 7.83 (d, J = 10.4 Hz, 1H), 7.27-7.20 (m, 4H), 5.39-5.32 (m, 2H), 4.61- 4.52 (m, 1H), 2.90-2.83 (m, 1H), 2.78-2.71 (m, 1H), 2.68-2.54 (m, 2H), 2.23-2.14 (m, 1H), 1.17 (d, J = 6.8 Hz, 6H).LCMS: calc. for C₂₅H₂₃FN₄O₃: 446.2, found: [M + H]⁺ 447.4. |
| 125 | 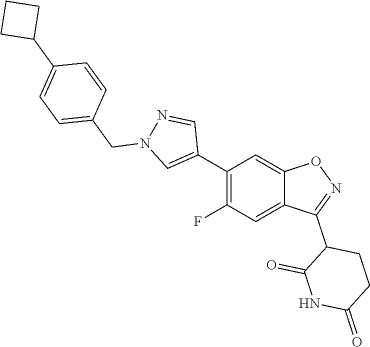 [SMILES: O=C1CCC(c2noc3cc(-c4cnn(Cc5ccc(C6CCC6)cc5)c4)c(F)cc23)C(=O)N1] | ¹H NMR (400 MHz, DMSO-d₆) δ 11.09 (s, 1H), 8.42 (d, J = 2.8 Hz, 1H), 8.15 (d, J = 5.6 Hz, 1H), 8.11 (s, 1H), 7.83 (d, J = 10.4 Hz, 1H), 7.23 (q, J = 8.0 Hz, 4H), 5.36 (s, 2H), 4.57 (dd, J = 4.8, 12.0 Hz, 1H), 3.49 (quin, J = 8.8 Hz, 1H), 2.82-2.71 (m, 1H), 2.64-2.55 (m, 2H), 2.30-2.22 (m, 2H), 2.21-2.14 (m, 1H), 2.10-2.00 (m, 2H), 1.99- 1.91 (m, 1H), 1.83-1.74 (m, 1H). ¹⁹F NMR (376 MHz, DMSO-d₆) δ −120.155.LCMS: calc. for C₂₆H₂₃FN₄O₃: 458.2, found: [M + H]⁺ 459.4. |
| 481 | 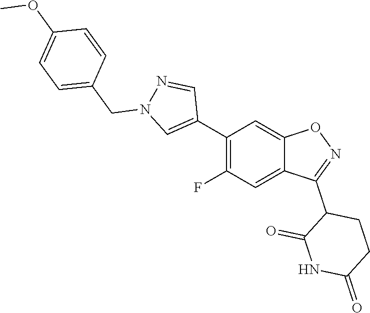 [SMILES: COc1ccc(Cn2cc(-c3cc4onc(C5CCC(=O)NC5=O)c4cc3F)cn2)cc1] | ¹H NMR (400 MHz, d₆-DMSO) δ 11.10 (br s, 1H), 8.39 (d, J = 2.8 Hz, 1H), 8.15 (d, J = 5.6 Hz, 1H), 8.11 (s, 1H), 7.83 (d, J-10.4 Hz, 1H), 7.29 (d, J = 8.4 Hz, 2H), 6.92 (d, J = 8.8 Hz, 2H), 5.32 (s, 2H), 4.57 (dd, J = 4.8, 12.0 Hz, 1H), 3.73 (s, 3H), 2.82-2.71 (m, 1H), 2.64-2.52 (m, 2H), 2.24-2.13 (m, 1H). MS: (ESI) m/z calculated for C₂₃H₁₉FN₄O₄ [M + H]⁺ 435.1, found 435.0. |
| 482 | 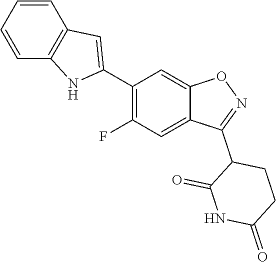 [SMILES: O=C1CCC(c2noc3cc(-c4cc5ccccc5[nH]4)c(F)cc23)C(=O)N1] | ¹H NMR (400 MHz, d₆-DMSO) δ 11.69 (s, 1H), 11.21-11.07 (m, 1H), 8.28 (d, J-8.0 Hz, 1H), 7.95 (d, J = 10.8 Hz, 1H), 7.63 (d, J = 8.0 Hz, 1H), 7.48 (d, J = 8.2 Hz, 1H), 7.19 (t, J = 8.0 Hz, 1H), 7.12 (br s, 1H), 7.08-7.03 (m, 1H), 4.62 (dd, J = 4.8, 12.0 Hz, 1H), 2.84-2.73 (m, 1H), 2.65-2.59 (m, 2H), 2.25-2.16 (m, 1H). MS: (ESI) m/z calculated for C₂₀H₁₄FN₃O₃ [M + H]⁺ 364.1, found 364.1. |
| 483 | 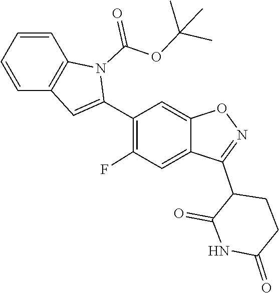 [SMILES: CC(C)(C)OC(=O)n1c(-c2cc3onc(C4CCC(=O)NC4=O)c3cc2F)cc2ccccc21] | ¹H NMR (400 MHz, d₆-DMSO) δ 11.19- 11.04 (m, 1H), 8.14 (d, J = 8.8 Hz, 1H), 8.03 (d, J = 5.6 Hz, 1H), 7.89 (d, J = 9.2 Hz, 1H), 7.69 (d, J = 7.6 Hz, 1H), 7.43-7.38 (m, 1H), 7.33-7.28 (m, 1H), 6.96 (s, 1H), 4.64 (dd, J = 5.2, 12.4 Hz, 1H), 2.85-2.73 (m, 1H), 2.68- 2.57 (m, 2H), 2.24-2.16 (m, 1H), 1.29 (s, 9H). MS: (ESI) m/z calculated for C₂₅H₂₂FN₃O₅ [M + H]⁺ 464.2, found 464.3. |
| 484 | 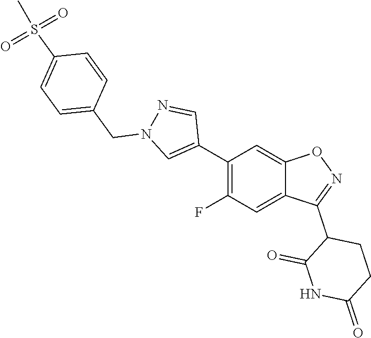 [SMILES: CS(=O)(=O)c1ccc(Cn2cc(-c3cc4onc(C5CCC(=O)NC5=O)c4cc3F)cn2)cc1] | ¹H NMR (400 MHz, d₆-DMSO) δ 11.11 (s, 1H), 8.52 (d, J = 2.8 Hz, 1H), 8.18 (d, J = 3.2 Hz, 2H), 7.92 (d, J = 8.0 Hz, 2H), 7.85 (d, J = 10.8 Hz, 1H), 7.53 (d, J = 8.0 Hz, 2H), 5.56 (s, 2H), 4.57 (dd, J = 4.8, 12.0 Hz, 1H), 3.19 (s, 3H), 2.82-2.71 (m, 1H), 2.63-2.56 (m, 2H), 2.24-2.14 (m, 1H). ¹⁹F NMR (376 MHz, d₆- DMSO) δ −120.08. MS: (ESI) m/z [M + H]⁺ 483.3. |
| 485 | 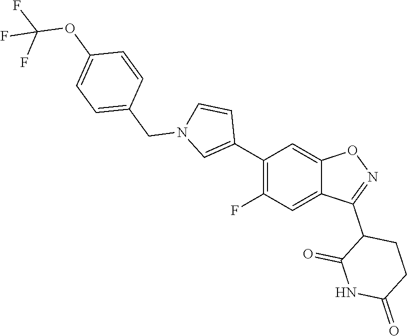 [SMILES: O=C1CCC(c2noc3cc(-c4ccn(Cc5ccc(OC(F)(F)F)cc5)c4)c(F)cc23)C(=O)N1] | ¹H NMR (400 MHz, d₆-DMSO) δ 11.11- 11.06 (m, 1H), 8.00 (d, J = 8.0 Hz, 1H), 7.73 (d, J = 11.2 Hz, 1H), 7.53 (m, 1H), 7.43-7.33 (m, 4H), 7.01 (m, 1H), 6.72 (m, 1H), 5.23 (s, 2H), 4.54 (dd, J = 4.8, 12.4 Hz, 1H), 2.64- 2.60 (m, 1H), 2.59-2.56 (m, 1H), 2.43-2.38 (m, 1H), 2.22-2.14 (m, 1H). ¹⁹F NMR (376 MHz, d₆-DMSO) δ −56.8, −119.9. MS: (ESI) m/z [M + H]⁺ 488.1. |
| 486 | 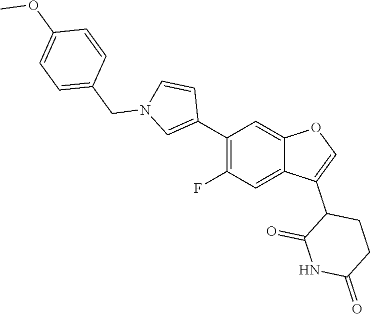 [SMILES: COc1ccc(Cn2ccc(-c3cc4occ(C5CCC(=O)NC5=O)c4cc3F)c2)cc1] | ¹H NMR (400 MHz, METHANOL-d₄) δ 10.61-10.30 (m, 1H), 7.80-7.74 (m, 1H), 7.50-7.43 (m, 1H), 7.27-7.23 (m, 1H), 7.20- 7.14 (m, 2H), 6.89-6.82 (m, 2H), 6.38- 6.34 (m, 1H), 4.48-4.42 (m, 1H), 3.90 (s, 2H), 3.76 (s, 3H), 2.80 (dd, J = 5.2, 7.8 Hz, 2H), 2.55 (tdd, J = 7.8, 10.8, 13.6 Hz, 1H), 2.40-2.31 (m, 1H). MS: (ESI) m/z [M + H]⁺ 434.3. |
| 487 | 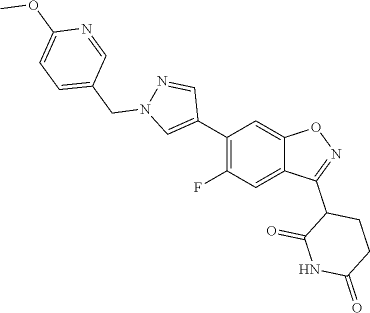 [SMILES: COc1ccc(Cn2cc(-c3cc4onc(C5CCC(=O)NC5=O)c4cc3F)cn2)cn1] | ¹H NMR (400 MHz, d₆-DMSO) δ 11.10 (s, 1H), 8.44 (d, J = 2.6 Hz, 1H), 8.22 (d, J = 2.4 Hz, 1H), 8.14 (d, J = 5.6 Hz, 1H), 8.11 (s, 1H), 7.83 (d, J = 10.5 Hz, 1H), 7.70 (dd, J = 2.4, 8.4 Hz, 1H), 6.82 (d, J = 8.5 Hz, 1H), 5.35 (s, 2H), 4.56 (dd, J = 4.8, 12.0 Hz, 1H), 3.83 (s, 3H), 2.79-2.71 (m, 1H), 2.64-2.61 (m, 1H), 2.60-2.57 (m, 1H), 2.23-2.15 (m, 1H). ¹⁹F NMR (376 MHz, d₆-DMSO) δ −120.16. MS: (ESI) m/z [M + H]⁺ 436.3. |
| 488 | 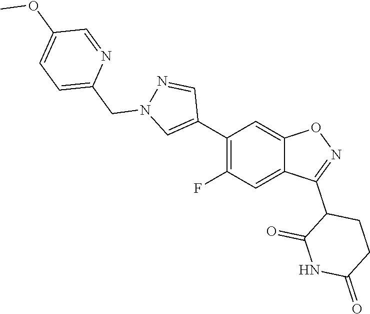 [SMILES: COc1ccc(Cn2cc(-c3cc4onc(C5CCC(=O)NC5=O)c4cc3F)cn2)nc1] | ¹H NMR (400 MHz, d₆-DMSO) δ 11.10 (s, 1H), 8.42 (d, J = 2.8 Hz, 1H), 8.25 (d, J = 2.8 Hz, 1H), 8.16 (d, J = 5.6 Hz, 1H), 8.12 (s, 1H), 7.83 (d, J = 10.4 Hz, 1H), 7.39 (dd, J = 2.8, 8.0 Hz, 1H), 7.23 (d, J = 8.0 Hz, 1H), 5.43 (s, 2H), 4.57 (dd, J = 4.0, 12.0 Hz, 1H), 3.81 (s, 3H), 2.77-2.72 (m, 1H), 2.62 (m, 1H), 2.59 (m, 1H), 2.23-2.15 (m, 1H). ¹⁹F NMR (376 MHz, d₆-DMSO) δ 120.12. MS: (ESI) m/z [M + H]⁺ 436.1. |
| 489 | 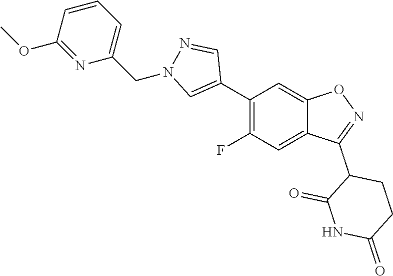 [SMILES: COc1cccc(Cn2cc(-c3cc4onc(C5CCC(=O)NC5=O)c4cc3F)cn2)n1] | ¹H NMR (400 MHz, d₆-DMSO) δ.54-8.46 (m, 1H), 8.22-8.11 (m, 2H), 7.85 (d, J = 10.6 Hz, 1H), 7.68 (t, J = 7.8 Hz, 1H), 6.81-6.62 (m, 2H), 5.43 (s, 2H), 4.57 (dd, J = 4.9, 12.0 Hz, 1H), 3.85-3.79 (m, 3H), 2.81 (br d, J = 5.0 Hz, 1H), 2.69-2.56 (m, 2H), 2.24-2.14 (m, 1H). MS: (ESI) m/z [M + H]⁺ 436.2. |
| 490 | 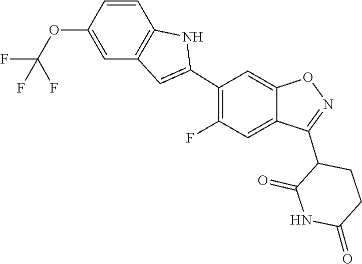 [SMILES: O=C1CCC(c2noc3cc(-c4cc5cc(OC(F)(F)F)ccc5[nH]4)c(F)cc23)C(=O)N1] | ¹H NMR (400 MHz, d₆-DMSO) δ 11.98 (s, 1H), 11.13 (br s, 1H), 8.29 (d, J = 6.0 Hz, 1H), 7.97 (d, J = 10.8 Hz, 1H), 7.64 (s, 1H), 7.56 (d, J = 8.8 Hz, 1H), 7.21-7.13 (m, 2H), 4.62 (dd, J = 4.8, 12.0 Hz, 1H), 2.82-2.73 (m, 1H), 2.60 (br s, 1H), 2.36-2.30 (m, 1H), 2.26- 2.16 (m, 1H). ¹⁹F NMR (376 MHz, d₆- DMSO) δ −56.92, −119.59. MS: (ESI) m/z [M + H]⁺ 448.3. |
| 491 | 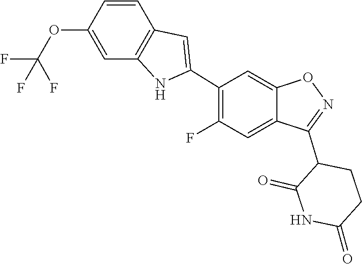 [SMILES: O=C1CCC(c2noc3cc(-c4cc5ccc(OC(F)(F)F)cc5[nH]4)c(F)cc23)C(=O)N1] | ¹H NMR (400 MHz, d₆-DMSO) δ 11.96 (s, 1H), 11.13 (s, 1H), 8.28 (d, J = 5.6 Hz, 1H), 7.97 (d, J = 10.8 Hz, 1H), 7.73 (d, J = 8.4 Hz, 1H), 7.42 (s, 1H), 7.18 (br s, 1H), 7.05 (dd, J = 1.2, 8.8 Hz, 1H), 4.62 (dd, J = 5.2, 12.0 Hz, 1H), 2.85-2.72 (m, 1H), 2.68-2.63 (m, 1H), 2.57 (br d, J = 4.4 Hz, 1H), 2.26-2.16 (m, 1H) ¹⁹F NMR (376 MHz, d₆-DMSO) δ −56.75, −119.71. MS: (ESI) m/z [M + H]⁺ 448.3. |
| 492 | 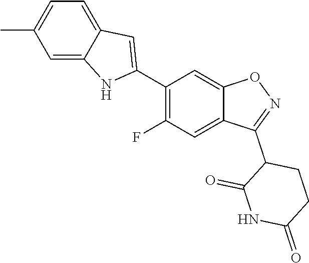 [SMILES: Cc1ccc2cc(-c3cc4onc(C5CCC(=O)NC5=O)c4cc3F)[nH]c2c1] | ¹H NMR (400 MHz, DMSO-d₆) δ 11.52 (s, 1H), 11.12 (br s, 1H), 8.25 (d, J = 6.0 Hz, 1H), 7.92 (d, J = 11.2 Hz, 1H), 7.50 (d, J = 8.4 Hz, 1H), 7.26 (s, 1H), 7.06 (br s, 1H), 6.89 (d, J = 8.0 Hz, 1H), 4.61 (dd, J = 4.8, 12.0 Hz, 1H), 2.87-2.70 (m, 2H), 2.43 (s, 3H), 2.25-2.16 (m, 1H), 2.05-1.94 (m, 1H). ¹⁹F NMR (376 MHz, DMSO-d₆) δ −119.46. MS: (ESI) m/z [M + H]⁺ 378.3 |
| 493 | 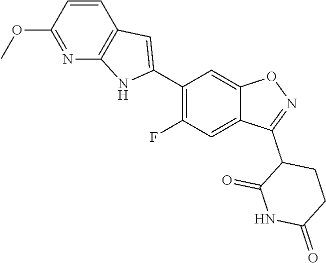 [SMILES: COc1ccc2cc(-c3cc4onc(C5CCC(=O)NC5=O)c4cc3F)[nH]c2n1] | ¹H NMR (400 MHz, DMSO-d₆) δ 12.05 (s, 1H), 11.08 (br s, 1H), 8.12 (d, J = 4.4 Hz, 1H), 8.01 (s, 1H), 7.86 (d, J = 12.0 Hz, 1H), 7.68 (s, 1H), 6.65 (s, 1H), 4.62-4.58 (m, 1H), 3.91 (s, 3H), 2.79-2.75 (m, 1H), 2.65-2.64 (m, 2H), 2.22-2.19 (m, 1H). ¹⁹F NMR (376 MHz, d₆-DMSO) δ −120.03. MS: (ESI) m/z [M + H]⁺ 395.3. |
| 494 | 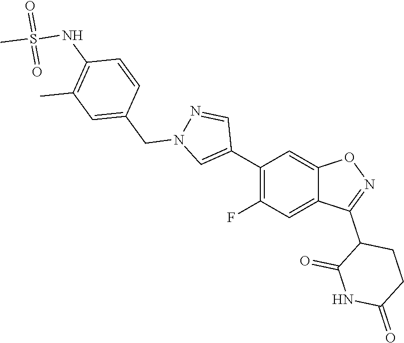 [SMILES: Cc1cc(Cn2cc(-c3cc4onc(C5CCC(=O)NC5=O)c4cc3F)cn2)ccc1NS(C)(=O)=O] | ¹H NMR (400 MHz, DMSO-d₆) δ 11.15 (s, 1H), 9.12 (br s, 1H), 8.45 (s, 1H), 8.14 (d, J = 10.8 Hz, 1H), 8.11 (s, 1H), 7.84 (d, J = 6.4 Hz, 1H), 7.26-7.21 (m, 2H), 7.14 (d, J = 8.0 Hz, 1H), 5.35 (s, 2H), 4.55-4.56 (m, 1H), 2.95 (s, 3H), 2.62-2.59 (m, 3H), 2.25 (s, 3H), 2.20- 2.19 (m, 1H). ¹⁹F NMR (376 MHz, d₆- DMSO) δ −120.10. MS: (ESI) m/z [M + H]⁺ 512.4. |
| 495 | 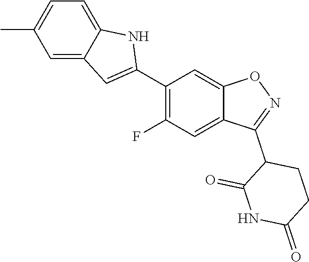 [SMILES: Cc1ccc2[nH]c(-c3cc4onc(C5CCC(=O)NC5=O)c4cc3F)cc2c1] | ¹H NMR (400 MHz, d₆-DMSO) δ 11.56 (s, 1H), 11.12 (s, 1H), 8.25 (d, J = 8.0 Hz, 1H), 7.92 (d, J = 12.0 Hz, 1H), 7.40 (s, 1H), 7.37 (d, J = 8.4 Hz, 1H), 7.03 (br s, 1H), 7.02 (br d, J = 4.0 Hz, 1H), 4.61 (dd, J = 5.2, 12.0 Hz, 1H), 2.82-2.74 (m, 1H), 2.64 (br d, J = 4.0 Hz, 1H), 2.60 (br s, 1H), 2.40-2.37 (m, 3H), 2.24-2.17 (m, 1H). ¹⁹F NMR (376 MHz, d₆- DMSO) δ −119.47. MS: (ESI) m/z [M + H]⁺ 378.3. |
| 496 | 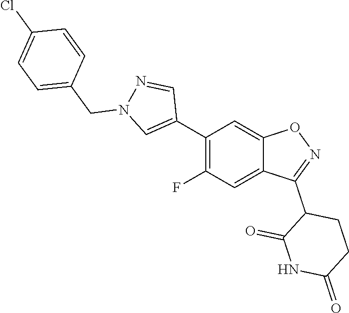 [SMILES: O=C1CCC(c2noc3cc(-c4cnn(Cc5ccc(Cl)cc5)c4)c(F)cc23)C(=O)N1] | ¹H NMR (400 MHz, d₆-DMSO) 8.49-8.44 (m, 1H), 8.18-8.13 (m, 2H), 7.88-7.80 (m, 1H), 7.48-7.40 (m, 2H), 7.33 (d, J = 8.4 Hz, 2H), 5.42 (s, 2H), 4.63-4.52 (m, 1H), 2.83- 2.71 (m, 1H), 2.68-2.56 (m, 2H), 2.23-2.14 (m, 1H). MS: (ESI) m/z [M + H]⁺ 439.2. |
| 497 | 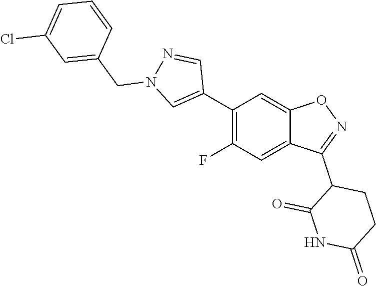 [SMILES: O=C1CCC(c2noc3cc(-c4cnn(Cc5cccc(Cl)c5)c4)c(F)cc23)C(=O)N1] | ¹H NMR (400 MHz, d₆-DMSO) 8.49 (d, J = 2.8 Hz, 1H), 8.19-8.13 (m, 2H), 7.84 (d, J = 10.4 Hz, 1H), 7.43-7.35 (m, 3H), 7.29-7.23 (m, 1H), 5.43 (s, 2H), 4.57 (dd, J = 4.9, 12.0 Hz, 1H), 2.74-2.61 (m, 2H), 2.35-2.15 (m, 2H). MS: (ESI) m/z [M + H]⁺ 439.1. |

### Example 126:3-(6-(2-(4-methoxybenzyl) oxazol-5-yl)benzofuran-3-yl)piperidine-2,6-dione

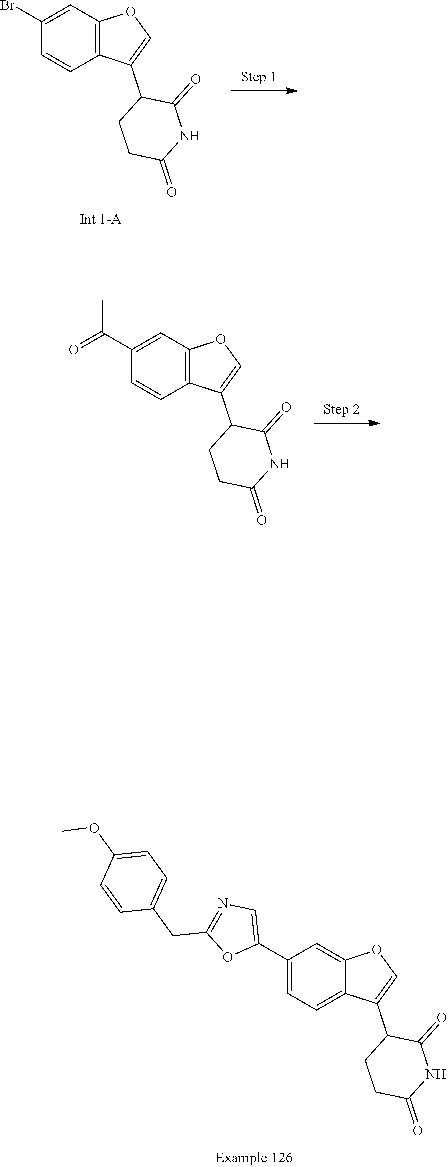 [SMILES: O=C1CCC(c2coc3cc(Br)ccc23)C(=O)N1 ; CC(=O)c1ccc2c(C3CCC(=O)NC3=O)coc2c1 ; COc1ccc(Cc2ncc(-c3ccc4c(C5CCC(=O)NC5=O)coc4c3)o2)cc1]

Step 1: To a solution of 3-(6-bromobenzofuran-3-yl)piperidine-2,6-dione (Int 1, 300 mg, 973.62 μmol, 1 eq) and Pd(PPh3)₂Cl₂ (68.34 mg, 97.36 μmol, 0.1 eq) in dioxane (3 mL) was added tributyl(1-ethoxyvinyl) stannane (386.78 mg, 1.07 mmol, 361.48 μL, 1.1 eq). The mixture was stirred at 85° C. for 16 h under N₂. The mixture was quenched with saturated aqueous KF solution and stirred at 20° C. for 0.5 h. The solution was filtered and the filtrate was extracted with EtOAc, the combined organic layers were dried over anhydrous Na₂SO₄, filtered and concentrated under reduced pressure. The residue was purified by silica gel column chromatography to afford 3-(6-acetylbenzofuran-3-yl)piperidine-2,6-dione (120 mg, 415.83 μmol, 42.71% yield, 94% purity) as a white solid. ¹H NMR (400 MHZ, CD₃OD)_{8 8.16} (d, J=0.8 Hz, 1H), 7.89-8.01 (m, 2H), 7.69 (d, J=8.4 Hz, 1H), 4.19 (dd, J=12.0, 5.2 Hz, 1H), 2.73-2.89 (m, 2H), 2.67 (s, 3H), 2.36-2.48 (m, 1H), 2.23-2.34 (m, 1H). LCMS: calc. for C₁₅H₁₃NO₄:271.1, found: [M+H]⁺272.2.

Step 2: To a solution of 3-(6-acetylbenzofuran-3-yl)piperidine-2,6-dione (50 mg, 184.32 μmol, 1 eq) in DMSO (3 mL) was added I₂ (93.56 mg, 368.64 μmol, 74.26 μL, 2 eq). The mixture was stirred at 110° C. for 45 min under N₂, and then 2-amino-3-(4-methoxyphenyl) propanoic acid (71.96 mg, 368.64 μmol, 2 eq) was added. The resulting mixture was stirred at 110° C. for 15 min. The mixture was poured into water, added in aq. Na₂SO₃ and extracted with EtOAc. The combined organic layers were washed with brine, dried over Na₂SO₄, filtered and concentrated to give a residue. The residue was purified by prep-HPLC and dried by lyophilization to afford Example 126 (4.5 mg, 10.59 μmol, 5.75% yield, 98% purity) was a white solid. ¹H NMR (400 MHZ, DMSO-d₆) δ 10.92 (s, 1H), 7.97 (s, 1H), 7.85 (s, 1H), 7.66 (d, J=8.4 Hz, 1H), 7.62 (s, 1H), 7.56 (dd, J=8.4, 1.2 Hz, 1H), 7.28 (d, J=8.8 Hz, 2H), 6.91 (d, J=8.8 Hz, 2H), 4.15-4.19 (m, 1H), 4.14 (s, 2H), 3.73 (s, 3H), 2.68-2.83 (m, 1H), 2.53-2.59 (m, 1H), 2.25-2.40 (m, 1H), 2.08-2.19 (m, 1H). LCMS: calc. for C₂₄H₂ON₂O₅:416.1, found: [M+H]⁺417.2.

### Example 127:3-[6-[1-[(5-methoxy-2-pyridyl)methyl]pyrazol-4-yl]-5-methyl-benzofuran-3-yl]piperidine-2,6-dione

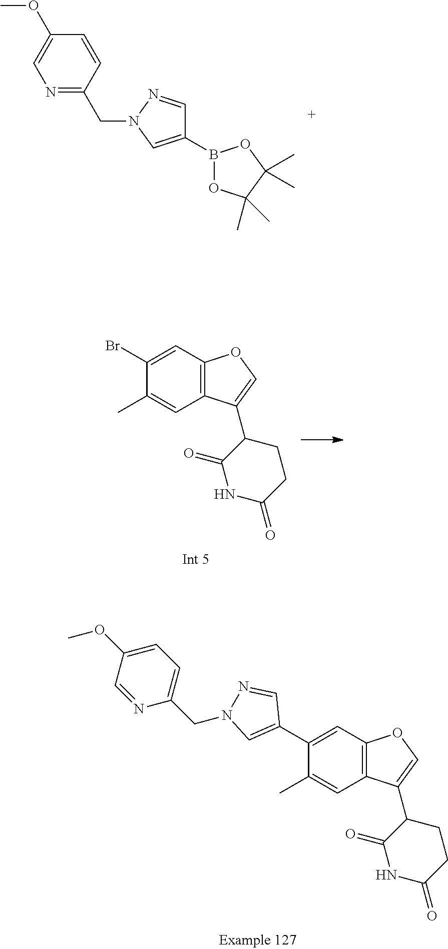

To a solution of 5-methoxy-2-[[4-(4,4,5,5-tetramethyl-1,3,2-dioxaborolan-2-yl)pyrazol-1-yl]methyl]pyridine (50 mg, 158.64 μmol, 1 eq) and Int 5 (51.11 mg, 158.64 μmol, 1 eq) in dioxane (1 mL) and H₂O (1 mL) was added K₃PO₄ (50.51 mg, 237.96 μmol, 1.5 eq) and XPhos Pd G3 (13.43 mg, 15.86 μmol, 0.1 eq). The mixture was stirred at 80° C. for 0.5 h. The reaction mixture was concentrated. The residue was purified by prep-HPLC to give Example 127 (22.4 mg, 32.38% yield) as a white solid. ¹H NMR (400 MHZ, DMSO-d₆) § 10.89 (br s, 1H), 8.25 (d, J=2.4 Hz, 1H), 8.12 (s, 1H), 7.83 (s, 1H), 7.76 (s, 1 H), 7.56 (s, 1H), 7.45 (s, 1H), 7.39 (dd, J=8.0, 2.8 Hz, 1H), 7.16 (d, J=8.0 Hz, 1H), 5.40 (s, 2H), 4.11 (dd, J=12.0, 8.0 Hz, 1H), 3.81 (s, 3H), 2.73 (dd, J=12.0, 8.0 Hz, 1H), 2.59 (d, J=4.0 Hz, 1H), 2.43 (s, 3H), 2.33 (br s, 1H), 2.36-2.31 (m, 1H), 2.16-2.06 (m, 1H). LCMS: calc. for C₂₄H₂₂N₄O₄:430.1, found: [M+H]⁺431.1.

### Example 128:3-[7-fluoro-6-[1-[(4-methoxyphenyl)methyl]pyrazol-4-yl]benzofuran-3-yl]piperidine-2,6-dione

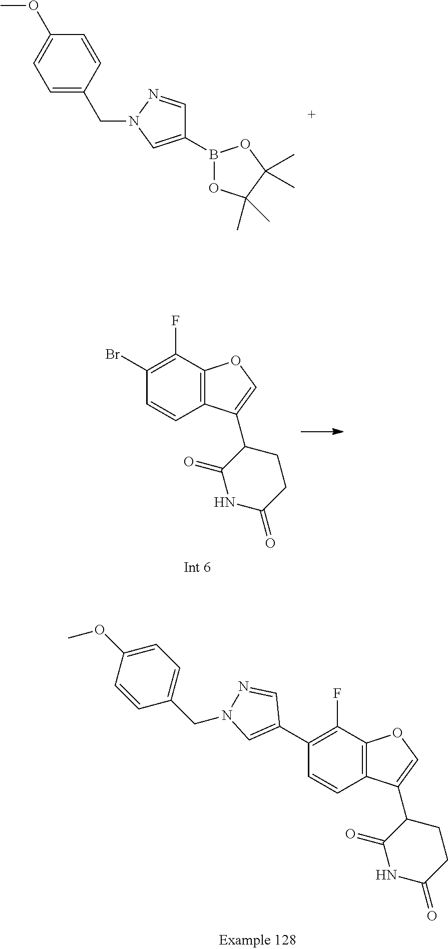

To a mixture of Int 6 (50 mg, 153.32 μmol, 1 eq) in dioxane (2 mL) and H₂O (0.5 mL) was added 1-[(4-methoxyphenyl)methyl]-4-(4,4,5,5-tetramethyl-1,3,2-dioxaborolan-2-yl)pyrazole (43.35 mg, 137.99 μmol, 0.9 eq), Xphos Pd G3 (12.98 mg, 15.33 μmol, 0.1 eq) and K₃PO₄ (48.82 mg, 229.98 μmol, 1.5 eq). The mixture was degassed and purged with N₂ for 3 times, and then was stirred at 80° C. for 1 h under N₂ atmosphere. The mixture was diluted with H₂O (30 mL), then was extracted with EtOAc (20 mL×2). The organic layer was washed with brine (30 mL), dried over Na₂SO₄, filtered, and concentrated under reduced pressure to give a residue. The residue was purified by prep-HPLC to give Example 128 (1.6 mg, 2.38% yield) as a white solid. ¹H NMR (400 MHZ, DMSO-d₆) δ 10.92 (s, 1H), 8.29 (d, J=1.2 Hz, 1H), 7.97 (d, J=12.0 Hz, 2H), 7.55 (dd, J=6.4, 8.0 Hz, 1H), 7.40 (d, J=8.4 Hz, 1H), 7.28 (d, J=8.0 Hz, 2H), 6.91 (d, J=8.0 Hz, 2H), 5.31 (s, 2H), 4.16 (dd, J=4.8, 12.0 Hz, 1H), 3.73 (s, 3H), 2.80-2.69 (m, 1H), 2.62-2.55 (m, 1H), 2.35-2.32 (m, 1H), 2.15-2.14 (m, 1H). LCMS: calc. for C₂₄H₂OFN₃O₄:433.1, found: [M+H]⁺434.1.

### Example 129:3-[7-fluoro-6-[1-[(5-methoxy-2-pyridyl)methyl]pyrazol-4-yl]benzofuran-3-yl]piperidine-2,6-dione

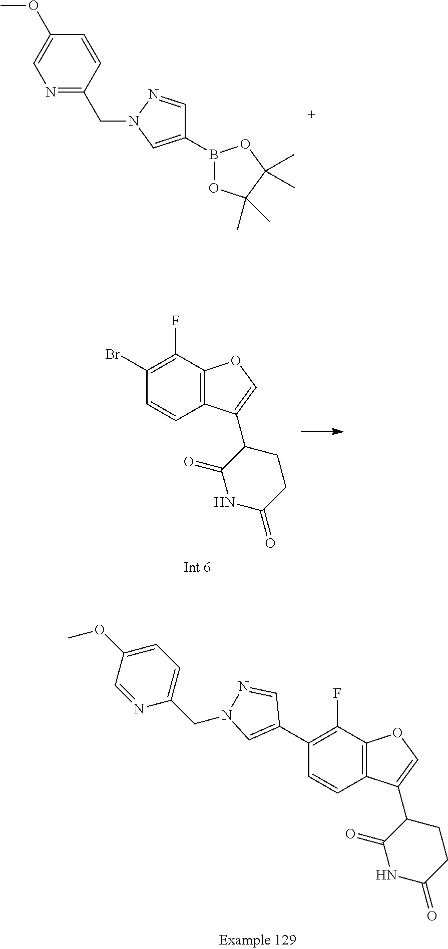

To a mixture of Int 6 (60 mg, 183.98 μmol, 1 eq) in dioxane (2.5 mL) and H₂O (0.5 mL) was added 5-methoxy-2-[[4-(4,4,5,5-tetramethyl-1,3,2-dioxaborolan-2-yl)pyrazol-1-yl]methyl]pyridine (57.99 mg, 183.98 μmol, 1 eq), K₃PO₄ (58.58 mg, 275.97 μmol, 1.5 eq) and Xphos Pd G3 (15.57 mg, 18.40 μmol, 0.1 eq). The mixture was degassed and purged with N₂ for 3 times, and then was stirred at 80° C. for 1 h under N₂ atmosphere. The mixture was diluted with H₂O (30 mL), then was extracted with EtOAc (20 mL×2). The organic layer was washed with brine (30 mL), dried over Na₂SO₄, filtered, and concentrated under reduced pressure to give a residue. The residue was purified by prep-HPLC to give Example 129 (7.2 mg, 8.63% yield) as a light yellow solid. ¹H NMR (400 MHZ, DMSO-d₆) δ 10.92 (s, 1H), 8.38 (s, 2H), 8.03-7.97 (m, 2H), 7.65-7.54 (m, 2H), 7.44-7.36 (m, 2H), 5.54 (s, 2H), 4.17 (dd, J=4.8, 12.0 Hz, 1H), 3.86 (s, 3H), 2.81-2.70 (m, 1H), 2.60-2.57 (m, 1H), 2.35-2.32 (m, 1H), 2.15-2.14 (m, 1H). LCMS: calc. for C₂₃H₁₉FO₄N₄:434.1, found: [M+H]⁺435.1.

### Example 246:3-(6-(1-((5-(2-(dimethylamino) ethoxy)pyrimidin-2-yl)methyl)-1H-pyrazol-4-yl)-7-fluorobenzofuran-3-yl)piperidine-2,6-dione

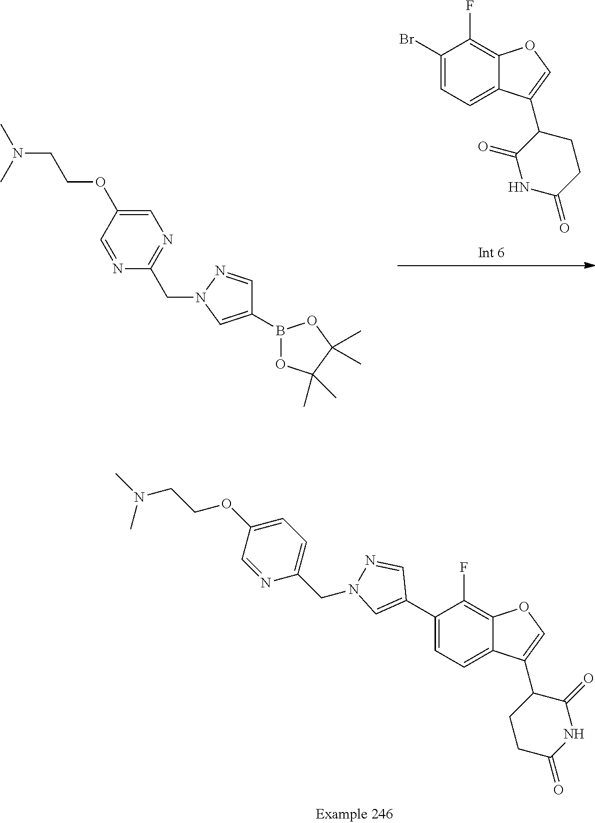

A mixture of N,N-dimethyl-2-((2-((4-(4,4,5,5-tetramethyl-1,3,2-dioxaborolan-2-yl)-1H-pyrazol-1-yl)methyl)pyrimidin-5-yl)oxy) ethan-1-amine (40 mg, 107.16 μmol, 1 eq), Int 6 (41.94 mg, 128.60 μmol, 1.2 eq), XPhos Pd G3 (9.07 mg, 10.72 μmol, 0.1 eq), K₃PO₄ (34.12 mg, 160.75 μmol, 1.5 eq) in dioxane (2 mL) and H₂O (0.5 mL) was degassed and purged with N₂ for 3 times at 20° C., and then the mixture was stirred at 80° C. for 0.5 h under N₂ atmosphere. The reaction mixture was concentrated in vacuum. The residue was purified by prep-HPLC to give Example 246 (10.8 mg, 20.04 μmol, 18.70% yield) as a white solid. ¹H NMR (400 MHZ, DMSO-d₆) § 10.94 (s, 1H), 8.55 (s, 2H), 8.35 (s, 1H), 8.00 (s, 1H), 7.96 (s, 1H), 7.61-7.59 (m, 1H), 7.43 (d, J=8.0 Hz, 1H), 5.56 (s, 2H), 4.23-4.17 (m, 3H), 2.76-2.63 (m, 1H), 2.62-2.58 (m, 3H), 2.37-2.34 (m, 1H), 2.33 (s, 6H), 2.33-2.21 (m, 1H). LCMS: calc. for C₂₅H₂₅FN₆O₄:492.2, found: [M+H]⁺493.2.

### Example 247:3-(7-fluoro-6-(1-((5-((2-methoxyethyl)(methyl)amino)pyrimidin-2-yl)methyl)-1H-pyrazol-4-yl)benzofuran-3-yl)piperidine-2,6-dione

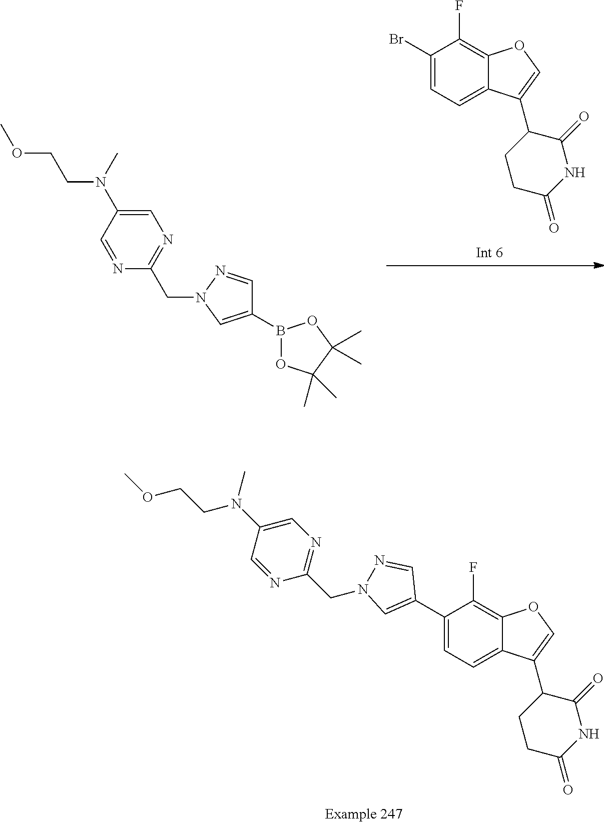

To a mixture of N-(2-methoxyethyl)-N-methyl-2-[[4-(4,4,5,5-tetramethyl-1,3,2-dioxaborolan-2-yl)pyrazol-1-yl]methyl]pyrimidin-5-amine (47 mg, 125.92 μmol, 1 eq) and Int 6 (45.17 mg, 138.51 μmol, 1.1 eq) in dioxane (2 mL) and H₂O (0.5 mL) was added K₃PO₄ (32.07 mg, 151.10 μmol, 1.2 eq) and XPhos Pd G; (10.66 mg, 12.59 μmol, 0.1 eq). The mixture was degassed and purged with N₂ for 3 times, and then was stirred at 90° C. for 2 h under N₂ atmosphere. The mixture was diluted with H₂O (10 mL), then was extracted with EtOAc (10 mL×3). The organic layer was washed with brine (10 mL), dried over Na₂SO₄, filtered and concentrated under reduced pressure to give a residue. The residue was purified by prep-HPLC and lyophilized to give Example 247 (12.1 mg, 18.85% yield) as a white solid. ¹H NMR (400 MHz, DMSO-d₆) § 10.93 (s, 1H), 8.29 (s, 1H), 8.26 (s, 2H), 8.00-7.98 (m, 1H), 7.94-7.92 (m, 1H), 7.63-7.52 (m, 1H), 7.44-7.38 (m, 1H), 5.47-5.39 (m, 2H), 4.17 (dd, J=4.8, 12.4 Hz, 1H), 3.59-3.53 (m, 2H), 3.51-3.46 (m, 2H), 3.22 (s, 3H), 2.97-2.92 (m, 3H), 2.81-2.70 (m, 1H), 2.62-2.56 (m, 1H), 2.40-2.26 (m, 1H), 2.20-2.08 (m, 1H). LCMS: calc. for C₂₅H₂₂FN₆O₄:492.1, found: [M+H]⁺493.1.

### Example 248:3-(7-fluoro-6-(1-((2-morpholinothiazol-5-yl)methyl)-1H-pyrazol-4-yl)benzofuran-3-yl)piperidine-2,6-dione

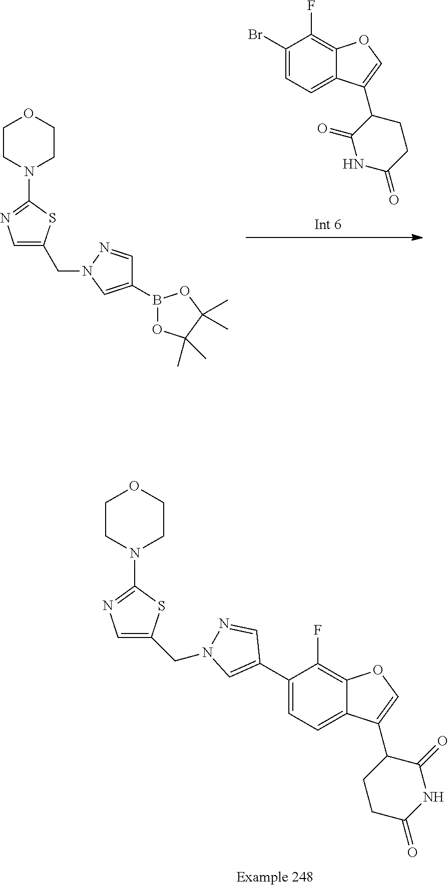 [SMILES: CC1(C)OB(c2cnn(Cc3cnc(N4CCOCC4)s3)c2)OC1(C)C ; O=C1CCC(c2coc3c(F)c(Br)ccc23)C(=O)N1 ; O=C1CCC(c2coc3c(F)c(-c4cnn(Cc5cnc(N6CCOCC6)s5)c4)ccc23)C(=O)N1]

A mixture of 4-[5-[[4-(4,4,5,5-tetramethyl-1,3,2-dioxaborolan-2-yl)pyrazol-1-yl]methyl]thiazol-2-yl]morpholine (60 mg, 159.46 μmol, 1 eq), Int 6 (62.4 mg, 191.35 μmol, 1.2 eq), XPhos Pd G3 (13.5 mg, 15.95 μmol, 0.1 eq), K₃PO₄ (50.8 mg, 239.18 μmol, 1.5 eq) in dioxane (2 mL) and H₂O (0.5 mL) was degassed and purged with N₂ for 3 times, and then the mixture was stirred at 80° C. for 0.5 h under N₂ atmosphere. The reaction mixture was concentrated. The residue was purified by prep-HPLC to give Example 248 (13.2 mg, 16.34% yield) as a white solid. ¹H NMR (400 MHZ, DMSO-d₆) δ 10.93 (s, 1H), 8.29-8.23 (m, 1H), 7.99 (s, 1H), 7.97 (s, 1H), 7.58-7.53 (m, 1H), 7.41 (d, J=8.4 Hz, 1H), 7.26 (s, 1H), 5.46 (s, 2H), 4.20-4.14 (m, 1H), 3.68-3.65 (m, 4H), 3.34 (s, 2H), 3.32-3.30 (m, 2H), 2.80-2.70 (m, 1H), 2.63-2.58 (m, 1H), 2.37-2.30 (m, 1H), 2.16-2.09 (m, 1H). LCMS: calc. for C₂₄H₂₂FN₅₀₄S: 495.1, found: [M+H]⁺496.1.

Examples 249-258 were prepared following the similar procedure that was used for preparing Examples 246-248, starting from Int-6 and their corresponding boronic acid or ester intermediates, with proper modifications. Characterizations of Examples 249-258 are provided in Table 5.

TABLE 5

| Example | | |
|---|---|---|
| ID | Structure | NMR/MS |
| 249 | 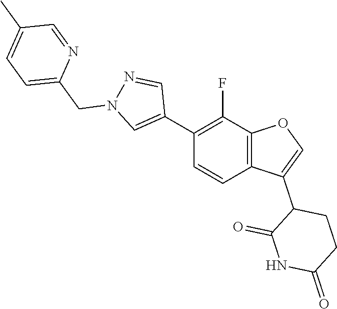 [SMILES: Cc1ccc(Cn2cc(-c3ccc4c(C5CCC(=O)NC5=O)coc4c3F)cn2)nc1] | ¹H NMR (400 MHz, DMSO-d₆) δ 10.92 (s, 1H), 8.36 (br d, J = 12.8 Hz, 2H), 7.99 (s, 2H), 7.59 (br dd, J = 7.6, 12.8 Hz, 2H), 7.41 (d, J = 8.4 Hz, 1H), 7.08 (d, J = 8.0 Hz, 1H), 5.45 (s, 2H), 4.17 (dd, J = 4.8, 12.0 Hz, 1H), 2.75-2.70 (m, 1H), 2.63-2.59 (m, 1H), 2.35 (br dd, J = 1.8, 3.2 Hz, 1H), 2.28 (s, 3H), 2.17-2.11 (m, 1H). MS: (ESI) m/z calculated for C₂₃H₁₉FN₄O₃ [M + H]⁺ 419.1, found 419.1. |
| 250 | 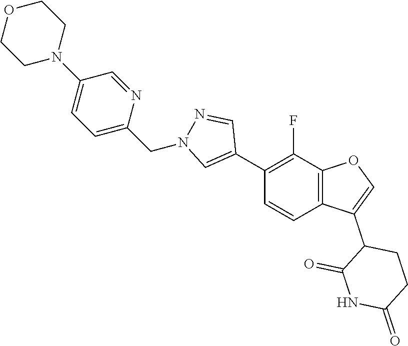 [SMILES: O=C1CCC(c2coc3c(F)c(-c4cnn(Cc5ccc(N6CCOCC6)cn5)c4)ccc23)C(=O)N1] | ¹H NMR (400 MHz, DMSO-d₆) δ 10.92 (br s, 1H), 8.38-8.22 (m, 1H), 8.20-8.03 (m, 1H), 8.02- 7.92 (m, 1H), 7.58 (q, J = 7.2 Hz, 1H), 7.41 (br d, J = 8.4 Hz, 1H), 7.37-7.20 (m, 1H), 7.12 (br d, J = 8.8 Hz, 1H), 5.38 (s, 1H), 4.17 (br dd, J = 4.4, 12.0 Hz, 1H), 3.74 (br d, J = 4.0 Hz, 2H), 3.33 (br s, 9H), 3.14 (br s, 2H), 2.84-2.69 (m, 1H), 2.67- 2.58 (m, 1H), 2.43-2.32 (m, 1H), 2.15 (br d, J = 2.9 Hz, 1H). |
| 251 | 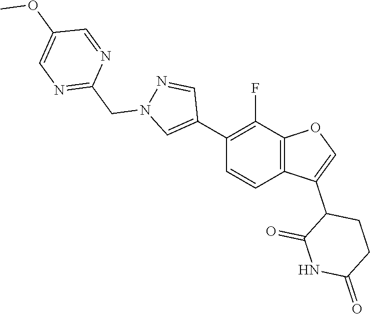 [SMILES: COc1cnc(Cn2cc(-c3ccc4c(C5CCC(=O)NC5=O)coc4c3F)cn2)nc1] | ¹H NMR (400 MHz, DMSO-d₆) δ 10.93 (s, 1H), 8.54 (s, 2H), 8.34 (d, J = 1.6 Hz, 1H), 7.97 (d, J = 14.8 Hz, 2H), 7.63-7.55 (m, 1H), 7.42 (d, J = 8.4 Hz, 1H), 5.55 (s, 2H), 4.23-4.09 (m, 1H), 3.90 (s, 3H), 2.75 (s, 1H), 2.59 (td, J = 4.0, 17.4 Hz, 1H), 2.34 ( dd, J = 4.0, 12.8 Hz, 1H), 2.18-2.08 (m, 1H). ¹⁹F NMR (376 MHz, DMSO-d₆) δ −141.047. MS: (ESI) m/z calculated for C₂₂H₁₈FN₅O₄ [M + H]⁺ 436.1, found 436.3. |
| 252 | 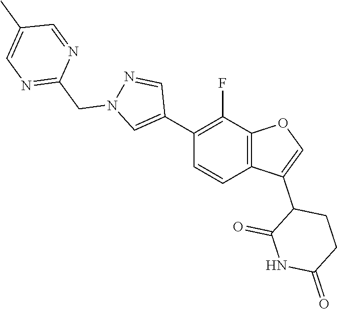 [SMILES: Cc1cnc(Cn2cc(-c3ccc4c(C5CCC(=O)NC5=O)coc4c3F)cn2)nc1] | ¹H NMR (400 MHz, DMSO-d₆) δ 10.93 (s, 1H), 8.64 (s, 2H), 8.35 (d, J = 1.6 Hz, 1H), 8.00-7.95 (m, 2H), 7.60-7.56 (m, 1H), 7.42 (d, J = 8.4 Hz, 1H), 5.57 (s, 2H), 4.19-4.15 (m, 1H), 2.80-2.71 (m, 1H), 2.62-2.56 (m, 1H), 2.40-2.31 (m, 1H), 2.26 (s, 3H), 2.18-2.10 (m, 1H). ¹⁹F NMR (376 MHz, DMSO-d₆) δ −141.03. MS: (ESI) m/z calculated for C₂₂H₁₈FN₅O₃ [M + H]⁺ 420.1, found 420.0. |
| 253 | 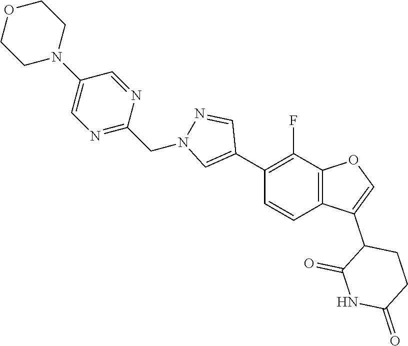 [SMILES: O=C1CCC(c2coc3c(F)c(-c4cnn(Cc5ncc(N6CCOCC6)cn5)c4)ccc23)C(=O)N1] | ¹H NMR (400 MHz, DMSO-d₆) δ 10.93 (s, 1H), 8.48 (s, 2H), 8.31 (d, J = 2.0 Hz, 1H), 7.96 (d, J = 16.0 Hz, 2H), 7.58 (dd, J = 6.4, 8.0 Hz, 1H), 7.41 (d, J = 8.4 Hz, 1H), 5.49 (s, 2H), 4.17 (dd, J = 4.8, 12.0 Hz, 1H), 3.79-3.67 (m, 4H), 3.22 (br d, J = 4.0 Hz, 4H), 2.80-2.67 (m, 1H), 2.64-2.55 (m, 1H), 2.38-2.32 (m, 1H), 2.18-2.08 (m, 1H). ¹⁹F NMR (376 MHz, DMSO-d₆) δ −141.070. MS: (ESI) m/z calculated for C₂₅H₂₃FN₆O₄ [M + H]⁺ 491.1, found 491.2. |
| 254 | 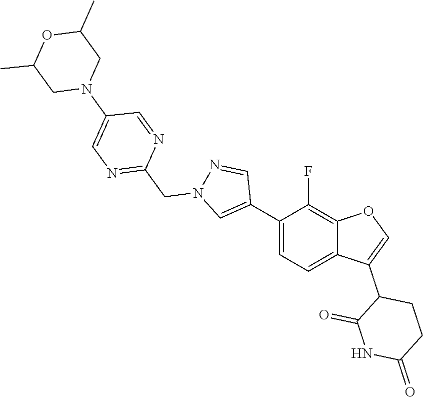 [SMILES: CC1CN(c2cnc(Cn3cc(-c4ccc5c(C6CCC(=O)NC6=O)coc5c4F)cn3)nc2)CC(C)O1] | ¹H NMR (400 MHz, DMSO-d₆) δ 10.98-10.90 (m, 1H), 8.48 (s, 2H), 8.31 (d, J = 1.2 Hz, 1H), 8.00 (s, 1H), 7.94 (s, 1H), 7.60-7.56 (m, 1H), 7.42 (d, J = 8.4 Hz, 1H), 5.48 (s, 2H), 4.19-4.15 (m, 1H), 3.76-3.63 (m, 4H), 2.80-2.71 (m, 1H), 2.64- 2.59 (m, 1H), 2.38-2.31 (m, 3H), 2.19-2.09 (m, 1H), 1.14 (d, J = 6.4 Hz, 6H). MS: (ESI) m/z calculated for C₂₇H₂₇FN₆O₄ [M + H] 519.2, found 519.1. |
| 255 | 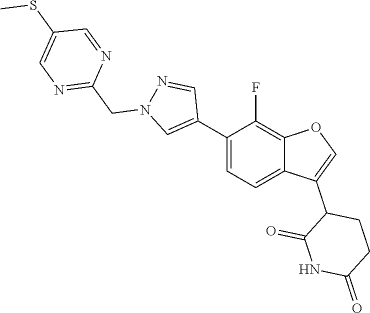 [SMILES: CSc1cnc(Cn2cc(-c3ccc4c(C5CCC(=O)NC5=O)coc4c3F)cn2)nc1] | ¹H NMR (400 MHz, DMSO-d6) δ 10.94 (s, 1H), 8.73 (s, 2H), 8.36 (s, 1H), 8.00 (d, J = 12.0 Hz, 2H), 7.61-7.56 (m, 1H), 7.43-7.41 (m, 1H), 5.59 (s, 2H), 4.20-4.15 (m, 1H), 2.75-2.72 (m, 1H), 2.62- 2.60 (m, 1H), 2.56 (s, 3H), 2.35-2.33 (m, 1H), 2.32-2.14 (m, 1H). MS: (ESI) m/z calculated for C₂₂H₁₈FN₅O₃S [M + H] 452.1, found 452.0. |
| 256 | 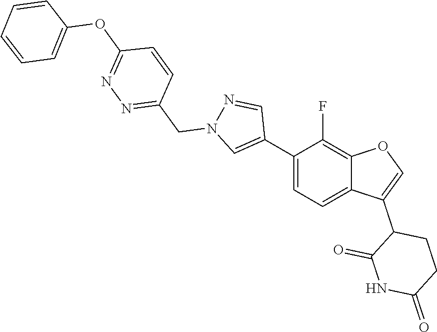 [SMILES: O=C1CCC(c2coc3c(F)c(-c4cnn(Cc5ccc(Oc6ccccc6)nn5)c4)ccc23)C(=O)N1] | ¹H NMR (400 MHz, DMSO-d₆) δ 10.96-10.90 (m, 1H), 8.43 (d, J = 1.2 Hz, 1H), 8.01 (d, J = 12.0 Hz, 2H), 7.65-7.55 (m, 2H), 7.49-7.40 (m, 4H), 7.31-7.18 (m, 3H), 5.67 (s, 2H), 4.17 (dd, J = 4.8, 12.0 Hz, 1H), 2.80-2.70 (m, 1H), 2.59 (d, J = 3.6, 17.2 Hz, 1H), 2.40-2.29 (m, 1H), 2.17-2.09 (m, 1H). MS: (ESI) m/z calculated for C₂₇H₂₀FN₅O₄ [M + H]⁺ 498.1, found 498.1. |
| 257 | 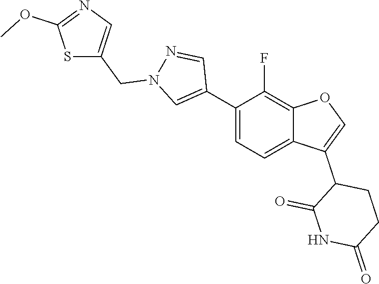 [SMILES: COc1ncc(Cn2cc(-c3ccc4c(C5CCC(=O)NC5=O)coc4c3F)cn2)s1] | ¹HNMR (400 MHz, DMSO-d₆) δ 10.93 (s, 1H), 8.30 (d, J = 1.6 Hz, 1H), 7.99 (d, J = 2.0 Hz, 2H), 7.56 (m, 1H), 7.41 (d, J = 8.4 Hz, 1H), 7.26 (s, 1H), 5.50 (s, 2H), 4.17 (m, 1H), 3.98 (s, 3H), 2.79-2.70 (m, 1H), 2.59 (m, 1H), 2.39-2.28 (m, 1H), 2.17- 2.08 (m, 1H). MS: (ESI) m/z calculated for C₂₁H₁₇FN₄O₄S [M + H] 441.1, found 441.0. |
| 258 | 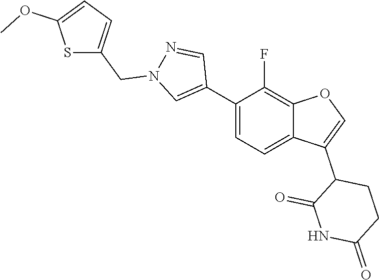 [SMILES: COc1ccc(Cn2cc(-c3ccc4c(C5CCC(=O)NC5=O)coc4c3F)cn2)s1] | ¹H NMR (400 MHz, DMSO-d₆) δ 10.93 (s, 1H), 8.32-8.22 (m, 1H), 7.98 (d, J = 5.6 Hz, 2H), 7.56 (dd, J = 6.4, 8.0 Hz, 1H), 7.41 (d, J = 8.4 Hz, 1H), 6.82 (d, J = 3.6 Hz, 1H), 6.17 (d, J = 3.6 Hz, 1H), 5.45-5.37 (m, 2H), 4.17 (dd, J = 4.8, 12.0 Hz, 1H), 3.81 (s, 3H), 2.80-2.70 (m, 1H), 2.59 (td, J = 3.6, 17.2 Hz, 1H), 2.33 (dq, J = 4.8, 12.4 Hz, 1H), 2.18-2.08 (m, 1H). MS: (ESI) m/z calculated for C₂₂H₁₈FN₃O₄S [M + H]⁺ 440.1, found 461.9. |

### Example 130:3-[4-fluoro-6-[1-[(4-methoxyphenyl)methyl]pyrazol-4-yl]benzofuran-3-yl]piperidine-2,6-dione

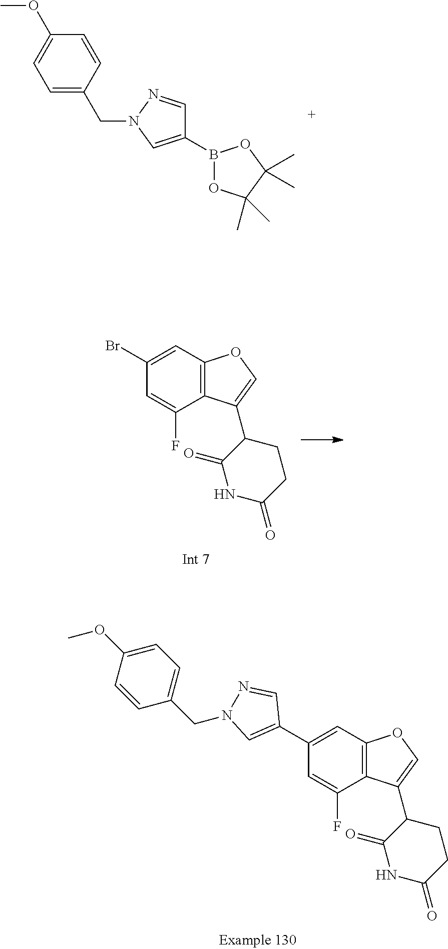

To a solution of Int 7 (100 mg, 306.64 μmol, 1 eq), 1-[(4-methoxyphenyl)methyl]-4-(4,4,5,5-tetramethyl-1,3,2-dioxaborolan-2-yl)pyrazole (105.98 mg, 337.30 μmol, 1.1 eq), K₃PO₄ (97.63 mg, 459.96 μmol, 1.5 eq) and XPhos Pd G3 (25.96 mg, 30.66 μmol, 0.1 eq) in dioxane (2 mL) and H₂O (2 mL) was degassed and purged with N₂ for 3 times, and then the mixture was stirred at 80° C. for 0.5 h under N₂ atmosphere. The reaction mixture was concentrated directly. The residue was purified by prep-HPLC to give Example 130 (39.1 mg, 29.30% yield) as a white solid. ¹H NMR (400 MHZ, DMSO-d₆) δ 10.91 (s, 1 H), 8.35 (s, 1H), 8.01 (s, 1H), 7.90 (s, 1H), 7.70 (s, 1H), 7.33 (d, J=12.0 Hz, 1H), 7.26 (d, J=8.4 Hz, 2H), 6.92 (d, J=8.0 Hz, 2H), 5.25 (s, 2H), 4.13 (dd, J=12.0, 4.8 Hz, 1H), 3.74-3.72 (m, 3H), 2.80-2.73 (m, 1H), 2.61 (td, J=2.8, 4.0 Hz, 1H), 2.23-2.17 (m, 1H), 2.13-2.05 (m, 1H). LCMS: calc. for C₂₄H₂OFN₃O₄:433.1, found: [M+H]⁺434.0.

### Example 131:3-[4-fluoro-6-[1-[(5-methoxy-2-pyridyl)methyl]pyrazol-4-yl]benzofuran-3-yl]piperidine-2,6-dione

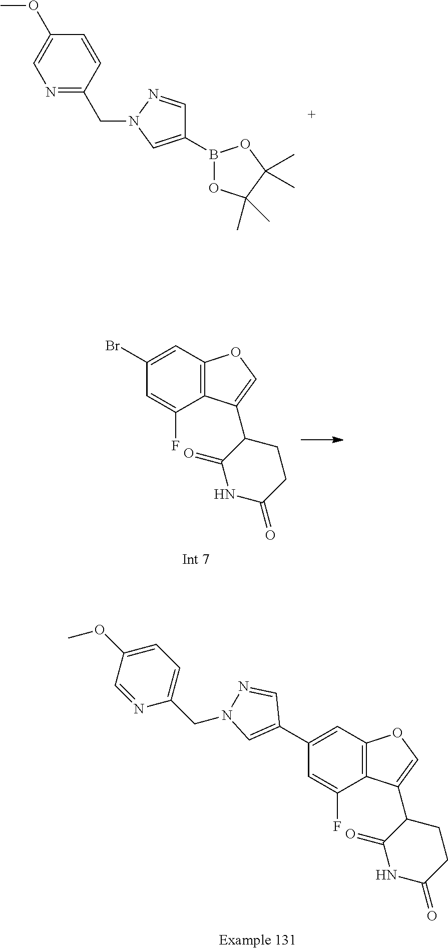

A mixture of 5-methoxy-2-[[4-(4,4,5,5-tetramethyl-1,3,2-dioxaborolan-2-yl)pyrazol-1-yl]methyl]pyridine (50 mg, 158.64 μmol, 1 eq), Int 7 (51.74 mg, 158.64 μmol, 1 eq), K₃PO₄ (50.51 mg, 237.96 μmol, 1.5 eq), and XPhos Pd G3 (13.43 mg, 15.86 μmol, 0.1 eq) in dioxane (0.8 mL) and H₂O (0.1 mL) was degassed and purged with N₂ for 3 times, and then the mixture was stirred at 80° C. for 1 h under N₂ atmosphere. Water (5 mL) was added to the reaction mixture. The reaction mixture was then adjusted to pH˜3 by aq. HCl (1 M). The resulting mixture was extracted with EtOAc (5 mL×3). The combined organic phase was washed with brine (5 mL), dried over anhydrous Na₂SO₄, filtered and concentrated. The residue was purified by prep-HPLC to give Example 131 (9.1 mg, 12.06% yield) as a white solid. ¹H NMR (400 MHZ, DMSO-d₆) § 10.92 (br s, 1H), 8.39 (s, 1H), 8.25 (d, J=2.8 Hz, 1H), 8.04 (s, 1H), 7.90 (s, 1H), 7.72 (d, J=1.2 Hz, 1H), 7.41-7.34 (m, 2H), 7.20 (d, J=8.0 Hz, 1H), 5.36 (s, 2H), 4.13 (dd, J=8.0, 12.0 Hz, 1H), 3.81 (s, 3H), 2.84-2.74 (m, 1H), 2.62-2.56 (m, 1H), 2.20 (m, 1H), 2.12-2.06 (m, 1H). LCMS: calc. for C₂₃H₁₉FO₄N₄:434.1, found: [M+H]⁺435.2.

### Example 259:3-(4-fluoro-6-(1-((5-methylpyridin-2-yl)methyl)-1H-pyrazol-4-yl)benzofuran-3-yl)piperidine-2,6-dione

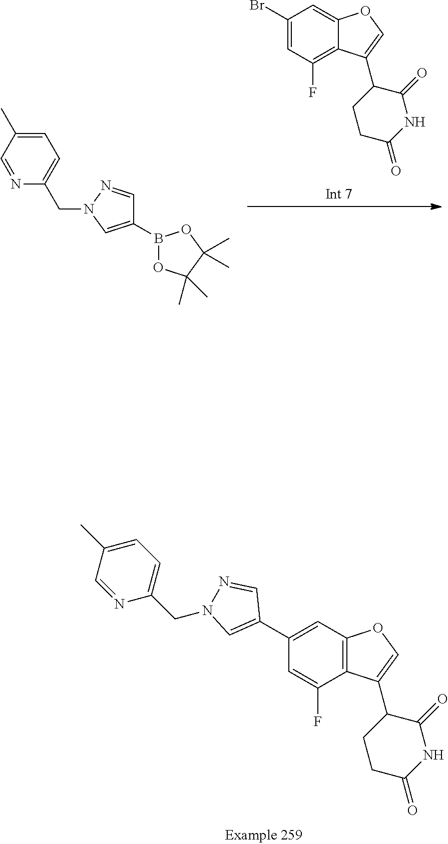 [SMILES: Cc1ccc(Cn2cc(B3OC(C)(C)C(C)(C)O3)cn2)nc1 ; O=C1CCC(c2coc3cc(Br)cc(F)c23)C(=O)N1 ; Cc1ccc(Cn2cc(-c3cc(F)c4c(C5CCC(=O)NC5=O)coc4c3)cn2)nc1]

A mixture of Int 7 (30.00 mg, 91.99 μmol, 1 eq), 5-methyl-2-[[4-(4,4,5,5-tetramethyl-1,3,2-dioxaborolan-2-yl)pyrazol-1-yl]methyl]pyridine (27.52 mg, 91.99 μmol, 1 eq), K₃PO₄ (29.29 mg, 137.99 μmol, 1.5 eq), XPhos Pd G3 (7.79 mg, 9.20 μmol, 0.1 eq) in dioxane (2 mL) and H₂O (0.5 mL) was degassed and purged with N₂ for 3 times, and then the mixture was stirred at 80° C. for 1 h under N₂ atmosphere. The reaction mixture was concentrated directly. The residue was purified by prep-HPLC and dried by lyophilization to give Example 259 (10.6 mg, 25.33 μmol, 27.54% yield) as a white solid. ¹H NMR (400 MHz, DMSO-d₆) δ 10.99-10.84 (m, 1H), 8.44-8.34 (m, 2H), 8.10-8.01 (m, 1H), 7.95-7.87 (m, 1H), 7.76-7.68 (m, 1H), 7.64-7.56 (m, 1H), 7.36 (d, J=12.0 Hz, 1H), 7.11-7.04 (m, 1H), 5.41-5.35 (m, 2H), 4.18-4.08 (m, 1H), 2.83-2.74 (m, 1H), 2.61 (s, 1H), 2.27 (s, 3H), 2.21 (dd, J=3.6, 12.0 Hz, 1H), 2.13-2.06 (m, 1H). LCMS: calc. for C₂₃H₁₉FN₄O₃:418.2, found: [M+H]⁺419.2.

Examples 260-265 were prepared following the similar procedure that was used for preparing Examples 130, 131, or 259, starting from Int-7 and their corresponding boronic acid or ester intermediates, with proper modifications. Characterizations of Examples 260-265 are provided in Table 6.

TABLE 6

| Example | | |
|---|---|---|
| ID | Structure | NMR/MS |
| 260 | 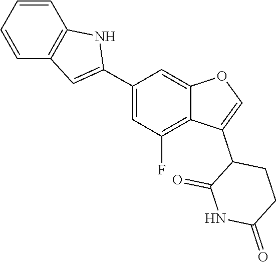 [SMILES: O=C1CCC(c2coc3cc(-c4cc5ccccc5[nH]4)cc(F)c23)C(=O)N1] | ¹H NMR (400 MHz, DMSO-d₆) δ 11.62 (s, 1H), 10.95 (s, 1H), 8.04-7.92 (m, 2H), 7.63 (dd, J = 0.8, 11.6 Hz, 1H), 7.54 (d, J = 7.6 Hz, 1H), 7.41 (d, J = 8.4 Hz, 1H), 7.12 (dt, J = 1.1, 7.6 Hz, 1H), 7.07- 6.97 (m, 2H), 4.18 (dd, J = 5.2, 12.8 Hz, 1H), 2.81 (br s, 1H), 2.63 (br d, J = 3.6 Hz, 1H), 2.23 (br dd, J = 4.4, 12.8 Hz, 1H), 2.17-2.08 (m, 1H). MS: (ESI) m/z calculated for C₂₁H₁₅FN₂O₃ [M + H]⁺ 363.1, found 363.2. |
| 261 | 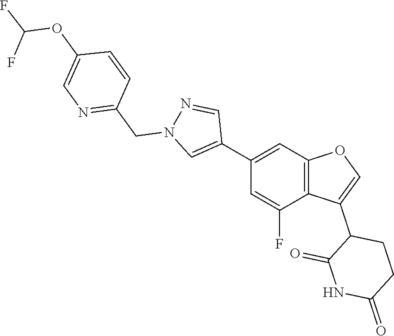 [SMILES: O=C1CCC(c2coc3cc(-c4cnn(Cc5ccc(OC(F)F)cn5)c4)cc(F)c23)C(=O)N1] | ¹H NMR (400 MHz, DMSO-d₆) δ 10.96-10.89 (m, 1H), 8.47-8.42 (m, 2H), 8.07 (s, 1H), 7.91 (s, 1H), 7.75-7.65 (m, 2H), 7.50-7.10 (m, 3H), 5.45 (s, 2H), 4.20-4.08 (m, 1H), 2.84-2.71 (m, 1H), 2.65- 2.54 (m, 1H), 2.25-2.15 (m, 1H), 2.13-2.05 (m, 1H). MS: (ESI) m/z calculated for C₂₃H₁₇F₃N₄O₄ [M + H]⁺ 471.1, found 471.2. |
| 262 | 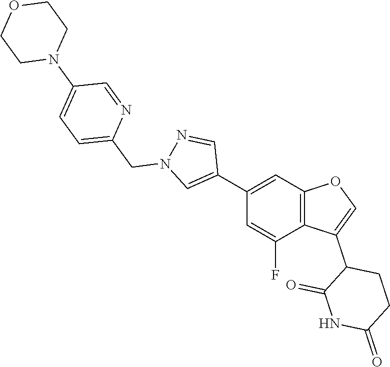 [SMILES: O=C1CCC(c2coc3cc(-c4cnn(Cc5ccc(N6CCOCC6)cn5)c4)cc(F)c23)C(=O)N1] | ¹H NMR (400 MHz, DMSO-d₆) δ 10.92 (s, 1H), 8.39-8.35 (m, 1H), 8.25 (d, J = 2.8 Hz, 1H), 8.03 (s, 1H), 7.90 (s, 1H), 7.72 (s, 1H), 7.38-7.31 (m, 2H), 7.11 (d, J = 8.8 Hz, 1H), 5.31 (s, 2H), 4.13 (dd, J = 5.2, 12.4 Hz, 1H), 3.75-3.71 (m, 4H), 3.16- 3.13 (m, 4H), 2.67 (s, 2H), 2.61 (s, 1H), 2.33 (s, 1H). |
| 263 | 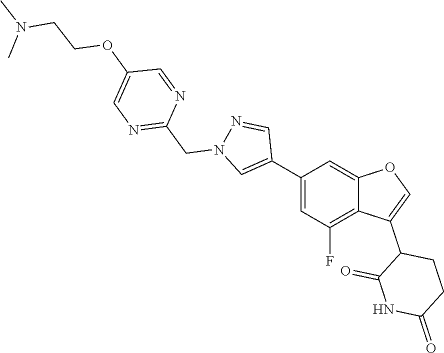 [SMILES: CN(C)CCOc1cnc(Cn2cc(-c3cc(F)c4c(C5CCC(=O)NC5=O)coc4c3)cn2)nc1] | ¹H NMR (400 MHz, DMSO-d₆) δ 10.91 (s, 1H), 8.54 (s, 2H), 8.39 (s, 1H), 8.26 (s, 1H), 8.01 (s, 1H), 7.91 (s, 1H), 7.72 (s, 1H), 7.36 (d, J = 11.6 Hz, 1H), 5.48 (s, 2H), 4.21 (t, J = 5.6 Hz, 2H), 4.16-4.11 (m, 1H), 2.84-2.74 (m, 1H), 2.64-2.61 (m, 2H), 2.56 (d, J = 1.6 Hz, 1H), 2.27-2.21 (m, 1H), 2.20 (s, 6H), 2.13-2.07 (m, 1H). MS: (ESI) m/z calculated for C₂₅H₂₅FN₆O₄ [M + H]⁺ 493.2, found 493.2. |
| 264 | 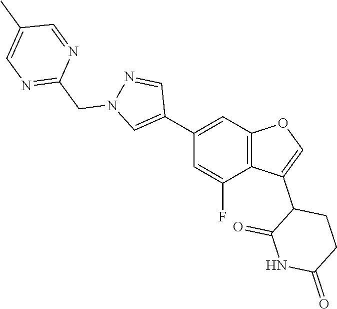 [SMILES: Cc1cnc(Cn2cc(-c3cc(F)c4c(C5CCC(=O)NC5=O)coc4c3)cn2)nc1] | ¹H NMR (400 MHz, DMSO-d₆) δ 10.95 (s, 1H), 8.64 (s, 2H), 8.41 (s, 1H), 8.03 (s, 1H), 7.92 (s, 1H), 7.74 (s, 1H), 7.36 (s, 1H), 5.51 (s, 2H), 4.15 (dd, J = 12.0, 4.8 Hz, 1H), 2.91-2.69 (m, 1H), 2.60 (d, J = 12.0 Hz, 1H), 2.26 (s, 3H), 2.25-2.17 (m, 1H), 2.14-2.05 (m, 1H), 1.28-1.13 (m, 1H). |
| 265 | 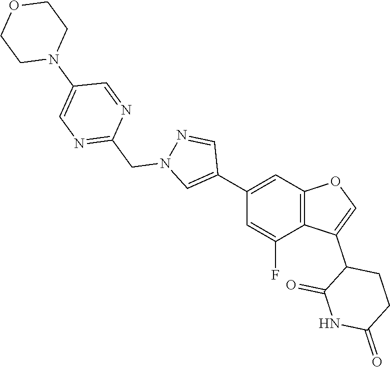 [SMILES: O=C1CCC(c2coc3cc(-c4cnn(Cc5ncc(N6CCOCC6)cn5)c4)cc(F)c23)C(=O)N1] | ¹H NMR (400 MHz, DMSO-d₆) δ 10.96-10.88 (m, 1H), 8.47 (s, 2H), 8.39-8.34 (m, 1H), 8.03-7.97 (m, 1H), 7.94-7.87 (m, 1H), 7.76-7.68 (m, 1H), 7.41-7.31 (m, 1H), 5.44-5.39 (m, 2H), 4.14 (dd, J = 4.0, 12.0 Hz, 1H), 3.77-3.71 (m, 4H), 3.24-3.20 (m, 4H), 2.83 (br d, J = 5.2 Hz, 1H), 2.62-2.57 (m, 1H), 2.24-2.15 (m, 1H), 2.13-2.06 (m, 1H). ¹⁹F NMR (376 MHz, DMSO-d₆) δ −119.14. MS: (ESI) m/z calculated for C₂₅H₂₃FN₆O₄ [M + H]⁺ 491.1, found 491.2. |

### Example 132: 3-[6-(6-methyl-1H-indol-2-yl) furo[2,3-b]pyridin-3-yl]piperidine-2,6-dione

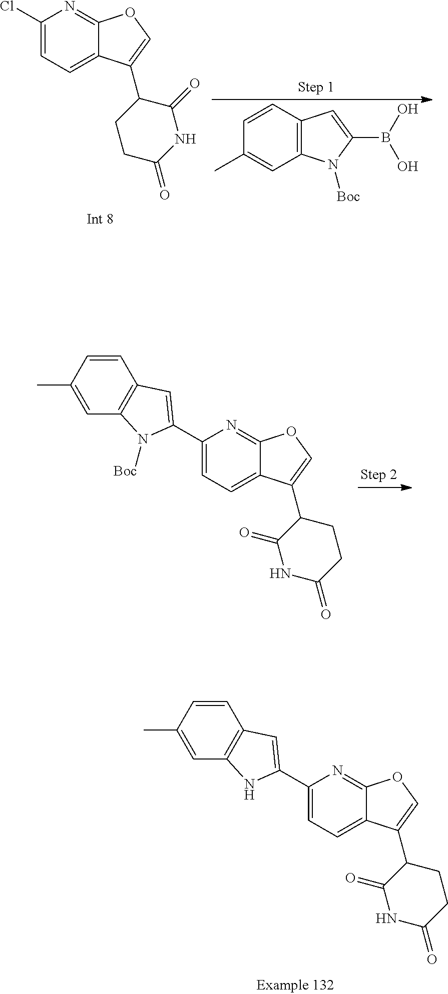 [SMILES: O=C1CCC(c2coc3nc(Cl)ccc23)C(=O)N1 ; Cc1ccc2cc(B(O)O)n(C(=O)OC(C)(C)C)c2c1 ; Cc1ccc2cc(-c3ccc4c(C5CCC(=O)NC5=O)coc4n3)n(C(=O)OC(C)(C)C)c2c1 ; Cc1ccc2cc(-c3ccc4c(C5CCC(=O)NC5=O)coc4n3)[nH]c2c1]

Step 1: A mixture of Int 8 (19.85 mg, 75.00 μmol, 1 eq), (1-tert-butoxycarbonyl-6-methyl-indol-2-yl) boronic acid (20.63 mg, 75.00 μmol, 1 eq), XantPhos Pd G3 (7.11 mg, 7.50 μmol, 0.1 eq) and K₃PO₄ (23.88 mg, 112.49 μmol, 1.5 eq) in dioxane (2 mL) and H₂O (0.5 mL) was degassed and purged with N₂ for 3 times, and then the mixture was stirred at 80° C. for 1 h under N₂ atmosphere. The reaction mixture was concentrated. Water (50 mL) was added to the residue. The resulting mixture was extracted with EtOAc (30 mL×3). The combined organic phase was washed with brine (50 mL), dried over anhydrous Na₂SO₄, filtered and concentrated. The residue was purified by prep-HPLC to give crude tert-butyl-2-[3-(2,6-dioxo-3-piperidyl) furo[2,3-b]pyridin-6-yl]-6-methyl-indole-1-carboxylate (5.5 mg, 15.90% yield) as a white solid.

Step 2: To a solution of crude tert-butyl 2-[3-(2,6-dioxo-3-piperidyl) furo[2,3-b]pyridin-6-yl]-6-methyl-indole-1-carboxylate (80 mg, 85.31 μmol, 49% purity, 1 eq) in HCl/dioxane (2 mL). The mixture was stirred at 20° C. for 1 h. The reaction mixture was concentrated. The residue was purified by prep-HPLC to give Example 132 (8.5 mg, 27.19% yield) as a yellow solid. ¹H NMR (400 MHZ, DMSO-d₆) δ 11.56 (s, 1H), 10.95 (s, 1H), 8.12 (d, J=8.0 Hz, 1H), 8.02 (s, 1H), 7.94 (d, J=8.0 Hz, 1H), 7.44 (d, J=8.4 Hz, 1H), 7.26 (s, 1H), 7.14 (d, J=1.6 Hz, 1H), 6.85 (d, J=8.4 Hz, 1H), 4.21-4.13 (dd, J=4.8, 12.0 Hz, 1H), 2.81-2.71 (m, 1H), 2.65-2.57 (m, 1H), 2.40 (s, 3H), 2.37-2.31 (m, 1H), 2.20-2.11 (m, 1H). LCMS: calc. for C₂₁H₁₇N₃O₃: 359.1, found: [M+H]⁺360.1.

Examples 276-281 were prepared following the similar procedure that was used for preparing Examples 132, starting from Int-8 and their corresponding boronic acid or ester intermediates, with proper modifications. Characterizations of Examples 276-281 are provided in Table 7.

TABLE 7

| Example | | |
|---|---|---|
| ID | Structure | NMR/MS |
| 276 | 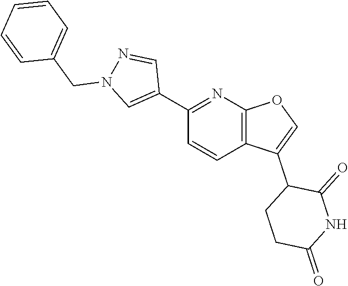 [SMILES: O=C1CCC(c2coc3nc(-c4cnn(Cc5ccccc5)c4)ccc23)C(=O)N1] | ¹H NMR (400 MHz, DMSO-d₆) δ 10.92 (s, 1H), 8.47 (s, 1H), 8.08 (s, 1H), 8.04 (d, J = 8.4 Hz, 1H), 7.93 (s, 1H), 7.64 (d, J = 8.0 Hz, 1H), 7.36- 7.30 (m, 5H), 5.37 (s, 2H), 4.15-4.10 (m, 1H), 2.74-2.61 (m, 1H), 2.60-2.56 (m, 1H), 2.36- 2.34 (m, 1H), 2.15-2.14 (m, 1H). MS: (ESI) m/z [M + H]⁺ 387.2 |
| 277 | 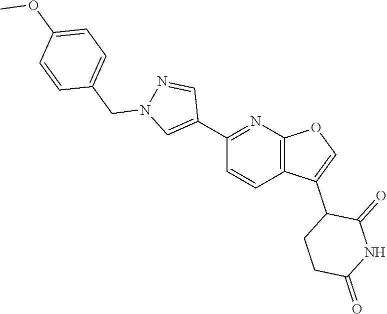 [SMILES: COc1ccc(Cn2cc(-c3ccc4c(C5CCC(=O)NC5=O)coc4n3)cn2)cc1] | ¹H NMR (400 MHz, DMSO-d₆) δ 10.91 (s, 1H), 8.40 (s, 1H), 8.06-8.02 (m, 2H), 7.93 (s, 1H), 7.62 (d, J = 8.0 Hz, 1H), 7.29 (d, J = 8.8 Hz, 2H), 6.92 (d, J = 8.8 Hz, 2H), 5.28 (s, 2H), 4.14-4.10 (m, 1H), 3.73 (s, 3H), 2.73-2.60 (m, 1H), 2.59- 2.56 (m, 1H), 2.33-2.32 (m, 1H), 2.14-2.13 (m, 1H). MS: (ESI) m/z [M + H]⁺ 417.2 |
| 278 | 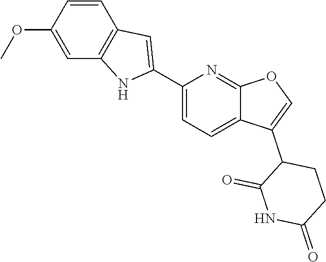 [SMILES: COc1ccc2cc(-c3ccc4c(C5CCC(=O)NC5=O)coc4n3)[nH]c2c1] | ¹H NMR (400 MHz, DMSO-d₆) δ 11.54 (s, 1H), 10.94 (s, 1H), 8.09 (d, J = 8.0 Hz, 1H), 8.00 (s, 1H), 7.89 (d, J = 8.0 Hz, 1H), 7.44 (d, J = 8.4 Hz, 1H), 7.12 (d, J = 1.6 Hz, 1H), 6.96 (s, 1H), 6.69- 6.66 (m, 1H), 4.18-4.17 (m, 1H), 3.77 (s, 3H), 2.76-2.75 (m, 1H), 2.74-2.72 (m, 1H), 2.40- 2.39 (m, 1H), 2.38-2.36 (m, 1H). MS: (ESI) m/z [M + H]⁺ 376.4 |
| 279 | 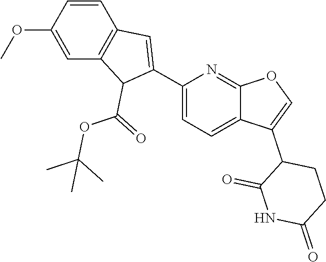 [SMILES: COc1ccc2c(c1)C(C(=O)OC(C)(C)C)C(c1ccc3c(C4CCC(=O)NC4=O)coc3n1)=C2] | ¹H NMR (400 MHz, DMSO-d₆) δ 10.94 (s, 1H), 8.15 (d, J = 8.0 Hz, 1H), 8.10 (s, 1H), 7.63 (d, J = 8.0 Hz, 1H), 7.55 (d, J = 8.8 Hz, 2H), 6.96-6.91 (m, 2H), 4.21-4.17 (m, 1H), 3.83 (s, 3H), 2.76- 2.73 (m, 1H), 2.62-2.61 (m, 1H), 2.40-2.37 (m, 1H), 2.17-2.16 (m, 1H), 1.23 (s, 9H). MS: (ESI) m/z [M + H]⁺ 476.4 |
| 280 | 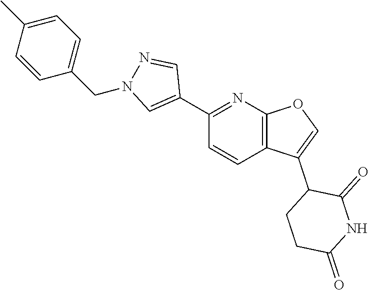 [SMILES: Cc1ccc(Cn2cc(-c3ccc4c(C5CCC(=O)NC5=O)coc4n3)cn2)cc1] | ¹H NMR (400 MHz, DMSO-d₆) δ 10.92 (s, 1H), 8.43 (s, 1H), 8.07 (s, 1H), 8.04 (d, J = 8.0 Hz, 1H), 7.95-7.92 (m, 1H), 7.65-7.62 (m, 1H), 7.27-7.10 (m, 4H), 5.32 (s, 2H), 4.17-4.09 (m, 2H), 2.80-2.68 (m, 1H), 2.63-2.55 (m, 1H), 2.41-2.32 (m, 1H), 2.28 (s, 3H), 2.17-2.09 (m, 1H). MS: (ESI) m/z [M + H]⁺ 401.2 |
| 281 | 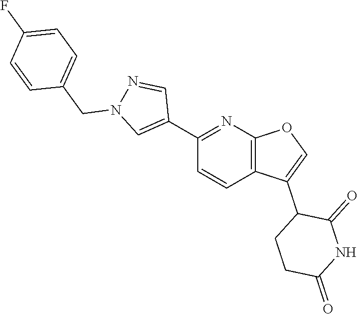 [SMILES: O=C1CCC(c2coc3nc(-c4cnn(Cc5ccc(F)cc5)c4)ccc23)C(=O)N1] | ¹H NMR (400 MHz, DMSO-d₆) δ 10.93 (s, 1H), 8.48 (s, 1H), 8.09 (s, 1H), 8.05 (d, J = 8.0 Hz, 1H), 7.94 (s, 1H), 7.64 (d, J = 8.0 Hz, 1H), 7.38 (dd, J = 5.6, 8.4 Hz, 2H), 7.20 (t, J = 8.8 Hz, 2H), 5.44-5.28 (m, 2H), 4.13 (dd, J = 4.4, 12.4 Hz, 1H), 2.75-2.66 (m, 1H), 2.63-2.56 (m, 1H), 2.37-2.31 (m, 1H), 2.17-2.09 (m, 1H). ¹⁹F NMR (376 MHz, DMSO-d₆) δ −114.72. MS: (ESI) m/z [M + H]⁺ 405. |

### Example 266:3-(4-fluoro-6-(1-((5-morpholinopyrimidin-2-yl)methyl)-1H-pyrazol 4-yl)benzo[d]isoxazol-3-yl)piperidine-2,6-dione

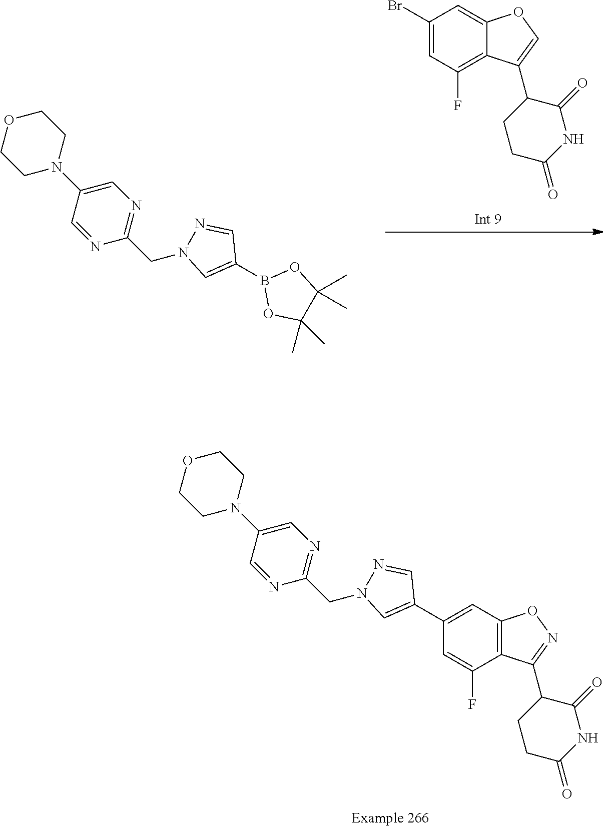 [SMILES: CC1(C)OB(c2cnn(Cc3ncc(N4CCOCC4)cn3)c2)OC1(C)C ; O=C1CCC(c2coc3cc(Br)cc(F)c23)C(=O)N1 ; O=C1CCC(c2noc3cc(-c4cnn(Cc5ncc(N6CCOCC6)cn5)c4)cc(F)c23)C(=O)N1]

A mixture of 4-[2-[4-(4,4,5,5-tetramethyl-1,3,2-dioxaborolan-2-yl)pyrazol-1-yl]methyl]pyrimidin-5-yl]morpholine (45 mg, 121.22 μmol, 1 eq), Int 9 (39.65 mg, 121.22 μmol, 1 eq), Xphos Pd G3 (10.26 mg, 12.12 μmol, 0.1 eq), K₃PO₄ (51.46 mg, 242.43 μmol, 2.0 eq) in dioxane (2 mL) and H₂O (0.5 mL) was degassed and purged with N₂ for 3 times, and then the mixture was stirred at 80° C. for 1 h under N₂ atmosphere. The reaction mixture was concentrated directly. The residue was purified by prep-HPLC to give Example 266 (2.0 mg, 3.26% yield) as a white solid. ¹H NMR (400 MHZ, DMSO-d₆) § 11.23-11.10 (m, 1H), 8.52 (s, 1H), 8.48 (s, 2H), 8.12 (s, 1H), 7.90 (s, 1H), 7.62-7.50 (m, 1H), 5.44 (s, 2H), 4.66-4.50 (m, 1H), 3.73 (d, J=8.0 Hz, 4H), 3.25-3.18 (m, 4H), 2.90-2.79 (m, 1H), 2.66 (d, J=4.0 Hz, 1H), 2.33 (d, J=2.0 Hz, 1H), 2.25-2.17 (m, 1H). 1° F. NMR (376 MHz, DMSO-d₆)₈-113.899. LCMS: calc. for C₂₄H₂₂FN₇O₄:491.2, found: [M+H]⁺492.1.

Examples 267-270 were prepared following the similar procedure that was used for preparing Examples 266, starting from Int-9 and their corresponding boronic acid or ester intermediates, with proper modifications. Characterizations of Examples 267-270 are provided in Table 8.

TABLE 8

| Example | | |
|---|---|---|
| ID | Structure | NMR/MS |
| 267 | 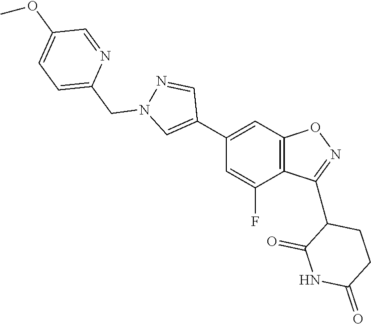 [SMILES: COc1ccc(Cn2cc(-c3cc(F)c4c(C5CCC(=O)NC5=O)noc4c3)cn2)nc1] | ¹H NMR (400 MHz, DMSO-d₆) δ 11.20 (s, 1H), 8.52 (s, 1H), 8.25 (d, J = 2.8 Hz, 1H), 8.15 (s, 1H), 7.89 (s, 1H), 7.55 (d, J = 11.2 Hz, 1H), 7.40 (dd, J = 2.8, 8.4 Hz, 1H), 7.23 (d, J = 8.4 Hz, 1H), 5.38 (s, 2H), 4.58 (dd, J = 5.2, 12.4 Hz, 1H), 3.81 (s, 3H), 2.85 (ddd, J = 5.6, 12.4, 17.6 Hz, 1H), 2.68-2.63 (m, 1H), 2.31-2.25 (m, 1H), 2.25-2.17 (m, 1H). MS: (ESI) m/z calculated for C₂₂H₁₈N₅O₄ [M + H]⁺ 436.1, found 436.1. |
| 268 | 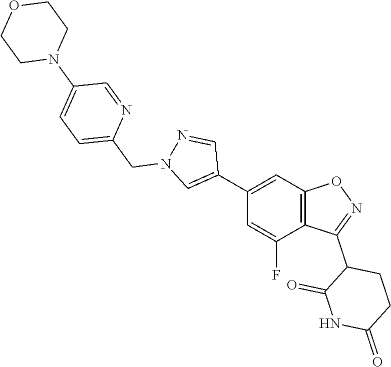 [SMILES: O=C1CCC(c2noc3cc(-c4cnn(Cc5ccc(N6CCOCC6)cn5)c4)cc(F)c23)C(=O)N1] | ¹H NMR (400 MHz, DMSO-d₆) δ 11.23 (s, 1H), 8.51 (s, 1H), 8.25 (d, J = 2.8 Hz, 1H), 8.14 (s, 1H), 7.55 (d, J = 11.6 Hz, 1H), 7.34-7.32 (m, 1H), 7.14 (d, J = 8.8 Hz, 1H), 5.33 (s, 2H), 4.60- 4.55 (m, 1H), 3.71-3.70 (m, 1H), 3.75-3.70 (m, 4H), 3.16-3.13 (m, 4H), 2.89-2.80 (m, 1H), 2.67-2.59 (m, 1H), 2.33-2.29 (m, 1H), 2.25- 2.17 (m, 1H). ¹⁹F NMR (376 MHz, DMSO-d₆) δ −113.92. MS: (ESI) m/z calculated for C₂₅H₂₃FN₆O₄ [M + H]⁺ 491.1, found 491.1. |
| 269 | 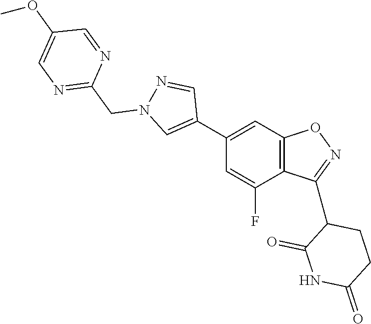 [SMILES: COc1cnc(Cn2cc(-c3cc(F)c4c(C5CCC(=O)NC5=O)noc4c3)cn2)nc1] | ¹H NMR (400 MHz, DMSO-d₆) δ 11.17 (br s, 1H), 8.57-8.49 (m, 3H), 8.13 (s, 1H), 7.90 (s, 1H), 7.56 (d, J = 11.6 Hz, 1H), 5.51 (s, 2H), 4.64- 4.54 (m, 1H), 3.90 (s, 3H), 2.84 (br d, J = 12.4 Hz, 1H), 2.69-2.64 (m, 1H), 2.33 (br s, 1H), 2.23 (br d, J = 2.8 Hz, 1H). ¹⁹F NMR (376 MHz, DMSO-d₆) δ −113.88. MS: (ESI) m/z calculated for C₂₁H₁₇FN₆O₄ [M + H]⁺ 437.1, found 437.1. |
| 270 | 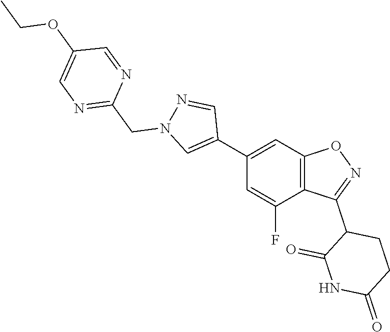 [SMILES: CCOc1cnc(Cn2cc(-c3cc(F)c4c(C5CCC(=O)NC5=O)noc4c3)cn2)nc1] | ¹H NMR (400 MHz, DMSO-d₆) δ 11.20-11.14 (m, 1H), 8.56-8.51 (m, 3H), 8.13 (s, 1H), 7.93 (s, 1H), 7.60-7.51 (m, 1H), 5.50 (s, 2H), 4.63- 4.55 (m, 1H), 4.22-4.15 (m, 2H), 2.91-2.79 (m, 1H), 2.65-2.59 (m, 1H), 2.25-2.19 (m, 1H), 1.35 (t, J = 6.8 Hz, 3H). ¹⁹F NMR (376 MHz, DMSO-d₆) δ −113.90. MS: (ESI) m/z calculated for C₂₂H₁₉FN₆O₄ [M + H]⁺ 451.1, found 451.2. |

### Example 271:3-(5-chloro-6-(1-((5-morpholinopyridin-2-yl)methyl)-1H-pyrazol-4-yl)benzofuran-3-yl)piperidine-2,6-dione

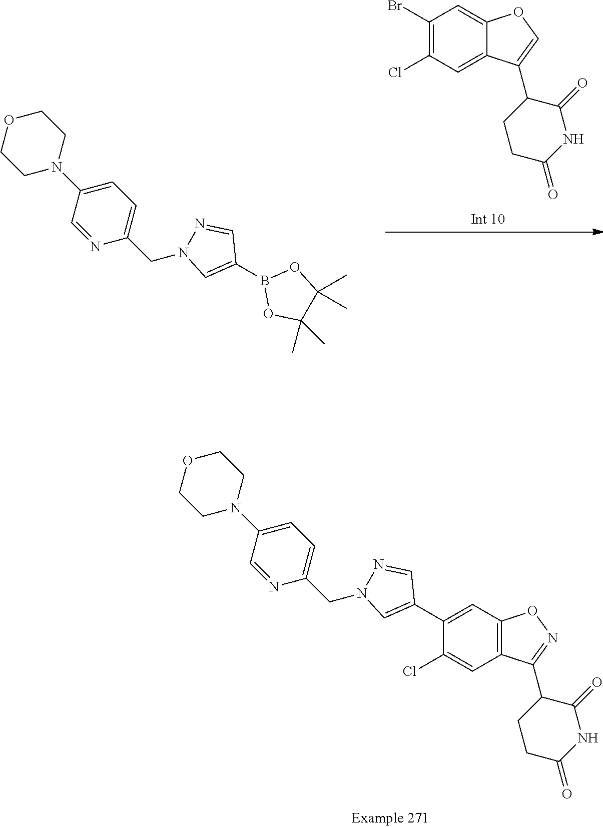 [SMILES: CC1(C)OB(c2cnn(Cc3ccc(N4CCOCC4)cn3)c2)OC1(C)C ; O=C1CCC(c2coc3cc(Br)c(Cl)cc23)C(=O)N1 ; O=C1CCC(c2noc3cc(-c4cnn(Cc5ccc(N6CCOCC6)cn5)c4)c(Cl)cc23)C(=O)N1]

A mixture of 4-[6-[[4-(4,4,5,5-tetramethyl-1,3,2-dioxaborolan-2-yl)pyrazol-1-yl]methyl]-3-pyridyl]morpholine (140 mg, 378.12 μmol, 1 eq), Int 10 (129.5 mg, 378.12 μmol, 1 eq), XPhos Pd G3 (32.0 mg, 37.81 μmol, 0.1 eq), K₃PO₄ (120.4 mg, 567.18 μmol, 1.5 eq) in dioxane (3 mL) and H₂O (1 mL) was degassed and purged with N₂ for 3 times, and then the mixture was stirred at 80° C. for 0.5 h under N₂ atmosphere. H₂O (10 mL) was added to the residue. The resulting mixture was extracted with EtOAc (10 mL×3). The combined organic phase was washed with brine (10 mL), dried over anhydrous Na₂SO₄, filtered and concentrated. The residue was purified by prep-HPLC to give Example 271 (4.6 mg, 2.36% yield) as a white solid. ¹H NMR (400 MHZ, DMSO-d₆) § 10.91 (s, 1H), 8.49 (s, 1H), 8.31 (s, 1H), 8.26 (d, J=2.4 Hz, 1H), 7.96 (s, 1H), 7.90 (s, 1H), 7.81 (s, 1H), 7.78 (s, 1H), 7.35-7.32 (m, 1H), 7.12 (d, J=8.6 Hz, 1H), 5.37 (s, 2H), 4.17-4.13 (m, 1H), 3.76-3.71 (m, 4H), 3.17 -3.13 (m, 4H), 2.70-2.66 (m, 1H), 2.39-2.32 (m, 1H), 2.14-2.07 (m, 1H). LCMS: calc. for C₂₅H₂₃ClN₆O₄:505.1, found: [M+H]⁺506.1.

Examples 272-275 were prepared following the similar procedure that was used for preparing Examples 271, starting from Int-10 and their corresponding boronic acid or ester intermediates, with proper modifications. Characterizations of Examples 272-275 are provided in Table 9.

TABLE 9

| Example | | |
|---|---|---|
| ID | Structure | NMR/MS |
| 272 | 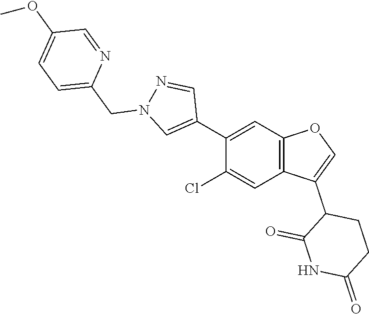 [SMILES: COc1ccc(Cn2cc(-c3cc4occ(C5CCC(=O)NC5=O)c4cc3Cl)cn2)nc1] | ¹H NMR (400 MHz, DMSO-d6) δ 10.90 (s, 1H), 8.32 (s, 1H), 8.25 (s, 1H), 7.96 (s, 1H), 7.91 (s, 1H), 7.81 (s, 1H), 7.78 (s, 1H), 7.41-7.38 (m, 2H), 7.21-7.19 (d, J = 4.0 Hz, 1H), 5.42 (s, 2H), 4.17-4.13 (m, 1H), 3.82 (s, 3H), 2.73-2.70 (m, 1H), 2.60-2.35 (m, 1H), 2.34-2.32 (m, 1H), 2.13-2.11 (m, 1H). MS: (ESI) m/z calculated for C23H19ClN4O4 [M + H]⁺ 451.1, found 450.9. |
| 273 | 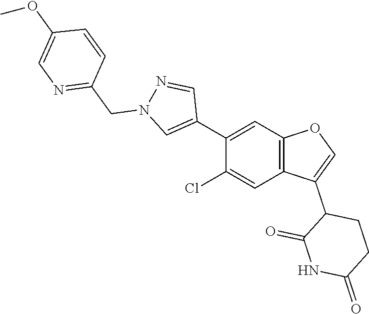 [SMILES: COc1ccc(Cn2cc(-c3cc4occ(C5CCC(=O)NC5=O)c4cc3Cl)cn2)nc1] | ¹H NMR (400 MHz, DMSO-d₆) δ 10.91 (s, 1H), 8.55 (s, 2H), 8.34 (s, 1H), 7.97 (s, 1H), 7.90 (s, 1H), 7.83 (s, 1H), 7.79 (s, 1H), 5.54 (s, 2H), 4.20-4.09 (m, 1H), 3.90 (s, 3H), 2.78-2.69 (m, 1H), 2.60 (d, J = 4.0 Hz, 1H), 2.42-2.34 (m, 1H), 2.12 (s, 1H). MS: (ESI) m/z calculated for C₂₂H₁₈ClN₅O₄ [M + H]⁺ 452.1, found 452.0. |
| 274 | 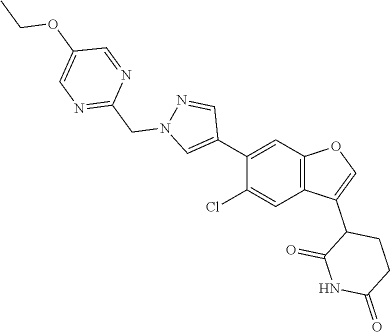 [SMILES: CCOc1cnc(Cn2cc(-c3cc4occ(C5CCC(=O)NC5=O)c4cc3Cl)cn2)nc1] | ¹H NMR (400 MHz, DMSO-d₆) δ 10.91 (s, 1H), 8.53 (s, 2H), 8.34 (s, 1H), 7.96 (s, 1H), 7.90 (s, 1H), 7.83 (s, 1H), 7.79 (s, 1H), 5.53 (s, 2H), 4.21-4.16 (m, 2H), 2.75-2.66 (m, 1H), 2.60-2.55 (m, 1H), 2.42-2.30 (m, 2H), 2.15-2.08 (m, 1H), 1.35 (t, J = 8.0 Hz, 3H). MS: (ESI) m/z calculated for C₂₃H₂₀ClN₅O₄ [M + H]⁺ 466.1, found 466.1. |
| 275 | 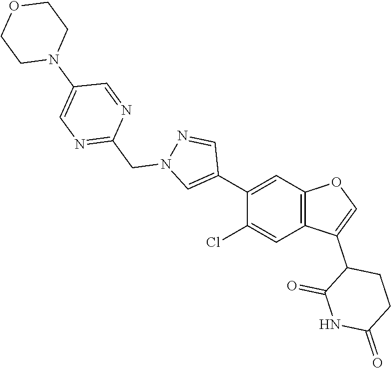 [SMILES: O=C1CCC(c2coc3cc(-c4cnn(Cc5ncc(N6CCOCC6)cn5)c4)c(Cl)cc23)C(=O)N1] | ¹H NMR (400 MHz, DMSO-d₆) δ 10.92 (s, 1H), 8.48 (s, 2H), 8.32 (s, 1H), 7.96 (s, 1H), 7.89 (s, 1H), 7.82 (s, 1H), 7.78 (s, 1H), 5.47 (s, 2H), 4.20-4.10 (m, 1H), 3.73 (d, J = 5.0 Hz, 4H), 3.25-3.20 (m, 4H), 2.73 (s, 1H), 2.62-2.55 (m, 1H), 2.39-2.32 (m, 1H), 2.15- 2.07 (m, 1H). MS: (ESI) m/z calculated for C₂₅H₂₃ClN₆O₄ [M + H]⁺ 507.1, found 507.1. |

### Example 282: 3-[6-[1-[(4-methoxyphenyl)methyl]pyrazol-4-yl]furo[3,2-b]pyridin-3-yl]piperidine-2,6-dione

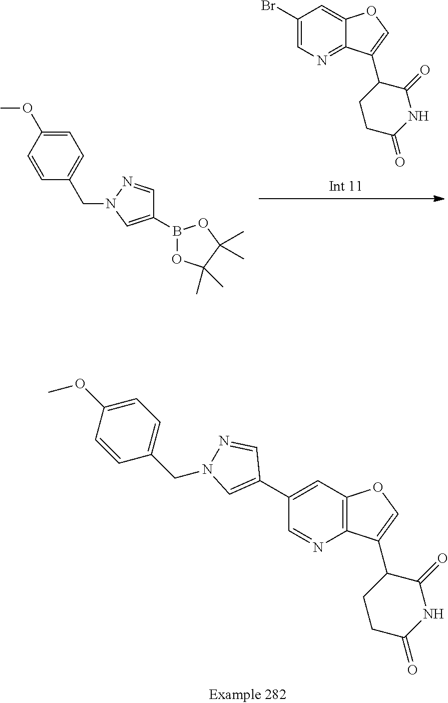

A mixture of Int 11 (30 mg, 97.05 μmol, 1 eq), 1-[(4-methoxyphenyl)methyl]-4-(4,4,5,5-tetramethyl-1,3,2-dioxaborolan-2-yl)pyrazole (33.54 mg, 106.76 μmol, 1.1 eq), K₃PO₄ (41.20 mg, 194.10 μmol, 2 eq), XPhos Pd G3 (8.21 mg, 9.71 μmol, 0.1 eq) in dioxane (3 mL) and H₂O (0.5 mL) was degassed and purged with N₂ for 3 times, and then the mixture was stirred at 80° C. for 2 h under N₂ atmosphere. The reaction mixture was concentrated directly. The residue was purified by prep-HPLC to give Example 282 (2.1 mg, 5.04 μmol, 5.20% yield) as a white solid. ¹H NMR (400 MHz, DMSO-d₆) δ 10.92 (s, 1H), 8.76 (s, 1H), 8.39 (s, 1H), 8.20 (d, J=12.4 Hz, 2H), 8.04 (s, 1H), 7.27 (br d, J=8.4 Hz, 2H), 6.91 (br d, J=8.4 Hz, 2H), 5.28 (s, 2H), 4.14 (dd, J=4.8, 11.2 Hz, 1H), 3.73 (s, 3H), 2.82-2.70 (m, 1H), 2.63-2.56 (m, 1H), 2.44-2.39 (m, 1H), 2.20-2.10 (m, 1H). MS: (ESI) m/z calculated for C₂₃H₂ON₄O₄ [M+H]⁺417.2, found 417.3.

Examples 283-287 were prepared following the similar procedure that was used for preparing Example 282, starting from Int-11 and their corresponding boronic acid or ester intermediates, with proper modifications. Characterizations of Examples 283-287 are provided in Table 10.

TABLE 10

| Example | | |
|---|---|---|
| ID | Structure | NMR/MS |
| 283 | 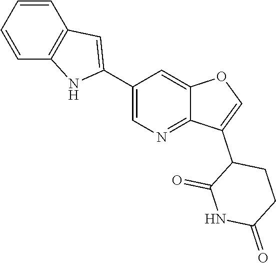 [SMILES: O=C1CCC(c2coc3cc(-c4cc5ccccc5[nH]4)cnc23)C(=O)N1] | ¹H NMR (400 MHz, DMSO-d₆) δ 11.72 (s, 1H), 10.95 (s, 1H), 9.05 (s, 1H), 8.45 (s, 1H), 8.28 (s, 1H), 7.57- 7.42 (m, 2H), 7.13-7.01 (m, 3H), 4.21-4.17 (m, 1H), 2.78-2.64 (m, 1H), 2.63-2.62 (m, 2H), 2.20-2.19 (m, 1H). MS: (ESI) m/z [M + H]⁺ 346.1 |
| 284 | 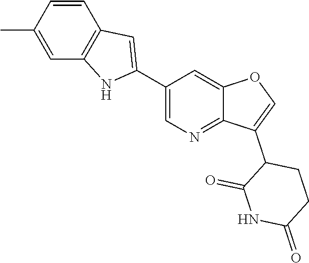 [SMILES: Cc1ccc2cc(-c3cnc4c(C5CCC(=O)NC5=O)coc4c3)[nH]c2c1] | ¹H NMR (400 MHz, DMSO-d₆) δ 11.51 (s, 1H), 10.95 (s, 1H), 9.03 (d, J = 1.6 Hz, 1H), 8.41 (d, J = 1.6 Hz, 1H), 8.30-8.12 (m, 1H), 7.44 (d, J = 8.0 Hz, 1H), 7.21 (s, 1H), 7.02 (s, 1H), 6.86 (d, J = 8.0 Hz, 1H), 4.18 (dd, J = 4.8, 12.0 Hz, 1H), 2.85-2.72 (m, 1H), 2.68-2.55 (m, 1H), 2.47 (br d, J = 4.0 Hz, 1H), 2.41 (s, 3H), 2.25-2.12 (m, 1H). MS: (ESI) m/z [M + H]⁺ 360.3 |
| 285 | 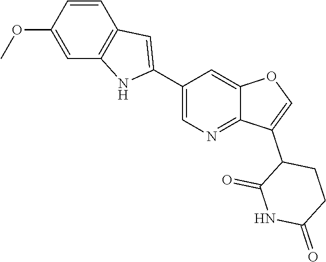 [SMILES: COc1ccc2cc(-c3cnc4c(C5CCC(=O)NC5=O)coc4c3)[nH]c2c1] | ¹H NMR (400 MHz, DMSO-d₆) δ 11.52 (s, 1H), 10.98- 10.90 (m, 1H), 8.99 (d, J = 2.0 Hz, 1H), 8.36 (d, J = 2.0 Hz, 1H), 8.25 (s, 1H), 7.47-7.39 (m, 1H), 7.00 (d, J = 1.6 Hz, 1H), 6.90 (d, J = 2.0 Hz, 1H), 6.74-6.66 (m, 1H), 4.18 (dd, J = 5.2, 12.0 Hz, 1H), 3.80 (s, 3H), 2.86-2.73 (m, 1H), 2.65-2.57 (m, 1H), 2.46-2.31 (m, 1H), 2.24-2.13 (m, 1H). MS: (ESI) m/z [M + H]⁺ 376.3 |
| 286 | 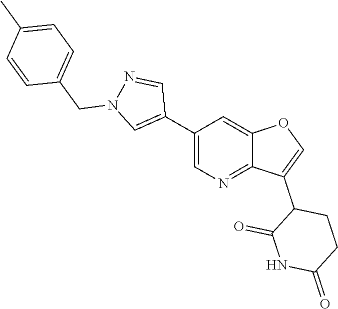 [SMILES: Cc1ccc(Cn2cc(-c3cnc4c(C5CCC(=O)NC5=O)coc4c3)cn2)cc1] | ¹H NMR (400 MHz, DMSO-d₆) δ 11.02-10.75 (m, 1H), 8.80-8.75 (m, 1H), 8.44-8.36 (m, 1H), 8.24- 8.17 (m, 2H), 8.08-8.04 (m, 1H), 7.23-7.13 (m, 4H), 5.31 (s, 2H), 4.19-4.09 (m, 1H), 2.79-2.71 (m, 1H), 2.62-2.56 (m, 1H), 2.45 (br dd, J = 4.0, 12.4 Hz, 1H), 2.28 (s, 3H), 2.20-2.11(m, 1H). MS: (ESI) m/z [M + H]⁺ 401.2 |
| 287 | 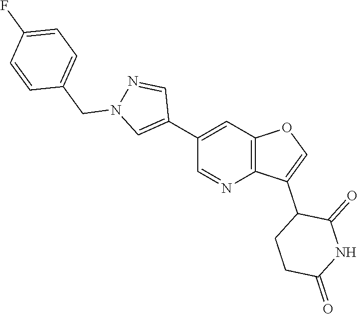 [SMILES: O=C1CCC(c2coc3cc(-c4cnn(Cc5ccc(F)cc5)c4)cnc23)C(=O)N1] | ¹H NMR (400 MHz, DMSO-d₆) δ 10.91 (s, 1H), 8.77 (d, J = 1.6 Hz, 1H), 8.43 (s, 1H), 8.22 (d, J = 1.6 Hz, 1H), 8.21-8.17 (m, 1H), 8.07 (s, 1H), 7.35 (s, 2H), 7.19 (s, 2H), 5.36 (s, 2H), 4.15 (dd, J = 4.8, 11.6 Hz, 1H), 2.61 (t, J = 4.0, 1H), 2.59-2.55 (m, 1H), 2.45 (dd, J = 3.6, 12.0 Hz, 1H), 2.16 (td, J = 4.4 8.8 Hz, 1H). ¹⁹F NMR (376 MHz, d₆-DMSO) δ −114.7. MS: (ESI) m/z [M + H]⁺ 405.3 |

### Example 288:3-(6-(1H-indol-2-yl) furo[3,2-c]pyridin-3-yl)piperidine-2,6-dione

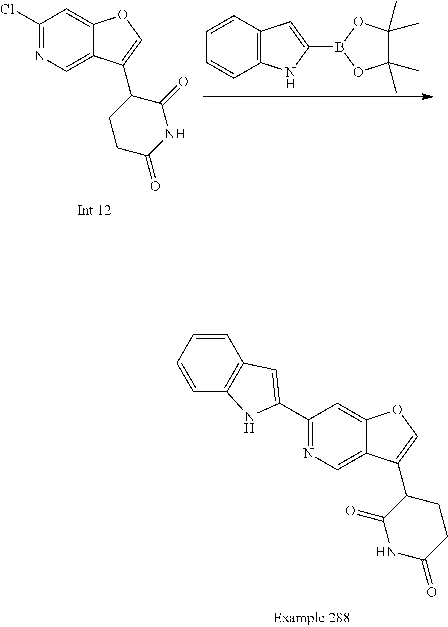

A mixture of Int 12 (20 mg, 60.45 μmol, 80% purity, 1 eq), 2-(4,4,5,5-tetramethyl-1,3,2-dioxaborolan-2-yl)-1H-indole (16.17 mg, 66.50 μmol, 1.1 eq), K₃PO₄ (19.25 mg, 90.68 μmol, 1.5 eq), XantPhos Pd G3 (5.73 mg, 6.05 μmol, 0.1 eq) in dioxane (2 mL) and H₂O (0.5 mL) was degassed and purged with N₂ for 3 times, and then the mixture was stirred at 80° C. for 2 h under N₂ atmosphere. The reaction mixture was concentrated. The residue was purified by prep-HPLC to give Example 288 (2.2 mg, 6.24 μmol, 10.32% yield, 97.9% purity) as a white solid. ¹H NMR (400 MHZ, DMSO-d₆) δ 11.66 (s, 1H), 10.97 (br s, 1H), 8.95 (s, 1H), 8.26 (s, 1H), 8.04 (s, 1H), 7.56 (d, J=8.0 Hz, 1H), 7.47 (d, J=8.0 Hz, 1H), 7.20 (d, J=1.2 Hz, 1H), 7.11 (t, J=6.8 Hz, 1H), 7.05-6.96 (m, 1H), 4.27 (dd, J=4.4, 12 Hz, 1H), 2.84-2.72 (m, 1H), 2.69-2.60 (m, 1H), 2.43-2.31 (m, 1H), 2.25-2.16 (m, 1H). MS: (ESI) m/z calculated for C₂₅H₂₃N₃O₅ [M+H]⁺346.1, found 346.3.

Examples 289-293 were prepared following the similar procedure that was used for preparing Example 288, starting from Int-12 and their corresponding boronic acid or ester intermediates, with proper modifications. Characterizations of Examples 289-293 are provided in Table 11.

TABLE 11

| Example | | |
|---|---|---|
| ID | Structure | NMR/MS |
| 289 | 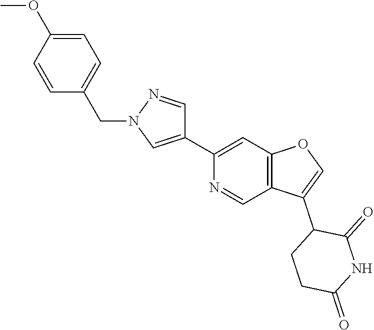 [SMILES: COc1ccc(Cn2cc(-c3cc4occ(C5CCC(=O)NC5=O)c4cn3)cn2)cc1] | ¹H NMR (400 MHz, DMSO-d₆) δ 10.95 (br s, 1H), 8.80 (s, 1H), 8.37 (s, 1H), 8.08 (s, 1H), 7.93 (d, J = 8.0 Hz, 2H), 7.28 (d, J = 8.4 Hz, 2H), 6.95-6.89 (m, 2H), 5.28 (s, 2H), 4.21 (dd, J = 4.8, 12.4 Hz, 1H), 3.73 (s, 3H), 2.81-2.70 (m, 1H), 2.65-2.56 (m, 1H), 2.37 (dq, J = 4.4, 12.8 Hz, 1H), 2.21-2.12 (m, 1H). MS: (ESI) m/z [M + H]⁺ 417.3 |
| 290 | 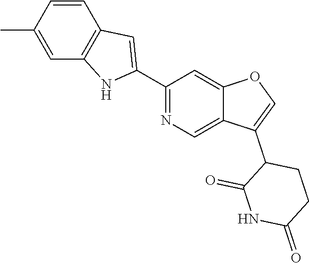 [SMILES: Cc1ccc2cc(-c3cc4occ(C5CCC(=O)NC5=O)c4cn3)[nH]c2c1] | ¹H NMR (400 MHz, DMSO-d₆) δ 11.80-11.62 (m, 1H), 11.01 (s, 1H), 9.02 (s, 1H), 8.35 (s, 1H), 8.13 (s, 1H), 7.48 (d, J = 8.0 Hz, 1H), 7.32-7.19 (m, 2H), 6.87 (br d, J = 8.0 Hz, 1H), 4.44-4.15 (m, 1H), 2.75 (dd, J = 5.6, 12.4 Hz, 1H), 2.65 (br s, 1H), 2.46-2.32 (m, 4H), 2.26-2.12 (m, 1H). MS: (ESI) m/z [M + H]⁺ 360.3 |
| 291 | 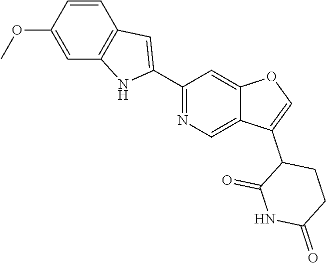 [SMILES: COc1ccc2cc(-c3cc4occ(C5CCC(=O)NC5=O)c4cn3)[nH]c2c1] | ¹1H NMR (400 MHz, DMSO-d₆) δ 11.51-11.45 (m, 1H), 8.91 (s, 1H), 8.17 (s, 1H), 8.02-7.99 (m, 1H), 7.43 (d, J = 8.6 Hz, 1H), 7.11 (d, J = 1.8 Hz, 1H), 6.97 (d, J = 1.8 Hz, 1H), 6.67 (dd, J = 2.3, 8.6 Hz, 1H), 4.26 (dd, J = 4.7, 12.2 Hz, 1H), 3.78 (s, 3H), 2.79-2.72 (m, 1H), 2.62 (td, J = 3.6, 17.4 Hz, 2H), 2.44-2.32 (m, 1H), 2.23-2.15 (m, 1H). MS: (ESI) m/z [M + H]⁺ 376.1 |
| 292 | 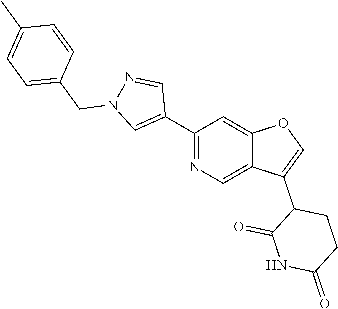 [SMILES: Cc1ccc(Cn2cc(-c3cc4occ(C5CCC(=O)NC5=O)c4cn3)cn2)cc1] | ¹H NMR (400 MHz, DMSO-d₆) δ 10.97-10.92 (m, 1H), 8.84-8.75 (m, 1H), 8.41-8.36 (m, 1H), 8.12- 8.05 (m, 1H), 7.93 (d, J = 7.4 Hz, 2H), 7.26-7.12 (m, 4H), 5.35-5.27 (m, 2H), 4.25-4.16 (m, 1H), 2.77-2.70 (m, 1H), 2.63-2.60 (m, 1H), 2.39-2.33 (m, 1H), 2.28 (s, 3H), 2.20-2.11 (m, 1H). MS: (ESI) m/z [M + H]⁺ 401.1 |
| 293 | 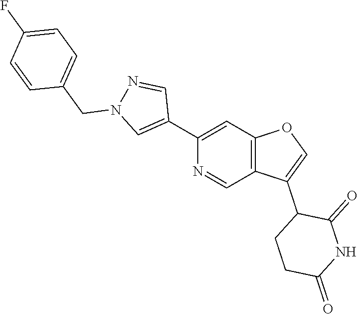 [SMILES: O=C1CCC(c2coc3cc(-c4cnn(Cc5ccc(F)cc5)c4)ncc23)C(=O)N1] | ¹H NMR (400 MHz, DMSO-d₆) δ 11.06-10.82 (m, 1H), 8.81 (d, J = 0.8 Hz, 1H), 8.43 (s, 1H), 8.10 (s, 1H), 7.96-7.91 (m, 2H), 7.37 (dd, J = 5.4, 8.7 Hz, 2H), 7.23-7.16 (m, 2H), 5.36 (s, 2H), 4.24-4.18 (m, 1H), 2.73 (dd, J = 5.2, 12.3 Hz, 1H), 2.62 (br d, J = 3.9 Hz, 1H), 2.37-2.31 (m, 1H), 2.21-2.12 (m, 1H). ¹⁹F NMR (376 MHz, d₆-DMSO) δ −114.78. MS: (ESI) m/z [M + H]⁺ 405.3 |

### Example 294:3-[6-[1-[(4-methoxyphenyl)methyl]pyrazol-4-yl]-2-methyl-benzofuran-3-yl]piperidine-2,6-dione

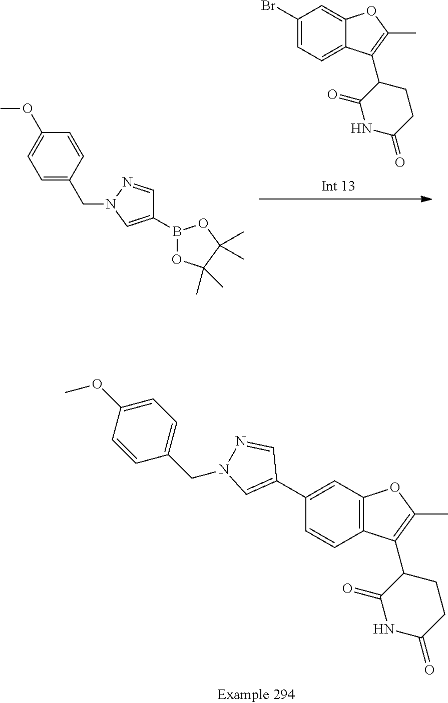

A mixture of 1-[(4-methoxyphenyl)methyl]-4-(4,4,5,5-tetramethyl-1,3,2-dioxaborolan-2-yl)pyrazole (70 mg, 222.80 μmol, 1 eq), Int 13 (71.78 mg, 222.80 μmol, 1 eq), K₃PO₄ (70.94 mg, 334.20 μmol, 1.5 eq), XPhos Pd G3 (18.86 mg, 22.28 μmol, 0.1 eq) in dioxane (2 mL) and H₂O (0.5 mL) was degassed and purged with N₂ for 3 times, and then the mixture was stirred at 80° C. for 1.5 h under N₂ atmosphere. The reaction mixture was concentrated. The residue was purified by prep-HPLC to give Example 294 (2.6 mg, 5.86 μmol, 2.63% yield) as a light yellow solid. ¹H NMR (400 MHZ, do-DMSO) δ 10.91 (s, 1H), 8.24 (s, 1H), 7.91 (s, 1H), 7.67 (s, 1H), 7.38-7.24 (m, 4H), 6.91 (d, J=8.4 Hz, 2H), 5.24 (s, 2H), 4.12-4.08 (m, 1H), 3.72 (s, 2H), 2.79-2.78 (m, 3H), 2.35-2.32 (m, 1H), 1.97-1.94 (m, 1H). MS: (ESI) m/z calculated for C₂₅H₂₃N₃O₄ [M+H]⁺430.1, found 430.0.

Examples 295 was prepared following the similar procedure that was used for preparing Example 294, starting from Int-13 and its corresponding boronic acid or ester intermediate, with proper modifications. Characterizations of Examples 295 are provided in Table 12.

TABLE 12

| Example | | |
|---|---|---|
| ID | Structure | NMR/MS |
| 295 | 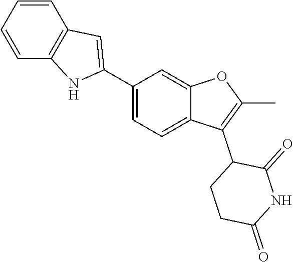 [SMILES: Cc1oc2cc(-c3cc4ccccc4[nH]3)ccc2c1C1CCC(=O)NC1=O] | ¹H NMR (400 MHz, d₆-DMSO) δ 11.51 (s, 1H), 10.93 (s, 1H), 7.96 (s, 1H), 7.70 (d, J = 8.0 Hz, 1H), 7.68-7.38 (m, 3H), 7.08-7.00 (m, 1H), 6.89-6.92 (m, 1H), 6.91 (s, 1H), 4.17-4.12 (m, 1H), 2.80-2.75 (m, 1H), 2.60-2.59 (m, 1H), 2.43 (s, 3H), 2.39-2.38 (m, 1H), 2.00-1.99 (m, 1H). MS: (ESI) m/z [M + H]⁺ 359.0 |

### Section III: Biology Activities of the Compounds of the Application

### 1. Homogeneous Time-Resolved Fluorescence (HTRF) Assay

Cereblon binding of the compounds of the application was measured by HTRF assay (Cisbio). The HTRF assay was performed in OptiPlate-384 white plates with a total volume of 20 mL each well. 5 μL compounds (final con. of 1.6 uM) were added to the plate with 5 μL Human WT GST-tagged Cereblon. Then 10 μL of HTRF detection reagents including 5 μL Anti-GST-Eu cryptate beads (donor) and 5 μL Thalidomide-Red labelled with XL665 (acceptor) were added to the mixture and incubated at room temperature for 3 hours. Compounds that compete with the Thalidomide in binding Cereblon can prevent energy transfer from donor to acceptor. The signals from the plates were detected by microplate reader (PerkinElmer, EnVision, USA) for optimal signal detection at 620 nm (donor) and 665 nm (acceptor). The ratio of 665 nm/620 nm was calculated for each compound to indicate its Cereblon binding ability (the smaller the ratio suggests the better binding ability). The results were shown in Table 13.

TABLE 13
Cereblon binding measured in the HTRF assay

| Example ID | Ratio | Grade |
|---|---|---|
| 2 | 0.15 | +++ |
| 3 | 0.02 | +++ |
| 4 | 0.05 | +++ |
| 6 | 0.10 | +++ |
| 7 | 0.11 | +++ |
| 11 | 0.09 | +++ |
| 72 | 0.07 | +++ |
| 73 | 0.22 | +++ |
| 74 | 0.12 | +++ |
| 97 | 0.27 | +++ |
| 44 | 0.07 | +++ |
| 48 | 0.12 | +++ |
| 70 | 0.13 | +++ |
| 88 | 0.11 | +++ |
| 89 | 0.14 | +++ |
| 103 | 0.23 | +++ |

+++ represents an HTRF ratio value of <=0.4; ++ represents an HTRF ratio value of >0.4 and <=0.8; and + represents an HTRF ratio value of >0.8.

2a. Target Protein Degradation Assay

Activity for targeted protein (e.g., HuR) degradation was examined by Western

blot assays. KP4 cells were first treated with compounds for 6 hours at 0.1 μM, then collected and lysed by RIPA buffer. Primary antibodies were prepared in 5% BSA (1:1000) and incubated at 4° C. overnight. Secondary antibodies were prepared with 5% skimmed milk (dilution range 1:2000-1:3000) and incubated at room temperature for 1 h. The PVDF membrane was washed three times with TBST buffer and detected by chemiluminescence. The results were shown in Table 14.

TABLE 14
Protein degradation measured by Western blot assay

| Example | Ranking of HuR | Example | Ranking of HuR |
|---|---|---|---|
| ID | degradation | ID | degradation |
| 1 | D | 2 | B |
| 3 | A | 4 | A |
| 5 | A | 6 | A |
| 7 | A | 8A | A |
| 8B | A | 9 | A |
| 10 | A | 11 | A |
| 12 | A | 13 | B |
| 14 | A | 15 | A |
| 16 | D | 17 | D |
| 18 | D | 19 | D |
| 20 | C | 21 | C |
| 22 | C | 23 | A |
| 24 | B | 25 | C |
| 26 | B | 27 | C |
| 28 | C | 29 | B |
| 30 | C | 31 | B |
| 32 | C | 33 | C |
| 34 | B | 35 | B |
| 36 | B | 37 | B |
| 38 | B | 39 | A |
| 40 | A | 41 | B |
| 42 | A | 43 | A |
| 44 | A | 45 | A |
| 46 | B | 47 | A |
| 48 | A | 49 | A |
| 50 | A | 51 | A |
| 52 | A | 53 | A |
| 54 | B | 55 | B |
| 56 | B | 57 | B |
| 58 | A | 59 | B |
| 60 | B | 61 | A |
| 62 | B | 63 | B |
| 64 | B | 65 | A |
| 66 | A | 67 | A |
| 68 | A | 69 | A |
| 70 | A | 71 | B |
| 72 | B | 73 | A |
| 74 | B | 75 | A |
| 76 | A | 77 | B |
| 78 | D | 79 | B |
| 80 | A | 81 | A |
| 82 | A | 83 | B |
| 84 | B | 85 | B |
| 86 | B | 87 | B |
| 88 | A | 89 | A |
| 90 | A | 91 | A |
| 92 | A | 93 | A |
| 94 | A | 95 | B |
| 96 | A | 97 | A |
| 98 | A | 99 | B |
| 100 | D | 101 | B |
| 102 | C | 103 | B |
| 104 | C | 105 | B |
| 106 | B | 107 | C |
| 108 | B | 109 | B |
| 110 | B | 111 | A |
| 112 | B | 113 | B |
| 114 | B | 115 | B |
| 116 | B | 117 | B |
| 118 | B | 119 | B |
| 120 | B | 121 | A |
| 122 | A | 123 | A |
| 124 | A | 125 | A |
| 126 | D | 127 | B |
| 128 | A | 129 | B |
| 130 | A | 131 | A |
| 132 | B | 133 | A |
| 134 | A | 135 | A |
| 136 | A | 137 | A |
| 138 | A | 139 | A |
| 140 | A | 141 | B |
| 142 | A | 143 | A |
| 144 | A | 145 | A |
| 146 | B | 147 | A |
| 148 | A | 149 | B |
| 150 | A | 151 | A |
| 152 | B | 153 | A |
| 154 | A | 155 | A |
| 156 | A | 157 | A |
| 158 | B | 159 | A |
| 160 | B | 161 | A |
| 162 | A | 163 | B |
| 164 | A | 165 | A |
| 166 | A | 167 | A |
| 168 | A | 169 | A |
| 170 | B | 171 | A |
| 172 | A | 173 | A |
| 174 | B | 175 | B |
| 176 | A | 177 | B |
| 178 | A | 179 | A |
| 180 | A | 181 | A |
| 182 | A | 183 | A |
| 184 | A | 185 | B |
| 186 | A | 187 | A |
| 188 | A | 189 | A |
| 190 | A | 191 | A |
| 192 | A | 193 | A |
| 194 | A | 195 | A |
| 196 | A | 197 | A |
| 198 | A | 199 | A |
| 200 | A | 201 | A |
| 202 | A | 203 | A |
| 204 | A | 205 | A |
| 206 | A | 207 | A |
| 208 | A | 209 | A |
| 210 | A | 211 | A |
| 212 | A | 213 | A |
| 214 | A | 215 | B |
| 216 | B | 217 | B |
| 218 | A | 219 | A |
| 220 | A | 221 | A |
| 222 | B | 223 | B |
| 224 | A | 225 | A |
| 226 | B | 227 | A |
| 228 | A | 229 | A |
| 230 | A | 231 | A |
| 232 | A | 233 | A |
| 234 | A | 235 | A |
| 236 | A | 237 | A |
| 238 | A | 239 | A |
| 240 | B | 241 | A |
| 242 | A | 243 | A |
| 244 | A | 245 | B |
| 246 | A | 247 | A |
| 248 | A | 249 | B |
| 250 | B | 251 | A |
| 252 | A | 253 | A |
| 254 | A | 255 | A |
| 256 | A | 257 | B |
| 258 | B | 259 | A |
| 260 | A | 261 | A |
| 262 | A | 263 | A |
| 264 | A | 265 | A |
| 266 | A | 267 | A |
| 268 | A | 269 | A |
| 270 | A | 271 | A |
| 272 | A | 273 | A |
| 274 | A | 275 | A |
| 278 | C | 279 | D |
| 280 | D | 281 | D |
| 282 | D | 283 | D |
| 284 | D | 285 | D |
| 286 | D | 287 | D |
| 288 | C | 289 | B |
| 290 | C | 291 | D |
| 292 | D | 293 | D |
| 294 | D | 295 | D |
| 298 | D | 299 | D |
| 300 | D | 313 | D |
| 322 | D | 323 | C |
| 341 | D | 342 | C |
| 343 | B | 344 | D |
| 345 | C | 346 | B |
| 347 | B | 348 | B |
| 349 | C | 350 | B |
| 351 | D | 352 | D |
| 353 | B | 354 | B |
| 355 | D | 356 | D |
| 357 | B | 358 | C |
| 359 | D | 360 | B |
| 361 | B | 362 | B |
| 363 | C | 364 | D |
| 365 | D | 366 | D |
| 367 | C | 368 | B |
| 369 | D | 370 | D |
| 371 | B | 372 | C |
| 373 | B | 374 | C |
| 375 | B | 376 | C |
| 377 | C | 378 | D |
| 379 | B | 380 | C |
| 381 | B | 382 | D |
| 383 | C | 384 | D |
| 385 | D | 386 | B |
| 390 | D | 391 | D |
| 393 | B | 394 | D |
| 395 | B | 396 | B |
| 397 | D | 398 | D |
| 399 | D | 400 | D |
| 401 | D | 403 | D |
| 405 | D | 406 | C |
| 407 | D | 408 | D |
| 409 | C | 410 | D |
| 411 | C | 412 | C |
| 413 | B | 414 | B |
| 415 | B | 416 | B |
| 417 | C | 418 | B |
| 419 | D | 420 | C |
| 421 | C | 422 | D |
| 423 | B | 424 | D |
| 425 | D | 426 | B |
| 427 | D | 429 | B |
| 430 | D | 436 | D |
| 437 | D | 438 | C |
| 439 | B | 440 | C |
| 450 | C | 451 | B |
| 452 | D | 455 | C |
| 456 | D | 457 | D |
| 460 | C | 462 | D |
| 464 | C | 465 | D |
| 466 | C | 467 | B |
| 468 | D | 471 | B |
| 472 | D | 473 | B |
| 474 | C | 475 | D |
| 476 | D | 477 | B |
| 478 | C | 479 | C |
| 480 | B | 482 | B |
| 483 | D | 484 | B |
| 485 | C | 486 | C |
| 487 | B | 488 | B |
| 489 | D | 490 | C |
| 491 | C | 492 | D |
| 493 | D | 494 | B |
| 495 | B | 496 | D |
| 497 | D | | |

A represents a remaining percentage of the protein compared to the blank control of <=30%; B represents a remaining percentage of the protein >30% and <=70%; C represents a remaining percentage of the protein >70% and <=90%; and D represents a remaining percentage of the protein >90%.

2b. Target Protein Degradation Assay

Activity for targeted protein (e.g., HuR) degradation was examined by Western blot assays. JHH7 cells were first treated with compounds for 6 hours at 1 μM, then collected and lysed by RIPA buffer. Primary antibodies were prepared in 5% BSA (1:1000) and incubated at 4° C. overnight. Secondary antibodies were prepared with 5% skimmed milk (dilution range 1:2000-1:3000) and incubated at room temperature for 1 h. The PVDF membrane was washed three times with TBST buffer and detected by chemiluminescence. The results were shown in Table 15.

TABLE 15
Protein degradation measured by Western blot assay

| Example | Ranking of HuR | Example | Ranking of HuR |
|---|---|---|---|
| ID | degradation | ID | degradation |
| 276 | C | 277 | C |
| 296 | D | 297 | D |
| 301 | C | 302 | C |
| 303 | B | 304 | B |
| 305 | A | 306 | A |
| 307 | D | 308 | A |
| 309 | D | 310 | D |
| 311 | D | 312 | B |
| 314 | B | 315 | C |
| 316 | C | 317 | C |
| 318 | C | 319 | C |
| 320 | D | 321 | D |
| 324 | C | 325 | D |
| 326 | D | 327 | B |
| 328 | C | 329 | C |
| 330 | C | 331 | C |
| 332 | C | 333 | C |
| 334 | C | 335 | C |
| 336 | D | 337 | D |
| 338 | C | 339 | D |
| 340 | C | 387 | B |
| 388 | B | 389 | D |
| 392 | D | 402 | B |
| 404 | D | 431 | C |
| 432 | D | 433 | C |
| 434 | D | 435 | C |
| 441 | D | 442 | D |
| 443 | B | 444 | B |
| 445 | B | 446 | B |
| 447 | D | 448 | D |
| 449 | B | 453 | C |
| 454 | B | 458 | D |
| 459 | D | 461 | D |
| 463 | A | 469 | D |
| 470 | C | 481 | B |

A represents a remaining percentage of the protein compared to the blank control of <=30%; B represents a remaining percentage of the protein >30% and <=70%; C represents a remaining percentage of the protein >70% and <=90%; and D represents a remaining percentage of the protein >90%.

### 3. Anti-Proliferation Activity

KP4 (pancreatic cancer cell line, wild type and CRBN—KO), JHH7 (liver cancer cell line, wild type and CRBN—KO) and OVTOKO (ovarian cancer cell line, wild type and CRBN—KO) were treated with compounds of the application for 7-14 days at gradient dosages (0.1, 0.3, 1, 3, 10 μM). The relative viability of compound-treated cancer cells was determined by CellTiter-Glo Luminescent Cell Viability Assay (CTG) and analyzed by Graphpad Prism.

Prepare plates: Cells were plated at the optimal density in a 24-well plate in triplicates for each drug concentration, in a media volume of 1 mL; the optimal seeding density was pre-determined, to achieve around 80%-90% cell confluence with compound treatment in 7 days (Day-1).

Treatment: The next day, serial dilutions of the compound stocks in DMSO were made and diluted in complete media at 1:1000; for each dosage, 3.3 mL of compound-containing media was made and then media in each well was replaced with 1 ml of compound-containing media accordingly (Day 0). After 3 or 4 days, media was replenished with fresh media containing relevant compound concentration to each well (Day 3 or 4).

Passage and determine the relative viability of the cells: At day 7, cells were split at an appropriate ratio, according to the control well from each cell line. The cell layer was briefly rinsed with 0.5 ml of PBS. 70 μl of 0.05% Trypsin solution was added to individual well and cells were incubated at 37° C. for detachment. 430 μl of fresh media was added and cells were aspirated by gently pipetting. An appropriate volume of cells was transferred to a new 24-well plate and fresh media was added to make a total volume of 1 mL. Meanwhile, some cells were aliquoted to opaque-wall 384-well plate for CTG assay (25 μL cells plus 25 μL reagent for 10 min, then the chemiluminescence was determined).

A graph according to 1- or 2-round of treatment (7-day or 14-day) was plotted in Graph Prism. The results were shown in Table 16.

TABLE 16
Anti-proliferation activity (inhibition effect)
of the compounds in various cell lines

| Example ID | Ranking of activities |
|---|---|
| 2 | + |
| 3 | +++ |
| 4 | +++ |
| 6 | +++ |
| 7 | +++ |
| 11 | +++ |
| 72 | ++ |
| 73 | +++ |
| 74 | +++ |
| 97 | +++ |
| 44 | +++ |
| 48 | +++ |
| 70 | +++ |
| 88 | +++ |
| 89 | + |
| 103 | + |

+++ represents an IC30 value of <=200 nM in Colo205 cells and inhibition activity in at least one cell line, ++ represents an IC30 value of >200 nM and <=1000 nM and inhibition activity in at least one cell line, + represents an IC30 value of >1000 nM and inhibition activity in at least one cell line.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A compound of Formula I: 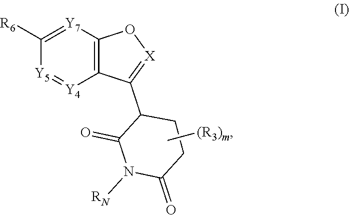
or a pharmaceutically acceptable salt, solvate, prodrug, stereoisomer, or tautomer thereof, wherein:
X is N or CR_{X};
Y₄ is N or CR₄;
Y₅ is N or CR₅;
Y₇ is N or CR₇, provided that no more than one of Y₄, Y₅, and Y₇ is N;
R_{X}, R₄, R₅, and Ry each independently is H, C₁-C₆ alkyl, C₁-C₆ haloalkyl, or halogen;
m is 0, 1, 2, 3, or 4;
each R₃ is independently C₁-C₆ alkyl, C₁-C₆ haloalkyl, C₁-C₆ alkoxy, C₁-C₆ haloalkoxy, OH, or halogen;
R_{N} is H or C₁-C₆ alkyl;
R₆ is R₆′ or (CH₂)₁₋₃—R₆′;
R₆′ is heterocyclyl comprising a 4- to 6-membered ring comprising a nitrogen atom and optionally additional 1 to 3 heteroatoms selected from N, O, and S, C₆-C₁₀ aryl, heteroaryl comprising a 5- or 6-membered ring comprising a nitrogen atom and optionally additional 1 to 3 heteroatoms selected from N, O, and S, or fused-ring heteroaryl comprising a 5- or 6-membered ring comprising a nitrogen atom and optionally additional 1 to 3 heteroatoms selected from N, O, and S and a 5- or 6-membered ring optionally comprising 1 to 4 heteroatoms selected from N, O, and S, wherein the heterocyclyl, aryl, heteroaryl, or fused-ring heteroaryl is optionally substituted with one or more R₆₁; and
each R₆₁ is independently
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN, ═O, C(O)R₆₂, C(O)OR₆₂, C(O)NR₆₃R₆₄,
L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁-2NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms,
wherein:
L is absent, or a linker selected from O, (CR_{L1}R_{L2})ₙ, O—(CR_{L1}R_{L2})ₙ, (CR_{L1}R_{L2})ₙ—O, and S(O)₂;
each R_{L1} and each R_{L2} is independently H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
each R_{A} is independently halogen, OH, C₁-C₆ alkyl, C₁-C₆ alkoxy, C₁-C₆ alkyl amino, or di-C₁-C₆ alkyl amino; and
n is 1, 2, or 3; or
two R₆₁, together with the carbon atom(s) to which they are attached, may form C₄-C₁₀ carbocyclyl, heterocyclyl comprising one 4- to 6-membered and 1 to 3 heteroatoms selected from N, O, and S, spiro- or fused-heterocyclyl comprising two 4- to 6-membered rings comprising 1 to 4 heteroatoms selected from N, O, and S, or C₆ aryl, wherein the carbocyclyl, heterocyclyl, spiro- or fused-heterocyclyl, or aryl is optionally substituted with one or more groups independently selected from C₁-C₆ alkyl, C₁-C₆ haloalkyl, C₁-C₆ alkoxy, C₁-C₆ haloalkoxy, C₁-C₆ alkyl amino, di-C₁-C₆ alkyl amino, OH, amino, halogen, CN, and ═O; and
each R₆₂, each R₆₃, and each R₆₄ is independently H, C₁-C₆ alkyl, or C₁-C₆ haloalkyl.

2. The compound of claim 1, of any one of Formulae II-IX: 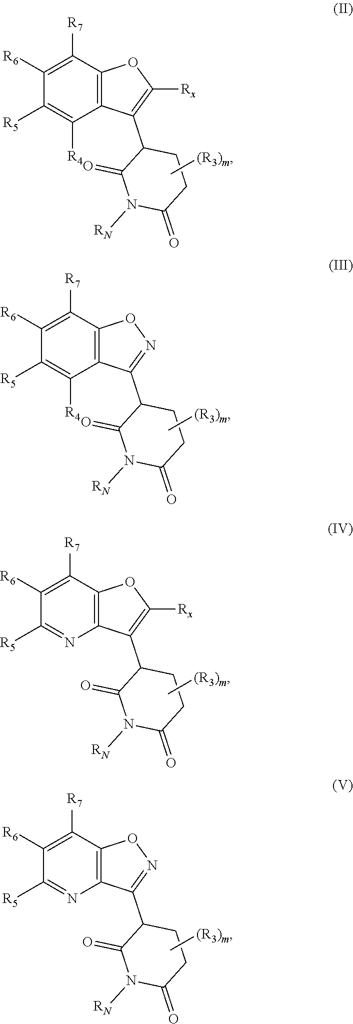 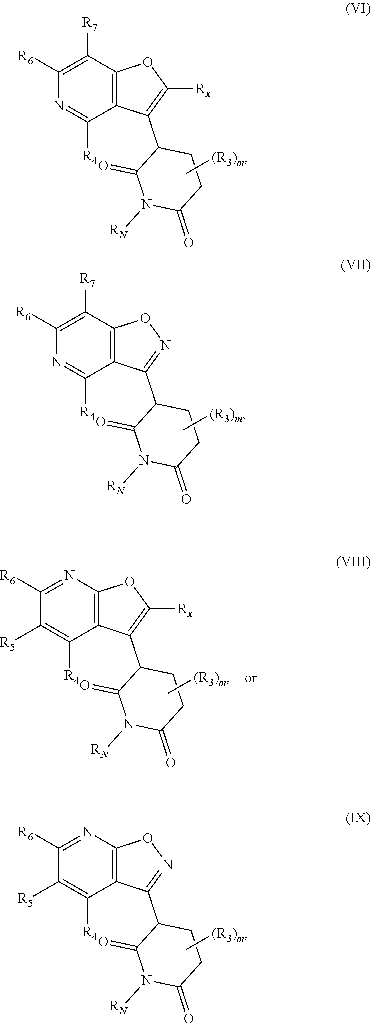
or a pharmaceutically acceptable salt, solvate, prodrug, stereoisomer, or tautomer thereof.

3. The compound of claim 1 or 2, of Formula II or III: 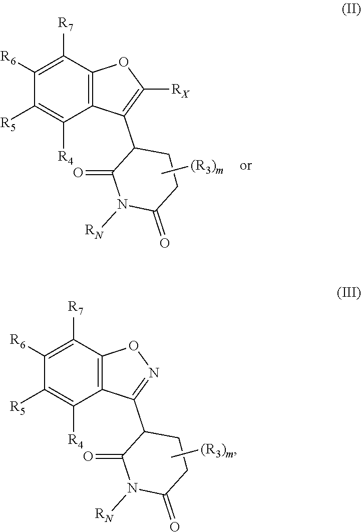
or a pharmaceutically acceptable salt, solvate, prodrug, stereoisomer, or tautomer thereof.

4. The compound of any one of claims 1-3, wherein R_{X} is H.

5. The compound of any one of claims 1-4, wherein m is 0.

6. The compound of any one of claims 1-5, wherein R_{N} is H.

7. The compound of any one of claims 1-6, wherein R& is (CH₂)₁₋₃—R₆′.

8. The compound of any one of claims 1-6, wherein R₆ is R₆′.

9. The compound of any one of claims 1-8, wherein R₆′ is heteroaryl comprising a 5- or 6-membered ring comprising a nitrogen atom and optionally additional 1 to 3 heteroatoms selected from N, O, and S, or fused-ring heteroaryl comprising a 5- or 6-membered ring comprising a nitrogen atom and optionally additional 1 to 3 heteroatoms selected from N, O, and S and a 5- or 6-membered ring optionally comprising 1 to 4 heteroatoms selected from N, O, and S, wherein the heteroaryl or fused-ring heteroaryl is optionally substituted with one or more R₆₁.

10. The compound of any one of claims 1-9, wherein R₆′ is heteroaryl comprising a 5-membered ring comprising a nitrogen atom and optionally additional 1 to 3 heteroatoms selected from N, O, and S, wherein the heteroaryl is optionally substituted with one or more R₆₁.

11. The compound of any one of claims 1-9, wherein R₆′ is fused-ring heteroaryl comprising a 5- or 6-membered ring comprising a nitrogen atom and optionally additional 1 to 3 heteroatoms selected from N, O, and S and a 5- or 6-membered ring optionally comprising 1 to 4 heteroatoms selected from N, O, and S, wherein the fused-ring heteroaryl is optionally substituted with one or more R₆₁.

12. The compound of any one of claims 1-9 and 11, wherein R₆′ is fused-ring heteroaryl comprising a 5-membered ring comprising a nitrogen atom and optionally additional 1 to 3 heteroatoms selected from N, O, and S and a 5- or 6-membered ring optionally comprising 1 to 4 heteroatoms selected from N, O, and S, wherein the fused-ring heteroaryl is optionally substituted with one or more R₆₁.

13. The compound of any one of claims 1-9, 11, and 12, wherein R₆′ is fused-ring heteroaryl comprising a 5-membered ring comprising a nitrogen atom and optionally additional 1 to 3 heteroatoms selected from N, O, and S and a 6-membered ring optionally comprising 1 to 4 heteroatoms selected from N, O, and S, wherein the fused-ring heteroaryl is optionally substituted with one or more R₆₁.

14. The compound of any one of claims 1-9, wherein R₆′ is C₆-C₁₀ aryl optionally substituted with one or more R₆₁.

15. The compound of any one of claims 1-9, wherein R₆′ is heterocyclyl comprising a 5- or 6-membered ring comprising a nitrogen atom and optionally additional 1 to 3 heteroatoms selected from N, O, and S, wherein the heterocyclyl is optionally substituted with one or more R₆₁.

16. The compound of any one of claims 1-9 and 15, wherein R₆′ is heterocyclyl comprising a 5- or 6-membered ring comprising a nitrogen atom and optionally additional 1 to 3 heteroatoms selected from N, O, and S, wherein the heterocyclyl is optionally substituted with one or more R₆₁, and wherein the heterocyclyl is fully saturated or partially saturated.

17. The compound of any one of claims 1-16, wherein at least one R₆₁ is
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN, ═O,
C(O)R₆₂, C(O)OR₆₂, C(O)NR₆₃R₆₄,
L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted.

18. The compound of any one of claims 1-17, wherein at least one R₆₁ is
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN, ═O, C(O)R₆₂, C(O)OR₆₂, or C(O)NR₆₃R₆₄.

19. The compound of any one of claims 1-17, wherein at least one R₆₁ is L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted.

20. The compound of any one of claims 1-17, wherein at least one R₆₁ is L-C₆-C₁₀ aryl or L-heteroaryl comprising one or two 5- or 6-membered rings and 1 to 4 heteroatoms selected from N, O, and S, wherein the aryl or heteroaryl is optionally substituted.

21. The compound of any one of claims 1-17, wherein R₆′ is substituted with two or more R₆₁, and at least two R₆₁, together with the carbon atom(s) to which they are attached, may form C₄-C₁₀ carbocyclyl, heterocyclyl comprising one 4- to 6-membered and 1 to 3 heteroatoms selected from N, O, and S, spiro- or fused-heterocyclyl comprising two 4- to 6-membered rings comprising 1 to 4 heteroatoms selected from N, O, and S, or C₆ aryl, wherein the carbocyclyl, heterocyclyl, spiro- or fused-heterocyclyl, or aryl is optionally substituted with one or more groups independently selected from C₁-C₆ alkyl, C₁-C₆ haloalkyl, C₁-C₆ alkoxy, C₁-C₆ haloalkoxy, C₁-C₆ alkyl amino, di-C₁-C₆ alkyl amino, OH, amino, halogen, CN, and ═O.

22. The compound of any one of claims 1-21, wherein R₅ is H.

23. The compound of any one of claims 1-21, wherein R₅ is halogen.

24. The compound of claim 23, wherein R₅ is F.

25. The compound of any one of claims 1-21, wherein R₅ is C₁-C₆ alkyl or C₁-C₆ haloalkyl.

26. The compound of any one of claims 1-25, wherein R₄ is H.

27. The compound of any one of claims 1-25, wherein R₄ is halogen.

28. The compound of claim 27, wherein R₄ is F.

29. The compound of any one of claims 1-25, wherein R₄ is C₁-C₆ alkyl or C₁-C₆ haloalkyl.

30. The compound of any one of claims 1-29, wherein R₇ is H.

31. The compound of any one of claims 1-29, wherein R₇ is halogen.

32. The compound of claim 31, wherein R₇ is F.

33. The compound of any one of claims 1-29, wherein R₇ is C₁-C₆ alkyl or C₁-C₆ haloalkyl.

34. The compound of claim 1, of Formula Ia: 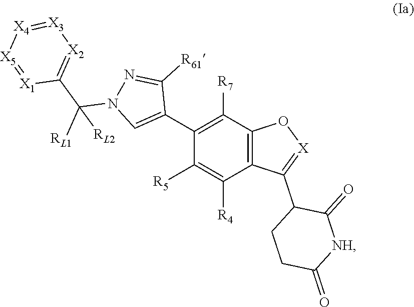
or a pharmaceutically acceptable salt, solvate, prodrug, stereoisomer, or tautomer thereof, wherein:
X is N or CR_{X};
R_{X}, R₄, R₅, and R₇ each independently is H, C₁-C₆ alkyl, C₁-C₆ haloalkyl, or halogen;
R₆₁′ is
H,
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN,
C(O)R₆₂, C(O)OR₆₂, or C(O)NR₆₃R₆₄;
R₆₂, R₆₃, and R₆₄ each independently is H, C₁-C₆ alkyl, or C₁-C₆ haloalkyl;
R_{L1} and R_{L2} each independently is H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
X₁ is N or CR_{X1;}
X₂ is N or CR_{X2;}
X₃ is N or CR_{X3};
X₄ is N or CR_{X4};
X₅ is N or CR_{X5}, provided that no more than four of X₁, X₂, X₃, X₄, and X₅ are N;
R_{X1}, R_{X2}, R_{X3}, R_{X4}, and R_{X5} each independently is H, C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein any two of R_{X1}, R_{X2}, R_{X3}, R_{X4}, and R_{X5} attached to adjacent carbon atoms, together with the carbon atoms to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in any of R_{X1}, R_{X2}, R_{X3}, R_{X4}, and R_{X5} may be replaced with one or more deuterium atoms; and
each R_{A} is independently halogen, OH, C₁-C₆ alkyl, C₁-C₆ alkoxy, C₁-C₆ alkyl amino, or di-C₁-C₆ alkyl amino.

35. The compound of claim 34, wherein R₆₁′ is H or halogen.

36. The compound of claim 34 or 35, wherein R_{L1} and R_{L2} each independently is H, CH₃, CH₂CH₃, or CH(CH₃)₂.

37. The compound of any one of claims 34-36, wherein X₁, X₂, and X₃ are each CH.

38. The compound of any one of claims 34-36, wherein X₁ and X₂ are each N, and X₃ is CH.

39. The compound of any one of claims 34-36, wherein X₂ and X₃ are each N, and X₁ is CH.

40. The compound of any one of claims 34-39, wherein R₄ and R₇ each is H, and R₅ is H or F.

41. The compound of any one of claims 34-39, wherein R₄ is F, and R₅ and R₇ are each independently H or F.

42. The compound of claim 1, of Formula Ib: 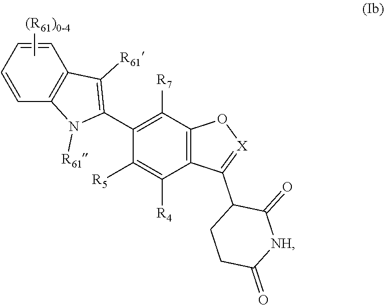
or a pharmaceutically acceptable salt, solvate, prodrug, stereoisomer, or tautomer thereof, wherein:
X is N or CR_{X};
R_{X}, R₄, R₅, and R₇ each independently is H, C₁-C₆ alkyl, C₁-C₆ haloalkyl, or halogen;
each R₆₁″ is independently
H,
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN,
C(O)R₆₂, C(O)OR₆₂, C(O)NR₆₃R₆₄,
L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N})S(O)₂—C₁-C₆ alkyl, N(R_{N})S(O)₂—C₃-C₆ cycloalkyl, S(O)₁₋₂NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR (2)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms
each R₆₁ is independently
C₁-C₆ alkyl optionally substituted with one or more groups independently selected from C₁-C₆ alkyl amino, di-C₁-C₆ alkyl amino, halogen, OH, amino, C₆-C₁₀ aryl, and heteroaryl comprising one or two 5- or 6-membered rings and 1 to 4 heteroatoms selected from N, O, and S,
C₁-C₆ alkoxy, C₁-C₆ haloalkoxy, C₁-C₆ alkyl amino, di-C₁-C₆ alkyl amino, OH, amino, NO₂, halogen, CN, ═O,
C(O)R₆₂, C(O)OR₆₂, C(O)NR₆₃R₆₄,
L-C₆-C₁₀ aryl, L-heteroaryl comprising one or two 5- or 6-membered rings and 1 to 4 heteroatoms selected from N, O, and S, L-C₃-C₆ carbocyclyl, or L-heterocyclyl comprising one or two 4- to 6-membered rings and 1 to 4 heteroatoms selected from N, O, and S, wherein the aryl, heteroaryl, carbocyclyl, or heterocyclyl is optionally substituted with one or more groups independently selected from:
C₁-C₆ alkyl, C₁-C₆ haloalkyl, (CH₂)₀₋₃—C₁-C₆ alkoxy optionally substituted with one or more substituents selected from halogen, C₁-C₆ alkyl amino, di-C₁-C₆ alkyl amino, and C₁-C₆ alkoxy, O—(CH₂)₀₋₃—C₃-C₆ carbocyclyl, O—(CH₂)₀₋₃-heterocyclyl comprising one or two 3- to 6-membered rings and 1 to 4 heteroatoms selected from N, O, and S, O—(CH₂)₀₋₃—C₆-C₁₀ aryl, O—(CH₂)₀₋₃-heteroaryl comprising one or two 5- or 6-membered rings and 1 to 4 heteroatoms selected from N, O, and S, C₁-C₆ alkyl amino wherein alkyl is optionally substituted with one or more substituents selected from C₁-C₆ alkyl amino, di-C₁-C₆ alkyl amino, and C₁-C₆ alkoxy, di-C₁-C₆ alkyl amino wherein each alkyl is independently optionally substituted with one or more substituents selected from C₁-C₆ alkyl amino, di-C₁-C₆ alkyl amino, and C₁-C₆ alkoxy, OH, amino, NO₂, halogen, CN, ═O, N(R_{N})C(O)R₆₂, N(R_{N})C(O)OR₆₂, N(R_{N}) S(O)₂—C₁-C₆ alkyl, N(R_{N}) S(O)₂—C₃-C₆ cycloalkyl, S(O)₁-2NH(C₁-C₆ alkyl), S(O)₁₋₂N(C₁-C₆ alkyl)₂, S(O)₀₋₂—C₁-C₆ alkyl, C(O)R₆₂, C(O)OR₆₂, C(O)NR₆₃R₆₄, P(═O) (OR₆₂)₂, C₃-C₆ carbocyclyl, heterocyclyl comprising one or two 3- to 6-membered rings and 1 to 4 heteroatoms selected from N, O, and S, C₆-C₁₀ aryl, and heteroaryl comprising one or two 5- or 6-membered rings and 1 to 4 heteroatoms selected from N, O, and S, wherein two of the substituent groups, together with the carbon atom(s) to which they are attached, may form C₅-C₆ carbocyclyl or heterocyclyl comprising a 5- or 6-membered ring and 1 or 2 heteroatoms selected from N, O, and S and wherein the carbocyclyl or heterocyclyl is optionally substituted with one or more R_{A}, and wherein one or more hydrogen atoms in the substituent group may be replaced with one or more deuterium atoms,
wherein:
L is absent, or a linker selected from O, (CR_{L1}R_{L2})ₙ, O—(CR_{L1}R_{L2})ₙ, (CR_{L1}R_{L2})ₙ—O, and S(O)₂;
each R_{L1} and each R_{L2} is independently H, CH₃, CH₂CH₃, or CH(CH₃)₂, or R_{L1} and R_{L2}, together with the carbon atom to which they are attached, may form C(O), C₃-C₆ cycloalkyl, or heterocyclyl comprising a 4- to 6-membered ring comprising and 1 to 3 heteroatoms selected from N, O, and S, wherein one or more hydrogen atoms in any of R_{L1} and R_{L2} may be replaced with one or more deuterium atoms;
each R_{A} is independently halogen, OH, C₁-C₆ alkyl, C₁-C₆ alkoxy, C₁-C₆ alkyl amino, or di-C₁-C₆ alkyl amino; and
n is 1, 2, or 3; or
two R₆₁, together with the carbon atom(s) to which they are attached, may form C₄-C₁₀ carbocyclyl, heterocyclyl comprising one 4- to 6-membered and 1 to 3 heteroatoms selected from N, O, and S, spiro- or fused-heterocyclyl comprising two 4- to 6-membered rings comprising 1 to 4 heteroatoms selected from N, O, and S, or C₆ aryl, wherein the carbocyclyl, heterocyclyl, spiro- or fused-heterocyclyl, or aryl is optionally substituted with one or more groups independently selected from C₁-C₆ alkyl, C₁-C₆ haloalkyl, C₁-C₆ alkoxy, C₁-C₆ haloalkoxy, C₁-C₆ alkyl amino, di-C₁-C₆ alkyl amino, OH, amino, halogen, CN, and —O; and
each R₆₂, each R₆₃, and each R₆₄ is independently H, C₁-C₆ alkyl, or C₁-C₆ haloalkyl.

43. The compound of claim 42, wherein each R₆₁″ is H.

44. The compound of claim 42 or 43, wherein R₄ and Ry each is H, and R, is H or F.

45. The compound of any one of claims 42-44, wherein each R₆₁ is independently C₁-C₆ alkyl, C₁-C₆ haloalkyl, C₁-C₆ alkoxy, C₁-C₆ haloalkoxy, OH, or halogen.

46. A compound selected from Table A, or a pharmaceutically acceptable salt, solvate, prodrug, stereoisomer, or tautomer thereof.

47. The compound of claim 46, selected from Table B, or a pharmaceutically acceptable salt, solvate, prodrug, stereoisomer, or tautomer thereof.

48. The compound of claim 46, selected from Table C, or pharmaceutically acceptable salt, solvate, prodrug, stereoisomer, or tautomer thereof.

49. A pharmaceutical composition comprising a compound of any one of claims 1-48, or a pharmaceutically acceptable salt or solvate thereof, and a pharmaceutically acceptable carrier or excipient.

50. A method of treating or preventing a diseases or disorder in a subject in need thereof, comprising administering to the subject a therapeutically effective amount of a compound of any one of claims 1-48, or a pharmaceutically acceptable salt or solvate thereof.
